(19) United States
(12) Patent Application Publication
(10) Pub. No.: US 2009/0304590 A1
(43) Pub. Date: Dec. 10, 2009

(54) Therapeutic compositions and methods

(75) Inventors: Davinder Singh Gill, Andover, MA (US); Laird Bloom, Needham, MA (US); Maximillian T. Follettie, Belmont, MA (US); Fionnuala McAleese, Cambridge, MA (US); Sreekumar Kodangattil, Westbrook, CT (US); John Francis DiJoseph, Woodbridge, NJ (US); Nitin K. Damle, Upper Saddle River, NJ (US); Peter R. Baum, Seattle, WA (US); Peter A. Thompson, Bellevue, WA (US); John C. Kumer, Seattle, WA (US); Alan F. Wahl, Mercer Island, WA (US); Paul A. Algate, Issaquah, WA (US); Sateesh Kumar Natarajan, Redmond, WA (US)

Correspondence Address: ROPES & GRAY LLP, PATENT DOCKETING 39/361, 1211 AVENUE OF THE AMERICAS, NEW YORK, NY, 10036-8704, US

(73) Assignees: Wyeth, Madison, NJ (US); Trubion Pharmaceuticals, Inc., Seattle, WA (US)

(21) Appl. No.: 12/290,176

(22) Filed: Oct. 27, 2008

Related U.S. Application Data
(63) Continuation-in-part of application No. 12/156,159, filed on May 29, 2008.
Application No. 12/156,159 is a continuation-in-part of application No. PCT/US2008/006905, filed on May 29, 2008.
(60) Provisional application No. 61/000,511, filed on Oct. 25, 2007.
(60) Provisional application No. 61/062,433, filed on Jan. 24, 2008.
(60) Provisional application No. 60/932,302, filed on May 29, 2007.
(60) Provisional application No. 60/932,302, filed on May 29, 2007.

(51) Int. Cl. A61K 38/00; C07K 2/00; C07K 16/18; C07H 21/00; C12N 5/10; C12P 21/00; A61K 31/704; A61K 31/7068; A61K 33/24; A61K 31/337; A61K 49/00

(52) U.S. Cl. 424/9.1; 530/300; 530/389.1; 530/350; 536/23.1; 514/12; 435/358; 435/369; 435/69.1; 514/34; 514/49; 424/649; 514/449

(57) ABSTRACT

The present application provides novel binding proteins, including human binding proteins that specifically bind to the human ErbB2.

### CROSS REFERENCE TO RELATED APPLICATIONS

This application claims the benefit of U.S. Provisional Applications 61/000,511, filed Oct. 25, 2007 and 61/062,433, filed Jan. 24, 2008, and of PCT/US2008/006905 and Unites States application Ser. No. 12/156,159, both filed May 29, 2008.

### FIELD OF THE INVENTION

This invention relates to binding proteins that bind erythroblastic leukemia viral oncogene homolog 2 (ErbB2), in particular, human ErbB2 (also known as HER2), and their use in regulating ErbB2-associated activities. The binding proteins disclosed herein are useful in diagnosing, preventing, and/or treating ErbB2 associated disorders, e.g., hyperproliferative disorders, including cancer, and autoimmune disorders, including arthritis.

### BACKGROUND OF THE INVENTION

The ErbB family of receptor tyrosine kinases are important mediators of cell growth, differentiation and survival. The receptor family includes four distinct members including epidermal growth factor receptor (EGFR or ErbB1), HER2 (ErbB2 or p185^{neu}), HER3 (ErbB3) and HER4 (ErbB4 or tyro2). Structurally, the ErbB receptors possess an extracellular domain (with four subdomains, I-IV), a single hydrophobic transmembrane domain, and (except for HER3) a highly conserved tyrosine kinase domain. Crystal structures of EGFR reveal a receptor that adopts one of two conformations. In the “closed” conformation, EGFR is not bound by ligand and the extracellular subdomains II and IV remain tightly apposed, preventing inter-receptor interactions. Ligand binding prompts the receptor to adopt an “open” conformation, in which the EGFR receptor is poised to make inter-receptor interactions.

The ErbB receptors are generally found in various combinations in cells and heterodimerization is thought to increase the diversity of cellular responses to a variety of ErbB ligands. EGFR is bound by at least six different ligands; epidermal growth factor (EGF), transforming growth factor alpha (TGF-α), amphiregulin, heparin binding epidermal growth factor (HB-EGF), betacellulin and epiregulin. A family of heregulin proteins resulting from alternative splicing of a single gene are ligands for ErbB3 and ErbB4. The heregulin family includes alpha, beta and gamma heregulins, neu differentiation factors (NDFs), glial growth factors (GGFs); acetylcholine receptor inducing activity (ARIA); and sensory and motor neuron derived factor (SMDF).

HER2 was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis. Overexpression of ErbB2 (frequently but not uniformly due to gene amplification) has also been observed in other carcinomas including carcinomas of the stomach, endometrium, salivary gland, lung, kidney, colon, thyroid, pancreas and bladder.

HER2 has been suggested to be a ligand orphan receptor. Ligand-dependent heterodimerization between HER2 and another HER family member, HER1, HER3 or HER4, activates the HER2 signaling pathway. The intracellular signaling pathway of HER2 is thought to involve ras-MAPK and PI3K pathways, as well as MAPK-independent S6 kinase and phospholipase C-gamma signaling pathways. HER2 signaling also effects proangiogenic factors, vascular endothelial growth factor (VEGF) and interleukin-8 (IL-8), and an antiangiogenic factor, thrombospondin-1 (TSP-1).

The full-length ErbB2 receptor undergoes proteolytic cleavage releasing its extracellular domain (ECD), which can be detected in cell culture medium and in patient's sera. The truncated ErbB2 receptor (p95ErbB2) that remains after proteolytic cleavage exhibits increased autokinase activity and transforming efficiency compared with the full-length receptor, implicating the ErbB2 ECD as a negative regulator of ErbB2 kinase and oncogenic activity.

A recombinant humanized version of the murine anti-ErbB2 antibody 4D5 (huMAb4D5-8, rhuMAb HER2 or HERCEPTIN®; U.S. Pat. No. 5,821,337) is clinically active in patients with ErbB2-overexpressing metastatic breast cancers that have received extensive prior anti-cancer therapy (Baselga et al., J. Clin. Oncol. 14:737-744 (1996)). HERCEPTIN® reportedly targets the C-terminal region of domain IV of ErbB2. HERCEPTIN® clinical activity is predominately dependent on antibody dependent cell mediated cytotoxicity (ADCC). Studies have suggested that HERCEPTIN® acts by triggering G1 cell cycle arrest.

Presently ErbB-directed therapeutics do not meet the current medical needs. ErbB-directed therapeutics have had only modest anti-tumor efficacy and are not as potent as anticipated from preclinical models. In most patients who initially respond to HERCEPTIN®, disease progression is noted within 1 year. In the metastatic setting, a median duration of roughly nine months was reported, at which point it appears that patients frequently become refractory to therapy. Studies have suggested that more complete blockade of the ErbB receptor family would be beneficial. As there are multiple functional domains of HER2, agents targeted to each of the domains could be a potentially valuable therapeutic. Additionally, there are harmful side effects of HERCEPTIN® treatment. Cardiac dysfunction, quantitated as a decrease in left ventricular ejection fraction (LVEF) of 10% from baseline or less than 50% total, was identified in roughly 7.1% of patients receiving HERCEPTIN® for 1 year versus 2.2% in patients randomized to observation in the HERA trial. Rates of severe and symptomatic congestive heart failure (CHF) were also significantly higher in the group randomized to HERCEPTIN®. Potentially, agents targeting a different HER2 epitopes could avoid these side effects. Accordingly, there remains an urgent need for agents targeting HER2.

The EGFR family of receptor tyrosine kinases are important regulators of cell growth and proliferation. One member of the family, ErbB2, has been implicated in a host of disorders and diseases including many forms of cancer.

Accordingly, there is an urgent need for therapeutic and diagnostic agents for detecting and treating ErbB2-mediated disorders including proliferative disorders.

### SUMMARY OF THE INVENTION

The invention relates to novel ErbB2 binding proteins that bind the extracellular domain (ECD) of ErbB2, in particular, human ErbB2. The novel binding protein can be antibody, an antigen-binding fragment of an antibody or a small modular immunopharmaceutical (SMIP). In various embodiments, the binding proteins: bind the ECD in the L1, CR1, L2 or CR2 domain, in some cases in the membrane proximal region of the CR2 domain, such as a membrane proximal region comprising the amino acid sequence shown in the first 12 residues of SEQ ID NO: 671 (i.e., without the EKK). In some embodiments, a HER2 binding protein of the invention is an ErbB2 agonist, increases tyrosine phosphorylation of ErbB2 and/or of AKT, MAP kinase (MAPK), MEK kinase, ERK 1/2, preferentially binds ErbB2 ECD homodimer over monomer or shed ECD, binds HER2 on cells and in some cases internalizes, decreases shedding of ErbB2 ectodomain shedding compared to shedding from cells of the same type without a bound HER2 binding protein of the invention, reduces the amount of cell surface HER2, reduces ErbB2 mediated proliferation of cancer cells, increases apoptosis in cancer cells, increases the number of cells in S phase after treatment with the binding protein, reduces tumor growth in vivo, enhances the effectiveness of some other anti-proliferative or cytotoxic agents or any combination of these properties.

The invention further relates to nucleic acids encoding the binding proteins or their components, vectors and host cells comprising the nucleic acids and methods of producing the binding proteins by expressing them in the host cells.

In a further aspect, the invention provides kits and compositions comprising one or more binding proteins of the invention and in some embodiments, further comprising an additional component that is a therapeutic or diagnostic agent, particularly a chemotherapeutic agent.

The invention also provides methods for producing and identifying binding proteins of the invention and methods for using them, including for treating cancer or other ErbB2 mediated disorders in a subject in need thereof, for reducing proliferation of and/or increasing apoptosis in ErbB2 expressing cells, including cancer cells, for reducing tumor growth and for diagnostic uses, including detecting and/or quantifying the presence of ErbB2 or cells expressing it.

### BRIEF DESCRIPTION OF THE FIGURES

FIG. 1. Schematic representation of the selection strategy used in the generation of human anti-Her2 scFv binding domains.

FIG. 2 (A-M). Alignments of the heavy chain amino acid sequences of human anti-Her2 scFvs with the germline human V_{H} gene sequence. CDRs are in bold type.

FIG. 3 (A-L). Alignments of the light chain amino acid sequences of human anti-Her2 scFvs with the germline human V_{K} or V_{λ} sequence. CDRs are in bold type.

FIG. 4. (A) Schematic diagram of the protein constructs used for selection and screening of scFvs and SMIPs that bind to the extracellular domain of Her2. (B) scFvs and SMIPs are binned into 4 distinct groups according to their binding phenotype as determined using the reagents in FIG. 4A. (* Herceptin contact sites)

FIG. 5. ELISA data for scFv binding to Her2. Binding data for phage-expressed scFv binding to Her2-expressing cells is shown on the left side of the table and data for soluble scFv binding to purified Her2 proteins is shown on the right. ELISA data is scored using a range that correlates with binding signal as indicated by −, + etc.

FIG. 6. Binding of HER2SMIPs (HER067 and HER030), HERCEPTIN® (trastuzumab), and a trastuzumab SMIP (HER018) to (A) HER2 dimer; (B) HER2 monomer; and (C) HER2 shed ectodomain found in SKBR3 supernatant.

FIG. 7. ELISA and BIACORE® data for HERCEPTIN® (trastuzumab) and SMIPs binding to Her2. Graphs represent binding of HERCEPTIN® (trastuzumab), Her033 or Her030 binding to various Her2 proteins determined by standard ELISA methods. The table represents Kd values for HERCEPTIN® (trastuzumab), Her033, Her030 and Her018 (Herceptin SMIP) binding to various Her2 proteins as detected by BIACORE®.

FIG. 8 provides a summary of various specific SMIPs, HERCEPTIN® (trastuzumab), and a trastuzumab SMIP (HER018) binding to various HER2 molecules (different sizes and different species, including human, murine, and macaque) as well as binding to several different cancer cell lines.

FIGS. 9A-9H show cell surface binding of HER2SMIPs (HER067 and HER094), HERCEPTIN® (trastuzumab), and a trastuzumab SMIP (HER018) to cell lines (A) Ramos (Her2⁻/CD20⁺ control); (B) BT474; (C) 22rv1; (D) MDA-MB-175; (E) MDA-MB-361 (ATCC); (F) MDA-MB-453; (G) MDA-MB-361 (JL); and (H) SKBR3.

FIG. 10 provides a summary of the anti-proliferative activity of HER033 SMIP and HERCEPTIN® (trastuzumab) on several different cancer cell lines.

FIG. 11. Proliferation of MDA-MB-361 cells following treatment with HER030 or HER033. MDA-MB-361 (ATCC) breast cancer cells were plated in 96-well format and treated with 0-10 ug/ml anti-Her2 or control reagents for 72 hr. Cells were washed, fixed, and stained with DAPI. Stained nuclei were counted using Cellomics High Content assay measuring fluorescence at 360 nM.

FIG. 12 provides a summary of the anti-proliferative activity of various specific SMIPs, HERCEPTIN® (trastuzumab), and a trastuzumab SMIP (HER018) on several different cancer cell lines.

FIG. 13. Western blot analysis of effect of Her033 on Her2 receptor phosphorylation (Y1248) following 24 hr treatment of MDA-MB-361 breast cancer cells. Cells were treated in vitro with Her033, HERCEPTIN® (trastuzumab), or a small molecule Her2 kinase inhibitor for 24 hrs either alone or in the presence of heregulin (HRG1 10 ng/ml) activation of Her3. Protein lysates (50 ug/well) were size fractionated by SDS-PAGE, transferred to nitrocellulose and probed with anti-phospho-Her2(Y1248) antibody. Inhibition of the Her2 receptor kinase blocked the endogenous Her2 autophosphorylation at tyrosine 1248 relative to control. Treatment with Herceptin did not significantly modulate receptor phosphorylation whereas treatment with Her033 stimulated Her2 receptor phosphorylation. Western blots were subsequently reprobed with anti-Actin antibody as protein loading control.

FIG. 14. Her033 increases downstream phosphoprotein signal transduction in MDA-MB-361 and BT474 breast cancer cells. Cells were plated in 96-well format and treated with anti-Her2 reagents or Heregulin for 10 minutes. Cells were stained with either rabbit anti-pAKT, anti-pERK, anti-pS6K, or anti-p38MAPK antibodies and ALEXA594 labeled secondary antibody and cellular fluorescence quantified by high content (Cellomics) analysis. In both breast cancer cell lines, treatment with Her033 SMIP induces phosphorylation of AKT and ERK proteins similar to treatment with the Her3 ligand Heregulin. MDA-MB-361 cells also demonstrate significant activation of p38MAP kinase.

FIG. 15. Kinetic analysis of Her033 stimulated downstream effector phosphorylation in MDA-MB-361 breast cancer cells. Cells were grown in 96-well format and treated with either anti-Her2 reagents or Her3 ligand Heregulin for 10 min to 24 hr as indicated. Cells were stained with either rabbit anti-pAKT, anti-pERK, anti-pS6K, or anti-p38MAPK antibodies and ALEXA594 labeled secondary antibody and cellular fluorescence quantified by high content (Cellomics) analysis. Her033 treatment induces sustained activation of AKT, ERK and p38MAP kinase phosphorylation in this cell line similar in magnitude to levels following stimulation with 10 ng/ml Heregulin.

FIGS. 16A and 16B show level of phosphorylation of ErbB2, and ERK1/2 in MDA-MB-361 cells when treated with HER2SMIP HER067, HERCEPTIN® (trastuzumab), and a trastuzumab SMIP (HER018).

FIG. 17 shows the effect on cell cycle of HER033SMIP, HERCEPTIN® (trastuzumab), and heregulin on the SKBR3 and BT474 cell lines.

FIG. 18 shows the effect on cell cycle of HER033SMIP, HERCEPTIN® (trastuzumab), and heregulin on the MDA-MB-453 and MDA-MB-361 cell lines.

FIG. 19. MDA-MB-361 xenograft progression in irradiated nu/nu mice. Female nu/nu mice were exposed to 400 rads of total body irradiation. After three days, they were injected subcutaneously in the dorsal right flank with 1×10⁷ MDA-MB-361 cells in Matrigel. When the tumors had reached a mass of 0.1-0.25 g, animals were dosed with Herceptin, HER033, or vehicle (100 ug/mouse, intraperitoneally) on days 1, 4, 6, 8 and 11 (n=10 mice/treatment group). Tumors were measured, and calculated tumor volumes for individual mice are shown for animals treated with vehicle (A), Herceptin (B), or HER033 (C). Animals developing tumors larger than 2.5 g were sacrificed. The mean tumor volume±SEM are plotted in (D). Means were not calculated for treatment groups in which animals with large tumors had been sacrificed.

FIG. 20. MDA-MB-361 xenograft progression in Balb/c nude mice. Male Balb/c nude mice were injected subcutaneously in the dorsal right flank with 1×10⁷ MDA-MB-361 cells in Matrigel. When the tumors had reached a mass of 0.1-0.25 g, animals were dosed with HERCEPTIN® (trastuzumab), HER033, or vehicle (100 ug/mouse, intraperitoneally) on days 1, 4, 6, 8 and 11 (n=10 mice/treatment group). Tumors were measured, and calculated tumor volumes for individual mice are shown for animals treated with vehicle (A), HERCEPTIN® (trastuzumab) (B), or HER033 (C). Animals developing tumors larger than 2.5 g were sacrificed. The mean tumor volume±SEM are plotted in (D). Means were not calculated for treatment groups in which animals with large tumors had been sacrificed.

FIGS. 21 and 22 show the in vivo efficacy of HER2SMIP HER033/HER067 when used to treat SCID-Beige having a tumor xenograft of MDA-MB-361 cells and the in vitro anti-proliferative activity on MDA-MB-361 cells. The top panel of FIG. 21 shows the mean tumor volume in mice treated with HER033SMIP, HERCEPTIN® (trastuzumab), or vehicle (IgG) after 21 days. The bottom panel of FIG. 21 shows a titration of anti-proliferative activity of HER2SMIPs (HER067 and HER094) and trastuzumab SMIP (HER018) on the MDA-MB-361 cells used for xenografting in the mice. FIG. 22 shows the tumor volume of individual mice in each treatment group.

FIG. 23 (A-M). Alignments of the heavy chain amino acid sequences of human anti-ERBB2 antibodies with the germline human V_{H} gene sequence. CDRs are in bold type.

FIG. 24 (A-M). Alignments of the light chain amino acid sequences of human anti-ERBB2 antibodies with the germline human V_{K} or V_{λ} sequence. CDRs are in bold type.

FIGS. 25A and 25B. FIG. 25A is a schematic representation of the “stumpy” strategy used in the generation of human anti-ERBB2 antibodies. FIG. 25B shows the predicted structure of the “stumpy peptide” used for selection. The EKK sequence at C terminus maintains the helical structure predicted from the NMR (Goetz et al., 2001. Biochemistry 40: 6534-6540).

FIG. 26 (A-K). Alignments of the heavy chain and light chain amino acid sequences of human anti-ERBB2 antibodies with the germline human V_{H} gene sequence. CDRs are in bold type. The human anti-ERBB2 antibodies were selected using the “stumpy” strategy.

FIG. 27 shows various HER2 soluble protein constructs used to investigate binding of molecules of the invention.

FIG. 28 provides a summary of various specific SMIPs, HERCEPTIN® (trastuzumab), and a trastuzumab SMIP (HER018) binding to various HER2 molecules (different sizes and different species, including human, murine, and macaque) as well as binding to Her2 monomers and shed extracellular domain.

FIG. 29 is a graphical representation of different SMIPs binding to various Her2 molecules.

FIG. 30 graphically depicts the binding of anti-HER2 “stumpy” binders (HER085, HER156 and HER 169) to soluble HER2 constructs.

FIG. 31 summarizes the cell surface binding of various HER2SMIPs to different cell lines.

FIG. 32 is a bar graph showing cell staining of JIMT-1 cells with severalanti-HER2SMIPS including “stumpy” binders.

FIG. 33 graphically depicts staining of various cell lines with HER146, HER156 and HER169.

FIG. 34 summarizes the cross-reactivity of various HER2SMIPs to Macaca Her2 and Murine Her2.

FIG. 35 shows BIACORE® data for HERCEPTIN® (trastuzumab) and SMIPs binding to soluble Her2 proteins.

FIG. 36 shows a titration of anti-proliferative activity of HER2SMIPs (Her147, Her102, Her124, Her067, Her146, Her116, Her094, and Her133), trastuzumab SMIP (HER018) and Herceptin on MDAMB361 (ATCC) cells.

FIG. 37 shows a titration of anti-proliferative activity of HER2SMIPs (Her146, Her067, Her094, and Her116), trastuzumab SMIP (HER018) and Herceptin on MDAMB361 (JL) cells.

FIG. 38 is a graph showing decreased proliferation of: MDA_MB-361 cells by anti-HER2SMIPS HER146 and HER116.

FIG. 39 is a table summarizing the anti-proliferative activity of various specific SMIPs, HERCEPTIN® (trastuzumab), and trastuzumab SMIP (HER018) on several different cancer cell lines.

FIG. 40 is a graph showing the effect of MEK kinase inhibitor (CL-1040) on anti-HER2SMIP anti-proliferative activity in MDA-MB-361 ATCC breast cancer cells.

FIG. 41 is a graph showing the effect of ERK1/2 kinase inhibitor (FR180204) on anti-HER2SMIP anti-proliferative activity in MDA-MB-361 ATCC breast cancer cells.

FIG. 42 is a graph showing the effect of ERK1 or ERK2 knockdown by RNA interference on anti-HER2SMIP anti-proliferative activity in MDA-MB-361 ATCC breast cancer cells.

FIG. 43 is an image of a Western blot showing the presence of phosphorylated HER2 at 24 hrs and 48 hrs after treatment of MDA-MB-361 ATCC breast cancer cells with HER033SMIP or HER146SMIP.

FIGS. 44A and 44B show the effect on cell cycle of various SMIPs on the (A) SKBR3 (24 hours) and (B) BT474 (24 hours) cell lines. Samples in bold are statistically higher than the controls. Samples followed by “**” are statistically lower than the controls (student T test with an error rate of 0.05).

FIGS. 45A-E show the effect on cell cycle of various SMIPs (A) MDA-MB-453 (24 hours), (B) MDA-MB-361 (JL) (24 hours), (C) MDA-MB-361 (JL) (48 hours), (D) MDA-MB-361 (ATCC) (24 hours), (E), and MDA-MB-361 (ATCC) (48 hours). Samples in bold are statistically higher than the controls. Samples followed by “**” are statistically lower than the controls (student T test with an error rate of 0.05).

FIG. 46 is a graph of the mean tumor volume over time after treatment in vivo with anti-HER2SMIPs HER146 and HER116 in SCID-Beige mice having an MDA-MB-361 (JL) cells tumor xenograft. HERCEPTIN® (trastuzumab) and vehicle (IgG) are positive and negative controls, respectively

FIG. 47 presents results in SCID-Beige mice having a tumor xenograft of MDA-MB-361 (JL) cells following treatment with HER146SMIP and HER116SMIP. The left panel shows the survival of mice treated with HER146SMIP, HER116SMIP, HERCEPTIN® (trastuzumab), or vehicle (IgG) over a timecourse of 60 days. The right panel shows tumor free progression of mice treated with HER146SMIP, HER116SMIP, HERCEPTIN® (trastuzumab), or vehicle (IgG) over a timecourse of 60 days. The chart at the bottom demonstrates the mean survival time of mice used in the study.

FIGS. 48A-D are a set of graphs of MDA-MB-361 xenograft tumor size in Balb/C nude mice after treatment with anti-HER2SMIP HER146. HERCEPTIN® (trastuzumab) and vehicle (IgG) are positive and negative controls, respectively. (A) summary of data from 10 mice in each treatment group; (B) data for individual mice in vehicle (negative control) group; (C) data for individual mice in HER146 treatment group; (D) data for individual mice in HERCEPTIN® (positive control) group.

FIGS. 49A-D are a set of graphs of MDA-MB-361 xenograft tumor size in irradiated nu/nu mice after treatment with anti-HER2SMIP HER146. HERCEPTIN® (trastuzumab) and vehicle (IgG) are positive and negative controls, respectively. (A) summary of data from 10 mice in each treatment group; (B) data for individual mice in vehicle (negative control) group; (C) data for individual mice in HER146 treatment group; (D) data for individual mice in HERCEPTIN® (positive control) group.

FIG. 50 presents data from two independent experiments investigating the effect of anti-HER2SMIPS of the invention on the shedding of HER2 ectodomain and on HER2 cell surface expression. (A) and (B) present the relative effect of various anti-HER2 SMIPS on ECD shedding as detected by ELISA. Panels (C) and (D) presents the relative effect of various anti-HER2SMIPS on HER2 expression.

FIG. 51 presents data from the anti-HER2SMIP cross-blocking experiments. (A) HERCEPTIN®; (B) HER018; (C) HER067; (D) HER094; (E) HER102; (F) HER116; (G) HER146; (H) RITUXAN® and anti-CD20 SMIP (negative control).

FIG. 52 is a chart summarizing the cross-blocking results.

FIG. 53 provide photographs depicting the internalization of anti-HER2 SMIP (panels A and B) and cell surface HER2 (panel C).

FIG. 54 is a graph depicting Fc dependent cellular cytoxicity (FcDCC) of various anti-HER2SMIPS in MDA-MB-361-JL and SKBR3 cells.

FIG. 55 is a graph depicting complement-dependent cytotoxicity (CDC) (complement-dependent cytotoxicity) in SKBR3 cells.

FIG. 56 presents data from ELISA testing of SMIP binding to Her2-SIIS after storage of the SMIP in plasma at various temperatures and durations. (A) Her067; (B) Her146.

FIG. 57 depict different possible ratios of SMIP/receptor complexes with their predicted mass.

FIG. 58 shows the masses of SMIP/receptor complexes observed following SEC-RI-MALLS analysis.

FIGS. 59A-D provide a series of dose response curves of different cells pre-treated with 5-fold dilution series of HER146 and then treated with corresponding 5-fold dilution series of different chemotherapeutic agents, or combinations thereof, and charts of the dilution series times of incubation used. (A) MDA-MB-453 cells with HER146 and Cisplatin or Taxol; (B) MDA-MB-453 cells with HER146 and Doxorubicin; (C) MDA-MB-361-JL cells with Cisplatin or Taxol; (D) MDA-MB-361-JLcells with HER146 and Doxorubicin or Gemcitabine.

FIG. 60 is an immunoblot with short (left) or long (right) exposures showing Her2 immunoprecipitated from Ramos or SKBR3 cell lysates by Herceptin, 3B5, HER156, or HER169.

FIG. 61 is two immunoblots in color and a black-and-white exposure of the color blot on the right, showing Her2 immunoprecipitated from Ramos, JIMT-1, or MDA-MB-361 ATCC cell lysates by human IgG, 3B5, HER116, HER156, or HER169.

### DETAILED DESCRIPTION OF THE INVENTION

### I. Definitions

In order that the present invention may be more readily understood, certain terms are first defined. Additional definitions are set forth throughout the detailed description. The present invention provides novel binding proteins that, specifically bind the extra cellular domain (ECD) of ErbB2, especially human ErbB2. In some embodiments, the binding protein is an antibody or an antigen binding fragment of such antibody that specifically binds the ECD. In other embodiments, the binding protein is a small modular immunopharmaceutical (SMIP).

The term “antibody” refers to an intact four-chain molecule having 2 heavy chains and 2 light chains, each heavy chain and light chain having a variable domain and a constant domain, or an antigen-binding fragment thereof, and encompasses any antigen-binding domain. In various embodiments, an antibody of the invention may be polyclonal, monoclonal, monospecific, polyspecific, bi-specific, humanized, human, chimeric, synthetic, recombinant, hybrid, mutated, grafted (including CDR grafted), or an in vitro generated antibody.

The term “antigen-binding fragment” of an antibody that specifically binds the ECD of ErbB2 refers to a portion or portions of the antibody that specifically binds to the ECD. An antigen-binding fragment may comprise all or a portion of an antibody light chain variable region (V_{L}) and/or all or a portion of an antibody heavy chain variable region (V_{H}) so long as the portion or portions are antigen-binding. However, it does not have to comprise both. Fd fragments, for example, have two V_{H} regions and often retain some antigen-binding function of the intact antigen-binding domain. Examples of antigen-binding fragments of an antibody include (1) a Fab fragment, a monovalent fragment having the V_{L}, V_{H}, C_{L} and C_{H}1 domains; (2) a F(ab′)₂ fragment, a bivalent fragment having two Fab fragments linked by a disulfide bridge at the hinge region; (3) a Fd fragment having the two V_{H} and C_{H}1 domains; (4) a Fv fragment having the V_{L} and V_{H} domains of a single arm of an antibody, (5) a dAb fragment (Ward et al., (1989) Nature 341:544-546), that has a V_{H} domain; (6) an isolated complementarity determining region (CDR), and (7) a single chain Fv (scFv). Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). These antibody fragments are obtained using conventional techniques known to those with skill in the art, and the fragments are evaluated for function in the same manner as are intact antibodies.

The term “effective amount” refers to a dosage or amount that is sufficient to alter ErbB2 activity, to ameliorate clinical symptoms or achieve a desired biological outcome, e.g., decreased cell growth or proliferation, decreased heterodimerization with another member of the EGF family decreased homodimerization, decrease tumor growth rate or tumor size, increased cell death etc.

The term “human antibody” includes antibodies having variable and constant region sequences corresponding substantially to human germline immunoglobulin sequences known in the art, including, for example, those described by Kabat et al. (See Kabat, et al. (1991) Sequences of Proteins of Immunological Interest, Fifth Edition, U.S. Department of Health and Human Services, NIH Publication No. 91-3242). The amino acid sequences of a human antibody, when aligned with germline immunoglobulin sequences, most closely align with human immunoglobulin sequences. The human antibodies of the invention may include amino acid residues not encoded by human germline immunoglobulin sequences (e.g., mutations introduced by random or site-specific mutagenesis in vitro or by somatic mutation in vivo). Such non-germline residues may occur in a framework region, a CDR, for example in the CDR3, or in the constant region. A human antibody can have one or more residues, such as any number from 1-15, including all of the integers between 1 and 15, or more, replaced with an amino acid residue that is not encoded by the human germline immunoglobulin sequence. CDRs are as defined by Kabat or in Chothia C, Lesk A M, Canonical structures for the hypervariable regions of immunoglobulins, J Mol Biol. 1987 Aug. 20; 196(4):901-17.

The phrase “inhibit” or “antagonize” an ErbB2/HER2 activity refers to a reduction, inhibition, or otherwise diminution of at least one activity of ErbB2 due to binding an anti-ErbB2 antibody or antigen binding portion, wherein the reduction is relative to the activity of ErbB2 in the absence of the same antibody or antigen-binding portion. The activity can be measured using any technique known in the art, including, for example, as described in the Examples. Activation of the Her2 receptor tyrosine kinase can be measured by the degree of phosphorylation of key tyrosine residues in the intracellular domain. For example, Tyr1248 is a known site of autophosphorylation and thus is a direct measure of Her2 receptor kinase activity. Typically the degree of phosphorylation can be determined by Western blot analysis probing with anti-phopho-Her2 specific antibodies (eg. Tyr1248, Tyr1139, Tyr1112, Tyr877, Tyr1221/1222). Alternatively, cells can be permeabilized and probed with fluorescently labeled phospho-Her2 antibodies and measured either by flow cytometry or high content (Cellomics) analysis. Additionally, the Her2 receptor can be immunoprecipitated, digested with trypsin protease and the degree of phosphorylation at specific sites within the individual Her2 peptides determined by standard Mass Spec techniques. Inhibition or antagonism does not necessarily indicate a total elimination of the ErbB2 polypeptide biological activity. In some embodiments, the reduction in activity may be about 10%, 20%, 30%, 40%, 50%, 60%, 70%, 75%, 80%, 85%, 90%, 95% or more, including 100% reduction, i.e., elimination of the activity.

The term “ErbB2” refers to erythroblastic leukemia viral oncogene homolog 2. In the case of human ErbB2, it also is known as c-erb-B2 or HER2/neu. In some embodiments the ErbB2 may comprise: (1) an amino acid sequence of a naturally occurring mammalian ErbB2 polypeptide (full length or mature form) or a fragment thereof, or a fragment thereof; (2) an amino acid sequence substantially identical to, e.g., at least 85%, 90%, 95%, 96%, 97%, 98%, 99% identical to said amino acid sequence or a fragment thereof; (3) an amino acid sequence that is encoded by a naturally occurring mammalian ErbB2 nucleotide sequence or a fragment thereof, or (4) a nucleotide sequence that hybridizes to the foregoing nucleotide sequence under stringent conditions, e.g., highly stringent conditions.

HER2 or c-erb-B2 encodes a transmembrane receptor protein of 185 kDa, which is structurally related to the epidermal growth factor receptor1. HER2 protein overexpression is observed in 25%-30% of primary breast cancers and is associated with decreased overall survival and a lowered response to chemotherapy and hormonal therapy, which can continue throughout the course of the disease and drives aggressive tumor growth.

The term “ErbB2 activity” refers to at least one cellular process initiated or interrupted as a result of ErbB2 binding to a receptor complex comprising ErbB2 and an ErbB receptor family member including ErbB1 (EGFR), ErbB2, ErbB3, ErbB4 or comprising an ErbB ligand such as but not limited to EGF, TGF-alpha, amphiregulin, betacellulin, heparin-binding EGF-like growth factor, GP30 on the cell. ErbB2 activity can be determined using any suitable assay methods, for example, protein overexpression can be determined using immunohistochemistry (1HC) and may also be inferred when HER2 gene amplification is identified using fluorescence in situ hybridization (FISH).

As used herein, “in vitro generated antibody” refers to an antibody where all or part of the variable region (e.g., at least one CDR) is generated in a non-immune cell selection (e.g., an in vitro phage display, protein chip or any other method in which candidate sequences can be tested for their ability to bind to an antigen). This term excludes sequences generated by genomic rearrangement in an immune cell.

The term “isolated” refers to a molecule that is substantially free of its natural environment. For instance, an isolated protein is substantially free of cellular material or other proteins from the cell or tissue source from which it was derived. The term also refers to preparations where the isolated protein is sufficiently pure for pharmaceutical compositions; or at least 70-80% (w/w) pure; or at least 80-90% (w/w) pure; or at least 90-95% pure; or at least 95%, 96%, 97%, 98%, 99%, or 100% (w/w) pure.

The phrase “percent identical” or “percent identity” refers to the similarity between at least two different sequences. This percent identity can be determined by standard alignment algorithms, for example, the Basic Local Alignment Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) that has been incorporated into the ALIGN program (version 2.0), using a PAM120 weight residue table, a gap length penalty of 12 and a gap penalty of 4. The percent identity is usually calculated by comparing sequences of similar length.

The terms “specific binding” or “specifically binds” refer to forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the association constant K_{A} is higher than 10⁶ M⁻¹. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques. An antibody is said to specifically bind an antigen when the K_{D} is ≦1 mM, preferably ≦100 nM.

As used herein, the term “stringent” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. One example of stringent hybridization conditions is hybridization in 6× sodium chloride/sodium citrate (SSC) at about 45° C., followed by at least one wash in 0.2×SSC, 0.1% SDS at 50° C. A second example of stringent hybridization conditions is hybridization in 6×SSC at about 45° C., followed by at least one wash in 0.2×SSC, 0.1% SDS at 55° C. Another example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. High stringent conditions include hybridization in 0.5M sodium phosphate, 7% SDS at 65° C., followed by at least one wash at 0.2×SSC, 1% SDS at 65° C.

The phrase “substantially as set out,” “substantially identical” or “substantially homologous” means that the relevant amino acid or nucleotide sequence (e.g., CDR(s), V_{H}, or V_{L} domain) will be identical to or have insubstantial differences (through conserved amino acid substitutions) in comparison to the sequences that are set out. Insubstantial differences include minor amino acid changes, such as 1 or 2 substitutions in a 5 amino acid sequence of a specified region. In the case of antibodies, the second antibody has the same specificity and has at least 50% of the affinity of the first antibody.

Sequences substantially identical or homologous (e.g., at least about 85% sequence identity) to the sequences disclosed herein are also part of this application. In some embodiment, the sequence identity can be about 85%, 90%, 95%, 96%, 97%, 98%, 99% or higher. Alternatively, substantial identity or homology exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

The term “therapeutic agent” is a substance that treats or assists in treating a medical disorder. Therapeutic agents may include, but are not limited to, anti-proliferative agents, anti-cancer agents including chemotherapeutics, anti-virals, anti-infectives, immune modulators, and the like that modulate immune cells or immune responses in a manner that complements the ErbB2 activity of an anti-ErbB2 binding protein of the invention. Non-limiting examples and uses of therapeutic agents are described herein.

As used herein, a “therapeutically effective amount” of an anti-ErbB2 binding protein refers to an amount of an binding protein that is effective, upon single or multiple dose administration to a subject (such as a human patient) at treating, preventing, curing, delaying, reducing the severity of, and/or ameliorating at least one symptom of a disorder or recurring disorder, or prolonging the survival of the subject beyond that expected in the absence of such treatment.

The term “treatment” refers to a therapeutic or preventative measure. The treatment may be administered to a subject having a medical disorder or who ultimately may acquire the disorder, in order to prevent, cure, delay, reduce the severity of, and/or ameliorate one or more symptoms of a disorder or recurring disorder, or in order to prolong the survival of a subject beyond that expected in the absence of such treatment.

### II. Anti-ErbB2 Binding Proteins

In a first aspect, the invention provides novel ErbB2/HER2, particularly human ErbB2/HER2, ErbB2/HER2 binding proteins that bind in the extra-cellular domain (ECD). In various embodiments, the binding proteins of the invention bind in the LR1, CR1, LR2 or CR2 domain of the ECD, including a membrane proximal region of CR2 comprising the amino acid sequence in the first twelve residues of SEQ ID NO: 671 (i.e., without the EKK). Unlike HERCEPTIN®, in some embodiments the binding proteins of the invention preferentially bind ErbB2 nomodimers over monomers or shed ECD. In some embodiments, the binding proteins of the invention bind ECD homodimers substantially more than monomers. In some cases, the binding protein has no appreciable or significant binding to ECD monomers or to shed ECD.

In some embodiments, the novel binding proteins are ErbB2 agonists and increase tyrosine phosphorylation of ErbB2 and at the same time, have anti-proliferative activity and pro-apoptotic activity. In some embodiments, the binding protein increases kinase activity in a HER-2 expressing cell, including but not limited to increasing kinase activity of MEK, MAPK, ERK1, ERK2 or a combination thereof.

The anti-ErbB2/HER2 binding proteins of the invention can be obtained by any of numerous methods known to those skilled in the art. For example, antibodies can be produced using recombinant DNA methods (U.S. Pat. No. 4,816,567). Monoclonal antibodies may be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597WO 92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

The subunit structures, e.g., a C_{H}, V_{H}, C_{L}, V_{L}, CDR, FR, and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that a complete 4-chain immunoglobulin comprises active portions, e.g., a portion of the V_{H} or V_{L} domain or a CDR that binds to the antigen, i.e., an antigen-binding fragment, or, e.g., the portion of the C_{H} subunit that binds to and/or activates, e.g., an Fc receptor and/or complement. CDRs typically refer to regions that are hypervariable in sequence and/or form structurally defined loops, for example, Kabat CDRs are based on sequence variability, as described in Sequences of Proteins of Immunological Interest, US Department of Health and Human Services (1991), eds. Kabat et al, or alternatively, to the location of the hypervariable structural loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modelling software, which defines the contact hypervariable regions based on crystal structure. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Embodiments described with respect to Kabat CDRs can alternatively be implemented using similar described relationships with respect to Chothia hypervariable loops or to the AbM-defined loops.

In another embodiment, a monoclonal antibody is obtained from the non-human animal, and then modified, e.g., humanized, deimmunized, chimeric, may be produced using recombinant DNA techniques known in the art. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982), and may be made according to the teachings of PCT Publication WO92/06193 or EP 0239400).

An antibody or fragment thereof may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class 11 binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson, et al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

In certain embodiments, an antibody can contain an altered immunoglobulin constant or Fc region. For example, an antibody produced in accordance with the teachings herein may bind more strongly or with more specificity to effector molecules such as complement and/or Fc receptors, which can control several immune functions of the antibody such as effector cell activity, lysis, complement-mediated activity, antibody clearance, and antibody half-life. Typical Fc receptors that bind to an Fc region of an antibody (e.g., an IgG antibody) include, but are not limited to, receptors of the FcγRI, FcγRII, and FcγRIII and FcRn subclasses, including allelic variants and alternatively spliced forms of these receptors. Fc receptors are reviewed in Ravetch and Kinet, Annu. Rev. Immunol 9:457-92, 1991; Capel et al., Immunomethods 4:25-34, 1994; and de Haas et al., J. Lab. Clin. Med. 126:330-41, 1995).

For additional antibody production techniques, see Antibodies: A Laboratory Manual, eds. Harlow et al., Cold Spring Harbor Laboratory, 1988. The present invention is not necessarily limited to any particular source, method of production, or other special characteristics of an antibody.

In some embodiments, an anti-ErbB2 antibody of the invention may be a V_{HH} molecule. V_{HH} molecules (or nanobodies), as known to the skilled artisan, are heavy chain variable domains derived from immunoglobulins naturally devoid of light chains, such as those derived from Camelidae as described in WO9404678, incorporated herein by reference. Such a V_{HH} molecule can be derived from antibodies raised in Camelidae species, for example in camel, llama, dromedary, alpaca and guanaco and is sometomes called a camelid or camelized variable domain. See e.g., Muyldermans., J. Biotechnology (2001) 74(4):277-302, incorporated herein by reference. Other species besides Camelidae may produce heavy chain antibodies naturally devoid of light chain. V_{HH} molecules are about 10 times smaller than IgG molecules. They are single polypeptides in which the CDR3 is longer than a conventional antibody, the VH:VL interface residues are different, and extra cysteines are generally present. These molecules tend to be very stable, resisting extreme pH and temperature conditions. Moreover, they are resistant to the action of proteases which is not the case for conventional antibodies. Furthermore, in vitro expression of V_{HH}s produces high yield, properly folded functional V_{HH}s. In addition, antibodies generated in Camelids will recognize epitopes other than those recognized by antibodies generated in vitro through the use of antibody libraries or via immunization of mammals other than Camelids (see WO 9749805, that is incorporated herein by reference). In additional embodiments, an anti-ErbB2 antibodies or binding fragments of the invention may include single domain antibodies such as immunoglobulin new antigen receptors (IgNARs), which are a unique group of antibody isotypes found in the serum of sharks (Greenberg et al., Nature 374: 168-173 (1995); Nuttall et al., Mol. Immunol., 38: 313-326. (2001)). These are bivalent molecules, targeting antigen through a single immunoglobulin variable domain (˜13 kDa) displaying two complementarity determining region (CDR) loops (Roux et al., Proc. Natl. Acad. Sci., 95: 11804-11809 (1998)) and having unusually long and structurally complex CDR3s, which display a high degree of variability (Greenberg et al., 1995).

Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. Each light chain includes an N terminal variable (V) domain (V_{L}) and a constant (C) domain (C_{L}). Each heavy chain includes an N terminal V domain (V_{H}), three or four C domains (C_{H}s), and a hinge region collectively referred to as the constant region of the heavy chain. The C_{H} domain most proximal to V_{H} is designated as C_{H}1. The V_{H} and V_{L} domains consist of four regions of relatively conserved sequences called framework regions (FR1, FR2, FR3, and FR4), that form a scaffold for three regions of hypervariable sequences also referred to as complementarity determining regions CDRs. CDRs are referred to as CDR1, CDR2, and CDR3. Accordingly, CDR constituents on the heavy chain may be referred to as HCDR1, HCDR2, and HCDR3, while CDR constituents on the light chain are referred to as LCDR1, LCDR2, and LCDR3. CDR3 is typically the greatest source of molecular diversity within the antibody-binding site.

The anti-ErbB2 binding proteins of the invention include complete 4-chain antibodies and antigen-binding fragments of complete antibodies. An antigen-binding fragment (also referred to as an antigen-binding portion) includes but is not limited to Fab, Fv and ScFv molecules. The Fab fragment (Fragment antigen-binding) consists of V_{H}-C_{H}1 and V_{L}-C_{L} domains covalently linked by a disulfide bond between the constant regions. The Fᵥ fragment is smaller and consists of V_{H} and V_{L} domains non-covalently linked. To overcome the tendency of non-covalently linked domains to dissociate, a single chain Fᵥ fragment (scFᵥ) can be constructed. The scFᵥ contains a flexible polypeptide that links (1) the C-terminus of V_{H} to the N-terminus of V_{L}, or (2) the C-terminus of V_{L} to the N-terminus of V_{H}. Repeating units of (Gly₄Ser)_often 3 or 4 repeats may be used as a linker, but other linkers are known in the art.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). In one embodiment, the bispecific antibody comprises a first binding domain polypeptide, such as a Fab′ fragment, linked via an immunoglobulin constant region to a second binding domain polypeptide.

In some embodiments, an anti-ErbB2 binding protein of the invention is a Small Modular ImmunoPharmaceuticals (SMIP™). SMIPs and their uses and applications are disclosed in, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

A SMIP™ typically refers to a binding domain-immunoglobulin fusion protein that includes a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than C_{H}1, for example, the C_{H}2 and C_{H}3 regions of IgG and IgA, or the C_{H}3 and C_{H}4 regions of IgE (see e.g., U.S. 2005/0136049 by Ledbetter, J. et al., which is incorporated by reference, for a more complete description). The binding domain-immunoglobulin fusion protein can further include a region that includes a native or engineered immunoglobulin heavy chain C_{H}2 constant region polypeptide (or C_{H}3 in the case of a construct derived in whole or in part from IgE) that is fused or otherwise connected to the hinge region polypeptide and a native or engineered immunoglobulin heavy chain C_{H}3 constant region polypeptide (or C_{H}4 in the case of a construct derived in whole or in part from IgE) that is fused or otherwise connected to the C_{H}2 constant region polypeptide (or C_{H}3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a target antigen, such as human ErbB2.

The binding domain of a SMIP of the invention may contain a complete V_{H} and a complete V_{L} joined by linker antigen-binding portions of a V_{H} and/or V_{L} and may V2 or be linked in either orientation, i.e., V_{H}-linker-V_{L} or V_{L}-linker-V_{H}. Any suitable linker can be used in a SMIP of the invention and will be known to those of skill in the art. Exemplary linkers may be found, for example in WO 2007/146968 Tables 5 and 10-12 of which are incorporated by reference in their entirety. Likewise, any immunoglobulin hinge sequence or hinge-acting sequence may be used in a SMIP of the invention.

In some SMIP embodiments at least one of the immunoglobulin heavy chain constant region polypeptides (i.e., CH2, CH3 or CH4) is from a human immunoglobulin heavy chain. In various embodiments, the immunoglobulin heavy chain constant region polypeptides are of an isotype selected from human IgG and human IgA. In certain further embodiments of the above described SMIP, the linker polypeptide comprises at least one polypeptide having as an amino acid sequence (Gly₄, Ser) and in certain other embodiments the linker polypeptide comprises at least three repeats of said polypeptide. In certain embodiments the immunoglobulin hinge region polypeptide comprises a human IgA hinge region polypeptide.

An immunoglobulin hinge region polypeptide, as discussed above, includes any hinge peptide or polypeptide that occurs naturally, as an artificial peptide or as the result of genetic engineering and that is situated in an immunoglobulin heavy chain polypeptide between the amino acid residues responsible for forming intrachain immunoglobulin-domain disulfide bonds in CH1 and CH2 regions; hinge region polypeptides for use in the present invention may also include a mutated hinge region polypeptide. Accordingly, an immunoglobulin hinge region polypeptide may be derived from, or may be a portion or fragment of (i.e., one or more amino acids in peptide linkage, typically 5-65 amino acids, preferably 10-50, more preferably 15-35, still more preferably 18-32, still more preferably 20-30, still more preferably 21, 22, 23, 24, 25, 26, 27, 28 or 29 amino acids) an immunoglobulin polypeptide chain region classically regarded as having hinge function, as described above. But, a hinge region polypeptide for use in the instant invention need not be so restricted and may include amino acids situated (according to structural criteria for assigning a particular residue to a particular domain that may vary, as known in the art) in an adjoining immunoglobulin domain such as a CH1 domain or a CH2 domain, or in the case of certain artificially engineered immunoglobulin constructs, an immunoglobulin variable region domain.

Wild-type immunoglobulin hinge region polypeptides include any naturally occurring hinge region that is located between the constant region domains, CH1 and CH2, of an immunoglobulin. The wild-type immunoglobulin hinge region polypeptide is preferably a human immunoglobulin hinge region polypeptide, preferably comprising a hinge region from a human IgG immunoglobulin, and more preferably, a hinge region polypeptide from a human IgG1 isotype. As is known to the art, despite the tremendous overall diversity in immunoglobulin amino acid sequences, immunoglobulin primary structure exhibits a high degree of sequence conservation in particular portions of immunoglobulin polypeptide chains, notably with regard to the occurrence of cysteine residues which, by virtue of their sulfyhydryl groups, offer the potential for disulfide bond formation with other available sulfydryl groups. Accordingly, in the context of the present invention wild-type immunoglobulin hinge region polypeptides may be regarded as those that feature one or more highly conserved (e.g., prevalent in a population in a statistically significant manner) cysteine residues, and in certain preferred embodiments a mutated hinge region polypeptide may be selected that contains zero or one cysteine residue and that is derived from such a wild-type hinge region.

A mutated immunoglobulin hinge region polypeptide may comprise a hinge region that has its origin in an immunoglobulin of a species, of an immunoglobulin isotype or class, or of an immunoglobulin subclass that is different from that of the CH2 and CH3 domains. For instance, in certain embodiments of the invention, the SMIP may comprise a binding domain polypeptide that is fused to an immunoglobulin hinge region polypeptide comprising a wild-type human IgA hinge region polypeptide, or a mutated human IgA hinge region polypeptide that contains zero or only one cysteine residues, as described herein. Such a hinge region polypeptide may be fused to an immunoglobulin heavy chain CH2 region polypeptide from a different Ig isotype or class, for example an IgG subclass, which in certain preferred embodiments will be the IgG1 subclass.

In some embodiments, an anti-ErbB2 antibody of the invention is a V_{HH} molecule. V_{HH} molecules (or nanobodies), as known to the skilled artisan, are heavy chain variable domains derived from immunoglobulins naturally devoid of light chains, such as those derived from Camelidae as described in WO9404678, incorporated herein by reference. Such a V_{HH} molecule can be derived from antibodies raised in Camelidae species, for example in camel, llama, dromedary, alpaca and guanaco and is sometomes called a camelid or camelized variable domain. See e.g., Muyldermans., J. Biotechnology (2001) 74(4):277-302, incorporated herein by reference. Other species besides Camelidae may produce heavy chain antibodies naturally devoid of light chain. V_{HH} molecules are about 10 times smaller than IgG molecules. They are single polypeptides and very stable, resisting extreme pH and temperature conditions. Moreover, they are resistant to the action of proteases which is not the case for conventional antibodies. Furthermore, in vitro expression of V_{HH}s produces high yield, properly folded functional V_{HH}s. In addition, antibodies generated in Camelids will recognize epitopes other than those recognized by antibodies generated in vitro through the use of antibody libraries or via immunization of mammals other than Camelids (see WO 9749805, that is incorporated herein by reference).

Amino acid (AA) sequences of illustrative heavy chain variable domains (V_{H}) and light chain variable domains (V_{L}) of the anti-ErbB2 antibodies of this invention, are set forth in the attached Sequence Table. Table 1 provides the Sequence Identifiers (SEQ ID Nos) of the V_{H} and V_{L} domains. One hundred specific embodiments of the antibodies are identified as: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1_R3A11DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R2_DP47_{—}1A10, S6R2_DP47_{—}1E11, S5R2_DP47_{—}1H1, S6R3_CS_{—}1G5, S6R2_DP47_{—}1H11, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9.

TABLE 1
HUMAN ANTI-ErbB2 BINDING DOMAINS

SEQUENCE IDENTIFIER
(SEQ ID Nos:)
Variable Domain Protein
Sequences

| scFv | Heavy | Light |
|---|---|---|
| S1R2A_CS_1F7 | 1 | 2 and 63 |
| S1R2A_CS_1D11 | 3 | 4 and 64 |
| S1R2C_CS_1D3 | 5 and 65 | 6 and 66 |
| S1R2C_CS_1H12 | 7 and 67 | 8 and 68 |
| S1R2A_CS_1D3 | 9 | 10 and 69 |
| S1R3B2_BMV_1E1 | 11 | 12 and 70 |
| S1R3C1_CS_1D3 | 13 | 14 and 71 |
| S1R3B2_DP47_1E8 | 15 | 16 and 72 |
| S1R3B2_BMV_1G2 | 17 | 18 and 73 |
| S1R3B2_BMV_1H5 | 19 | 20 and 74 |
| S1R3C1_CS_1A6 | 21 | 22 and 75 |
| S1R3B2_DP47_1C9 | 23 | 24 and 76 |
| S1R3B2_DP47_1E10 | 25 | 26 and 77 |
| S1R3C1_CS_1B10 | 27 | 28 and 78 |
| S1R3A1_BMV_1F3 | 29 | 30 and 79 |
| S1R3B1_BMV_1G11 | 31 | 32 and 80 |
| S1R3A1_BMV_1G4 | 33 | 34 and 81 |
| S1R3B1_BMV_1H11 | 35 | 36 and 82 |
| S1R3A1_CS_1B9 | 37 | 38 and 83 |
| S1R3B1_BMV_1H9 | 39 | 40 and 84 |
| S1R3A1_CS_1B10 | 41 | 42 and 85 |
| S1R3B1_BMV_1C12 | 43 | 44 and 86 |
| S1R3C1_BMV_1H11 | 45 | 46 and 87 |
| S1R3B1_BMV_1A10 | 47 | 48 and 88 |
| S1R3A1_CS_1D11 | 49 | 50 and 89 |
| S1R3C1_DP47_1H1 | 51 | 52 and 90 |
| S1R3A1_CS_1B12 | 53 | 54 and 91 |
| S1R3B1_BMV_1H5 | 55 | 56 and 92 |
| S1R3A1_DP47_1A6 | 57 | 58 and 93 |
| S1R3B1_DP47_1E1 | 59 | 60 and 94 |
| S1R3B1_BMV_1A1 | 61 | 62 and 95 |
| S1R3B1_DP47_3A2 | 251 | 253 |
| S1R3A1_DP47_11B7 | 255 | 257 |
| S1R3A1_DP47_11D1 | 259 | 261 |
| S1R3A1_DP47_7F3 | 263 | 265 |
| S1R2B_DP47_4E3 | 267 | 269 |
| S1R3C1_DP47_2G2 | 271 | 273 |
| S1R3A1_DP47_11H6 | 275 | 277 |
| S1R3A1_BMV_3B1 | 279 | 281 |
| S1R3A1_DP47_6B9 | 283 | 285 |
| S1R2A_CS_10B8 | 287 | 289 |
| S1R3A1_DP47_7A6 | 291 | 293 |
| S1R3B2_DP47_2G3 | 295 | 297 |
| S1R2B_CS_6H11 | 299 | 301 |
| S1R3A1_DP47_10G1 | 303 | 305 |
| S1R3A1_DP47_7C1 | 307 | 309 |
| S1R2A_DP47_5D6 | 311 | 313 |
| S1R3A1_DP47_11F6 | 315 | 317 |
| S1R3A1_DP47_11D3 | 319 | 321 |
| S1R3A1_CS_8A8 | 323 | 325 |
| S1R3A1_BMV_5D10 | 327 | 329 |
| S1R3A1_DP47_11C1 | 331 | 333 |
| S1R3A1_DP47_4E1 | 335 | 337 |
| S1R3A1_DP47_10E1 | 339 | 341 |
| S1R3A1_CS_11C3 | 343 | 345 |
| S1R3A1_CS_13H11 | 347 | 349 |
| S1R3A1_CS_2D9 | 351 | 353 |
| S1R2A_CS_3D4 | 355 | 357 |
| S1R3A1_DP47_2H6 | 359 | 361 |
| S1R3A1_DP47_4G1 | 363 | 365 |
| S1R2A_DP47_3C1 | 367 | 369 |
| S1R3A1_DP47_7B2 | 371 | 373 |
| S1R3B2_DP47_4E2 | 375 | 377 |
| S1R3A1_CS_16C2 | 379 | 381 |
| S1R3A1_CS_11E5 | 383 | 385 |
| S1R3A1_CS_16D7 | 387 | 389 |
| S1R2A_CS_10B10 | 391 | 393 |
| S1R3A1_CS_15C2 | 395 | 397 |
| S1R3A1_CS_9C1 | 399 | 401 |
| S1R2A_CS_5A1 | 403 | 405 |
| S1R2A_CS_8C8 | 407 | 409 |
| S1R3A1_CS_13H5 | 411 | 413 |
| S1R2B_CS_5E9 | 415 | 417 |
| S1R3A1_CS_8F9 | 419 | 421 |
| S1R3A1_CS_14B5 | 423 | 425 |
| S1R2A_CS_9E10 | 427 | 429 |
| S1R3A1_CS_7A10 | 431 | 433 |
| S1R3A1_BMV_6H7 | 435 | 437 |
| S1R3A1_CS_12A11 | 439 | 441 |
| S1R3A1_CS_13D12 | 443 | 445 |
| S1R3A1_CS_7A8 | 447 | 449 |
| S1R2A_CS_2C9 | 451 | 453 |
| S1R3A1_CS_12D1 | 455 | 457 |
| S1R2A_CS_7D4 | 459 | 461 |
| S1R3A1_CS_15B8 | 463 | 465 |
| S6R3_DP47_1A10 | 467 | 469 |
| S6R2_DP47_1E11 | 471 | 473 |
| S5R2_DP47_1H11 | 475 | 477 |
| S6R3_CS_1G5 | 479 | 481 |
| S6R2_DP47_1H11 | 483 | 485 |
| S5R3_DP47_1A10 | 487 | 489 |
| S5R2_DP47_1D11 | 491 | 493 |
| S5R2_CS_1A8 | 495 | 497 |
| S6R3_CS_1B7 | 499 | 501 |
| S6R2_CS_1E5 | 503 | 505 |
| S6R3_BMV_1C2 | 507 | 509 |
| S5R2_DP47_1B10 | 511 | 513 |
| S6R3_DP47_1C12 | 515 | 517 |
| S5R2_DP47_1D10 | 519 | 521 |
| S6R3_DP47_1H9 | 523 | 525 |

According to the nomenclature used herein, “S1R2A_CS_{—}1F7” indicates clone 1F7 from round 2A of the first selection from the CS library.

An anti-ErbB2 binding protein of this invention may optionally comprise antibody constant regions or parts thereof. For example, a V_{L} domain may be attached at its C-terminal end to a light chain constant domain which can be a Cκ or a Cλ. Similarly, a V_{H} domain or portion thereof may be attached to all or part of a heavy chain constant region, which can be a IgA, IgD, IgE, IgG, or IgM constant region or any isotype subclass including IgG1, IgG2, IgG3, IgG4, IgA1 or IgA2. Constant region sequences are known in the art (see, for example, Kabat et al., Sequences of Proteins of Immunological Interest, No. 91-3242, National Institutes of Health Publications, Bethesda, Md. (1991)). Therefore, binding proteins within the scope of this invention may include V_{H} and V_{L} domains, or a portion thereof, combined with constant regions or portions thereof known in the art.

In certain embodiments of the invention, the ErbB2 binding protein comprises a V_{H} domain, a V_{L} domain, or a combination thereof, comprising the V_{H} or V_{L} amino acid sequence, respectively, found in any one of S1R2A_CS_{—}1F7, S1R2A_CS_{—}1 D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H11, S6R3_CS_{—}1G5, S6R2_DP47_{—}1H11, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9. In some embodiments, the V_{H} and V_{L} are from the same reference antibody. That is, an anti-ErbB2 binding protein of the invention may comprise both the V_{H} and V_{L} amino acid sequence of one of the above-listed antibodies.

An anti-ErbB2 antibody of the invention may comprise one, two, three, four, five or all six complementarity determining regions (CDRs) from any one of the above-listed antibodies. In some embodiments, an anti-ErbB2 binding protein of the invention comprises the HCDR1, HCDR2 and HCDR3 (heavy chain CDR set), the LCDR1, LCDR2 and LCDR3 (light chain CDR set) or both the heavy chain CDR set and the light chain CDR set of one of the anti-ErbB2 antibodies exemplified herein.

A CDR3 sequence found in any one of the specifically exemplified anti-ErbB2 antibodies are encompassed within the scope of this invention. For example, in one embodiment, an anti-ErbB2 binding protein of the invention comprises an HCDR3 amino acid sequence found in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H1, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1CS_{—}16C2, S1R3A1CS_{—}11E5, S1R3A1CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H11, S6R3_CS_{—}1G5, S6R2_DP47_{—}1H11, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9.

In certain embodiments, the V_{H} and/or V_{L} domains may be germlined, i.e., the framework regions (FR) of these domains are mutated using conventional molecular biology techniques to match the germline sequence. In other embodiments, the FR sequences remain diverged from the consensus germline sequences.

In one embodiment, mutagenesis is used to make an antibody more similar to one or more germline sequences. This may be desirable when mutations are introduced into the framework region of an antibody through somatic mutagenesis or through error prone PCR. Germline sequences for the V_{H} and V_{L} domains can be identified by performing amino acid and nucleic acid sequence alignments against the VBASE database (MRC Center for Protein Engineering, UK). VBASE is a comprehensive directory of all human germline variable region sequences compiled from over a thousand published sequences, including those in the current releases of the Genbank and EMBL data libraries. In some embodiments, the FR regions of the scFvs are mutated in conformity with the closest matches in the VBASE database and the CDR portions are kept intact.

In certain embodiments, an anti-ErbB2 binding of this invention specifically binds the same epitope as, competes with or cross-competes with an antibody selected from the group consisting of: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D1, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H1, S6R3_CS_{—}1G5, S6R2_DP47_{—}1H1, S5R3_DP47_{—}1A10, S5R2_DP47_{—}1 D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9, for binding to ErbB2. In some embodiments, such competing or ErbB2-mediated cross-competing binding protein is an ErbB2 agonist and may further reduce proliferation of a cancer cell, reduce the rate of growth of an ErbB2-expressing tumor and/or increases apoptosis in such cells and tumors. In some embodiments, such competing or cross-competing binding proteins bind ErbB2 ECD homo-dimers but do not bind ECD monomers or shed ECD.

Such antibodies can be identified in a competitive binding assay. One can determine whether an antibody binds to the same epitope or cross competes for binding with a binding protein of the invention antibody by using methods known in the art. In one embodiment, one allows the binding protein of the invention to bind to ErbB2 under saturating conditions and then measures the ability of the test protein to bind to the ECD. If the test antibody is able to bind to the ECD at the same time as the reference binding protein, then the test antibody binds to a different epitope than the reference binding protein. However, if the test protein is not able to bind the to the ECD at the same time, then the test protein binds to the same epitope, an overlapping epitope, or an epitope that is in close proximity to the epitope bound by the binding protein of the invention. This experiment can be performed using ELISA, RIA, BIACORE™, or flow cytometry. To test whether a binding protein cross-competes with another anti-ErbB2 binding protein, one may use the competition method described above in two directions, i.e. determining if the known binder blocks the test binder and vice versa. In a preferred embodiment, the experiment is performed using BIACORE™.

In one embodiment, the association constant (K_{A}) of an ErbB2 binding protein of the invention is at least 10⁶ M⁻¹. In another embodiment, the association constant of these antibodies for human ErbB2 is at least 10⁹ M⁻¹. In other embodiments, the association constant of these antibodies for human ErbB2 is at least 10¹⁰ M⁻¹, at least 10¹¹ M⁻¹, or at least 10¹² M⁻¹. The binding affinity may be determined using techniques known in the art, such as ELISA, biosensor technology, such as biospecific interaction analysis, or other techniques including those described in this application.

In addition to sequence homology analyses, epitope mapping (see, e.g., Epitope Mapping Protocols, ed. Morris, Humana Press, 1996), and secondary and tertiary structure analyses can be carried out to identify specific 3D structures assumed by the presently disclosed antibodies and their complexes with antigens. Such methods include, but are not limited to, X-ray crystallography (Engstom (1974) Biochem. Exp. Biol., 11:7-13) and computer modeling of virtual representations of the present antibodies (Fletterick et al. (1986) Computer Graphics and Molecular Modeling, in Current Communications in Molecular Biology, Cold Spring Harbor Laboratory, Cold Spring Harbor, N.Y.).

The invention further provides anti-ErbB2 binding proteins that comprise altered V_{H} and/or V_{L} sequence(s) compared to the sequences in Table 1. Such binding proteins may be produced by a skilled artisan using techniques well-known in the art. For example, amino acid substitutions, deletions, or additions can be introduced in FR and/or CDR regions. FR changes are usually designed to improve the stability and immunogenicity of the antibody, while CDR changes are typically designed to increase antibody affinity for its antigen. The changes that increase affinity may be tested by altering CDR sequence and measuring antibody affinity for its target (see Antibody Engineering, 2nd ed., Oxford University Press, ed. Borrebaeck, 1995).

Antibodies whose CDR sequences differ insubstantially from those found in any one of specifically exemplified anti-ErbB2 antibodies are encompassed within the scope of this invention. Typically, this involves substitution of an amino acid with an amino acid having similar charge, hydrophobic, or stereochemical characteristics. More drastic substitutions in FR regions, in contrast to CDR regions, may also be made as long as they do not adversely affect (e.g., reduce affinity by more than 50% as compared to unsubstituted antibody) the binding properties of the binding protein. Substitutions may also be made to germline the binding protein or stabilize the antigen binding site.

Conservative modifications will produce molecules having functional and chemical characteristics similar to those of the molecule from which such modifications are made. In contrast, substantial modifications in the functional and/or chemical characteristics of the molecules may be accomplished by selecting substitutions in the amino acid sequence that differ significantly in their effect on maintaining (1) the structure of the molecular backbone in the area of the substitution, for example, as a sheet or helical conformation, (2) the charge or hydrophobicity of the molecule at the target site, or (3) the size of the molecule.

For example, a “conservative amino acid substitution” may involve a substitution of a native amino acid residue with a normative residue such that there is little or no effect on the polarity or charge of the amino acid residue at that position. (See, for example, MacLennan et al., 1998, Acta Physiol. Scand. Suppl. 643:55-67; Sasaki et al., 1998, Adv. Biophys. 35:1-24). Desired amino acid substitutions (whether conservative or non-conservative) can be determined by those skilled in the art at the time such substitutions are desired. For example, amino acid substitutions can be used to identify important residues of the molecule sequence, or to increase or decrease the affinity of the molecules described herein. Exemplary amino acid substitutions include, but are not limited to, those set forth in Table 2.

TABLE 2
Amino Acid Substitutions

| Original | Exemplary | More Conservative |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, Phe | Ile |
| Lys (K) | Arg, 1,4Diamino-butyric Acid, | Arg |
| | Gln, Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

In certain embodiments, conservative amino acid substitutions also encompass non-naturally occurring amino acid residues that are typically incorporated by chemical peptide synthesis rather than by synthesis in biological systems.

In one embodiment, the method for making a variant V_{H} domain comprises adding, deleting, or substituting at least one amino acid in the disclosed V_{H} domains, and testing the variant V_{H} domain for ErbB2 binding or modulation of ErbB2 activity.

An analogous method for making a variant V_{L} domain comprises adding, deleting, or substituting at least one amino acid in the disclosed V_{L} domains, and testing the variant V_{L} domain for ErbB2 binding or modulation of ErbB2 activity.

A further aspect of the invention provides a method for preparing antibodies or antigen-binding fragments that specifically bind ErbB2. The method comprises:

(a) providing a starting repertoire of nucleic acids encoding a V_{H} domain that lacks at least one CDR or contains at least one CDR to be replaced;

(b) inserting into or replacing the CDR region of the starting repertoire with at least one donor nucleic acid encoding an amino acid sequence as substantially set out herein for a V_{H} CDR, yielding a product repertoire;

(c) expressing the nucleic acids of the product repertoire;

(d) selecting a specific antigen-binding fragment that binds to ErbB2; and

(e) recovering the specific antigen-binding fragment or nucleic acid encoding it.

In an analogous method, at least one V_{L} CDR or V_{H} CDR of the invention is combined with a repertoire of nucleic acids encoding a V_{L} or V_{H} domain, respectively, that lacks at least one CDR or contains at least one CDR to be replaced. The at least one V_{H} or V_{L} CDR may be a CDR1, a CDR2, a CDR3, or a combination thereof, found in any of the specifically exemplified anti-ErbB2 antibodies.

In one embodiment, the variable domain includes a CDR3 to be replaced or lacks a CDR3 encoding region and the at least one donor nucleic acid encodes a CDR3 amino acid sequence found in any one of SEQ ID Nos:1-95, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 507, 509, 511, 513, 515, 517, 519, 521, 523, 525, or substantially as found in such sequence.

In another embodiment, the variable domain includes a CDR1 to be replaced or lacks a CDR1 encoding region and the at least one donor nucleic acid encodes a CDR1 amino acid sequence found in any one of SEQ ID Nos: 1-95, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 507, 509, 511, 513, 515, 517, 519, 521, 523, 525.

In another embodiment, the variable domain includes a CDR2 to be replaced or lacks a CDR2 encoding region and the at least one donor nucleic acid encodes a CDR2 amino acid sequence found in any one of SEQ ID Nos: 1-95, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 507, 509, 511, 513, 515, 517, 519, 521, 523, 525.

In another embodiment, the variable domain includes a CDR3 to be replaced or lacks a CDR3 encoding region and further comprises a CDR1 to be replaced or lacks a CDR1 encoding region, where the at least one donor nucleic acid encodes a CDR3a CDR1 amino acid sequence, respectively, found in any one of SEQ ID Nos: 1-95, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 507, 509, 511, 513, 515, 517, 519, 521, 523, 525.

In another embodiment, the variable domain includes a CDR3 to be replaced or lacks a CDR3 encoding region and further comprises a CDR2 to be replaced or lacks a CDR2 encoding region, where the at least one donor nucleic acid encodes a CDR3 or CDR2 amino acid sequence, respectively, found in any one of SEQ ID Nos: 1-95, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 507, 509, 511, 513, 515, 517, 519, 521, 523, 525.

In another embodiment, the variable domain includes a CDR3 to be replaced or lacks a CDR3 encoding region and further comprises a CDR1 and a CDR2 to be replaced or lacks a CDR1 and a CDR2 encoding region, where the at least one donor nucleic acid encodes CDR3, CDR1 or CDR2 amino acid sequence, respectively, found in any one of SEQ ID Nos: 1-95, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 507, 509, 511, 513, 515, 517, 519, 521, 523, 525.

The present invention further encompasses anti-ErbB2 antibodies comprising an HCDR3, an LCDR3 or both, three heavy chain CDRs, three light chain CDRs or all six CDRs, a V_{H} or V_{L} or an antigen-binding portion of such a V_{H} or V_{L} or both, of a specifically provided molecule herein

Using recombinant DNA methodology, a disclosed CDR sequence may be introduced into a repertoire of V_{H} or V_{L} domains lacking the respective CDR (Marks et al. (BioTechnology (1992) 10: 779-783). For example, a primer adjacent to the 5′ end of the variable domain and a primer to the third FR can be used to generate a repertoire of variable domain sequences lacking CDR3. This repertoire can be combined with a CDR3 of an antibody disclosed herein. Using analogous techniques, portions of a disclosed CDR sequence may be shuffled with portions of CDR sequences from other antibodies to provide a repertoire of antigen-binding fragments that bind ErbB2. Either repertoire can be expressed in a host system such as phage display (described in WO 92/01047 and its corresponding U.S. Pat. No. 5,969,108) so suitable antigen-binding fragments that bind to ErbB2 can be selected.

A further alternative uses random mutagenesis of a V_{H} or V_{L} sequence disclosed herein to generate variant V_{H} or V_{L} domains still capable of binding ErbB2. A technique using error-prone PCR is described by Gram et al. (Proc. Nat. Acad. Sci. U.S.A. (1992) 89: 3576-3580).

Another method uses direct mutagenesis of a V_{H} or V_{L} sequence disclosed herein. Such techniques are described by Barbas et al. (Proc. Nat. Acad. Sci. U.S.A. (1994) 91: 3809-3813) and Schier et al. (J. Mol. Biol. (1996) 263: 551-567).

Also encompassed by the invention is a portion of a variable domain that comprises at least one CDR region substantially as set out herein and, optionally, intervening framework regions from the V_{H} or V_{L} domains as set out herein. Variable domains lacking a portion of the N-terminus of the FR1 and/or a portion of the C, terminus of the FR4 are also encompassed by the invention. Additional residues at the N-terminal of the FR1 or C-terminal of the FR4 of the variable domain may not be the same residues found in naturally occurring antibodies. For example, construction of antibodies by recombinant DNA techniques often introduces N- or C-terminal residues from its use of linkers. Some linkers may be used to join variable domains to other variable domains (e.g., diabodies), constant domains, or proteinaceous labels.

Although the embodiments specifically exemplified herein comprise a “matching” pair of V_{H} and V_{L} domains, a skilled artisan will recognize that alternative embodiments may comprise binding proteins containing only a single CDR from either V_{L} or V_{H} domain. Either one of the V_{H} domain or V_{L} domain can be used to screen for complementary domains capable of forming a two-domain specific binding protein capable of, binding to ErbB2 ECD. The screening may be accomplished by phage display screening methods using the so-called hierarchical dual combinatorial approach disclosed in WO 92/01047. In this approach, an individual colony containing either a H or L chain clone is used to infect a complete library of clones encoding the other chain (L or H), and the resulting two-chain specific antigen-binding domain is selected in accordance with phage display techniques as described.

In some alternative embodiments, the anti-ErbB2 binding protein can be linked to a protein (e.g., albumin) by chemical cross-linking or recombinant methods. The disclosed antibodies may also be linked to a variety of nonproteinaceous polymers (e.g., polyethylene glycol, polypropylene glycol, or polyoxyalkylenes) in manners set forth in U.S. Pat. Nos. 4,640,835; 4,496,689; 4,301,144; 4,670,417; 4,791,192; or 4,179,337. The binding proteins can be chemically modified by covalent conjugation to a polymer, for example, to increase their half-life in blood circulation. Exemplary polymers and attachment methods are shown in U.S. Pat. Nos. 4,766,106; 4,179,337; 4,495,285; and 4,609,546.

Binding proteins of the invention can be modified to alter their glycosylation; that is, at least one carbohydrate moiety can be deleted or added to the binding protein, for example to modify antibody dependent (or Fc dependent) cellular cytotoxicity (ADCC/FcDCC), in particular to enhance ADCC/FcDCC.

Deletion or addition of glycosylation sites can be accomplished by changing amino acid sequence to delete or create glycosylation consensus sites, that are well known in the art. Another means of adding carbohydrate moieties is the chemical or enzymatic coupling of glycosides to amino acid residues of the antibody (see WO 87/05330 and Aplin et al. (1981) CRC Crit. Rev. Biochem., 22: 259-306). Removal of carbohydrate moieties can also be accomplished chemically or enzymatically (see Hakimuddin et al. (1987) Arch. Biochem. Biophys., 259: 52; Edge et al. (1981) Anal. Biochem., 118: 131; Thotakura et al. (1987) Meth. Enzymol., 138: 350).

Methods for altering an antibody constant region are known in the art. Antibodies with altered function (e.g., altered affinity for an effector ligand such as FcR on a cell or the C1 component of complement) can be produced by replacing at least one amino acid residue in the constant portion of the antibody with a different residue (see e.g., EP 388,151 A1, U.S. Pat. No. 5,624,821 and U.S. Pat. No. 5,648,260). Similar types of alterations could be described that if applied to a murine or other species antibody would reduce or eliminate similar functions.

For example, it is possible to alter the affinity of an Fc region of an antibody (e.g., an IgG, such as a human IgG) for FcR (e.g., Fc gamma R1) or C1q. The affinity may be altered by replacing at least one specified residue with at least one residue having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

For example, replacing residue 297 (asparagine) with alanine in the IgG constant region significantly inhibits recruitment of effector cells, while only slightly reducing (about three fold weaker) affinity for C1q (see e.g., U.S. Pat. No. 5,624,821). The numbering of the residues in the heavy chain is that of the EU index (see Kabat et al., 1991 supra). This alteration destroys the glycosylation site and it is believed that the presence of carbohydrate is required for Fc receptor binding. Any other substitution at this site that destroys the glycosylation site is believed to cause a similar decrease in lytic activity. Other amino acid substitutions, e.g., changing any one of residues 318 (Glu), 320 (Lys) and 322 (Lys), to Ala, are also known to abolish Clq binding to the Fc region of IgG antibodies (see e.g., U.S. Pat. No. 5,624,821).

Modified binding proteins can be produced that have a reduced interaction with an Fc receptor. For example, it has been shown that in human IgG₃, which binds to the human Fc gamma R1 receptor, changing Leu 235 to Glu destroys its interaction with the receptor. Mutations on adjacent or close sites in the hinge link region of an antibody (e.g., replacing residues 234, 236 or 237 with Ala) can also be used to affect antibody affinity for the Fc gamma R1 receptor. The numbering of the residues in the heavy chain is based in the EU index (see Kabat et al., 1991 supra).

Additional methods for altering the lytic activity of an binding protein, for example, by altering at least one amino acid in the N-terminal region of the C_{H}2 domain, are described in WO 94/29351 by Morgan et al. and U.S. Pat. No. 5,624,821.

One of skill in the art will appreciate that the modifications described above are not all-exhaustive, and that many other modifications are obvious to a skilled artisan in light of the teachings of the present disclosure.

A binding protein of this invention may be tagged with a detectable or functional label. These labels include radiolabels (e.g., ¹³¹I or ⁹⁹Tc), enzymatic labels (e.g., horseradish peroxidase or alkaline phosphatase), and other chemical moieties (e.g., biotin).

In some embodiments, the invention features a human, monoclonal antibody that specifically binds the ECD, ErbB2, in particular, human ErbB2 and posseses one or more of the following characteristics: (1) it is an in vitro generated antibody (2) it is an in vivo generated antibody (e.g., transgenic mouse system); (3) it binds to ErbB2 with an association constant of at least 10¹² M⁻¹; (4) it binds to ErbB2 with an association constant of at least 10¹¹ M⁻¹; (5) it binds to ErbB2 with an association constant of at least 10¹⁰ M⁻¹; (6) it binds to ErbB2 with an association constant of at least 10⁹ M⁻¹; (7) it binds to ErbB2 with an association constant of at least 10⁶ M⁻¹; (8) it binds to ErbB2 with a dissociation constant of 500 nM or less; (9) it binds to ErbB2 with a dissociation constant of 10 nM or less; (10) it binds to ErbB2 with a dissociation constant of 150 pM or less; (11) it binds to ErbB2 with a dissociation constant of 60 pM or less.

### III. Nucleic Acids, Cloning and Expression Systems

In another aspect, the invention provides isolated nucleic acids encoding an anti-ErbB2 binding protein of the invention. The nucleic acids may comprise DNA or RNA, and they may be synthetic (completely or partially) or recombinant (completely or partially). Reference to a nucleotide sequence as set out herein encompasses a DNA molecule with the specified sequence, and encompasses a RNA molecule with the specified sequence in which U is substituted for T.

The invention also contemplates nucleic acids that comprise a coding sequence for a CDR1, CDR2 or CDR3, a frame-work sequence (including FR1, FR2, FR3 and/or FR4), a V_{H} domain, a V_{L} domain, or combinations thereof, as disclosed herein, or a sequence substantially identical thereto (e.g., a sequence at least 85%, 90%, 95%, 96%, 97%, 98%, 99% or higher identical thereto, or that is capable of hybridizing under stringent conditions to the sequences disclosed).

In one embodiment, the isolated nucleic acid has a nucleotide sequence encoding a heavy chain variable region and/or a light chain variable region of an anti-ErbB2 binding protein comprising at least one heavy chain CDR or light chain CDR, respectively, chosen from the CDR amino acid sequences found in SEQ ID Nos:1-95, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 507, 509, 511, 513, 515, 517, 519, 521, 523, 525, or a sequence encoding a CDR that differs by one or two amino acids from the CDR sequences set forth herein. In some embodiments, the nucleic acid encodes an anti-ErbB2 binding protein comprising one, two, or all 3 heavy chain CDRs, one, two or all 3 light chain CDRs or all 6 CDRS in any of an specifically exemplified antibody.

The nucleic acid can encode only the light chain or the heavy chain variable region, or can also encode an antibody light or heavy chain constant region, operatively linked to the corresponding variable region. In one embodiment, the light chain variable region is linked to a constant region chosen from a kappa or a lambda constant region. The light chain constant region may also be a human kappa or lambda type. In another embodiment, the heavy chain variable region is linked to a heavy chain constant region of an antibody isotype chosen from IgG (e.g., IgG₁, IgG₂, IgG₃, IgG₄), IgM, IgA₁, IgA₂, IgD, and IgE. The heavy chain constant region may be an IgG (e.g., an IgG₁) isotype.

The nucleic acid compositions of the present invention, while often in the native sequence (of cDNA or genomic DNA or mixtures thereof) except for modified restriction sites and the like, may be mutated in accordance with standard techniques to provide gene sequences. For coding sequences, these mutations, may affect amino acid sequence as desired. In particular, nucleotide sequences substantially identical to or derived from native V, D, J, constant, switches and other such sequences described herein are contemplated (where “derived” indicates that a sequence is identical or modified from another sequence).

In one embodiment, the nucleic acid differs (e.g., differs by substitution, insertion, or deletion) from that of the sequences provided (e.g., as follows: by at least one but less than 10, 20, 30, or 40 nucleotides; at least one but less than 1%, 5%, 10% or 20% of the nucleotides in the subject nucleic acid). Also within the invention are ErbB2 binding proteins encoded by a nucleic acid that hybridizes under stringent conditions to a nucleic acid specifically exemplified herein or to its complement. If necessary for this analysis the sequences should be aligned for maximum homology. “Looped out” sequences from deletions or insertions, or mismatches, are considered differences. The difference may be at a nucleotide(s) encoding a non-essential residue(s), or the difference may be a conservative substitution(s).

The invention also provides nucleic acid constructs in the form of plasmids, vectors, transcription or expression cassettes, that comprise at least one nucleic acid as described herein as well as a host cell that comprises at least one nucleic acid described herein. Suitable host cells for the expression of a binding protein of the invention well be well known in the art and include mammalian, plant, insects, bacterial or yeast cells.

Also provided are the methods of making an anti-ErbB2 antibody of the invention that is encoded by the nucleic acid(s) comprising sequence described herein. The method comprises culturing host cells under appropriate conditions to express the protein from the nucleic acid. Following expression and production, the encoded pp may be isolated and/or purified using any suitable technique, then used as appropriate. The method can also include the steps of fusing a nucleic acid encoding a scFv with nucleic acids encoding a Fc portion of an antibody and expressing the fused nucleic acid in a cell. The method can also include a step of germlining.

Antigen-binding fragments, V_{H} and/or V_{L} domains, and encoding nucleic acid molecules and vectors may be isolated and/or purified from their natural environment, in substantially pure or homogenous form, or, in the case of nucleic acid, free or substantially free of nucleic acid or genes of origin other than the sequence encoding a polypeptide with the require function.

Systems for cloning and expressing polypeptides in a variety of host cells are known in the art. Cells suitable for producing antibodies are described in, for example, Fernandez et al. (1999) Gene Expression Systems, Academic Press, eds. In brief, suitable host cells include mammalian cells, insect cells, plant cells, yeast cells, or prokaryotic cells, e.g., E. coli. Mammalian cells available in the art for heterologous polypeptide expression include lymphocytic cell lines (e.g., NSD), HEK293 cells, Chinese hamster ovary (CHO) cells, COS cells, HeLa cells, baby hamster kidney cells, oocyte cells, and cells from a transgenic animal, e.g., mammary epithelial cell.

In one embodiment, all or a portion of an anti-ErbB2 antibody selected from S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H11, S6R3_CS_{—}1G5, S6R2_DP47_{—}1H11, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9 is expressed in HEK293 or CHO cells. In other embodiments, one or more nucleic acids encoding an anti-ErbB2 binding protein of the invention are placed under the control of a tissue-specific promoter (e.g., a mammary specific promoter) and the antibodies are produced in transgenic animals. For example, the antibodies are secreted into the milk of the transgenic animal, such as a transgenic cow, pig, horse, sheep, goat or rodent.

Suitable vectors may be chosen or constructed to contain appropriate regulatory sequences, including promoter sequences, terminator sequences, polyadenylation sequences, enhancer sequences, marker genes, and other sequences. The vectors may also contain a plasmid or viral backbone. For details, see Sambrook et al., Molecular Cloning: A Laboratory Manual, 2nd ed., Cold Spring Harbor Laboratory Press (1989). Many established techniques used with vectors, including the manipulation, preparation, mutagenesis, sequencing, and transfection of DNA, are described in Current Protocols in Molecular Biology, Second Edition, Ausubel et al. eds., John Wiley & Sons (1992).

A nucleic acid encoding all or]part of an anti-ErbB2 binding protein of the invention may be introduced into a host cell by any readily available means. For eukaryotic cells, suitable transfection techniques may include calcium phosphate, DEAE-Dextran, electroporation, liposome-mediated transfection, and transduction using retrovirus or other viruses, e.g., vaccinia or baculovirus. For bacterial cells, suitable techniques may include calcium chloride transformation, electroporation, and transfection using bacteriophage. DNA introduction may be followed by a selection method (e.g., drug resistance) to select cells that contain the nucleic acid.

### IV. Therapeutic Uses of Anti-ErbB2 Binding Proteins

Anti-ErbB2 binding proteins of the invention may be ErbB2 agonists or antagonists. An agonist ErbB2 binder of the invention increases HER2 tyrosine phosphorylation in the absence or presence of other HER2 agonists such as Heregulin or Epidermal Growth Factor (EGF). Certain HER2 agonists of the invention increase phosphorylation of HER2 pathway proteins. In some embodiments, the agonist of the invention increase phosphorylation of AKT, MAPK and/or ERK. In some embodiments, the HER2 agonist of the invention decreases proliferation and/or increases cell death of a cancer cell, in vitro and in vivo.

Anti-ErbB2 binding proteins that act as antagonists to ErbB2 can be used to reduce at least one ErbB2-mediated activity, such as reducing ErbB2-mediated tyrosine phosphorylation, decreased heterodimerization of ErbB2 with other ERBB-family members, decreased ErbB2-mediated cell signalling and decreased growth or proliferation of ErbB2-expressing cells. In one embodiment, anti-ErbB2 binding proteins of the invention are used in a method for decreasing tumor growth, the method comprising contacting an ErbB2 expressing cell with a binding protein of the invention to modulate cell proliferation, cytolytic activity, cytokine secretion, or chemokine secretion.

Accordingly, the binding proteins of the invention can be used to directly or indirectly inhibit or reduce the activity (e.g., proliferation, differentiation, and/or survival) of cells expressing ErbB2, and, thus, can be used to treat a variety of disorders including hyperproliferative disorders.

The binding proteins of the invention can be used to treat hyperproliferative disorders associated with activity of ErbB2 by administering the antibodies in an amount sufficient to inhibit or reduce hyperproliferation and/or to increase cell death, such as by apoplosis of ErbB2 expressing cells in a subject and allowing the antibodies to treat or prevent the disorder. ErbB2 is expressed in a number of cancers including, but not limited to, breast, bladder, cervical, ovarian, prostate, testicular, oral, colorectal, lung and pancreatic, cancers and in childhood medulloblastoma, oral squamous cell carcinoma, gastric cancer cholangio carcinoma, osteosarcoma, primary Fallopian tube carcinoma, salivary gland tumors and synovial sarcoma. Binding proteins of the invention may be used to inhibit the progression of neoplasms, e.g. squamous cell carcinomas, basal cell carcinomas, transitional cell papillomas and carcinomas, adenomas, adenocarcinoma. According to the invention, an anti-ErbB2 binding protein of the invention can be administered to a subject in need thereof as part of a regimen that comprises another therapeutic modality, such as surgery or radiation.

### V. Combination Therapy

According to the invention, a composition suitable for pharmaceutical use comprising at least one anti-ErbB2 binding protein further comprises at least one additional therapeutic agent. The therapy is useful for treating ErbB2-mediated pathological conditions or disorders including cancer. The term “in combination” in this context means that the binding protein composition and the additional therapeutic agent are given as part of a treatment regimen. In some embodiments, the anti-ErbB2 binding protein is administered substantially contemporaneously, either simultaneously or sequentially with another therapeutic agent, including one being a pretreatment in relation to the other. In some embodiments, in which administration is sequential, at the onset of administration of the second agent, the first of the two agents is still detectable at effective concentrations at the site of treatment. In another embodiment, if given sequentially, at the onset of administration of the second compound, the first of the two compounds is not detectable at effective concentrations at the site of treatment.

According to the invention, a treatment regimen may comprise two or more anti-ErbB2 antibodies of the invention. The binding molecules may be ones that bind the same or nearby regions of HER2, as illustrated for example by blocking or cross-blocking each other's binding to HER2, or they may bind to different regions of HER2, as shown by lack of cross-blocking. Two or more anti-ErbB2 binding molecules of the invention may be co-formulated, co-administered or merely be part of the same treatment regimen.

For example, the combination therapy can include at least one anti-ErbB2 binding protein of the invention co-formulated with, co-administered with, or administered as part of the same therapeutic regimen as at least one additional therapeutic agent. The additional agents may include at least but is not limited to mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, antiproliferative agents, kinase inhibitors, angiogenesis inhibitors, growth factor inhibitors, cox-I inhibitors, cox-II inhibitors, radiation, cell cycle inhibitors, enzymes, anti-hormones, statins, and anti-androgens.

In other embodiments, at least one anti-ErbB2 binding protein can be co-formulated with, and/or co-administered with, at least one anti-inflammatory drug, immunosuppressant, metabolic inhibitor, and enzymatic inhibitor.

In other embodiments, an anti-ErbB2 antibody can be used in combination with at least one binding protein, such as an antibody, directed at other cancer targets. Another aspect of the present invention accordingly relates to kits for carrying out the administration of the anti-ErbB2 binding protein alone or in combination with other therapeutic agents. In one embodiment, the kit comprises at least one anti-ErbB2 binding protein formulated in a pharmaceutical carrier, and at least one additional therapeutic agent, formulated as appropriate in one or more separate pharmaceutical preparations.

In one embodiment, the present inventive binding proteins can be administered in combination with (e.g., prior to, concurrently with, or subsequent to) one or more other therapeutic agents. Such therapeutic agents include, for example, cytotoxic agents that inhibit or prevent the function of cells and/or causes destruction of cells. The term is intended to include radioactive isotopes (e.g. I131, I125, Y90 and Re186), chemotherapeutic agents, growth inhibitory agents, cytokine, and toxins such as enzymatically active toxins of bacterial, fungal, plant or animal origin, or fragments thereof.

Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A growth inhibitory agent when used herein refers to a compound or composition that inhibits growth of a cell, especially an ErbB2-overexpressing cancer cell either in vitro or in vivo. In the context of the present invention, the growth inhibitory agent can be one that significantly reduces the percentage of ErbB2 overexpressing cells in S phase and the binding proteins of the present invention may potentially sensitize the cells to such an S phase agent. S-phase blockers include the vincas (vincristine and vinblastine), taxol, and topo II inhibitors such as doxorubicin, daunorubicin, etoposide, and bleomycin. Examples of growth inhibitory agents include agents that block cell cycle progression (at a place other than S phase), include agents that induce G1 arrest and M-phase arrest. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995), especially p. 13.

Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor, fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The invention also pertains to immunoconjugates comprising the binding proteins described herein conjugated to a cytotoxic agent such as a chemotherapeutic agent, toxin (e.g. an enzymatically active toxin of bacterial, fungal, plant or animal origin, or fragments thereof), or a radioactive isotope (i.e., a radioconjugate). Such immunconjugates are particularly indicated for those binding proteins of the invention that internalize in Her2 expressing cells, as shown in the Examples section.

Chemotherapeutic agents useful in the generation of such immunoconjugates have been described above. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are available for the production of radioconjugated anti-ErbB2 binding proteins. Examples include 212Bi, 131I, 131In, 90 Y and 186Re.

Immunoconjugates comprising a member of the potent family of antibacterial and antitumor agents, known collectively as the calicheamicins or the LL-E33288 complex, (see U.S. Pat. No. 4,970,198 (1990)) are also contemplated. The most potent of the calicheamicins is designated γ 1, which is herein referenced simply as gamma. These compounds contain a methyltrisulfide that can be reacted with appropriate thiols to form disulfides, at the same time introducing a functional group such as a hydrazide or other functional group that is useful in attaching a calicheamicin derivative to a carrier. (See U.S. Pat. No. 5,053,394). Conjugation methods for preparing monomeric calicheamicin derivative/carrier have been disclosed (see U.S. Pat. No. 5,712,374 and U.S. Pat. No. 5,714,586, incorporated herein in their entirety).

Conjugates of the binding protein and cytotoxic agent can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the binding protein.

Effective amounts of the other therapeutic agents are well known to those skilled in the art. However, it is well within the skilled artisan's purview to determine the other therapeutic agent's optimal effective amount range. The binding proteins of the present invention and the other therapeutic agent(s) can act additively or, alternatively, synergistically. In one embodiment of the invention, where another therapeutic agent(s) is administered to an animal, either the effective amount of the binding protein of the present invention or the other therapeutic agent(s) can be administered in an amount that is less than its effective amount would be where the other therapeutic agent is not administered. In this case, without being bound by theory, it is believed that the two (or more) act synergistically.

### VI. Diagnostic Uses

In a further aspect, a binding protein of the invention may also be used to detect the presence of ErbB2 or ErbB2 expressing cells in a biological sample. By correlating the presence or level of ErbB2 with a medical condition, one of skill in the art can diagnose the associated medical condition, including cancer.

Binding protein-based, including antibody-based detection methods are well known in the art, and include ELISA, radioimmunoassays, immunoblots, Western blots, flow cytometry, immunofluorescence, immunoprecipitation, and other related techniques. The antibodies may be provided in a diagnostic kit that incorporates at least one of these procedures to detect ErbB2. The kit may contain other components, packaging, instructions, or other material to aid the detection of the protein and use of the kit.

Binding proteins of the invention may be modified with detectable markers, including ligand groups (e.g., biotin), fluorophores and chromophores, radioisotopes, electron-dense reagents, or enzymes. Enzymes are detected by their activity. For example, horseradish peroxidase is detected by its ability to convert tetramethylbenzidine (TMB) to a blue pigment, quantifiable with a spectrophotometer. Other suitable binding partners include biotin and avidin, IgG and protein A, and other receptor-ligand pairs known in the art.

Binding proteins of the invention can also be functionally linked (e.g., by chemical coupling, genetic fusion, non-covalent association or otherwise) to at least one other molecular entity, such as another antibody (e.g., a bispecific or a multispecific antibody), toxins, radioisotopes, cytotoxic or cytostatic agents, among others for therapeutic use. Other permutations and possibilities are apparent to those of ordinary skill in the art, and they are considered equivalents within the scope of this invention.

Further, the anti-ERRB2 binding proteins can be used to detect the presence, isolate, and/or to quantitate ErbB2-expressing cells in a sample from a subject or by in vivo imaging.

### VII. Pharmaceutical Compositions and Methods of Administration

In still another aspect, the invention provides compositions comprising an anti-ErbB2 binding protein of the invention. The compositions may be suitable for pharmaceutical use and administration to patients. The compositions comprise a binding protein of the present invention and a pharmaceutically acceptable carrier. The composition may optionally comprise a pharmaceutical excipient. As used herein, “pharmaceutical excipient” includes solvents, dispersion media, coatings, antibacterial and antifungal agents, isotonic and absorption delaying agents, etc., that are compatible with pharmaceutical administration. Use of these agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. Pharmaceutical compositions may be topically or orally administered, or capable of transmission across mucous membranes. Examples of administration of a pharmaceutical composition include oral ingestion or inhalation. Administration may also be intravenous, intraperitoneal, intramuscular, intracavity, subcutaneous, cutaneous, or transdermal.

Solutions or suspensions used for intradermal or subcutaneous application typically include at least one of the following components: a sterile diluent such as water, saline solution, fixed oils, polyethylene glycol, glycerine, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

Solutions or suspensions used for intravenous administration include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent that delays absorption, e.g., aluminum monostearate and gelatin.

Oral compositions include an inert diluent or edible carrier. The composition can be enclosed in gelatin or compressed into tablets. For the purpose of oral administration, the antibodies can be incorporated with excipients and placed in tablets, troches, or capsules. Pharmaceutically compatible binding agents or adjuvant materials can be included in the composition. The tablets, troches, and capsules, may contain (1) a binder such as microcrystalline cellulose, gum tragacanth or gelatin; (2) an excipient such as starch or lactose, (3) a disintegrating agent such as alginic acid, Primogel, or corn starch; (4) a lubricant such as magnesium stearate; (5) a glidant such as colloidal silicon dioxide; or (6) a sweetening agent or a flavoring agent.

The composition may also be administered by a transmucosal or transdermal route. For example, antibodies that comprise a Fc portion may be capable of crossing mucous membranes in the intestine, mouth, or lungs (via Fc receptors). Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the antibodies are delivered in an aerosol spray from a pressured container or dispenser, that contains a propellant (e.g., liquid or gas) or a nebulizer.

In certain embodiments, the binding proteins of this invention are prepared with carriers to protect against rapid elimination from the body. Biodegradable polymers (e.g., ethylene vinyl acetate, polyanhydrides, polyglycolic acid, collagen, polyorthoesters, polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811).

The binding proteins or compositions of the invention are administered in therapeutically effective amounts as described. Therapeutically effective amounts may vary with the subject's age, condition, sex, and severity of medical condition. Appropriate dosage may be determined by a physician based on clinical indications. The binding proteins or compositions may be given as a bolus dose to maximize the circulating levels of protein for the greatest length of time. Continuous infusion may also be used after the bolus dose.

As used herein, the term “subject” is intended to include human and non-human animals. Subjects may include a human patient having a disorder characterized by cells that express ErbB2, e.g., a cancer cell or an immune cell. The term “non-human animals” of the invention includes all vertebrates, such as non-human primates, sheep, dogs, cows, chickens, amphibians, reptiles, etc.

Examples of dosage ranges that can be administered to a subject can be chosen from: 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg/kg to 1 mg/kg, 250 μg/kg to 2 mg/kg, 250 μg/kg to 1 mg/kg, 500 μg/kg to 2 mg/kg, 500 μg/kg to 1 mg/kg, 1 mg/kg to 2 mg/kg, 1 mg/kg to 5 mg/kg, 5 mg/kg to 10 mg/kg, 10 mg/kg to 20 mg/kg, 15 mg/kg to 20 mg/kg, 10 mg/kg to 25 mg/kg, 15 mg/kg to 25 mg/kg, 20 mg/kg to 25 mg/kg, and 20 mg/kg to 30 mg/kg (or higher). These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration.

In certain circumstances it may be advantageous to formulate compositions in dosage unit form for ease of administration and uniformity of dosage. Dosage unit form as used herein refers to physically discrete units suited for the patient. Each dosage unit contains a predetermined quantity of antibody calculated to produce a therapeutic effect in association with the carrier. The dosage unit depends on the characteristics of the antibodies and the particular therapeutic effect to be achieved.

Toxicity and therapeutic efficacy of the composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Binding proteins that exhibit large therapeutic indices may be less toxic and/or more therapeutically effective.

The data obtained from the cell culture assays and animal studies can be used to formulate a dosage range in humans. The dosage of these compounds may lie within the range of circulating antibody concentrations in the blood, that includes an ED₅₀ with little or no toxicity. The dosage may vary within this range depending upon the dosage composition form employed and the route of administration. For any antibody used in the present invention, the therapeutically effective dose can be estimated initially using cell culture assays. A dose may be formulated in animal models to achieve a circulating plasma concentration range that includes the IC₅₀ (i.e., the concentration of antibody that achieves a half-maximal inhibition of symptoms). The effects of any particular dosage can be monitored by a suitable bioassay. Examples of suitable bioassays include DNA replication assays, transcription-based assays and ErbB2 binding assays.

### EXAMPLES

### Example 1

### Selection of Anti-ErbB2 scFv's

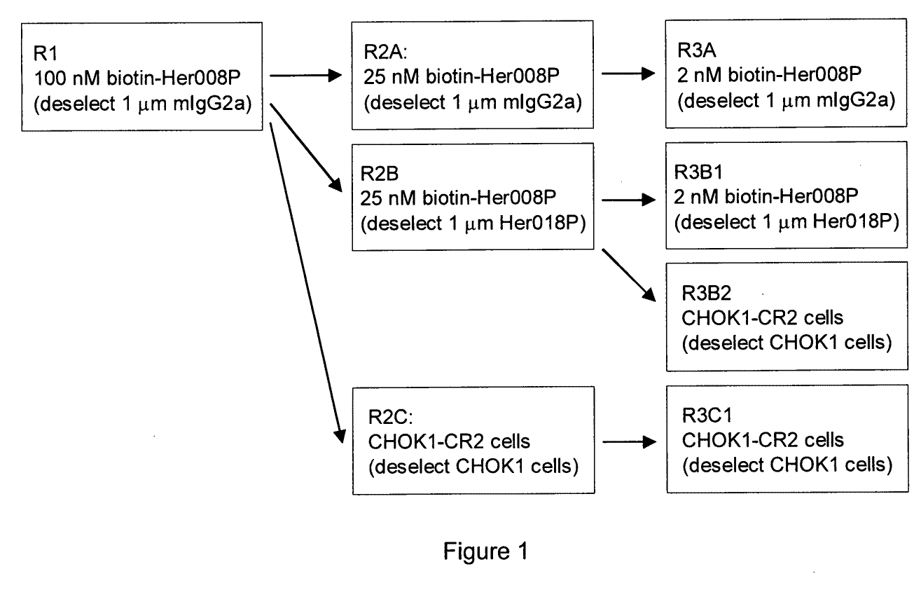

Single chain fragment variable (scFv) moieties that bind to the extracellular domain (ECD) of Her2 (ErbB2) were identified following three rounds of selection using three phagemid libraries: the Bone Marrow Vaughan (BMV) library (Vaughan et al, 1996), the combined spleen (CS) library and the DP47 library (unpublished). Several Her2-Fc proteins or cell lines expressing various forms of Her2 were used during the selection and subsequent screening steps (see Table 3). The selection strategies are outlined in FIG. 1.

Selection Using Biotinylated HER2 Proteins

For selections involving biotinylated protein, aliquots of phage and magnetic streptavidin beads (Dynabeads M-280 streptavidin) were blocked separately in 3% milk/PBS for 1 hour at room temperature in a rotary mixer (20 rpm). Each selection was preceded by a de-selection step. For de-selection, blocked phage were incubated with the pre-blocked magnetic beads and incubated for one hour on a rotary shaker (20 rpm). The de-selected library was collected by pelleting the beads using a magnetic separator. A 1 μM concentration of a non-biotinylated competitor protein (eg, irrelevant MlgG2a protein) was added to the de-selected phage and incubated for a further hour.

Biotinylated selection antigen (at various concentrations as indicated in FIG. 1) was incubated with the de-selected phage library for 2 hours at room temp on a rotary mixer (20 rpm) followed by a 15 minute incubation with pre-blocked magnetic beads. Beads were separated using a magnetic separator and washed 10 times with PBS/0.1% Tween 20 and 3 times with PBS. Bound phage were eluted by incubation with a 10 ug/ml solution of trypsin in PBS for 30 minutes at 37° C. (100 rpm) followed by separation from the magnetic beads.

Selection Using Cells Expressing HER2ECD or ECD Fragments

For selections involving cells, approximately 4×10⁷ de-selection cells (ie. cells not expressing the antigen of interest) and 2×10⁷ capture (i.e., selection) cells (cells expressing the antigen of interest) were collected using PBS/5 mM EDTA and washed twice with PBS. Cells were blocked with 3% milk/1% BSA/PBS for 1 hour at 4° C. on a rotary mixer (20 rpm). De-selection cells were collected by centrifugation, re-suspended in blocked phage and incubated at 4° C. as before. Both the capture and de-selection cells were pelleted and the capture cells were resuspended with the de-selected phage supernatant and incubated at 4° C. as before. The capture cells were washed three times with cold PBS/0.1% Tween 20 and three times with cold PBS. Phage were eluted by re-suspending the cells in a 10 μg/ml trypsin solution and incubated for 30 min at 37° C. (100 rpm). Eluted phage were harvested in the supernatant following centrifugation of cells. Eluted phage were used to infect 10 ml of an E. coli TG1 culture that had been grown to mid-logarithmic phase (corresponding to an OD₆₀₀ of ˜0.5). Bacteria were infected with phage for 1 hour at 37° C. with shaking at 150 rpm, concentrated following a centrifugation step and plated on 2×TY agar bioassay plates containing 2% glucose and 100 ug/ml ampicillin (2×TYAG). Various dilutions of E. coli culture infected with either input or output phage were also plated on 2×TYAG agar to determine phage titers. Following overnight growth at 30° C., 10 ml of 2×TYAG medium was added to each bioassay plate and the cells were re-suspended by scraping the bacterial lawn. Glycerol was added to this cell suspension to give a final concentration of 17% and stored in aliquots at −80° C. until further use. To rescue phage for the next round of selection, 100 μl of this cell suspension was used to inoculate 20 ml 2×TYAG medium, that was grown at 37° C. (300 rpm) to an OD₆₀₀ of 0.3-0.5. Cells were then super-infected with 3.3 μl of MK13K07 helper phage and incubated at 37° C. (150 rpm) for 1 hour. The cells were then centrifuged and the pellet re-suspended in a kanamycin/non-glucose containing medium (2×TY with 50 μg/ml kanamycin and 100 ug/ml ampicillin). This culture was grown overnight at 30° C. (300 rpm). Phage were harvested in the supernatant following centrifugation and were ready to use in the second and third rounds of selection as described in FIG. 1.

TABLE 3

| | | Sequence for Her2 region of fusion |
|---|---|---|
| Name | Description | protein |
| Her008P | Full-length extracellular | MELAALCRWGLLLALLPPGAASTQVCT |
| (Synonyms: | domain (ECD) of Her2 | GTDMKLRLPASPETHLDMLRHLYQGC |
| ECD; SIIS; | expressed with a mIgG2a Fc | QVVQGNLELTYLPTNASLSFLQDIQEV |
| HER008) | tail | QGYVLIAHNQVRQVPLQRLRIVRGTQL |
| | | FEDNYALAVLDNGDPLNNTTPVTGASP |
| | | GGLRELQLRSLTEILKGGVLIQRNPQLC |
| | | YQDTILWKDIFHKNNQLALTLIDTNRSR |
| | | ACHPCSPMCKGSRCWGESSEDCQSL |
| | | TRTVCAGGCARCKGPLPTDCCHEQCA |
| | | AGCTGPKHSDCLACLHFNHSGICELHC |
| | | PALVTYNTDTFESMPNPEGRYTFGASC |
| | | VTACPYNYLSTDVGSCTLVCPLHNQEV |
| | | TAEDGTQRCEKCSKPCARVCYGLGME |
| | | HLREVRAVTSANIQEFAGCKKIFGSLAF |
| | | LPESFDGDPASNTAPLQPEQLQVFETL |
| | | EEITGYLYISAWPDSLPDLSVFQNLQVI |
| | | RGRILHNGAYSLTLQGLGISWLGLRSL |
| | | RELGSGLALIHHNTHLCFVHTVPWDQL |
| | | FRNPHQALLHTANRPEDECVGEGLAC |
| | | HQLCARGHCWGPGPTQCVNCSQFLR |
| | | GQECVEECRVLQGLPREYVNARHCLP |
| | | CHPECQPQNGSVTCFGPEADQCVACA |
| | | HYKDPPFCVARCPSGVKPDLSYMPIW |
| | | KFPDEEGACQPCPINCTHSCVDLDDKG |
| | | CPAEQRASPLTSIIS (SEQ ID NO: 242) |
| Her017P | Her2 ECD with a deletion in | MELAALCRWGLLLALLPPGAASTQVCT |
| (Synonyms: | the membrane proximal 9 | GTDMKLRLPASPETHLDMLRHLYQGC |
| EQR; | amino acids expressed with | QVVQGNLELTYLPTNASLSFLQDIQEV |
| HER017) | a mIgG2a Fc tail | QGYVLIAHNQVRQVPLQRLRIVRGTQL |
| | | FEDNYALAVLDNGDPLNNTTPVTGASP |
| | | GGLRELQLRSLTEILKGGVLIQRNPQLC |
| | | YQDTILWKDIFHKNNQLALTLIDTNRSR |
| | | ACHPCSPMCKGSRCWGESSEDCQSL |
| | | TRTVCAGGCARCKGPLPTDCCHEQCA |
| | | AGCTGPKHSDCLACLHFNHSGICELHC |
| | | PALVTYNTDTFESMPNPEGRYTFGASC |
| | | VTACPYNYLSTDVGSCTLVCPLHNQEV |
| | | TAEDGTQRCEKCSKPCARVCYGLGME |
| | | HLREVRAVTSANIQEFAGCKKIFGSLAF |
| | | LPESFDGDPASNTAPLQPEQLQVFETL |
| | | EEITGYLYISAWPDSLPDLSVFQNLQVI |
| | | RGRILHNGAYSLTLQGLGISWLGLRSL |
| | | RELGSGLALIHHNTHLCFVHTVPWDQL |
| | | FRNPHQALLHTANRPEDECVGEGLAC |
| | | HQLCARGHCWGPGPTQCVNCSQFLR |
| | | GQECVEECRVLQGLPREYVNARHCLP |
| | | CHPECQPQNGSVTCFGPEADQCVACA |
| | | HYKDPPFCVARCPSGVKPDLSYMPIW |
| | | KFPDEEGACQPCPINCTHSCVDLDDKG |
| | | CPAEQR (SEQ ID NO: 243) |
| Her018P | Her2 ECD with a deletion in | MELAALCRWGLLLALLPPGAASTQVCT |
| (Synonyms: | the CR2 (Domain IV) region | GTDMKLRLPASPETHLDMLRHLYQGC |
| 1.8; | expressed with a mIgG2a Fc | QVVQGNLELTYLPTNASLSFLQDIQEV |
| HER018) | tail | QGYVLIAHNQVRQVPLQRLRIVRGTQL |
| | | FEDNYALAVLDNGDPLNNTTPVTGASP |
| | | GGLRELQLRSLTEILKGGVLIQRNPQLC |
| | | YQDTILWKDIFHKNNQLALTLIDTNRSR |
| | | ACHPCSPMCKGSRCWGESSEDCQSL |
| | | TRTVCAGGCARCKGPLPTDCCHEQCA |
| | | AGCTGPKHSDCLACLHFNHSGICELHC |
| | | PALVTYNTDTFESMPNPEGRYTFGASC |
| | | VTACPYNYLSTDVGSCTLVCPLHNQEV |
| | | TAEDGTQRCEKCSKPCARVCYGLGME |
| | | HLREVRAVTSANIQEFAGCKKIFGSLAF |
| | | LPESFDGDPASNTAPLQPEQLQVFETL |
| | | EEITGYLYISAWPDSLPDLSVFQNLQVI |
| | | RGRILHNGAYSLTLQGLGISWLGLRSL |
| | | RELGSGLALIHHNTHLCFVHTVPWDQL |
| | | FRNPHQALLHTANRPEDECVGEGLAC |
| | | HQLCARGHCWGPGPTQCVNCSQFLR |
| | | GQECVEECRVLQGLPREYVNARHCLP |
| | | CHPECQPQNGSVTCFGPEADQCVACA |
| | | HYKDPPFCVAR (SEQ ID NO: 244) |
| Her054P | Domains I (L1) and II (CR-1) | MELAALCRWGLLLALLPPGAASTQVCT |
| (Synonyms: | of Her2 expressed with a | GTDMKLRLPASPETHLDMLRHLYQGC |
| L1-CR1; | mIgG2a Fc tail | QVVQGNLELTYLPTNASLSFLQDIQEV |
| 1.0) | | QGYVLIAHNQVRQVPLQRLRIVRGTQL |
| | | FEDNYALAVLDNGDPLNNTTPVTGASP |
| | | GGLRELQLRSLTEILKGGVLIQRNPQLC |
| | | YQDTILWKDIFHKNNQLALTLIDTNRSR |
| | | ACHPCSPMCKGSRCWGESSEDCQSL |
| | | TRTVCAGGCARCKGPLPTDCCHEQCA |
| | | AGCTGPKHSDCLACLHFNHSGICELHC |
| | | PALVTYNTDTFESMPNPEGRYTFGASC |
| | | VTACPYNYLSTDVGSCTLVCPLHNQEV |
| | | TAEDGTQRCEKCSKPC (SEQ ID NO: 245) |
| Full length | | MELAALCRWGLLLALLPPGAASTQVCT |
| HER2 | | GTDMKLRLPASPETHLDMLRHLYQGC |
| | | QVVQGNLELTYLPTNASLSFLQDIQEV |
| | | QGYVLIAHNQVRQVPLQRLRIVRGTQL |
| | | FEDNYALAVLDNGDPLNNTTPVTGASP |
| | | GGLRELQLRSLTEILKGGVLIQRNPQLC |
| | | YQDTILWKDIFHKNNQLALTLIDTNRSR |
| | | ACHPCSPMCKGSRCWGESSEDCQSL |
| | | TRTVCAGGCARCKGPLPTDCCHEQCA |
| | | AGCTGPKHSDCLACLHFNHSGICELHC |
| | | PALVTYNTDTFESMPNPEGRYTFGASC |
| | | VTACPYNYLSTDVGSCTLVCPLHNQEV |
| | | TAEDGTQRCEKCSKPCARVCYGLGME |
| | | HLREVRAVTSANIQEFAGCKKIFGSLAF |
| | | LPESFDGDPASNTAPLQPEQLQVFETL |
| | | EEITGYLYISAWPDSLPDLSVFQNLQVI |
| | | RGRILHNGAYSLTLQGLGISWLGLRSL |
| | | RELGSGLALIHHNTHLCFVHTVPWDQL |
| | | FRNPHQALLHTANRPEDECVGEGLAC |
| | | HQLCARGHCWGPGPTQCVNCSQFLR |
| | | GQECVEECRVLQGLPREYVNARHCLP |
| | | CHPECQPQNGSVTCFGPEADQCVACA |
| | | HYKDPPFCVARCPSGVKPDLSYMPIW |
| | | KFPDEEGACQPCPINCTHSCVDLDDKG |
| | | CPAEQRASPLTSIISAVVGILLVVVLGVV |
| | | FGILIKRRQQKIRKYTMRRLLQETELVE |
| | | PLTPSGAMPNQAQMRILKETELRKVKV |
| | | LGSGAFGTVYKGIWIPDGENVKIPVAIK |
| | | VLRENTSPKANKEILDEAYVMAGVGSP |
| | | YVSRLLGICLTSTVQLVTQLMPYGCLLD |
| | | HVRENRGRLGSQDLLNWCMQIAKGMS |
| | | YLEDVRLVHRDLAARNVLVKSPNHVKIT |
| | | DFGLARLLDIDETEYHADGGKVPIKWM |
| | | ALESILRRRFTHQSDVWSYGVTVWEL |
| | | MTFGAKPYDGIPAREIPDLLEKGERLPQ |
| | | PPICTIDVYMIMVKCWMIDSECRPRFRE |
| | | LVSEFSRMARDPQRFVVIQNEDLGPAS |
| | | PLDSTFYRSLLEDDDMGDLVDAEEYLV |
| | | PQQGFFCPDPAPGAGGMVHHRHRSS |
| | | STRSGGGDLTLGLEPSEEEAPRSPLAP |
| | | SEGAGSDVFDGDLGMGAAKGLQSLPT |
| | | HDPSPLQRYSEDPTVPLPSETDGYVAP |
| | | LTCSPQPEYVNQPDVRPQPPSPREGP |
| | | LPAARPAGATLERPKTLSPGKNGVVKD |
| | | VFAFGGAVENPEYLTPQGGAAPQPHP |
| | | PPAFSPAFDNLYYWDQDPPERGAPPS |
| | | TFKGTPTAENPEYLGLDVPV (SEQ ID NO: 246) |

### Example 2

### Preparation of Phage or Crude Periplasmic Material for Use in ELISAs

ScFvs can be expressed either on the surface of a phage particle or in solution in the bacterial periplasmic space, depending upon the growth conditions used. To induce release of scFv into the periplasm, 96-deepwell plates containing 2×TY media with 0.1% glucose/100 μg/ml ampicillin were inoculated from thawed glycerol stocks (one clone per well) using the QPix2 Colony picker (Genetix) and grown at 37° C. (999 rpm) for ˜4 hours. Cultures were induced with IPTG at a final concentration of 0.02 mM and grown overnight at 30° C. (999 rpm). The contents of the bacterial periplasm (peripreps) were released by osmotic shock. Briefly, plates were centrifuged and pellets were resuspended in 150 μl HEPES periplasmic buffer (50 mM HEPES, pH7.4/0.5 mM EDTA/20% Sucrose), followed by the addition of 150 μl 1:5 HEPES:water and incubated on ice for 30 minutes. Plates were centrifuged and the scFv-containing supernatant was harvested.

To prepare phage expressing scFv on their surface, 96-well plates containing 150 μl 2×TY media with 2% glucose/100 μg/ml ampicillin were inoculated from thawed glycerol stocks as described above and grown at 37° C. (700 rpm) for ˜4 hours. 20 μl of a 1:1000 dilution of helper phage (˜2×10⁸ pfu) was added and the plates incubated for a further hour at 37° C. (300 rpm). Plates were centrifuged and the media was replaced with a kanamycin/non-glucose containing media (2×TY with 50 μg/ml kanamycin and 100 ug/ml ampicillin). Plates were grown overnight at 30° C. (700 rpm) and phage were harvested in the supernatant following centrifugation.

Thirty-one Her2-binding ScFv's were identified by three rounds of screenings as illustrated in FIG. 1. These ScFv's specifically bind to the ECD region of Her2.

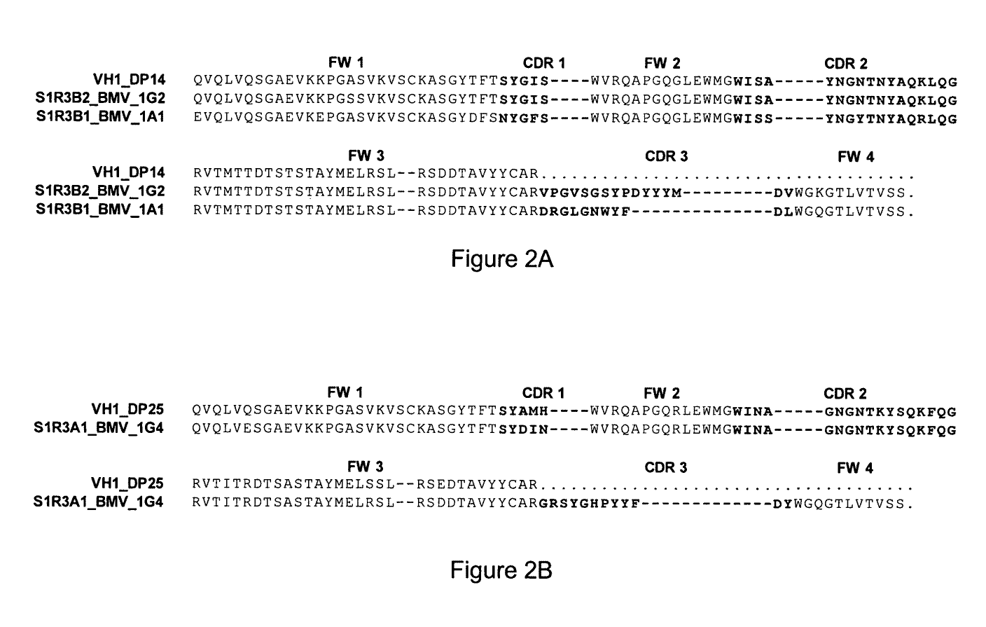

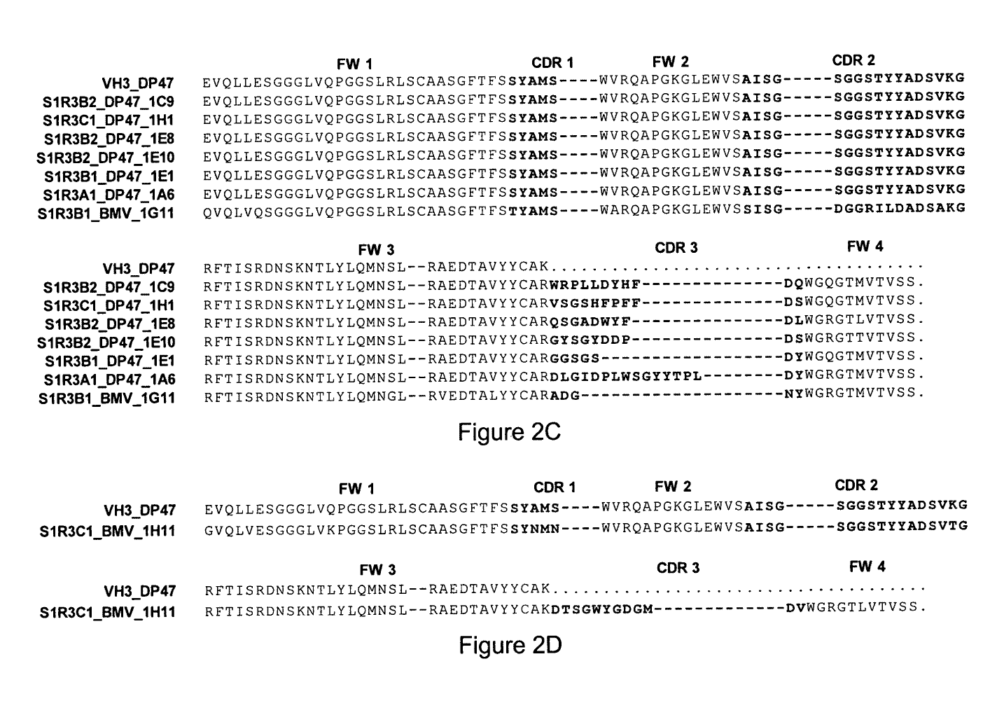

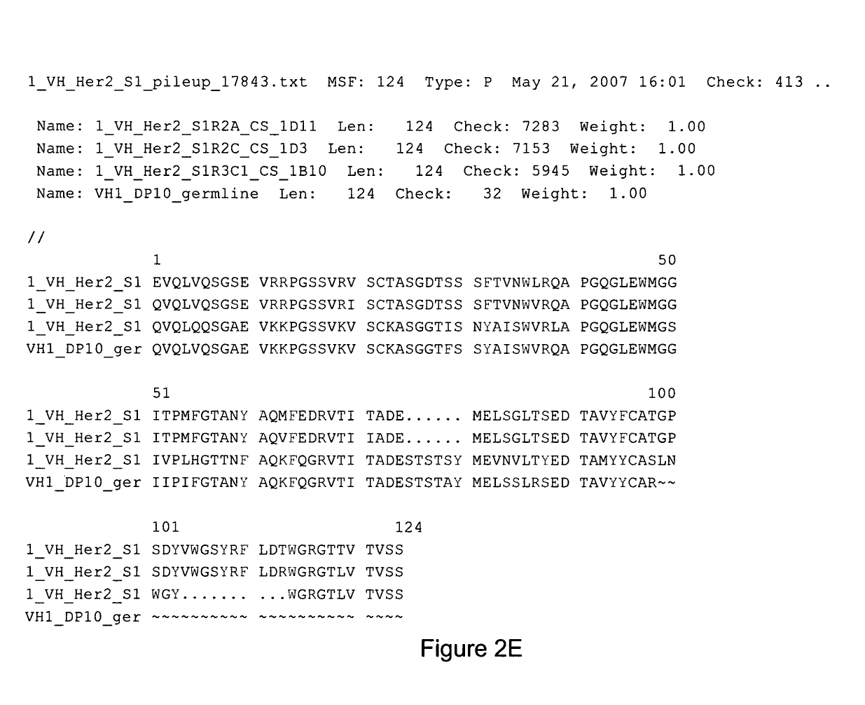

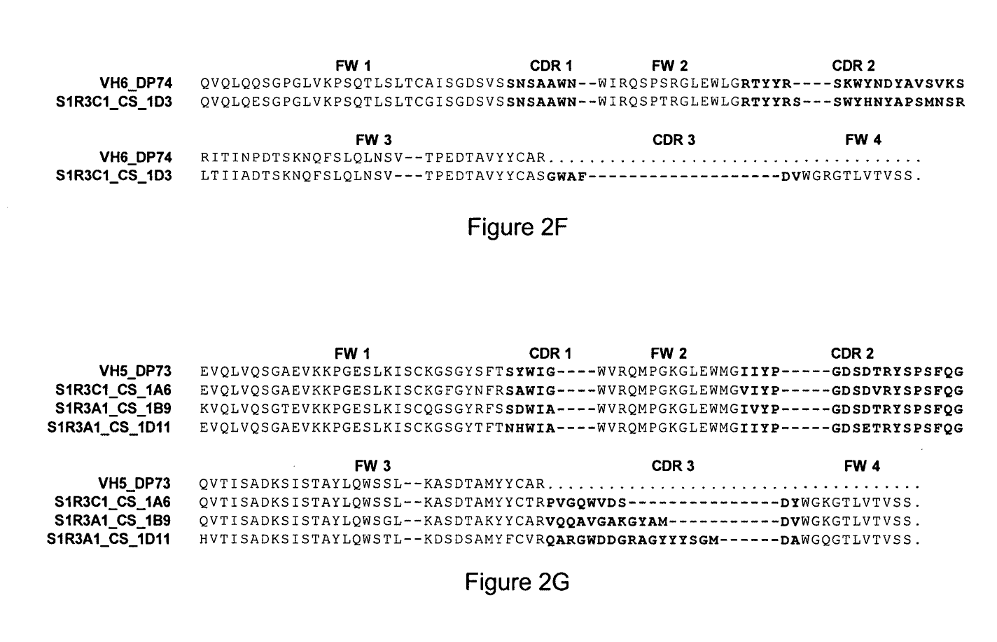

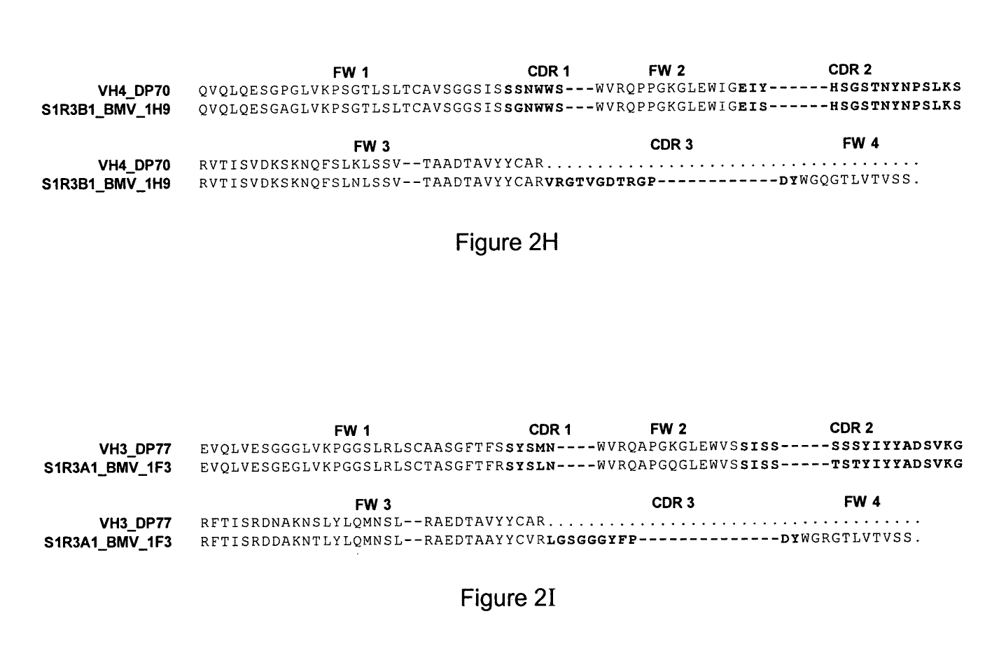

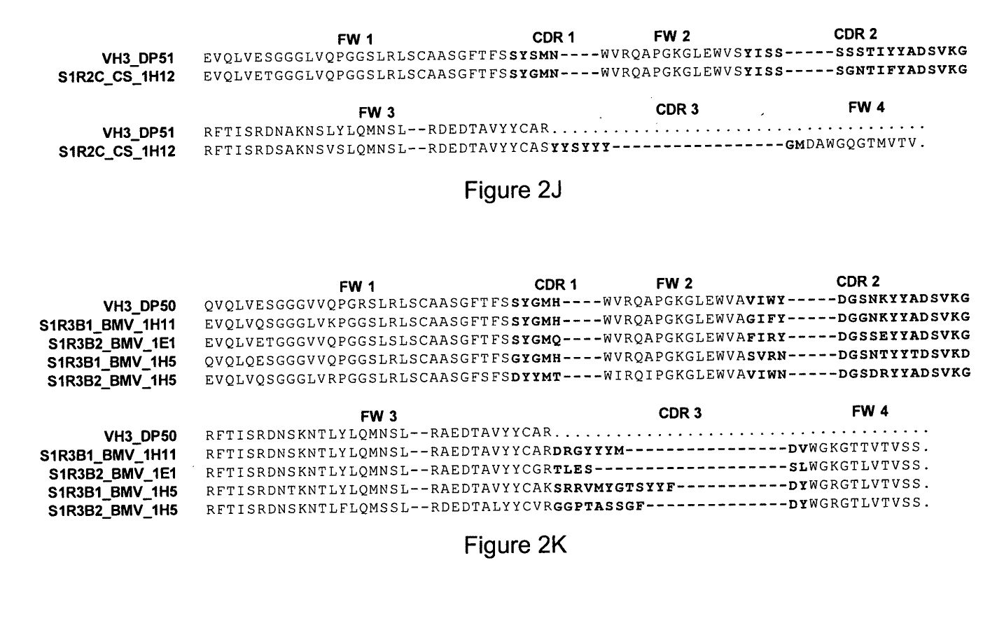

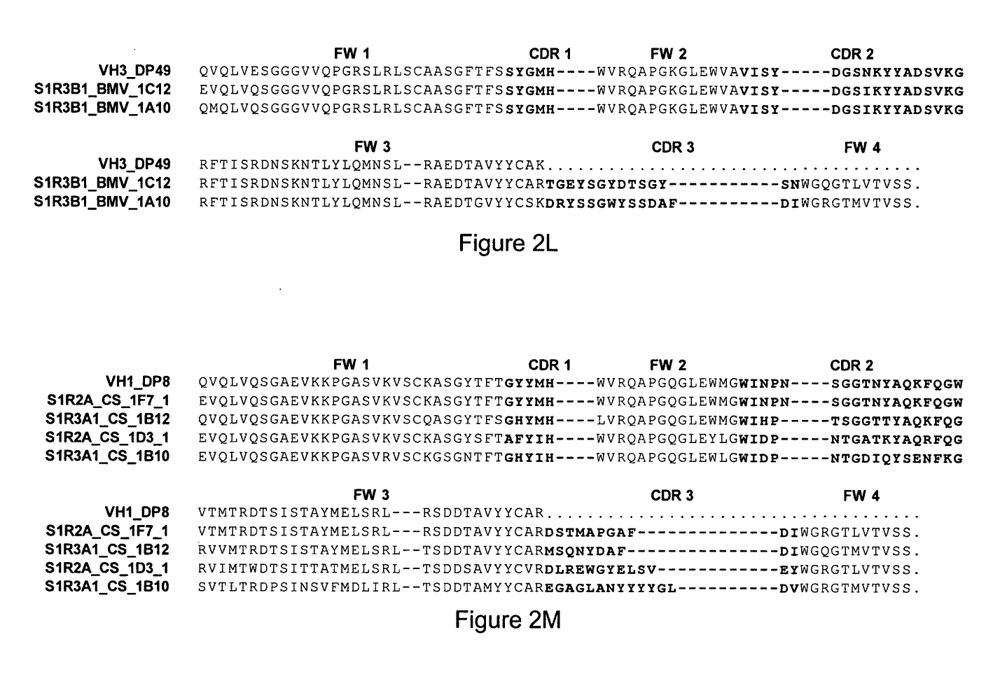

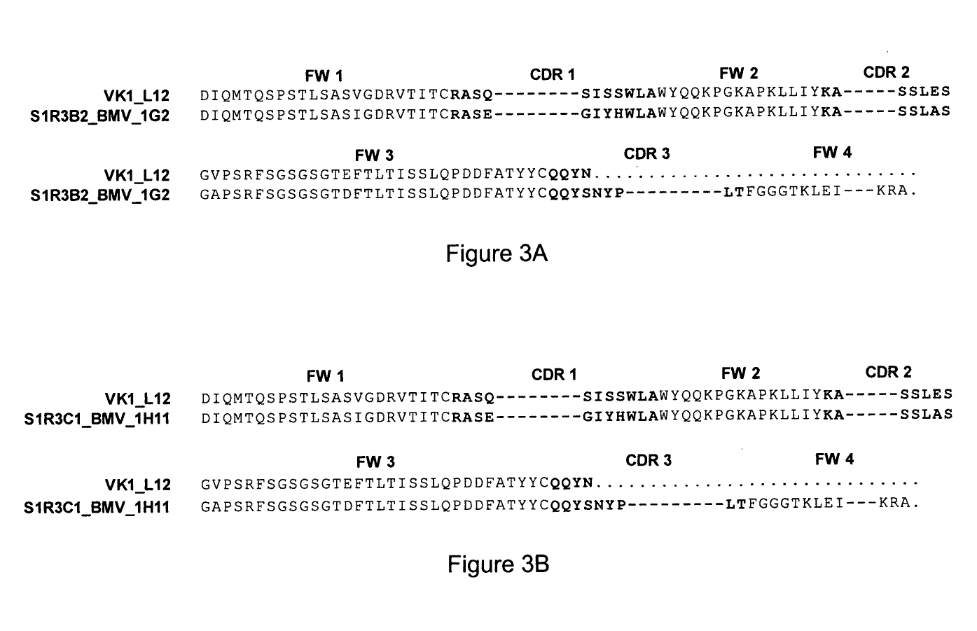

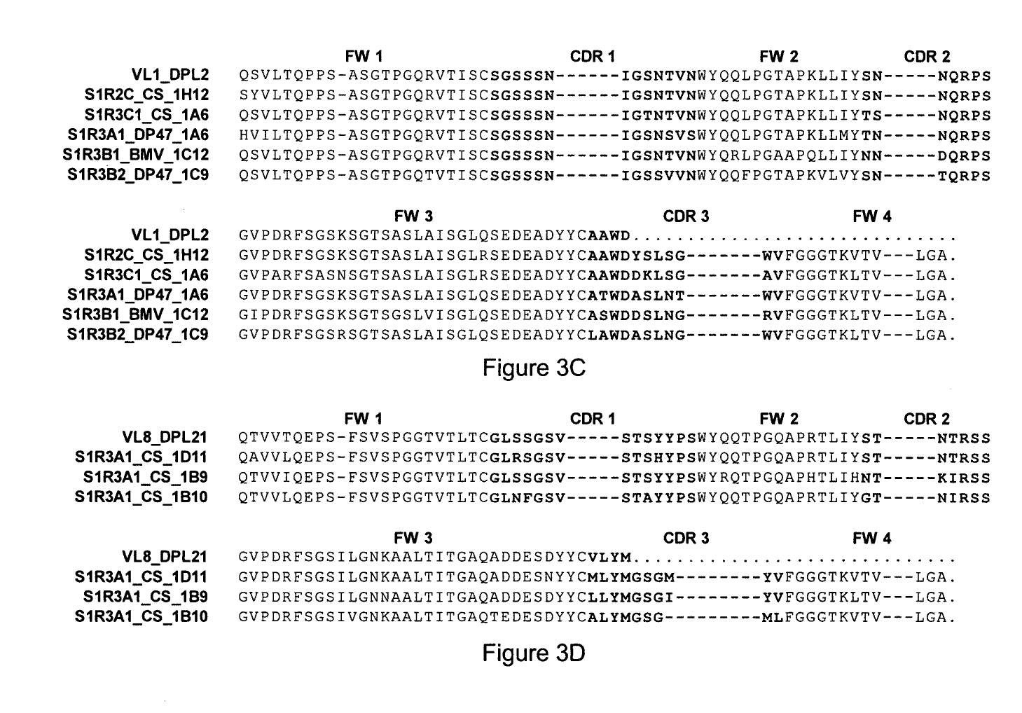

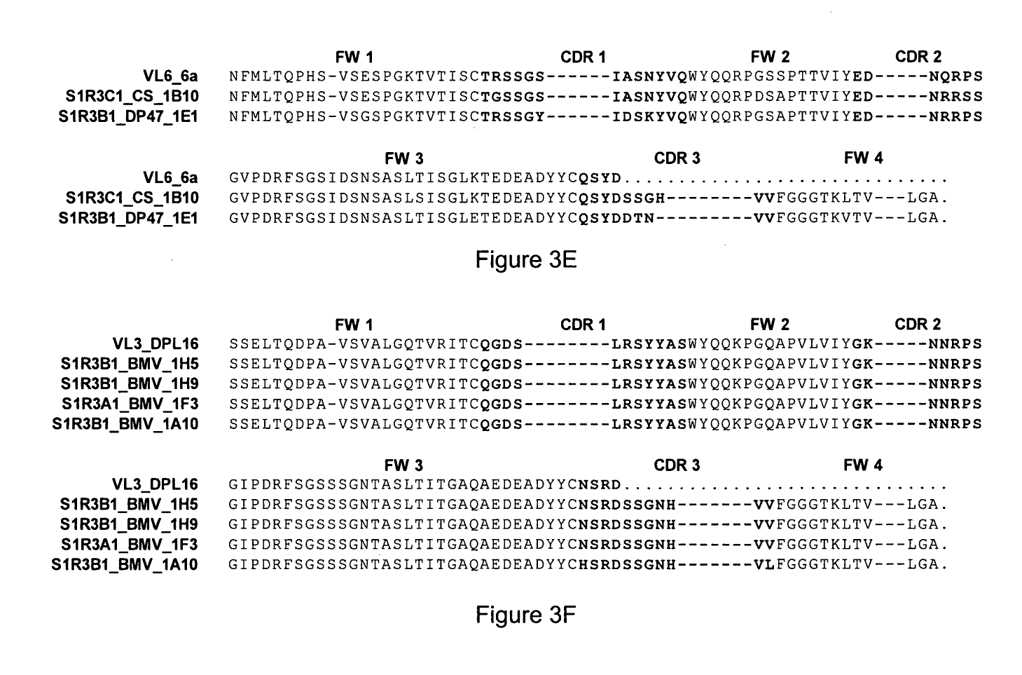

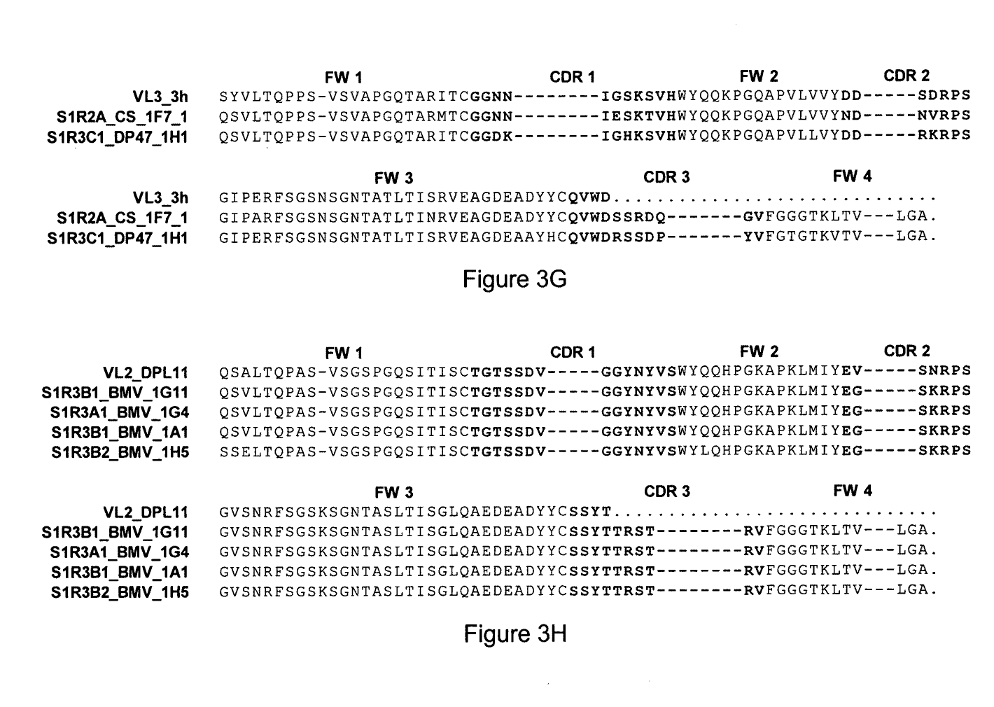

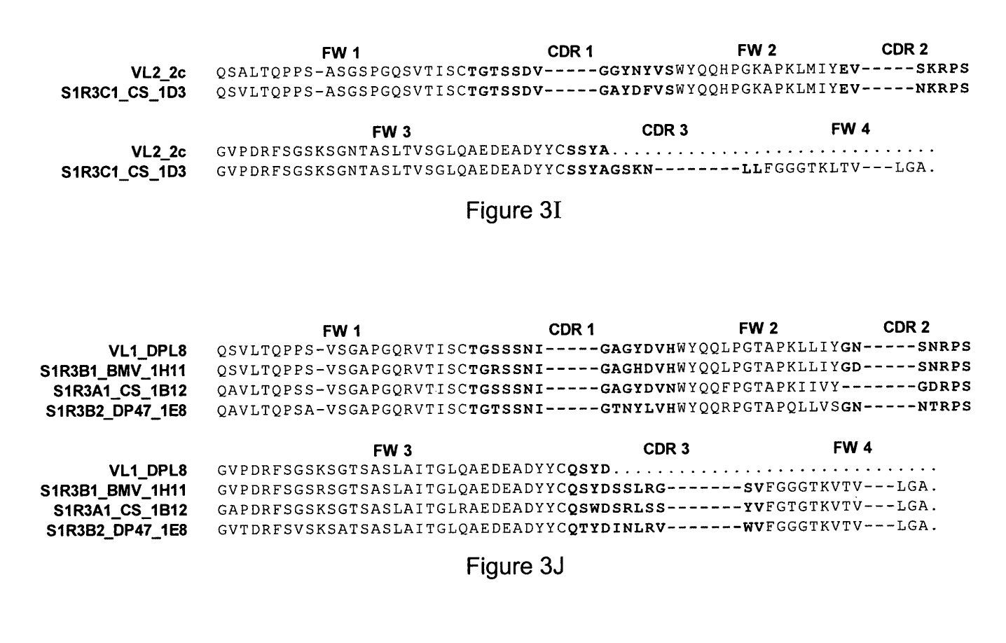

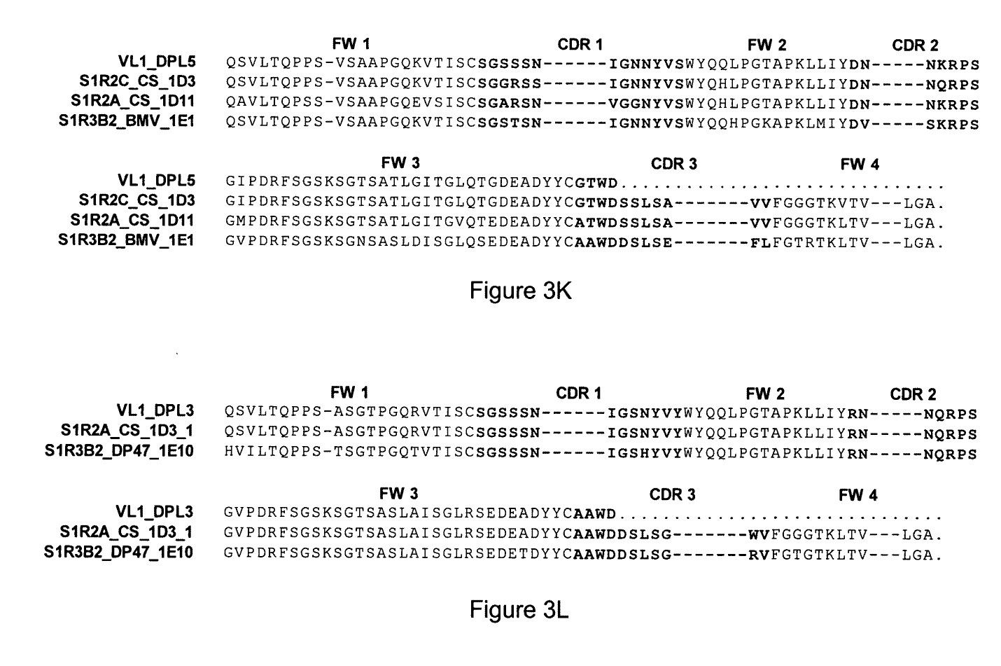

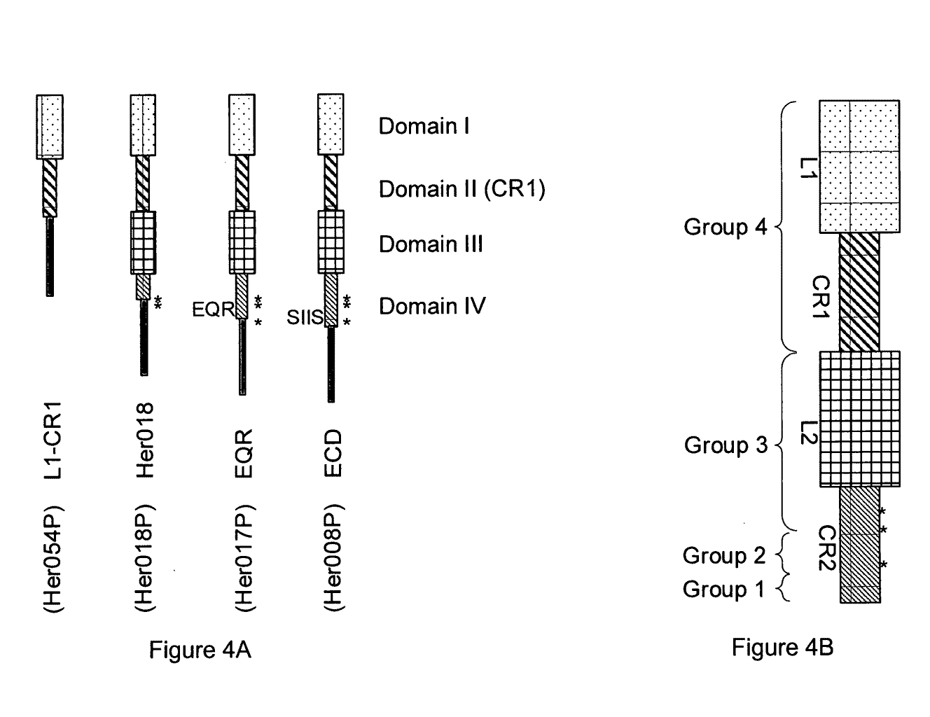

Among these thirty-one Her2-binding ScFv's, fourteen ScFv's were expressed on the surface of a phage particle for the purpose of screening. These ScFv's are: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B1_DP47_{—}1C9, S1R3B2_DP47_{—}1E10, and S1R3C1_CS_{—}1B10 (FIGS. 2 and 3).

The remaining seventeen ScFv's were expressed in bacterial periplasm in soluble form for the purpose of screening: S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, and S1R3B1_BMV_{—}1A1 (FIGS. 2 and 3).

### Example 3

### ELISA to Test Her2 Protein Construct Binding by scFvs Expressed in the E. coli Periplasm, on the Surface of Phage, or in Mammalian Cells as Fc Fusions

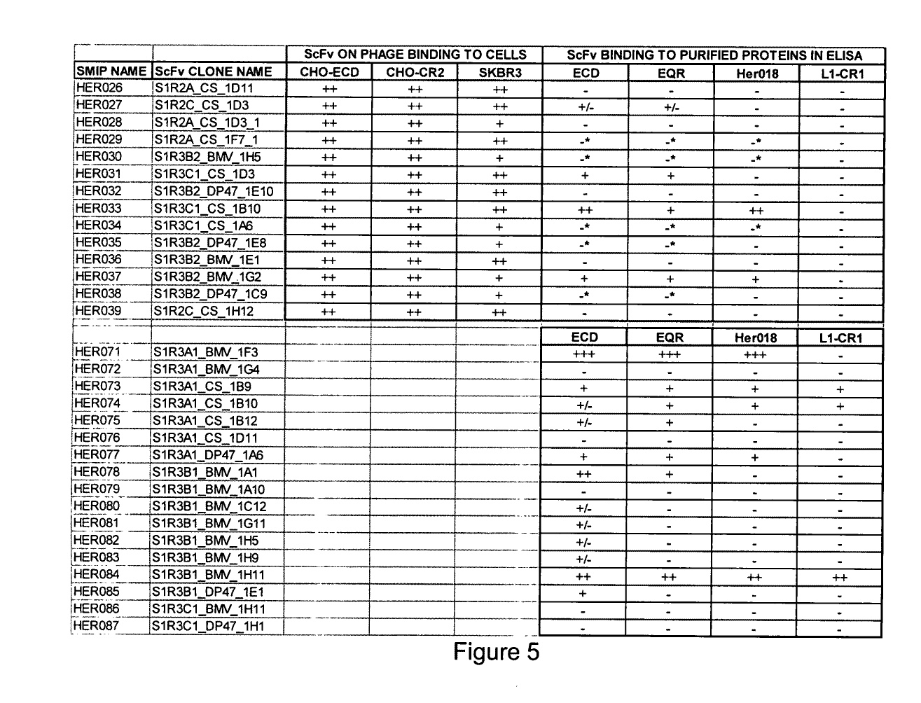

Various Her2-Fc proteins (e.g., Her008P, Her017P, Her018P, etc.) or a negative control murine IgG2a protein were coated overnight at 4° C. on 96-well Nunc Maxisorp at a concentration of 1 ug/ml in PBS. Alternatively, pre-blocked streptavidin-coated plates (Greiner) were coated with biotinylated Her2-Fc proteins for 1 hour at room temperature at a concentration of 1 ug/ml in block buffer (3% skim milk/1% BSA/PBS). Plates were washed three times using PBS and blocked for 1 hour at room temperature in 3% skim milk/1% BSA/PBS. Phage or peripreps were prepared as described above and were blocked for 1 hour at room temperature in an equal volume of 6% skim milk/1% BSA/PBS. Blocked plates were washed five times with PBS and 50 μl/well of blocked phage or periprep were transferred to the appropriate plates and incubated for 1 hour at room temperature. A 1 ug/ml solution of HERCEPTIN® (trastuzumab) (in blocking buffer) was added to well H12 of each plate to serve as a positive control. Plates were washed five times with PBS prior to the addition of a 1:250 dilution of anti-myc peroxidase (Roche), a 1:2500 dilution of anti-M13 peroxidase (Amersham Biosciences) or a 1:5000 or 1:1000 dilution of goat anti-human peroxidase (Southern Biotech) secondary antibody to detect bound scFv, phage, HERCEPTIN® (trastuzumab) or SMIP, respectively. Plates were incubated for a further hour at room temperature and washed seven times with PBS. Signal was developed using TMB, the reaction stopped with H₂SO₄ and the absorbance read at 450 nm on an Envision plate reader (Perkin Elmer). The results of these binding assays are shown in FIG. 5.

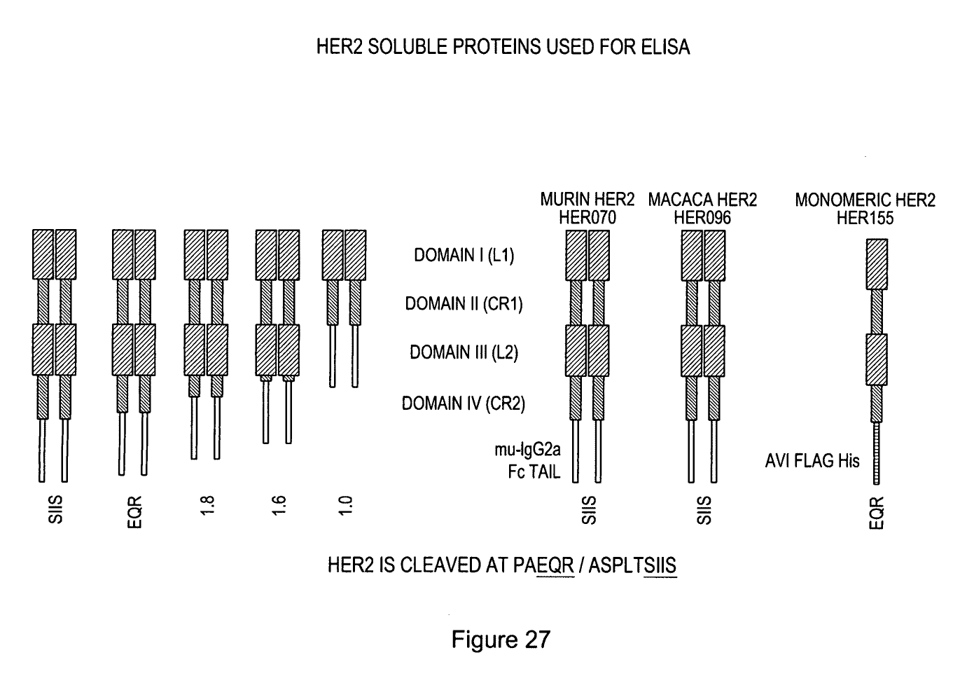

Alternatively, plates were coated with 1 ug/ml of a SMIP (Her030, Her033/Her067, Her018) or antibody (Herceptin®, positive control). SMIPs were used to capture 3-fold serial dilution (9-0 μg/ml) of soluble protein sample (see FIG. 27). Captured soluble protein was detected using 0.1 mg/ml anti-c-Erb B2/c-Neu (Ab-5) mouse mAb (TA-1; binds ECD; Calbiochem) and detected using HRP-conjugated Goat anti-mouse IgG (Fcg Subclass 1 specific; Jackson ImmuonoResearch).

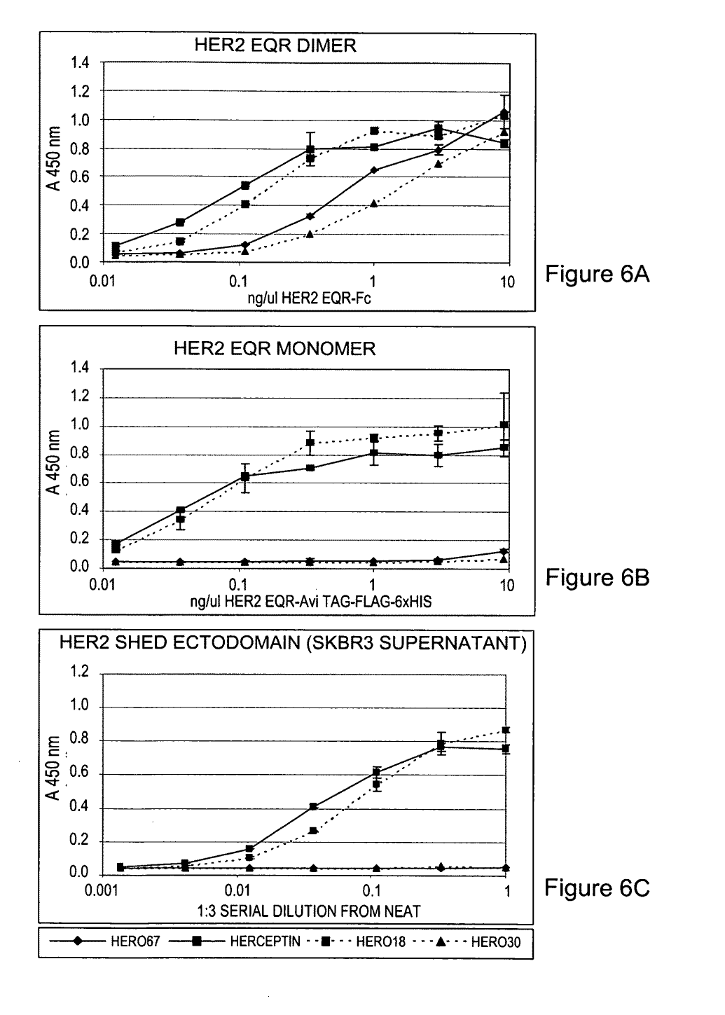

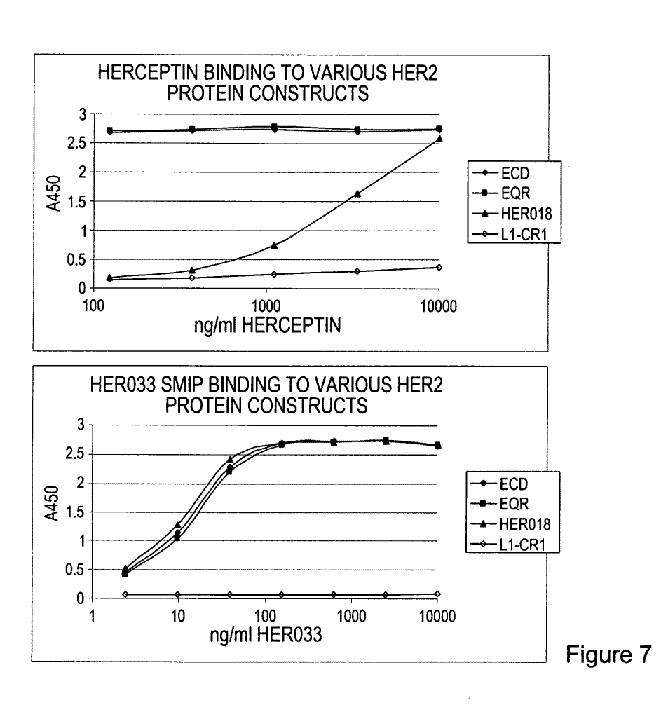

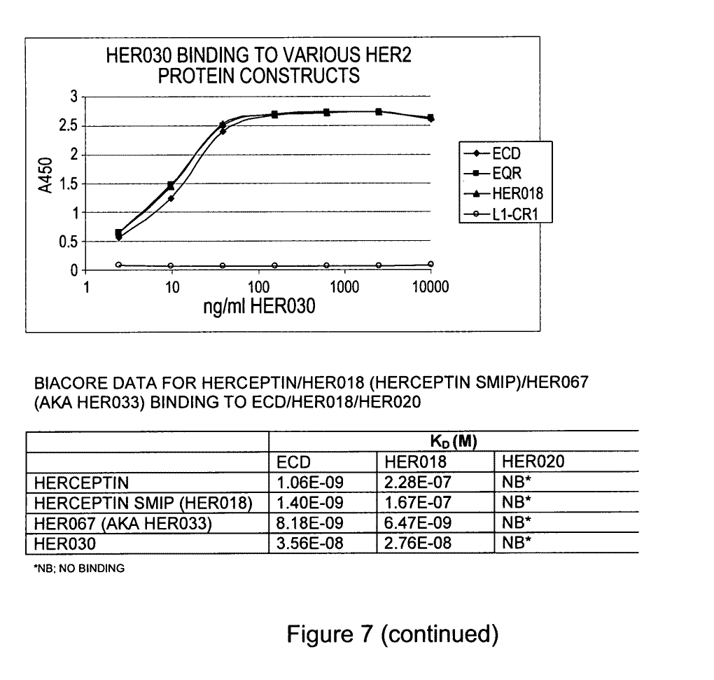

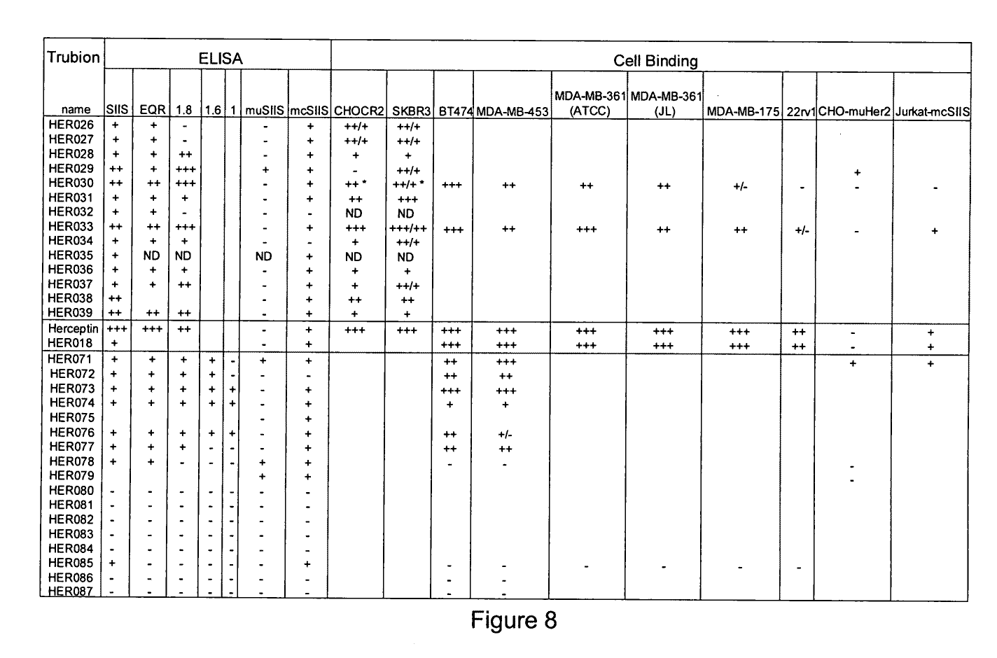

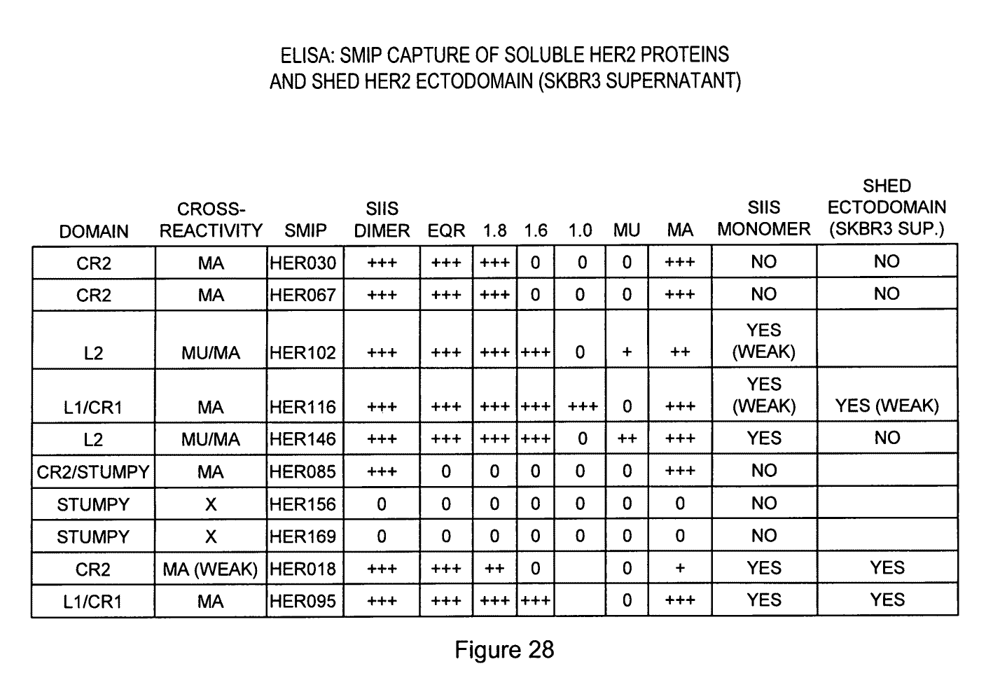

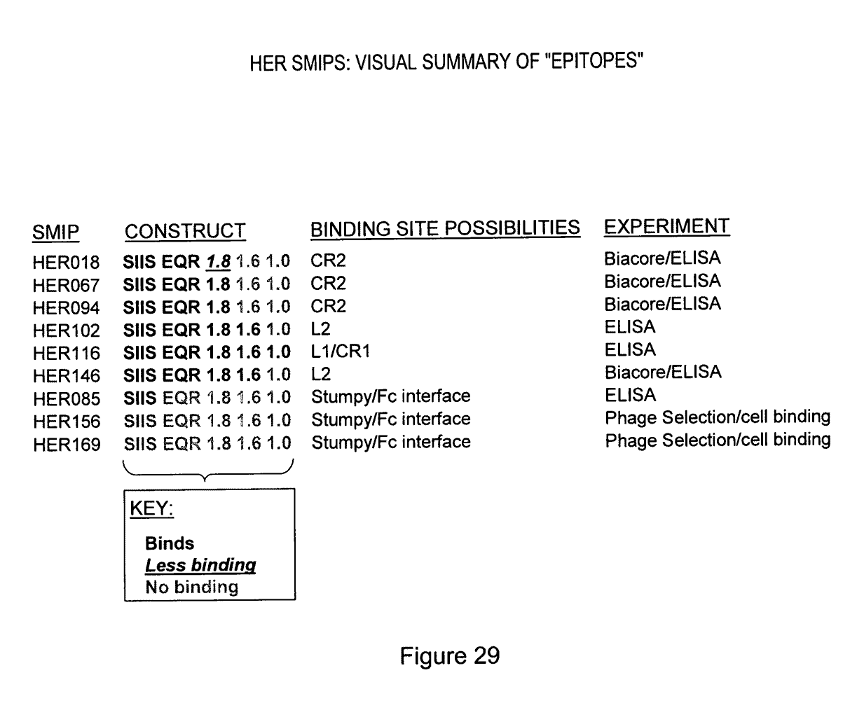

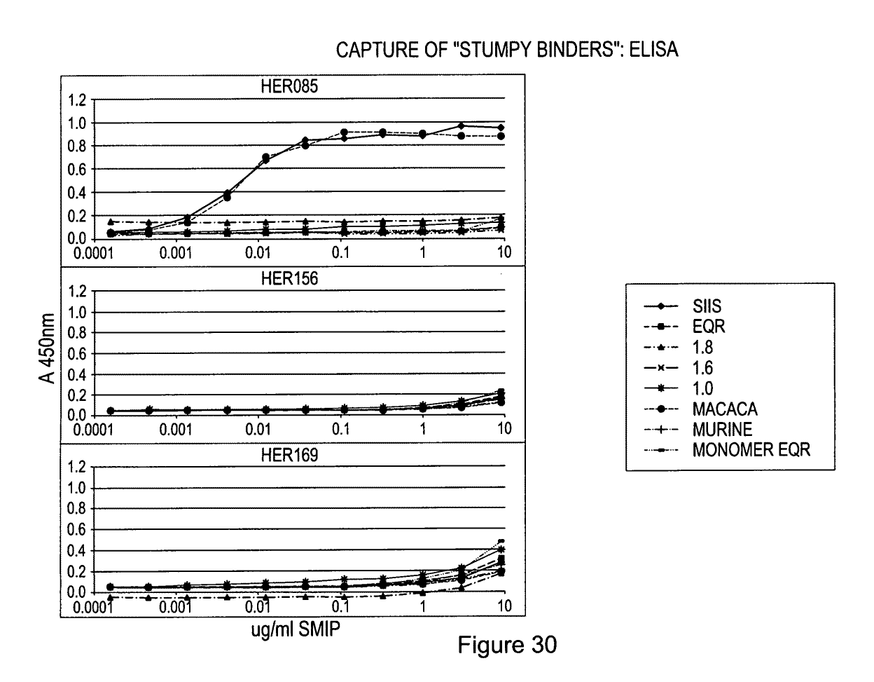

The results of the SMIP binding assays are shown in FIG. 6A-C, FIG. 7A-7D, FIG. 8, AND FIGS. 28-30. In FIG. 8, the binding of HER018, HER026-HER039, and Herceptin® (trastuzumab) and HER018, to Her2 protein constructs was scored as −, +, ++ or +++; the, while the binding of HER071-HER087 to Her2 protein constructs was scored as a − or +. In FIG. 28, the binding of HER SMIPs to Her2 protein constructs was scored as 0, +, ++, or +++, and cross-reactivity and binding domain are shown. FIG. 29 is a graphical summary of the results. HER085 bound soluble full length Her2 ectodomain (ECD) (SIIS dimer) but not soluble Her2 EQR(SIIS lacking membrane proximal amino acids ASPLTSIIS). This indicated that HER085 binding domain required “stumpy” amino acids ASPLTSIIS. HER156 and HER169 did not bind soluble full length HER2ECD (SIIS dimer) which includes the “stumpy” peptide although they bound the synthetic “stumpy” ASPLTSIIS peptide on which they were selected. This suggests that the “stumpy” peptide in Her2 SIIS was not presented in a form recognized by HER156 and HER169, because HER156 and HER169.

The results are summarized in the following Table.

| | Binding | Human V_{H} | Human V_{L} germline |
|---|---|---|---|
| mAB | phenotype | germline gene | gene |
| S1R3B1_DP47_3A2 | Group 2 | Vh3_DP-47_(3-23) | Vlambda2_DPL11_(2a2) |
| S1R3A1_DP47_11B7 | Group 3 | Vh3_DP-47_(3- | Vlambda1_DPL8_(1e) |
| | | 23) | |
| S1R3A1_DP47_11D1 | Group 3 | Vh3_DP-47_(3- | Vlambda3_DPL16_(3l) |
| | | 23) | |
| S1R3A1_DP47_7F3 | Group 3 | Vh3_DP-47_(3- | Vlambda1_DPL8_(1e) |
| | | 23) | |
| S1R2B_DP47_4E3 | Group 3 | Vh3_DP-47_(3- | Vlambda1_DPL3_(1g) |
| | | 23) | |
| S1R3C1_DP47_2G2 | Group 3 | Vh3_DP-47_(3- | Vlambda1_DPL2_(1c) |
| | | 23) | |
| S1R3A1_DP47_11H6 | Group 3 | Vh3_DP-47_(3- | Vlambda1_DPL3_(1g) |
| | | 23) | |
| S1R3A1_BMV_3B1 | Group 3 | Vh3_DP-49_(3- | Vlambda2_DPL11_(2a2) |
| | | 30.5) | |
| S1R3A1_DP47_6B9 | Group 3 | Vh3_DP-47_(3- | Vlambda1_DPL8_(1e) |
| | | 23) | |
| S1R2A_CS_10B8 | Group 3 | Vh1_DP-7_(1- | Vlambda1_DPL2_(1c) |
| | | 46) | |
| S1R3A1_DP47_7A6 | Group 3 | Vh3_DP-47_(3- | Vlambda1_DPL8_(1e) |
| | | 23) | |
| S1R3B2_DP47_2G3 | Group 3 | Vh3_DP-47_(3- | Vlambda3_DPL16_(3l) |
| | | 23) | |
| S1R2B_CS_6H11 | Group 3 | Vh5_DP-73_(5- | Vlambda8_DPL21_(8a) |
| | | 51) | |
| S1R3A1_DP47_10G1 | Group 3 | Vh3_DP-47_(3- | Vlambda1_DPL8_(1e) |
| | | 23) | |
| S1R3A1_DP47_7C1 | Group 3 | Vh3_DP-47_(3- | Vlambda1_DPL8_(1e) |
| | | 23) | |
| S1R2A_DP47_5D6 | Group 3 | Vh3_DP-47_(3- | Vk3_DPK22_(A27) |
| | | 23) | |
| S1R3A1_DP47_11F6 | Group 3 | Vh3_DP-47_(3- | Vlambda1_DPL5_(1b) |
| | | 23) | |
| S1R3A1_DP47_11D3 | Group 3 | Vh3_DP-47_(3- | Vlambda1_DPL3_(1g) |
| | | 23) | |
| S1R3A1_CS_8A8 | Group 3 | Vh5_DP-73_(5- | Vlambda8_DPL21_(8a) |
| | | 51) | |
| S1R3A1_BMV_5D10 | Group 3 | Vh3_DP-47_(3- | Vk1_L12 |
| | | 23) | |
| S1R3A1_DP47_11C1 | Group 3 | Vh3_DP-47_(3- | Vlambda3_DPL16_(3l) |
| | | 23) | |
| S1R3A1_DP47_4E1 | Group 3 | Vh3_DP-47_(3- | Vlambda1_DPL8_(1e) |
| | | 23) | |
| S1R3A1_DP47_10E1 | Group 3 | Vh3_DP-47_(3- | Vlambda1_DPL2_(1c) |
| | | 23) | |
| S1R3A1_CS_11C3 | Group 3 | Vh1_DP-14_(1- | Vlambda3_DPL16_(3l) |
| | | 18) | |
| S1R3A1_CS_13H11 | Group 3 | Vh1_DP- | Vlambda1_DPL5_(1b) |
| | | 8.75_(1-02) | |
| S1R3A1_CS_2D9 | Group 4 | Vh5_DP-73_(5- | Vk1_L12 |
| | | 51) | |
| S1R2A_CS_3D4 | Group 4 | Vh1_DP- | Vlambda1_DPL8_(1e) |
| | | 8.75_(1-02) | |
| S1R3A1_DP47_2H6 | Group 4 | Vh3_DP-47_(3- | Vlambda3_DPL23_(3r) |
| | | 23) | |
| S1R3A1_DP47_4G1 | Group 4 | Vh3_DP-47_(3- | Vlambda3_3h |
| | | 23) | |
| S1R2A_DP47_3C1 | Group 4 | Vh3_DP-47_(3- | Vlambda3_DPL23_(3r) |
| | | 23) | |
| S1R3A1_DP47_7B2 | Group 4 | Vh3_DP-47_(3- | Vk1_DPK6_(L19) |
| | | 23) | |
| S1R3B2_DP47_4E2 | Group 4 | Vh3_DP-47_(3- | Vlambda1_DPL2_(1c) |
| | | 23) | |
| S1R3A1_CS_16C2 | Group 4 | Vh3_DP-47_(3- | Vlambda3_DPL16_(3l) |
| | | 23) | |
| S1R3A1_CS_11E5 | Group 4 | Vh5_DP-73_(5- | Vlambda8_DPL21_(8a) |
| | | 51) | |
| S1R3A1_CS_16D7 | Group 4 | Vh5_DP-73_(5- | Vlambda8_DPL21_(8a) |
| | | 51) | |
| S1R2A_CS_10B10 | Group 4 | Vh1_DP- | Vlambda8_DPL21_(8a) |
| | | 8.75_(1-02) | |
| S1R3A1_CS_15C2 | Group 4 | Vh4_DP-70_(4- | Vlambda3_DPL16_(3l) |
| | | 04) | |
| S1R3A1_CS_9C1 | Group 4 | Vh4_DP-67_(4- | Vlambda1_DPL8_(1e) |
| | | b) | |
| S1R2A_CS_5A1 | Group 4 | Vh3_DP-47_(3- | Vlambda1_DPL2_(1c) |
| | | 23) | |
| S1R2A_CS_8C8 | Group 4 | Vh5_DP-73_(5- | Vlambda8_DPL21_(8a) |
| | | 51) | |
| S1R3A1_CS_13H5 | Group 4 | Vh5_DP-73_(5- | Vlambda8_DPL21_(8a) |
| | | 51) | |
| S1R2B_CS_5E9 | Group 4 | Vh5_DP-73_(5- | Vlambda8_DPL21_(8a) |
| | | 51) | |
| S1R3A1_CS_8F9 | Group 4 | Vh5_DP-73_(5- | Vlambda8_DPL21_(8a) |
| | | 51) | |
| S1R3A1_CS_14B5 | Group 4 | Vh5_DP-73_(5- | Vlambda8_DPL21_(8a) |
| | | 51) | |
| S1R2A_CS_9E10 | Group 4 | Vh5_DP-73_(5- | Vlambda8_DPL21_(8a) |
| | | 51) | |
| S1R3A1_CS_7A10 | Group 4 | Vh5_DP-73_(5- | Vlambda8_DPL21_(8a) |
| | | 51) | |
| S1R3A1_BMV_6H7 | Group 4 | Vh3_DP-46_(3- | Vk1_L12 |
| | | 30.3) | |
| S1R3A1_CS_12A11 | Group 4 | Vh5_DP-73_(5- | Vlambda8_DPL21_(8a) |
| | | 51) | |
| S1R3A1_CS_13D12 | Group 4 | Vh5_DP-73_(5- | Vlambda8_DPL21_(8a) |
| | | 51) | |
| S1R3A1_CS_7A8 | Group 4 | Vh4_DP-79_(4- | Vlambda8_DPL21_(8a) |
| | | 39) | |
| S1R2A_CS_2C9 | Group 4 | Vh1_DP-7_(1- | Vk3_DPK21_(L2) |
| | | 46) | |
| S1R3A1_CS_12D1 | Group 4 | Vh3_3-73 | Vlambda1_DPL8_(1e) |
| S1R2A_CS_7D4 | Group 4 | Vh1_DP-5_(1- | Vlambda8_DPL21_(8a) |
| | | 24) | |
| S1R3A1_CS_15B8 | Group 4 | Vh5_DP-73_(5- | Vlambda8_DPL21_(8a) |
| | | 51) | |
| S6R3_DP47_1A10 | Group 1 | 3-23 (DP47) | Vλ 1c (DPL2) |
| S6R2_DP47_1E11 | Group 1 | 3-23 (DP47) | Vλ 1c (DPL2) |
| S5R2_DP47_1H11 | Group 1 | 3-23 (DP47) | Vλ 1c (DPL2) |
| S6R3_CS_1G5 | Group 1 | 4-39 | Vλ 1g (DPL3) |
| | | (DP79) | |
| S6R2_DP47_1H11 | Group 1 | 3-23 (DP47) | Vλ 1b (DPL5) |
| S5R3_DP47_1A10 | Group 1 | 3-23 (DP47) | Vλ 1e (DPL8) |
| S5R2_DP47_1D11 | Group 1 | 3-23 (DP47) | Vλ 1e (DPL8) |
| S5R2_CS_1A8 | Group 1 | 5-51 (DP73) | Vλ 1e (DPL8) |
| S6R3_CS_1B7 | Group 1 | 5-51 (DP73) | Vλ 1e (DPL8) |
| S6R2_CS_1E5 | Group 1 | 1-02 (DP-8,75) | Vλ 2e2 (DPL11) |
| S6R3_BMV_1C2 | Group 1 | 7-4.1 (DP21) | Vλ 2a2 (DPL11) |
| S5R2_DP47_1B10 | Group 1 | 3-23 (DP47) | Vλ 3l (DPL16) |
| S6R3_DP47_1C12 | Group 1 | 3-23 (DP47) | Vλ 3l (DPL16) |
| S5R2_DP47_1D10 | Group 1 | 3-23 (DP47) | Vλ 3l (DPL16) |
| S6R3_DP47_1H9 | Group 1 | 3-23 (DP47) | Vλ 3l (DPL16) |

### Example 4

### ELISA to Measure Binding of scFvs (Expressed in the Periplasm or on the Surface of Phage) to Her2-Expressed Cells

2×10⁴ CHOK1 cells/well were seeded in a 96-well tissue culture plate on Day 1 and incubated at 37° C./5% CO₂ for 2-4 days until a confluent monolayer was observed. Cells were washed five times with PBS (+Ca/Mg ions) and blocked for 1 hour at room temperature with 3% skim milk/1% BSA/PBS (+Ca/Mg ions). Phage or peripreps were prepared as described above and were blocked for 1 hour at room temperature in an equal volume of 6% skim milk/1% BSA/PBS (+Ca/Mg ions). Blocked plates were washed five times with PBS (+Ca/Mg ions) and 50 μl/well of blocked phage or periprep were transferred to the appropriate plates and incubated for 1 hour at room temperature. A 1 ug/ml solution of HERCEPTIN® (trastuzumab) (in blocking buffer) was added to well H12 of each plate to serve as a positive control. Plates were washed five times with PBS (+Ca/Mg ions) prior to the addition of a 1:250 dilution of anti-myc peroxidase (Roche), a 1:2500 dilution of anti-M13 peroxidase (Amersham Biosciences) or a 1:5000 dilution of goat anti-human (Southern Biotech) secondary antibody to detect bound scFv, phage or HERCEPTIN® (trastuzumab) respectively. Plates were incubated for a further hour at room temperature and washed ten times with PBS (+Ca/Mg ions). Signal was developed using TMB, the reaction stopped with H₂SO₄ and the absorbance read at 450 nm on an ENVISION plate reader (Perkin Elmer). The results of these binding assays are shown in FIG. 5.

Alternatively, we tested anti-HER2SMIP binding to cell line including JIMT-1, SKBR3, BT474, 22rv1, MDA-MB-175, MDA-MB-453, MDA-MB-361 (ATCC), MDA-MB-361 (JL), and Ramos (Her2⁻/CD20⁺ control). Controls used included Herceptin® (trastuzumab), Rituxan® (anti-CD20 mAb rituximab), and CD20-SMIP(2LM20-4 SCCP).

Each well of a 6 well plate was seeded with 2×10⁵ cells and incubated overnight at 37° C./5% CO₂. Cells were then treated with antibody or SMIP (at 10 ug/ml final) (in triplicate) and incubated for another 24 or 48 hours. After incubation, the cells were pulsed with 50 uM BrdU (Sigma) for 30 minutes at 37° C., the media was removed, and the cells were treated with trypsin (except Ramos) and then 3-3.5×10⁵ cells per well were stained in 100 μl Staining Buffer in the presence or absence of a SMIP or antibody one of three different concentrations (ranging from 200 nM to 0.27 nM). The SMIP or antibody treatment was removed and the cells were washed three times with PBS, pH 7.2-7.4 with 0.1% TWEEN®-20 (PBS-T). A secondary antibody (5 ug/ml Alexa Fluor 488-conjugated Goat anti-Human IgG; Molecular Probes #A-11013) was then added and incubated for 1-2 hours at room temperature. The secondary antibody was removed and the cells washed again three times with PBS-T. The cells were then fixed in 1% paraformaldehyde in Staining Buffer and analyzed 1 hour to 1 day later.

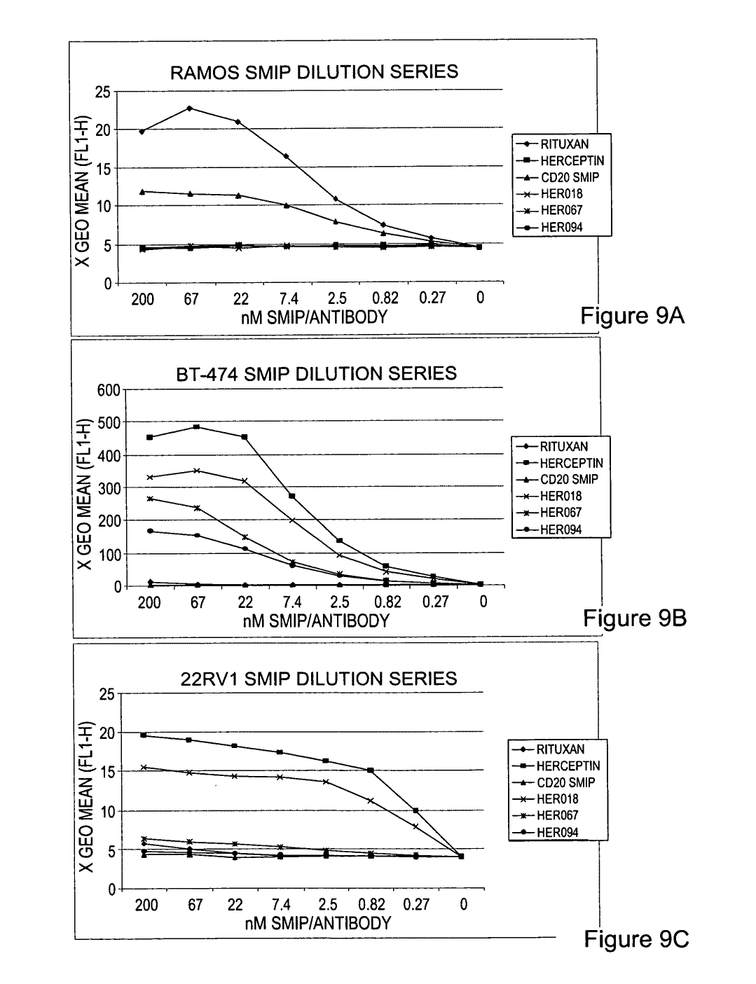

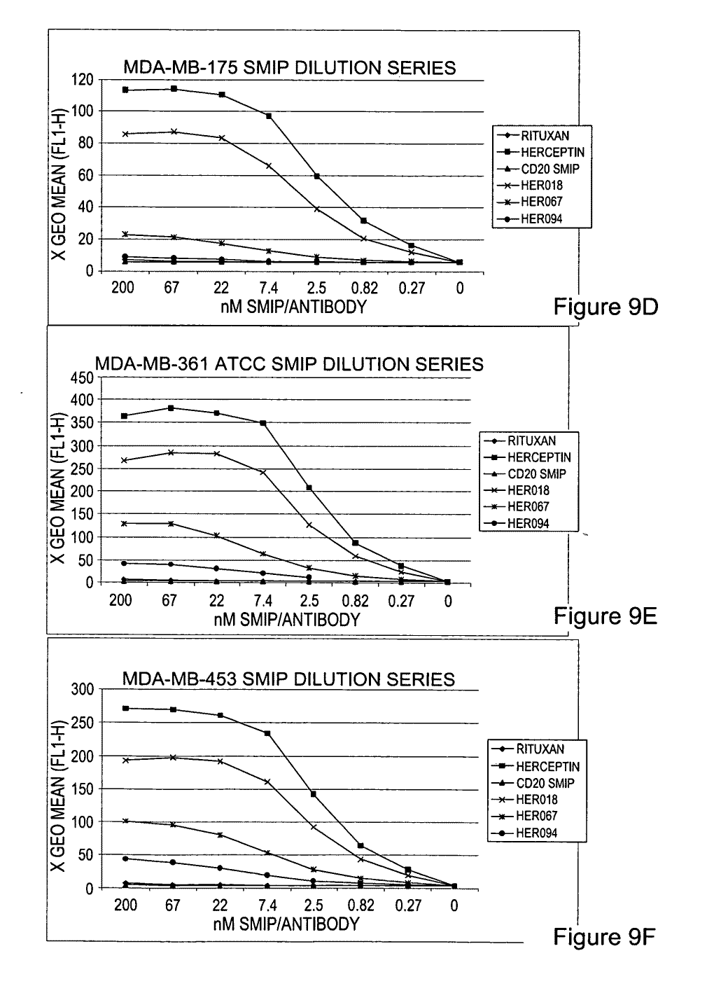

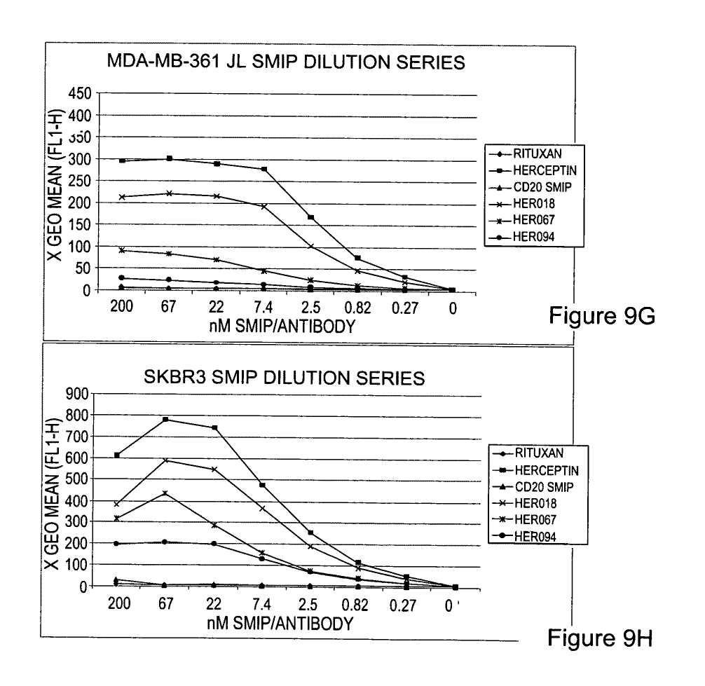

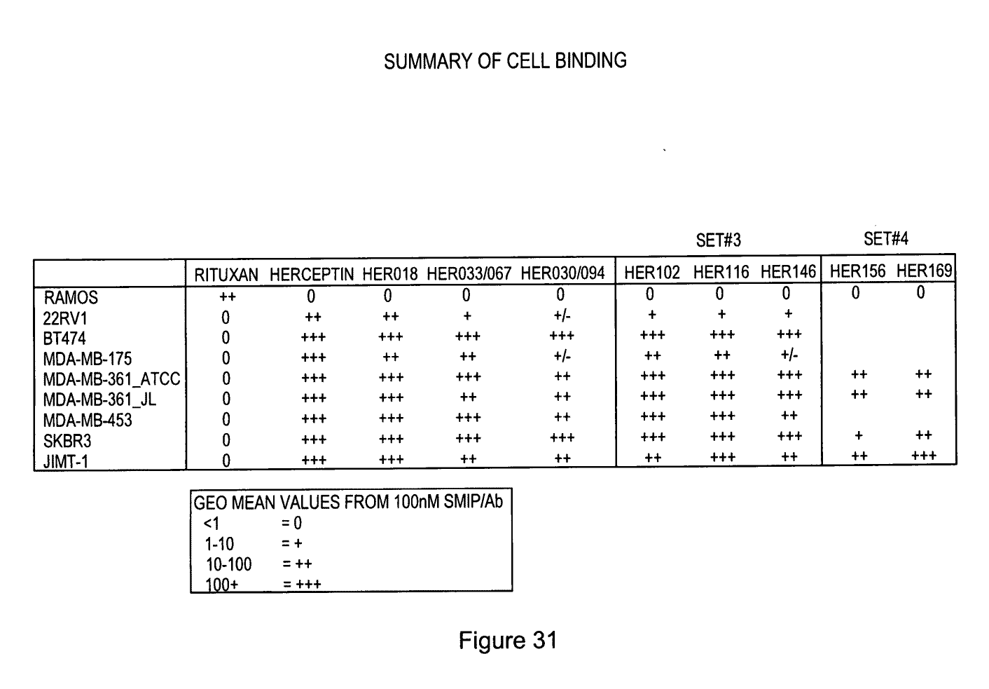

The results of these binding assays are shown in FIG. 8 and FIGS. 9A-9H and are summarized in FIG. 31. (In FIG. 9E, 0.82 nM HER094 data not collected due to mechanical error.) As shown in the Figures, SMIPs maintain a similar staining pattern regardless of the amount of HER2 on the cell surface and the other ErbB receptors/ligands expressed by the cell lines (relative surface staining for ErbB1, Her2, Erb3 and production of ligand by cell lines is not shown). In these experiments, the SMIP staining pattern is HER116>HER067>HER012>HER146>HER094. Additionally, HER116 binding to BT-474 cells changes the FSC vs SSC profile of BT-474 cells in a manner that suggests clumping.

JIMT-1 Binding Assay

To investigate anti-HER2SMIP binding to JIMT-1 cells we dissociated JIMT-1 cells with trypsin or non-enzymatic CellStripper™ (cellgro Mediatech #25-056-C1). JIMT-1 ErbB2 epitopes may be partially blocked by MUC4 (Peter Nagy, Elza Friedländer, Minna Tanner, Anita I. Kapanen, Kermit L. Carraway, Jorma Isola, and Thomas M. Jovin. Decreased Accessibility and Lack of Activation of ErbB2 in JIMT-1, a Herceptin-Resistant, MUC4-Expressing Breast Cancer Cell Line. Cancer Res 65(2): 473-482, 2005). We then washed cells three times after dissociation and stained them in duplicate with 200 nM Antibody/SMIP (200 nM=22 μg/mL SMIP). We performed secondary staining with 5 μg/mL Alexa Fluor 488-conjugated Goat anti-Human IgG (Molecular Probes #A-11013).

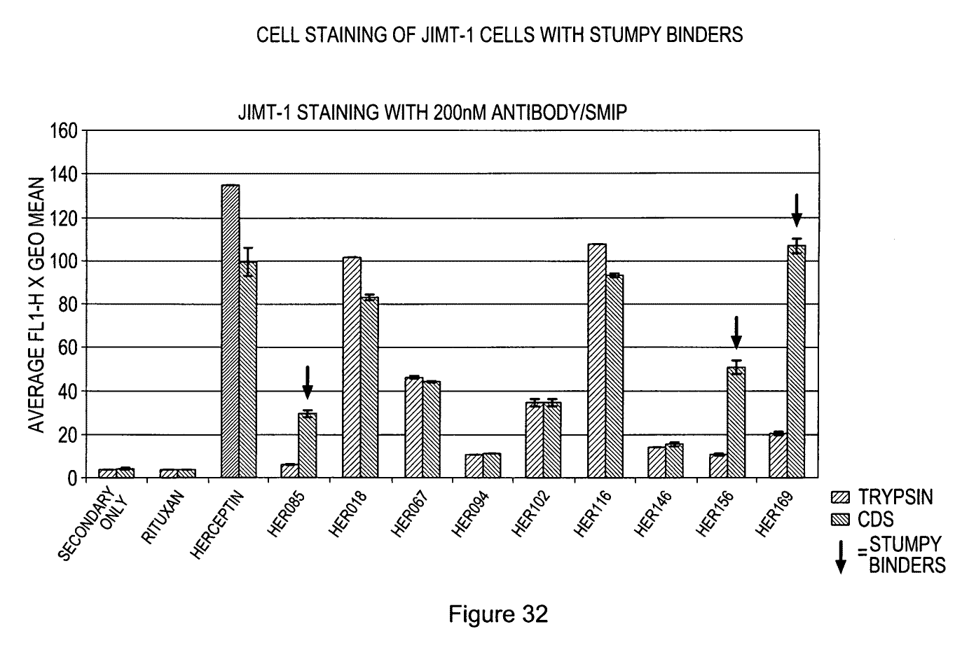

The results are shown in FIG. 32. As shown in FIG. 32, HER085, HER156 and HER169, all “stumpy” binders bind to multiple cell lines harvested by non-enzymatic means better than to trypsinized cells, suggesting that the epitope recognized by these SMIPS may be sensitive to trypsin whereas other SMIPs and HERCEPTIN® showed equal or better binding on trypsinized cells. This sensitivity was not likely to be direct as trypsin did not affect the ability of other SMIPs to bind to the full length Her2 ECD (which contains the “stumpy” peptide ASPLTSIIS). The sensitivity may be due to trypsin cleavage of other molecules that are needed for the presentation/exposure of the “stumpy” peptide or the maintenance of Her2 p95 (“stumpy”) on the cell surface.

Enzymatic Vs Non-Enzymatic Preparation

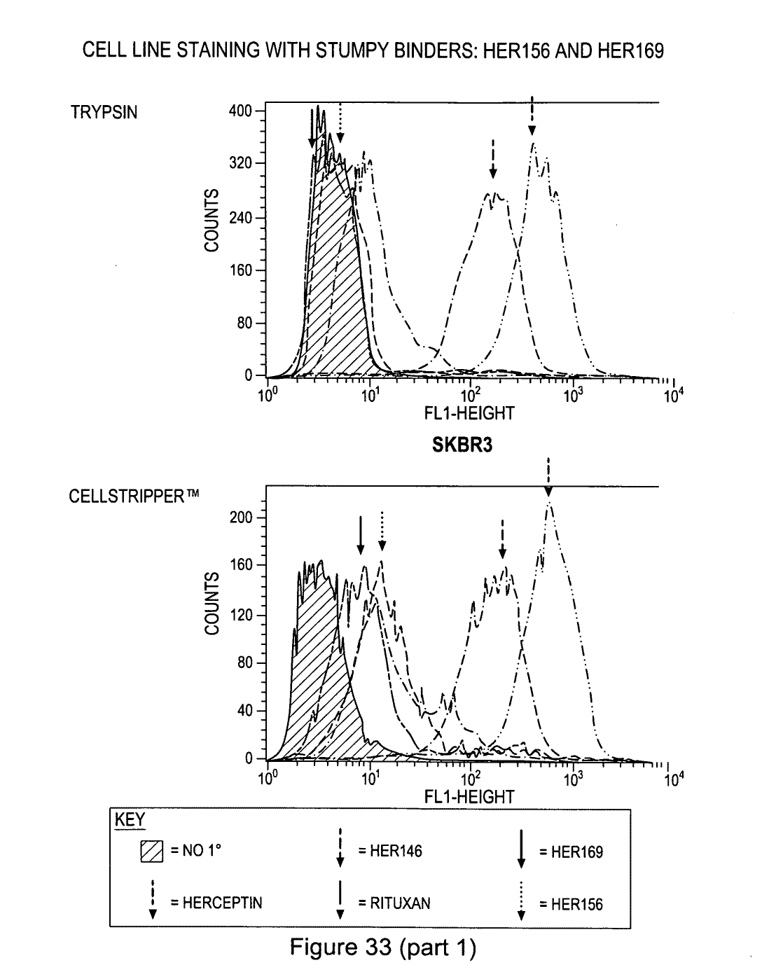

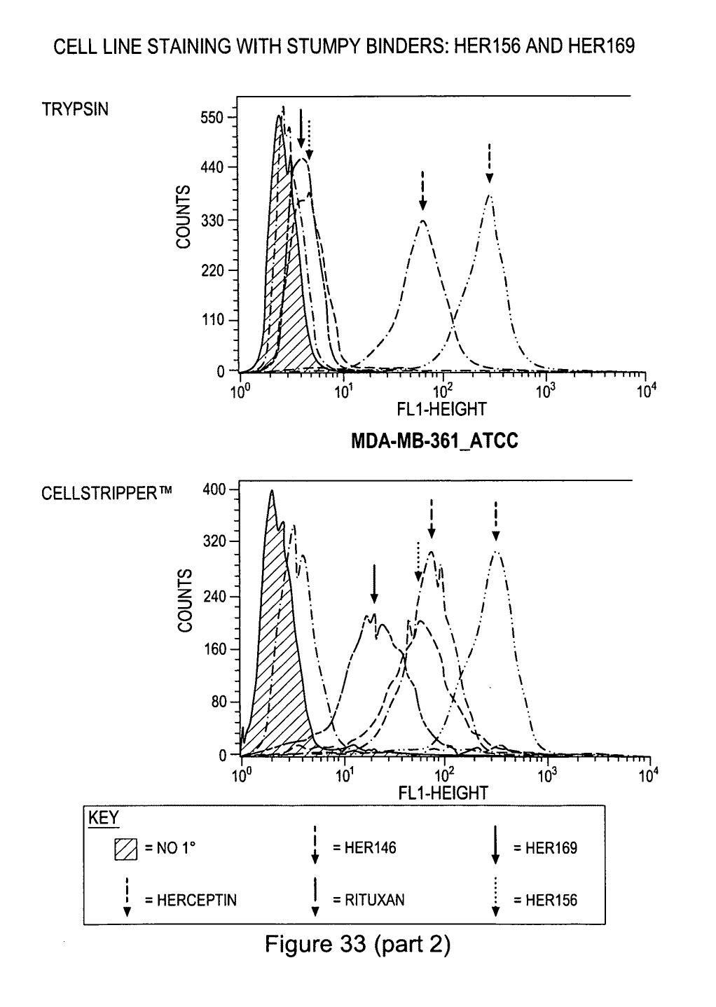

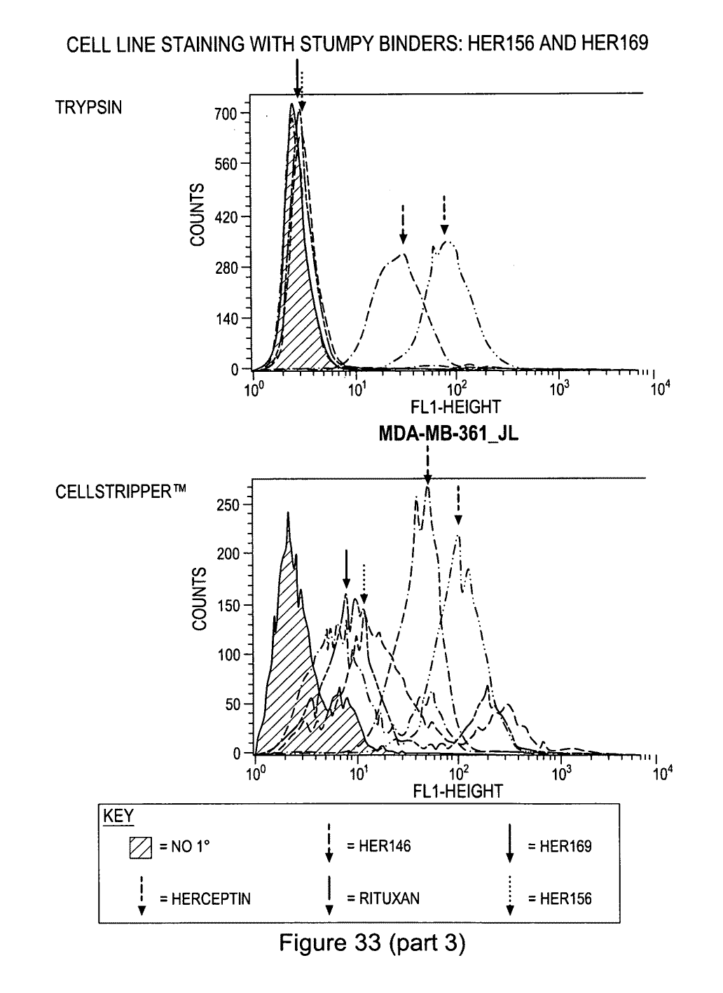

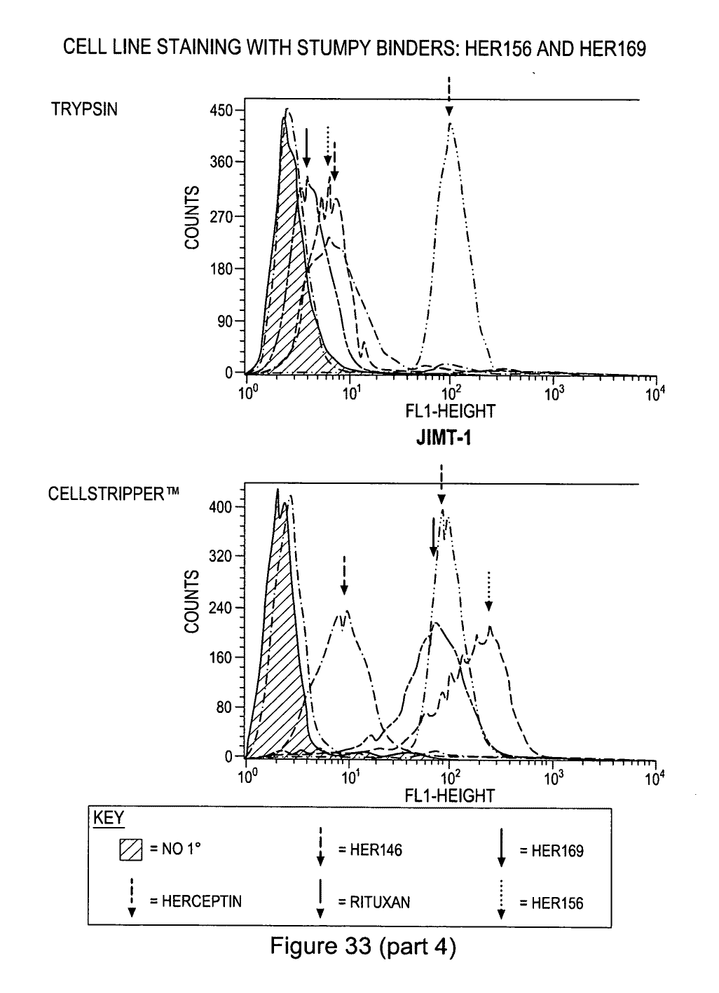

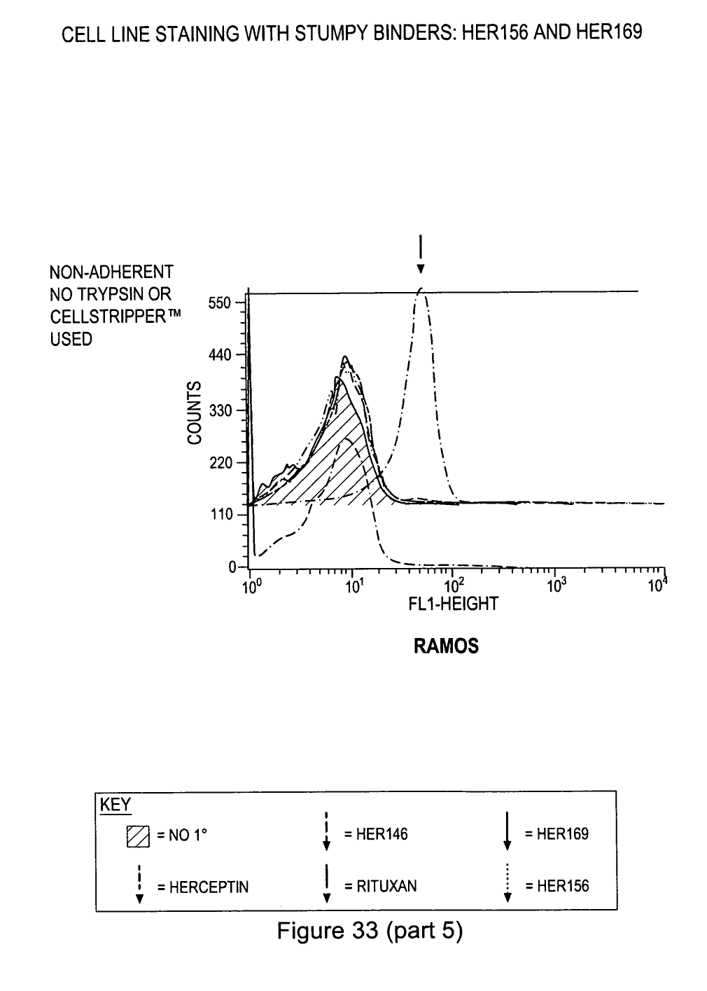

We further investigated the effects of enzymatic vs non-enzymatic preparation of the cells on SMIP binding. Briefly, we removed media from cells and rinsed them with PBS (−Ca/Mg). We then dissociated the cells with trypsin (0.25%) or non-enzymatic CellStripper™ (Cellgro Mediatech #25-056-C1). We washed dissociated cells once in cold media (containing FBS), and washed them again in cold PBS (+Ca/Mg). We then resuspended the cells in cold Staining Buffer (PBS, +Ca/Mg, 2% FBS) and counted resuspended cells. We stained 300,000-350,000 cells per well on ice in 100 μl Staining Buffer +/− primary at 200 nM SMIP/Antibody (200 nM=22 μg/ml SMIP) with 1 μg/ml PI. We then washed the stained cells three times with cold Staining Buffer. We performed secondary staining with 5 μg/ml Alexa Fluor 488-conjugated Goat anti-Human IgG (Molecular Probes #A-11013) with 1 μg/ml PI. We then fixed the cells in 1% Paraformaldehyde in Staining Buffer and analyzed the next day. Results of various cell line staining with “stumpy binders” HER 156 and HER169 are shown in FIG. 33.

Species Cross-Reactivity

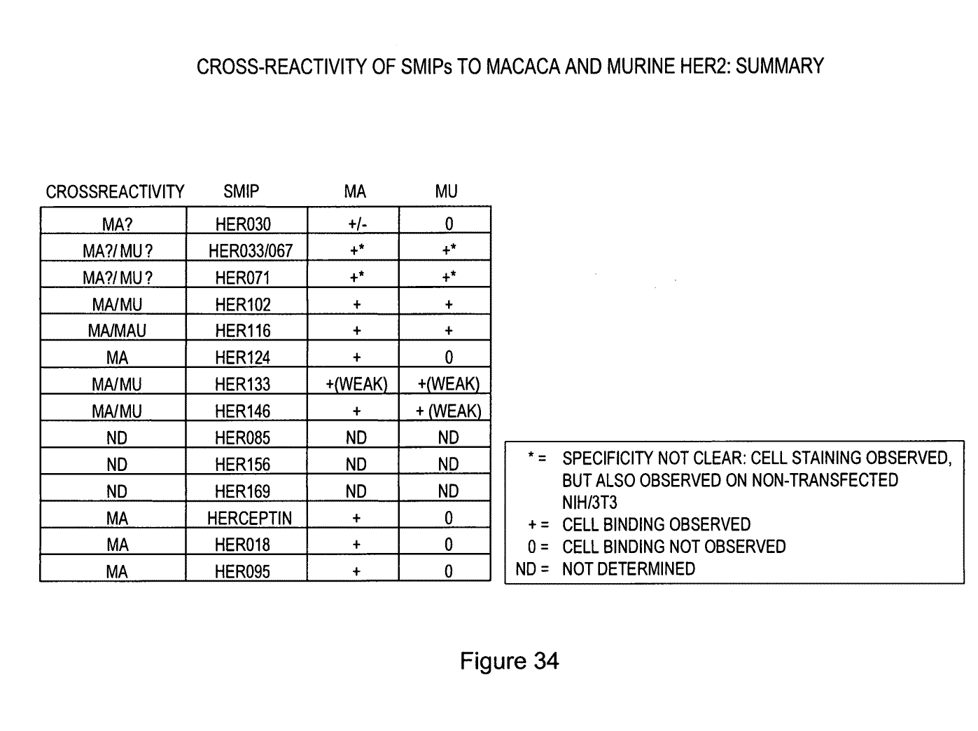

To determine species cross-reactivity, we performed the above-mentioned binding assay with NIH/3T3 cells transfected with Macaca Her2 and with CHO cells expressing Murine Her2. The results of the cross-reactivity binding studies are summarized in FIG. 34. Herceptin®, HER018, HER095, and HER124 cross-react with Macaca Her2, while HER102, HER116, HER133, and HER146 cross-react with both Macaca Her2 and Murine Her2.

### Example 5

### PCR Amplification of scFv Regions for Sequencing Analysis

PCR amplification of scFvs was carried out using the KOD HOT START DNA Polymerase kit (Novagen) in accordance with the manufacturers instructions. 0.2 μM each of the M13rev (5′ GGAAACAGCTATGACCATGA 3′) (SEQ ID NO: 247) forward and Mycseq (5′ CTCTTCTGAGATGAGTTTTTG 3′) (SEQ ID NO: 248) reverse primers were used. 5 μl of a 1:10 dilution of a stationary phase bacterial culture was used as the template for a final reaction volume of 20 μl. The cycling conditions used were a 2 minute hot start at 94° C., 25 cycles of denaturation at 94° C. (1 minute), primer annealing at 42° C. (30 seconds) and extension at 72° C. (1 min), followed by a final 5 minute extension at 72° C. PCR products were verified by agarose gel electrophoresis and cleaned up with Exol/SAP (shrimp alkaline phosphatase) prior to sequencing of both strands with primers 145837 (5′ GGAGATTTTCAACGTGAA 3′) (SEQ ID NO: 249) and 142051 (5′ CTCTTCTGAGATGAGTTTTTG 3′) (SEQ ID NO: 250). The closest human germlines of the V_{H} and V_{L} segments were determined (Table 4).

TABLE 4
V_{H} and V_{L} germlines of ERBB2 clones

| Mab | Human V_{H} germline gene | Human V_{L} germline gene |
|---|---|---|
| S1R2A_CS_1F7 | 1-02 | Vλ 3h |
| | (DP8/75) | |
| S1R2A_CS_1D11 | 1-69 | Vλ 1b (DPL5) |
| | (DP10) | |
| S1R2C_CS_1D3 | 1-69 | Vλ 1b (DPL5) |
| | (DP10) | |
| S1R2C_CS_1H12 | 3-48 | Vλ 1c (DPL2) |
| | (DP51) | |
| S1R2A_CS_1D3 | 1-02 | Vλ 1g (DPL3) |
| | (DP8/75) | |
| S1R3B2_BMV_1E1 | 3-33 | Vλ 1b (DPL5) |
| | (DP50) | |
| S1R3C1_CS_1D3 | 6-1 | Vλ 2c |
| | (DP74) | |
| S1R3B2_DP47_1E8 | 3-23 | Vλ 1e (DPL8) |
| | (DP47) | |
| S1R3B2_BMV_1G2 | 1-18 | Vκ L12 |
| | (DP14) | |
| S1R3B2_BMV_1H5 | 3-33 | Vλ 2a2 (DPL11) |
| | (DP50) | |
| S1R3C1_CS_1A6 | 5-51 | Vλ 1c (DPL2) |
| | (DP73) | |
| S1R3B2_DP47_1C9 | 3-23 | Vλ 1c (DPL2) |
| | (DP47) | |
| S1R3B2_DP47_1E10 | 3-23 | Vλ 1g (DPL3) |
| | (DP47) | |
| S1R3C1_CS_1B10 | 1-69 | Vλ 6a |
| | (DP10) | |
| S1R3A1_BMV_1F3 | 3-21 | Vλ 31 (DPL16) |
| | (DP77) | |
| S1R3B1_BMV_1G11 | 3-23 | Vλ 2a2 (DPL11) |
| | (DP47) | |
| S1R3A1_BMV_1G4 | 1-03 | Vλ 2a2 (DPL11) |
| | (DP25) | |
| S1R3B1_BMV_1H11 | 3-23 | Vκ L12 |
| | (DP47) | |
| S1R3A1_CS_1B9 | 5-51 | Vλ 8a (DPL21) |
| | (DP73) | |
| S1R3B1_BMV_1H9 | 4-04 | Vλ 3l (DPL16) |
| | (DP70) | |
| S1R3A1_CS_1B10 | 1-02 | Vλ 8a (DPL21) |
| | (DP8/75) | |
| S1R3B1_BMV_1C12 | 3-30.5 | Vλ 1c (DPL2) |
| | (DP49) | |
| S1R3C1_BMV_1H11 | 3-33 | Vλ 1e (DPL8) |
| | (DP50) | |
| S1R3B1_BMV_1A10 | 3-30.5 | Vλ 3l (DPL16) |
| | (DP49) | |
| S1R3A1_CS_1D11 | 5-51 | Vλ 8a (DPL21) |
| | (DP73) | |
| S1R3C1_DP47_1H1 | 3-23 | Vλ 3h |
| | (DP47) | |
| S1R3A1_CS_1B12 | 1-02 | Vλ 1e (DPL8) |
| | (DP8/75) | |
| S1R3B1_BMV_1H5 | 3-33 | Vλ 3l (DPL16) |
| | (DP50) | |
| S1R3A1_DP47_1A6 | 3-23 | Vλ 1c (DPL2) |
| | (DP47) | |
| S1R3B1_DP47_1E1 | 3-23 | Vλ 6a |
| | (DP47) | |
| S1R3B1_BMV_1A1 | 1-18 | Vλ 2a2 (DPL11) |
| | (DP14) | |
| S1R3B1_DP47_3A2 | (3-23) | Vlambda2_DPL11_(2a2) |
| | Vh3_DP-47_{—} | |
| S1R3A1_DP47_11B7 | (3-23) | Vlambda1_DPL8_(1e) |
| | Vh3_DP-47_{—} | |
| S1R3A1_DP47_11D1 | (3-23) | Vlambda3_DPL16_(3l) |
| | Vh3_DP-47_{—} | |
| S1R3A1_DP47_7F3 | (3-23) | Vlambda1_DPL8_(1e) |
| | Vh3_DP-47_{—} | |
| S1R2B_DP47_4E3 | (3-23) | Vlambda1_DPL3_(1g) |
| | Vh3_DP-47_{—} | |
| S1R3C1_DP47_2G2 | (3-23) | Vlambda1_DPL2_(1c) |
| | Vh3_DP-47_{—} | |
| S1R3A1_DP47_11H6 | (3-23) | Vlambda1_DPL3_(1g) |
| | Vh3_DP-47_{—} | |
| S1R3A1_BMV_3B1 | (3-30.5) | Vlambda2_DPL11_(2a2) |
| | Vh3_DP-49_{—} | |
| S1R3A1_DP47_6B9 | (3-23) | Vlambda1_DPL8_(1e) |
| | Vh3_DP-47_{—} | |
| S1R2A_CS_10B8 | (1-46) | Vlambda1_DPL2_(1c) |
| | Vh1_DP-7_{—} | |
| S1R3A1_DP47_7A6 | (3-23) | Vlambda1_DPL8_(1e) |
| | Vh3_DP-47_{—} | |
| S1R3B2_DP47_2G3 | (3-23) | Vlambda3_DPL16_(3l) |
| | Vh3_DP-47_{—} | |
| S1R2B_CS_6H11 | (5-51) | Vlambda8_DPL21_(8a) |
| | Vh5_DP-73_{—} | |
| S1R3A1_DP47_10G1 | (3-23) | Vlambda1_DPL8_(1e) |
| | Vh3_DP-47_{—} | |
| S1R3A1_DP47_7C1 | (3-23) | Vlambda1_DPL8_(1e) |
| | Vh3_DP-47_{—} | |
| S1R2A_DP47_5D6 | (3-23) | Vk3_DPK22_(A27) |
| | Vh3_DP-47 | |
| S1R3A1_DP47_11F6 | (3-23) | Vlambda1_DPL5_(1b) |
| | Vh3_DP-47_{—} | |
| S1R3A1_DP47_11D3 | (3-23) | Vlambda1_DPL3_(1g) |
| | Vh3_DP-47_{—} | |
| S1R3A1_CS_8A8 | (5-51) | Vlambda8_DPL21_(8a) |
| | Vh5_DP-73_{—} | |
| S1R3A1_BMV_5D10 | (3-23) | Vk1_L12 |
| | Vh3_DP-47_{—} | |
| S1R3A1_DP47_11C1 | (3-23) | Vlambda3_DPL16_(3l) |
| | Vh3_DP-47_{—} | |
| S1R3A1_DP47_4E1 | (3-23) | Vlambda1_DPL8_(1e) |
| | Vh3_DP-47_{—} | |
| S1R3A1_DP47_10E1 | (3-23) | Vlambda1_DPL2_(1c) |
| | Vh3_DP-47_{—} | |
| S1R3A1_CS_11C3 | (1-18) | Vlambda3_DPL16_(3l) |
| | Vh1_DP-14_{—} | |
| S1R3A1_CS_13H11 | (1-02) | Vlambda1_DPL5_(1b) |
| | Vh1_DP-8,75_{—} | |
| S1R3A1_CS_2D9 | (5-51) | Vk1_L12 |
| | Vh5_DP-73_{—} | |
| S1R2A_CS_3D4 | (1-02) | Vlambda1_DPL8_(1e) |
| | Vh1_DP-8,75 | |
| S1R3A1_DP47_2H6 | (3-23) | Vlambda3_DPL23_(3r) |
| | Vh3_DP-47_{—} | |
| S1R3A1_DP47_4G1 | (3-23) | Vlambda3_3h |
| | Vh3_DP-47_{—} | |
| S1R2A_DP47_3C1 | (3-23) | Vlambda3_DPL23_(3r) |
| | Vh3_DP-47_{—} | |
| S1R3A1_DP47_7B2 | (3-23) | Vk1_DPK6_(L19) |
| | Vh3_DP-47_{—} | |
| S1R3B2_DP47_4E2 | (3-23) | Vlambda1_DPL2_(1c) |
| | Vh3_DP-47_{—} | |
| S1R3A1_CS_16C2 | (3-23) | Vlambda3_DPL16_(3l) |
| | Vh3_DP-47_{—} | |
| S1R3A1_CS_11E5 | (5-51) | Vlambda8_DPL21_(8a) |
| | Vh5_DP-73_{—} | |
| S1R3A1_CS_16D7 | (5-51) | Vlambda8_DPL21_(8a) |
| | Vh5_DP-73_{—} | |
| S1R2A_CS_10B10 | (1-02) | Vlambda8_DPL21_(8a) |
| | Vh1_DP-8,75_{—} | |
| S1R3A1_CS_15C2 | Vh4_DP-70_(4-04) | Vlambda3_DPL16_(3l) |
| S1R3A1_CS_9C1 | Vh4_DP-67_(4-b) | Vlambda1_DPL8_(1e) |
| S1R2A_CS_5A1 | Vh3_DP-47_(3-23) | Vlambda1_DPL2_(1c) |
| S1R2A_CS_8C8 | Vh5_DP-73_(5-51) | Vlambda8_DPL21_(8a) |
| S1R3A1_CS_13H5 | Vh5_DP-73_(5-51) | Vlambda8_DPL21_(8a) |
| S1R2B_CS_5E9 | Vh5_DP-73_(5-51) | Vlambda8_DPL21_(8a) |
| S1R3A1_CS_8F9 | Vh5_DP-73_(5-51) | Vlambda8_DPL21_(8a) |
| S1R3A1_CS_14B5 | Vh5_DP-73_(5-51) | Vlambda8_DPL21_(8a) |
| S1R2A_CS_9E10 | Vh5_DP-73_(5-51) | Vlambda8_DPL21_(8a) |
| S1R3A1_CS_7A10 | Vh5_DP-73_(5-51) | Vlambda8_DPL21_(8a) |
| S1R3A1_BMV_6H7 | Vh3_DP-46_(3- | Vk1_L12 |
| | 30.3) | |
| S1R3A1_CS_12A11 | Vh5_DP-73_(5-51) | Vlambda8_DPL21_(8a) |
| S1R3A1_CS_13D12 | Vh5_DP-73_(5-51) | Vlambda8_DPL21_(8a) |
| S1R3A1_CS_7A8 | Vh4_DP-79_(4-39) | Vlambda8_DPL21_(8a) |
| S1R2A_CS_2C9 | Vh1_DP-7_(1-46) | Vk3_DPK21_(L2) |
| S1R3A1_CS_12D1 | Vh3_3-73 | Vlambda1_DPL8_(1e) |
| S1R2A_CS_7D4 | Vh1_DP-5_(1-24) | Vlambda8_DPL21_(8a) |
| S1R3A1_CS_15B8 | Vh5_DP-73_(5-51) | Vlambda8_DPL21_(8a) |
| S6R3_DP47_1A10 | 3-23 | Vλ 1c (DPL2) |
| | (DP47) | |
| S6R2_DP47_1E11 | 3-23 | Vλ 1c (DPL2) |
| | (DP47) | |
| S5R2_DP47_1H11 | 3-23 | Vλ 1c (DPL2) |
| | (DP47) | |
| S6R3_CS_1G5 | 4-39 | Vλ 1g (DPL3) |
| | (DP79) | |
| S6R2_DP47_1H11 | 3-23 | Vλ 1b (DPL5) |
| | (DP47) | |
| S5R3_DP47_1A10 | 3-23 | Vλ 1e (DPL8) |
| | (DP47) | |
| S5R2_DP47_1D11 | 3-23 | Vλ 1e (DPL8) |
| | (DP47) | |
| S5R2_CS_1A8 | 5-51 | Vλ 1e (DPL8) |
| | (DP73) | |
| S6R3_CS_1B7 | 5-51 | Vλ 1e (DPL8) |
| | (DP73) | |
| S6R2_CS_1E5 | 1-02 | Vλ 2e2 (DPL11) |
| | (DP-8,75) | |
| S6R3_BMV_1C2 | 7-4.1 | Vλ 2a2 (DPL11) |
| | (DP21) | |
| S5R2_DP47_1B10 | 3-23 | Vλ 3l (DPL16) |
| | (DP47) | |
| S6R3_DP47_1C12 | 3-23 | Vλ 3l (DPL16) |
| | (DP47) | |
| S5R2_DP47_1D10 | 3-23 | Vλ 3l (DPL16) |
| | (DP47) | |
| S6R3_DP47_1H9 | 3-23 | Vλ 3l (DPL16) |
| | (DP47) | |

### Example 6

### BIACORE® Binding Assays

Binding of different Her2-directed binders (antibodies and SMIPs) to monomeric Her2 ECD and truncations of dimeric Her2 ECD were determined using a BIACORE® T100 instrument (GE Healthcare, Biacore, Piscataway, N.J.). We conducted the binding experiments in both orientations, i.e., first using anti-HER2SMIPS as ligands and then as analytes.

SMIPs as Ligands

Her2-directed binders were captured on a chip by a monoclonal mouse anti-human Fc (GE healthcare), which was covalently conjugated to a carboxylmethyl dextran surface (CM4) via amines using N-ethyl-N′-(3-dimethylaminopropyl)-carbodiimide hydrochloride and N-hydroxysuccinimide. The unoccupied sites of the activated surface were blocked by ethanolamine. The capturing antibody (referred to as anti hFc) binds to the C_{H}2 domain of IgG Fc of all sub-classes and showed no discernible dissociation from the captured her2-binders during the course of the assay. Every cycle, 3 different Her2 binders and a non-binder (negative control) were individually captured by anti hFc on 4 different flow cells, typically to about 50 RU, followed by injection of the analyte (Her2 dimers and monomer) at a particular concentration for 10 minutes over all flow cells. The dissociation of the formed complexes were subsequently followed for 12 minutes. At the end of the cycle, the surface was regenerated gently using 3M MgCl₂ which dissociates protein bound to the capturing anti hFc antibody. Multiple such cycles were performed to study binding of different analytes at different concentrations, in the range of 0-300 nM, for each set of three Her2 binders captured. Her2 binders were reproducibly captured every cycle with CV not exceeding 1%. The binding was performed at 25° C. in 0.01 M HEPES pH 7.4, 0.15 M NaCl, 0.005% v/v SURFACTANT P20. Signal associated with binding to the negative control was used to subtract for bulk refractive changes. The kinetic parameters and affinities were determined using BIAEVALUATION software.

SMIPS as Analytes

In these experiments, the trastuzumab (HERCEPTIN®) and anti-HER2 SMIPs were used as the analytes and the soluble HER2 receptors were used as the ligands. In one experiment, SMIPs and trastuzumab were flowed over a histidine-tagged monomeric HER2 receptor that was bound to a Ni²⁺-nitrilotriacetic acid surface. In a second experiment, SMIPs and trastuzumab were flowed over a histidine-tagged HER2 receptor that was captured by an anti-6-histidine-tagged monoclonal antibody conjugated to a CM4 surface. In a third experiment, SMIPs and trastuzumab were flowed over a HER2 receptor that was directly amine-coupled to a CM4 surface. The binding in each of these three experimental designs was performed at 25° C. When employing any of the three experimental designs listed above, similar results were obtained. Signal associated with binding to the negative control was used to subtract for bulk refractive changes. The kinetic parameters and affinities were determined using BIAEVALUATION software.

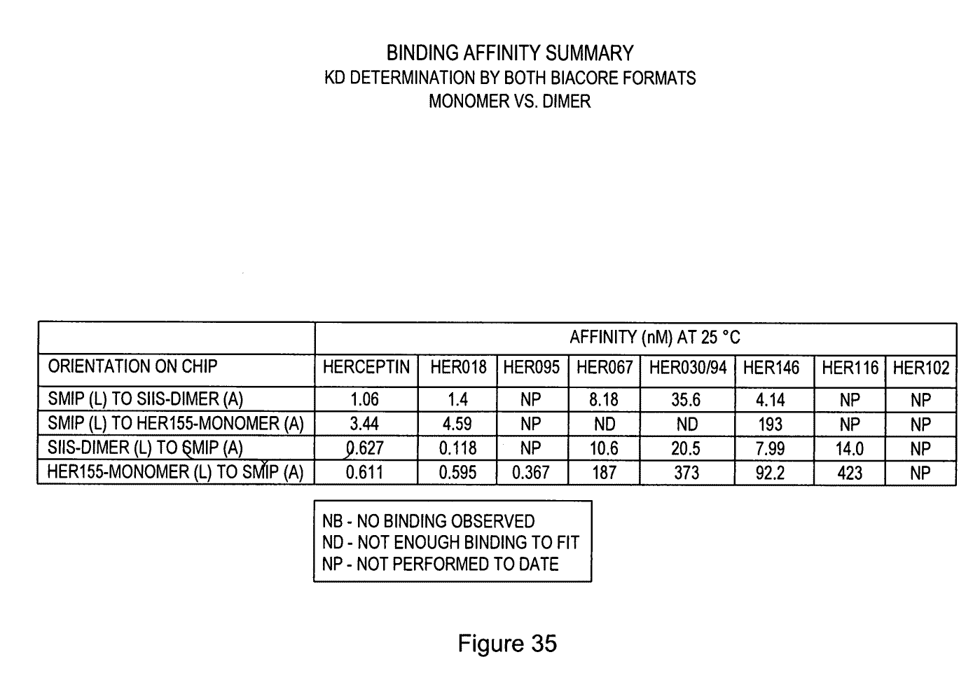

The results of the BIACORE® assays are shown in FIGS. 7 and 35 and in Table 5. HERCEPTIN® (trastuzumab) and the HERCEPTIN® SMIP bound monomeric and dimeric HER2 receptors similarly in both orientations of the BIACORE® assay. HER095 bound monomeric HER2 at sub-nanomolar affinity. In contrast, the HER067, HER033, HER030/HER094, HER 146, HER116 and HER102SMIPS bound more strongly to dimeric soluble HER2 recpetor than to monomeric HER2 receptor. The HER033 and HER067 SMIPs have the same amino acid sequence, but the difference between them is that the former is produced in HEK cells while the latter is produced in CHO cells. Binding by HER033 and HER067SMIPs is substantially the same. HER030 appears to bind less strongly than Her033/Her067 to the dimers. Specificity for dimeric HER2 may be advantageous in that such binders may have increased selectivity for tumors and may not bind, or show reduced binding to tissues that express low levels of HER2 and/or where ligand independent homodimer formation is limited. Such HER2 binders with reduced binding to non-tumor target tissues (e.g., cardiac tissues) may, thus, have fewer side effects including lower toxicity. In addition, a lack of binding to shed HER2 ectodomain would reduce the effective dose compared to a HER2-binding agent that has significant binding to shed ECD.

TABLE 5
BIACORE ® Affinity Data

| | Affinity (nM) at 25° C. | | | | | |
|---|---|---|---|---|---|---|
| | Herceptin | Her 018 | Her 033 | Her 067 | Her 030 | Her 146 |
| SIIS (Dimer) | 1.06 | 1.4 | 7.23 | 8.18 | 35.6 | 4.14 |
| 1.8 (Dimer) | 228 | 167 | 4.92 | 6.47 | 27.6 | 2.8 |
| 1.6 (Dimer) | NB | NB | NB | NB | NB | 4.2 |
| Her155 | 3.44 | 4.59 | 508 | ND | ND | 193 |
| (Monomer) | | | | | | |

NB—No Binding Observed
ND - not enough binding to fit

### Example 7

### BrdU and ATP Proliferation Assays

To 96-well plates, cells were added at 2.5×10³ cells/well (SKBR3, BT474, MDA-MB-453, MDA-MB-175) or at 5×10³ cells/well (MDA-MB-361). The next day, SMIPs were added to the cells at the desired concentration and then incubated at 37° C./5% CO₂ for 4 (SKBR3, MDA-MB-453, MDA-MB-361, MDA-MB-175), 5 (BT474), or 7 (MDA-MB-361) days. The day before cells were harvested, 5-bromo-2′-deoxyuridine (BrdU) is added to a final concentration of 0.1 mM and continued to incubate overnight at 37° C. After incubation, media was removed and then the cells were treated with ethanol-based fix solution (DELFIA® Cell Proliferation Kit, Perkin Elmer, Waltham, Mass.) at room temperature (RT) for 30 minutes. Fix solution was removed by aspiration, 100 μl/well anti-BrdU-Eu labeled antibody (0.5 mg/mL) was added, and the cells were incubated at RT for 2 hours. Cells were then washed 4 times with Tris-based DELFIA Platewash (300 μl/well/wash). DELFIA Inducer (with Triton® X-100, glycine, HCl, and chelator) was then added to the cells (200 μl/well) and incubated with shaking for 15 minutes at RT. Fluorescence was measured using Flex Station® 3 in Time resolved fluorescence mode (Molecular Devices, Sunnyvale, Calif.).

After the proliferation assay fluorescence reading, the DELFIA Inducer was removed by aspiration and Hoechst 33342 nuclear stain solution (Invitrogen, Carlsbad, Calif.) was added to the cells. Nuclear stain fluorescence was measured on an IN Cell Analyzer at 4× resolution.

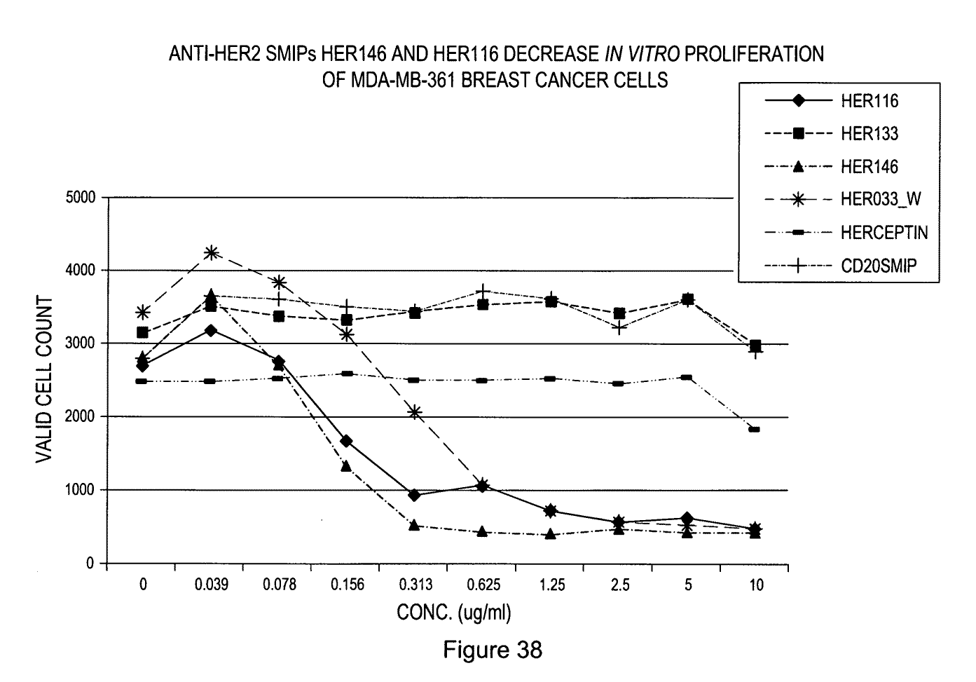

Alternatively, we investigated anti-Her2 SMIP anti-proliferation activity in MDA-MB-361 cells as follows. MDA-MB-361 breast cancer cells were plated in 96-well format and treated with anti-Her2 or control reagents for indicated concentrations and times (24-96 hr). For proliferation assays, media (DMEM plus 10% FBS) was removed, the cells washed with phosphate-buffered saline (PBS), fixed with 4% paraformaldehyde and nuclei stained with DAPI (Molecular Probes). Stained nuclei were counted using Cellomics High Content assay measuring fluorescence at 360 nM. The results are shown in FIG. 38. For apoptosis assay, fixed cells were permeabilized by treatment with 0.2% Triton 100 in PBS prior to primary staining with mouse anti-cleaved PARP antibody (Cell Signaling Technologies) and secondary staining with goat anti-mouse IgG labeled with ALEXA488 (Invitrogen). Fluorescence was measured in Cellomics High Content assay at 488 nM.

ATP Lite First Step assay (Perkin Elmer) was used to assess cellular viability by measuring ATP levels via luminescence (ATP luciferase). To 96-well plates, cells were added at 2.5×10³ cells/well (SKBR3, BT474, MDA-MB-453, MDA-MB-175) or at 5×10³ cells/well (MDA-MB-361). The next day, SMIPs were added to the cells at the desired concentration and then incubated at 37° C./5% CO₂ for 4 (SKBR3, MDA-MB-453, MDA-MB-361, MDA-MB-175), 5 (BT474), or 7 (MDA-MB-361) days. After SMIP incubation for the desired amount of time, lyophilized ATP Lite substrate is reconstituted with 10 ml of ATP Lite substrate/lysis solution and allowed to sit at room temperature for 10 minutes. This reconstituted substrate solution was added to the cells (100 μl/well) and read luminescence on Top Count Reader (Packard).

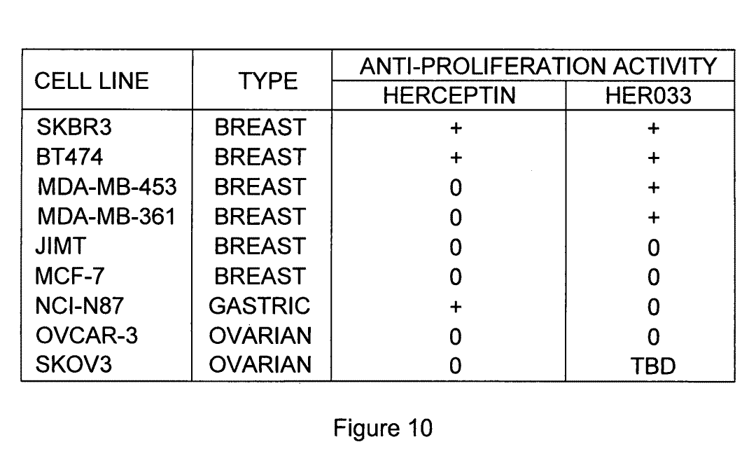

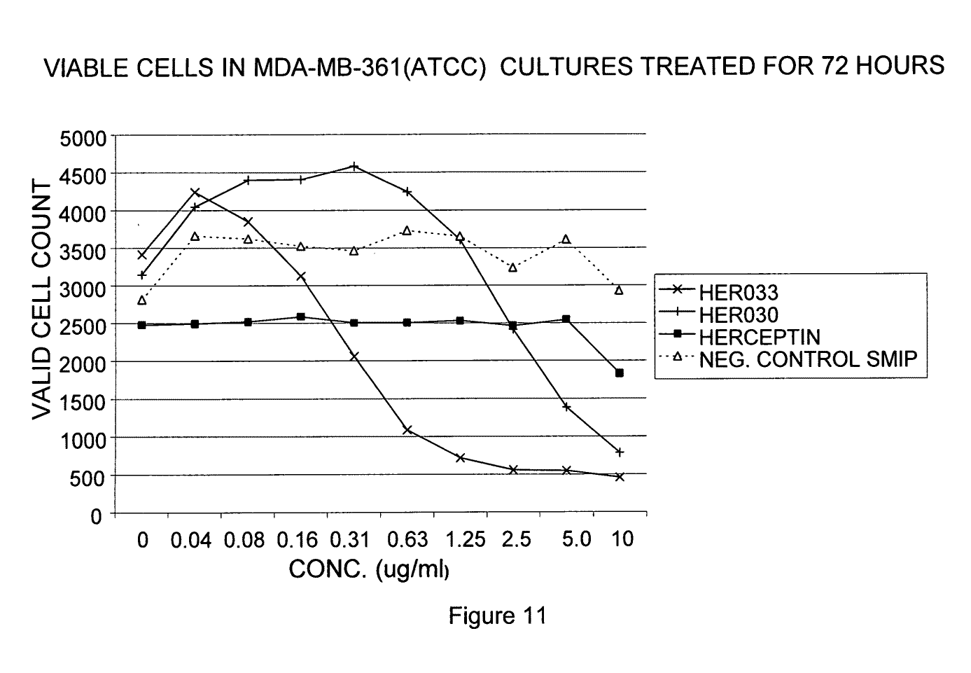

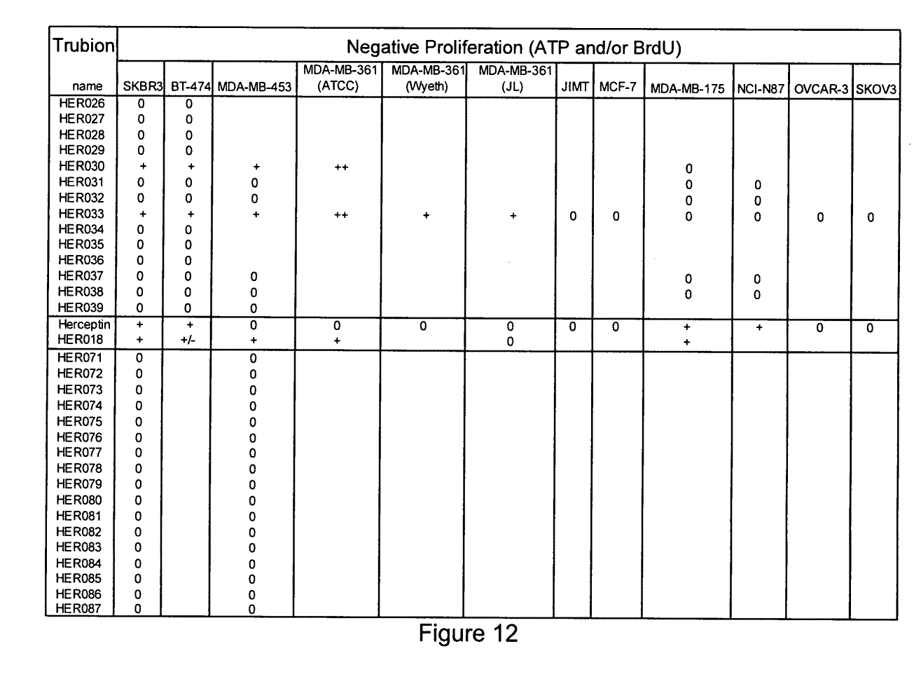

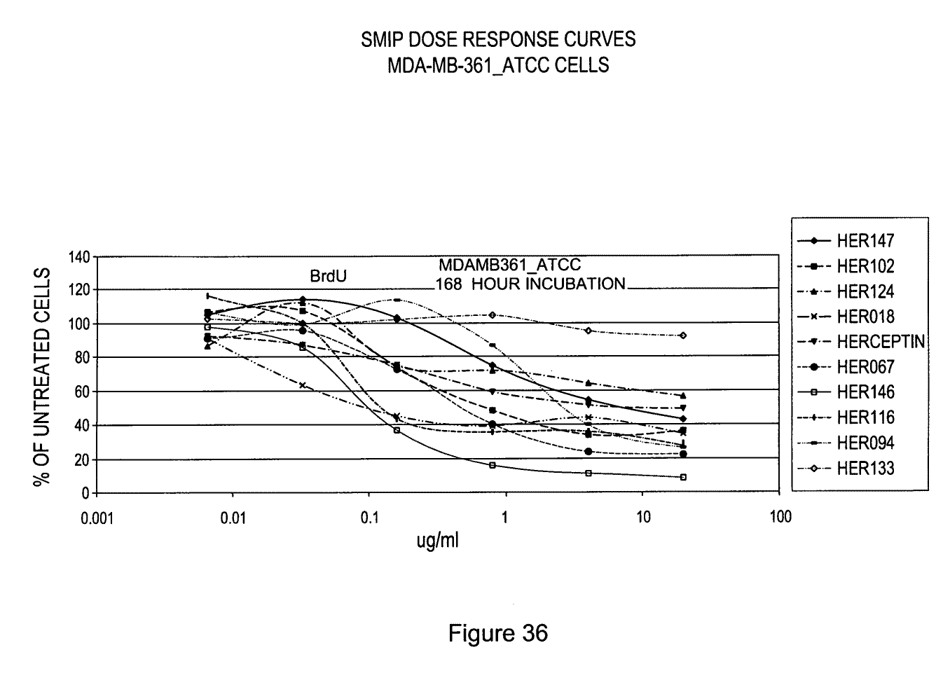

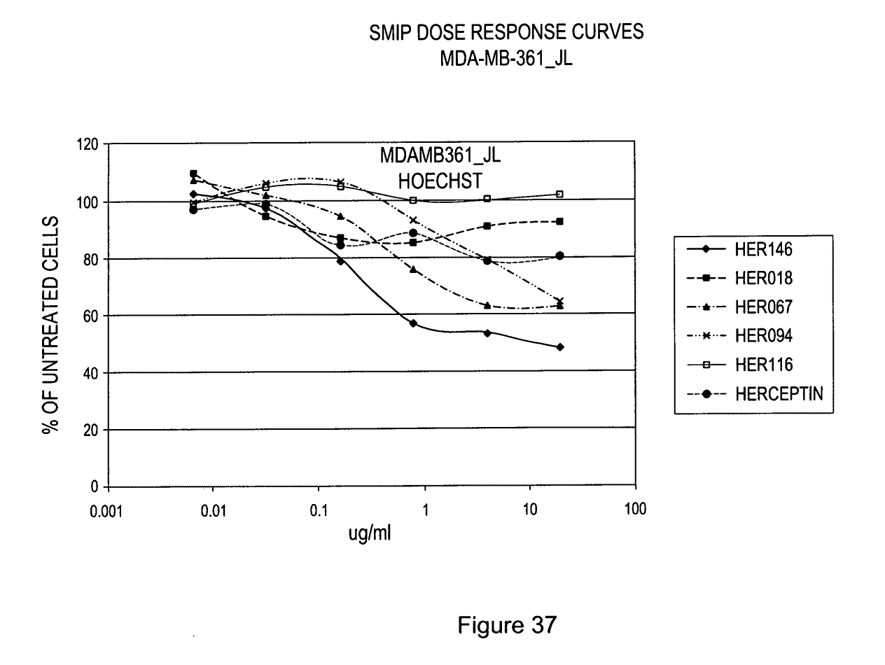

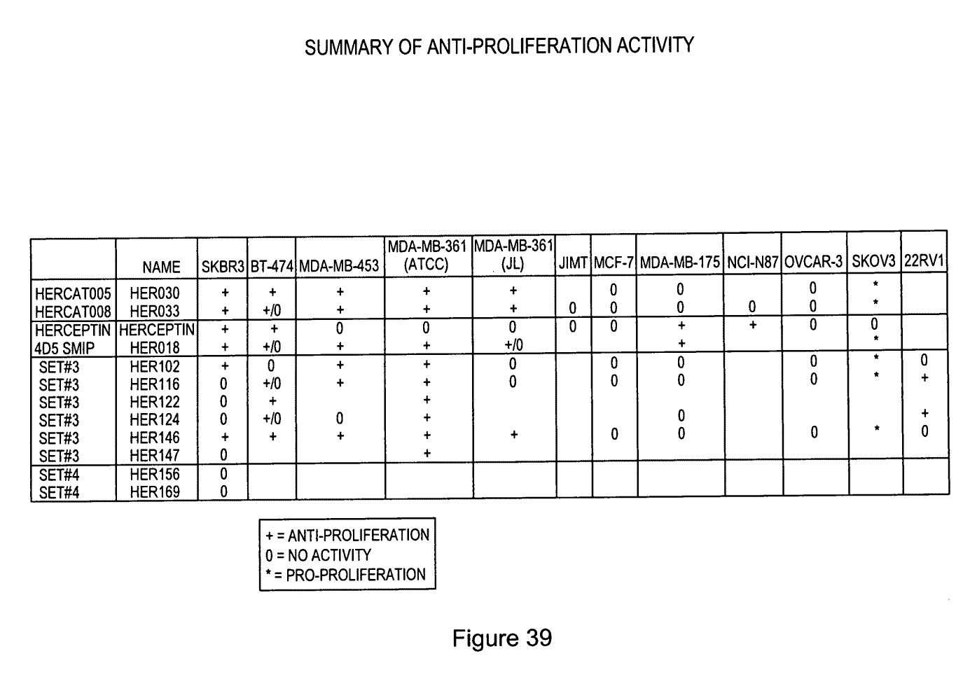

The results of the proliferation assays are shown in FIGS. 10-12 and FIGS. 36-38 and are summarized in FIG. 39. As shown in the Figures, the anti-HER2 SMIPS represent different groups of HER2 binders that bind different domains of HER2 and having differential ability to decrease proliferation in multiple cell lines. As shown, anti-HER2 SMIPS reduce proliferation of a different repertoire of cell lines than HERCEPTIN®, the SMIP form of HERCEPTIN® has a different repertoire of cell killing than the parent antibody and HER2SMIPS differ from each other in the cell lines in which they reduce proliferation.

### Example 8

### Pathway Phosphorylation Assays

To 96-well plates, cells were added at 8−12×10³ cells/well depending on cell type (Becton-Dickinson, San Jose, Calif.) and allowed to incubate overnight in growth medium with serum at 37° C./5% CO₂. After removal of growth medium, the cells were washed with serum-free medium, aspirated, and then serum-free media was added for incubation at 37° C./5% CO₂ for 3 hours. The SMIP of interest was prepared in prewarmed serum-free media, added to each well at the indicated concentration, and incubated at 37° C./5% CO₂ for desired time points. As a control, signaling was inhibited with AG825 (Calbiochem, LaJolla, Calif.) at 40 μM; LY294002 (Cell Signaling) at 50 μM; or U0126 MEK1/2 inhibitor (Cell Signaling) at 10 μM. The cells were then fixed in formaldehyde (diluted in 1×PBS) at a final concentration of 3.7% for 10 minutes at 37° C./5% CO₂. The cells were then washed two times with PBS. After removing the PBS, the cells were permeabilized in 0.1% Triton® X-100 (Sigma-Aldrich, St. Louis, Mo.) solution diluted in 1×PBS at room temperature for 5 minutes. The cells were then washed two times with PBS and blocked by incubation in PBS/1% BSA (Sigma-Aldrich) at room temperature for 30 minutes (or overnight at 4° C.).

The blocking solution was removed and primary antibody (in PBS with 3% horse serum or PBS with 1% BSA, and 0.1% Triton® X-100) was added for 1 hour at room temperature (or overnight at 4° C.). The primary antibodies used (at 0.125 μg/well) were (1) rabbit anti-phospho-akt (Ser473) (Cell Signaling, Danvers, Mass.); (2) mouse anti-phospho-Erk1/2 (Cell Signaling, Danvers, Mass.); and (3) rabbit anti-phospho-ErbB2 (Abgent, San Diego, Calif.). The primary antibody was removed and the cells were washed 3 times with PBS. The secondary antibody (in PBS with 3% horse serum or PBS with 1% BSA, and 0.1% Triton® X-100) was then added for 1 hour at room temperature (or overnight at 4° C.) protected from light. The secondary antibodies used (at 0.2 μg/well) were Alexa 488 donkey anti-rabbit IgG (Invitrogen, Carlsbad, Calif.) and DyLight 649 goat anti-ms IgG (Pierce, Rockford, Ill.). The secondary antibody was removed and the cells were washed 3 times with PBS. Then 100 μL of PBS containing 200 ng/ml Hoechst 33342 nuclear stain (Invitrogen, H3570) (and if needed 1 ug/ml CellMask Blue cytoplasmic stain (Invitrogen, H34558) was added to the cells. The plates were covered and kept protected from light. The plates were then imaged.

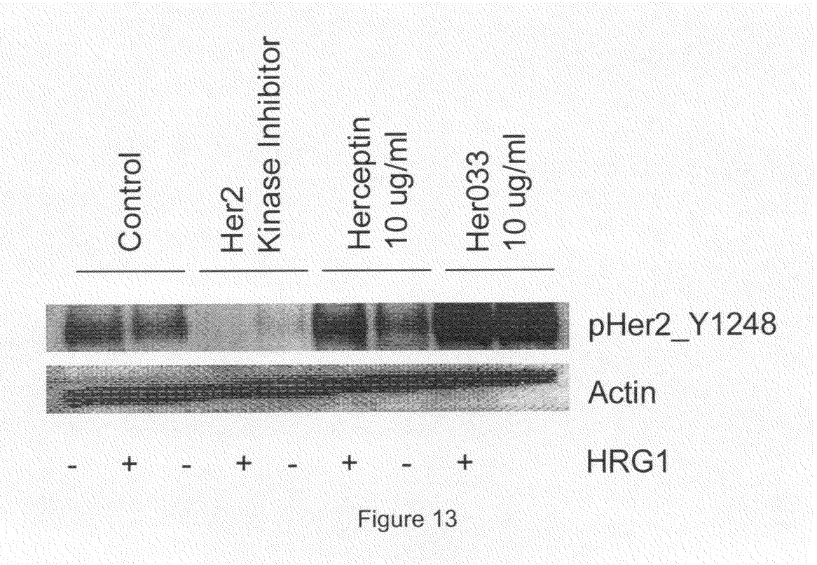

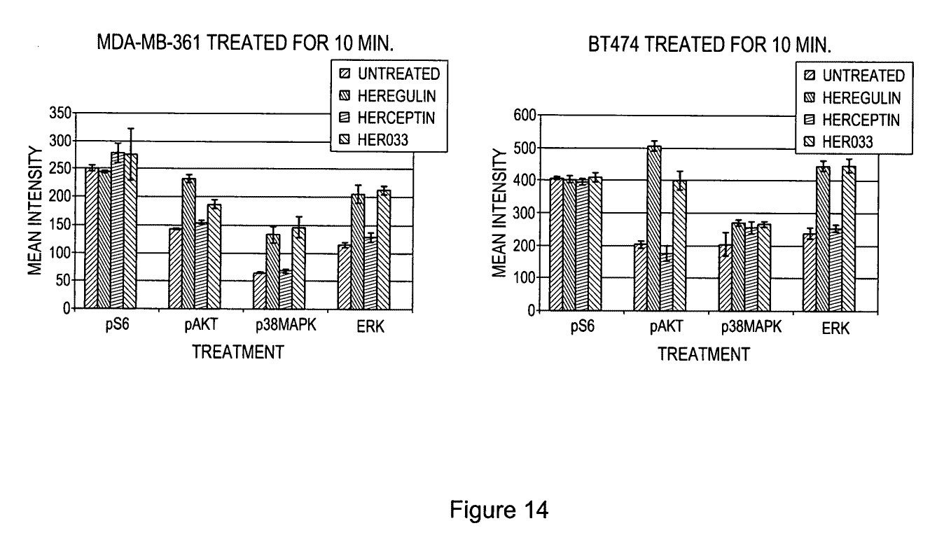

Alternatively, we investigated anti-Her2 SMIP signal transduction activity in MDA-MB-361 cells as follows. MDA-MB-361 breast cancer cells, were plated in 6-well plate to 80-90% confluency (DMEM plus 10% FBS) and treated with anti-Her2 or control reagents for 24 hr with and without pretreatment with Heregulin (HRG−15 min.) or EGF (30 min.). For assay of total and phosphorylated Her2, cells were lysed, 50 ug total protein was fractionated using SDS-PAGE and transferred to nitrocellulose membranes using standard procedures. Western blot analysis used either rabbit anti-Her2 antibody (Cell Signaling Technologies), anti-pHer2_Y1248 (Upstate) or anti-Actin (Santa Cruz) as primary antibody and subsequently stained with HRP-conjugated anti-rabbit IgG. Peroxidase activity was measured using ECLplus2 kit (GE Healthcare) following manufacturer's protocols and exposed to film. As shown in FIG. 13, HER033 induces HER2 phosphorylation.

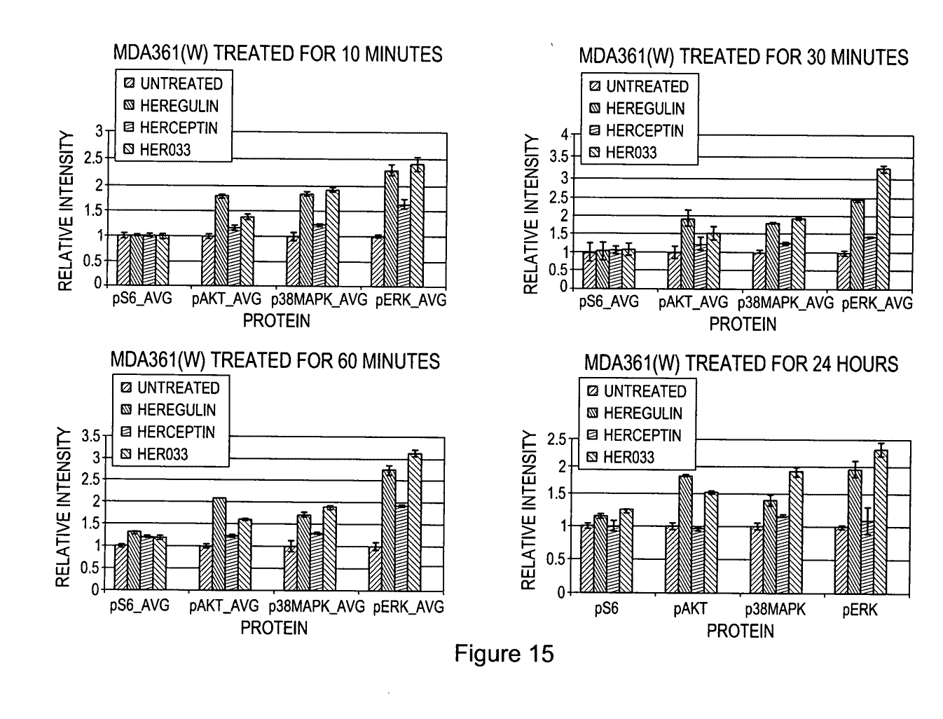

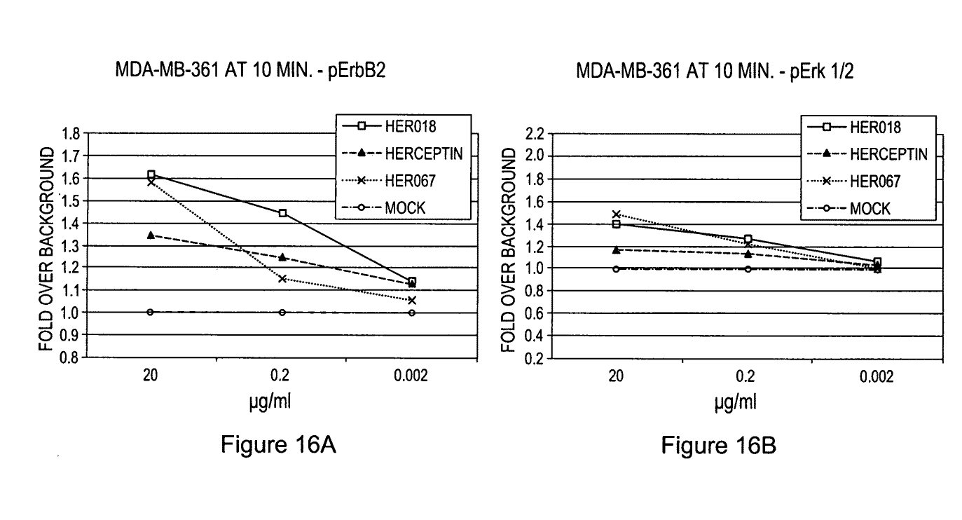

To measure increased downstream phosphoprotein signal transduction, MDA-MB-361 breast cancer cells were plated in 96-well format and treated with anti-Her2 or control reagents for the concentrations and times (10 min to 24 hr) shown in FIG. 15. Media was removed, cells washed with PBS, fixed with 4% paraformaldehyde, and permeabilized with 0.2% Triton 100/PBS. Cells were subsequently stained with either rabbit anti-pAKT (Cell Signaling Technologies), anti-pERK (Cellomics), anti-pS6K (Cell Signaling Technologies), or anti-p38MAPK (Cell Signaling Technologies). Following PBS wash (3×), cells were stained with secondary goat anti-rabbit IgG antibody labeled with ALEXA594. Cell fluorescence was quantified using Cellomics High Content assay at 594 nM.

Her067 (Her033) has agonistic activity (increased signaling) compared to trastuzumab (see Table 6). Moreover, Her067 and Her018 are generally a stronger inducer of Her2, Erk1/2, and Akt phosphorylation than trastuzumab. The increase was statistically significant as compared to the mock treatment when measured by the pairwise student T-test (<0.001).

TABLE 6
Induction of phosphorylation by HER018, HER067,
Herceptin and Heregulin

| MDA-MB-361 (JL) | HER018 | HER067 | Herceptin | Heregulin |
|---|---|---|---|---|
| phospho-ErbB2 | ++ | ++ | + | + |
| phospho-Erk1/2 | + | ++ | + | + |
| phospho-Akt | + | + | + | ++ |

We next investigated the effect of inhibiting kinase activity on SMIP anti-proliferative activity. Briefly, we seeded MDA-MB-361 breast cancer cells into 96-well plate format and grew them 48 hr in DMEM media plus 10% FBS. We then treated the cells either with 0.3 ug/ml of Her146 SMIP or vehicle (control). We additionally co-treated both Her146 and vehicle treated cells with the indicated dose of kinase inhibitor (for MEK:CL-1040 (PD184352); and for ERK1/2: FR180204). Cells were grown an additional 90 hr, media removed and fixed (4% paraformaldehyde) and stained with DAPI according to manufacture's protocol (Molecular Probes) and nuclei of viable cells counted using Cellomics High Content assay.

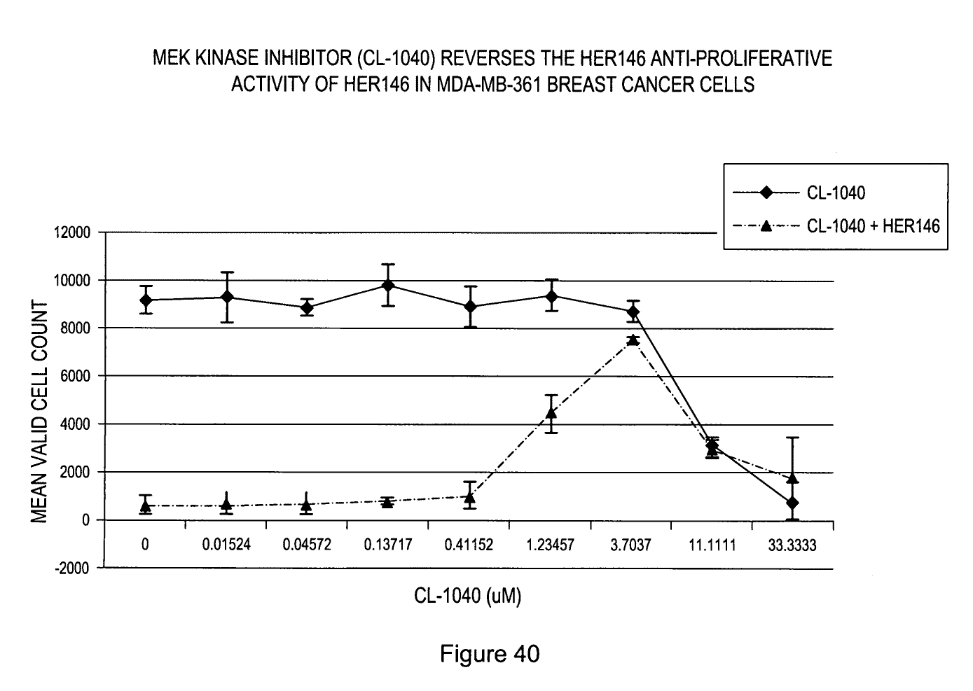

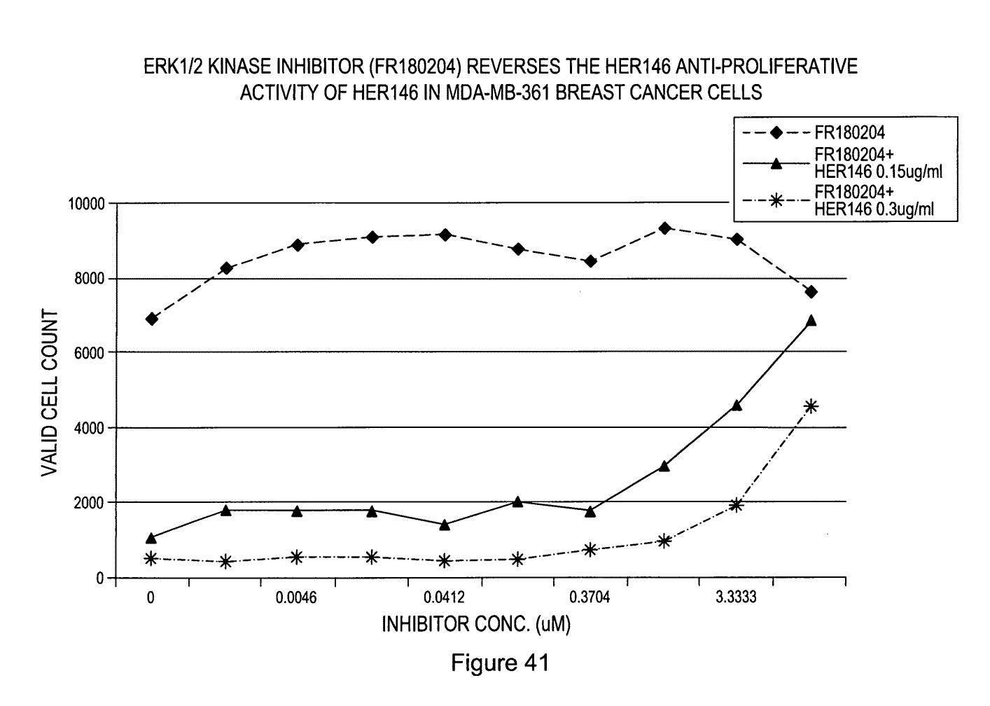

The results are shown in FIGS. 40 and 41. Her146 mediated anti-proliferative activity is demonstrated by decrease in viable cell count in absence of co-treatment with the kinase inhibitor. Inhibition of MEK with small molecule kinase inhibitor CL-1040 between 0.4 and 3.7 uM demonstrate dose dependent reversal of the Her146 mediated anti-proliferative activity, demonstrating that Her146 activity is mediated by hyperactivation of MEK kinase pathway activity. Higher doses of CL-1040 inhibited cell proliferation by complete inhibition of MEK kinase activity. Similarly, inhibition of ERK1/2 activity (downstream target of MEK) with small molecule kinase inhibitor FR180204 demonstrates dose dependent reversal of the Her146 mediated anti-proliferative activity, demonstrating that Her146 anti-proliferative activity is mediated by hyperactivation of MEK/ERK pathway.

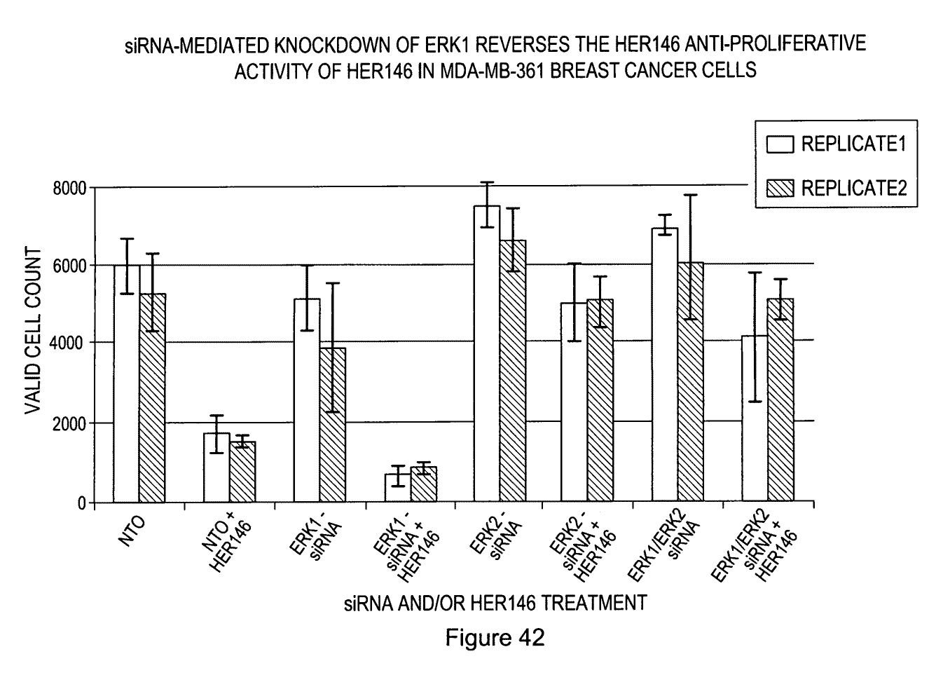

We also used siRNA against ERK1 or ERK2 to investigate the effect on SMIP anti-proliferative activity in MDA-MB-361 breast cancer cells. Briefly, the cells were reversed transfected with siRNA oligos (25 nM) targeting ERK1 or ERK2 kinases, or with non-targeting control oligo (NTO) using Dharmafect 4 lipid and following manufacture's recommended protocols in 96-well plate format. Cells were grown 60 hr in DMEM media plus 10% FBS and then treated with either Her146 (0.3 ug/ml) or vehicle control as indicated. Cells were then grown an additional 36 hr and media removed and cells fixed (4% paraformaldehyde) and stained with DAPI according to manufacture's protocol (Molecular Probes) and nuclei of viable cells counted using Cellomics High Content assay. Viable cell counts from two individual experiments are shown graphically in FIG. 42 as open or hatched bars. Treatment of MDA-MB-361 cells with Her146 in the presence of non-targeting siRNA (NTO) resulted in inhibition of cell proliferation. siRNA mediated knockdown of ERK1 kinase did not substantially alter the Her146 mediated anti-proliferative activity. In contrast, siRNA mediated knockdown of ERK2 kinase significantly reversed the Her146 antiproliferative activity. These data are consistent with reversal of Her146 anti-proliferative activity mediated by pharmacological inhibition of MEK or ERK1/2 and support the conclusion that these are the result of on-target activities. In addition, the results indicate that the hyperactivation of ERK2 but not ERK1 is responsible for cellular anti-proliferative activity of Her146.

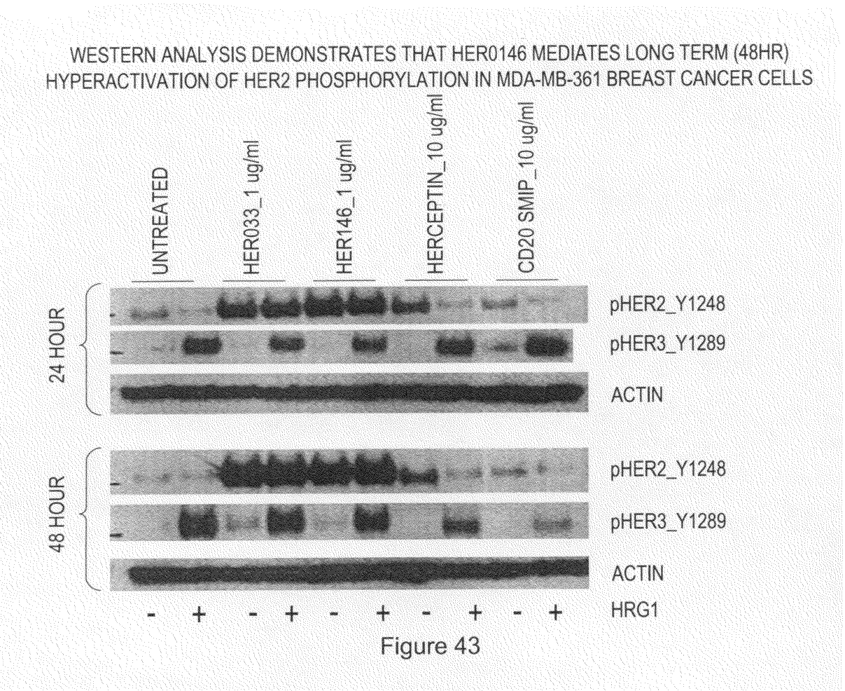

We further investigated the duration of HER2 phosphorylation by anti-HER2 SMIP. Briefly, MDA-MB-361 breast cancer cells were grown in DMEM media supplemented with 10% FBS. Cells were treated with either anti-Her2 SMIPs (Her033, Her146), Herceptin or controls anti-CD20 SMIP or untreated. Additionally, cell populations were either treated with heregulin (HRG1), the ligand activator of Her3, or vehicle for a total of 24 or 48 hr. Cells were harvested and protein lysates size fractionated by SDS-PAGE, and transferred to nitrocellulose membranes. Protein blots were probed with anti-pHer2 (Upstate), anti-pHer3 (Cell Signaling Technologies) or anti-Actin (Santa Cruz, loading control) monoclonal antibodies. Blots were subsequently stained with goat anti-rabbit IgG_coupled to horseradish peroxidase (Santa Cruz) and visualized by ECL staining (GE) following manufacture's protocol. As shown in FIG. 43, Her0146 mediates long term (48 hr) hyperactivation of Her2 phosphorylation in MDA-MB-361 breast cancer cells.

### Example 9

### Cell Cycle Assay

To investigate the effect of the ErbB2 ECD binder on cell cycle in HERCEPTIN® sensitive and HERCEPTIN® resistant cells, each well of a 6 well plate was seeded with 2×10⁵ cells (SKBR3 or BT474 (sensitive) or MDA-MB-453 or MDA-MB-361 (resistant)) and incubated overnight at 37° C./5% CO₂. Cells were then treated with antibody or SMIP (at 10 μg/ml final) (in triplicate) and incubated for another 24 or 48 hours. After incubation, the cells were pulsed with 50 uM BrdU (Sigma) for 30 minutes at 37° C., the media was removed, and the cells were treated with trypsin and harvested in a FACS tube on ice. The cells were washed with PBS, fixed with 70% cold ethanol, and incubated on ice for 30 minutes. The ethanol was removed and then 2N HCl/0.5% Triton X-100 was added, and the cells were incubated for 30 minutes at room temperature (RT). The acid was removed and neutralized with 0.1 M Na₂B₄O₇ for 15 min at RT. The neutralization buffer was removed, FITC labeled anti-BrdU antibody was added (BD Bioscience) in PBS/0.5% TWEEN® 20/1% BSA, and the cells were incubated for 30 minutes at RT in the dark. The FITC dye was removed, the cells washed, and then DAPI nuclear stain (Invitrogen) and RNAse A (Qiagen) each at 1:1000 dilution was added and the cells were incubated 15 minutes in the dark and then analyzed by FACS. Statistical analysis of the data was performed using ANOVA and Student's t-test.

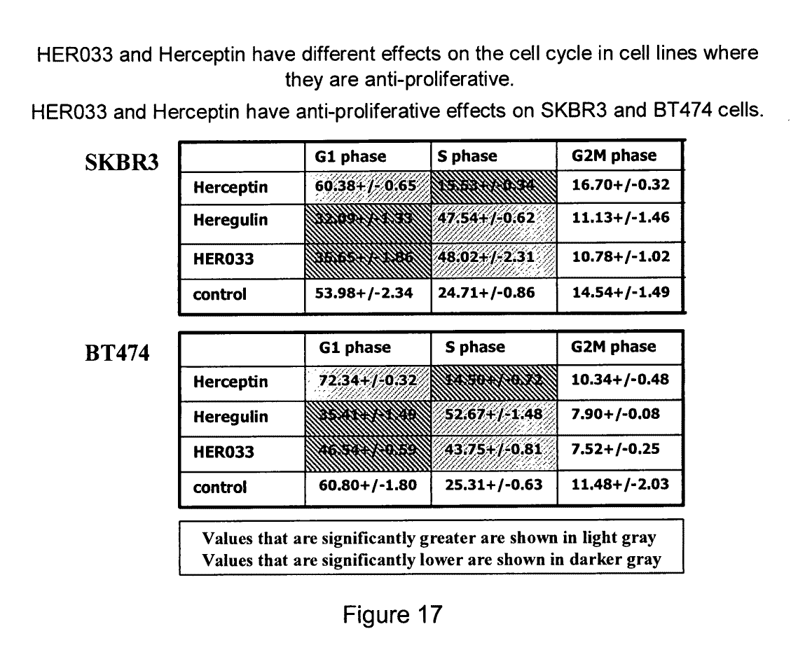

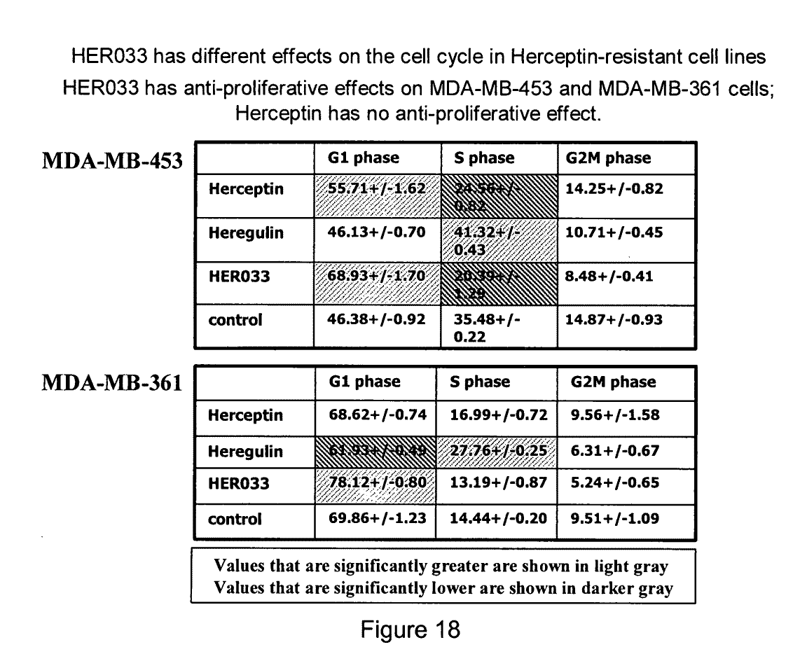

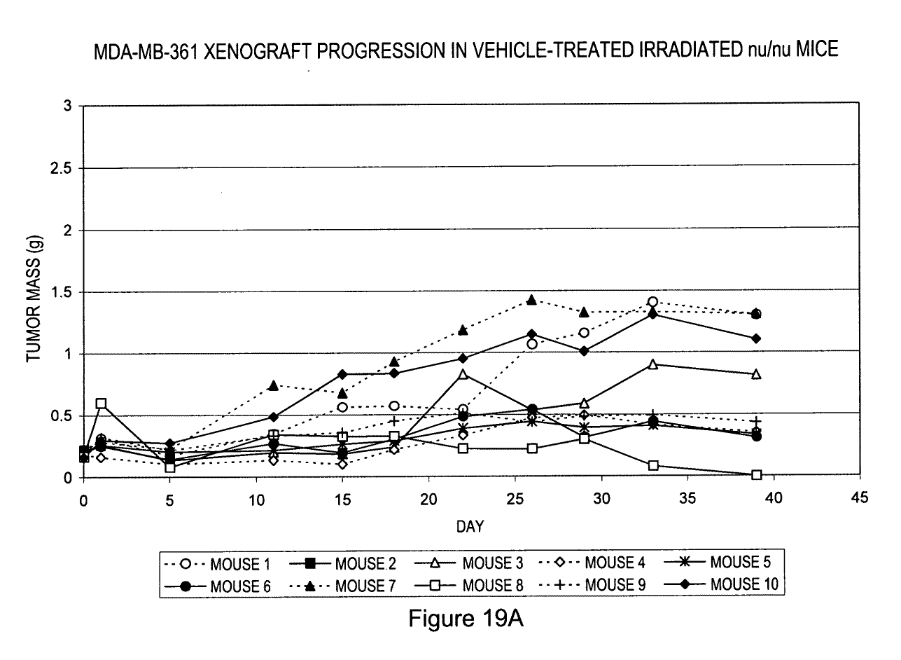

The results are presented in FIGS. 17 and 18. We observed an increased number of cells in the G1 phase in HERCEPTIN® treated SKBR3, BT474 and MDA-MB-453 cells. Among cells treated with HER033SMIP, we observed an increased number of cells in S phase in SKBR3 and BT474 cells.

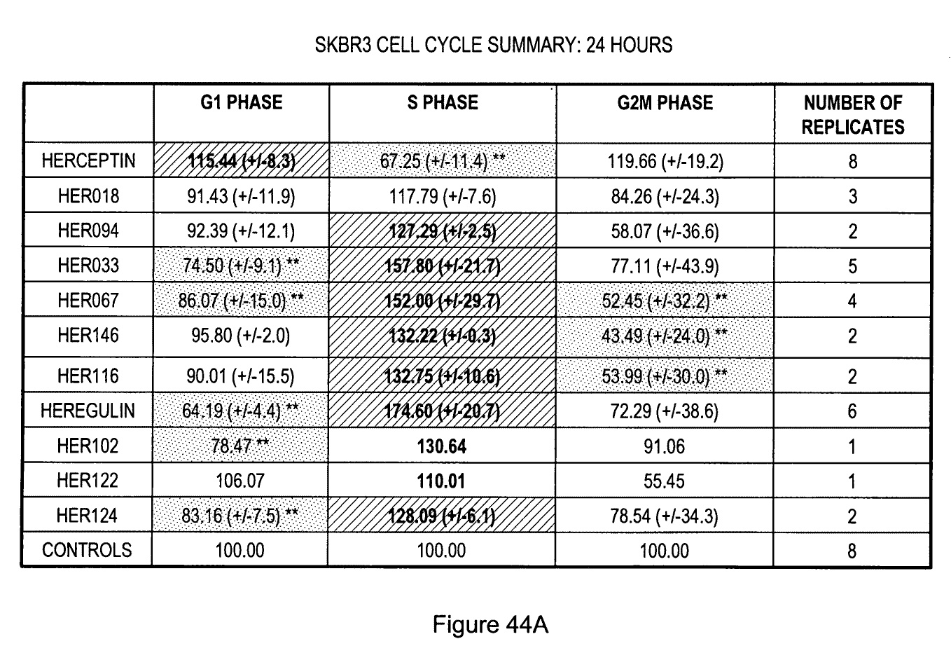

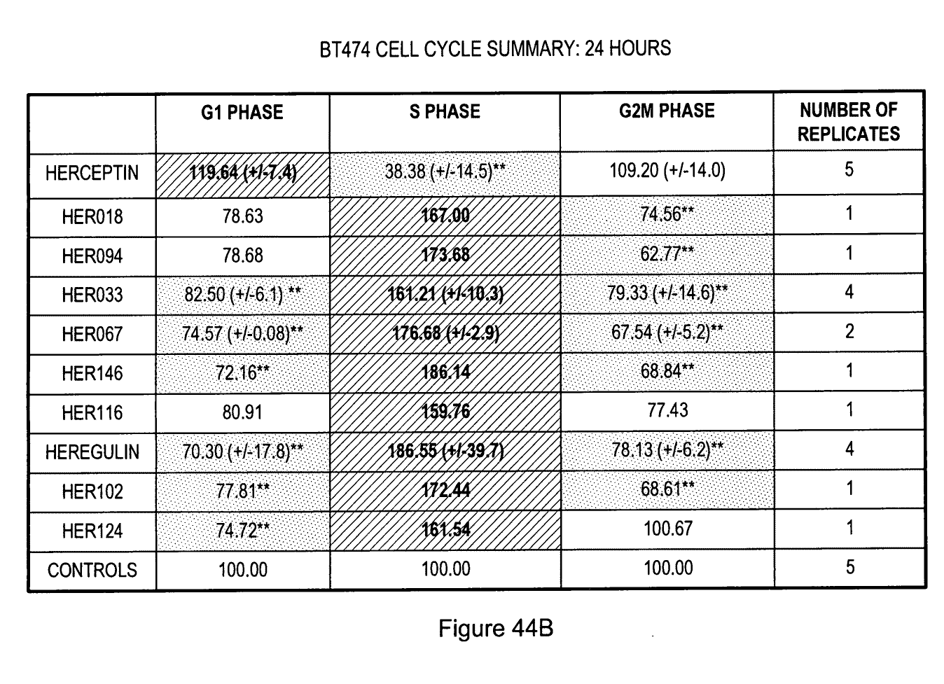

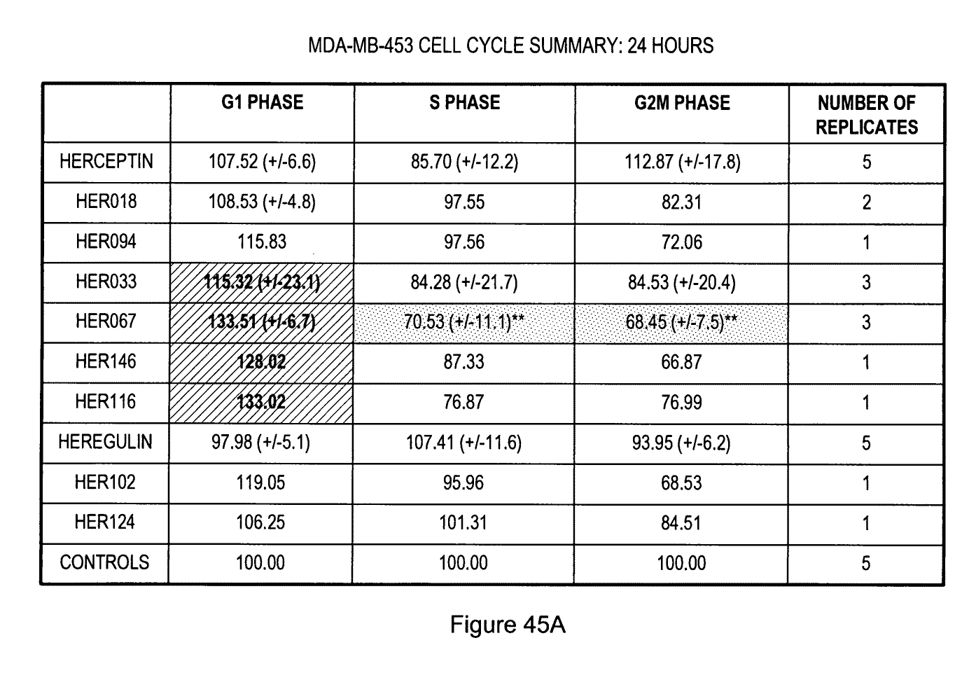

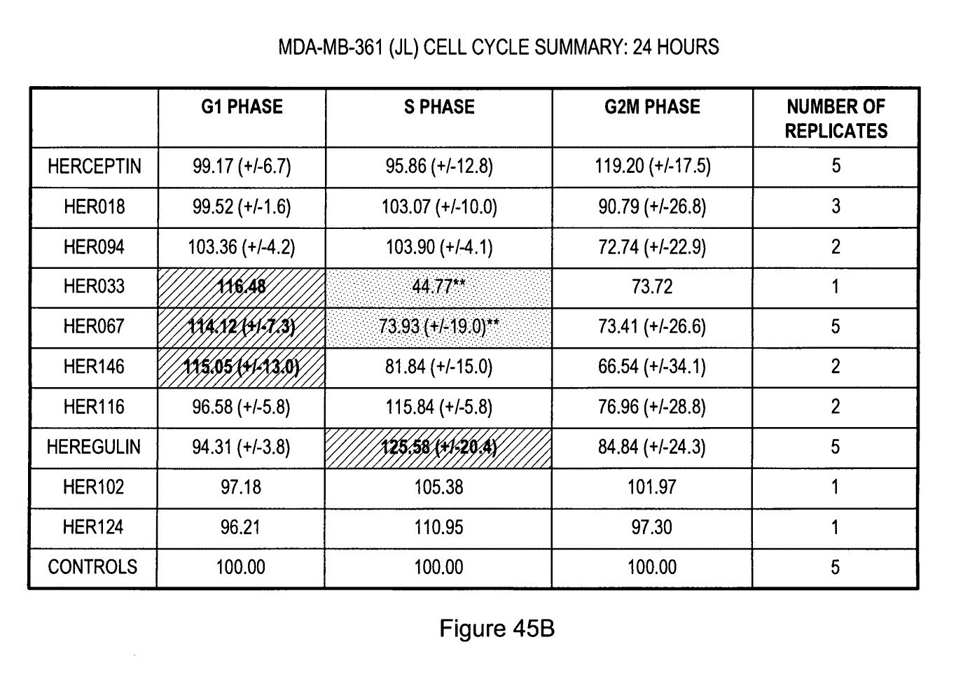

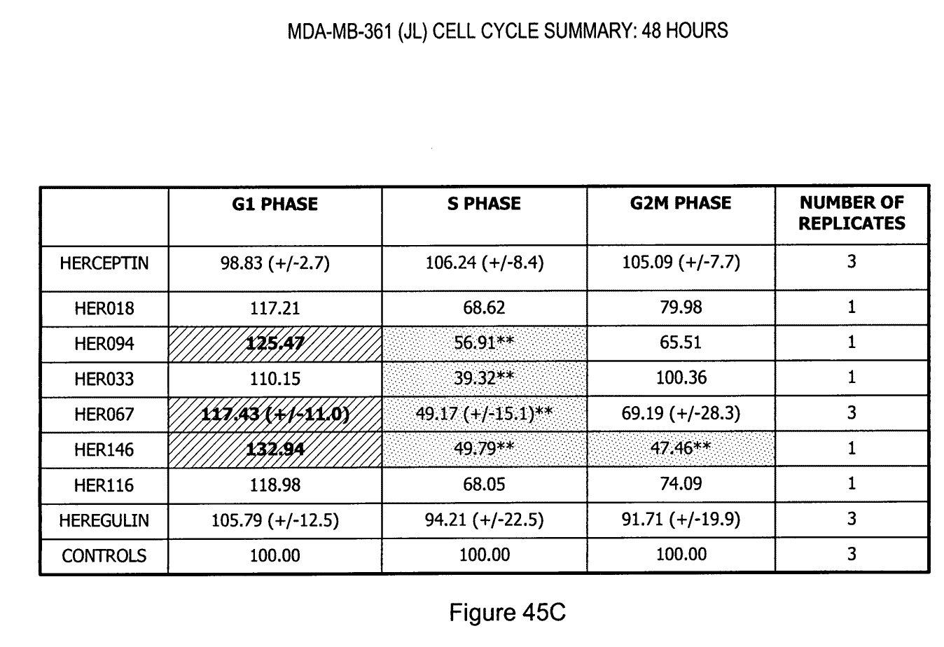

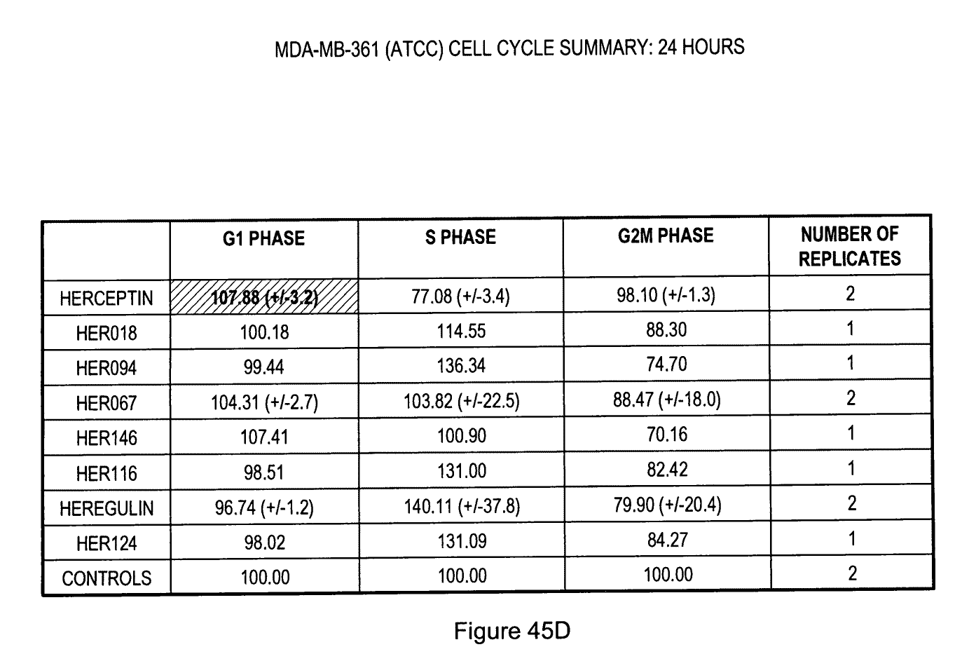

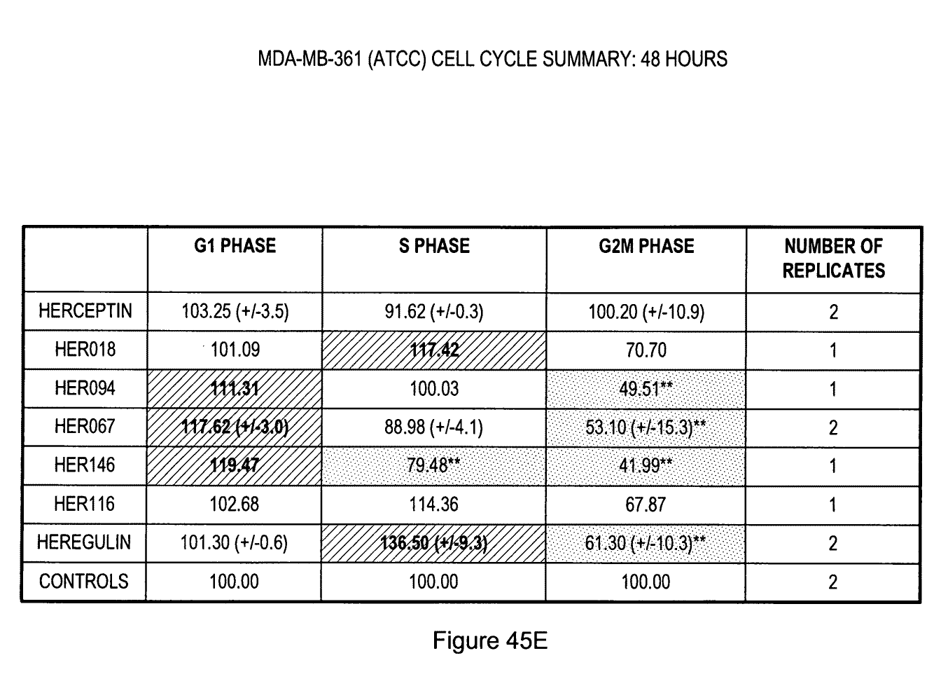

Additional results are presented in FIGS. 44A-B and 45A-E. Our results demonstrated that SMIPs have different effects on the cell cycle than Herceptin. While both Herceptin and SMIPs inhibited proliferation in SKBR3 and BT474 cells after 24 hours, Herceptin induced G1 arrest and SMIPs induced S-phase arrest. Additionally, while Herceptin did not inhibit cell cycle progression in MDA-MB-453, MDA-MB-361 (JL), and MDA-MB-361 (ATCC) cells after 24-48 hours, SMIP treatment inhibited the cell cycle by inducing G1 arrest. The cell cycle inhibition was not observed in MDA-MB-361 (ATCC) until 48 hours, but these cells grow slower than the other cell lines. Finally, HER116 appeared to behave a little differently than HER030/094, HER033/067, and HER146.

Specifically, we observed a decreased number of cells in the G1 phase in HER033, HER067, HER102, HER122 and Heregulin treated SKBR3 cells and in HER033, HER067, HER146, HER102, HER122 and Heregulin treated BT474 cells. We also observed an increased number of cells in the G1 phase in Herceptin® treated SKBR3 and BT474 cells; HER033, HER067, HER146, and HER116 treated MDA-MB-453 cells at 24 hours; HER033, HER067, and HER146 treated MDA-MB-361 (JL) cells at 24 hours; HER094, HER067, and HER146 treated MDA-MB-361 (JL) cells at 48 hours; Herceptin treated MDA-MB-361 (ATCC) cells at 24 hours; and HER094, HER067, and HER146 treated MDA-MB-361 (ATCC) cells at 48 hours.

Treatment with HER094, HER0333, HER067, HER146, HER116, HER124, and Heregulin resulted in an increase in the number of SKBR3 cells in S-phase at 24 hours. Treatment with HER018, HER094, HER033, HER067, HER146, HER116, HER102, HER124, and heregulin increased the number of BT474 cells in S-phase at 24 hours. We also observed an increase the number of cells in S-phase in Heregulin treated MDA-MB-361 (JL) cells at 24 hours; and HER018 and Heregulin treated MDA-MB-361 (ATCC) cells at 48 hours.

We observed a decreased number of cells in S-phase in Herceptin® treated SKBR3 and BT474 cells; HER067 treated MDA-MB-453 cells; HER033 and HER067 treated MDA-MB-361 (JL) cells at 24 hours; HER094, HER033, HER067 and HER146 treated MDA-MB-361 (JL) cells at 48 hours; and HER146 treated MDA-MB-361 (ATCC) cells at 48 hours.

HER067, HER146, and HER116 treatment decreased the number of SKBR3 cells in G2M phase. HER018, HER094, HER033, HER146, HER116, HER102, and heregulin decreased the number of BT474 in G2M phase. Alternatively, out of the Herceptin-resistant cell lines, only MDA-MB-361 (ATCC) cells at 48 hours showed significantly decreased G2M phase cells following SMIP treatment (HER094, HER067, HER146 and heregulin).

### Example 10

### In Vivo Xenograft Assay

To investigate the effect of the ErbB2 binding molecules of the invention in vivo, we tested the molecules in three mouse models.

SCID/Beige Mouse Model

Female (6-7 week old) Beige SCID mice (Beige SCID CB-17/IcrHsd-Prkdcscid-Lystbg) were obtained from Harlan Sprague Dawley, N.J. Virus free MDA-MB-361 cells were thawed from a new vial and cultured to generate appropriate numbers. Cells were grown to near confluency and had a viability of >90%. Cells were harvested, washed twice with sterile PBS, resuspended to 2×10⁸ cells/ml, then combined with Matrigel 1:2. and kept on ice until injection.

Tumor Cell Implantation and Monitoring: Each mouse was injected with 100 μl of the cell/Matrigel suspension (1×10⁷ cells) subcutaneously on the right flank. Mice were monitored daily for tumor growth. Tumors were established when they reached about 150 to about 300 mm³ (Volume=½[length×(width)²). Tumors developed in 100% of the implanted mice. Mice were sorted into groups according to tumor size, keeping means consistent among groups using LabCat software. Sorting occurred on day 0, which was the same day the mice received their first treatment.

Mice were monitored (i.e., weighed and tumors measured) two to three times weekly. Mice were sacrificed if ulceration of tumor occurred, extreme body weight loss (greater than or equal 20%), tumor exceeded about 1200 to about 1500 mm³, or tumor inhibited mobility of a mouse. The study is continued for a total of about 60 days.

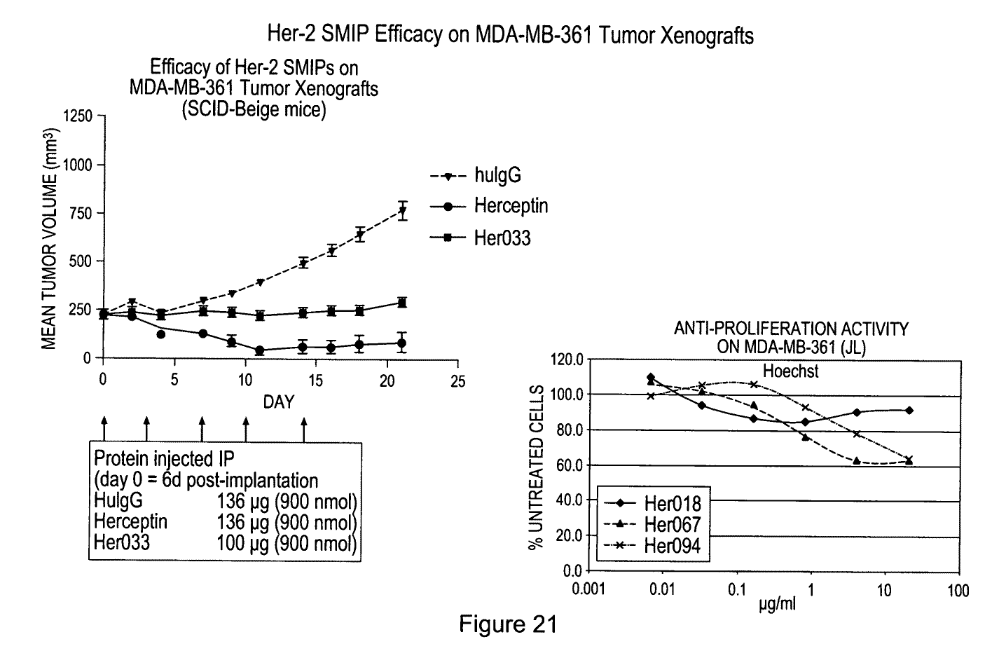

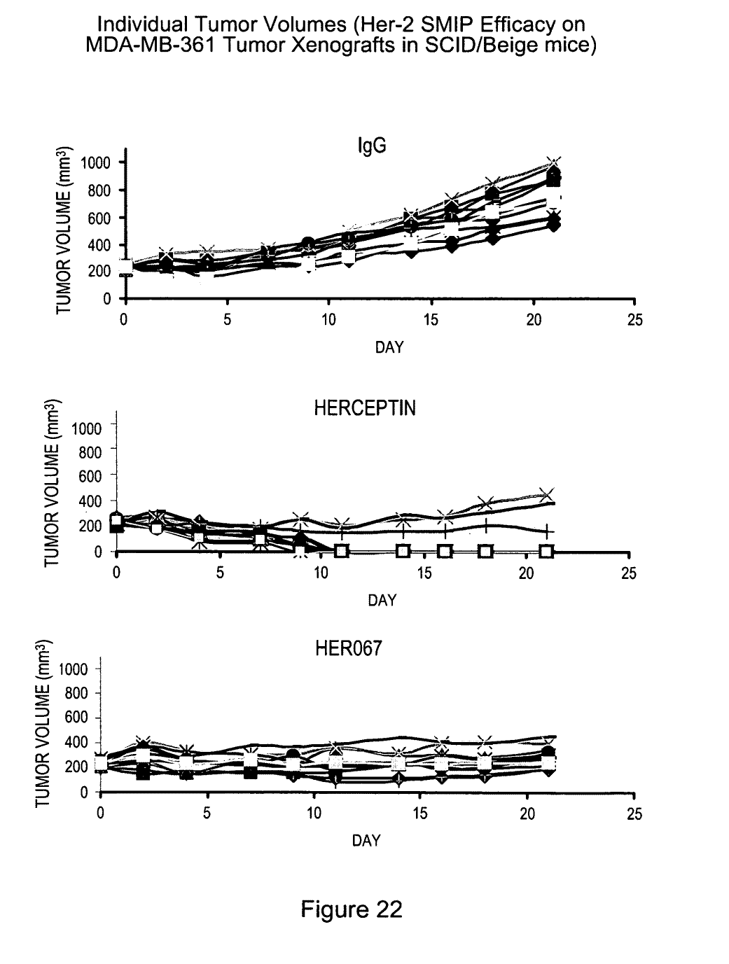

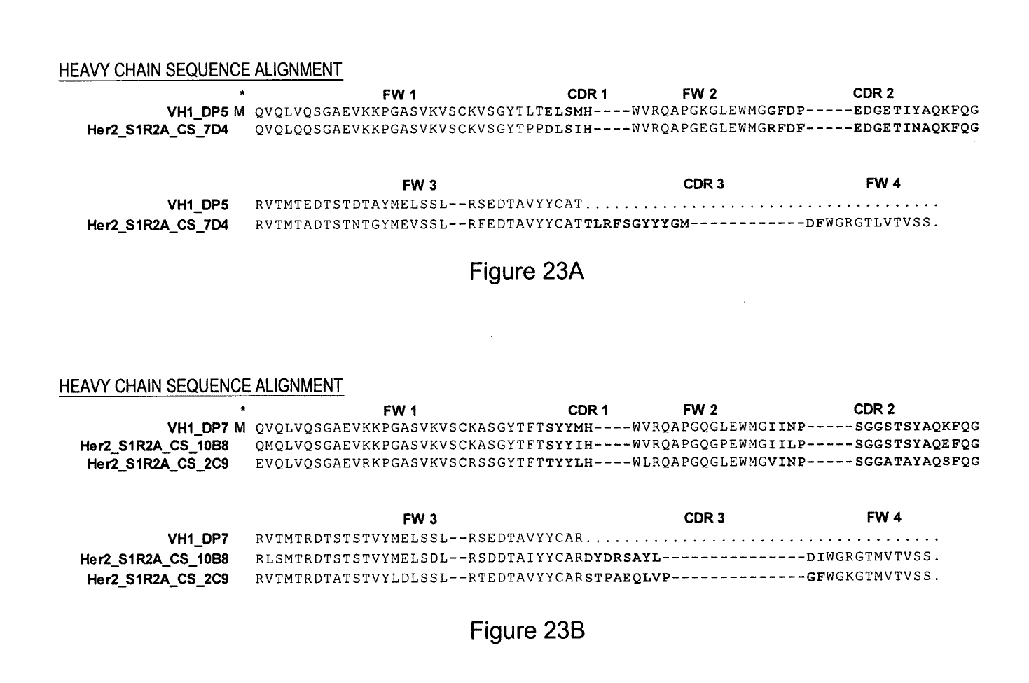

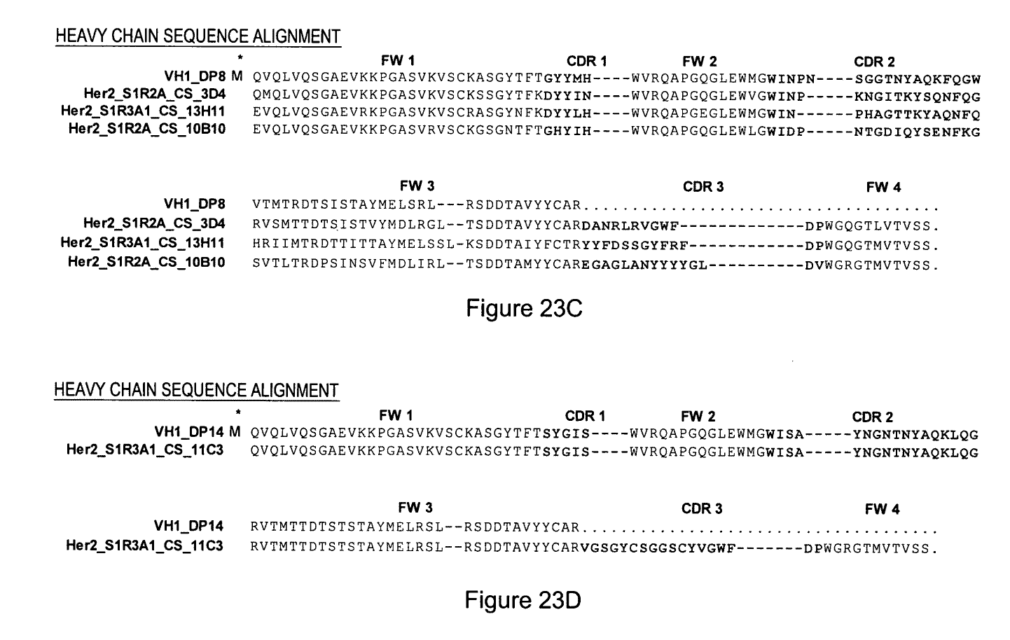

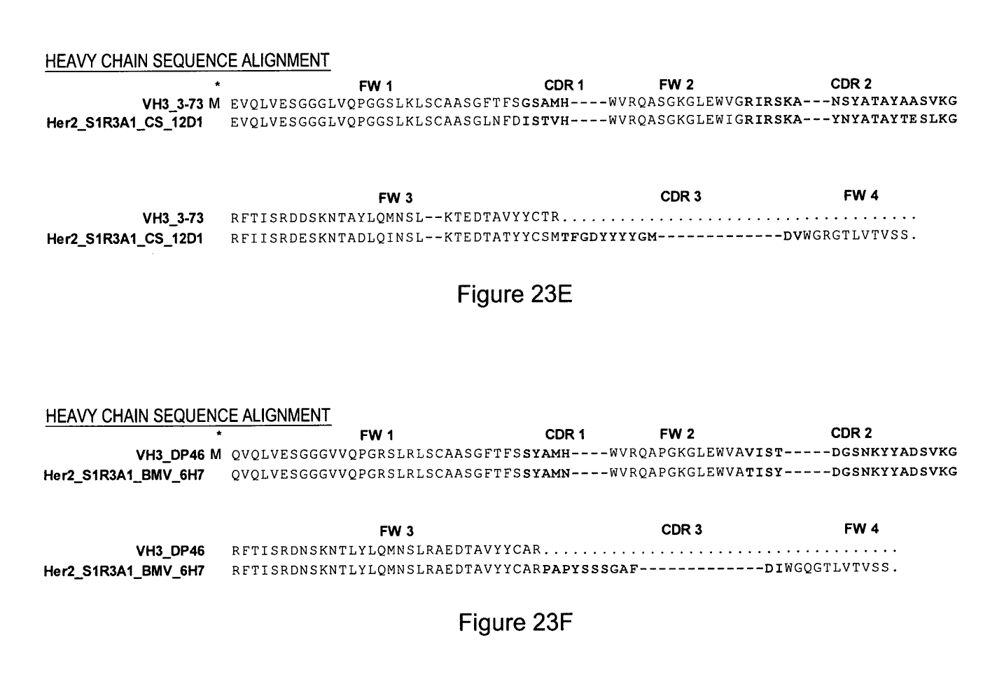

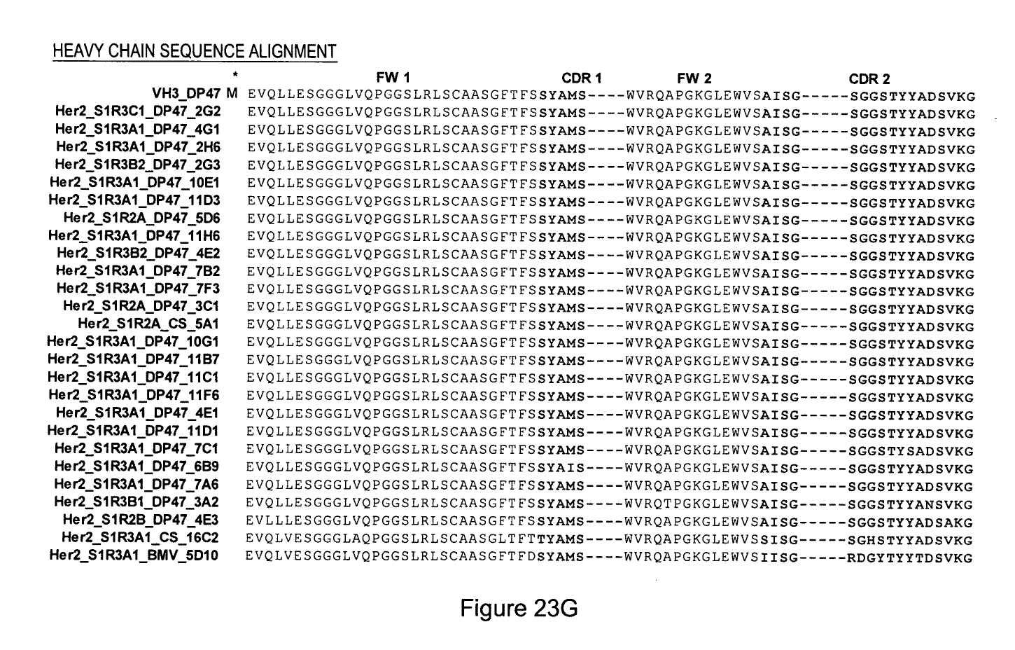

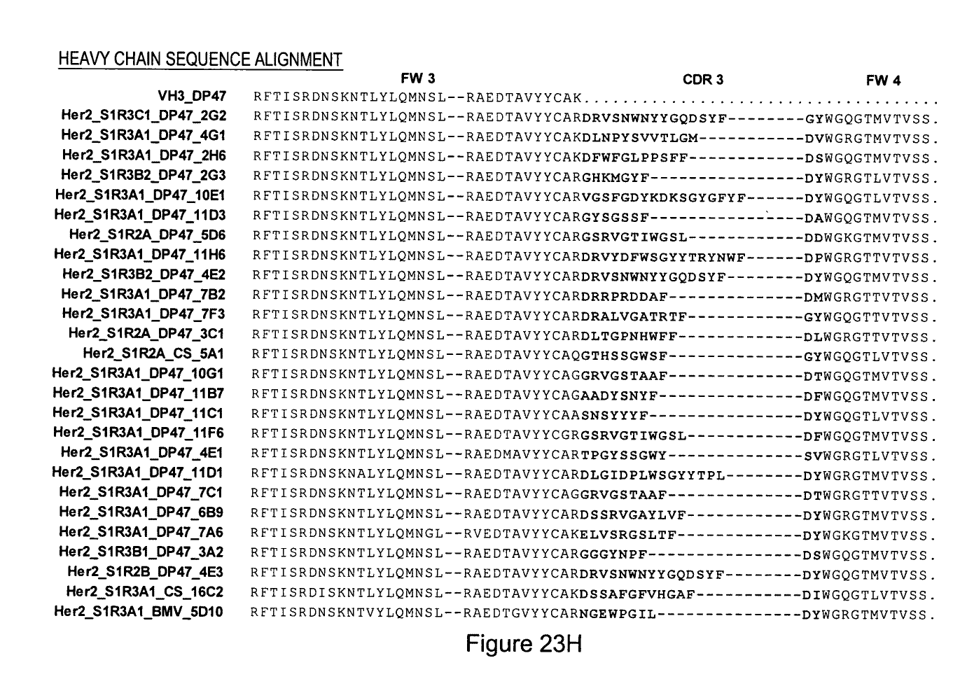

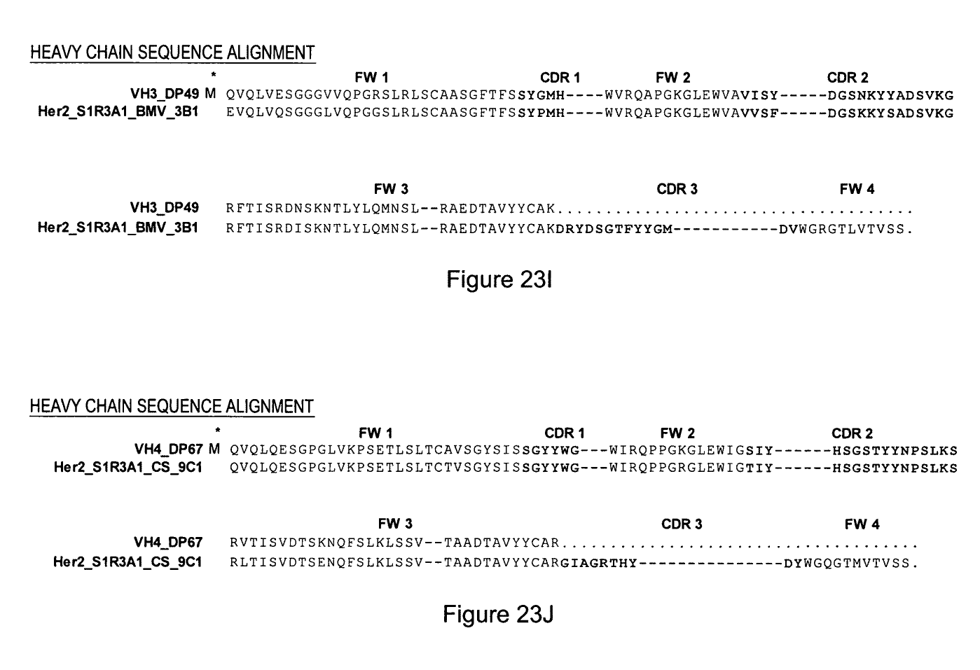

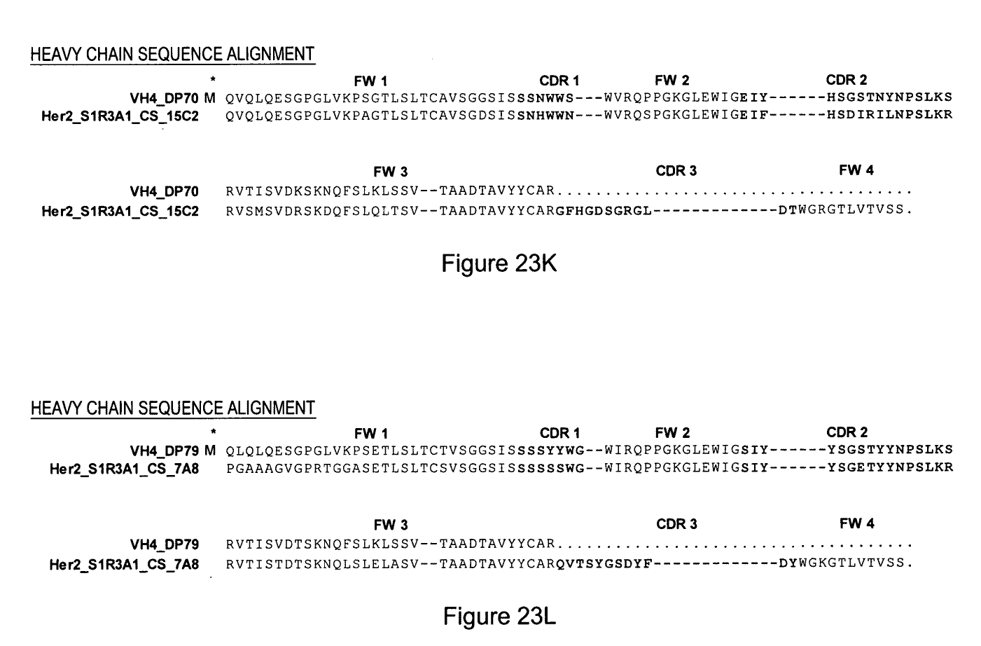

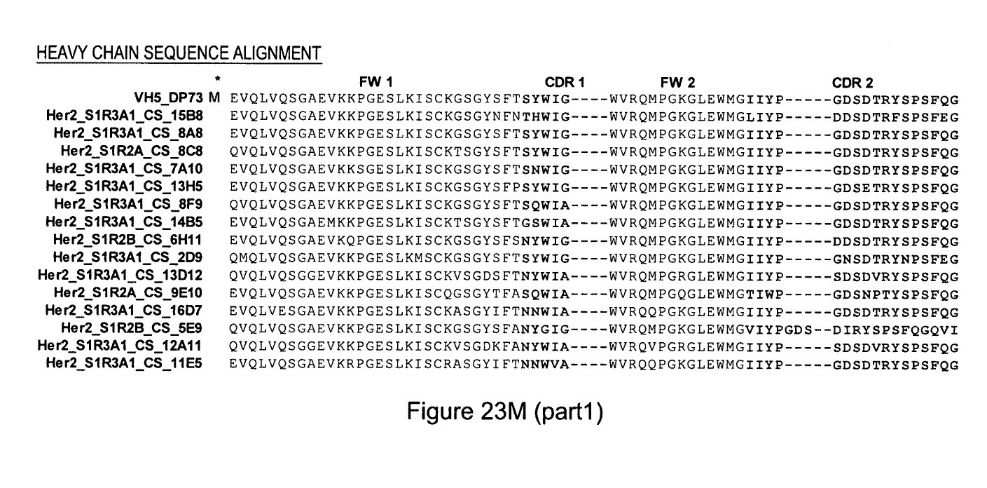

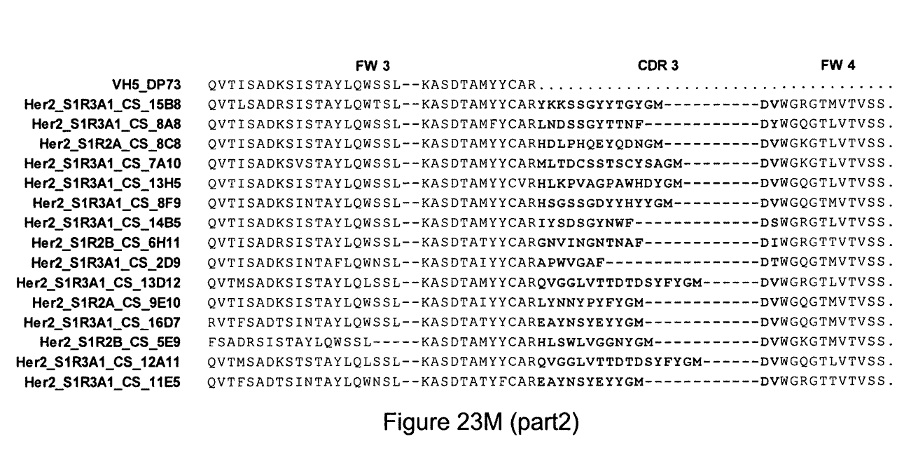

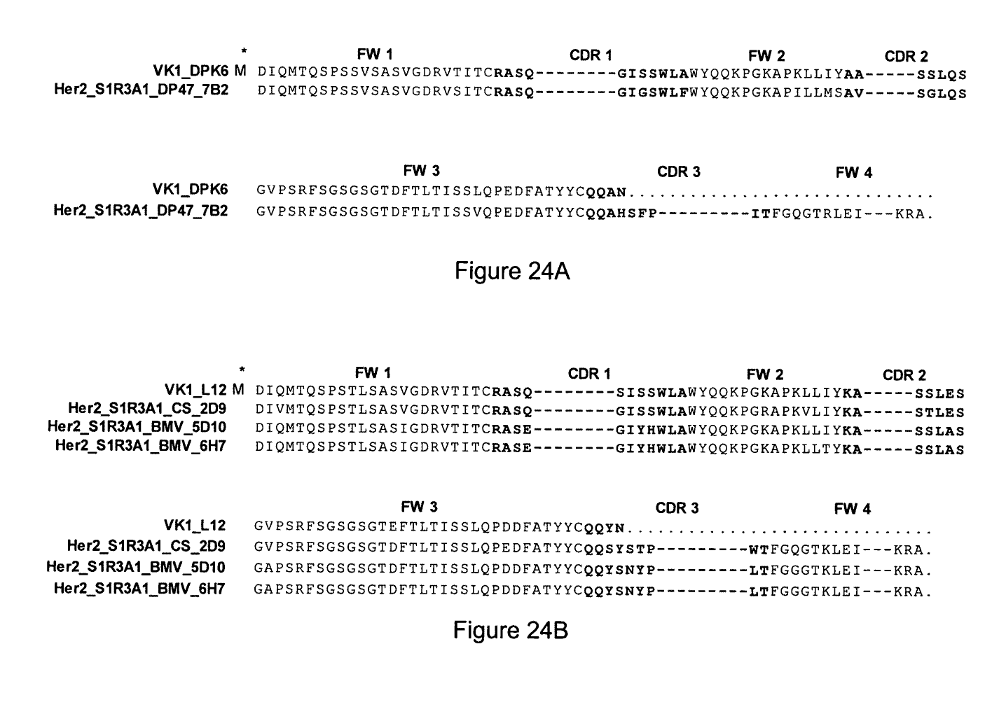

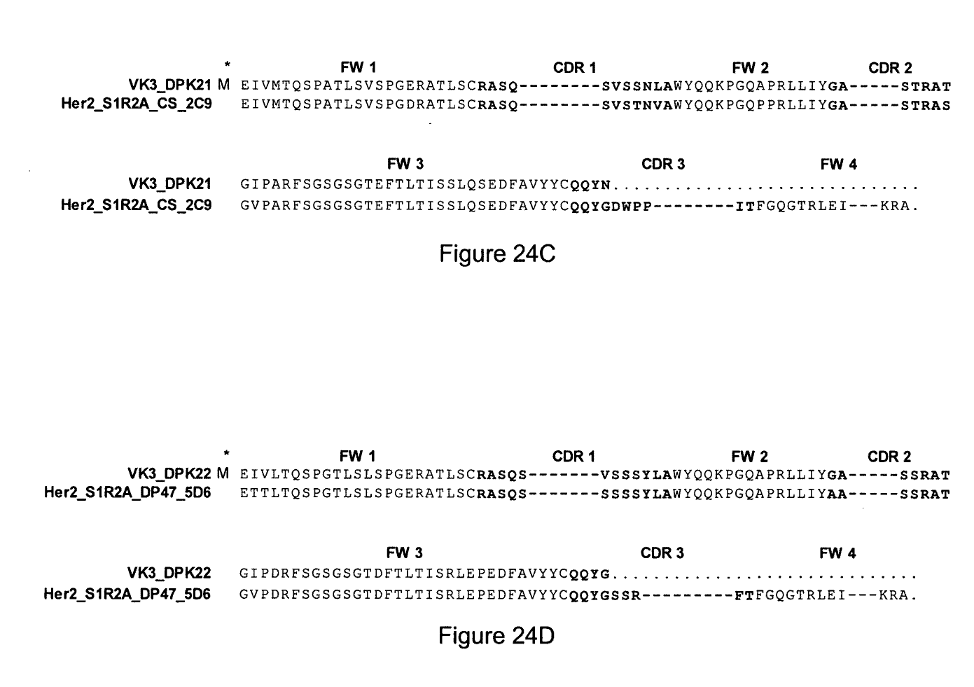

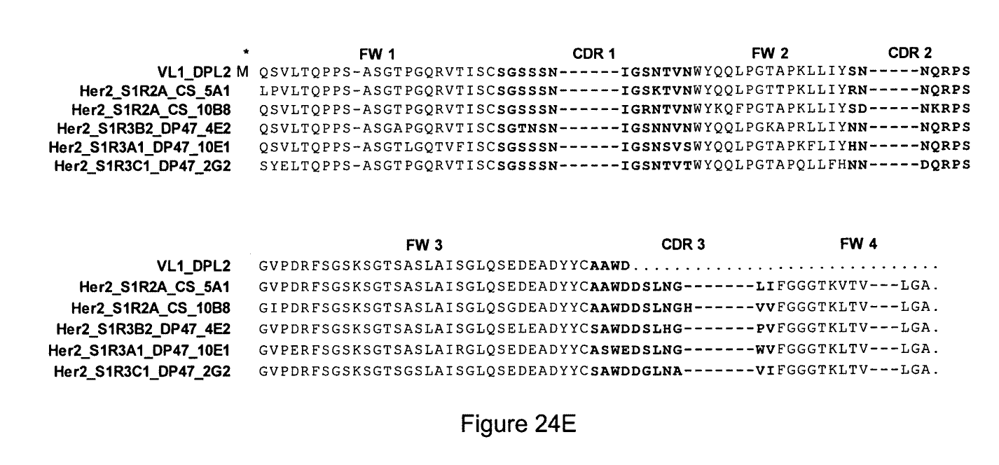

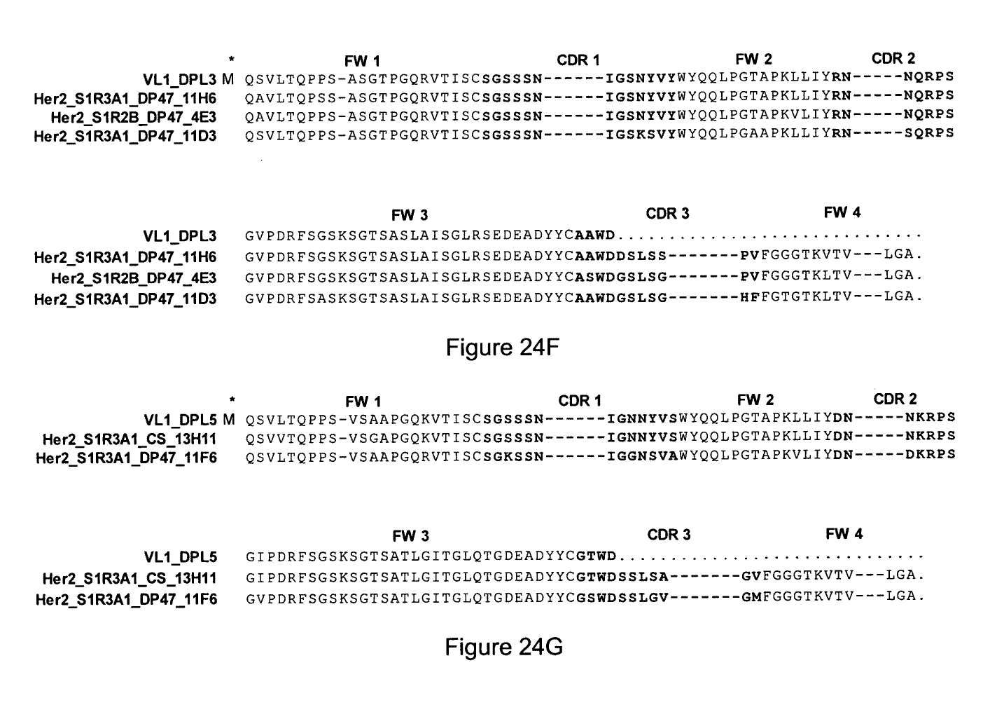

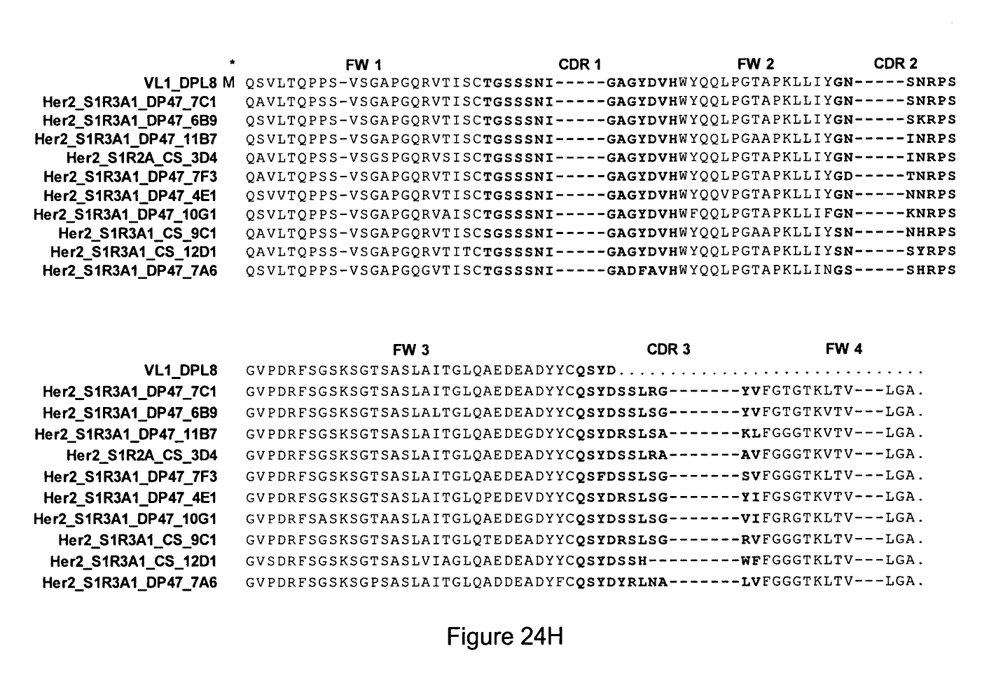

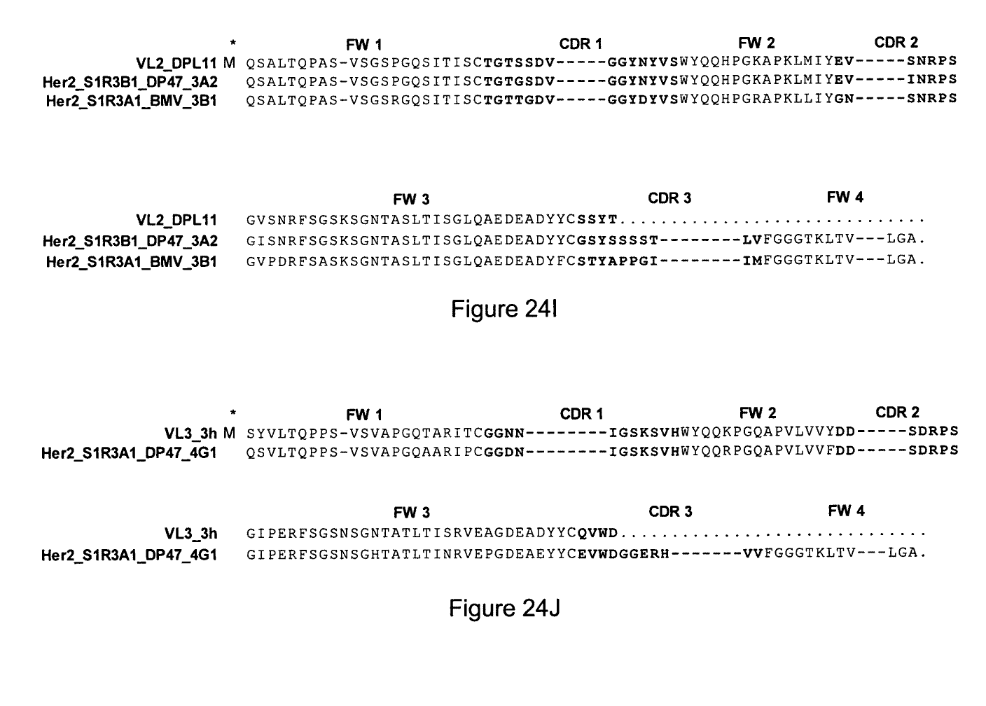

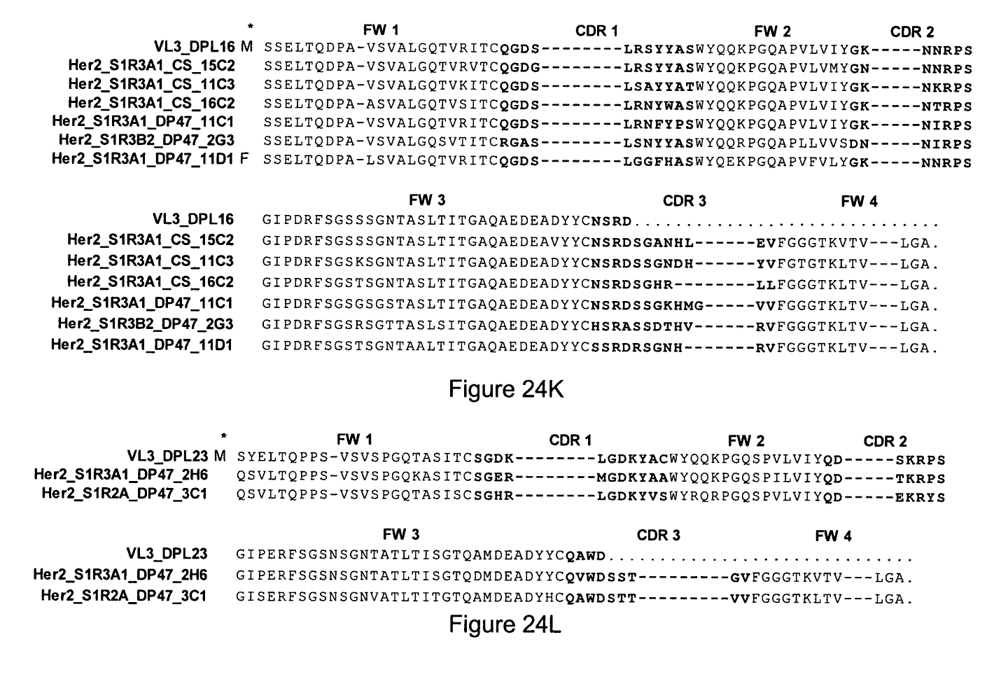

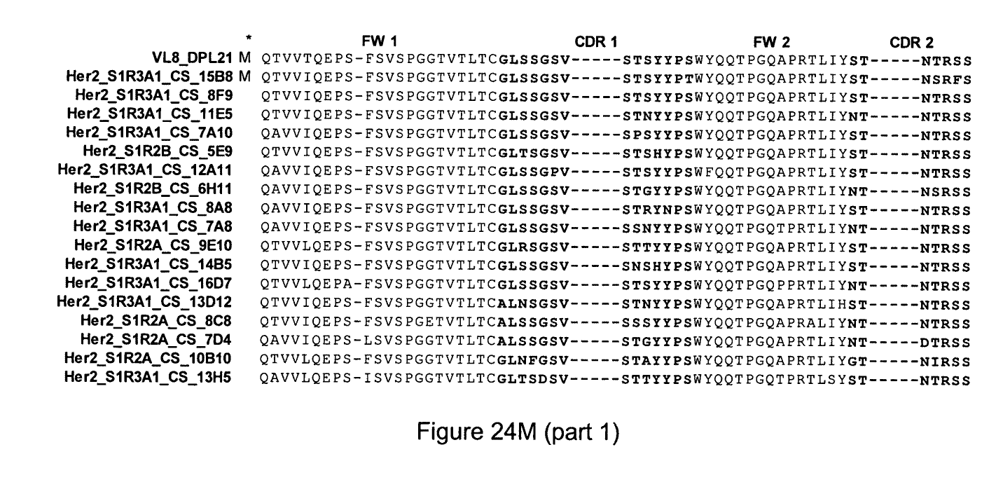

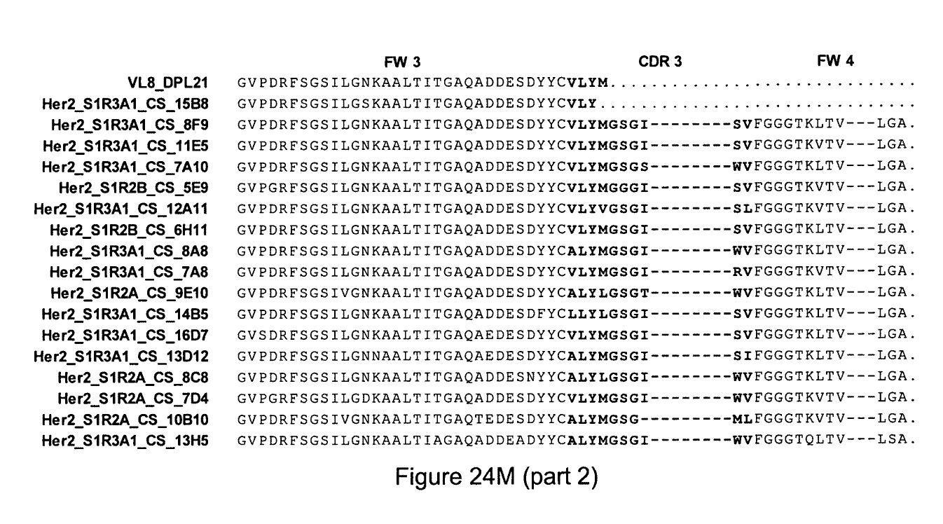

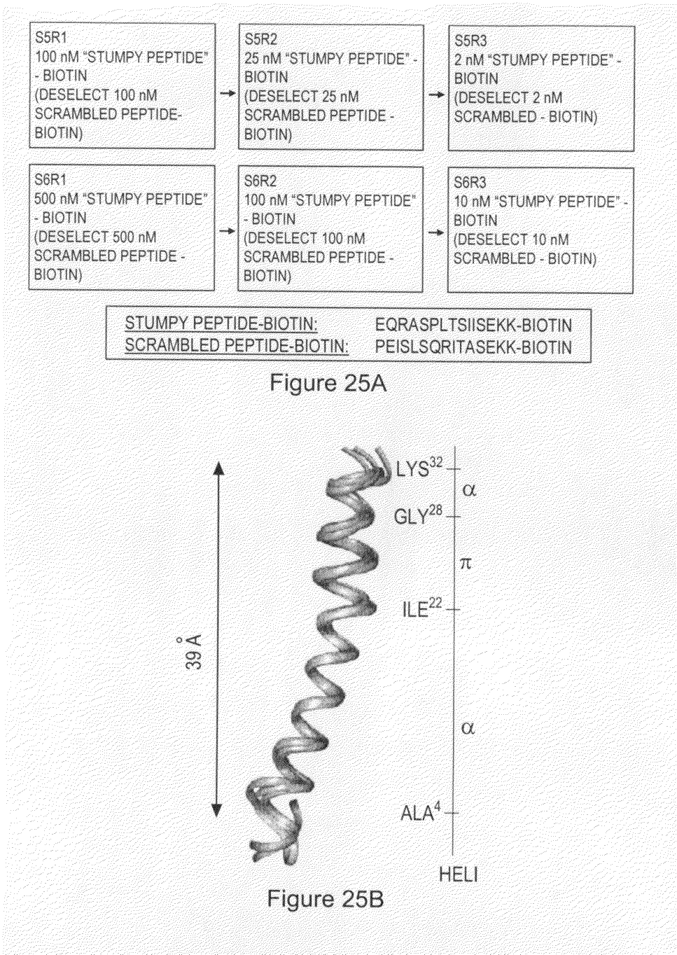

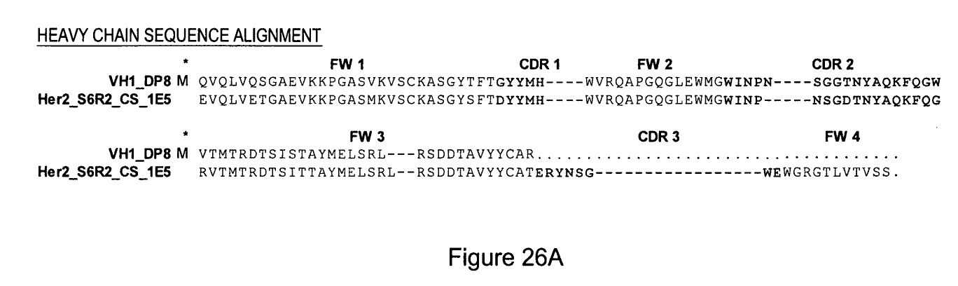

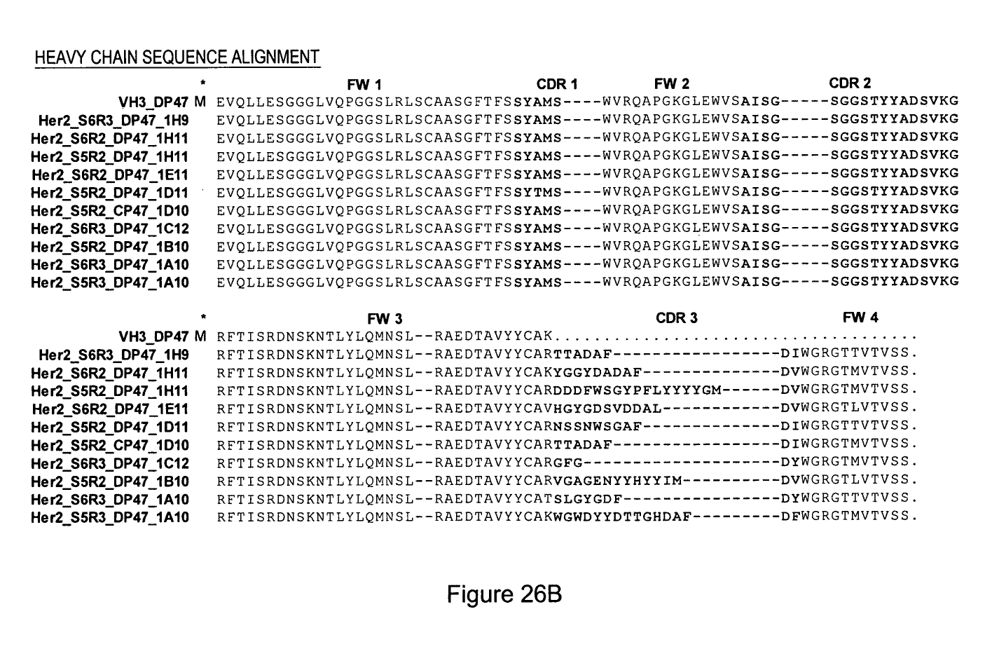

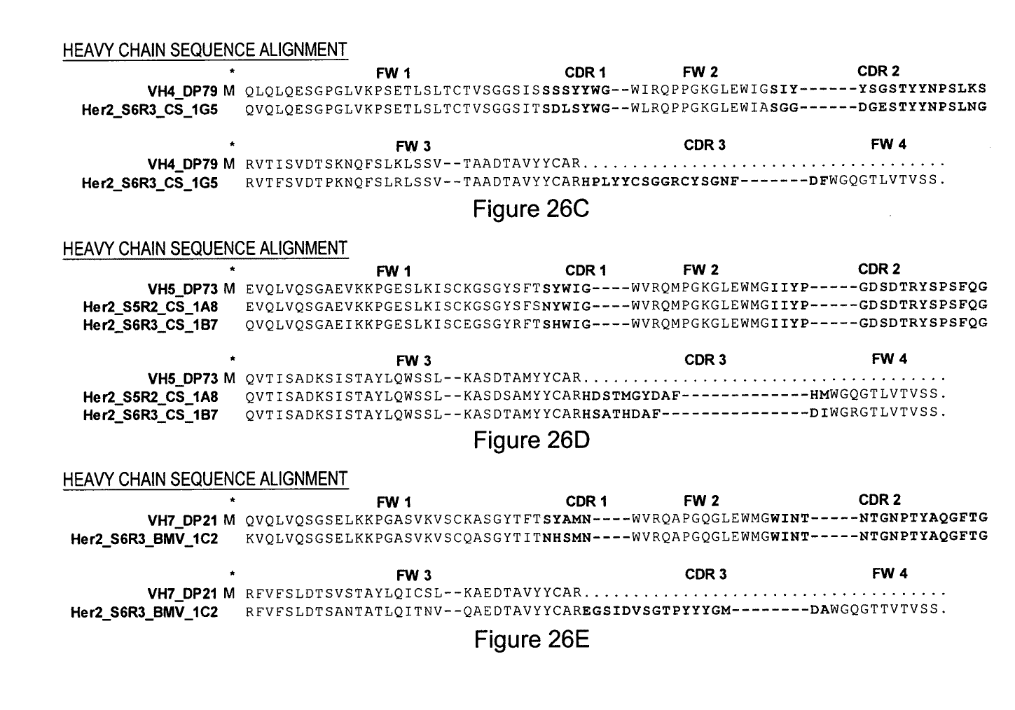

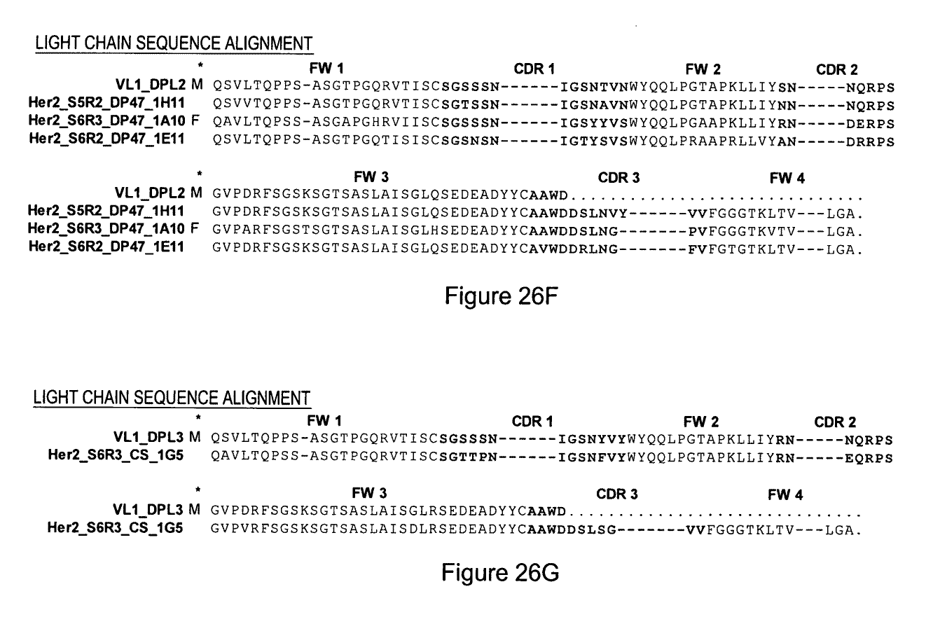

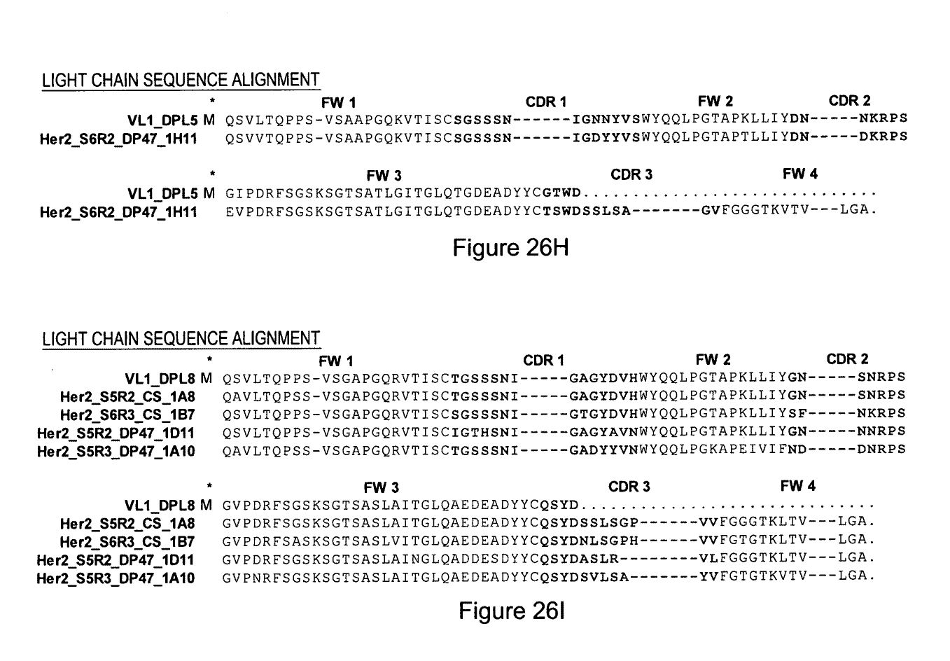

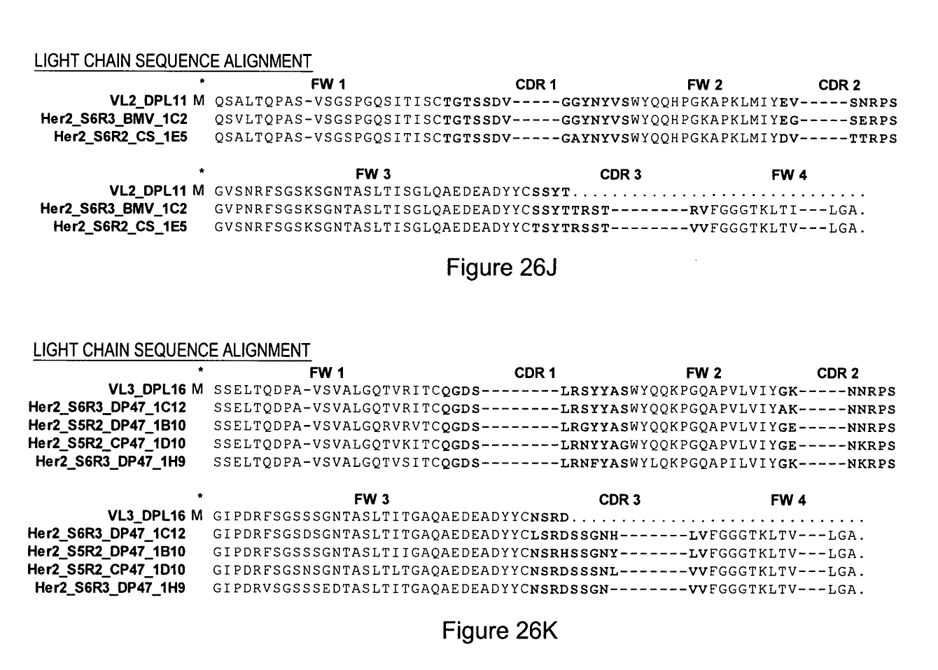

Treatment: Mice were sorted into three groups of 11 mice each. Treatment began on day 0 (about six days after cell implantation). Each mouse of a group received intraperitoneal treatments twice a week (for a total of five treatments), which were given in equimolar amounts (900 nM) of (1) SMIP HER067 (100 μg), (2) Herceptin (136 μg, positive control), or (3) human IgG (136 μg, negative control). Survival and tumor size was recorded two to three times weekly. Results were graphed (+/−SEM) and analyzed using Prism software (see FIGS. 21 and 22).

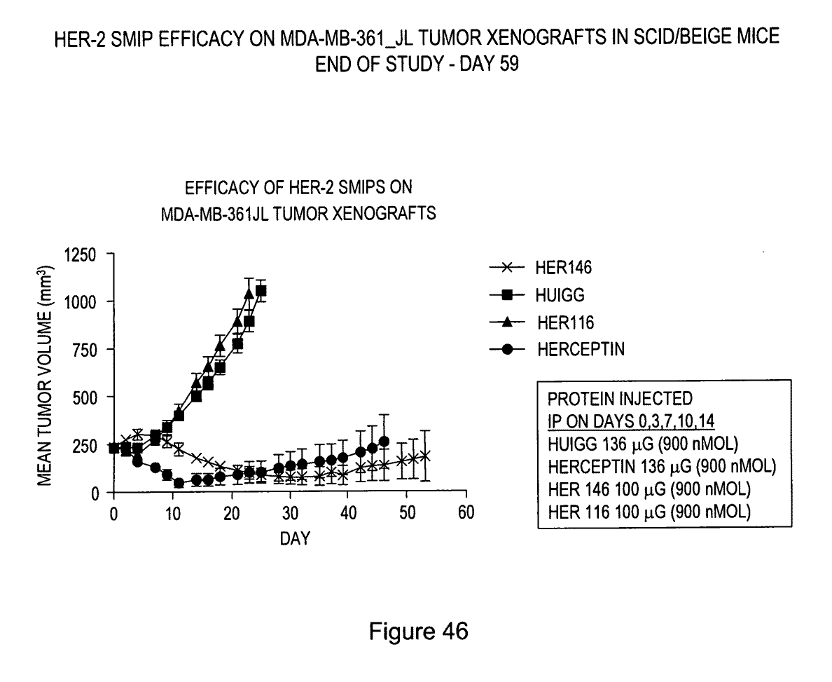

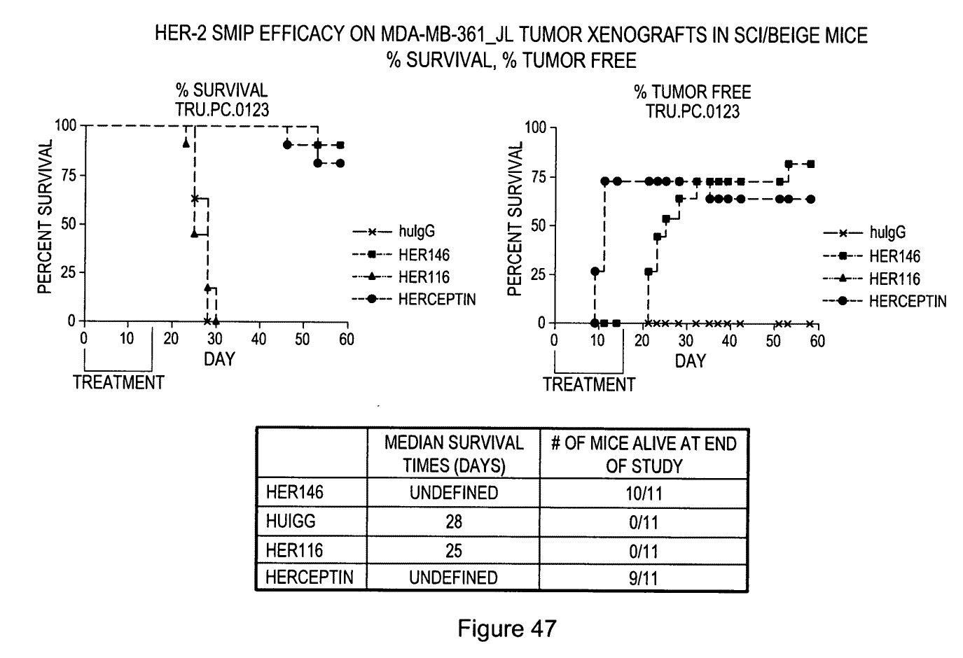

In a subsequent experiment, mice were sorted into 4 groups: (1) HER146 (100 μg), (2) HER116 (100 μg), (3) Herceptin (136 μg, positive control) and (4) human IgG (136 μg, negative control). Survival and tumor size was recorded two to three times a week. Results were graphed (+/−SEM) and analyzed using Prism software (see FIGS. 46 and 47)

BALB/c nu and nu/nu Mouse Models

Male BALB/c nu/nu (nude) mice (18-23 g) and female nu/nu (nude) mice (18-23 g) were obtained from Charles River Laboratories, Wilmington, Mass.

Subcutaneous BCL Xenografts:

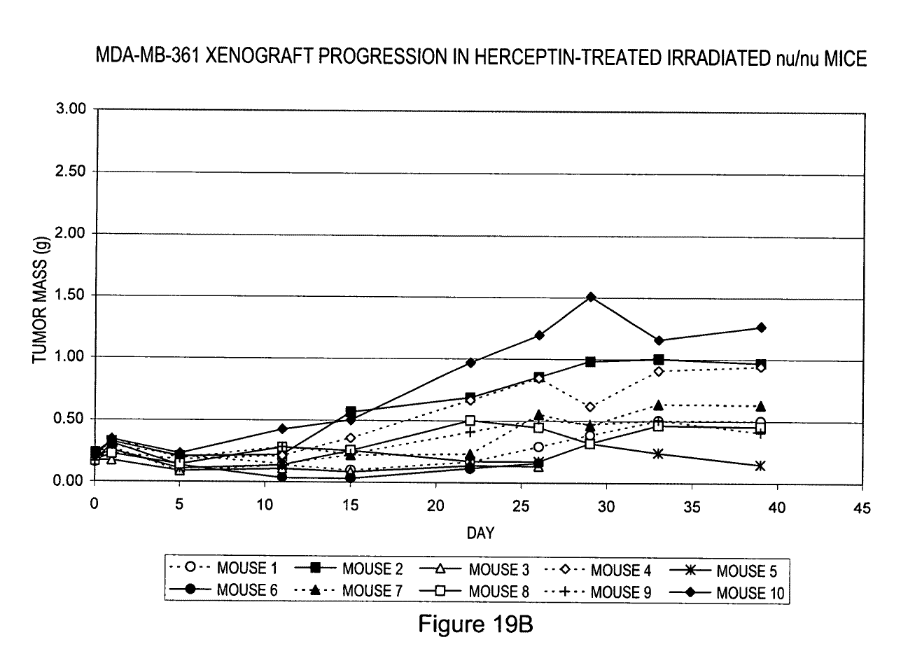

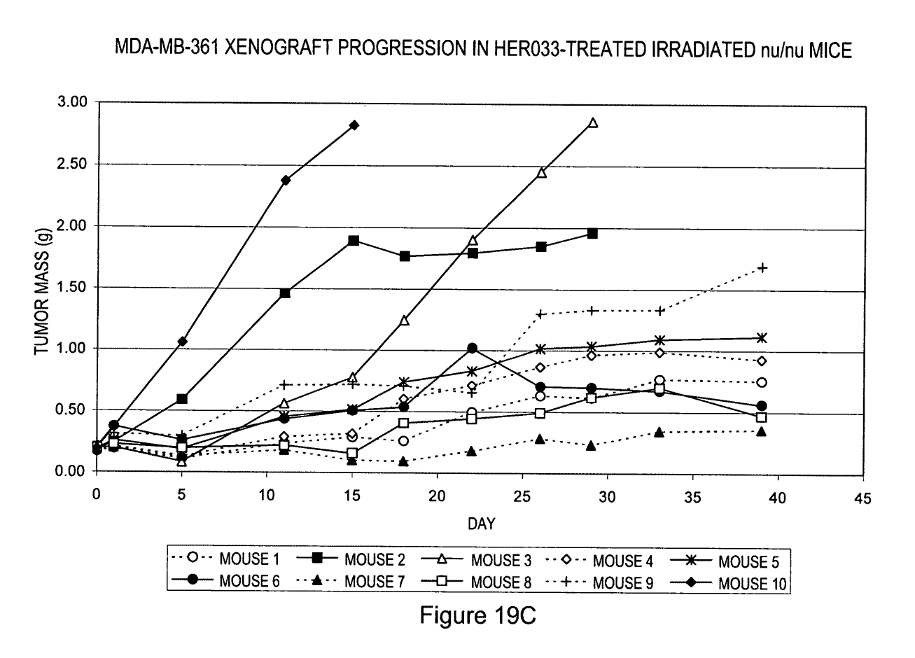

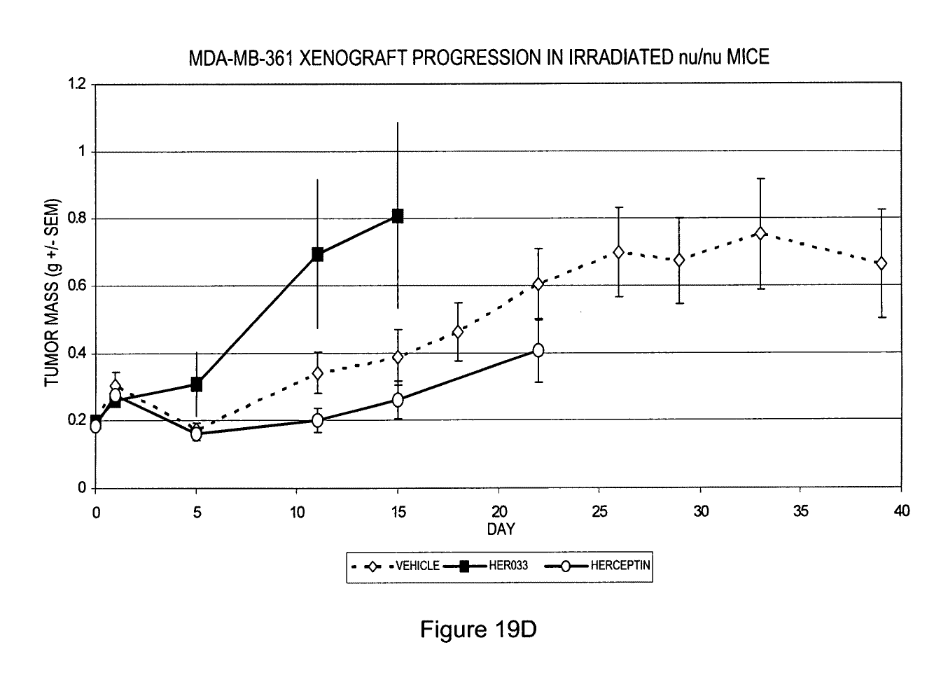

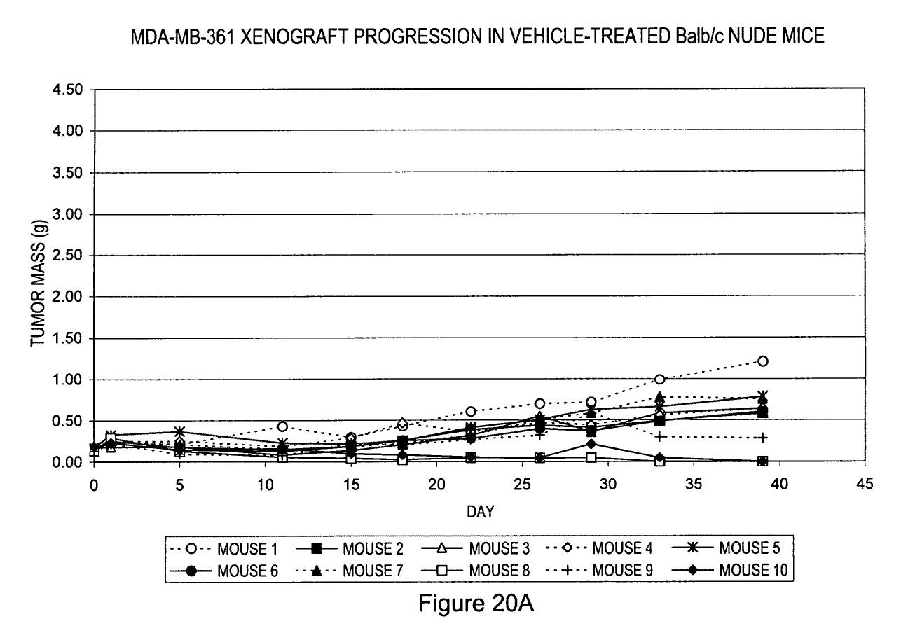

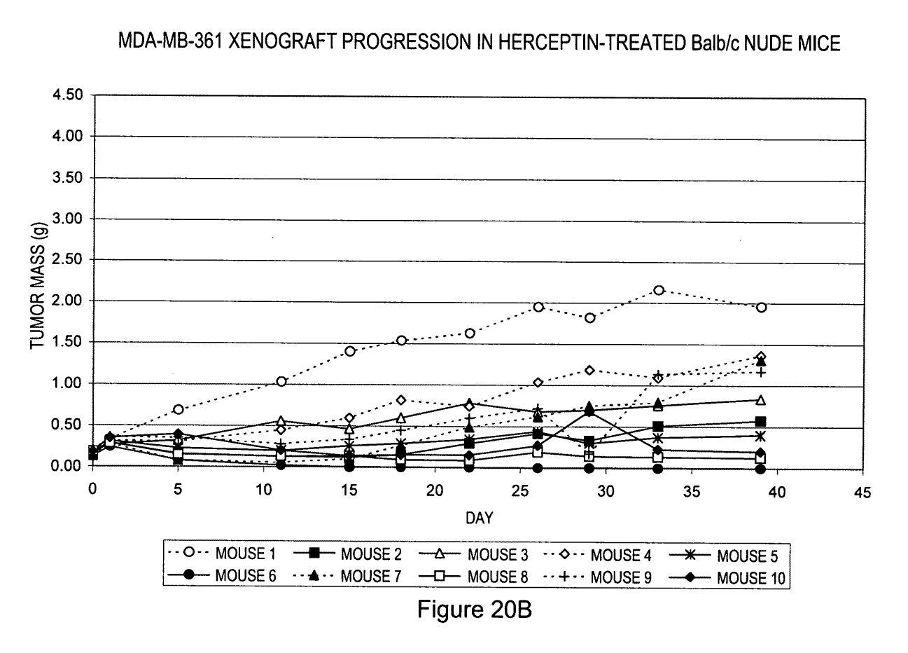

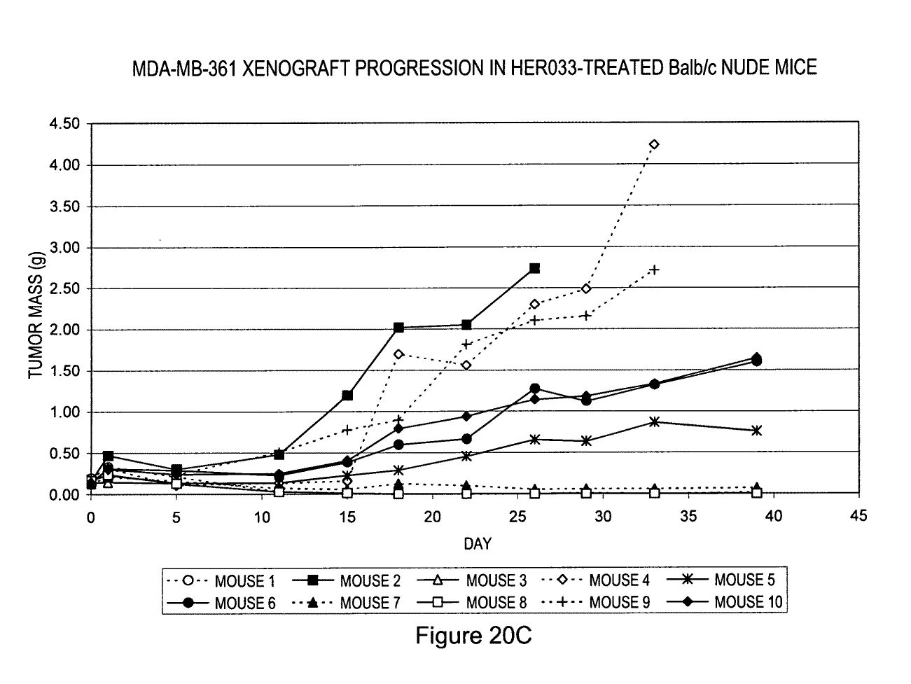

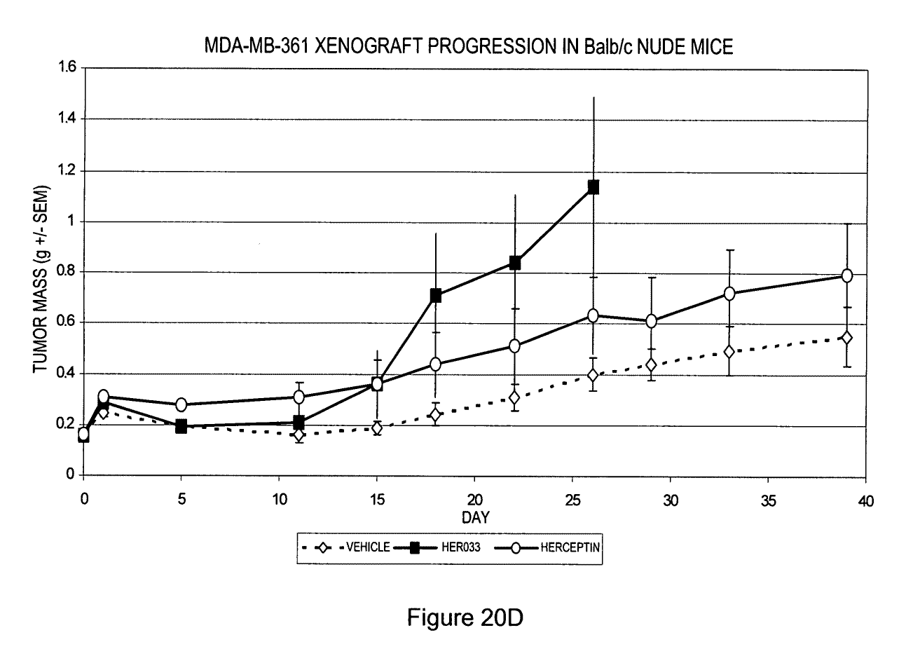

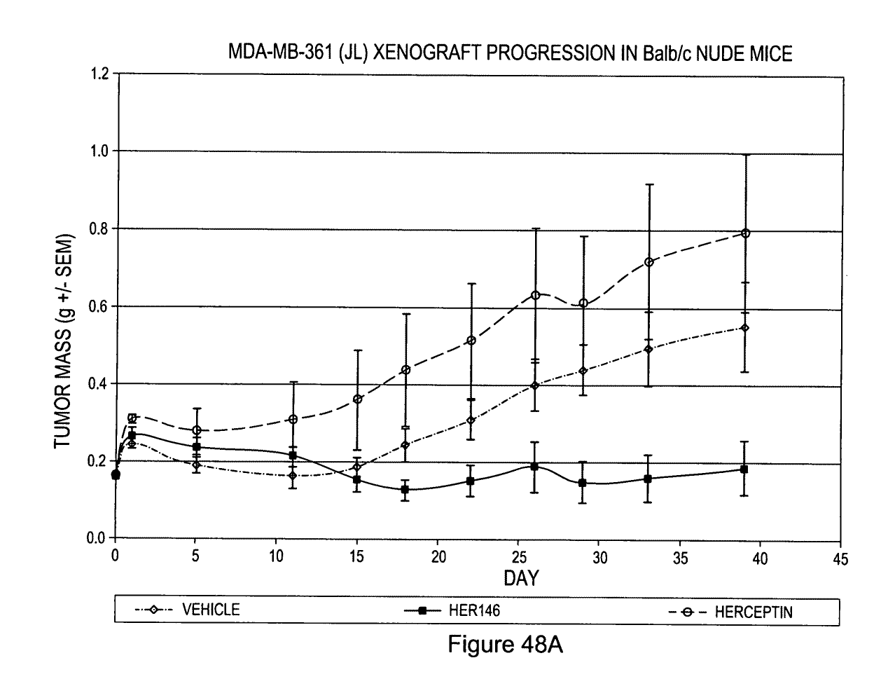

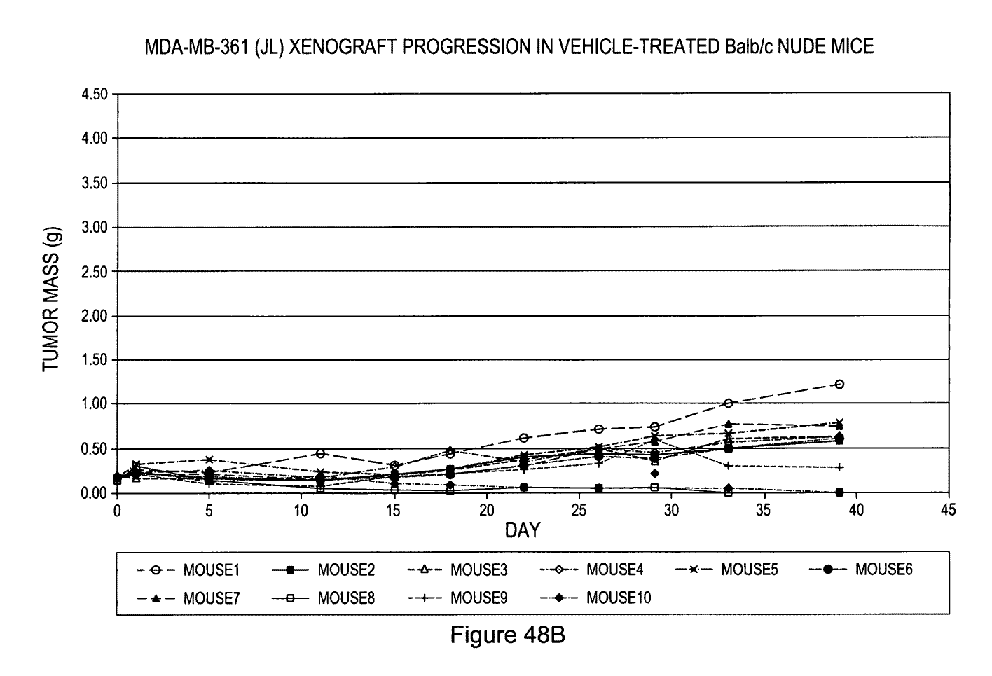

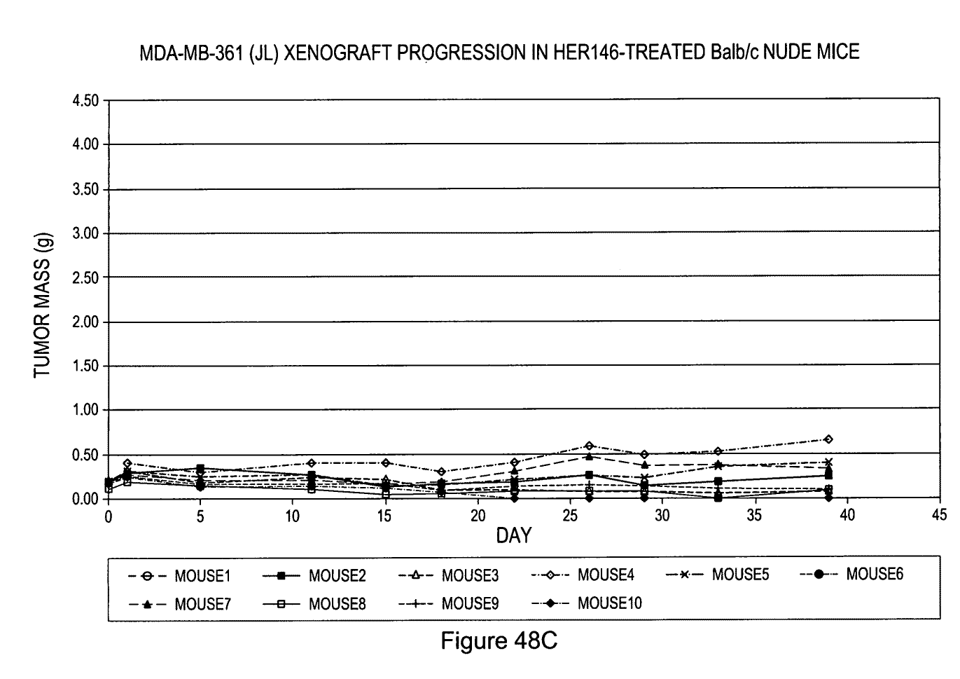

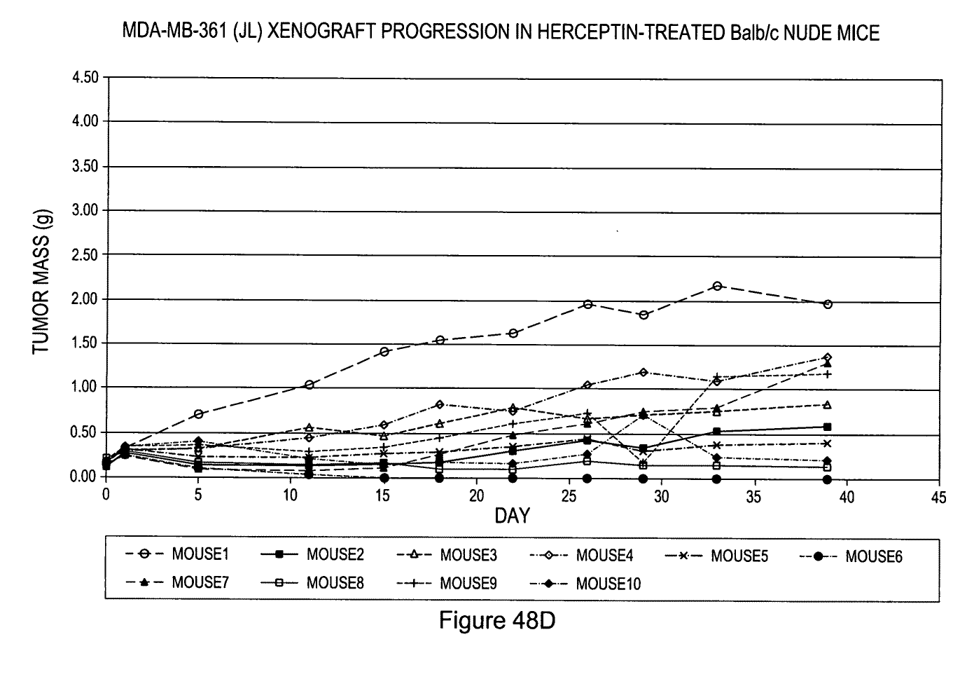

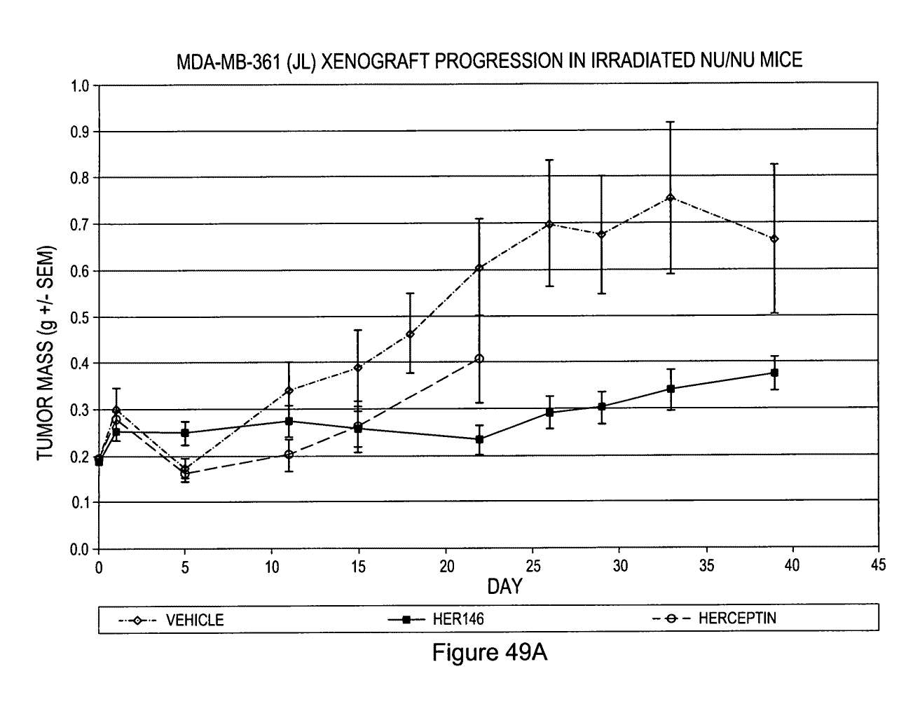

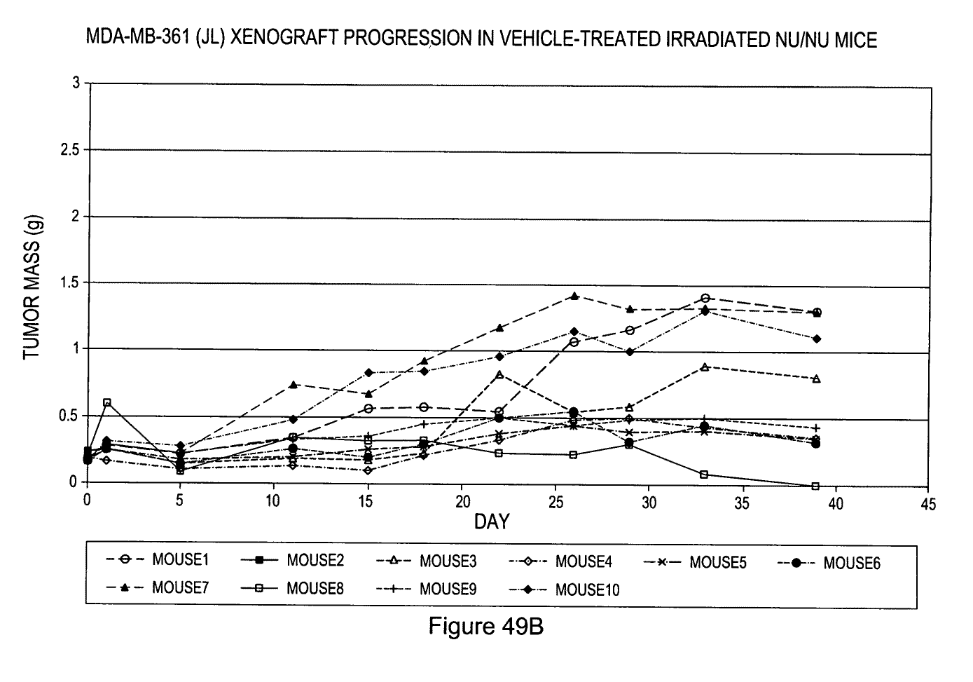

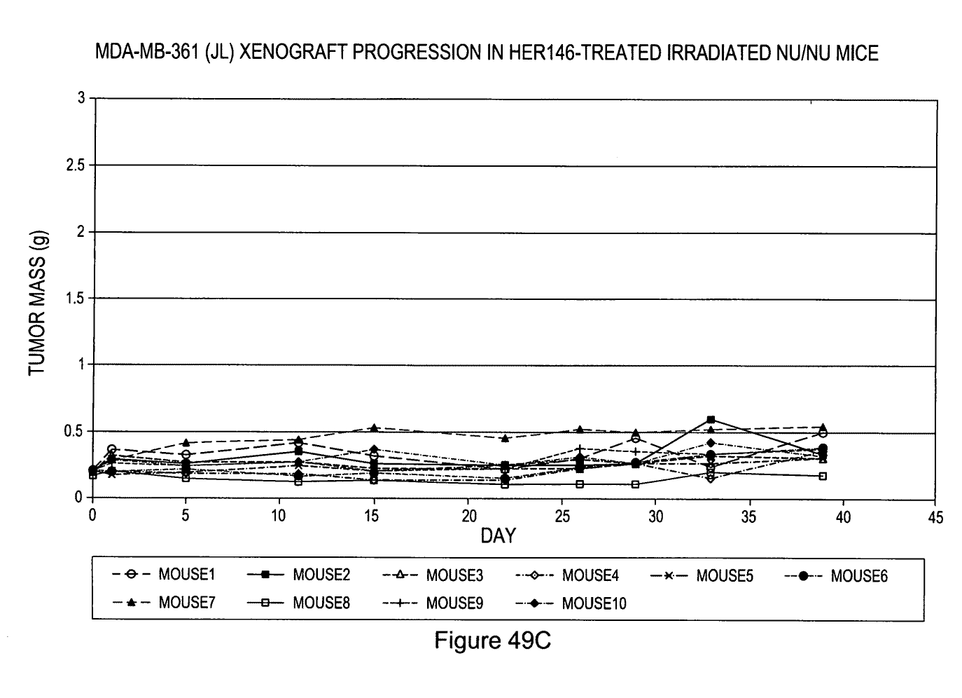

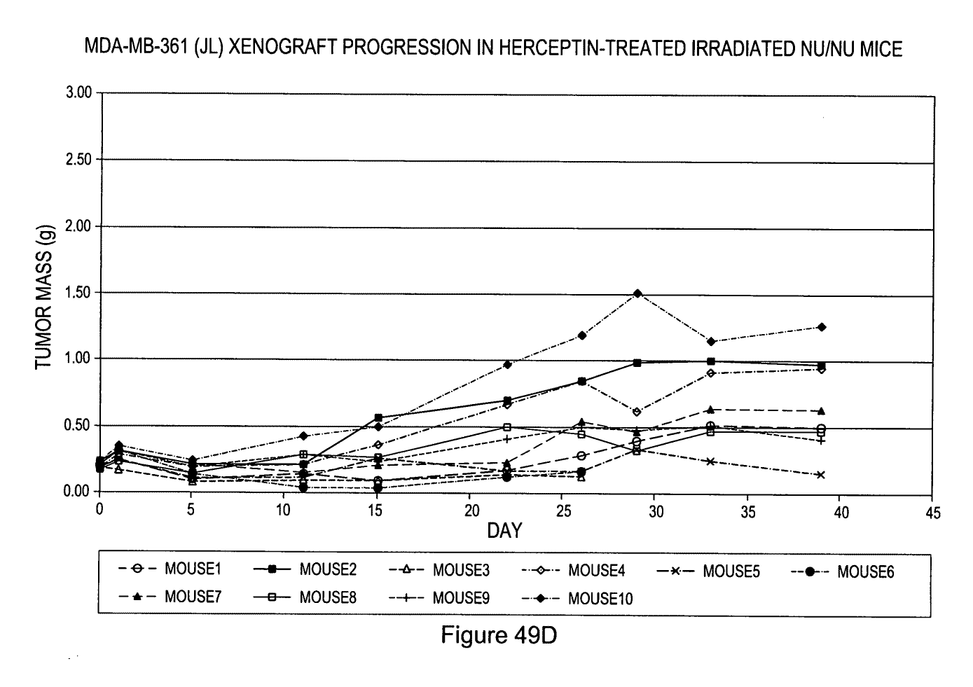

Female, athymic nude mice were exposed to total body irradiation (400 rads) to further suppress their residual immune system and facilitate the establishment of xenografts. Three days later, the irradiated mice were injected subcutaneously (SC) with 1×10⁷ MDA-MB-361 cells in Matrigel (Collaborative Biomedical Products, Belford, Mass., diluted 1:1 in culture medium) in the dorsal, right flank. When the tumors reached the mass of 0.1 to 0.25 g, the tumors were staged to ensure uniformity of the treatment groups. Male, athymic Balb/c nude mice were injected s.c. with 1×10⁷ cells in the right flank. When tumors reached an average tumor mass of 0.1 to 0.25 g, the tumors were staged to ensure uniformity of the treatment groups. Mice were dosed with compounds (100 μg/mouse ip) on days 1, 4, 6, 8 and 11 (n=10 mice/treatment group). All compounds were administered ip. Tumors were measured at least once a week and their mass (±SEM) was calculated. Tumor mass for each treatment group was compared to that from the vehicle-treated group for statistical significance using ANOVA and subsequent pairwise comparisons to the vehicle-treated group using a one-tailed t-test with the error term for the t-test based on the pooled variance across all treatment groups. The results are shown in FIGS. 19, 20, 48A-D and 49A-D.

The preliminary results in vivo as shown in FIGS. 46 and 47, however, indicate that HER116 was not efficacious against MDA-MB-361 (JL) xenografts in SCID-Beige mice and did not improve survival compared to negative control. These data correlate with other data herein that show that HER116 appears to lack in vitro anti-proliferation activity against MDA-MB-361 (JL) cells. HER146, on the other hand, was efficacious against MDA-MB-361 (JL) xenografts in SCID-Beige mice but demonstrated slower tumor regression than the positive control. Despite slower tumor regression, HER146 treated mice had better overall survival and tumor free progression than Herceptin (positive control) treated mice.

Based on the in vitro and in vivo results taken as a whole, the anti-ErbB2 binding proteins are believed to be efficacious in treating tumors.

### Example 11

### Identification and Screening of Antibodies that Bind to the Membrane Proximal Region of Her2/ERBB2

Ligand binding triggers ERBB2 dimerization and the activation of the intracellular kinase domain of ERBB2. Autophosphorylation of C-terminal tyrosines triggers the recruitment to these sites of intracellular signal transducers that regulate cellular processes such as proliferation, differentiation, motility, adhesion, protection from apoptosis, and transformation.

ERBB2 is frequently over-expressed in breast cancer. The existence of high levels of circulating soluble ERBB2 extracellular domain is associated with poor prognosis and decreased responsiveness to chemotherapy and endocrine therapy. In cell cultures, it has been shown that soluble ERBB2 extracellular domain arises by proteolytic cleavage of the extracellular domain of ERBB2. The cleavage of the extracellular domain results in a truncated, cell-associated, ERBB2 fragment that contains the intracellular kinase domain and a potentially surface-exposed N-terminal membrane proximal sequence, EQRASPLTSIIS (amino acid residues 645-656 of HER2). This membrane-bound fragment (designated as ERBB2 p95 because of its molecular weight) shows potentially enhanced signalling activity. It has been speculated that the adverse prognosis observed in patients with high levels of ECD/ERBB2 may be related, at least in part, to an increase of truncated, signalling-competent, ERBB2 p95.

Because the N-terminal membrane proximal sequence, EQRASPLTSIIS (referred here as the “stumpy” region) potentially remains on cell surface after the proteolytic cleavage of the extracellular domain, the stumpy region is a potential target for therapeutic intervention. For example, Herceptin® (Trastuzumab), as part of a treatment regimen containing doxorubicin, cyclophosphamide, and paclitaxel, is indicated for the adjuvant treatment of patients with ERBB2-overexpressing, node-positive breast cancer. However, Herceptin does not bind to the stumpy region of ERBB2. In contrast, an antibody that bind to the stumpy region of ERBB2 would be a more potent and effective inhibitor of the truncated, signalling-competent, ERBB2 p95.

Selection of scFvs that Bind to the “Stumpy” Region by Phage Display

Single chain fragment variable (scFv) moieties that bind to the membrane-proximal region of Her2 (ErbB2) that remains on the cell surface following cleavage and release of the soluble extra-cellular domain were identified following three rounds of selection using the Cambridge Antibody Technology (CAT) phage display libraries. Selection strategies are outlined in FIG. 5. Three CAT libraries were used; the Bone Marrow Vaughan (BMV) library (Vaughan et al, 1996), the combined spleen (CS) library and the DP47 library (unpublished). The “stumpy peptide” and “scrambled peptide” (FIG. 5A), each expressed with a biotin tag, were used during the selection and these peptides along with two Her2-Fc fusion proteins were used during the subsequent screening steps (see Table 3). The EKK sequence at the C termini of the stumpy peptide and scrambled peptide is predicted to maintain the helical structure predicted from the NMR (see, Goetz et al., 2001. Biochemistry 40: 6534-6540). For selections, aliquots of phage and magnetic streptavidin beads (Dynabeads M-280 streptavidin) were blocked separately in 3% milk/PBS for 1 hour at room temperature in a rotary mixer (20 rpm). Blocked phage were incubated with a 100 nM concentration of the scrambled de-selection peptide in round 1 (the amount of de-selection peptide decreased in subsequent rounds as the concentration of the selection peptide decreased), incubated at room temperature for 1 hour on a rotary shaker (20 rpm), mixed with blocked magnetic beads and incubated for a further hour. The de-selected library was collected by pelleting the beads using a magnetic separator. Biotinylated selection peptide (at various concentrations as indicated in FIG. 5A) was incubated with the de-selected phage library for 2 hours at room temp on a rotary mixer (20 rpm) followed by a 15 minute incubation with pre-blocked magnetic beads. Beads were separated using a magnetic separator and washed 10 times with PBS/0.1% Tween 20 and 3 times with PBS. Bound phage were eluted by incubation with a 10 ug/ml solution of trypsin in PBS for 30 minutes at 37° C. (100 rpm) followed by separation from the magnetic beads.

Eluted phage were used to infect 10 ml of an E. coli TG1 culture that had been grown to mid-logarithmic phase (corresponding to an OD₆₀₀ of ˜0.5). Bacteria were infected with phage for 1 hour at 37° C. with shaking at 150 rpm, concentrated following a centrifugation step and plated on 2×TY agar bioassay plates containing 2% glucose and 100 ug/ml ampicillin (2×TYAG). Various dilutions of E. coli culture infected with either input or output phage were also plated on 2×TYAG agar to determine phage titers. Following overnight growth at 30° C., 10 ml of 2×TYAG medium was added to each bioassay plate and the cells were re-suspended by scraping the bacterial lawn. Glycerol was added to this cell suspension to give a final concentration of 17% and stored in aliquots at −80° C. until further use. In order to rescue phage for the next round of selection, 100 μl of this cell suspension was used to inoculate 20 ml 2×TYAG medium, which was grown at 37° C. (300 rpm) to an OD₆₀₀ of 0.3-0.5. Cells were then super-infected with 3.3 μl of MK13K07 helper phage and incubated at 37° C. (150 rpm) for 1 hour. The cells were then centrifuged and the pellet re-suspended in a kanamycin/non-glucose containing medium (2×TY with 50 μg/ml kanamycin and 100 ug/ml ampicillin). This culture was grown overnight at 30° C. (300 rpm). Phage were harvested in the supernatant following centrifugation and were ready to use in the next round of selection as described above.

### Example 12

### ELISA to Measure Binding of scFvs Expressed in the Periplasm or Purified to Biotinylated Her2 Protein Constructs

A streptavidin-coated 96 well plate (Greiner) was washed three times with PBS/0.05% Tween 20 and blocked for 1 hour at room temperature in 3% skim milk/1% BSA/PBS. Plates were washed three times with PBS/0.05% prior to the addition of a 1 mg/ml solution of biotinylated Her2-Fc proteins (Her008P, Her017P, Her018P, Her054P) or a biotinylated negative control murine IgG2a protein. Plates were incubated for one hour at room temperature. Peripreps were prepared as described in an earlier section and were blocked for 1 hour at room temperature in an equal volume of 6% skim milk/1% BSA/PBS. Blocked plates were washed five times with PBS/0.05% Tween 20 and 50 ml/well of blocked periprep (or purified scFv diluted in block buffer) were transferred to the appropriate plates and incubated for 1 hour at room temperature. A 1 ug/ml solution of Herceptin (in blocking buffer) was added to well H12 of each plate to serve as a positive control. Plates were washed five times with PBS/0.05% Tween 20 prior to the addition of a 1:250 dilution of anti-myc peroxidase (Roche) or a 1:5000 dilution of goat anti-human peroxidase (Southern Biotech) secondary antibody to detect bound scFv or Herceptin respectively. Plates were incubated for a further hour at room temperature and washed seven times with PBS/0.05% Tween 20. Signal was developed using TMB, the reaction stopped with H2SO4 and the absorbance read at 450 nm on an Envision plate reader (Perkin Elmer).

### Example 13

### Conversion of scFᵥ to IgG

Heavy and light chain V regions from scFv clones are amplified with clone-specific primers. PCR products are digested with appropriate restriction enzymes and subcloned into vectors containing human IgG1 heavy chain constant domain (for V_{H} domains) or vectors containing human lambda or kappa light chain constant domains as appropriate (V_{L} domains). The closest human germlines of the V_{H} and V_{L} segments are determined and this information is used to indicate whether kappa or lambda light chain constant domains are used. Correct insertion of V region domains into plasmids is verified by sequencing of plasmid DNA from individual E. coli colonies. Plasmids are prepared from E. coli cultures by standard techniques and heavy and light chain constructs are co-transfected into COS cells using standard techniques. Secreted IgG is purified using protein A sepharose (Pharmacia) and buffer exchanged into PBS.

### Example 14

### Effect of SMIPS on HER2Surface Expression and Ectodomain Shedding

To investigate the effect of SMIPs on Her2 surface expression and ectodomain shedding, 200,000 SKBR3 cells/well were plated in 24-well tissue culture plates and incubated overnight. The next day, the media was carefully removed and the cells were incubated for 24 hours with fresh media containing 10 ug/ml SMIPs (Her067, Her094, Her102, Her116, Her146 or Her018) or molar equivalent of antibody (HERCEPTIN® as a positive control, or Retuxan as a negative control). The table below shows the SMIPs and antibodies that were used. As additional controls, cells were also treated with either 1 nM pervanedate to increase ectodomain shedding, or 5 ug/ml TIMP1a protease inhibitor that results in blockage of Her2 cleavage.

| SMIP/antibody (lot#, concentration) | |
|---|---|
| HER067 (InVivo 1566JK93 4.57 ug/ul) | HER033 |
| HER094 (InVivo 1714MM20 5.27 ug/ul) | HER030 |
| HER102 (InVivo 1681JK50 2.99 ug/ul) | |
| HER116 (InVivo 1714MM16 3.33 ug/ul) | |
| HER146 (InVivo 1714MM18 3.62 ug/ul) | |
| HER018 (1464JK49 5.75 ug/ul) | 4D5 SMIP |
| Herceptin (N42442 1688RAC33 2 ug/ul) | (+) Control for blocking |
| | cleavage |
| Rituxan (N36493 2 ug/ul) | (−) Control i.e. no |
| | binding/effect |
| Pervanedate (1 mM) | Increases ectodomain |
| | shedding |
| TIMP1 (5 ug/ml) | Block Her2 cleavage |

The levels of shed Her2 ECD in the supernatant was determined by ELISA. After 24 hours supernatants were harvested and the amount of shed Her2 ectodomain determined by ELISA, using HERCEPTIN® to capture shed ectodomain and anti-Her2 TA-1 antibody to detect the captured ectodomain. Cells were harvested using trypsin and cell surface Her2 was determined by flow cytometry by staining with the SMIP or antibody used for the treatment. Levels of Her2 were determined and compared to untreated cells stained with the same SMIP or antibody.

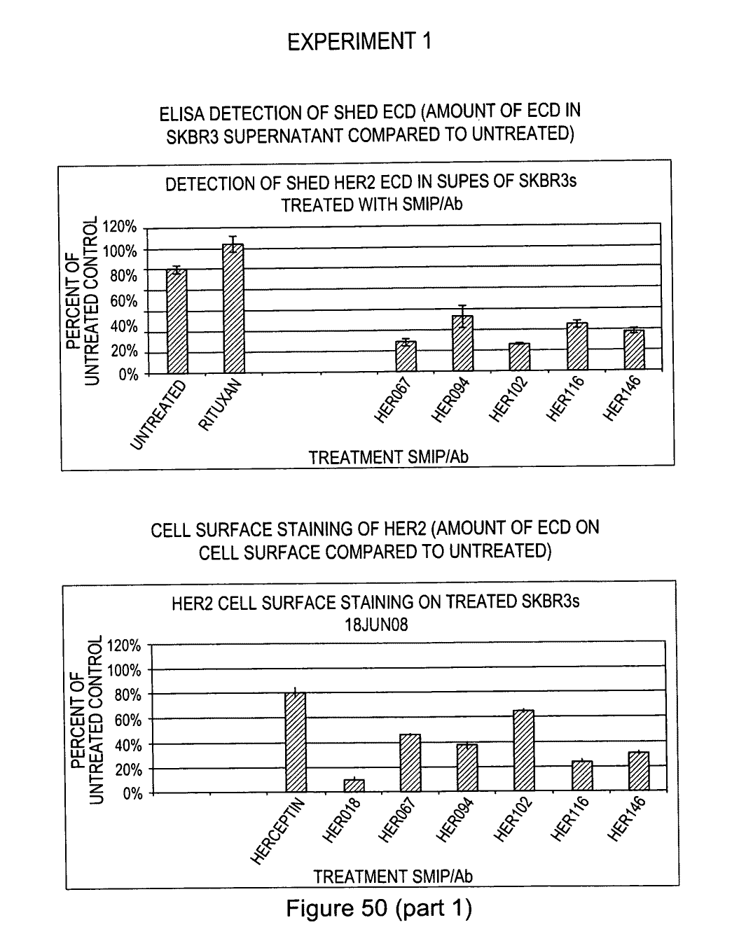

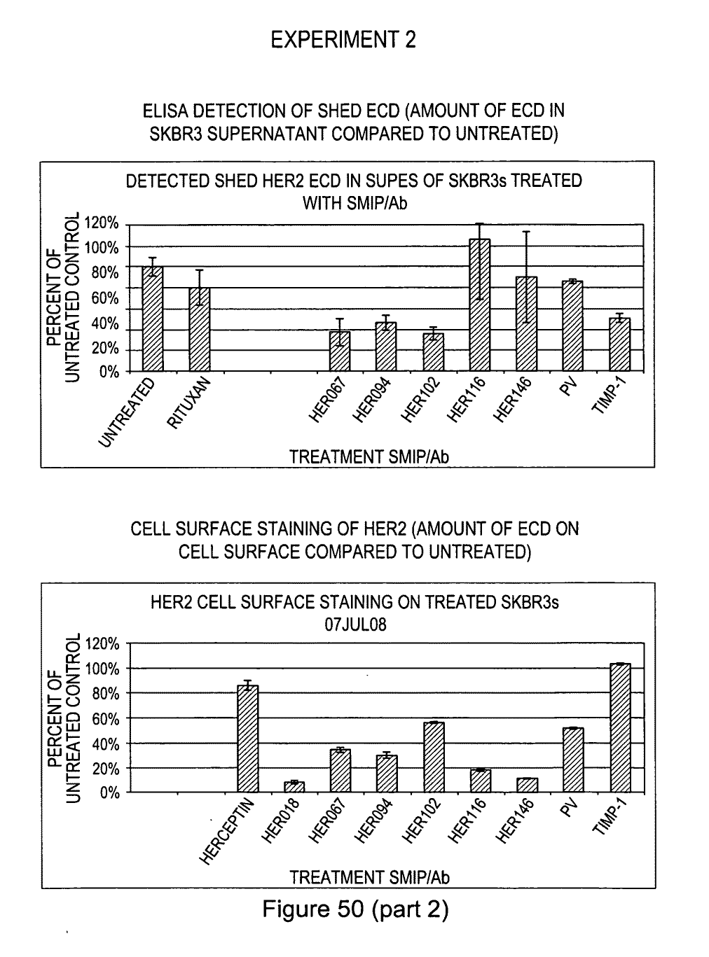

HERCEPTIN® treated cells were not detected by ELISA. As shown in FIGS. 50A and 50B, SMIPs decrease shedding of the Her2 ectodomain. As shown in FIGS. 50C and 50D, anti-HER2SMIPs of the invention decrease cell surface Her2.

Without being bound by theory, the mechanism for SMIPs' decreasing cell surface Her2 and shedding Her2 ectodomain may be that the SMIP blocks Her2 cleavage, thus reducing shed ectodomain and production of p95 Her2. Alternatively, SMIPs could increase Her2 internalization, thus reducing cell surface ECD. Similar mechanisms have been described for HERCEPTIN®.

### Example 15

### Anti-HER2SMIP Cross-Blocking

To investigate the ability of Her2 SMIPs and antibodies to block each other's binding to cell surface Her2, cross-blocking was investigated using FMAT blocking buffer. SMIPs were labeled with FMAT Blue as per manufacturers directions (Applied Biosystems). Unlabeled competitor SMIPs or Antibodies were diluted to 400 nM in FMAT Blocking Buffer (44 ug/mL for SMIPs; 59.2 ug/mL for antibodies). Each protein was titrated 1:3 in FMAt blocking buffer in duplicate V-bottom tissue culture 96-well plates in a final volume of 60 ul/well. Cells (SKBR3) were added in 60 ul FMAT blocking buffer to give 36,000 cells/well. Plates were incubated for 1 hour at room temperature before adding FMAT Blue labeled antibodies at a concentration determined to give maximal staining in the absence of competing unlabeled SMIP or antibody (5 ug/mL for HERCEPTIN®; 2 ug/mL for HER018, 10 ug/mL for all other HER SMIPs, and 2 ug/ml for Rituxan and 2LM20-4 (anti-CD20 SMIP)). Plates were incubated at room temperateure for 45-60 minutes (10 minutes for Herceptin). Cells were spun down at 1250 rpm for 5 minutes and non-bound SMIPs and antibodies flicked off. Cells were resuspended in 120 ul FMAT Blocking Buffer and transferred to FMAT 96-well plates. Cells were allowed to settle for 5 minutes before being read on FL1 AB3200. The average of each duplicate value of FMAT staining was determined for each concentration of competing unlabeled protein.

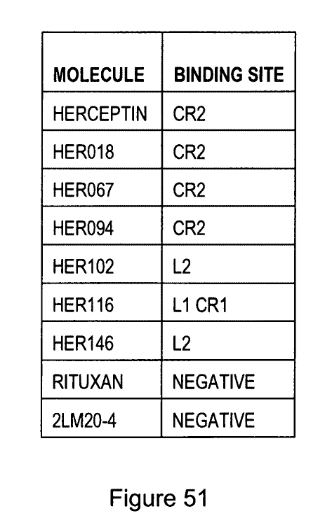

Cross-blocking between Her2 SMIPs and antibodies is not necessarily indicated by epitope mapping. SMIPs and antibodies that bind different epitopes could nevertheless block each other due to binding stoichiometry and molecular size. FIG. 51 shows a summary of the Her2 binding site possibilities for various SMIPs.

HERCEPTIN® binding is blocked by HERCEPTIN® and HER018 at low concentrations; HER067, HER102, HER146 at higher concentrations; and HER094 at very high concentrations. HER018 binding is blocked by HER018 and HERCEPTIN® at low concentrations; HER067, HER102, HER146 at higher concentrations; and HER094 at very high concentrations. HER067 binding is blocked by HERCEPTIN® and HER018 at low concentrations; HER067 and HER102 at higher concentrations; and HER094 and HER146 at very high concentrations. Also, HER067 binding is greatly enhanced by HER116 binding.

HER094 binding is blocked by HERCEPTIN® and HER018 at low concentrations; HER067 and HER102 at higher concentrations; and HER094 and HER146 at very high concentrations. Also, HER094 binding is greatly enhanced by HER116 binding.

HER102 binding is blocked by HERCEPTIN® and HER018 at low concentrations; and HER146 and HER102 at higher concentrations. Also, HER102 binding may be slightly enhanced with HER116 binding.

HER116 binding is blocked by HER116 at low concentrations. No other SMIPs or antibodies blocked HER116 binding. HER146 binding is blocked by HERCEPTIN®, HER018 and HER102 at low concentrations; and HER146 at higher concentrations. Anti-CD20 Ab and SMIP binding is not blocked by any HER2SMIPs or antibodies.

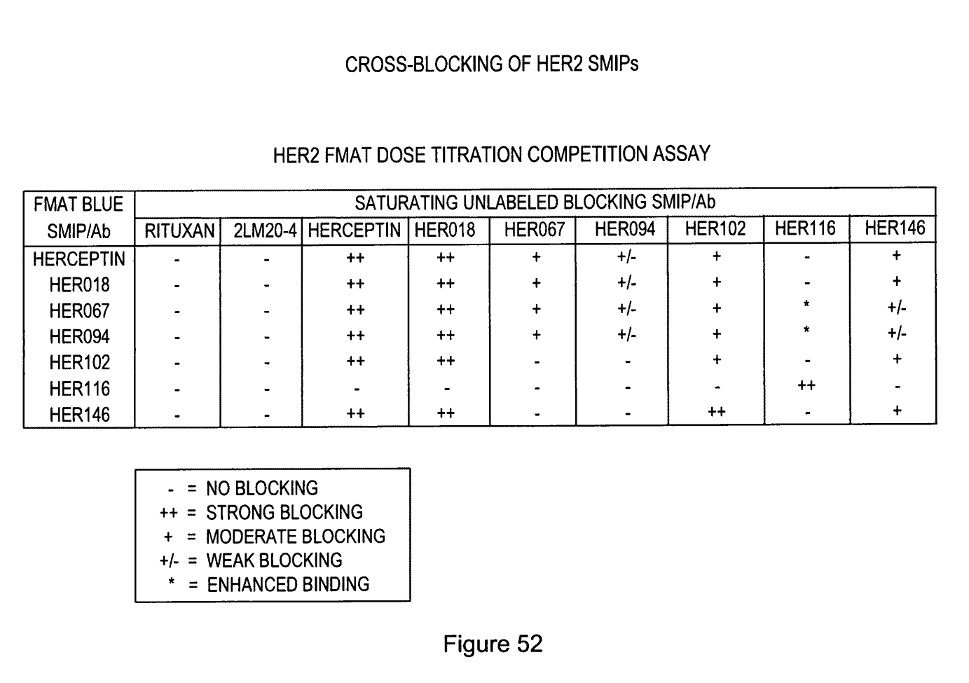

The SMIP cross-blocking results are summarized in FIG. 52.

Importantly, Her2 SMIPs that do not cross-block each other have the potential to simultaneously bind to a Her2 molecule. Accordingly, there may be an additive mechanism of action for Her2 binding with SMIPs and antibodies. Further, there is a possibility for a combination treatment with multiple SMIPs or with a combination of SMIP and antibody. SMIPs could also be potential partners for bispecific molecules such as Scorpions™.

### Example 16

### Anti-HER2SMIP Internalization Assay

We investigated the ability of anti-HER2SMIPS of the invention to internalize in various cell lines as follows.

Hum-ZAP Internalization Assay

Hum-ZAP (Advanced targeting Systems) is a saporin-conjugated anti-human Ig that targets and eliminates cells using the internalization of an antibody or SMIP. Upon binding to a human IgG containing molecule, such as a SMIP or antibody, that recognizes an extracellular domain of a cell surface antigen, Hum-ZAP is taken inside the cell by antibody or SMIP-mediated internalization. The entrance of saporin into the cell will result in protein synthesis inhibition and eventual cell death after 2-4 days.

Cells in 90 μl of media (at a concentration of 2.5−5.0×10³ cells/well) were added to 96-well plates and incubated overnight. The following day, cells were treated by either: a) the addition of 5 μl of a SMIP and media; b) the addition of 5 μl goat IgG-SAP (goat anti-human IgG negative control) and media; or c) the addition of 5 μl of a SMIP and Hum-ZAP (Saporin-conjugated goat anti-human IgG). Cells were incubated a further 96 hours before being assayed for proliferation using standard BrdU-incorporation and Hoechst nuclear staining. Internalization was observed as a reduction in cell proliferation an plotted as percent of untreated control.

We observed that, of the SMIPs tested, HER116 was best internalized by cells—these results agree with the fluorescence internalization experiments with “stumpy” binders.

Stumpy binders (HER156 and HER169) were observed to internalize in MDA-MB-361 JL, MDA-MB-453, and BT-474 cells and, to some degree, in SKBR3 cells. In addition, all SMIPs were internalized to some degree in BT474 and SKBR3 cells.

No detectable SMIP internalization, however, was observed in JIMT-1 and MDA-MB-361 (ATCC) cells. This may be due to the fact that MDA-MB-361 (ATCC) cells grow slowly. Thus, longer treatment times with increased cell numbers may be necessary in order to detect a response.

Fluorescence Assay

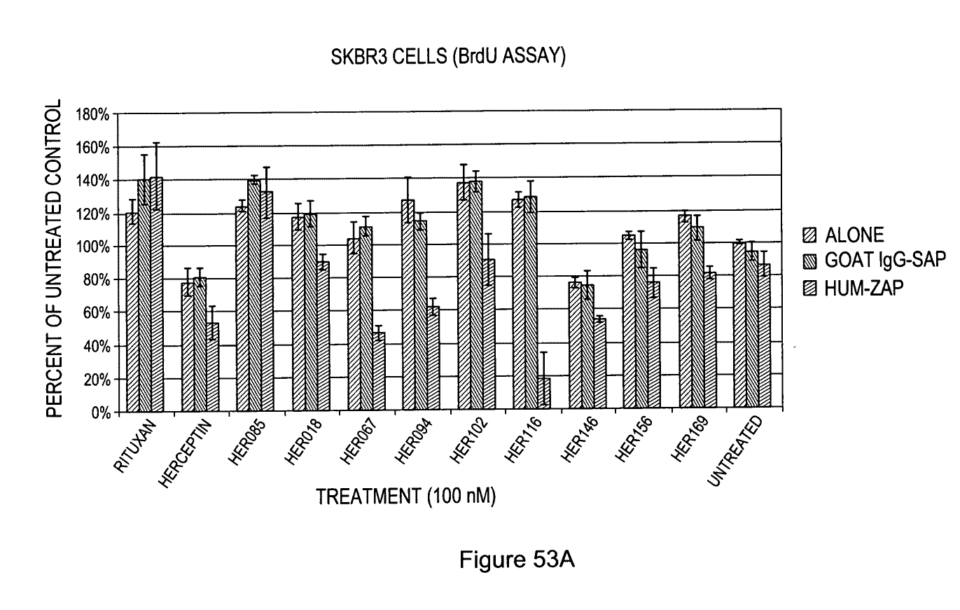

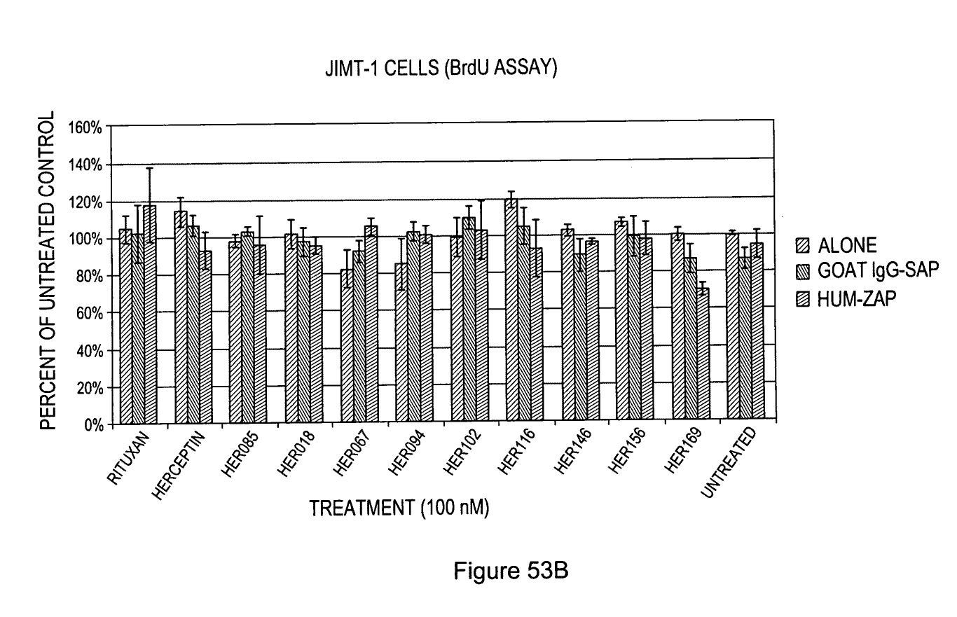

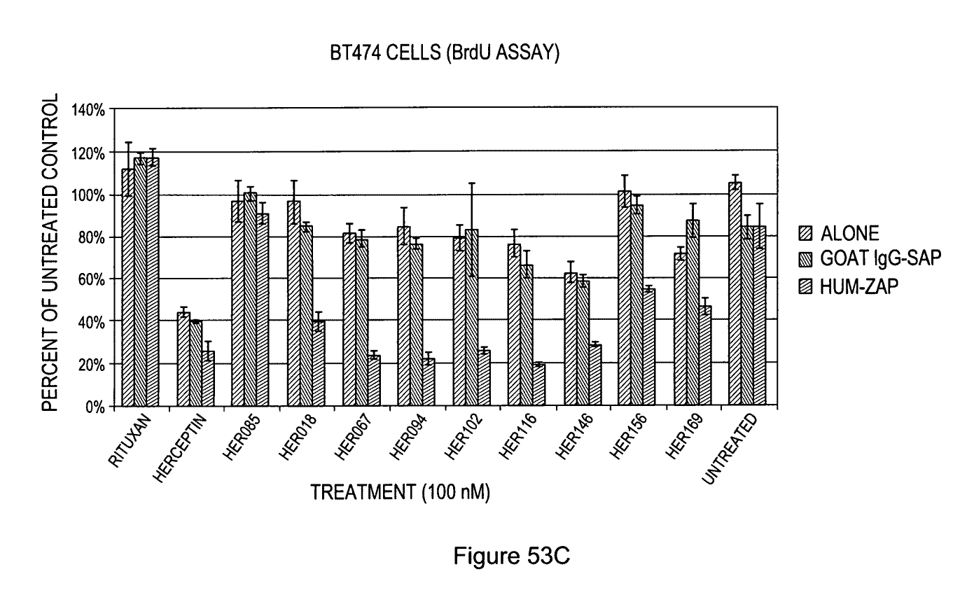

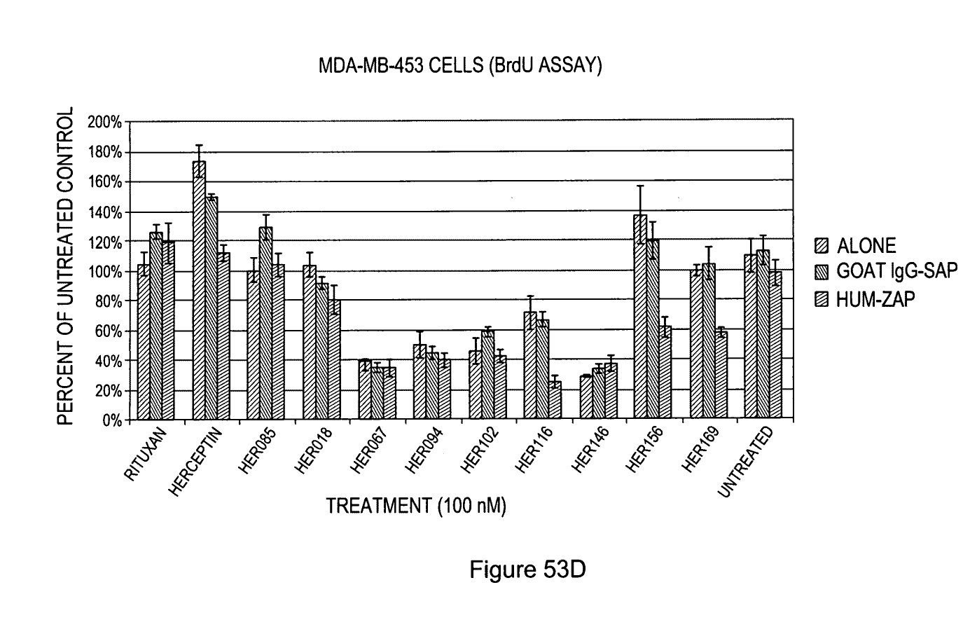

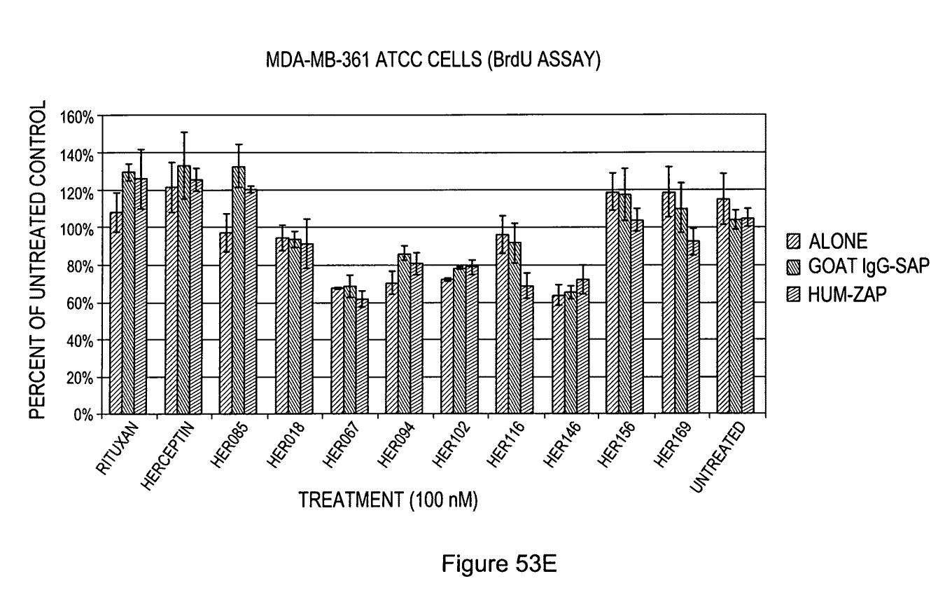

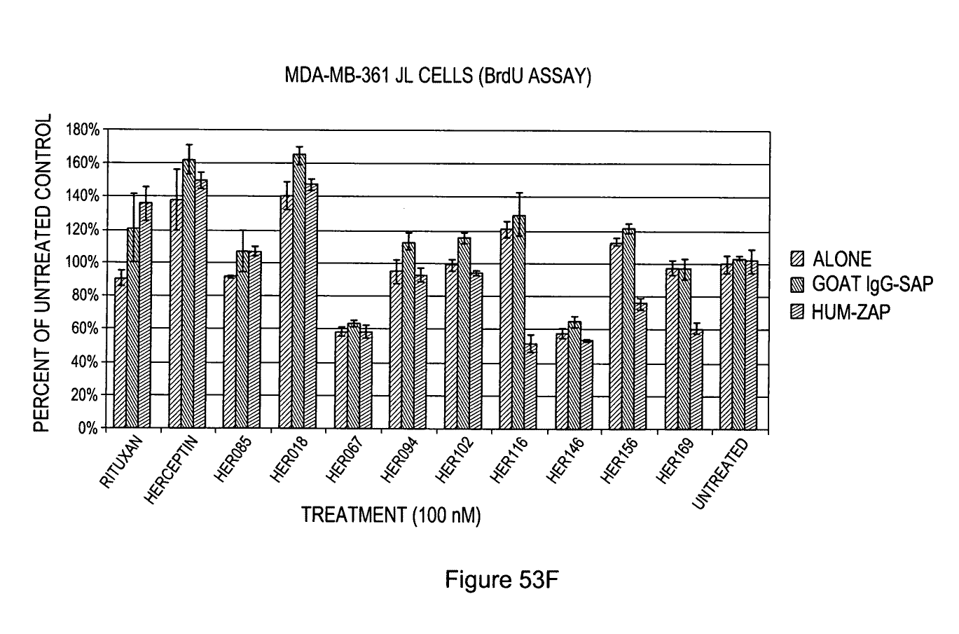

MDA-MB-361 cells were grown in 96-well plate format and treated with anti-Her2 SMIPs, Herceptin (Herc) or control anti-CD20 SMIP for indicated times. Media was removed and cells fixed (4% paraformaldehyde) and permeabilized (0.2% Triton100). Cell surface or intracellular SMIPs or monoclonal antibodies were detected by staining with FITC-labeled anti-hulgG-Fc (see FIG. 53A-F, panels A and B or with rabbit anti-Her2 mAB (Cell Signaling Technologies) with secondary FITC-labeled Goat-anti-Rabbit IgG (Molecular Probes) (panel C). Fluorescent image detection was visualized by Cellomics High Content assay.

Her116 demonstrated rapid binding and internalization of SMIP (Panel A: 10 min; Panel B: 1 hr) and cell surface Her2 (Panel C: 1 hr) similar to Herceptin mAB. In contrast, Her 46 treatment demonstrated slower kinetics of cell surface binding that was sustained for longer time periods (Panel B: 1 hr) and confirmed with anti-Her2 cell surface localization (Panel C: 1 hr). Control anti-CD20 SMIP did not display binding at any time point as anticipated.

cypHER5E Assay

SMIPs and antibodies were labeled with CypHer5E (GE Healthcare) as per manufacturers direction. CypHer5E has little or no fluorescence at physiological pH, but fluoresces at low pH (e.g., when internalized into lysosomal compartments). Cells were plated in serum-free media and placed on ice for 5-10 minutes. Cells were then washed (1×) with cold media containing 1% FBS. Dilutions of CypHer5E labeled SMIPs or antibodies in ice cold serum-free media were added to cells and incubated on ice for 45 minutes. Cells were washed (1×) with ice cold media containing 1% FBS. Room temperature media containing 1% FBS (without phenol red, pH 7.6) and Hoechst nuclear stain (Invitrogen) were added to cells and the. Cells were then imaged using an InCell analysis System (GE Healthcare) using the 20× objective at 10 or 30 minute intervals for 2-4 hours. Cells were then fixed with formaldehyde, permeabilized, blocked and stained with an anti-human Alex-488 secondary mAb (anti-human IgG H+L, Invitrogen) and re-imaged on the InCell to verify SMIP or antibody binding. CypHer5E is imaged in the red channel (650-700 nm), Hoechst in the blue channel (387-525 nm) and the Alex-488 secondary antibody in the green channel (485-525 nm).

HER018 and HER116 were rapidly internalized—within 10 minutes. We confirmed the presence of SMIP binding after fixation with an anti-human Fc secondary Ab. The presence of SMIP binding was confirmed after fixation with an anti-human Fc secondary Ab. We found that HER067, HER146, HER156, and HER169 were internalized more slowly. We observed some internalization of these SMIPs by 4 hours. We found that Herceptin was internalized at a faster rate in SMIP format (HER018) than as Ab. In general, we found that “stumpy binders” were internalized over a longer period of time. Some had internalization by 4 hours. Without wishing to be bound by any theory, we believed that they could induce cell death over a period of days through internalizing of a co-incubated toxin-conjugated anti human secondary antibody.

### Example 17

### Effector Functions of Anti-HER2SMIPS

SKBR3 cells were harvested with trypsin and washed. Cells were labeled with BADTA (Perkin Elmer) by incubating 2×10⁶ cells in 2 ml media with 20 μl BADTA mix (5 μl BADTA reagent, 2 μl PF127, 13 μl DMSO) for 20 minutes at 37 C. Labeled cells were washed with PBS (4×) and resuspended in media at 400,000 cells/ml. Cells (20,000 cells in 50 μl) were aliquoted into a V-bottom plate and 50 μl of 2×SMIP or antibody were added. To a 50 μl aliquot of loaded target cells, 150 μl media were added and immediately spun down; background spontaneous release counts were determined in the 100 μl supernatant. To a second 50 μl aliquot, 150 μl 2% NP40 in media were added to lyse cells in order to determine maximum lysis. Effector cells (PBMC: peripheral blood mononuclear cells) were added to samples and incubated for 2 hours at 37 C. Plates were centrifuged to pellet cells/debris. Cleared supernatant (20 μl) was transferred to a flat bottom plate to which 200 μl of Europium solution was added and incubated for 15 minutes at room temperature while shaking. Fluorescence was measured (excitation 335 nm, emission 615 nm, cutoff 590 nm, delay 250 μs, and integration 1250 μs).

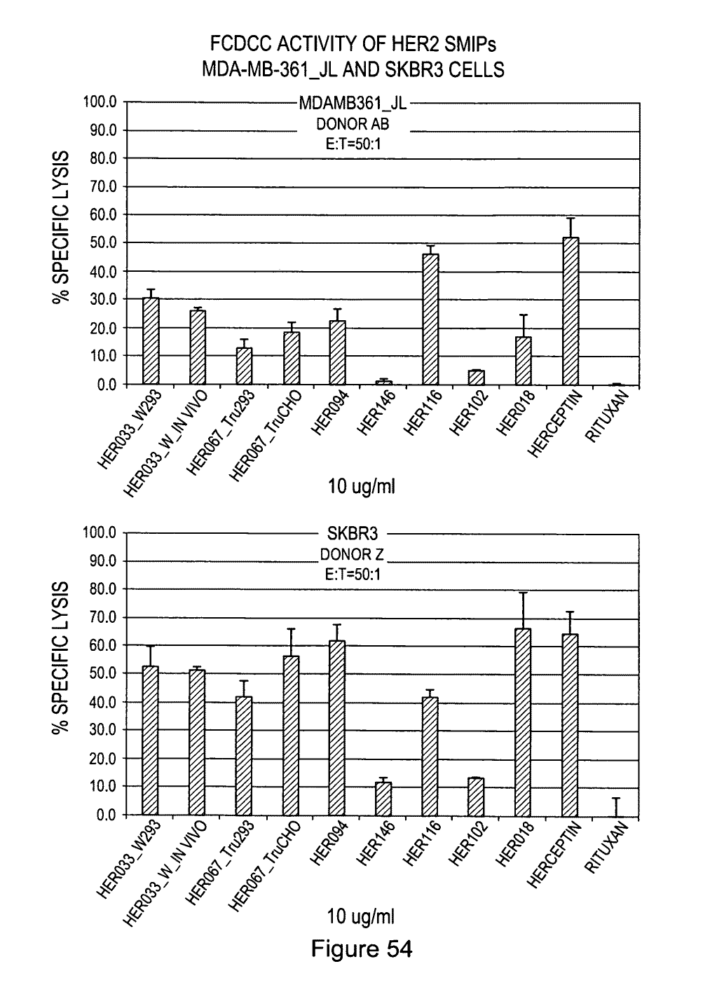

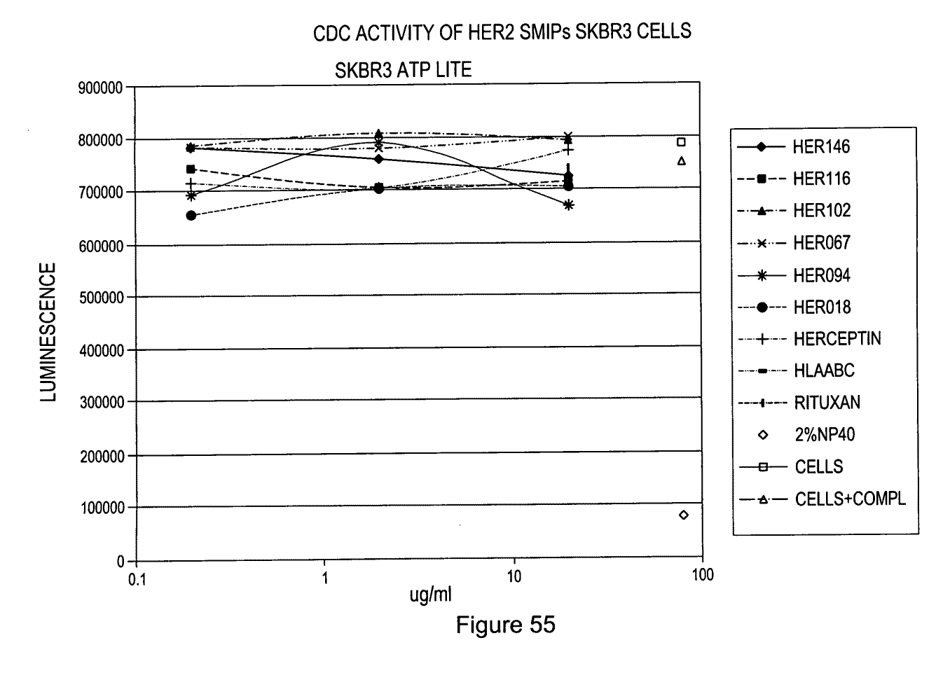

As shown in FIG. 54, HER116m HER033/067 and HER094 have good to moderate FcDCC activity that is comparable to that of HERCEPTIN® and HER018. In contrast, we observed no CDC activity with anti-HER2SMIPS, HERCEPTIN® or HLA ABC in SKBR3 cells (see FIG. 55) or MDS-MB-361-JL cells (data not shown).

### Example 18

### Serum Stability of Anti-HER2SMIPS

Stability of SMIPs in mouse plasma was determined by incubating SMIPs (200 ug/ml) in mouse plasma or PBS at 37 C or 4 C for up to 96 hours, with samples removed at intermediate times. A dilution series was made for each SMIP sample and the concentration was determined by ELISA using plates coated with a Her2 ECD murine Fc fusion protein (Her2SIIS::muFc). Captured SMIP was detected using a HRP-conjugated secondary anti mouse Fc secondary antibody. Mouse plasma alone or an anti-CD20 antibody, were used as negative controls in these experiments.

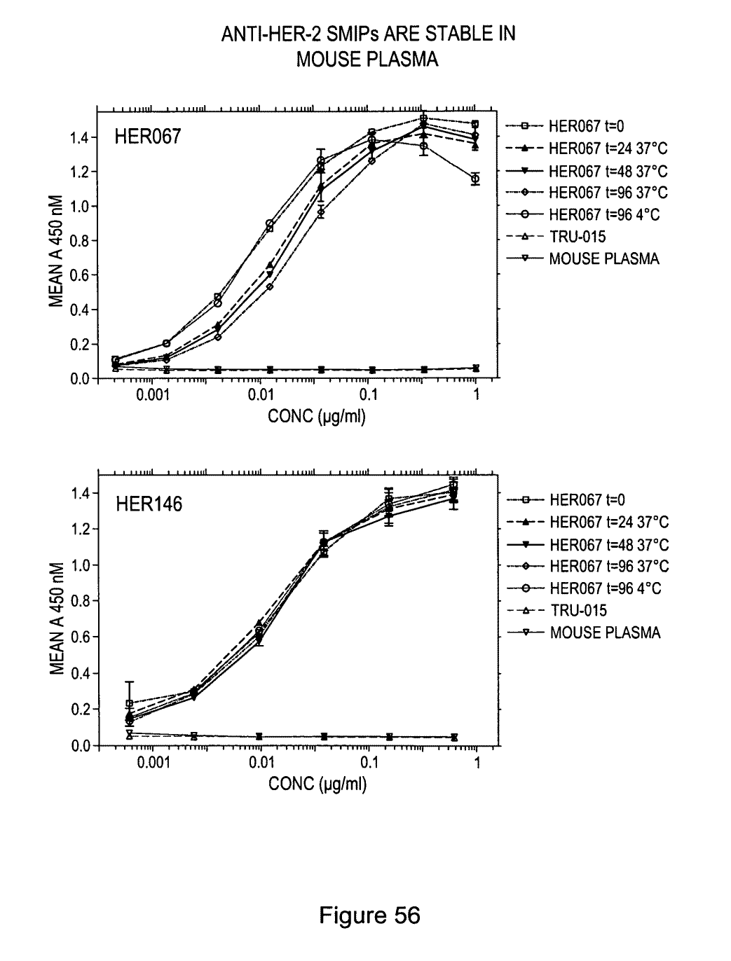

Results are shown in FIG. 56A. The effects of different incubation times and different temperatures had little effect on the binding of HER067 or HER 146 with Her2-SIIS at all of the concentrations tested. These results suggest that HER067 and HER146 are stable in plasma at physiologically-relevant temperatures and for extended periods of time. Repeat experiments that compared samples incubated in plasma with those that were incubated in PBS for 72 hr provided results that agreed with the first assay shown in (see FIG. 56B).

### Example 19

### Determination of Mass of Soluble HER2 Receptor-Binding Molecule Complex

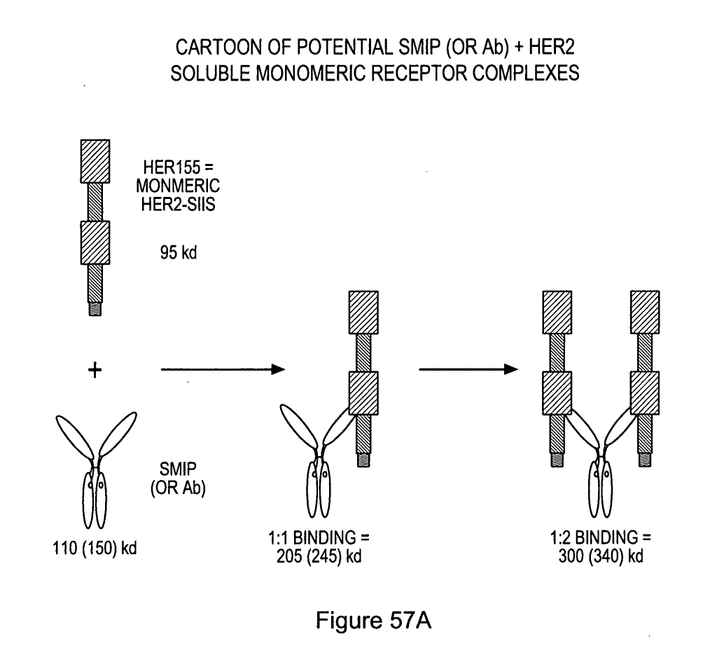

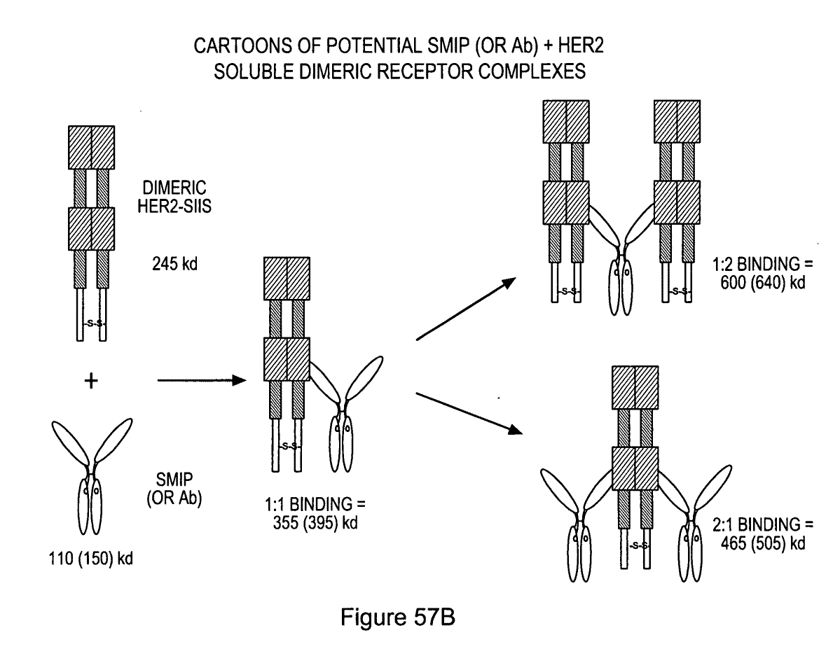

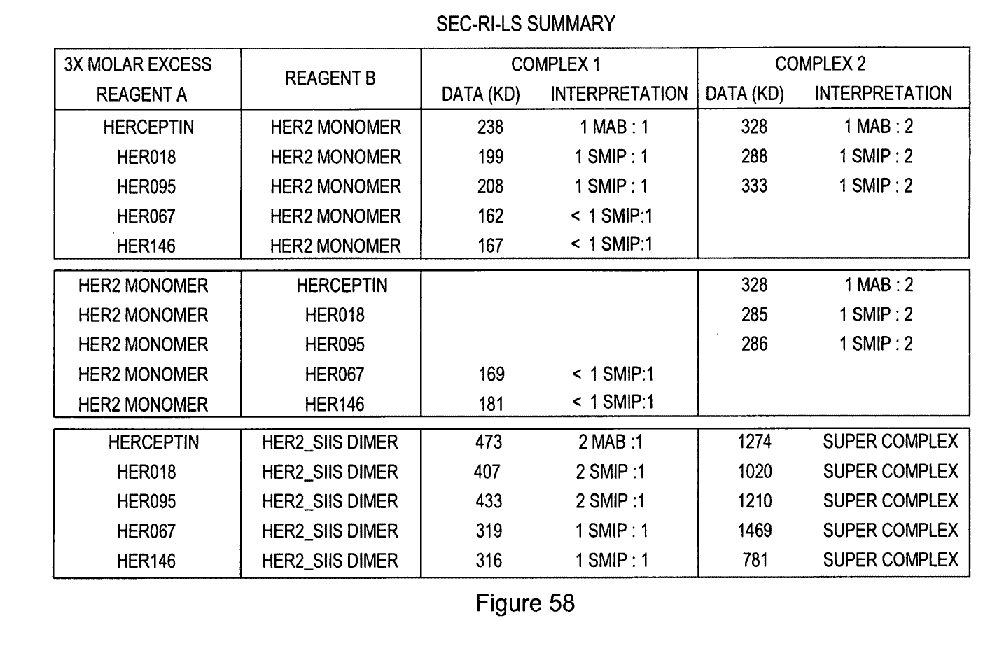

In order to determine the binding ratios of SMIPs to Her2 receptors, the mass of Her2 receptor/binding domain complexes was analyzed. FIGS. 57A and 57B show predicted masses of various SMIP/receptor complexes. SMIP (or mAb) was mixed with a soluble Her2 receptor at a 3:1 Molar ratio (an alternate mixture with a SMIP:receptor Molar ratio of 1:3 was also used), and the mixture was incubated at room temperature overnight. The mixture volumes were then normalized to 110 μl, and 100 μl of the SMIP/receptor mixture were subjected to size-exclusion chromatography combined with refractive index, multiple angle laser light scattering (SEC-RI-MALLS), using a TOSOH TSK G4000 SW_{XL} column. The mass of the resolved peaks was analyzed using ASTRA software (Wyatt Technology Corporation, CA). The results of the mass analysis are shown in FIG. 58.

Our observations suggest that each Herceptin, HER018, and HER095 molecule binds two monomeric soluble Her2 receptors simultaneously. This binding appeared to be complete with 2 Her2 molecules per SMIP when the receptor was in 3× excess. In contrast, it appears that HER067 and HER146 can only bind one monomeric soluble Her2 receptor. This binding appeared to be incomplete/poor with 0 to 1 Her2 molecules per SMIP when the receptor was in 3× excess. The data also support that two Herceptin, HER018 or HER095 molecules can bind to each dimeric soluble Her2 receptor, when the SMIP was in 3× excess. Alternatively, it appears that only one HER067 or HER146 molecule binds each dimeric soluble Her2 receptor, even when the SMIP was in 3× excess.

### Example 20

### Drug Combination Studies

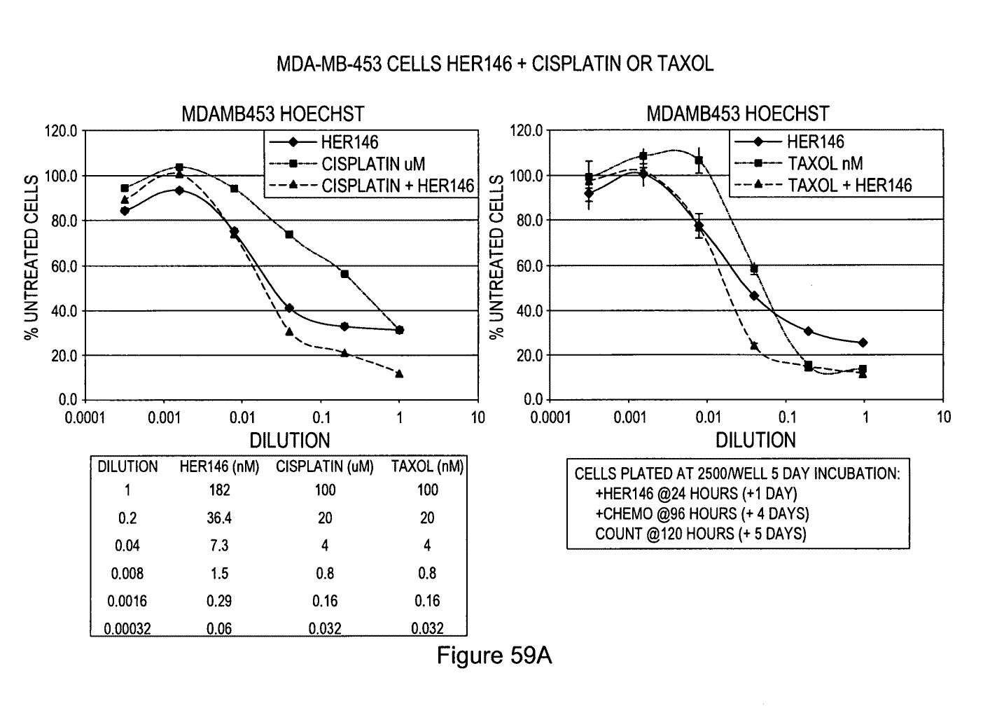

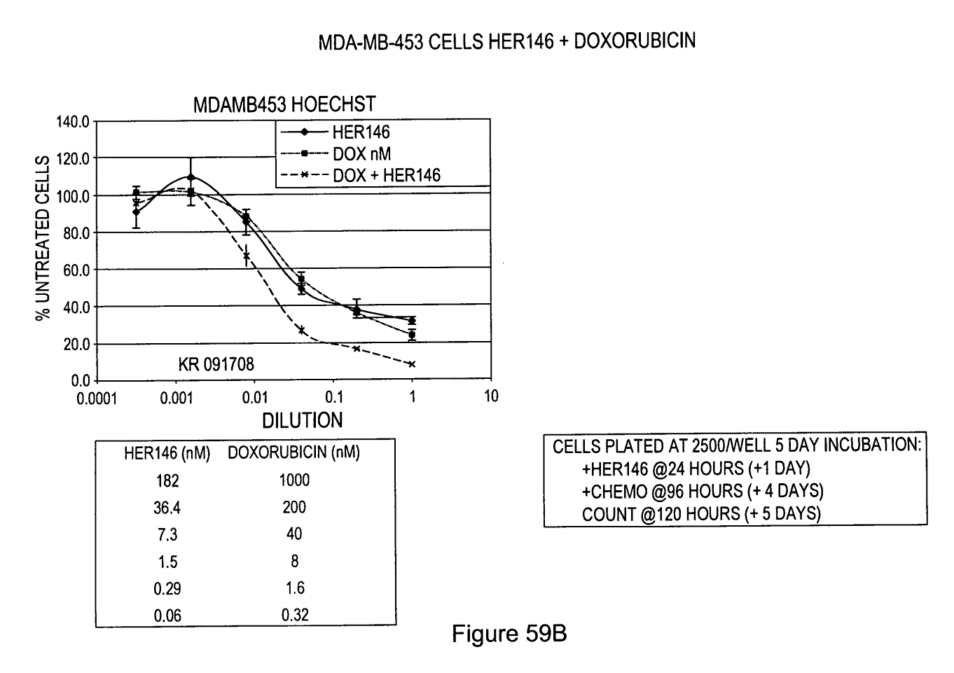

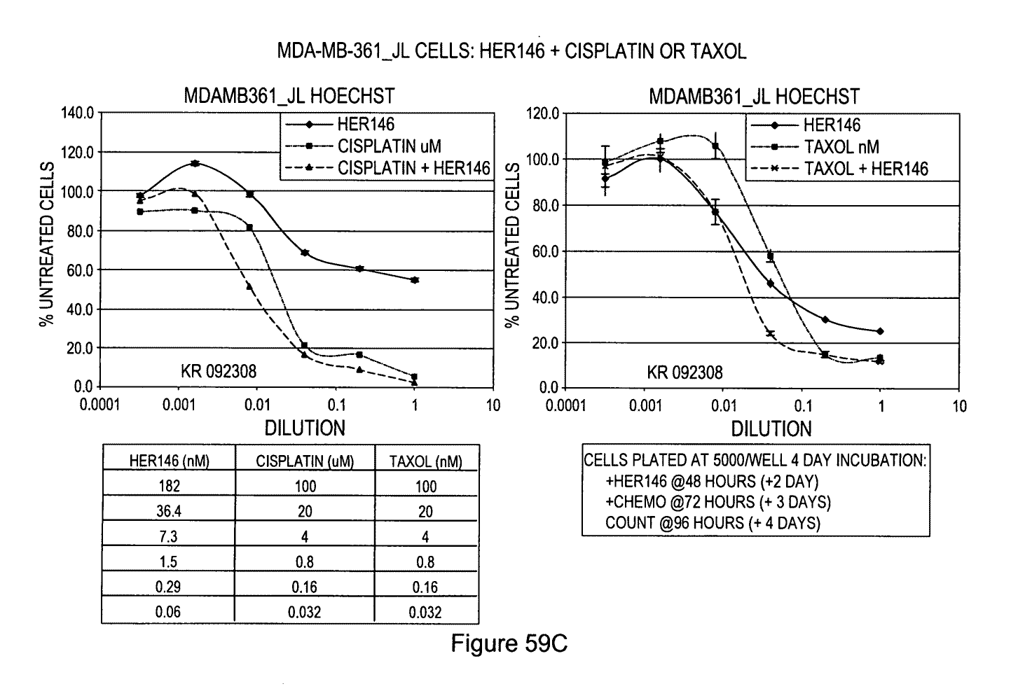

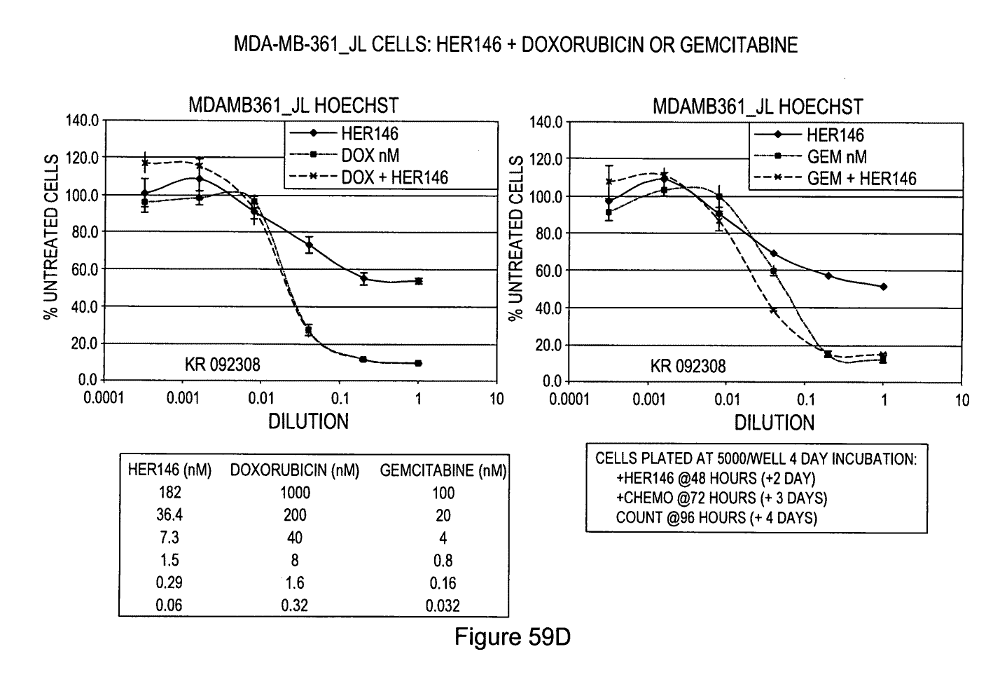

Cell cycle data suggested that MDA-MB-453 and MDA-MB-361_JL cells treated with Her146 and other SMIPs were driven into and through S-phase before being arrested in G1. The ability for SMIPs to sensitize cells to chemotherapeutic agents was determined by performing standard proliferation assays (BrdU incorporation and/or Hoechst nuclear staining) on cells treated with SMIP/antibody prior to treatment with chemotherapeutic agents (Cisplatin, Taxol, Doxorubicin or Gemcitabine). Cells (2500-5000 cells per 96 well) were treated with SMIP for 24 or 72 hours prior to the addition of chemotherapy. Cells were treated with the combination of SMIP/antibody and therapeutic an additional 24 hours before the cells were quantitated by counting cells using the nuclear stain, Hoechst, or by the ability of live cells to incorporate BrdU using standard assays. A 5-fold dilution series was run for each assay/treatment with a maximal concentration of SMIP of 182 nM and 100 uM Cisplatin, 100 nM Taxol, 1000 nM Doxorubicin, or 100 nM Gemcitabine with the ratio remaining constant for each dilution. The combination of SMIP and chemotherapy was compared to either SMIP or chemotherapy alone. Dose response curves of cells pre-treated with HER146 and then treated with various chemotherapeutic agents or combinations thereof are shown in FIG. 59A-D.

We found that Her2 SMIPs could have additive effects when administered with chemotherapeutic agents. For example, MDA-MB-453 cells treated with HER146 were more sensitive to chemotherapeutic agents (e.g., Cisplatin, Taxol, and Doxorubicin). MDA-MB-361-JL cells treated with Her146 were more sensitive to some chemotherapeutic agents (e.g., Cisplatin, Taxol, and Gemcitabine) but not others (e.g., Doxorubicin).

### Example 21

### Assessment of Target Binding Specificity

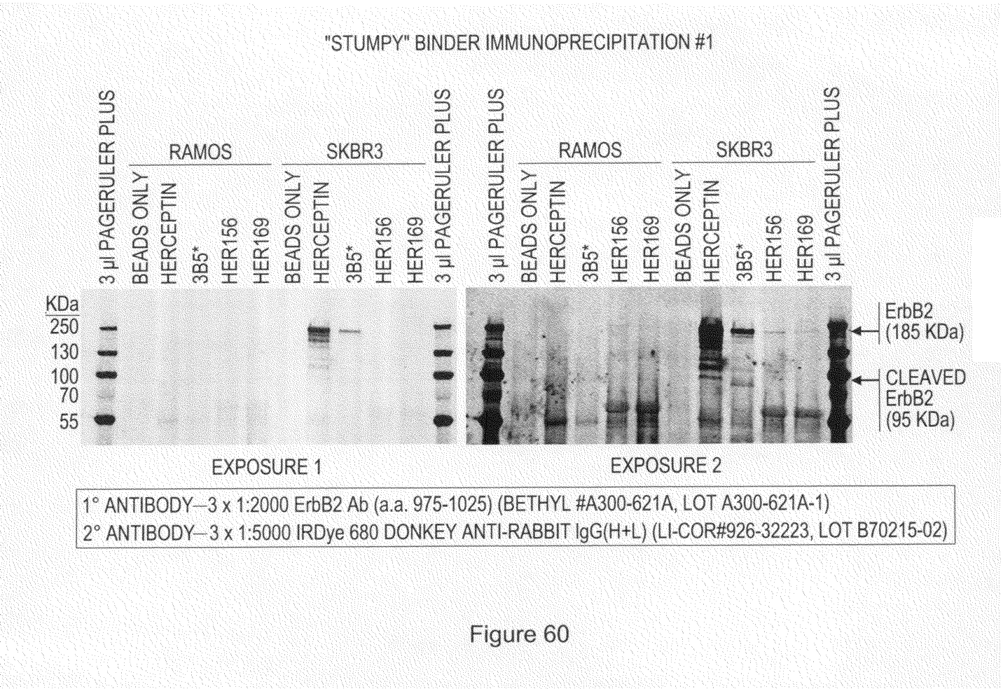

We investigated the target-binding specificity of the anti-HER2SMIPS referred to herein as “stumpy” binders by immunoprecipitation. Cells was solubilized in either Radio Immuno Precipitation Assay (RIPA) lysate buffer or Nonidet P-40 lysis buffer (a more gentle detergent than those in RIPA buffer) containing a cocktail of protease inhibitors. Cleared lysate protein concentrations were determined using a standard protein assay (e.g. Bradford assay). Her2 was immunoprecipitated from 1 mg RIPA lysate or 2 mg Nonidet P-40 lysate using 5 ug of SMIPs, 5 ug human IgG (as negative control) or 2 ug mouse monoclonal antibody, 3B5, against the intracellular region of Her2 (positive control). Immunoprecipitated protein is pulled down with protein A or protein G beads, washed and separated by SDS-PAGE. Separated proteins were transferred to membrane by standard Western blotting and proteins detected with a primary rabbit polyclonal antibody against an intracellular domain of Her2 (amino acids 975-1025; Bethyl #A300-621A) and a IRDye labeled donkey anti-rabbit secondary (LI_COR #926-32223) and visualized using a LI-COR infrared fluorescence labeling and detection system. The secondary antibody we used was 3×1:5000 IRDye 680 Donkey anti-rabbit IgG(H+L) (LI-COR #926-32223, lot B70215-O₂). The results are presented in FIG. 60.

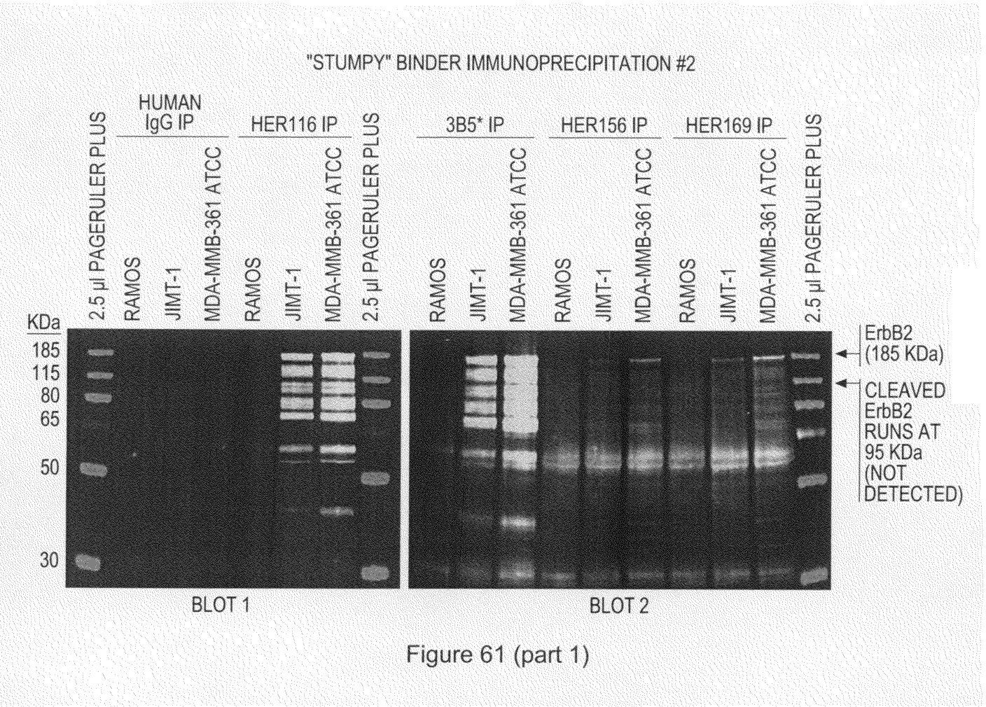

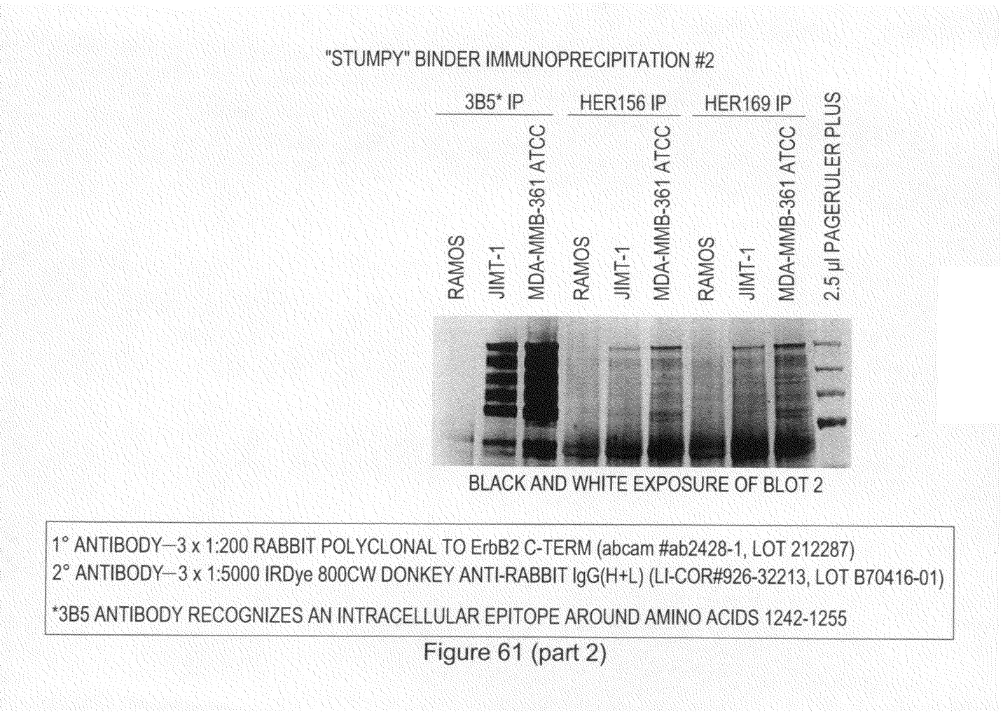

Alternatively, we solubilized Ramos, JIMT-1 and MDA-MB-361 ATCC cells with Nonidet P-40 lysis buffer (a gentler detergent than those in RIPA buffer), and immunoprecipitated HER2 from 2 mg of the lysate using 5 μg Human IgG as negative control, 5 μg SMIPs (Her116, Her156, and Her 169), and 2 μg mouse monoclonal 3B5 as positive control, respectively. We ran the immunoprecipitates on polyacrylamide gels and performed Western blotting experiment. The primary antibody we used was 3×1:200 Rabbit polyclonal to ErbB2 C-term (abcam #ab2428-1, lot 212287). The secondary antibody we used was 3×1:5000 IRDye 800CW Donkey anti-Rabbit IgG(H+L) (LI-COR #926-32213, lot B70416-01). The results are presented in FIG. 61.

We found that HER156 and HER169 are capable of binding full-length HER2. It was unclear to us whether HER156 and HER169 could bind Her2 p95 (“Stumpy;” cleaved ErbB2 that should run at 95 KDa). For example, it was not clear to us whether p95 can be immunoprecipitated at detectable levels from SKBGR3 cells by either HER156 or HER169. It was possible that there was too little p95 in SKBR3 cells for detection. We also observed that the immunoprecipitation pattern of HER116, which binds to L1/CR1 domains of Her2 not present on p95, looked the same as for 3B5, which binds intracellular domain of Her2 found on full length Her2 and p95 “stumpy.” This was possibly due to sample degradation observed post immunoprecipitation.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the application, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein.

SEQUENCE TABLE

Her2_S1R2A_CS_1F7
V_{H} with CDR1, CDR2 and CDR3 underlined
EVQLVQSGAEVKKPGASVKVSCKASGYTFTGYYMHWVRQAPGQGLEWMGWINPNSGGT
NYAQKFQGWVTMTRDTSISTAYMELSRLRSDDTAVYYCARDSTMAPGAFDIWGRGTLVTVSS (SEQ ID NO: 1)
V_{L} with CDR1, CDR2 and CDR3 underlined
QSVLTQPPSVSVAPGQTARMTCGGNNIESKTVHWYQQKPGQAPVLVVYNDNVRPSGIPAR
FSGSNSGNTATLTINRVEAGDEADYYCQVWDSSRDQGVFGGGTKLTVLGA (SEQ ID NO: 2)
Her2_S1R2A_CS_1D11
V_{H} with CDR1, CDR2 and CDR3 underlined
EVQLVQSGSEVRRPGSSVRVSCTASGDTSSSFTVNWLRQAPGQGLEWMGGITPMFGTAN
YAQMFEDRVTITADEMELSGLTSEDTAVYFCATGPSDYVWGSYRFLDTWGRGTTVTVSS (SEQ ID NO: 3)
V_{L} with CDR1, CDR2 and CDR3 underlined
QAVLTQPSSVSAAPGQEVSISCSGARSNVGGNYVSWYQHLPGTAPKLLIYDNNKRPSGMP
DRFSGSKSGTSATLGITGVQTEDEADYYCATWDSSLSAVVFGGGTKLTVLGA (SEQ ID NO: 4)
Her2_S1R2C_CS_1D3
V_{H} with CDR1, CDR2 and CDR3 underlined
QVQLVQSGSEVRRPGSSVRISCTASGDTSSSFTVNWVRQAPGQGLEWMGGITPMFGTAN
YAQVFEDRVTIIADEMELSGLTSEDTAVYFCATGPSDYVWGSYRFLDRWGRGTLVTVSS (SEQ ID NO: 5)
V_{L} with CDR1, CDR2 and CDR3 underlined
QSVLTQPPSVSAAPGQKVTISCSGGRSSIGNNYVSWYQHLPGTAPKLLIYDNNQRPSGIPD
RFSGSKSGTSATLGITGLQTGDEADYYCGTWDSSLSAVVFGGGTKVTVLGA (SEQ ID NO: 6)
Her2_S1R2C_CS_1H12
V_{H} with CDR1, CDR2 and CDR3 underlined
EVQLVETGGGLVQPGGSLRLSCAASGFTFSSYGMNWVRQAPGKGLEWVSYISSSGNTIFY
ADSVKGRFTISRDSAKNSVSLQMNSLRDEDTAVYYCASYYSYYYGMDAWGQGTMVTV (SEQ ID NO: 7)
V_{L} with CDR1, CDR2 and CDR3 underlined
SYVLTQPPSASGTPGQRVTISCSGSSSNIGSNTVNWYQQLPGTAPKLLIYSNNQRPSGVPD
RFSGSKSGTSASLAISGLRSEDEADYYCAAWDYSLSGWVFGGGTKVTVLGA (SEQ ID NO: 8)
Her2_S1R2A_CS_1D3
V_{H} with CDR1, CDR2 and CDR3 underlined
EVQLVQSGAEVKKPGASVKVSCKASGYSFTAFYIHWVRQAPGQGLEYLGWIDPNTGATKY
AQRFQGRVIMTWDTSITTATMELSRLTSDDSAVYYCVRDLREWGYELSVEYWGRGTLVTVSS (SEQ ID NO: 9)
V_{L} with CDR1, CDR2 and CDR3 underlined
QSVLTQPPSASGTPGQRVTISCSGSSSNIGSNYVYWYQQLPGTAPKLLIYRNNQRPSGVPD
RFSGSKSGTSASLAISGLRSEDEADYYCAAWDDSLSGWVFGGGTKLTVLGA (SEQ ID NO: 10)
Her2_S1R3B2_BMV_1E1
V_{H} with CDR1, CDR2 and CDR3 underlined
EVQLVETGGGVVQPGGSLSLSCAASGFTFSSYGMQWVRQAPGKGLEWVAFIRYDGSSEY
YADSVKGRFTISRDNSKNTLYLQMNSLRAEDTAVYYCGRTLESSLWGKGTLVTVSS (SEQ ID NO: 11)
V_{L} with CDR1, CDR2 and CDR3 underlined
QSVLTQPPSVSAAPGQKVTISCSGSTSNIGNNYVSWYQQHPGKAPKLMIYDVSKRPSGVP
DRFSGSKSGNSASLDISGLQSEDEADYYCAAWDDSLSEFLFGTRTKLTVLGA (SEQ ID NO: 12)
Her2_S1R3C1_CS_1D3
V_{H} with CDR1, CDR2 and CDR3 underlined
QVQLQESGPGLVKPSQTLSLTCGISGDSVSSNSAAWNWIRQSPTRGLEWLGRTYYRSSW
YHNYAPSMNSRLTIIADTSKNQFSLQLNSVTPEDTAVYYCASGWAFDVWGRGTLVTVSS (SEQ ID NO: 13)
V_{L} with CDR1, CDR2 and CDR3 underlined
QSVLTQPPSASGSPGQSVTISCTGTSSDVGAYDFVSWYQQHPGKAPKLMIYEVNKRPSGV
PDRFSGSKSGNTASLTVSGLQAEDEADYYCSSYAGSKNLLFGGGTKLTVLGA (SEQ ID NO: 14)
Her2_S1R3B2_DP47_1E8
V_{H} with CDR1, CDR2 and CDR3 underlined
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARQSGADWYFDLWGRGTLVTVSS (SEQ ID NO: 15)
V_{L} with CDR1, CDR2 and CDR3 underlined
QAVLTQPSAVSGAPGQRVTISCTGTSSNIGTNYLVHWYQQRPGTAPQLLVSGNNTRPSGV
TDRFSVSKSATSASLAITGLQAEDEADYYCQTYDINLRVWVFGGGTKVTVLGA (SEQ ID NO: 16)
Her2_S1R3B2_BMV_1G2
V_{H} with CDR1, CDR2 and CDR3 underlined
QVQLVQSGAEVKKPGSSVKVSCKASGYTFTSYGISWVRQAPGQGLEWMGWISAYNGNTN
YAQKLQGRVTMTTDTSTSTAYMELRSLRSDDTAVYYCARVPGVSGSYPDYYYMDVWGKGTLVTVSS (SEQ ID NO: 17)
V_{L} with CDR1, CDR2 and CDR3 underlined
DIQMTQSPSTLSASIGDRVTITCRASEGIYHWLAWYQQKPGKAPKLLIYKASSLASGAPSRF
SGSGSGTDFTLTISSLQPDDFATYYCQQYSNYPLTFGGGTKLEIKRA (SEQ ID NO: 18)
Her2_S1R3B2_BMV_1H5
V_{H} with CDR1, CDR2 and CDR3 underlined
EVQLVQSGGGLVRPGGSLRLSCAASGFSFSDYYMTWIRQIPGKGLEWVAVIWNDGSDRYY
ADSVKGRFTISRDNSKNTLFLQMSSLRDEDTALYYCVRGGPTASSGFDYWGRGTLVTVSS (SEQ ID NO: 19)
V_{L} with CDR1, CDR2 and CDR3 underlined
SSELTQPASVSGSPGQSITISCTGTSSDVGGYNYVSWYLQHPGKAPKLMIYEGSKRPSGVS
NRFSGSKSGNTASLTISGLQAEDEADYYCSSYTTRSTRVFGGGTKLTVLGA (SEQ ID NO: 20)
Her2_S1R3C1_CS_1A6
V_{H} with CDR1, CDR2 and CDR3 underlined
EVQLVQSGAEVKKPGESLKISCKGFGYNFRSAWIGWVRQMPGKGLEWMGVIYPGDSDVR
YSPSFQGQVTISADKSISTAYLQWSSLKASDTAMYYCTRPVGQWVDSDYWGKGTLVTVSS (SEQ ID NO: 21)
V_{L} with CDR1, CDR2 and CDR3 underlined
QSVLTQPPSASGTPGQRVTISCSGSSSNIGTNTVNWYQQLPGTAPKLLIYTSNQRPSGVPA
RFSASNSGTSASLAISGLRSEDEADYYCAAWDDKLSGAVFGGGTKLTVLGA (SEQ ID NO: 22)
Her2_S1R3B2_DP47_1C9
V_{H} with CDR1, CDR2 and CDR3 underlined
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARWRPLLDYHFDQWGQGTMVTVSS (SEQ ID NO: 23)
V_{L} with CDR1, CDR2 and CDR3 underlined
QSVLTQPPSASGTPGQTVTISCSGSSSNIGSSVVNWYQQF0PGTAPKVLVYSNTQRPSGVP
DRFSGSRSGTSASLAISGLQSEDEADYYCLAWDASLNGWVFGGGTKLTVLGA (SEQ ID NO: 24)
Her2_S1R3B2_DP47_1E10
V_{H} with CDR1, CDR2 and CDR3 underlined
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARGYSGYDDPDSWGRGTTVTVSS (SEQ ID NO: 25)
V_{L} with CDR1, CDR2 and CDR3 underlined
HVILTQPPSTSGTPGQTVTISCSGSSSNIGSHYVYWYQQLPGTAPKLLIYRNNQRPSGVPD
RFSGSKSGTSASLAISGLRSEDETDYYCAAWDDSLSGRVFGTGTKLTVLGA (SEQ ID NO: 26)
Her2_S1R3C1_CS_1B10
V_{H} with CDR1, CDR2 and CDR3 underlined
QVQLQQSGAEVKKPGSSVKVSCKASGGTISNYAISWVRLAPGQGLEWMGSIVPLHGTTNF
AQKFQGRVTITADESTSTSYMEVNVLTYEDTAMYYCASLNWGYWGRGTLVTVSS (SEQ ID NO: 27)
V_{L} with CDR1, CDR2 and CDR3 underlined
NFMLTQPHSVSESPGKTVTISCTGSSGSIASNYVQWYQQRPDSAPTTVIYEDNRRSSGVPD
RFSGSIDSNSASLSISGLKTEDEADYYCQSYDSSGHVVFGGGTKLTVLGA (SEQ ID NO: 28)
Her2_S1R3A1_BMV_1F3
V_{H} with CDR1, CDR2 and CDR3 underlined
EVQLVESGEGLVKPGGSLRLSCTASGFTFRSYSLNWVRQAPGQGLEWVSSISSTSTYIYYA
DSVKGRFTISRDDAKNTLYLQMNSLRAEDTAAYYCVRLGSGGGYFPDYWGRGTLVTVSS (SEQ ID NO: 29)
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCNSRDSSGNHVVFGGGTKLTVLGA (SEQ ID NO: 30)
Her2_S1R3B1_BMV_1G11
V_{H} with CDR1, CDR2 and CDR3 underlined
QVQLVQSGGGLVQPGGSLRLSCAASGFTFSTYAMSWARQAPGKGLEWVSSISGDGGRIL
DADSAKGRFTISRDNSKNTLYLQMNGLRVEDTALYYCARADGNYWGRGTMVTVSS (SEQ ID NO: 31)
V_{L} with CDR1, CDR2 and CDR3 underlined
QSVLTQPASVSGSPGQSITISCTGTSSDVGGYNYVSWYQQHPGKAPKLMIYEGSKRPSGV
SNRFSGSKSGNTASLTISGLQAEDEADYYCSSYTTRSTRVFGGGTKLTVLGA (SEQ ID NO: 32)
Her2_S1R3A1_BMV_1G4
V_{H} with CDR1, CDR2 and CDR3 underlined
QVQLVESGAEVKKPGASVKVSCKASGYTFTSYDINWVRQAPGQRLEWMGWINAGNGNTK
YSQKFQGRVTITRDTSASTAYMELRSLRSDDTAVYYCARGRSYGHPYYFDYWGQGTLVTVSS (SEQ ID NO: 33)
V_{L} with CDR1, CDR2 and CDR3 underlined
QSVLTQPASVSGSPGQSITISCTGTSSDVGGYNYVSWYQQHPGKAPKLMIYEGSKRPSGV
SNRFSGSKSGNTASLTISGLQAEDEADYYCSSYTTRSTRVFGGGTKLTVLGA (SEQ ID NO: 34)
Her2_S1R3B1_BMV_1H11
V_{H} with CDR1, CDR2 and CDR3 underlined
EVQLVQSGGGLVKPGGSLRLSCAASGFTFSSYGMHWVRQAPGKGLEWVAGIFYDGGNKY
YADSVKGRFTISRDNSKNTLYLQMNSLRAEDTAVYYCARDRGYYYMDVWGKGTTVTVSS (SEQ ID NO: 35)
V_{L} with CDR1, CDR2 and CDR3 underlined
QSVLTQPPSVSGAPGQRVTISCTGRSSNIGAGHDVHWYQQLPGTAPKLLIYGDSNRPSGV
PDRFSGSRSGTSASLAITGLQAEDEADYYCQSYDSSLRGSVFGGGTKVTVLGA (SEQ ID NO: 36)
Her2_S1R3A1_CS_1B9
V_{H} with CDR1, CDR2 and CDR3 underlined
KVQLVQSGTEVKKPGESLKISCQGSGYRFSSDWIAWVRQMPGKGLEWMGIVYPGDSDTR
YSPSFQGQVTISADKSISTAYLQWSGLKASDTAKYYCARVQQAVGAKGYAMDVWGKGTLVTVSS (SEQ ID NO: 37)
V_{L} with CDR1, CDR2 and CDR3 underlined
QTVVIQEPSFSVSPGGTVTLTCGLSSGSVSTSYYPSWYRQTPGQAPHTLIHNTKIRSSGVP
DRFSGSILGNNAALTITGAQADDESDYYCLLYMGSGIYVFGGGTKLTVLGA (SEQ ID NO: 38)
Her2_S1R3B1_BMV_1H9
V_{H} with CDR1, CDR2 and CDR3 underlined
QVQLQESGAGLVKPSGTLSLTCAVSGGSISSGNWWSWVRQPPGKGLEWIGEISHSGSTN
YNPSLKSRVTISVDKSKNQFSLNLSSVTAADTAVYYCARVRGTVGDTRGPDYWGQGTLVTVSS (SEQ ID NO: 39)
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCNSRDSSGNHVVFGGGTKLTVLGA (SEQ ID NO: 40)
Her2_S1R3A1_CS_1B10
V_{H} with CDR1, CDR2 and CDR3 underlined
EVQLVQSGAEVKKPGASVRVSCKGSGNTFTGHYIHWVRQAPGQGLEWLGWIDPNTGDIQ
YSENFKGSVTLTRDPSINSVFMDLIRLTSDDTAMYYCAREGAGLANYYYYGLDVWGRGTMVTVSS (SEQ ID NO: 41)
V_{L} with CDR1, CDR2 and CDR3 underlined
QTVVLQEPSFSVSPGGTVTLTCGLNFGSVSTAYYPSWYQQTPGQAPRTLIYGTNIRSSGVP
DRFSGSIVGNKAALTITGAQTEDESDYYCALYMGSGMLFGGGTKVTVLGA (SEQ ID NO: 42)
Her2_S1R3B1_BMV_1C12
V_{H} with CDR1, CDR2 and CDR3 underlined
EVQLVQSGGGVVQPGRSLRLSCAASGFTFSSYGMHWVRQAPGKGLEWVAVISYDGSIKY
YADSVKGRFTISRDNSKNTLYLQMNSLRAEDTAVYYCARTGEYSGYDTSGYSNWGQGTLVTVSS (SEQ ID NO: 43)
V_{L} with CDR1, CDR2 and CDR3 underlined
QSVLTQPPSASGTPGQRVTISCSGSSSNIGSNTVNWYQRLPGAAPQLLIYNNDQRPSGIPD
RFSGSKSGTSGSLVISGLQSEDEADYYCASWDDSLNGRVFGGGTKLTVLG (SEQ ID NO: 44)
Her2_S1R3C1_BMV_1H11
V_{H} with CDR1, CDR2 and CDR3 underlined
GVQLVESGGGLVKPGGSLRLSCAASGFTFSSYNMNWVRQAPGKGLEWVSAISGSGGSTY
YADSVTGRFTISRDNSKNTLYLQMNSLRAEDTAVYYCAKDTSGWYGDGMDVWGRGTLVTVSS (SEQ ID NO: 45)
V_{L} with CDR1, CDR2 and CDR3 underlined
DIQMTQSPSTLSASIGDRVTITCRASEGIYHWLAWYQQKPGKAPKLLIYKASSLASGAPSRF
SGSGSGTDFTLTISSLQPDDFATYYCQQYSNYPLTFGGGTKLEIKRA (SEQ ID NO: 46)
Her2_S1R3B1_BMV_1A10
V_{H} with CDR1, CDR2 and CDR3 underlined
QMQLVQSGGGVVQPGRSLRLSCAASGFTFSSYGMHWVRQAPGKGLEWVAVISYDGSIKY
YADSVKGRFTISRDNSKNTLYLQMNSLRAEDTGVYYCSKDRYSSGWYSSDAFDIWGRGTMVTVSS (SEQ ID NO: 47)
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCHSRDSSGNHVLFGGGTKLTVLGA (SEQ ID NO: 48)
Her2_S1R3A1_CS_1D11
V_{H} with CDR1, CDR2 and CDR3 underlined
EVQLVQSGAEVKKPGESLKISCKGSGYTFTNHWIAWVRQMPGKGLEWMGIIYPGDSETRY
SPSFQGHVTISADKSISTAYLQWSTLKDSDSAMYFCVRQARGWDDGRAGYYYSGMDAWGQGTLVTVSS (SEQ ID NO: 49)
V_{L} with CDR1, CDR2 and CDR3 underlined
QAVVLQEPSFSVSPGGTVTLTCGLRSGSVSTSHYPSWYQQTPGQAPRTLIYSTNTRSSGV
PDRFSGSILGNKAALTITGAQADDESNYYCMLYMGSGMYVFGGGTKVTVLGA (SEQ ID NO: 50)
Her2_S1R3C1_DP47_1H1
V_{H} with CDR1, CDR2 and CDR3 underlined
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARVSGSHFPFFDSWGQGTMVTVSS (SEQ ID NO: 51)
V_{L} with CDR1, CDR2 and CDR3 underlined
QSVLTQPPSVSVAPGQTARITCGGDKIGHKSVHWYQQKPGQAPVLLVYDDRKRPSGIPER
FSGSNSGNTATLTISRVEAGDEAAYHCQVWDRSSDPYVFGTGTKVTVLGA (SEQ ID NO: 52)
Her2_S1R3A1_CS_1B12
V_{H} with CDR1, CDR2 and CDR3 underlined
QVQLVQSGAEVKKPGASVKVSCQASGYTFSGHYMHLVRQAPGQGLEWMGWIHPTSGGT
TYAQKFQGRVVMTRDTSISTAYMELSRLTSDDTAVYYCARMSQNYDAFDIWGQGTMVTVSS (SEQ ID NO: 53)
V_{L} with CDR1, CDR2 and CDR3 underlined
QAVLTQPSSVSGAPGQRVTISCTGSSSNIGAGYDVNWYQQFPGTAPKIIVYGDRPSGAPDR
FSGSKSGTSASLAITGLRAEDEADYYCQSWDSRLSSYVFGTGTKVTVLGA (SEQ ID NO: 54)
Her2_S1R3B1_BMV_1H5
V_{H} with CDR1, CDR2 and CDR3 underlined
QVQLQESGGGVVQPGGSLRLSCAASGFTFSGYGMHWVRQAPGKGLEWVASVRNDGSNT
YYTDSVKDRFTISRDNTKNTLYLQMNSLRAEDTAVYYCAKSRRVMYGTSYYFDYWGRGTLVTVSS (SEQ ID NO: 55)
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCNSRDSSGNHVVFGGGTKLTVLGA (SEQ ID NO: 56)
Her2_S1R3A1_DP47_1A6
V_{H} with CDR1, CDR2 and CDR3 underlined
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARDLGIDPLWSGYYTPLDYWGRGTMVTVSS (SEQ ID NO: 57)
V_{L} with CDR1, CDR2 and CDR3 underlined
HVILTQPPSASGTPGQRVTISCSGSSSNIGSNSVSWYQQLPGTAPKLLMYTNNQRPSGVPD
RFSGSKSGTSASLAISGLQSEDEADYYCATWDASLNTWVFGGGTKVTVLGA (SEQ ID NO: 58)
Her2_S1R3B1_DP47_1E1
V_{H} with CDR1, CDR2 and CDR3 underlined
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARGGSGSDYWGQGTMVTVSS (SEQ ID NO: 59)
V_{L} with CDR1, CDR2 and CDR3 underlined
NFMLTQPHSVSGSPGKTVTISCTRSSGYIDSKYVQWYQQRPGSAPTTVIYEDNRRPSGVP
DRFSGSIDSNSASLTISGLETEDEADYYCQSYDDTNVVFGGGTKVTVLGA (SEQ ID NO: 60)
Her2_S1R3B1_BMV_1A1
V_{H} with CDR1, CDR2 and CDR3 underlined
EVQLVQSGAEVKEPGASVKVSCKASGYDFSNYGFSWVRQAPGQGLEWMGWISSYNGYT
NYAQRLQGRVTMTTDTSTSTAYMELRSLRSDDTAVYYCARDRGLGNWYFDLWGQGTLVTVSS (SEQ ID NO: 61)
V_{L} with CDR1, CDR2 and CDR3 underlined
QSVLTQPASVSGSPGQSITISCTGTSSDVGGYNYVSWYQQHPGKAPKLMIYEGSKRPSGV
SNRFSGSKSGNTASLTISGLQAEDEADYYCSSYTTRSTRVFGGGTKLTVLGA (SEQ ID NO: 62)
Her2_S1R2A_CS_1F7
V_{L} with CDR1, CDR2 and CDR3 underlined
QSVLTQPPSVSVAPGQTARMTCGGNNIESKTVHWYQQKPGQAPVLVVYNDNVRPSGIPAR
FSGSNSGNTATLTINRVEAGDEADYYCQVWDSSRDQGVFGGGTKLTVL (SEQ ID NO: 63)
Her2_S1R2A_CS_1D11
V_{L} with CDR1, CDR2 and CDR3 underlined
QAVLTQPSSVSAAPGQEVSISCSGARSNVGGNYVSWYQHLPGTAPKLLIYDNNKRPSGMP
DRFSGSKSGTSATLGITGVQTEDEADYYCATWDSSLSAVVFGGGTKLTVL (SEQ ID NO: 64)
Her2_S1R2C_CS_1D3
V_{H} with CDR1, CDR2 and CDR3 underlined
QVQLVQSGSEVRRPGSSVRISCTASGDTSSSFTVNWVRQAPGQGLEWMGGITPMFGTAN
YAQVFEDRVTIIADEMELSGLTSEDTAVYFCATGPSDYVWGSYRFLDNWGRGTLVTVSS (SEQ ID NO: 65)
Her2_S1R2C_CS_1D3
V_{L} with CDR1, CDR2 and CDR3 underlined
QSVLTQPPSVSAAPGQKVTISCSGGRSSIGNNYVSWYQHLPGTAPKLLIYDNNQRPSGIPD
RFSGSKSGTSATLGITGLQTGDEADYYCGTWDSSLSAVVFGGGTKVTVL (SEQ ID NO: 66)
Her2_S1R2C_CS_1H12
V_{H} with CDR1, CDR2 and CDR3 underlined
EVQLVETGGGLVQPGGSLRLSCAASGFTFSSYGMNWVRQAPGKGLEWVSYISSSGNTIFY
ADSVKGRFTISRDSAKNSVSLQMNSLRDEDTAVYYCASYYSYYYGMDAWGQGTMVTVSS (SEQ ID NO: 67)
Her2_S1R2C_CS_1H12
V_{L} with CDR1, CDR2 and CDR3 underlined
SYVLTQPPSASGTPGQRVTISCSGSSSNIGSNTVNWYQQLPGTAPKLLIYSNNQRPSGVPD
RFSGSKSGTSASLAISGLRSEDEADYYCAAWDYSLSGWVFGGGTKVTVL (SEQ ID NO: 68)
Her2_S1R2A_CS_1D3
V_{L} with CDR1, CDR2 and CDR3 underlined
QSVLTQPPSASGTPGQRVTISCSGSSSNIGSNYVYWYQQLPGTAPKLLIYRNNQRPSGVPD
RFSGSKSGTSASLAISGLRSEDEADYYCAAWDDSLSGWVFGGGTKLTVL (SEQ ID NO: 69)
Her2_S1R3B2_BMV_1E1
V_{L} with CDR1, CDR2 and CDR3 underlined
QSVLTQPPSVSAAPGQKVTISCSGSTSNIGNNYVSWYQQHPGKAPKLMIYDVSKRPSGVP
DRFSGSKSGNSASLDISGLQSEDEADYYCAAWDDSLSEFLFGTRTKLTVL (SEQ ID NO: 70)
Her2_S1R3C1_CS_1D3
V_{L} with CDR1, CDR2 and CDR3 underlined
QSVLTQPPSASGSPGQSVTISCTGTSSDVGAYDFVSWYQQHPGKAPKLMIYEVNKRPSGV
PDRFSGSKSGNTASLTVSGLQAEDEADYYCSSYAGSKNLLFGGGTKLTVL (SEQ ID NO: 71)
Her2_S1R3B2_DP47_1E8
V_{L} with CDR1, CDR2 and CDR3 underlined
QAVLTQPSAVSGAPGQRVTISCTGTSSNIGTNYLVHWYQQRPGTAPQLLVSGNNTRPSGV
TDRFSVSKSATSASLAITGLQAEDEADYYCQTYDINLRVWVFGGGTKVTVL (SEQ ID NO: 72)
Her2_S1R3B2_BMV_1G2
V_{L} with CDR1, CDR2 and CDR3 underlined
DIQMTQSPSTLSASIGDRVTITCRASEGIYHWLAWYQQKPGKAPKLLIYKASSLASGAPSRF
SGSGSGTDFTLTISSLQPDDFATYYCQQYSNYPLTFGGGTKLEIK (SEQ ID NO: 73)
Her2_S1R3B2_BMV_1H5
V_{L} with CDR1, CDR2 and CDR3 underlined
SSELTQPASVSGSPGQSITISCTGTSSDVGGYNYVSWYLQHPGKAPKLMIYEGSKRPSGVS
NRFSGSKSGNTASLTISGLQAEDEADYYCSSYTTRSTRVFGGGTKLTVL (SEQ ID NO: 74)
Her2_S1R3C1_CS_1A6
V_{L} with CDR1, CDR2 and CDR3 underlined
QSVLTQPPSASGTPGQRVTISCSGSSSNIGTNTVNWYQQLPGTAPKLLIYTSNQRPSGVPA
RFSASNSGTSASLAISGLRSEDEADYYCAAWDDKLSGAVFGGGTKLTVL (SEQ ID NO: 75)
Her2_S1R3B2_DP47_1C9
V_{L} with CDR1, CDR2 and CDR3 underlined
QSVLTQPPSASGTPGQTVTISCSGSSSNIGSSVVNWYQQFPGTAPKVLVYSNTQRPSGVP
DRFSGSRSGTSASLAISGLQSEDEADYYCLAWDASLNGWVFGGGTKLTVL (SEQ ID NO: 76)
Her2_S1R3B2_DP47_1E10
V_{L} with CDR1, CDR2 and CDR3 underlined
HVILTQPPSTSGTPGQTVTISCSGSSSNIGSHYVYWYQQLPGTAPKLLIYRNNQRPSGVPD
RFSGSKSGTSASLAISGLRSEDETDYYCAAWDDSLSGRVFGTGTKLTVL (SEQ ID NO: 77)
Her2_S1R3C1_CS_1B10
V_{L} with CDR1, CDR2 and CDR3 underlined
NFMLTQPHSVSESPGKTVTISCTGSSGSIASNYVQWYQQRPDSAPTTVIYEDNRRSSGVPD
RFSGSIDSNSASLSISGLKTEDEADYYCQSYDSSGHVVFGGGTKLTVL (SEQ ID NO: 78)
Her2_S1R3A1_BMV_1F3
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCNSRDSSGNHVVFGGGTKLTVL (SEQ ID NO: 79)
Her2_S1R3B1_BMV_1G11
V_{L} with CDR1, CDR2 and CDR3 underlined
QSVLTQPASVSGSPGQSITISCTGTSSDVGGYNYVSWYQQHPGKAPKLMIYEGSKRPSGV
SNRFSGSKSGNTASLTISGLQAEDEADYYCSSYTTRSTRVFGGGTKLTVL (SEQ ID NO: 80)
Her2_S1R3A1_BMV_1G4
V_{L} with CDR1, CDR2 and CDR3 underlined
QSVLTQPASVSGSPGQSITISCTGTSSDVGGYNYVSWYQQHPGKAPKLMIYEGSKRPSGV
SNRFSGSKSGNTASLTISGLQAEDEADYYCSSYTTRSTRVFGGGTKLTVL (SEQ ID NO: 81)
Her2_S1R3B1_BMV_1H11
V_{L} with CDR1, CDR2 and CDR3 underlined
QSVLTQPPSVSGAPGQRVTISCTGRSSNIGAGHDVHWYQQLPGTAPKLLIYGDSNRPSGV
PDRFSGSRSGTSASLAITGLQAEDEADYYCQSYDSSLRGSVFGGGTKVTVL (SEQ ID NO: 82)
Her2_S1R3A1_CS_1B9
V_{L} with CDR1, CDR2 and CDR3 underlined
QTVVIQEPSFSVSPGGTVTLTCGLSSGSVSTSYYPSWYRQTPGQAPHTLIHNTKIRSSGVP
DRFSGSILGNNAALTITGAQADDESDYYCLLYMGSGIYVFGGGTKLTVL (SEQ ID NO: 83)
Her2_S1R3B1_BMV_1H9
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCNSRDSSGNHVVFGGGTKLTVL (SEQ ID NO: 84)
Her2_S1R3A1_CS_1B10
V_{L} with CDR1, CDR2 and CDR3 underlined
QTVVLQEPSFSVSPGGTVTLTCGLNFGSVSTAYYPSWYQQTPGQAPRTLIYGTNIRSSGVP
DRFSGSIVGNKAALTITGAQTEDESDYYCALYMGSGMLFGGGTKVTVL (SEQ ID NO: 85)
Her2_S1R3B1_BMV_1C12
V_{L} with CDR1, CDR2 and CDR3 underlined
QSVLTQPPSASGTPGQRVTISCSGSSSNIGSNTVNWYQRLPGAAPQLLIYNNDQRPSGIPD
RFSGSKSGTSGSLVISGLQSEDEADYYCASWDDSLNGRVFGGGTKLTVL (SEQ ID NO: 86)
Her2_S1R3C1_BMV_1H11
V_{L} with CDR1, CDR2 and CDR3 underlined
DIQMTQSPSTLSASIGDRVTITCRASEGIYHWLAWYQQKPGKAPKLLIYKASSLASGAPSRF
SGSGSGTDFTLTISSLQPDDFATYYCQQYSNYPLTFGGGTKLEIK (SEQ ID NO: 87
Her2_S1R3B1_BMV_1A10
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCHSRDSSGNHVLFGGGTKLTVL (SEQ ID NO: 88)
Her2_S1R3A1_CS_1D11
V_{L} with CDR1, CDR2 and CDR3 underlined
QAVVLQEPSFSVSPGGTVTLTCGLRSGSVSTSHYPSWYQQTPGQAPRTLIYSTNTRSSGV
PDRFSGSILGNKAALTITGAQADDESNYYCMLYMGSGMYVFGGGTKVTVL (SEQ ID NO: 89)
Her2_S1R3C1_DP47_1H1
V_{L} with CDR1, CDR2 and CDR3 underlined
QSVLTQPPSVSVAPGQTARITCGGDKIGHKSVHWYQQKPGQAPVLLVYDDRKRPSGIPER
FSGSNSGNTATLTISRVEAGDEAAYHCQVWDRSSDPYVFGTGTKVTVL (SEQ ID NO: 90)
Her2_S1R3A1_CS_1B12
V_{L} with CDR1, CDR2 and CDR3 underlined
QAVLTQPSSVSGAPGQRVTISCTGSSSNIGAGYDVNWYQQFPGTAPKIIVYGDRPSGAPDR
FSGSKSGTSASLAITGLRAEDEADYYCQSWDSRLSSYVFGTGTKVTVL (SEQ ID NO: 91)
Her2_S1R3B1_BMV_1H5
V_{L} with CDR1, CDR2 and CDR3 underlined
SSELTQDPAVSVALGQTVRITCQGDSLRSYYASWYQQKPGQAPVLVIYGKNNRPSGIPDRF
SGSSSGNTASLTITGAQAEDEADYYCNSRDSSGNHVVFGGGTKLTVL (SEQ ID NO: 92)
Her2_S1R3A1_DP47_1A6
V_{L} with CDR1, CDR2 and CDR3 underlined
HVILTQPPSASGTPGQRVTISCSGSSSNIGSNSVSWYQQLPGTAPKLLMYTNNQRPSGVPD
RFSGSKSGTSASLAISGLQSEDEADYYCATWDASLNTWVFGGGTKVTVL (SEQ ID NO: 93)
Her2_S1R3B1_DP47_1E1
V_{L} with CDR1, CDR2 and CDR3 underlined
NFMLTQPHSVSGSPGKTVTISCTRSSGYIDSKYVQWYQQRPGSAPTTVIYEDNRRPSGVP
DRFSGSIDSNSASLTISGLETEDEADYYCQSYDDTNVVFGGGTKVTVL (SEQ ID NO: 94)
Her2_S1R3B1_BMV_1A1
V_{L} with CDR1, CDR2 and CDR3 underlined
QSVLTQPASVSGSPGQSITISCTGTSSDVGGYNYVSWYQQHPGKAPKLMIYEGSKRPSGV
SNRFSGSKSGNTASLTISGLQAEDEADYYCSSYTTRSTRVFGGGTKLTVL (SEQ ID NO 95)
Her2_S1R2A_CS_1F7
V_{H} with CDR1, CDR2 and CDR3 underlined
GAGGTCCAGCTGGTGCAGTCTGGGGCTGAGGTGAAGAAGCCTGGGGCCTCAGTGAAG
GTCTCCTGCAAGGCTTCTGGATACACCTTCACCGGCTACTATATGCACTGGGTGCGAC
AGGCCCCTGGACAAGGGCTTGAGTGGATGGGATGGATCAACCCTAACAGTGGTGGCA
CAAACTATGCACAGAAGTTTCAGGGCTGGGTCACCATGACCAGGGACACGTCCATCAG
CACAGCCTACATGGAGCTGAGCAGGCTGAGATCTGACGACACGGCCGTGTATTACTGT
GCGAGAGATTCTACTATGGCCCCAGGTGCTTTTGATATCTGGGGCCGAGGCACCCTGG
TCACCGTCTCGAGT (SEQ ID NO: 96)
Her2_S1R2A_CS_1F7
V_{L} with CDR1, CDR2 and CDR3 underlined
CAGTCTGTGCTGACTCAGCCACCCTCGGTGTCAGTGGCCCCAGGACAGACGGCCAGG
ATGACCTGTGGGGGAAACAACATTGAAAGTAAAACTGTGCATTGGTACCAGCAGAAGC
CGGGCCAGGCCCCTGTGCTGGTCGTCTACAATGATAACGTCCGGCCCTCAGGGATCC
CTGCGCGATTCTCTGGCTCCAACTCCGGCAACACGGCCACCCTGACCATCAACAGGGT
CGAAGCCGGGGATGAGGCCGACTATTATTGTCAGGTGTGGGACTCCAGTAGAGATCAA
GGGGTATTCGGCGGAGGGACCAAGCTGACCGTC (SEQ ID NO: 97)
Her2_S1R2A_CS_1D11
V_{H} with CDR1, CDR2 and CDR3 underlined
GGAGGCCTGGGTCCTCGGTGAGGGTCTCCTGCACGGCTTCTGGAGACACCTCCAGCA
GCTTTACCGTCAACTGGCTGCGACAGGCCCCTGGACAAGGTCTTGAGTGGATGGGAG
GGATCACCCCTATGTTTGGCACTGCAAACTACGCACAGATGTTCGAGGACAGAGTCAC
GATAACCGCGGACGAAATGGAACTGAGTGGCCTGACATCTGAGGACACGGCCGTGTAT
TTTTGTGCGACAGGCCCCTCCGATTACGTTTGGGGGAGTTATCGTTTCCTTGACACCTG
GGGGCGGGGGACCACGGTCACCGTCTCGAGT (SEQ ID NO: 98)
Her2_S1R2A_CS_1D11
V_{L} with CDR1, CDR2 and CDR3 underlined
CAGGCTGTGCTGACTCAGCCGTCCTCAGTGTCTGCGGCCCCAGGACAGGAGGTCTCC
ATCTCCTGCTCTGGAGCCAGATCCAACGTTGGGGGTAATTATGTTTCCTGGTACCAACA
CCTCCCAGGAACAGCCCCCAAACTCCTCATTTATGACAATAATAAGCGACCCTCAGGGA
TGCCTGACCGATTCTCTGGCTCCAAGTCTGGCACGTCAGCCACCCTGGGCATCACCGG
AGTCCAGACTGAGGACGAGGCCGATTATTACTGCGCAACATGGGATAGCAGCCTGAGC
GCTGTGGTCTTCGGCGGAGGGACCAAGCTGACCGTCCTA (SEQ ID NO: 99)
Her2_S1R2C_CS_1D3
V_{H} with CDR1, CDR2 and CDR3 underlined
CAGGTGCAGCTGGTGCAGTCTGGGTCTGAGGTGAGGAGGCCTGGGTCCTCGGTGAGG
ATCTCCTGCACGGCTTCTGGAGACACCTCCAGCAGCTTTACCGTCAACTGGGTGCGAC
AGGCCCCTGGACAAGGTCTTGAGTGGATGGGAGGGATCACCCCTATGTTTGGCACTGC
AAACTACGCACAGGTGTTCGAGGACAGAGTCACAATAATCGCGGACGAGATGGAACTG
AGTGGCCTGACATCTGAGGACACGGCCGTGTATTTCTGTGCGACAGGCCCCTCCGATT
ACGTTTGGGGGAGTTATCGTTTCCTTGACAACTGGGGCAGGGGCACCCTGGTCACCGT
CTCGAGT (SEQ ID NO: 100)
Her2_S1R2C_CS_1D3
V_{L} with CDR1, CDR2 and CDR3 underlined
CAGTCTGTGCTGACTCAGCCACCCTCAGTGTCTGCGGCCCCAGGGCAGAAGGTCACC
ATCTCCTGCTCTGGAGGCAGGTCCAGCATTGGGAATAATTATGTGTCCTGGTATCAACA
CCTCCCAGGAACAGCCCCCAAACTCCTCATCTATGACAATAATCAGCGACCCTCAGGG
ATTCCTGACCGATTCTCTGGCTCCAAGTCTGGCACGTCAGCCACCCTGGGCATCACCG
GACTCCAGACTGGGGACGAGGCCGATTATTACTGCGGAACATGGGATAGCAGCCTGA
GTGCTGTGGTGTTTGGCGGAGGGACCAAGGTCACCGTCCTA (SEQ ID NO: 101)
Her2_S1R2C_CS_1H12
V_{H} with CDR1, CDR2 and CDR3 underlined
GAGGTGCAGCTGGTGGAGACTGGGGGAGGCTTGGTACAGCCTGGGGGGTCCCTGAG
ACTCTCCTGTGCAGCCTCTGGATTCACCTTCAGTAGCTATGGCATGAACTGGGTCCGC
CAGGCTCCAGGGAAGGGGCTGGAGTGGGTTTCATACATTAGTAGTTCTGGTAATACCA
TATTCTACGCAGACTCTGTGAAGGGCCGATTCACCATCTCCAGAGACAGTGCCAAGAAT
TCAGTGTCTCTGCAGATGAACAGCCTGAGAGACGAGGACACGGCTGTGTATTACTGTG
CTTCCTACTACTCCTACTACTACGGTATGGACGCCTGGGGCCAGGGGACAATGGTCAC
CGTCTCGAGTTCGAGT (SEQ ID NO: 102)
Her2_S1R2C_CS_1H12
V_{L} with CDR1, CDR2 and CDR3 underlined
TCTGGGACCCCCGGGCAGAGGGTCACCATCTCTTGTTCTGGAAGCAGCTCCAACATCG
GAAGTAATACTGTAAACTGGTACCAGCAGCTCCCAGGAACGGCCCCCAAACTCCTCAT
CTATAGTAATAATCAGCGGCCCTCAGGGGTCCCTGACCGATTCTCTGGCTCCAAGTCT
GGCACCTCAGCCTCCCTGGCCATCAGTGGGCTGCGGTCCGAGGATGAGGCTGATTATT
ACTGTGCAGCATGGGATTACAGCCTGAGTGGTTGGGTGTTCGGCGGAGGGACCAAGG
TCACCGTCCTA (SEQ ID NO: 103)
Her2_S1R2A_CS_1D3
V_{H} with CDR1, CDR2 and CDR3 underlined
GAAGTGCAGCTGGTGCAGTCTGGGGCTGAGGTGAAGAAGCCTGGGGCCTCAGTGAAG
GTCTCCTGCAAGGCTTCTGGGTACAGCTTCACCGCCTTCTATATTCACTGGGTGCGACA
GGCCCCTGGACAAGGCCTTGAGTATTTGGGATGGATCGACCCTAATACTGGTGCCACA
AAATATGCACAGCGCTTTCAGGGCAGGGTCATCATGACCTGGGACACGTCCATCACCA
CAGCCACCATGGAACTGAGCAGGCTGACGTCTGACGACTCGGCCGTCTACTACTGTGT
GAGAGATTTGCGGGAGTGGGGCTACGAATTGTCCGTTGAGTATTGGGGCAGAGGAAC
CCTGGTCACCGTCTCGAGT (SEQ ID NO: 104)
Her2_S1R2A_CS_1D3
V_{L} with CDR1, CDR2 and CDR3 underlined
CAGTCTGTGCTGACTCAGCCACCCTCAGCGTCTGGGACCCCCGGGCAGAGGGTCACC
ATCTCTTGTTCTGGAAGCAGCTCCAACATCGGAAGTAATTATGTATACTGGTACCAGCA
GCTCCCAGGAACGGCCCCCAAACTCCTCATCTATAGGAATAATCAGCGGCCCTCAGGG
GTCCCTGACCGATTCTCTGGCTCCAAGTCTGGCACCTCAGCCTCCCTGGCCATCAGTG
GGCTCCGGTCCGAGGATGAGGCTGATTATTACTGTGCAGCATGGGATGACAGCCTGAG
TGGTTGGGTGTTCGGCGGAGGGACCAAGCTGACCGTCCTA (SEQ ID NO: 105)
Her2_S1R3B2_BMV_1E1
V_{H} with CDR1, CDR2 and CDR3 underlined
GAGGTGCAGCTGGTGGAGACTGGGGGAGGCGTGGTCCAGCCTGGGGGGTCCCTGAG
CCTCTCCTGTGCAGCGTCTGGATTCACCTTCAGTAGCTATGGCATGCAGTGGGTCCGC
CAGGCTCCAGGCAAGGGGCTGGAGTGGGTGGCGTTTATACGGTACGATGGAAGTAGT
GAATACTATGCAGACTCCGTGAAGGGCCGATTCACCATCTCCAGAGACAATTCCAAGAA
CACGCTGTATCTGCAAATGAACAGCCTGAGAGCTGAGGACACGGCTGTGTATTACTGT
GGAAGAACGCTGGAGTCTAGTTTGTGGGGCAAGGGAACCCTGGTCACCGTCTCGAGT (SEQ ID NO: 106)
Her2_S1R3B2_BMV_1E1
V_{L} with CDR1, CDR2 and CDR3 underlined
CAGTCTGTGTTGACGCAGCCGCCCTCAGTGTCTGCGGCCCCAGGACAGAAGGTCACC
ATTTCCTGCTCTGGAAGCACCTCCAACATTGGGAATAATTATGTCTCCTGGTACCAACA
GCACCCAGGCAAAGCCCCCAAACTCATGATTTATGATGTCAGTAAGCGGCCCTCAGGG
GTCCCTGACCGATTCTCTGGCTCCAAGTCTGGCAACTCAGCCTCCCTGGACATCAGTG
GGCTCCAGTCTGAGGATGAGGCTGATTATTACTGTGCAGCATGGGATGACAGCCTGAG
TGAATTTCTCTTCGGAACTAGGACCAAGCTGACCGTCCTA (SEQ ID NO: 107)
Her2_S1R3C1_CS_1D3
V_{H} with CDR1, CDR2 and CDR3 underlined
CAGGTGCAGCTGCAGGAGTCGGGTCCAGGACTGGTGAAGCCCTCGCAGACCTTGTCA
CTCACCTGTGGCATCTCCGGGGACAGTGTCTCTAGCAACAGTGCTGCTTGGAACTGGA
TCAGGCAGTCCCCAACGAGAGGCCTTGAGTGGCTGGGAAGGACATATTACAGGTCCAG
TTGGTATCATAACTATGCACCTTCTATGAACAGTCGATTAACCATCATCGCAGACACATC
CAAAAACCAGTTCTCTTTGCAACTGAACTCTGTGACTCCCGAGGACACGGCTGTATATT
ACTGTGCAAGCGGGTGGGCCTTTGATGTCTGGGGCAGGGGAACCCTGGTCACCGTCT
CGAGT (SEQ ID NO: 108)
Her2_S1R3C1_CS_1D3
V_{L} with CDR1, CDR2 and CDR3 underlined
CAGTCTGTGCTGACTCAGCCACCCTCCGCGTCCGGGTCTCCTGGACAGTCAGTCACCA
TCTCCTGCACTGGAACCAGCAGTGACGTTGGTGCTTATGACTTTGTCTCCTGGTACCAA
CAGCACCCTGGCAAAGCCCCCAAACTCATGATTTATGAGGTCAATAAGCGGCCCTCAG
GGGTCCCTGATCGCTTCTCTGGCTCCAAGTCTGGCAACACGGCCTCCCTGACCGTCTC
TGGGCTCCAGGCTGAGGATGAGGCTGATTATTACTGCAGCTCATATGCAGGCAGCAAG
AATTTGCTTTTCGGCGGAGGGACCAAGCTGACCGTCCTA (SEQ ID NO: 109)
Her2_S1R3B2_DP47_1E8
V_{H} with CDR1, CDR2 and CDR3 underlined
GAGGTGCAGCTGTTGGAGTCTGGGGGAGGCTTGGTACAGCCTGGGGGGTCCCTGAGA
CTCTCCTGTGCAGCCTCTGGATTCACCTTTAGCAGCTATGCCATGAGCTGGGTCCGCC
AGGCTCCAGGGAAGGGGCTGGAGTGGGTCTCAGCTATTAGTGGTAGTGGTGGTAGCA
CATACTACGCAGACTCCGTGAAGGGCCGGTTCACCATCTCCAGAGACAATTCCAAGAA
CACGCTGTATCTGCAAATGAACAGCCTGAGAGCCGAGGACACGGCCGTGTATTACTGT
GCGAGACAGTCGGGCGCGGACTGGTACTTCGATCTCTGGGGCCGAGGCACCCTGGTC
ACCGTCTCGAGT (SEQ ID NO: 110)
Her2_S1R3B2_DP47_1E8
V_{L} with CDR1, CDR2 and CDR3 underlined
CAGGCTGTGCTGACTCAGCCGTCCGCAGTTTCTGGGGCCCCAGGGCAGAGGGTCACC
ATCTCCTGCACTGGGACCAGCTCCAACATCGGGACAAACTATCTTGTACACTGGTATCA
GCAACGTCCAGGAACAGCCCCCCAACTCCTCGTCTCTGGTAACAACACTCGACCCTCT
GGGGTCACTGACCGGTTCTCTGTCTCCAAGTCTGCCACTTCAGCCTCCCTGGCCATCA
CTGGGCTCCAGGCTGAGGATGAGGCTGATTATTACTGCCAGACCTATGACATCAACTT
GAGGGTTTGGGTGTTCGGCGGAGGGACCAAGGTCACCGTCCTA (SEQ ID NO: 111)
Her2_S1R3B2_BMV_1G2
V_{H} with CDR1, CDR2 and CDR3 underlined
CAGGTGCAGCTGGTGCAGTCTGGAGCTGAGGTGAAGAAGCCTGGGTCCTCGGTGAAG
GTCTCCTGCAAGGCTTCTGGTTACACCTTTACCAGCTATGGTATCAGCTGGGTGCGACA
GGCCCCTGGACAAGGGCTTGAGTGGATGGGATGGATCAGCGCTTACAATGGTAACACA
AACTATGCACAGAAGCTCCAGGGCAGAGTCACCATGACCACAGACACATCCACGAGCA
CAGCCTACATGGAGCTGAGGAGCCTGAGATCTGACGACACGGCCGTGTATTACTGTGC
GAGAGTCCCGGGCGTAAGTGGGAGCTATCCAGACTACTACTACATGGACGTCTGGGG
CAAGGGAACCCTGGTCACCGTCTCCTCA (SEQ ID NO: 112)
Her2_S1R3B2_BMV_1G2
V_{L} with CDR1, CDR2 and CDR3 underlined
GACATCCAGATGACCCAGTCTCCTTCCACCCTGTCTGCATCTATTGGAGACAGAGTCAC
CATCACCTGCCGGGCCAGTGAGGGTATTTATCACTGGTTGGCCTGGTATCAGCAGAAG
CCAGGGAAAGCTCCTAAACTCCTGATCTATAAGGCCTCTAGTTTAGCCAGTGGGGCCC
CATCAAGGTTCAGCGGCAGTGGATCTGGGACAGATTTCACTCTCACCATCAGCAGCCT
GCAGCCTGATGATTTTGCAACTTATTACTGCCAACAATATAGTAATTATCCGCTCACTTT
CGGCGGAGGGACCAAGCTGGAGATCAAA (SEQ ID NO: 113)
Her2_S1R3B2_BMV_1H5
V_{H} with CDR1, CDR2 and CDR3 underlined
GAGGTGCAGCTGGTGCAGTCTGGGGGAGGCTTGGTCAGGCCTGGAGGGTCCCTGAGA
CTCTCCTGTGCAGCCTCGGGATTCTCCTTCAGTGACTACTACATGACCTGGATCCGCCA
GATTCCAGGGAAGGGGCTGGAGTGGGTGGCAGTTATATGGAATGATGGAAGTGATAGA
TACTATGCAGACTCCGTGAAGGGCCGATTCACCATTTCCAGAGACAATTCCAAGAACAC
GCTGTTTCTGCAAATGAGCAGCCTGAGAGACGAGGACACGGCTCTATATTACTGTGTG
AGAGGGGGACCAACAGCTTCAAGCGGATTTGACTACTGGGGCCGAGGCACCCTGGTC
ACCGTCTCGAG (SEQ ID NO: 114)
Her2_S1R3B2_BMV_1H5
V_{L} with CDR1, CDR2 and CDR3 underlined
TCGTCTGAGCTGACTCAGCCTGCCTCCGTGTCTGGGTCTCCTGGACAGTCGATCACCA
TCTCCTGCACTGGAACCAGCAGTGACGTTGGTGGTTATAACTATGTCTCCTGGTACCTA
CAACACCCAGGCAAAGCCCCCAAACTCATGATTTATGAGGGCAGTAAGCGGCCCTCAG
GGGTTTCTAATCGCTTCTCTGGCTCCAAGTCTGGCAACACGGCCTCCCTGACAATCTCT
GGGCTCCAGGCTGAGGACGAGGCTGATTATTACTGCAGCTCATATACAACCAGGAGCA
CTCGAGTTTTCGGCGGAGGGACCAAGCTGACCGTCCTA (SEQ ID NO: 115)
Her2_S1R3C1_CS_1A6
V_{H} with CDR1, CDR2 and CDR3 underlined
GAGGTGCAGCTGGTGCAGTCTGGGGCAGAGGTGAAAAAGCCCGGGGAGTCTCTGAAG
ATCTCCTGTAAGGGTTTTGGATACAATTTTCGCAGCGCCTGGATCGGCTGGGTGCGCC
AGATGCCCGGCAAAGGCCTGGAGTGGATGGGGGTCATCTATCCTGGTGACTCTGATGT
CAGATACAGTCCGTCCTTCCAAGGCCAGGTCACCATCTCAGCCGACAAGTCCATCAGT
ACCGCCTACCTGCAGTGGAGCAGCCTGAAAGCCTCGGACACCGCCATGTATTATTGTA
CGAGACCCGTAGGGCAGTGGGTGGACTCTGACTATTGGGGCAAGGGAACCCTGGTCA
CCGTCTCGAGT (SEQ ID NO: 116)
Her2_S1R3C1_CS_1A6
V_{L} with CDR1, CDR2 and CDR3 underlined
CAGTCTGTGTTGACGCAGCCGCCCTCAGCGTCTGGGACCCCCGGACAGAGGGTCACC
ATCTCTTGTTCTGGAAGCAGCTCCAACATCGGAACTAATACTGTGAACTGGTACCAGCA
GCTTCCAGGAACGGCCCCCAAACTCCTCATCTATACTAGTAATCAGCGGCCCTCAGGG
GTCCCTGCCCGCTTCTCTGCCTCCAACTCTGGCACCTCAGCCTCCCTGGCCATCAGTG
GGCTCCGGTCCGAGGATGAGGCTGATTATTATTGTGCAGCGTGGGATGACAAGTTGAG
TGGTGCGGTGTTCGGCGGAGGGACCAAGCTGACCGTCCTA (SEQ ID NO: 117)
Her2_S1R3B2_DP47_1C9
V_{H} with CDR1, CDR2 and CDR3 underlined
GAGGTGCAGCTGTTGGAGTCTGGGGGAGGCTTGGTACAGCCTGGGGGGTCCCTGAGA
CTCTCCTGTGCAGCCTCTGGATTCACCTTTAGCAGCTATGCCATGAGCTGGGTCCGCC
AGGCTCCAGGGAAGGGGCTGGAGTGGGTCTCAGCTATTAGTGGTAGTGGTGGTAGCA
CATACTACGCAGACTCCGTGAAGGGCCGGTTCACCATCTCCAGAGACAATTCCAAGAA
CACGCTGTATCTGCAAATGAACAGCCTGAGAGCCGAGGACACGGCCGTGTATTACTGT
GCGAGATGGAGGCCTCTTCTAGACTACCACTTTGACCAATGGGGCCAAGGGACAATGG
TCACCGTCTCGAGT (SEQ ID NO: 118)
Her2_S1R3B2_DP47_1C9
V_{L} with CDR1, CDR2 and CDR3 underlined
CAGTCTGTGCTGACTCAGCCACCCTCAGCGTCTGGGACCCCCGGACAGACGGTAACAA
TCTCTTGTTCTGGAAGCAGCTCCAACATCGGAAGTAGTGTTGTTAATTGGTACCAGCAG
TTCCCAGGAACGGCCCCCAAAGTCCTCGTCTATAGTAACACTCAGCGGCCCTCAGGGG
TCCCTGACCGATTCTCTGGCTCCAGGTCTGGCACCTCAGCCTCCCTGGCCATCAGTGG
GCTCCAGTCTGAGGATGAGGCTGATTATTACTGTTTAGCATGGGATGCCAGCCTGAATG
GTTGGGTGTTCGGCGGAGGGACCAAGCTGACCGTCCTA (SEQ ID NO: 119)
Her2_S1R3B2_DP47_1E10
V_{H} with CDR1, CDR2 and CDR3 underlined
GAGGTGCAGCTGTTGGAGTCTGGGGGAGGCTTGGTACAGCCTGGGGGGTCCCTGAGA
CTCTCCTGTGCAGCCTCTGGATTCACCTTTAGCAGCTATGCCATGAGCTGGGTCCGCC
AGGCTCCAGGGAAGGGGCTGGAGTGGGTCTCAGCTATTAGTGGTAGTGGTGGTAGCA
CATACTACGCAGACTCCGTGAAGGGCCGGTTCACCATCTCCAGAGACAATTCCAAGAA
CACGCTGTATCTGCAAATGAACAGCCTGAGAGCCGAGGACACGGCCGTGTATTACTGT
GCGAGAGGATACAGTGGCTACGATGACCCTGACTCCTGGGGGAGAGGGACCACGGTC
ACCGTCTCGAGT (SEQ ID NO: 120)
Her2_S1R3B2_DP47_1E10
V_{L} with CDR1, CDR2 and CDR3 underlined
CACGTTATACTGACTCAACCGCCCTCAACGTCTGGGACCCCCGGGCAGACGGTCACCA
TCTCTTGTTCTGGGAGCAGCTCCAACATCGGAAGTCATTATGTATACTGGTACCAGCAG
CTCCCAGGAACGGCCCCCAAACTCCTCATCTATAGGAATAATCAGCGGCCCTCAGGGG
TCCCTGACCGATTCTCTGGCTCCAAGTCTGGCACCTCAGCCTCCCTGGCCATCAGTGG
GCTCCGGTCCGAGGATGAGACTGATTATTACTGTGCAGCATGGGATGACAGCCTGAGT
GGTCGAGTCTTCGGAACTGGGACCAAGCTGACCGTCCTA (SEQ ID NO: 121)
Her2_S1R3C1_CS_1B10
V_{H} with CDR1, CDR2 and CDR3 underlined
CAGGTACAGCTGCAGCAGTCAGGGGCTGAGGTGAAGAAGCCTGGGTCCTCGGTGAAG
GTCTCCTGCAAGGCTTCTGGAGGCACCATCAGCAACTATGCTATCAGTTGGGTGCGGC
TGGCCCCTGGACAAGGTCTTGAGTGGATGGGAAGTATCGTCCCTCTTCATGGGACAAC
AAACTTCGCACAGAAATTCCAGGGCAGAGTCACGATCACCGCGGACGAGTCCACGAGC
ACATCCTACATGGAGGTGAACGTCCTGACATATGAAGACACGGCGATGTATTATTGTGC
GTCTCTCAATTGGGGCTACTGGGGCCGGGGCACCCTGGTCACCGTCTCGAGT (SEQ ID NO: 122)
Her2_S1R3C1_CS_1B10
V_{L} with CDR1, CDR2 and CDR3 underlined
AATTTTATGCTGACTCAGCCCCACTCTGTGTCGGAGTCTCCGGGGAAGACGGTAACCA
TCTCCTGCACCGGCAGTAGTGGCAGCATTGCCAGCAACTATGTGCAGTGGTACCAGCA
GCGCCCGGACAGTGCCCCCACCACTGTGATCTATGAGGATAATCGAAGATCCTCTGGA
GTCCCTGATCGGTTCTCTGGCTCCATCGACAGCTCCTCCAACTCTGCCTCCCTCAGCAT
CTCTGGACTGAAGACTGAGGACGAGGCTGACTACTACTGTCAGTCCTATGATAGTAGC
GGTCATGTGGTCTTCGGCGGAGGGACCAAGCTGACCGTCCTA (SEQ ID NO: 123)
Her2_S1R3A1_BMV_1F3
V_{H} with CDR1, CDR2 and CDR3 underlined
GAGGTGCAGCTGGTGGAGTCTGGGGAAGGCCTGGTCAAGCCTGGGGGGTCCCTGAG
ACTCTCCTGTACAGCCTCTGGATTCACCTTCAGGAGTTATAGCTTGAACTGGGTCCGCC
AGGCTCCAGGGCAGGGGCTGGAGTGGGTCTCATCCATTAGTAGTACTAGTACTTACAT
ATACTACGCAGACTCGGTGAAGGGCCGATTCACCATCTCCAGAGACGACGCCAAGAAC
ACACTGTATCTGCAAATGAACAGCCTGAGAGCCGAAGACACAGCTGCATATTACTGTGT
TAGACTGGGATCTGGTGGGGGATATTTTCCTGACTACTGGGGCAGGGGCACCCTGGTC
ACCGTCTCGAGT (SEQ ID NO: 124)
Her2_S1R3A1_BMV_1F3
V_{L} with CDR1, CDR2 and CDR3 underlined
TCGTCTGAGCTGACTCAGGACCCTGCTGTGTCTGTGGCCTTGGGACAGACAGTCAGGA
TCACATGCCAAGGAGACAGCCTCAGAAGCTATTATGCAAGCTGGTACCAGCAGAAGCC
AGGACAGGCCCCTGTACTTGTCATCTATGGTAAAAACAACCGGCCCTCAGGGATCCCA
GACCGATTCTCTGGCTCCAGCTCAGGAAACACAGCTTCCTTGACCATCACTGGGGCTC
AGGCGGAAGATGAGGCTGACTATTACTGTAACTCCCGGGACAGCAGTGGTAACCATGT
GGTATTCGGCGGAGGGACCAAGCTGACCGTCCTA (SEQ ID NO: 125)
Her2_S1R3B1_BMV_1G11
V_{H} with CDR1, CDR2 and CDR3 underlined
CAGGTGCAGCTGGTGCAGTCTGGGGGAGGCTTGGTCCAGCCGGGGGGGTCCCTGAG
ACTCTCCTGTGCAGCCTCTGGATTCACGTTTAGTACCTATGCCATGAGTTGGGCCCGCC
AGGCTCCAGGGAAGGGGCTGGAGTGGGTCTCAAGTATTAGTGGTGATGGTGGAAGAA
TTCTCGATGCAGACTCCGCGAAGGGCCGGTTCACCATCTCCAGAGACAATTCCAAGAA
CACGCTGTATCTGCAAATGAACGGCCTGAGAGTCGAGGACACGGCCCTTTATTACTGT
GCGAGAGCGGACGGTAACTACTGGGGCAGGGGGACAATGGTCACCGTCTCTTCA (SEQ ID NO: 126)
Her2_S1R3B1_BMV_1G11
V_{L} with CDR1, CDR2 and CDR3 underlined
CAGTCTGTGCTGACTCAGCCTGCCTCCGTGTCTGGGTCTCCTGGACAGTCGATCACCA
TCTCCTGCACTGGAACCAGCAGTGACGTTGGTGGTTATAACTATGTCTCCTGGTACCAA
CAACACCCAGGCAAAGCCCCCAAACTCATGATTTATGAGGGCAGTAAGCGGCCCTCAG
GGGTTTCTAATCGCTTCTCTGGCTCCAAGTCTGGCAACACGGCCTCCCTGACAATCTCT
GGGCTCCAGGCTGAGGACGAGGCTGATTATTACTGCAGCTCATATACAACCAGGAGCA
CTCGAGTTTTCGGCGGAGGGACCAAGCTGACCGTCCTA (SEQ ID NO: 127)
Her2_S1R3A1_BMV_1G4
V_{H} with CDR1, CDR2 and CDR3 underlined
CAGGTGCAGCTGGTGGAGTCTGGGGCTGAGGTGAAGAAGCCTGGGGCCTCAGTGAAG
GTCTCCTGCAAGGCTTCTGGATACACCTTCACCAGTTATGATATCAACTGGGTGCGACA
GGCCCCCGGACAAAGGCTTGAGTGGATGGGATGGATCAACGCTGGCAATGGTAACAC
AAAATATTCACAGAAGTTCCAGGGCAGAGTCACCATTACCAGGGACACATCCGCGAGC
ACAGCCTACATGGAGCTGAGGAGCCTGAGATCTGACGACACGGCCGTGTATTACTGTG
CGAGAGGGAGGAGCTATGGCCACCCGTACTACTTTGACTACTGGGGCCAGGGAACCC
TGGTCACCGTCTCGAGT (SEQ ID NO: 128)
Her2_S1R3A1_BMV_1G4
V_{L} with CDR1, CDR2 and CDR3 underlined
CAGTCTGTGCTGACTCAGCCTGCCTCCGTGTCTGGGTCTCCTGGACAGTCGATCACCA
TCTCCTGCACTGGAACCAGCAGTGACGTTGGTGGTTATAACTATGTCTCCTGGTACCAA
CAACACCCAGGCAAAGCCCCCAAACTCATGATTTATGAGGGCAGTAAGCGGCCCTCAG
GGGTTTCTAATCGCTTCTCTGGCTCCAAGTCTGGCAACACGGCCTCCCTGACAATCTCT
GGGCTCCAGGCTGAGGACGAGGCTGATTATTACTGCAGCTCATATACAACCAGGAGCA
CTCGAGTTTTCGGCGGAGGGACCAAGCTGACCGTCCTA (SEQ ID NO: 129)
Her2_S1R3B1_BMV_1H11
V_{H} with CDR1, CDR2 and CDR3 underlined
GAGGTGCAGCTGGTGCAGTCTGGGGGAGGCCTGGTCAAGCCTGGGGGGTCCCTGAG
ACTCTCCTGTGCAGCGTCTGGATTCACCTTCAGTAGCTATGGGATGCACTGGGTCCGC
CAGGCTCCAGGCAAGGGGCTGGAGTGGGTGGCAGGTATTTTTTATGATGGAGGTAATA
AATACTATGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAGAGATAGGGGCTACTACTACATGGACGTCTGGGGCAAAGGGACCACGGTCACCG
TCTCCTCA (SEQ ID NO: 130)
Her2_S1R3B1_BMV_1H11
V_{L} with CDR1, CDR2 and CDR3 underlined
CAGTCTGTGTTGACGCAGCCGCCCTCAGTGTCTGGGGCCCCAGGACAGAGGGTCACC
ATCTCCTGCACTGGGAGAAGCTCCAACATCGGGGCGGGTCATGATGTACACTGGTACC
AGCAACTTCCAGGAACAGCCCCCAAACTCCTCATCTATGGTGACAGCAATCGGCCCTC
AGGGGTCCCTGACCGATTCTCTGGCTCCAGGTCTGGCACCTCAGCCTCCCTGGCCATC
ACTGGGCTCCAGGCTGAAGATGAGGCTGATTATTACTGCCAGTCCTATGACAGCAGCC
TGAGGGGTTCGGTATTCGGCGGAGGGACCAAGGTCACCGTCCTA (SEQ ID NO: 131)
Her2_S1R3A1_CS_1B9
V_{H} with CDR1, CDR2 and CDR3 underlined
AAGGTGCAGCTGGTGCAGTCTGGGACAGAGGTGAAAAAGCCCGGGGAGTCTCTGAAG
ATCTCCTGTCAGGGTTCTGGATACAGGTTTAGTAGTGACTGGATTGCCTGGGTGCGCC
AGATGCCCGGGAAAGGCCTGGAGTGGATGGGGATTGTCTATCCTGGTGACTCTGATAC
CAGATATAGCCCGTCCTTCCAAGGCCAAGTCACCATCTCAGCCGACAAGTCCATCAGTA
CTGCCTACCTGCAGTGGAGCGGCCTGAAGGCCTCGGACACCGCCAAGTATTACTGTGC
GAGAGTGCAACAGGCAGTGGGAGCTAAAGGTTATGCTATGGACGTCTGGGGCAAGGG
AACCCTGGTCACCGTCTCGAGT (SEQ ID NO: 132)
Her2_S1R3A1_CS_1B9
V_{L} with CDR1, CDR2 and CDR3 underlined
CAGACTGTGGTGATCCAGGAGCCATCGTTCTCAGTGTCCCCTGGAGGGACAGTCACAC
TCACTTGTGGCTTGAGCTCTGGCTCAGTCTCTACCAGTTACTACCCCAGCTGGTACCGG
CAGACCCCAGGCCAGGCTCCACACACACTCATTCACAACACAAAGATTCGCTCCTCTG
GGGTCCCTGATCGCTTCTCTGGCTCCATCCTTGGGAACAATGCTGCCCTCACCATCAC
GGGGGCCCAGGCAGATGATGAATCTGATTATTACTGTCTTTTGTATATGGGTAGCGGCA
TTTACGTGTTCGGCGGAGGGACCAAGCTGACCGTCCTA (SEQ ID NO: 133)
Her2_S1R3B1_BMV_1H9
V_{H} with CDR1, CDR2 and CDR3 underlined
CAGGTGCAGCTGCAGGAGTCGGGCGCAGGACTGGTGAAGCCTTCGGGGACCCTGTCC
CTCACCTGCGCTGTCTCTGGTGGCTCCATCAGCAGTGGTAACTGGTGGAGTTGGGTCC
GCCAGCCCCCAGGGAAGGGGCTGGAGTGGATTGGGGAAATCTCTCATAGTGGGAGCA
CCAACTACAACCCGTCCCTCAAGAGTCGAGTCACCATATCAGTAGACAAGTCCAAGAAC
CAGTTCTCCCTGAACCTGAGTTCTGTGACCGCCGCAGACACGGCCGTGTATTACTGTG
CGAGAGTAAGGGGTACGGTGGGGGATACACGGGGACCTGACTACTGGGGCCAGGGA
ACCCTGGTCACCGTCTCGAGT (SEQ ID NO: 134)
Her2_S1R3B1_BMV_1H9
V_{L} with CDR1, CDR2 and CDR3 underlined
TCGTCTGAGCTGACTCAGGACCCTGCTGTGTCTGTGGCCTTGGGACAGACAGTCAGGA
TCACATGCCAAGGAGACAGCCTCAGAAGCTATTATGCAAGCTGGTACCAGCAGAAGCC
AGGACAGGCCCCTGTACTTGTCATCTATGGTAAAAACAACCGGCCCTCAGGGATCCCA
GACCGATTCTCTGGCTCCAGCTCAGGAAACACAGCTTCCTTGACCATCACTGGGGCTC
AGGCGGAAGATGAGGCTGACTATTACTGTAACTCCCGGGACAGCAGTGGTAACCATGT
GGTATTCGGCGGAGGGACCAAGCTGACCGTCCTA (SEQ ID NO: 135)
Her2_S1R3A1_CS_1B10
V_{H} with CDR1, CDR2 and CDR3 underlined
GAAGTGCAGCTGGTGCAGTCTGGGGCTGAGGTGAAGAAGCCTGGGGCCTCAGTGAGG
GTCTCCTGCAAGGGTTCTGGAAACACCTTCACCGGCCACTACATCCACTGGGTGCGAC
AGGCCCCTGGACAAGGACTTGAGTGGCTGGGATGGATCGACCCTAACACTGGTGACAT
ACAGTATTCAGAAAACTTTAAGGGCTCGGTCACCTTGACCAGGGACCCATCCATCAACT
CAGTCTTCATGGACCTGATCAGGCTGACATCTGACGACACGGCCATGTATTACTGTGC
GAGAGAAGGTGCCGGGCTCGCCAACTACTATTACTACGGTCTGGACGTCTGGGGCCG
AGGGACAATGGTCACCGTCTCGAGT (SEQ ID NO: 136)
Her2_S1R3A1_CS_1B10
V_{L} with CDR1, CDR2 and CDR3 underlined
CAGACTGTGGTGCTCCAGGAGCCTTCGTTCTCAGTGTCCCCTGGGGGGACAGTCACAC
TCACTTGTGGCTTGAACTTTGGCTCAGTCTCTACTGCTTACTACCCCAGTTGGTACCAG
CAGACCCCAGGCCAAGCTCCACGCACGCTCATCTACGGCACAAATATTCGTTCCTCTG
GGGTCCCGGATCGCTTCTCTGGCTCCATCGTAGGGAACAAAGCTGCCCTCACCATCAC
GGGGGCCCAGACAGAAGATGAGTCTGATTATTATTGTGCGCTGTATATGGGTAGTGGC
ATGCTCTTCGGCGGCGGGACCAAGGTCACCGTCCTA (SEQ ID NO: 137)
Her2_S1R3B1_BMV_1C12
V_{H} with CDR1, CDR2 and CDR3 underlined
GAGGTGCAGCTGGTGCAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGC
CAGGCTCCAGGCAAGGGGCTGGAGTGGGTGGCAGTTATATCATATGATGGAAGTATTA
AATACTATGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGCGAACTGGTGAATATAGTGGCTACGATACGAGTGGTTACAGCAATTGGGGCCAAGG
CACCCTGGTCACCGTCTCGAGT (SEQ ID NO: 138)
Her2_S1R3B1_BMV_1C12
V_{L} with CDR1, CDR2 and CDR3 underlined
CAGTCTGTGCTGACTCAGCCACCCTCAGCGTCTGGGACCCCCGGGCAGAGGGTCACC
ATCTCTTGTTCTGGAAGCAGCTCCAACATCGGGAGTAACACTGTAAACTGGTACCAGCG
ACTCCCAGGAGCGGCCCCCCAACTCCTCATCTACAATAATGACCAGCGGCCCTCAGGG
ATCCCTGACCGATTCTCTGGCTCCAAGTCTGGCACCTCAGGCTCCCTGGTCATCAGTG
GGCTCCAGTCTGAAGATGAGGCTGATTACTACTGTGCGTCATGGGATGACAGTCTGAA
TGGTCGGGTGTTCGGCGGAGGGACCAAGCTGACCGTCCTA (SEQ ID NO: 139)
Her2_S1R3C1_BMV_1H11
V_{H} with CDR1, CDR2 and CDR3 underlined
GGGGTGCAGCTGGTGGAGTCTGGGGGAGGCCTGGTCAAGCCTGGGGGGTCCCTGAG
ACTCTCCTGTGCAGCCTCTGGATTCACCTTCAGTAGCTATAACATGAACTGGGTCCGCC
AGGCTCCAGGGAAGGGACTGGAGTGGGTCTCAGCTATTAGTGGTAGTGGTGGTAGCA
CATACTACGCAGACTCCGTGACGGGCCGGTTCACCATCTCCAGAGACAATTCCAAGAA
CACGCTGTATCTGCAAATGAACAGCCTGAGAGCCGAGGACACGGCCGTATATTACTGT
GCGAAAGATACCAGTGGCTGGTACGGGGACGGTATGGACGTCTGGGGCCGGGGAACC
CTGGTCACCGTCTCGAGT (SEQ ID NO: 140)
Her2_S1R3C1_BMV_1H11
V_{L} with CDR1, CDR2 and CDR3 underlined
GACATCCAGATGACCCAGTCTCCTTCCACCCTGTCTGCATCTATTGGAGACAGAGTCAC
CATCACCTGCCGGGCCAGTGAGGGTATTTATCACTGGTTGGCCTGGTATCAGCAGAAG
CCAGGGAAAGCCCCTAAACTCCTGATCTATAAGGCCTCTAGTTTAGCCAGTGGGGCCC
CATCAAGGTTCAGCGGCAGTGGATCAGGGACAGATTTCACTCTCACCATCAGCAGCCT
GCAGCCTGATGATTTTGCAACTTATTACTGCCAACAATATAGTAATTATCCGCTCACTTT
CGGCGGAGGGACCAAGCTGGAGATCAAA (SEQ ID NO: 141)
Her2_S1R3B1_BMV_1A10
V_{H} with CDR1, CDR2 and CDR3 underlined
CAGATGCAGCTGGTGCAGTCTGGGGGAGGCGTGGTCCAGCCTGGGAGGTCCCTGAGA
CTCTCCTGTGCAGCCTCTGGATTCACCTTCAGTAGCTATGGCATGCACTGGGTCCGCC
AGGCTCCAGGCAAGGGGCTGGAGTGGGTGGCAGTTATATCATATGATGGAAGTATTAA
ATACTATGCAGACTCCGTGAAGGGCCGATTCACCATCTCCAGAGACAATTCCAAGAACA
CACTGTATCTACAAATGAACAGCCTGAGAGCCGAGGACACGGGCGTTTATTACTGTTCG
AAAGATCGCTATAGCAGTGGCTGGTACAGCTCCGATGCTTTTGATATTTGGGGCCGAG
GGACAATGGTCACCGTCTCGAGT (SEQ ID NO: 142)
Her2_S1R3B1_BMV_1A10
V_{L} with CDR1, CDR2 and CDR3 underlined
TCTGAGCTGACTCAGGACCCTGCTGTGTCTGTGGCCTTGGGACAGACAGTCAGGATCA
CATGCCAAGGAGACAGCCTCAGAAGCTATTATGCAAGCTGGTACCAGCAGAAGCCAGG
ACAGGCCCCTGTACTTGTCATCTATGGTAAAAACAACCGGCCCTCAGGGATCCCAGAC
CGATTCTCTGGCTCCAGCTCAGGAAACACAGCTTCCTTGACCATCACTGGGGCTCAGG
CGGAAGATGAGGCTGACTATTACTGTCATTCCCGGGACAGCAGTGGTAACCATGTGCT
TTTCGGCGGAGGGACCAAGCTGACCGTCCTA (SEQ ID NO: 143)
Her2_S1R3A1_CS_1D11
V_{H} with CDR1, CDR2 and CDR3 underlined
GAGGTGCAGCTGGTGCAGTCTGGGGCAGAGGTGAAAAAGCCCGGAGAGTCTCTGAAG
ATCTCCTGTAAGGGCTCTGGATACACCTTTACCAACCACTGGATCGCCTGGGTGCGCC
AGATGCCCGGGAAAGGCCTGGAGTGGATGGGCATCATCTATCCTGGTGACTCTGAAAC
GAGGTACAGCCCGTCCTTCCAAGGCCACGTCACCATCTCAGCCGACAAGTCCATCAGT
ACCGCCTATTTGCAGTGGAGCACCCTGAAGGACTCGGACTCCGCCATGTACTTCTGTG
TGAGACAGGCCCGTGGCTGGGACGACGGACGGGCTGGATATTATTATTCCGGTATGGA
CGCCTGGGGCCAGGGAACCCTGGTCACCGTCTCGAGT (SEQ ID NO: 144)
Her2_S1R3A1_CS_1D11
V_{L} with CDR1, CDR2 and CDR3 underlined
CAGGCTGTGGTGCTCCAGGAGCCATCGTTCTCAGTGTCCCCTGGAGGGACAGTCACAC
TCACCTGTGGCTTGCGCTCTGGGTCAGTCTCTACTAGTCACTACCCCAGCTGGTACCA
GCAGACCCCAGGCCAGGCTCCACGCACGCTCATTTACAGCACAAACACTCGCTCTTCT
GGGGTCCCTGATCGCTTCTCTGGCTCCATCCTTGGGAACAAAGCTGCCCTCACCATCA
CGGGGGCCCAGGCAGATGATGAATCTAATTATTACTGTATGCTATACATGGGCAGTGG
CATGTATGTGTTCGGCGGAGGGACCAAGGTCACCGTCCTA (SEQ ID NO: 145)
Her2_S1R3C1_DP47_1H1
V_{H} with CDR1, CDR2 and CDR3 underlined
GAGGTGCAGCTGTTGGAGTCTGGGGGAGGCTTGGTACAGCCTGGGGGGTCCCTGAGA
CTCTCCTGTGCAGCCTCTGGATTCACCTTTAGCAGCTATGCCATGAGCTGGGTCCGCC
AGGCTCCAGGGAAGGGGCTGGAGTGGGTCTCAGCTATTAGTGGTAGTGGTGGTAGCA
CATACTACGCAGACTCCGTGAAGGGCCGGTTCACCATCTCCAGAGACAATTCCAAGAA
CACGCTGTATCTGCAAATGAACAGCCTGAGAGCCGAGGACACGGCCGTGTATTACTGT
GCGAGAGTCAGCGGGAGCCACTTTCCATTCTTTGACTCCTGGGGCCAGGGGACAATG
GTCACCGTCTCGAGT (SEQ ID NO: 146)
Her2_S1R3C1_DP47_1H1
V_{L} with CDR1, CDR2 and CDR3 underlined
CAGTCTGTGCTGACTCAGCCACCCTCGGTGTCAGTGGCCCCAGGACAGACGGCCAGA
ATTACCTGTGGGGGAGACAAGATTGGACATAAAAGTGTGCATTGGTATCAGCAGAAGC
CAGGCCAGGCCCCTGTGTTGCTCGTCTATGATGATAGGAAGCGGCCCTCAGGGATCCC
TGAGCGATTCTCTGGCTCCAACTCTGGGAACACGGCCACCCTGACCATCAGCAGGGTC
GAGGCCGGGGATGAGGCTGCCTATCACTGTCAGGTGTGGGATAGAAGTAGTGACCCTT
ATGTCTTCGGAACTGGGACCAAGGTCACCGTCCTA (SEQ ID NO: 147)
Her2_S1R3A1_CS_1B12
V_{H} with CDR1, CDR2 and CDR3 underlined
CAGGTGCAGCTGGTGCAATCTGGGGCTGAAGTGAAGAAGCCTGGGGCCTCAGTGAAG
GTCTCTTGTCAGGCTTCTGGATACACCTTCAGCGGGCACTATATGCACTTGGTGCGACA
GGCCCCTGGACAAGGGCTTGAGTGGATGGGGTGGATCCACCCTACCAGTGGTGGCAC
AACCTATGCACAGAAGTTTCAGGGCCGGGTCGTTATGACCAGGGACACGTCCATCAGC
ACAGCCTACATGGAACTGAGTAGGCTGACATCTGACGACACGGCCGTGTATTACTGTG
CAAGAATGTCCCAAAACTATGATGCTTTTGATATCTGGGGCCAAGGGACAATGGTCACC
GTCTCGAGT (SEQ ID NO: 148)
Her2_S1R3A1_CS_1B12
V_{L} with CDR1, CDR2 and CDR3 underlined
CAGGCTGTGCTGACTCAGCCGTCCTCAGTGTCTGGGGCCCCAGGGCAGAGGGTCACC
ATCTCCTGCACTGGGAGCAGCTCCAACATCGGGGCAGGTTATGATGTAAACTGGTACC
AACAATTTCCAGGAACAGCCCCCAAAATTATCGTCTATGGCGATCGGCCCTCAGGGGC
CCCTGACCGATTCTCTGGCTCCAAGTCTGGCACCTCAGCCTCCCTGGCAATCACTGGA
CTCCGGGCTGAGGATGAGGCTGATTATTACTGCCAGTCCTGGGACAGTCGCCTGAGTA
GTTATGTCTTCGGAACTGGGACCAAGGTCACCGTCCTA (SEQ ID NO: 149)
Her2_S1R3B1_BMV_1H5
V_{H} with CDR1, CDR2 and CDR3 underlined
CAGGTGCAGCTGCAGGAGTCGGGGGGAGGCGTGGTCCAGCCTGGGGGGTCCCTGAG
ACTCTCCTGTGCAGCGTCTGGATTCACCTTCAGTGGCTATGGCATGCACTGGGTCCGC
CAGGCTCCAGGCAAGGGGCTGGAGTGGGTGGCATCTGTACGGAACGATGGAAGTAAT
ACATACTACACAGACTCCGTGAAGGACCGATTCACCATCTCCAGAGACAACACCAAGAA
CACGCTGTATCTGCAAATGAACAGCCTGAGAGCCGAGGACACGGCCGTATATTACTGT
GCCAAGTCGAGAAGAGTGATGTATGGCACCTCCTATTACTTTGACTACTGGGGCAGAG
GCACCCTGGTCACCGTCTCCTCA (SEQ ID NO: 150)
Her2_S1R3B1_BMV_1H5
V_{L} with CDR1, CDR2 and CDR3 underlined
TCGTCTGAGCTGACTCAGGACCCTGCTGTGTCTGTGGCCTTGGGACAGACAGTCAGGA
TCACATGCCAAGGAGACAGCCTCAGAAGCTATTATGCAAGCTGGTACCAGCAGAAGCC
AGGACAGGCCCCTGTACTTGTCATCTATGGTAAAAACAACCGGCCCTCAGGGATCCCA
GACCGATTCTCTGGCTCCAGCTCAGGAAACACAGCTTCCTTGACCATCACTGGGGCTC
AGGCGGAAGATGAGGCTGACTATTACTGTAACTCCCGGGACAGCAGTGGTAACCATGT
GGTATTCGGCGGAGGGACCAAGCTGACCGTCCTA (SEQ ID NO: 151)
Her2_S1R3A1_DP47_1A6
V_{H} with CDR1, CDR2 and CDR3 underlined
GAGGTGCAGCTGTTGGAGTCTGGGGGAGGCTTGGTACAGCCTGGGGGGTCCCTGAGA
CTCTCCTGTGCAGCCTCTGGATTCACCTTTAGCAGCTATGCCATGAGCTGGGTCCGCC
AGGCTCCAGGGAAGGGGCTGGAGTGGGTCTCAGCTATTAGTGGTAGTGGTGGTAGCA
CATACTACGCAGACTCCGTGAAGGGCCGGTTCACCATCTCCAGAGACAATTCCAAGAA
CACGCTGTATCTGCAAATGAACAGCCTGAGAGCCGAGGACACGGCCGTGTATTACTGT
GCGAGAGATCTGGGAATAGACCCCCTTTGGAGTGGTTATTACACACCCCTTGACTATTG
GGGCCGAGGGACAATGGTCACCGTCTCGAGT (SEQ ID NO: 152)
Her2_S1R3A1_DP47_1A6
V_{L} with CDR1, CDR2 and CDR3 underlined
CACGTTATACTGACTCAACCGCCCTCAGCGTCTGGGACCCCCGGGCAGAGGGTCACC
ATCTCTTGTTCTGGAAGCAGCTCCAACATCGGAAGTAATTCCGTTAGCTGGTACCAGCA
GCTCCCAGGAACGGCCCCCAAACTCCTCATGTATACTAACAATCAGCGGCCCTCAGGG
GTCCCTGACCGATTCTCTGGCTCCAAGTCTGGCACCTCAGCCTCCCTGGCCATCAGTG
GGCTCCAGTCTGAGGATGAGGCTGATTATTACTGTGCGACATGGGATGCCAGCCTGAA
TACTTGGGTGTTCGGCGGAGGGACCAAGGTCACCGTCCTA (SEQ ID NO: 153)
Her2_S1R3B1_DP47_1E1
V_{H} with CDR1, CDR2 and CDR3 underlined
GAGGTGCAGCTGTTGGAGTCTGGGGGAGGCTTGGTACAGCCTGGGGGGTCCCTGAGA
CTCTCCTGTGCAGCCTCTGGATTCACCTTTAGCAGCTATGCCATGAGCTGGGTCCGCC
AGGCTCCAGGGAAGGGGCTGGAGTGGGTCTCAGCTATTAGTGGTAGTGGTGGTAGCA
CATACTACGCAGACTCCGTGAAGGGCCGGTTCACCATCTCCAGAGACAATTCCAAGAA
CACGCTGTATCTGCAAATGAACAGCCTGAGAGCCGAGGACACGGCCGTGTATTACTGT
GCGAGAGGCGGGAGTGGGAGTGACTACTGGGGCCAGGGGACAATGGTCACCGTCTC
GAGT (SEQ ID NO: 154)
Her2_S1R3B1_DP47_1E1
V_{L} with CDR1, CDR2 and CDR3 underlined
AATTTTATGCTGACTCAGCCCCACTCTGTGTCGGGGTCTCCGGGGAAGACGGTAACCA
TCTCCTGCACCCGCAGCAGTGGCTACATTGACAGCAAGTATGTGCAGTGGTACCAGCA
GCGCCCGGGCAGTGCCCCCACCACTGTGATCTATGAGGATAACCGAAGACCCTCTGG
GGTCCCTGATCGGTTCTCTGGCTCCATCGACAGCTCCTCCAACTCTGCCTCCCTCACC
ATCTCTGGACTGGAGACTGAGGACGAGGCTGACTATTACTGTCAGTCTTATGATGACAC
CAATGTGGTGTTCGGCGGAGGGACCAAGGTCACCGTCCTA (SEQ ID NO: 155)
Her2_S1R3B1_BMV_1A1
V_{H} with CDR1, CDR2 and CDR3 underlined
GAGGTCCAGCTGGTGCAGTCTGGAGCTGAGGTGAAGGAGCCTGGGGCCTCAGTGAAG
GTCTCCTGCAAGGCCTCTGGTTACGACTTTTCCAACTATGGTTTCAGCTGGGTGCGCCA
GGCCCCTGGACAAGGTCTTGAGTGGATGGGATGGATCAGCTCTTATAATGGTTACACA
AACTATGCACAGAGACTCCAGGGCAGAGTCACCATGACCACAGACACATCCACGAGCA
CAGCCTACATGGAGCTGAGGAGCCTGAGATCTGACGACACAGCTGTCTATTACTGTGC
GAGAGATCGAGGACTTGGAAACTGGTACTTCGATCTCTGGGGCCAAGGCACCCTGGTC
ACCGTCTCGAGT (SEQ ID NO: 156)
Her2_S1R3B1_BMV_1A1
V_{L} with CDR1, CDR2 and CDR3 underlined
CAGTCTGTGCTGACTCAGCCTGCCTCCGTGTCTGGGTCTCCTGGACAGTCGATCACCA
TCTCCTGCACTGGAACCAGCAGTGACGTTGGTGGTTATAACTATGTCTCCTGGTACCAA
CAACACCCAGGCAAAGCCCCCAAACTCATGATTTATGAGGGCAGTAAGCGGCCCTCAG
GGGTTTCTAATCGCTTCTCTGGCTCCAAGTCTGGCAACACGGCCTCCCTGACAATCTCT
GGGCTCCAGGCTGAGGACGAGGCTGATTATTACTGCAGCTCATATACAACCAGGAGCA
CTCGAGTTTTCGGCGGAGGGACCAAGCTGACCGTCCTA (SEQ ID NO: 157)
>HER018_CDS
atggattttcaagtgcagattttcagcttcctgctaatcagtgcttcagtcataatgtccagaggagatattcagatgacccagagcc
cgagcagcctgagcgcgagcgtgggcgatcgcgtgaccattacctgccgcgcgagccaggatgtgaacaccgcggtggcgt
ggtatcagcagaaaccgggcaaagcgccgaaactgctgatttatagcgcgagctttctgtatagcggcgtgccgagccgcttta
gcggcagccgcagcggcaccgattttaccctgaccattagcagcctgcagccggaagattttgcgacctattattgccagcagca
ttataccaccccgccgacctttggccagggcaccaaagtggaaattaaacgcaccgggggtggaggctctggtggcggtggct
ctggcggaggtggatccggtggcggcggatctgaagtgcagctggtggaaagcggcggcggcctggtgcagccgggcggca
gcctgcgcctgagctgcgcggcgagcggctttaacattaaagatacctatattcattgggtgcgccaggcgccgggcaaaggcc
tggaatgggtggcgcgcatttatccgaccaacggctatacccgctatgcggatagcgtgaaaggccgctttaccattagcgcgga
taccagcaaaaacaccgcgtatctgcagatgaacagcctgcgcgcggaagataccgcggtgtattattgcagccgctggggcg
gcgatggcttttatgcgatggattattggggccagggcaccctggtgaccgtgagcagtgatcaggagcccaaatcttgtgacaa
aactcacacatctccaccgtgctcagcacctgaactcctgggtggaccgtcagtcttcctcttccccccaaaacccaaggacacc
ctcatgatctcccggacccctgaggtcacatgcgtggtggtggacgtgagccacgaagaccctgaggtcaagttcaactggtac
gtggacggcgtggaggtgcataatgccaagacaaagccgcgggaggagcagtacaacagcacgtaccgtgtggtcagcgtc
ctcaccgtcctgcaccaggactggctgaatggcaaggagtacaagtgcaaggtctccaacaaagccctcccagcccccatcg
agaaaaccatctccaaagccaaagggcagccccgagaaccacaggtgtacaccctgcccccatcccgggatgagctgacc
aagaaccaggtcagcctgacctgcctggtcaaaggcttctatccaagcgacatcgccgtggagtgggagagcaatgggcagc
cggagaacaactacaagaccacgcctcccgtgctggactccgacggctccttcttcctctacagcaagctcaccgtggacaag
agcaggtggcagcaggggaacgtcttctcatgctccgtgatgcatgaggctctgcacaaccactacacgcagaagagcctctc
cctgtctccgggtaaatga (SEQ ID NO: 158)
>HER018_Protein_leader-stop
MDFQVQIFSFLLISASVIMSRGDIQMTQSPSSLSASVGDRVTITCRASQDVNTAVAWYQQKP
GKAPKLLIYSASFLYSGVPSRFSGSRSGTDFTLTISSLQPEDFATYYCQQHYTTPPTFGQGT
KVEIKRTGGGGSGGGGSGGGGSGGGGSEVQLVESGGGLVQPGGSLRLSCAASGFNIKDT
YIHWVRQAPGKGLEWVARIYPTNGYTRYADSVKGRFTISADTSKNTAYLQMNSLRAEDTAV
YYCSRWGGDGFYAMDYWGQGTLVTVSSDQEPKSCDKTHTSPPCSAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 159)
>HER018_2h7_Leader_CDS
atggattttcaagtgcagattttcagcttcctgctaatcagtgcttcagtcataatgtccagagga (SEQ ID NO: 160)
>HER018_2h7_Leader_Protein
MDFQVQIFSFLLISASVIMSRG (SEQ ID NO: 161)
>HER018_VL_CDS
Gatattcagatgacccagagcccgagcagcctgagcgcgagcgtgggcgatcgcgtgaccattacctgccgcgcgagccag
gatgtgaacaccgcggtggcgtggtatcagcagaaaccgggcaaagcgccgaaactgctgatttatagcgcgagctttctgtat
agcggcgtgccgagccgctttagcggcagccgcagcggcaccgattttaccctgaccattagcagcctgcagccggaagatttt
gcgacctattattgccagcagcattataccaccccgccgacctttggccagggcaccaaagtggaaattaaacgcacc (SEQ ID NO: 162)
>HER018_VL_Protein
DIQMTQSPSSLSASVGDRVTITCRASQDVNTAVAWYQQKPGKAPKLLIYSASFLYSGVPSR
FSGSRSGTDFTLTISSLQPEDFATYYCQQHYTTPPTFGQGTKVEIKRT (SEQ ID NO: 163)
>HER018_G4Sx4_Linker_CDS
gggggtggaggctctggtggcggtggctctggcggaggtggatccggtggcggcggatct(SEQ ID NO: 164)
>HER018_G4Sx4_Linker_Protein
GGGGSGGGGSGGGGSGGGGS (SEQ ID NO: 165)
>HER018_VH_CDS
gaagtgcagctggtggaaagcggcggcggcctggtgcagccgggcggcagcctgcgcctgagctgcgcggcgagcggcttt
aacattaaagatacctatattcattgggtgcgccaggcgccgggcaaaggcctggaatgggtggcgcgcatttatccgaccaac
ggctatacccgctatgcggatagcgtgaaaggccgctttaccattagcgcggataccagcaaaaacaccgcgtatctgcagatg
aacagcctgcgcgcggaagataccgcggtgtattattgcagccgctggggcggcgatggcttttatgcgatggattattggggcc
agggcaccctggtgaccgtgagcagt(SEQ ID NO: 166)
>HER018_VH_Protein
EVQLVESGGGLVQPGGSLRLSCAASGFNIKDTYIHWVRQAPGKGLEWVARIYPTNGYTRY
ADSVKGRFTISADTSKNTAYLQMNSLRAEDTAVYYCSRWGGDGFYAMDYWGQGTLVTVSS (SEQ ID NO: 167)
>HER018_CSCS_Hinge_CDS
gagcccaaatcttgtgacaaaactcacacatctccaccgtgctca (SEQ ID NO: 168)
>HER018_CSCS_Hinge_Protein
EPKSCDKTHTSPPCS (SEQ ID NO: 169)
>HER018_Fc_Stop_CDS
gcacctgaactcctgggtggaccgtcagtcttcctcttccccccaaaacccaaggacaccctcatgatctcccggacccctgagg
tcacatgcgtggtggtggacgtgagccacgaagaccctgaggtcaagttcaactggtacgtggacggcgtggaggtgcataatg
ccaagacaaagccgcgggaggagcagtacaacagcacgtaccgtgtggtcagcgtcctcaccgtcctgcaccaggactggct
gaatggcaaggagtacaagtgcaaggtctccaacaaagccctcccagcccccatcgagaaaaccatctccaaagccaaag
ggcagccccgagaaccacaggtgtacaccctgcccccatcccgggatgagctgaccaagaaccaggtcagcctgacctgcct
ggtcaaaggcttctatccaagcgacatcgccgtggagtgggagagcaatgggcagccggagaacaactacaagaccacgcc
tcccgtgctggactccgacggctccttcttcctctacagcaagctcaccgtggacaagagcaggtggcagcaggggaacgtcttc
tcatgctccgtgatgcatgaggctctgcacaaccactacacgcagaagagcctctccctgtctccgggtaaatga (SEQ ID NO: 170)
>HER018_Fc_Stop_Protein
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK (SEQ ID NO: 171)
>HER026_CDS
atggaagcaccagcgcagcttctcttcctcctgctactctggctcccagataccaccggtgaggtgcagctggtgcagtctgggtct
gaggtgaggaggcctgggtcctcggtgagggtctcctgcacggcttctggagacacctccagcagctttaccgtcaactggctgc
gacaggcccctggacaaggtcttgagtggatgggagggatcacccctatgtttggcactgcaaactacgcacagatgttcgagg
acagagtcacgataaccgcggacgaaatggaactgagtggcctgacatctgaggacacggccgtgtatttttgtgcgacaggc
ccctccgattacgtttgggggagttatcgtttccttgacacctgggggcgggggaccacggtcaccgtctcgagtggaggcggcg
gttcaggcggaggtggctctggcggtggcggaagtgcacaggctgtgctgactcagccgtcctcagtgtctgcggccccaggac
aggaggtctccatctcctgctctggagccagatccaacgttgggggtaattatgtttcctggtaccaacacctcccaggaacagcc
cccaaactcctcatttatgacaataataagcgaccctcagggatgcctgaccgattctctggctccaagtctggcacgtcagccac
cctgggcatcaccggagtccagactgaggacgaggccgattattactgcgcaacatgggatagcagcctgagcgctgtggtctt
cggcggagggaccaagctgaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcat
aatgccaagacaaagccgcgggaggagcagtacaacagcacgtaccgtgtggtcagcgtcctcaccgtcctgcaccaggac
tggctgaatggcaaggagtacaagtgcaaggtctccaacaaagccctcccagcccccatcgagaaaaccatctccaaagcca
aagggcagccccgagaaccacaggtgtacaccctgcccccatcccgggatgagctgaccaagaaccaggtcagcctgacct
gcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 172)
>HER026_Protein_leader-stop
MEAPAQLLFLLLLWLPDTTGEVQLVQSGSEVRRPGSSVRVSCTASGDTSSSFTVNWLRQA
PGQGLEWMGGITPMFGTANYAQMFEDRVTITADEMELSGLTSEDTAVYFCATGPSDYVW
GSYRFLDTWGRGTTVTVSSGGGGSGGGGSGGGGSAQAVLTQPSSVSAAPGQEVSISCS
GARSNVGGNYVSWYQHLPGTAPKLLIYDNNKRPSGMPDRFSGSKSGTSATLGITGVQTED
EADYYCATWDSSLSAVVFGGGTKLTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 173)
>HER027_CDS
atggaagcaccagcgcagcttctcttcctcctgctactctggctcccagataccaccggtcaggtgcagctggtgcagtctgggtct
gaggtgaggaggcctgggtcctcggtgaggatctcctgcacggcttctggagacacctccagcagctttaccgtcaactgggtgc
gacaggcccctggacaaggtcttgagtggatgggagggatcacccctatgtttggcactgcaaactacgcacaggtgttcgagg
acagagtcacaataatcgcggacgagatggaactgagtggcctgacatctgaggacacggccgtgtatttctgtgcgacaggc
ccctccgattacgtttgggggagttatcgtttccttgacaactggggcaggggcaccctggtcaccgtctcgagtggaggcggcgg
ttcaggcggaggtggctctggcggtggcggaagtgcacagtctgtgctgactcagccaccctcagtgtctgcggccccagggca
gaaggtcaccatctcctgctctggaggcaggtccagcattgggaataattatgtgtcctggtatcaacacctcccaggaacagcc
cccaaactcctcatctatgacaataatcagcgaccctcagggattcctgaccgattctctggctccaagtctggcacgtcagccac
cctgggcatcaccggactccagactggggacgaggccgattattactgcggaacatgggatagcagcctgagtgctgtggtgttt
ggcggagggaccaaggtcaccgtcctaggtgacgtacgcgagcccaaatcttctgacaaaactcacacatgcccaccgtgcc
cagcacctgaactcctgggtggaccgtcagtcttcctcttccccccaaaacccaaggacaccctcatgatctcccggacccctga
ggtcacatgcgtggtggtggacgtgagccacgaagaccctgaggtcaagttcaactggtacgtggacggcgtggaggtgcata
atgccaagacaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctccaaagcca
aagggcagccccgagaaccacaggtgtacaccctgcccccatcccgggatgagctgaccaagaaccaggtcagcctgacct
gcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 174)
>HER027_Protein_leader-stop
MEAPAQLLFLLLLWLPDTTGQVQLVQSGSEVRRPGSSVRISCTASGDTSSSFTVNWVRQA
PGQGLEWMGGITPMFGTANYAQVFEDRVTIIADEMELSGLTSEDTAVYFCATGPSDYVWG
SYRFLDNWGRGTLVTVSSGGGGSGGGGSGGGGSAQSVLTQPPSVSAAPGQKVTISCSG
GRSSIGNNYVSWYQHLPGTAPKLLIYDNNQRPSGIPDRFSGSKSGTSATLGITGLQTGDEA
DYYCGTWDSSLSAVVFGGGTKVTVLGDVREPKSSDKTHTCPPCPAPELLGGPSVFLFPPK
PKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVM
HEALHNHYTQKSLSLSPGK (SEQ ID NO: 175)
>HER028_CDS
atggaagcaccagcgcagcttctcttcctcctgctactctggctcccagataccaccggtgaagtgcagctggtgcagtctggggc
tgaggtgaagaagcctggggcctcagtgaaggtctcctgcaaggcttctgggtacagcttcaccgccttctatattcactgggtgcg
acaggcccctggacaaggccttgagtatttgggatggatcgaccctaatactggtgccacaaaatatgcacagcgctttcagggc
agggtcatcatgacctgggacacgtccatcaccacagccaccatggaactgagcaggctgacgtctgacgactcggccgtcta
ctactgtgtgagagatttgcgggagtggggctacgaattgtccgttgagtattggggcagaggaaccctggtcaccgtctcgagtg
gaggcggcggttcaggcggaggtggctctggcggtggcggaagtgcacagtctgtgctgactcagccaccctcagcgtctggg
acccccgggcagagggtcaccatctcttgttctggaagcagctccaacatcggaagtaattatgtatactggtaccagcagctcc
caggaacggcccccaaactcctcatctataggaataatcagcggccctcaggggtccctgaccgattctctggctccaagtctgg
cacctcagcctccctggccatcagtgggctccggtccgaggatgaggctgattattactgtgcagcatgggatgacagcctgagt
ggttgggtgttcggcggagggaccaagctgaccgtcctaggtgacgtacgcgagcccaaatcttctgacaaaactcacacatgc
ccaccgtgcccagcacctgaactcctgggtggaccgtcagtcttcctcttccccccaaaacccaaggacaccctcatgatctccc
ggacccctgaggtcacatgcgtggtggtggacgtgagccacgaagaccctgaggtcaagttcaactggtacgtggacggcgtg
gaggtgcataatgccaagacaaagccgcgggaggagcagtacaacagcacgtaccgtgtggtcagcgtcctcaccgtcctgc
accaggactggctgaatggcaaggagtacaagtgcaaggtctccaacaaagccctcccagcccccatcgagaaaaccatctc
caaagccaaagggcagccccgagaaccacaggtgtacaccctgcccccatcccgggatgagctgaccaagaaccaggtca
gcctgacctgcctggtcaaaggcttctatccaagcgacatcgccgtggagtgggagagcaatgggcagccggagaacaacta
caagaccacgcctcccgtgctggactccgacggctccttcttcctctacagcaagctcaccgtggacaagagcaggtggcagc
aggggaacgtcttctcatgctccgtgatgcatgaggctctgcacaaccactacacgcagaagagcctctccctgtctccgggtaa
atga (SEQ ID NO: 176)
>HER028_Protein_leader-stop
MEAPAQLLFLLLLWLPDTTGEVQLVQSGAEVKKPGASVKVSCKASGYSFTAFYIHWVRQAP
GQGLEYLGWIDPNTGATKYAQRFQGRVIMTWDTSITTATMELSRLTSDDSAVYYCVRDLRE
WGYELSVEYWGRGTLVTVSSGGGGSGGGGSGGGGSAQSVLTQPPSASGTPGQRVTISC
SGSSSNIGSNYVYWYQQLPGTAPKLLIYRNNQRPSGVPDRFSGSKSGTSASLAISGLRSED
EADYYCAAWDDSLSGWVFGGGTKLTVLGDVREPKSSDKTHTCPPCPAPELLGGPSVFLFP
PKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCS
VMHEALHNHYTQKSLSLSPGK (SEQ ID NO: 177)
>HER029_CDS
atggaagcaccagcgcagcttctcttcctcctgctactctggctcccagataccaccggtgaggtgcagctggtgcagtctggggc
tgaggtgaagaagcctggggcctcagtgaaggtctcctgcaaggcttctggatacaccttcaccggctactatatgcactgggtgc
gacaggcccctggacaagggcttgagtggatgggatggatcaaccctaacagtggtggcacaaactatgcacagaagtttcag
ggctgggtcaccatgaccagggacacgtccatcagcacagcctacatggagctgagcaggctgagatctgacgacacggcc
gtgtattactgtgcgagagattctactatggccccaggtgcttttgatatctggggccgaggcaccctggtcaccgtctcgagtgga
ggcggcggttcaggcggaggtggctctggcggtggcggaagtgcacagtctgtgctgactcagccaccctcggtgtcagtggcc
ccaggacagacggccaggatgacctgtgggggaaacaacattgaaagtaaaactgtgcattggtaccagcagaagccgggc
caggcccctgtgctggtcgtctacaatgataacgtccggccctcagggatccctgcgcgattctctggctccaactccggcaacac
ggccaccctgaccatcaacagggtcgaagccggggatgaggccgactattattgtcaggtgtgggactccagtagagatcaag
gggtattcggcggagggaccaagctgaccgtcctaggtgacgtacgcgagcccaaatcttctgacaaaactcacacatgccca
ccgtgcccagcacctgaactcctgggtggaccgtcagtcttcctcttccccccaaaacccaaggacaccctcatgatctcccgga
cccctgaggtcacatgcgtggtggtggacgtgagccacgaagaccctgaggtcaagttcaactggtacgtggacggcgtggag
gtgcataatgccaagacaaagccgcgggaggagcagtacaacagcacgtaccgtgtggtcagcgtcctcaccgtcctgcacc
aggactggctgaatggcaaggagtacaagtgcaaggtctccaacaaagccctcccagcccccatcgagaaaaccatctcca
aagccaaagggcagccccgagaaccacaggtgtacaccctgcccccatcccgggatgagctgaccaagaaccaggtcagc
ctgacctgcctggtcaaaggcttctatccaagcgacatcgccgtggagtgggagagcaatgggcagccggagaacaactaca
agaccacgcctcccgtgctggactccgacggctccttcttcctctacagcaagctcaccgtggacaagagcaggtggcagcag
gggaacgtcttctcatgctccgtgatgcatgaggctctgcacaaccactacacgcagaagagcctctccctgtctccggg
taaatga (SEQ ID NO: 178)
>HER029_Protein_leader-stop
MEAPAQLLFLLLLWLPDTTGEVQLVQSGAEVKKPGASVKVSCKASGYTFTGYYMHWVRQA
PGQGLEWMGWINPNSGGTNYAQKFQGWVTMTRDTSISTAYMELSRLRSDDTAVYYCARD
STMAPGAFDIWGRGTLVTVSSGGGGSGGGGSGGGGSAQSVLTQPPSVSVAPGQTARMT
CGGNNIESKTVHWYQQKPGQAPVLVVYNDNVRPSGIPARFSGSNSGNTATLTINRVEAGD
EADYYCQVWDSSRDQGVFGGGTKLTVLGDVREPKSSDKTHTCPPCPAPELLGGPSVFLFP
PKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCS
VMHEALHNHYTQKSLSLSPGK (SEQ ID NO: 179)
>HER030_CDS
atggaagcaccagcgcagcttctcttcctcctgctactctggctcccagataccaccggtgaggtgcagctggtgcagtctggggg
aggcttggtcaggcctggagggtccctgagactctcctgtgcagcctcgggattctccttcagtgactactacatgacctggatccg
ccagattccagggaaggggctggagtgggtggcagttatatggaatgatggaagtgatagatactatgcagactccgtgaagg
gccgattcaccatttccagagacaattccaagaacacgctgtttctgcaaatgagcagcctgagagacgaggacacggctctat
attactgtgtgagagggggaccaacagcttcaagcggatttgactactggggccgaggcaccctggtcaccgtctcgagtggtg
gaggcggttcaggcggaggtggcagcggcggtggcggatcgtctgagctgactcagcctgcctccgtgtctgggtctcctggac
agtcgatcaccatctcctgcactggaaccagcagtgacgttggtggttataactatgtctcctggtacctacaacacccaggcaaa
gcccccaaactcatgatttatgagggcagtaagcggccctcaggggtttctaatcgcttctctggctccaagtctggcaacacggc
ctccctgacaatctctgggctccaggctgaggacgaggctgattattactgcagctcatatacaaccaggagcactcgagttttcg
gcggagggaccaagctgaccgtcctaggtgacgtacgcgagcccaaatcttctgacaaaactcacacatgcccaccgtgccc
agcacctgaactcctgggtggaccgtcagtcttcctcttccccccaaaacccaaggacaccctcatgatctcccggacccctgag
gtcacatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggatgagctgaccaagaaccaggtcagcctgacctgc
ctggtcaaaggcttctatccaagcgacatcgccgtggagtgggagagcaatgggcagccggagaacaactacaagaccacg
cctcccgtgctggactccgacggctccttcttcctctacagcaagctcaccgtggacaagagcaggtggcagcaggggaacgtc
ttctcatgctccgtgatgcatgaggctctgcacaaccactacacgcagaagagcctctccctgtctccgggtaaatga (SEQ ID NO: 180)
>HER030_Protein_leader-stop
MEAPAQLLFLLLLWLPDTTGEVQLVQSGGGLVRPGGSLRLSCAASGFSFSDYYMTWIRQIP
GKGLEWVAVIWNDGSDRYYADSVKGRFTISRDNSKNTLFLQMSSLRDEDTALYYCVRGGP
TASSGFDYWGRGTLVTVSSGGGGSGGGGSGGGGSSELTQPASVSGSPGQSITISCTGTS
SDVGGYNYVSWYLQHPGKAPKLMIYEGSKRPSGVSNRFSGSKSGNTASLTISGLQAEDEA
DYYCSSYTTRSTRVFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHE
ALHNHYTQKSLSLSPGK (SEQ ID NO: 181)
>HER031_CDS
atggaagcaccagcgcagcttctcttcctcctgctactctggctcccagataccaccggtcaggtgcagctgcaggagtcgggtc
caggactggtgaagccctcgcagaccttgtcactcacctgtggcatctccggggacagtgtctctagcaacagtgctgcttggaa
ctggatcaggcagtccccaacgagaggccttgagtggctgggaaggacatattacaggtccagttggtatcataactatgcacct
tctatgaacagtcgattaaccatcatcgcagacacatccaaaaaccagttctctttgcaactgaactctgtgactcccgaggacac
ggctgtatattactgtgcaagcgggtgggcctttgatgtctggggcaggggaaccctggtcaccgtctcgagtggaggcggcggtt
caggcggaggtggctctggcggtggcggaagtgcacagtctgtgctgactcagccaccctccgcgtccgggtctcctggacagt
cagtcaccatctcctgcactggaaccagcagtgacgttggtgcttatgactttgtctcctggtaccaacagcaccctggcaaagcc
cccaaactcatgatttatgaggtcaataagcggccctcaggggtccctgatcgcttctctggctccaagtctggcaacacggcctc
cctgaccgtctctgggctccaggctgaggatgaggctgattattactgcagctcatatgcaggcagcaagaatttgcttttcggcgg
agggaccaagctgaccgtcctaggtgacgtacgcgagcccaaatcttctgacaaaactcacacatgcccaccgtgcccagca
cctgaactcctgggtggaccgtcagtcttcctcttccccccaaaacccaaggacaccctcatgatctcccggacccctgaggtca
catgcgtggtggtggacgtgagccacgaagaccctgaggtcaagttcaactggtacgtggacggcgtggaggtgcataatgcc
aagacaaagccgcgggaggagcagtacaacagcacgtaccgtgtggtcagcgtcctcaccgtcctgcaccaggactggctg
aatggcaaggagtacaagtgcaaggtctccaacaaagccctcccagcccccatcgagaaaaccatctccaaagccaaagg
gcagccccgagaaccacaggtgtacaccctgcccccatcccgggatgagctgaccaagaaccaggtcagcctgacctgcctg
gtcaaaggcttctatccaagcgacatcgccgtggagtgggagagcaatgggcagccggagaacaactacaagaccacgcct
cccgtgctggactccgacggctccttcttcctctacagcaagctcaccgtggacaagagcaggtggcagcaggggaacgtcttct
catgctccgtgatgcatgaggctctgcacaaccactacacgcagaagagcctctccctgtctccgggtaaatga (SEQ ID NO: 182)
>HER031_Protein_leader-stop
MEAPAQLLFLLLLWLPDTTGQVQLQESGPGLVKPSQTLSLTCGISGDSVSSNSAAWNWIRQ
SPTRGLEWLGRTYYRSSWYHNYAPSMNSRLTIIADTSKNQFSLQLNSVTPEDTAVYYCASG
WAFDVWGRGTLVTVSSGGGGSGGGGSGGGGSAQSVLTQPPSASGSPGQSVTISCTGTS
SDVGAYDFVSWYQQHPGKAPKLMIYEVNKRPSGVPDRFSGSKSGNTASLTVSGLQAEDEA
DYYCSSYAGSKNLLFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHE
ALHNHYTQKSLSLSPGK (SEQ ID NO: 183)
>HER032_CDS
atggaagcaccagcgcagcttctcttcctcctgctactctggctcccagataccaccggtgaggtgcagctgttggagtctggggg
aggcttggtacagcctggggggtccctgagactctcctgtgcagcctctggattcacctttagcagctatgccatgagctgggtccg
ccaggctccagggaaggggctggagtgggtctcagctattagtggtagtggtggtagcacatactacgcagactccgtgaaggg
ccggttcaccatctccagagacaattccaagaacacgctgtatctgcaaatgaacagcctgagagccgaggacacggccgtgt
attactgtgcgagaggatacagtggctacgatgaccctgactcctgggggagagggaccacggtcaccgtctcgagtggaggc
ggcggttcaggcggaggtggctctggcggtggcggaagtgcacacgttatactgactcaaccgccctcaacgtctgggacccc
cgggcagacggtcaccatctcttgttctgggagcagctccaacatcggaagtcattatgtatactggtaccagcagctcccagga
acggcccccaaactcctcatctataggaataatcagcggccctcaggggtccctgaccgattctctggctccaagtctggcacctc
agcctccctggccatcagtgggctccggtccgaggatgagactgattattactgtgcagcatgggatgacagcctgagtggtcga
gtcttcggaactgggaccaagctgaccgtcctaggtgacgtacgcgagcccaaatcttctgacaaaactcacacatgcccaccg
tgcccagcacctgaactcctgggtggaccgtcagtcttcctcttccccccaaaacccaaggacaccctcatgatctcccggaccc
ctgaggtcacatgcgtggtggtggacgtgagccacgaagaccctgaggtcaagttcaactggtacgtggacggcgtggaggtg
cataatgccaagacaaagccgcgggaggagcagtacaacagcacgtaccgtgtggtcagcgtcctcaccgtcctgcaccag
gactggctgaatggcaaggagtacaagtgcaaggtctccaacaaagccctcccagcccccatcgagaaaaccatctccaaa
gccaaagggcagccccgagaaccacaggtgtacaccctgcccccatcccgggatgagctgaccaagaaccaggtcagcct
gacctgcctggtcaaaggcttctatccaagcgacatcgccgtggagtgggagagcaatgggcagccggagaacaactacaa
gaccacgcctcccgtgctggactccgacggctccttcttcctctacagcaagctcaccgtggacaagagcaggtggcagcagg
ggaacgtcttctcatgctccgtgatgcatgaggctctgcacaaccactacacgcagaagagcctctccctgtctccgggtaaatga (SEQ ID NO: 184)
>HER032_Protein_leader-stop
MEAPAQLLFLLLLWLPDTTGEVQLLESGGGLVQPGGSLRLSCAASGFTFSSYAMSWVRQA
PGKGLEWVSAISGSGGSTYYADSVKGRFTISRDNSKNTLYLQMNSLRAEDTAVYYCARGY
SGYDDPDSWGRGTTVTVSSGGGGSGGGGSGGGGSAHVILTQPPSTSGTPGQTVTISCSG
SSSNIGSHYVYWYQQLPGTAPKLLIYRNNQRPSGVPDRFSGSKSGTSASLAISGLRSEDET
DYYCAAWDDSLSGRVFGTGTKLTVLGDVREPKSSDKTHTCPPCPAPELLGGPSVFLFPPK
PKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVM
HEALHNHYTQKSLSLSPGK (SEQ ID NO: 185)
>HER033_CDS
atggaagcaccagcgcagcttctcttcctcctgctactctggctcccagataccaccggtcaggtacagctgcagcagtcagggg
ctgaggtgaagaagcctgggtcctcggtgaaggtctcctgcaaggcttctggaggcaccatcagcaactatgctatcagttgggt
gcggctggcccctggacaaggtcttgagtggatgggaagtatcgtccctcttcatgggacaacaaacttcgcacagaaattccag
ggcagagtcacgatcaccgcggacgagtccacgagcacatcctacatggaggtgaacgtcctgacatatgaagacacggcg
atgtattattgtgcgtctctcaattggggctactggggccggggcaccctggtcaccgtctcgagtggaggcggcggttcaggcgg
aggtggctctggcggtggcggaagtgcacttaattttatgctgactcagccccactctgtgtcggagtctccggggaagacggtaa
ccatctcctgcaccggcagtagtggcagcattgccagcaactatgtgcagtggtaccagcagcgcccggacagtgcccccacc
actgtgatctatgaggataatcgaagatcctctggagtccctgatcggttctctggctccatcgacagctcctccaactctgcctccct
cagcatctctggactgaagactgaggacgaggctgactactactgtcagtcctatgatagtagcggtcatgtggtcttcggcggag
ggaccaagctgaccgtcctaggtgacgtacgcgagcccaaatcttctgacaaaactcacacatgcccaccgtgcccagcacct
gaactcctgggtggaccgtcagtcttcctcttccccccaaaacccaaggacaccctcatgatctcccggacccctgaggtcacat
gcgtggtggtggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggactggctgaatg
gcaaggagtacaagtgcaaggtctccaacaaagccctcccagcccccatcgagaaaaccatctccaaagccaaagggcag
ccccgagaaccacaggtgtacaccctgcccccatcccgggatgagctgaccaagaaccaggtcagcctgacctgcctggtca
aaggcttctatccaagcgacatcgccgtggagtgggagagcaatgggcagccggagaacaactacaagaccacgcctcccg
tgctggactccgacggctccttcttcctctacagcaagctcaccgtggacaagagcaggtggcagcaggggaacgtottctcatg
ctccgtgatgcatgaggctctgcacaaccactacacgcagaagagcctctccctgtctccgggtaaatga (SEQ ID NO: 186)
>HER033_Protein_leader-stop
MEAPAQLLFLLLLWLPDTTGQVQLQQSGAEVKKPGSSVKVSCKASGGTISNYAISWVRLAP
GQGLEWMGSIVPLHGTTNFAQKFQGRVTITADESTSTSYMEVNVLTYEDTAMYYCASLNW
GYWGRGTLVTVSSGGGGSGGGGSGGGGSALNFMLTQPHSVSESPGKTVTISCTGSSGSI
ASNYVQWYQQRPDSAPTTVIYEDNRRSSGVPDRFSGSIDSSSNSASLSISGLKTEDEADYY
CQSYDSSGHVVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEAL
HNHYTQKSLSLSPGK SEQ ID NO: 187)
>HER034_CDS
atggaagcaccagcgcagcttctcttcctcctgctactctggctcccagataccaccggtgaggtgcagctggtgcagtctggggc
agaggtgaaaaagcccggggagtctctgaagatctcctgtaagggttttggatacaattttcgcagcgcctggatcggctgggtgc
gccagatgcccggcaaaggcctggagtggatgggggtcatctatcctggtgactctgatgtcagatacagtccgtccttccaagg
ccaggtcaccatctcagccgacaagtccatcagtaccgcctacctgcagtggagcagcctgaaagcctcggacaccgccatgt
attattgtacgagacccgtagggcagtgggtggactctgactattggggcaagggaaccctggtcaccgtctcgagtggaggcg
gcggttcaggcggaggtggctctggcggtggcggaagtgcacagtctgtgttgacgcagccgccctcagcgtctgggaccccc
ggacagagggtcaccatctcttgttctggaagcagctccaacatcggaactaatactgtgaactggtaccagcagcttccaggaa
cggcccccaaactcctcatctatactagtaatcagcggccctcaggggtccctgcccgcttctctgcctccaactctggcacctcag
cctccctggccatcagtgggctccggtccgaggatgaggctgattattattgtgcagcgtgggatgacaagttgagtggtgcggtgt
tcggcggagggaccaagctgaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcat
aatgccaagacaaagccgcgggaggagcagtacaacagcacgtaccgtgtggtcagcgtcctcaccgtcctgcaccaggac
tggctgaatggcaaggagtacaagtgcaaggtctccaacaaagccctcccagcccccatcgagaaaaccatctccaaagcca
aagggcagccccgagaaccacaggtgtacaccctgcccccatcccgggatgagctgaccaagaaccaggtcagcctgacct
gcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 188)
>HER034_Protein_leader-stop
MEAPAQLLFLLLLWLPDTTGEVQLVQSGAEVKKPGESLKISCKGFGYNFRSAWIGWVRQM
PGKGLEWMGVIYPGDSDVRYSPSFQGQVTISADKSISTAYLQWSSLKASDTAMYYCTRPV
GQWVDSDYWGKGTLVTVSSGGGGSGGGGSGGGGSAQSVLTQPPSASGTPGQRVTISCS
GSSSNIGTNTVNWYQQLPGTAPKLLIYTSNQRPSGVPARFSASNSGTSASLAISGLRSEDE
ADYYCAAWDDKLSGAVFGGGTKLTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 189)
>HER035_CDS
atggaagcaccagcgcagcttctcttcctcctgctactctggctcccagataccaccggtgaggtgcagctgttggagtctggggg
aggcttggtacagcctggggggtccctgagactctcctgtgcagcctctggattcacctttagcagctatgccatgagctgggtccg
ccaggctccagggaaggggctggagtgggtctcagctattagtggtagtggtggtagcacatactacgcagactccgtgaaggg
ccggttcaccatctccagagacaattccaagaacacgctgtatctgcaaatgaacagcctgagagccgaggacacggccgtgt
attactgtgcgagacagtcgggcgcggactggtacttcgatctctggggccgaggcaccctggtcaccgtctcgagtggaggcg
gcggttcaggcggaggtggctctggcggtggcggaagtgcacaggctgtgctgactcagccgtccgcagtttctggggccccag
ggcagagggtcaccatctcctgcactgggaccagctccaacatcgggacaaactatcttgtacactggtatcagcaacgtccag
gaacagccccccaactcctcgtctctggtaacaacactcgaccctctggggtcactgaccggttctctgtctccaagtctgccactt
cagcctccctggccatcactgggctccaggctgaggatgaggctgattattactgccagacctatgacatcaacttgagggtttgg
gtgttcggcggagggaccaaggtcaccgtcctaggtgacgtacgcgagcccaaatcttctgacaaaactcacacatgcccacc
gtgcccagcacctgaactcctgggtggaccgtcagtcttcctcttccccccaaaacccaaggacaccctcatgatctcccggacc
cctgaggtcacatgcgtggtggtggacgtgagccacgaagaccctgaggtcaagttcaactggtacgtggacggcgtggaggt
gcataatgccaagacaaagccgcgggaggagcagtacaacagcacgtaccgtgtggtcagcgtcctcaccgtcctgcacca
ggactggctgaatggcaaggagtacaagtgcaaggtctccaacaaagccctcccagcccccatcgagaaaaccatctccaa
agccaaagggcagccccgagaaccacaggtgtacaccctgcccccatcccgggatgagctgaccaagaaccaggtcagcc
tgacctgcctggtcaaaggcttctatccaagcgacatcgccgtggagtgggagagcaatgggcagccggagaacaactacaa
gaccacgcctcccgtgctggactccgacggctccttcttcctctacagcaagctcaccgtggacaagagcaggtggcagcagg
ggaacgtcttctcatgctccgtgatgcatgaggctctgcacaaccactacacgcagaagagcctctccctgtctccgggtaaatga (SEQ ID NO: 190)
>HER035_Protein_leader-stop
MEAPAQLLFLLLLWLPDTTGEVQLLESGGGLVQPGGSLRLSCAASGFTFSSYAMSWVRQA
PGKGLEWVSAISGSGGSTYYADSVKGRFTISRDNSKNTLYLQMNSLRAEDTAVYYCARQS
GADWYFDLWGRGTLVTVSSGGGGSGGGGSGGGGSAQAVLTQPSAVSGAPGQRVTISCT
GTSSNIGTNYLVHWYQQRPGTAPQLLVSGNNTRPSGVTDRFSVSKSATSASLAITGLQAED
EADYYCQTYDINLRVWVFGGGTKVTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 191)
>HER036_CDS
atggaagcaccagcgcagcttctcttcctcctgctactctggctcccagataccaccggtgaggtgcagctggtggagactgggg
gaggcgtggtccagcctggggggtccctgagcctctcctgtgcagcgtctggattcaccttcagtagctatggcatgcagtgggtc
cgccaggctccaggcaaggggctggagtgggtggcgtttatacggtacgatggaagtagtgaatactatgcagactccgtgaa
gggccgattcaccatctccagagacaattccaagaacacgctgtatctgcaaatgaacagcctgagagctgaggacacggctg
tgtattactgtggaagaacgctggagtctagtttgtggggcaagggaaccctggtcaccgtctcgagtggtggaggcggttcagg
cggaggtggcagcggcggtggcggatcgcagtctgtgttgacgcagccgccctcagtgtctgcggccccaggacagaaggtc
accatttcctgctctggaagcacctccaacattgggaataattatgtctcctggtaccaacagcacccaggcaaagcccccaaac
tcatgatttatgatgtcagtaagcggccctcaggggtccctgaccgattctctggctccaagtctggcaactcagcctccctggacat
cagtgggctccagtctgaggatgaggctgattattactgtgcagcatgggatgacagcctgagtgaatttctcttcggaactaggac
caagctgaccgtcctaggtgacgtacgcgagcccaaatcttctgacaaaactcacacatgcccaccgtgcccagcacctgaac
tcctgggtggaccgtcagtcttcctcttccccccaaaacccaaggacaccctcatgatctcccggacccctgaggtcacatgcgtg
gtggtggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatggcaa
ggagtacaagtgcaaggtctccaacaaagccctcccagcccccatcgagaaaaccatctccaaagccaaagggcagcccc
gagaaccacaggtgtacaccctgcccccatcccgggatgagctgaccaagaaccaggtcagcctgacctgcctggtcaaagg
cttctatccaagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgctccg
tgatgcatgaggctctgcacaaccactacacgcagaagagcctctccctgtctccgggtaaatga (SEQ ID NO: 192)
>HER036_Protein_leader-stop
MEAPAQLLFLLLLWLPDTTGEVQLVETGGGVVQPGGSLSLSCAASGFTFSSYGMQWVRQA
PGKGLEWVAFIRYDGSSEYYADSVKGRFTISRDNSKNTLYLQMNSLRAEDTAVYYCGRTLE
SSLWGKGTLVTVSSGGGGSGGGGSGGGGSQSVLTQPPSVSAAPGQKVTISCSGSTSNIG
NNYVSWYQQHPGKAPKLMIYDVSKRPSGVPDRFSGSKSGNSASLDISGLQSEDEADYYCA
AWDDSLSEFLFGTRTKLTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHN
HYTQKSLSLSPGK (SEQ ID NO: 193)
>HER037_CDS
atggaagcaccagcgcagcttctcttcctcctgctactctggctcccagataccaccggtcaggtgcagctggtgcagtctggagc
tgaggtgaagaagcctgggtcctcggtgaaggtctcctgcaaggcttctggttacacctttaccagctatggtatcagctgggtgcg
acaggcccctggacaagggcttgagtggatgggatggatcagcgcttacaatggtaacacaaactatgcacagaagctccag
ggcagagtcaccatgaccacagacacatccacgagcacagcctacatggagctgaggagcctgagatctgacgacacggc
cgtgtattactgtgcgagagtcccgggcgtaagtgggagctatccagactactactacatggacgtctggggcaagggaaccct
ggtcaccgtctcctcaggtggaggcggttcaggcggtggcagcggcggtggcggatcggacatccagatgacccagtctccttc
caccctgtctgcatctattggagacagagtcaccatcacctgccgggccagtgagggtatttatcactggttggcctggtatcagca
gaagccagggaaagctcctaaactcctgatctataaggcctctagtttagccagtggggccccatcaaggttcagcggcagtgg
atctgggacagatttcactctcaccatcagcagcctgcagcctgatgattttgcaacttattactgccaacaatatagtaattatccgc
tcactttcggcggagggaccaagctggagatcaaacgtgacgtacgcgagcccaaatcttctgacaaaactcacacatgccca
ccgtgcccagcacctgaactcctgggtggaccgtcagtcttcctcttccccccaaaacccaaggacaccctcatgatctcccgga
cccctgaggtcacatgcgtggtggtggacgtgagccacgaagaccctgaggtcaagttcaactggtacgtggacggcgtggag
gtgcataatgccaagacaaagccgcgggaggagcagtacaacagcacgtaccgtgtggtcagcgtcctcaccgtcctgcacc
aggactggctgaatggcaaggagtacaagtgcaaggtctccaacaaagccctcccagcccccatcgagaaaaccatctcca
aagccaaagggcagccccgagaaccacaggtgtacaccctgcccccatcccgggatgagctgaccaagaaccaggtcagc
ctgacctgcctggtcaaaggcttctatccaagcgacatcgccgtggagtgggagagcaatgggcagccggagaacaactaca
agaccacgcctcccgtgctggactccgacggctccttcttcctctacagcaagctcaccgtggacaagagcaggtggcagcag
gggaacgtcttctcatgctccgtgatgcatgaggctctgcacaaccactacacgcagaagagcctctccctgtctccggg
taaatga (SEQ ID NO: 194)
>HER037_Protein_leader-stop
MEAPAQLLFLLLLWLPDTTGQVQLVQSGAEVKKPGSSVKVSCKASGYTFTSYGISWVRQA
PGQGLEWMGWISAYNGNTNYAQKLQGRVTMTTDTSTSTAYMELRSLRSDDTAVYYCARV
PGVSGSYPDYYYMDVWGKGTLVTVSSGGGGSGGGSGGGGSDIQMTQSPSTLSASIGDRV
TITCRASEGIYHWLAWYQQKPGKAPKLLIYKASSLASGAPSRFSGSGSGTDFTLTISSLQPD
DFATYYCQQYSNYPLTFGGGTKLEIKRDVREPKSSDKTHTCPPCPAPELLGGPSVFLFPPK
PKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVM
HEALHNHYTQKSLSLSPGK (SEQ ID NO: 195)
>HER038_CDS
atggaagcaccagcgcagcttctcttcctcctgctactctggctcccagataccaccggtgaggtgcagctgttggagtctggggg
aggcttggtacagcctggggggtccctgagactctcctgtgcagcctctggattcacctttagcagctatgccatgagctgggtccg
ccaggctccagggaaggggctggagtgggtctcagctattagtggtagtggtggtagcacatactacgcagactccgtgaaggg
ccggttcaccatctccagagacaattccaagaacacgctgtatctgcaaatgaacagcctgagagccgaggacacggccgtgt
attactgtgcgagatggaggcctcttctagactaccactttgaccaatggggccaagggacaatggtcaccgtctcgagtggagg
cggcggttcaggcggaggtggctctggcggtggcggaagtgcacagtctgtgctgactcagccaccctcagcgtctgggaccc
ccggacagacggtaacaatctcttgttctggaagcagctccaacatcggaagtagtgttgttaattggtaccagcagttcccagga
acggcccccaaagtcctcgtctatagtaacactcagcggccctcaggggtccctgaccgattctctggctccaggtctggcacctc
agcctccctggccatcagtgggctccagtctgaggatgaggctgattattactgtttagcatgggatgccagcctgaatggttgggt
gttcggcggagggaccaagctgaccgtcctaggtgacgtacgcgagcccaaatcttctgacaaaactcacacatgcccaccgt
gcccagcacctgaactcctgggtggaccgtcagtcttcctcttccccccaaaacccaaggacaccctcatgatctcccggacccc
tgaggtcacatgcgtggtggtggacgtgagccacgaagaccctgaggtcaagttcaactggtacgtggacggcgtggaggtgc
ataatgccaagacaaagccgcgggaggagcagtacaacagcacgtaccgtgtggtcagcgtcctcaccgtcctgcaccagg
actggctgaatggcaaggagtacaagtgcaaggtctccaacaaagccctcccagcccccatcgagaaaaccatctccaaagc
caaagggcagccccgagaaccacaggtgtacaccctgcccccatcccgggatgagctgaccaagaaccaggtcagcctga
cctgcctggtcaaaggcttctatccaagcgacatcgccgtggagtgggagagcaatgggcagccggagaacaactacaagac
cacgcctcccgtgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtaaatga (SEQ ID NO: 196)
>HER038_Protein_leader-stop
MEAPAQLLFLLLLWLPDTTGEVQLLESGGGLVQPGGSLRLSCAASGFTFSSYAMSWVRQA
PGKGLEWVSAISGSGGSTYYADSVKGRFTISRDNSKNTLYLQMNSLRAEDTAVYYCARWR
PLLDYHFDQWGQGTMVTVSSGGGGSGGGGSGGGGSAQSVLTQPPSASGTPGQTVTISC
SGSSSNIGSSVVNWYQQFPGTAPKVLVYSNTQRPSGVPDRFSGSRSGTSASLAISGLQSE
DEADYYCLAWDASLNGWVFGGGTKLTVLGDVREPKSSDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSC
SVMHEALHNHYTQKSLSLSPGK (SEQ ID NO: 197)
>HER039_CDS
atggaagcaccagcgcagcttctcttcctcctgctactctggctcccagataccaccggtgaggtgcagctggtggagactgggg
gaggcttggtacagcctggggggtccctgagactctcctgtgcagcctctggattcaccttcagtagctatggcatgaactgggtcc
gccaggctccagggaaggggctggagtgggtttcatacattagtagttctggtaataccatattctacgcagactctgtgaagggc
cgattcaccatctccagagacagtgccaagaattcagtgtctctgcagatgaacagcctgagagacgaggacacggctgtgtat
tactgtgcttcctactactcctactactacggtatggacgcctggggccaggggacaatggtcaccgtctcgagtggaggcggcg
gttcaggcggaggtggctctggcggtggcggaagtgcactttcctatgtgctgactcagccaccctcagcgtctgggacccccgg
gcagagggtcaccatctcttgttctggaagcagctccaacatcggaagtaatactgtaaactggtaccagcagctcccaggaac
ggcccccaaactcctcatctatagtaataatcagcggccctcaggggtccctgaccgattctctggctccaagtctggcacctcag
cctccctggccatcagtgggctgcggtccgaggatgaggctgattattactgtgcagcatgggattacagcctgagtggttgggtgt
tcggcggagggaccaaggtcaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcat
aatgccaagacaaagccgcgggaggagcagtacaacagcacgtaccgtgtggtcagcgtcctcaccgtcctgcaccaggac
tggctgaatggcaaggagtacaagtgcaaggtctccaacaaagccctcccagcccccatcgagaaaaccatctccaaagcca
aagggcagccccgagaaccacaggtgtacaccctgcccccatcccgggatgagctgaccaagaaccaggtcagcctgacct
gcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 198)
>HER039_Protein_leader-stop
MEAPAQLLFLLLLWLPDTTGEVQLVETGGGLVQPGGSLRLSCAASGFTFSSYGMNWVRQA
PGKGLEWVSYISSSGNTIFYADSVKGRFTISRDSAKNSVSLQMNSLRDEDTAVYYCASYYS
YYYGMDAWGQGTMVTVSSGGGGSGGGGSGGGGSALSYVLTQPPSASGTPGQRVTISCS
GSSSNIGSNTVNWYQQLPGTAPKLLIYSNNQRPSGVPDRFSGSKSGTSASLAISGLRSEDE
ADYYCAAWDYSLSGWVFGGGTKVTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 199)
>HER071_CDS
atggaagcaccagcgcagcttctcttcctcctgctactctggctcccagataccaccggtgaggtgcagctggtggagactgggg
aaggcctggtcaagcctggggggtccctgagactctcctgtacagcctctggattcaccttcaggagttatagcttgaactgggtcc
gccaggctccagggcaggggctggagtgggtctcatccattagtagtactagtacttacatatactacgcagactcggtgaaggg
ccgattcaccatctccagagacgacgccaagaacacactgtatctgcaaatgaacagcctgagagccgaagacacagctgc
atattactgtgttagactgggatctggtgggggatattttcctgactactggggcaggggcaccctggtcaccgtctcgagtggtgga
ggcggttcaggcggaggtggcagcggcggtggcggatcgtctgagctgactcaggaccctgctgtgtctgtggccttgggacag
acagtcaggatcacatgccaaggagacagcctcagaagctattatgcaagctggtaccagcagaagccaggacaggcccct
gtacttgtcatctatggtaaaaacaaccggccctcagggatcccagaccgattctctggctccagctcaggaaacacagcttcctt
gaccatcactggggctcaggcggaagatgaggctgactattactgtaactcccgggacagcagtggtaaccatgtggtattcgg
cggagggaccaagctgaccgtcctaggtgacgtacgcgagcccaaatcttctgacaaaactcacacatgcccaccgtgccca
gcacctgaactcctgggtggaccgtcagtcttcctcttccccccaaaacccaaggacaccctcatgatctcccggacccctgagg
tcacatgcgtggtggtggacgtgagccacgaagaccctgaggtcaagttcaactggtacgtggacggcgtggaggtgcataatg
ccaagacaaagccgcgggaggagcagtacaacagcacgtaccgtgtggtcagcgtcctcaccgtcctgcaccaggactggct
gaatggcaaggagtacaagtgcaaggtctccaacaaagccctcccagcccccatcgagaaaaccatctccaaagccaaag
ggcagccccgagaaccacaggtgtacaccctgcccccatcccgggatgagctgaccaagaaccaggtcagcctgacctgcct
ggtcaaaggcttctatccaagcgacatcgccgtggagtgggagagcaatgggcagccggagaacaactacaagaccacgcc
tcccgtgctggactccgacggctccttcttcctctacagcaagctcaccgtggacaagagcaggtggcagcaggggaacgtcttc
tcatgctccgtgatgcatgaggctctgcacaaccactacacgcagaagagcctctccctgtctccgggtaaatga (SEQ ID NO: 200)
>HER071_Protein_leader-stop
MEAPAQLLFLLLLWLPDTTGEVQLVETGEGLVKPGGSLRLSCTASGFTFRSYSLNWVRQAP
GQGLEWVSSISSTSTYIYYADSVKGRFTISRDDAKNTLYLQMNSLRAEDTAAYYCVRLGSG
GGYFPDYWGRGTLVTVSSGGGGSGGGGSGGGGSSELTQDPAVSVALGQTVRITCQGDS
LRSYYASWYQQKPGQAPVLVIYGKNNRPSGIPDRFSGSSSGNTASLTITGAQAEDEADYYC
NSRDSSGNHVVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEAL
HNHYTQKSLSLSPGK (SEQ ID NO: 201)
>HER072_CDS
atggaagcaccagcgcagcttctcttcctcctgctactctggctcccagataccaccggtcaggtgcagctggtggagtctggggc
tgaggtgaagaagcctggggcctcagtgaaggtctcctgcaaggcttctggatacaccttcaccagttatgatatcaactgggtgc
gacaggcccccggacaaaggcttgagtggatgggatggatcaacgctggcaatggtaacacaaaatattcacagaagttcca
gggcagagtcaccattaccagggacacatccgcgagcacagcctacatggagctgaggagcctgagatctgacgacacggc
cgtgtattactgtgcgagagggaggagctatggccacccgtactactttgactactggggccagggaaccctggtcaccgtctcg
agtggtggaggcggttcaggcggaggtggcagcggcggtggcggatcgcagtctgtgctgactcagcctgcctccgtgtctggg
tctcctggacagtcgatcaccatctcctgcactggaaccagcagtgacgttggtggttataactatgtctcctggtaccaacaacac
ccaggcaaagcccccaaactcatgatttatgagggcagtaagcggccctcaggggtttctaatcgcttctctggctccaagtctgg
caacacggcctccctgacaatctctgggctccaggctgaggacgaggctgattattactgcagctcatatacaaccaggagcac
tcgagttttcggcggagggaccaagctgaccgtcctaggtgacgtacgcgagcccaaatcttctgacaaaactcacacatgccc
accgtgcccagcacctgaactcctgggtggaccgtcagtcttcctcttccccccaaaacccaaggacaccctcatgatctcccgg
acccctgaggtcacatgcgtggtggtggacgtgagccacgaagaccctgaggtcaagttcaactggtacgtggacggcgtgga
ggtgcataatgccaagacaaagccgcgggaggagcagtacaacagcacgtaccgtgtggtcagcgtcctcaccgtcctgcac
caggactggctgaatggcaaggagtacaagtgcaaggtctccaacaaagccctcccagcccccatcgagaaaaccatctcca
aagccaaagggcagccccgagaaccacaggtgtacaccctgcccccatcccgggatgagctgaccaagaaccaggtcagc
ctgacctgcctggtcaaaggcttctatccaagcgacatcgccgtggagtgggagagcaatgggcagccggagaacaactaca
agaccacgcctcccgtgctggactccgacggctccttcttcctctacagcaagctcaccgtggacaagagcaggtggcagcag
gggaacgtcttctcatgctccgtgatgcatgaggctctgcacaaccactacacgcagaagagcctctccctgtctccggg
taaatga (SEQ ID NO: 202)
>HER072_Protein_leader-stop
MEAPAQLLFLLLLWLPDTTGQVQLVESGAEVKKPGASVKVSCKASGYTFTSYDINWVRQAP
GQRLEWMGWINAGNGNTKYSQKFQGRVTITRDTSASTAYMELRSLRSDDTAVYYCARGR
SYGHPYYFDYWGQGTLVTVSSGGGGSGGGGSGGGGSQSVLTQPASVSGSPGQSITISCT
GTSSDVGGYNYVSWYQQHPGKAPKLMIYEGSKRPSGVSNRFSGSKSGNTASLTISGLQAE
DEADYYCSSYTTRSTRVFGGGTKLTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 203)
>HER073_CDS
atggaagcaccagcgcagcttctcttcctcctgctactctggctcccagataccaccggtaaggtgcagctggtgcagtctgggac
agaggtgaaaaagcccggggagtctctgaagatctcctgtcagggttctggatacaggtttagtagtgactggattgcctgggtgc
gccagatgcccgggaaaggcctggagtggatggggattgtctatcctggtgactctgataccagatatagcccgtccttccaagg
ccaagtcaccatctcagccgacaagtccatcagtactgcctacctgcagtggagcggcctgaaggcctcggacaccgccaagt
attactgtgcgagagtgcaacaggcagtgggagctaaaggttatgctatggacgtctggggcaagggaaccctggtcaccgtct
cgagtggaggcggcggttcaggcggaggtggctctggcggtggcggaagtgcacagactgtggtgatccaggagccatcgttc
tcagtgtcccctggagggacagtcacactcacttgtggcttgagctctggctcagtctctaccagttactaccccagctggtaccgg
cagaccccaggccaggctccacacacactcattcacaacacaaagattcgctcctctggggtccctgatcgcttctctggctccat
ccttgggaacaatgctgccctcaccatcacgggggcccaggcagatgatgaatctgattattactgtcttttgtatatgggtagcgg
catttacgtgttcggcggagggaccaagctgaccgtcctaggtgacgtacgcgagcccaaatcttctgacaaaactcacacatg
cccaccgtgcccagcacctgaactcctgggtggaccgtcagtcttcctcttccccccaaaacccaaggacaccctcatgatctcc
cggacccctgaggtcacatgcgtggtggtggacgtgagccacgaagaccctgaggtcaagttcaactggtacgtggacggcgt
ggaggtgcataatgccaagacaaagccgcgggaggagcagtacaacagcacgtaccgtgtggtcagcgtcctcaccgtcctg
caccaggactggctgaatggcaaggagtacaagtgcaaggtctccaacaaagccctcccagcccccatcgagaaaaccatct
ccaaagccaaagggcagccccgagaaccacaggtgtacaccctgcccccatcccgggatgagctgaccaagaaccaggtc
agcctgacctgcctggtcaaaggcttctatccaagcgacatcgccgtggagtgggagagcaatgggcagccggagaacaact
acaagaccacgcctcccgtgctggactccgacggctccttcttcctctacagcaagctcaccgtggacaagagcaggtggcag
caggggaacgtcttctcatgctccgtgatgcatgaggctctgcacaaccactacacgcagaagagcctctccctgtctccgggta
aatga (SEQ ID NO: 204)
>HER073_Protein_leader-stop
MEAPAQLLFLLLLWLPDTTGKVQLVQSGTEVKKPGESLKISCQGSGYRFSSDWIAWVRQM
PGKGLEWMGIVYPGDSDTRYSPSFQGQVTISADKSISTAYLQWSGLKASDTAKYYCARVQ
QAVGAKGYAMDVWGKGTLVTVSSGGGGSGGGGSGGGGSAQTVVIQEPSFSVSPGGTVT
LTCGLSSGSVSTSYYPSWYRQTPGQAPHTLIHNTKIRSSGVPDRFSGSILGNNAALTITGAQ
ADDESDYYCLLYMGSGIYVFGGGTKLTVLGDVREPKSSDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSC
SVMHEALHNHYTQKSLSLSPGK (SEQ ID NO: 205)
>HER074_CDS
atggaagcaccagcgcagcttctcttcctcctgctactctggctcccagataccaccggtgaagtgcagctggtgcagtctggggc
tgaggtgaagaagcctggggcctcagtgagggtctcctgcaagggttctggaaacaccttcaccggccactacatccactgggt
gcgacaggcccctggacaaggacttgagtggctgggatggatcgaccctaacactggtgacatacagtattcagaaaactttaa
gggctcggtcaccttgaccagggacccatccatcaactcagtcttcatggacctgatcaggctgacatctgacgacacggccatg
tattactgtgcgagagaaggtgccgggctcgccaactactattactacggtctggacgtctggggccgagggacaatggtcacc
gtctcgagtggaggcggcggttcaggcggaggtggctctggcggtggcggaagtgcacagactgtggtgctccaggagccttc
gttctcagtgtcccctggggggacagtcacactcacttgtggcttgaactttggctcagtctctactgcttactaccccagttggtacca
gcagaccccaggccaagctccacgcacgctcatctacggcacaaatattcgttcctctggggtcccggatcgcttctctggctcca
tcgtagggaacaaagctgccctcaccatcacgggggcccagacagaagatgagtctgattattattgtgcgctgtatatgggtagt
ggcatgctcttcggcggcgggaccaaggtcaccgtcctaggtgacgtacgcgagcccaaatcttctgacaaaactcacacatgc
ccaccgtgcccagcacctgaactcctgggtggaccgtcagtcttcctcttccccccaaaacccaaggacaccctcatgatctccc
ggacccctgaggtcacatgcgtggtggtggacgtgagccacgaagaccctgaggtcaagttcaactggtacgtggacggcgtg
gaggtgcataatgccaagacaaagccgcgggaggagcagtacaacagcacgtaccgtgtggtcagcgtcctcaccgtcctgc
accaggactggctgaatggcaaggagtacaagtgcaaggtctccaacaaagccctcccagcccccatcgagaaaaccatctc
caaagccaaagggcagccccgagaaccacaggtgtacaccctgcccccatcccgggatgagctgaccaagaaccaggtca
gcctgacctgcctggtcaaaggcttctatccaagcgacatcgccgtggagtgggagagcaatgggcagccggagaacaacta
caagaccacgcctcccgtgctggactccgacggctccttcttcctctacagcaagctcaccgtggacaagagcaggtggcagc
aggggaacgtcttctcatgctccgtgatgcatgaggctctgcacaaccactacacgcagaagagcctctccctgtctccgggtaa
atga (SEQ ID NO: 206)
>HER074_Protein_leader-stop
MEAPAQLLFLLLLWLPDTTGEVQLVQSGAEVKKPGASVRVSCKGSGNTFTGHYIHWVRQA
PGQGLEWLGWIDPNTGDIQYSENFKGSVTLTRDPSINSVFMDLIRLTSDDTAMYYCAREGA
GLANYYYYGLDVWGRGTMVTVSSGGGGSGGGGSGGGGSAQTVVLQEPSFSVSPGGTVT
LTCGLNFGSVSTAYYPSWYQQTPGQAPRTLIYGTNIRSSGVPDRFSGSIVGNKAALTITGAQ
TEDESDYYCALYMGSGMLFGGGTKVTVLGDVREPKSSDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSC
SVMHEALHNHYTQKSLSLSPGK (SEQ ID NO: 207)
>HER075_CDS
atggaagcaccagcgcagcttctcttcctcctgctactctggctcccagataccaccggtgaagtgcagctggtgcagtctggggc
tgaagtgaagaagcctggggcctcagtgaaggtctcttgtcaggcttctggatacaccttcagcgggcactatatgcacttggtgc
gacaggcccctggacaagggcttgagtggatggggtggatccaccctaccagtggtggcacaacctatgcacagaagtttcag
ggccgggtcgttatgaccagggacacgtccatcagcacagcctacatggaactgagtaggctgacatctgacgacacggccgt
gtattactgtgcaagaatgtcccaaaactatgatgcttttgatatctggggccaagggacaatggtcaccgtctcgagtggaggcg
gcggttcaggcggaggtggctctggcggtggcggaagtgcacaggctgtgctgactcagccgtcctcagtgtctggggccccag
ggcagagggtcaccatctcctgcactgggagcagctccaacatcggggcaggttatgatgtaaactggtaccaacaatttccag
gaacagcccccaaaattatcgtctatggcgatcggccctcaggggcccctgaccgattctctggctccaagtctggcacctcagc
ctccctggcaatcactggactccgggctgaggatgaggctgattattactgccagtcctgggacagtcgcctgagtagttatgtcttc
ggaactgggaccaaggtcaccgtcctaggtgacgtacgcgagcccaaatcttctgacaaaactcacacatgcccaccgtgccc
agcacctgaactcctgggtggaccgtcagtcttcctcttccccccaaaacccaaggacaccctcatgatctcccggacccctgag
gtcacatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggatgagctgaccaagaaccaggtcagcctgacctgc
ctggtcaaaggcttctatccaagcgacatcgccgtggagtgggagagcaatgggcagccggagaacaactacaagaccacg
cctcccgtgctggactccgacggctccttcttcctctacagcaagctcaccgtggacaagagcaggtggcagcaggggaacgtc
ttctcatgctccgtgatgcatgaggctctgcacaaccactacacgcagaagagcctctccctgtctccgggtaaatga (SEQ ID NO: 208)
>HER075_Protein_leader-stop
MEAPAQLLFLLLLWLPDTTGEVQLVQSGAEVKKPGASVKVSCQASGYTFSGHYMHLVRQA
PGQGLEWMGWIHPTSGGTTYAQKFQGRVVMTRDTSISTAYMELSRLTSDDTAVYYCARM
SQNYDAFDIWGQGTMVTVSSGGGGSGGGGSGGGGSAQAVLTQPSSVSGAPGQRVTISC
TGSSSNIGAGYDVNWYQQFPGTAPKIIVYGDRPSGAPDRFSGSKSGTSASLAITGLRAEDE
ADYYCQSWDSRLSSYVFGTGTKVTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 209)
>HER076_CDS
atggaagcaccagcgcagcttctcttcctcctgctactctggctcccagataccaccggtgaggtgcagctggtgcagtctggggc
agaggtgaaaaagcccggagagtctctgaagatctcctgtaagggctctggatacacctttaccaaccactggatcgcctgggt
gcgccagatgcccgggaaaggcctggagtggatgggcatcatctatcctggtgactctgaaacgaggtacagcccgtccttcca
aggccacgtcaccatctcagccgacaagtccatcagtaccgcctatttgcagtggagcaccctgaaggactcggactccgccat
gtacttctgtgtgagacaggcccgtggctgggacgacggacgggctggatattattattccggtatggacgcctggggccaggga
accctggtcaccgtctcgagtggaggcggcggttcaggcggaggtggctctggcggtggcggaagtgcacaggctgtggtgct
ccaggagccatcgttctcagtgtcccctggagggacagtcacactcacctgtggcttgcgctctgggtcagtctctactagtcacta
ccccagctggtaccagcagaccccaggccaggctccacgcacgctcatttacagcacaaacactcgctcttctggggtccctga
tcgcttctctggctccatccttgggaacaaagctgccctcaccatcacgggggcccaggcagatgatgaatctaattattactgtat
gctatacatgggcagtggcatgtatgtgttcggcggagggaccaaggtcaccgtcctaggtgacgtacgcgagcccaaatcttct
gacaaaactcacacatgcccaccgtgcccagcacctgaactcctgggtggaccgtcagtcttcctcttccccccaaaacccaag
gacaccctcatgatctcccggacccctgaggtcacatgcgtggtggtggacgtgagccacgaagaccctgaggtcaagttcaa
ctggtacgtggacggcgtggaggtgcataatgccaagacaaagccgcgggaggagcagtacaacagcacgtaccgtgtggt
cagcgtcctcaccgtcctgcaccaggactggctgaatggcaaggagtacaagtgcaaggtctccaacaaagccctcccagcc
cccatcgagaaaaccatctccaaagccaaagggcagccccgagaaccacaggtgtacaccctgcccccatcccgggatga
gctgaccaagaaccaggtcagcctgacctgcctggtcaaaggcttctatccaagcgacatcgccgtggagtgggagagcaatg
ggcagccggagaacaactacaagaccacgcctcccgtgctggactccgacggctccttcttcctctacagcaagctcaccgtgg
acaagagcaggtggcagcaggggaacgtcttctcatgctccgtgatgcatgaggctctgcacaaccactacacgcagaagag
cctctccctgtctccgggtaaatga (SEQ ID NO: 210)
>HER076_Protein_leader-stop
MEAPAQLLFLLLLWLPDTTGEVQLVQSGAEVKKPGESLKISCKGSGYTFTNHWIAWVRQMP
GKGLEWMGIIYPGDSETRYSPSFQGHVTISADKSISTAYLQWSTLKDSDSAMYFCVRQARG
WDDGRAGYYYSGMDAWGQGTLVTVSSGGGGSGGGGSGGGGSAQAVVLQEPSFSVSPG
GTVTLTCGLRSGSVSTSHYPSWYQQTPGQAPRTLIYSTNTRSSGVPDRFSGSILGNKAALTI
TGAQADDESNYYCMLYMGSGMYVFGGGTKVTVLGDVREPKSSDKTHTCPPCPAPELLGG
PSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPGK (SEQ ID NO: 211)
>HER077_CDS
atggaagcaccagcgcagcttctcttcctcctgctactctggctcccagataccaccggtgaggtgcagctgttggagtctggggg
aggcttggtacagcctggggggtccctgagactctcctgtgcagcctctggattcacctttagcagctatgccatgagctgggtccg
ccaggctccagggaaggggctggagtgggtctcagctattagtggtagtggtggtagcacatactacgcagactccgtgaaggg
ccggttcaccatctccagagacaattccaagaacacgctgtatctgcaaatgaacagcctgagagccgaggacacggccgtgt
attactgtgcgagagatctgggaatagaccccctttggagtggttattacacaccccttgactattggggccgagggacaatggtc
accgtctcgagtggaggcggcggttcaggcggaggtggctctggcggtggcggaagtgcacacgttatactgactcaaccgcc
ctcagcgtctgggacccccgggcagagggtcaccatctcttgttctggaagcagctccaacatcggaagtaattccgttagctggt
accagcagctcccaggaacggcccccaaactcctcatgtatactaacaatcagcggccctcaggggtccctgaccgattctctg
gctccaagtctggcacctcagcctccctggccatcagtgggctccagtctgaggatgaggctgattattactgtgcgacatgggat
gccagcctgaatacttgggtgttcggcggagggaccaaggtcaccgtcctaggtgacgtacgcgagcccaaatcttctgacaaa
actcacacatgcccaccgtgcccagcacctgaactcctgggtggaccgtcagtcttcctcttccccccaaaacccaaggacacc
ctcatgatctcccggacccctgaggtcacatgcgtggtggtggacgtgagccacgaagaccctgaggtcaagttcaactggtac
gtggacggcgtggaggtgcataatgccaagacaaagccgcgggaggagcagtacaacagcacgtaccgtgtggtcagcgtc
ctcaccgtcctgcaccaggactggctgaatggcaaggagtacaagtgcaaggtctccaacaaagccctcccagcccccatcg
agaaaaccatctccaaagccaaagggcagccccgagaaccacaggtgtacaccctgcccccatcccgggatgagctgacc
aagaaccaggtcagcctgacctgcctggtcaaaggcttctatccaagcgacatcgccgtggagtgggagagcaatgggcagc
cggagaacaactacaagaccacgcctcccgtgctggactccgacggctccttcttcctctacagcaagctcaccgtggacaag
agcaggtggcagcaggggaacgtcttctcatgctccgtgatgcatgaggctctgcacaaccactacacgcagaagagcctctc
cctgtctccgggtaaatga SEQ ID NO: 212)
>HER077_Protein_leader-stop
MEAPAQLLFLLLLWLPDTTGEVQLLESGGGLVQPGGSLRLSCAASGFTFSSYAMSWVRQA
PGKGLEWVSAISGSGGSTYYADSVKGRFTISRDNSKNTLYLQMNSLRAEDTAVYYCARDL
GIDPLWSGYYTPLDYWGRGTMVTVSSGGGGSGGGGSGGGGSAHVILTQPPSASGTPGQ
RVTISCSGSSSNIGSNSVSWYQQLPGTAPKLLMYTNNQRPSGVPDRFSGSKSGTSASLAIS
GLQSEDEADYYCATWDASLNTWVFGGGTKVTVLGDVREPKSSDKTHTCPPCPAPELLGG
PSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPGK (SEQ ID NO: 213)
>HER078_CDS
atggaagcaccagcgcagcttctcttcctcctgctactctggctcccagataccaccggtgaggtccagctggtgcagtctggagc
tgaggtgaaggagcctggggcctcagtgaaggtctcctgcaaggcctctggttacgacttttccaactatggtttcagctgggtgcg
ccaggcccctggacaaggtcttgagtggatgggatggatcagctcttataatggttacacaaactatgcacagagactccaggg
cagagtcaccatgaccacagacacatccacgagcacagcctacatggagctgaggagcctgagatctgacgacacagctgt
ctattactgtgcgagagatcgaggacttggaaactggtacttcgatctctggggccaaggcaccctggtcaccgtctcgagtggtg
gaggcggttcaggcggaggtggcagcggcggtggcggatcgcagtctgtgctgactcagcctgcctccgtgtctgggtctcctgg
acagtcgatcaccatctcctgcactggaaccagcagtgacgttggtggttataactatgtctcctggtaccaacaacacccaggc
aaagcccccaaactcatgatttatgagggcagtaagcggccctcaggggtttctaatcgcttctctggctccaagtctggcaacac
ggcctccctgacaatctctgggctccaggctgaggacgaggctgattattactgcagctcatatacaaccaggagcactcgagttt
tcggcggagggaccaagctgaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcat
aatgccaagacaaagccgcgggaggagcagtacaacagcacgtaccgtgtggtcagcgtcctcaccgtcctgcaccaggac
tggctgaatggcaaggagtacaagtgcaaggtctccaacaaagccctcccagcccccatcgagaaaaccatctccaaagcca
aagggcagccccgagaaccacaggtgtacaccctgcccccatcccgggatgagctgaccaagaaccaggtcagcctgacct
gcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 214)
>HER078_Protein_leader-stop
MEAPAQLLFLLLLWLPDTTGEVQLVQSGAEVKEPGASVKVSCKASGYDFSNYGFSWVRQA
PGQGLEWMGWISSYNGYTNYAQRLQGRVTMTTDTSTSTAYMELRSLRSDDTAVYYCARD
RGLGNWYFDLWGQGTLVTVSSGGGGSGGGGSGGGGSQSVLTQPASVSGSPGQSITISCT
GTSSDVGGYNYVSWYQQHPGKAPKLMIYEGSKRPSGVSNRFSGSKSGNTASLTISGLQAE
DEADYYCSSYTTRSTRVFGGGTKLTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 215)
>HER079_CDS
atggaagcaccagcgcagcttctcttcctcctgctactctggctcccagataccaccggtcagatgcagctggtgcagtctggggg
aggcgtggtccagcctgggaggtccctgagactctcctgtgcagcctctggattcaccttcagtagctatggcatgcactgggtcc
gccaggctccaggcaaggggctggagtgggtggcagttatatcatatgatggaagtattaaatactatgcagactccgtgaagg
gccgattcaccatctccagagacaattccaagaacacactgtatctacaaatgaacagcctgagagccgaggacacgggcgtt
tattactgttcgaaagatcgctatagcagtggctggtacagctccgatgcttttgatatttggggccgagggacaatggtcaccgtct
cgagtggtggaggcggttcaggcggaggtggcagcggcggtggcggatcgtctgagctgactcaggaccctgctgtgtctgtgg
ccttgggacagacagtcaggatcacatgccaaggagacagcctcagaagctattatgcaagctggtaccagcagaagccag
gacaggcccctgtacttgtcatctatggtaaaaacaaccggccctcagggatcccagaccgattctctggctccagctcaggaaa
cacagcttccttgaccatcactggggctcaggcggaagatgaggctgactattactgtcattcccgggacagcagtggtaaccat
gtgcttttcggcggagggaccaagctgaccgtcctaggtgacgtacgcgagcccaaatcttctgacaaaactcacacatgccca
ccgtgcccagcacctgaactcctgggtggaccgtcagtcttcctcttccccccaaaacccaaggacaccctcatgatctcccgga
cccctgaggtcacatgcgtggtggtggacgtgagccacgaagaccctgaggtcaagttcaactggtacgtggacggcgtggag
gtgcataatgccaagacaaagccgcgggaggagcagtacaacagcacgtaccgtgtggtcagcgtcctcaccgtcctgcacc
aggactggctgaatggcaaggagtacaagtgcaaggtctccaacaaagccctcccagcccccatcgagaaaaccatctcca
aagccaaagggcagccccgagaaccacaggtgtacaccctgcccccatcccgggatgagctgaccaagaaccaggtcagc
ctgacctgcctggtcaaaggcttctatccaagcgacatcgccgtggagtgggagagcaatgggcagccggagaacaactaca
agaccacgcctcccgtgctggactccgacggctccttcttcctctacagcaagctcaccgtggacaagagcaggtggcagcag
gggaacgtcttctcatgctccgtgatgcatgaggctctgcacaaccactacacgcagaagagcctct
ccctgtctccgggtaaatga (SEQ ID NO: 216)
>HER079_Protein_leader-stop
MEAPAQLLFLLLLWLPDTTGQMQLVQSGGGVVQPGRSLRLSCAASGFTFSSYGMHWVRQ
APGKGLEWVAVISYDGSIKYYADSVKGRFTISRDNSKNTLYLQMNSLRAEDTGVYYCSKDR
YSSGWYSSDAFDIWGRGTMVTVSSGGGGSGGGGSGGGGSSELTQDPAVSVALGQTVRIT
CQGDSLRSYYASWYQQKPGQAPVLVIYGKNNRPSGIPDRFSGSSSGNTASLTITGAQAED
EADYYCHSRDSSGNHVLFGGGTKLTVLGDVREPKSSDKTHTCPPCPAPELLGGPSVFLFP
PKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCS
VMHEALHNHYTQKSLSLSPGK (SEQ ID NO: 217)
>HER080_CDS
atggaagcaccagcgcagcttctcttcctcctgctactctggctcccagataccaccggtgaggtgcagctggtgcagtctggggg
aggcgtggtccagcctgggaggtccctgagactctcctgtgcagcctctggattcaccttcagtagctatggcatgcactgggtcc
gccaggctccaggcaaggggctggagtgggtggcagttatatcatatgatggaagtattaaatactatgcagactccgtgaagg
gccgattcaccatctccagagacaattccaagaacacgctgtatctgcaaatgaacagcctgagagctgaggacacggctgtgt
attactgtgcgcgaactggtgaatatagtggctacgatacgagtggttacagcaattggggccaaggcaccctggtcaccgtctc
gagtggtggaggcggttcaggcggaggtggcagcggcggtggcggatcgcagtctgtgctgactcagccaccctcagcgtctg
ggacccccgggcagagggtcaccatctcttgttctggaagcagctccaacatcgggagtaacactgtaaactggtaccagcga
ctcccaggagcggccccccaactcctcatctacaataatgaccagcggccctcagggatccctgaccgattctctggctccaagt
ctggcacctcaggctccctggtcatcagtgggctccagtctgaagatgaggctgattactactgtgcgtcatgggatgacagtctga
atggtcgggtgttcggcggagggaccaagctgaccgtcctaggtgacgtacgcgagcccaaatcttctgacaaaactcacacat
gcccaccgtgcccagcacctgaactcctgggtggaccgtcagtcttcctcttccccccaaaacccaaggacaccctcatgatctc
ccggacccctgaggtcacatgcgtggtggtggacgtgagccacgaagaccctgaggtcaagttcaactggtacgtggacggcg
tggaggtgcataatgccaagacaaagccgcgggaggagcagtacaacagcacgtaccgtgtggtcagcgtcctcaccgtcct
gcaccaggactggctgaatggcaaggagtacaagtgcaaggtctccaacaaagccctcccagcccccatcgagaaaaccat
ctccaaagccaaagggcagccccgagaaccacaggtgtacaccctgcccccatcccgggatgagctgaccaagaaccagg
tcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggca
gcaggggaacgtcttctcatgctccgtgatgcatgaggctctgcacaaccactacacgcagaagagcctctccctgtctccgggt
aaatga (SEQ ID NO: 218)
>HER080_Protein_leader-stop
MEAPAQLLFLLLLWLPDTTGEVQLVQSGGGVVQPGRSLRLSCAASGFTFSSYGMHWVRQ
APGKGLEWVAVISYDGSIKYYADSVKGRFTISRDNSKNTLYLQMNSLRAEDTAVYYCARTG
EYSGYDTSGYSNWGQGTLVTVSSGGGGSGGGGSGGGGSQSVLTQPPSASGTPGQRVTI
SCSGSSSNIGSNTVNWYQRLPGAAPQLLIYNNDQRPSGIPDRFSGSKSGTSGSLVISGLQS
EDEADYYCASWDDSLNGRVFGGGTKLTVLGDVREPKSSDKTHTCPPCPAPELLGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVF
SCSVMHEALHNHYTQKSLSLSPGK (SEQ ID NO: 219)
>HER081_CDS
atggaagcaccagcgcagcttctcttcctcctgctactctggctcccagataccaccggtcaggtgcagctggtgcagtctggggg
aggcttggtccagccgggggggtccctgagactctcctgtgcagcctctggattcacgtttagtacctatgccatgagttgggcccg
ccaggctccagggaaggggctggagtgggtctcaagtattagtggtgatggtggaagaattctcgatgcagactccgcgaagg
gccggttcaccatctccagagacaattccaagaacacgctgtatctgcaaatgaacggcctgagagtcgaggacacggcccttt
attactgtgcgagagcggacggtaactactggggcagggggacaatggtcaccgtctcttcaggtggaggcggttcaggcgga
ggtggcagcggcggtggcggatcgcagtctgtgctgactcagcctgcctccgtgtctgggtctcctggacagtcgatcaccatctc
ctgcactggaaccagcagtgacgttggtggttataactatgtctcctggtaccaacaacacccaggcaaagcccccaaactcat
gatttatgagggcagtaagcggccctcaggggtttctaatcgcttctctggctccaagtctggcaacacggcctccctgacaatctc
tgggctccaggctgaggacgaggctgattattactgcagctcatatacaaccaggagcactcgagttttcggcggagggaccaa
gctgaccgtcctaggtgacgtacgcgagcccaaatcttctgacaaaactcacacatgcccaccgtgcccagcacctgaactcct
gggtggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaagc
cgcgggaggagcagtacaacagcacgtaccgtgtggtcagcgtcctcaccgtcctgcaccaggactggctgaatggcaagga
gtacaagtgcaaggtctccaacaaagccctcccagcccccatcgagaaaaccatctccaaagccaaagggcagccccgag
aaccacaggtgtacaccctgcccccatcccgggatgagctgaccaagaaccaggtcagcctgacctgcctggtcaaaggcttc
tatccaagcgacatcgccgtggagtgggagagcaatgggcagccggagaacaactacaagaccacgcctcccgtgctggac
tccgacggctccttcttcctctacagcaagctcaccgtggacaagagcaggtggcagcaggggaacgtcttctcatgctccgtgat
gcatgaggctctgcacaaccactacacgcagaagagcctctccctgtctccgggtaaatga (SEQ ID NO: 220)
>HER081_Protein_leader-stop
MEAPAQLLFLLLLWLPDTTGQVQLVQSGGGLVQPGGSLRLSCAASGFTFSTYAMSWARQA
PGKGLEWVSSISGDGGRILDADSAKGRFTISRDNSKNTLYLQMNGLRVEDTALYYCARADG
NYWGRGTMVTVSSGGGGSGGGGSGGGGSQSVLTQPASVSGSPGQSITISCTGTSSDVG
GYNYVSWYQQHPGKAPKLMIYEGSKRPSGVSNRFSGSKSGNTASLTISGLQAEDEADYYC
SSYTTRSTRVFGGGTKLTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHN
HYTQKSLSLSPGK (SEQ ID NO: 221)
>HER082_CDS
atggaagcaccagcgcagcttctcttcctcctgctactctggctcccagataccaccggtcaggtgcagctgcaggagtcggggg
gaggcgtggtccagcctggggggtccctgagactctcctgtgcagcgtctggattcaccttcagtggctatggcatgcactgggtc
cgccaggctccaggcaaggggctggagtgggtggcatctgtacggaacgatggaagtaatacatactacacagactccgtga
aggaccgattcaccatctccagagacaacaccaagaacacgctgtatctgcaaatgaacagcctgagagccgaggacacgg
ccgtatattactgtgccaagtcgagaagagtgatgtatggcacctcctattactttgactactggggcagaggcaccctggtcaccg
tctcctcaggtggaggcggttcaggcggaggtggcagcggcggtggcggatcgtctgagctgactcaggaccctgctgtgtctgt
ggccttgggacagacagtcaggatcacatgccaaggagacagcctcagaagctattatgcaagctggtaccagcagaagcc
aggacaggcccctgtacttgtcatctatggtaaaaacaaccggccctcagggatcccagaccgattctctggctccagctcagga
aacacagcttccttgaccatcactggggctcaggcggaagatgaggctgactattactgtaactcccgggacagcagtggtaac
catgtggtattcggcggagggaccaagctgaccgtcctaggtgacgtacgcgagcccaaatcttctgacaaaactcacacatgc
ccaccgtgcccagcacctgaactcctgggtggaccgtcagtcttcctcttccccccaaaacccaaaggacaccctcatgatctccc
ggacccctgaggtcacatgcgtggtggtggacgtgagccacgaagaccctgaggtcaagttcaactggtacgtggacggcgtg
gaggtgcataatgccaagacaaagccgcgggaggagcagtacaacagcacgtaccgtgtggtcagcgtcctcaccgtcctgc
accaggactggctgaatggcaaggagtacaagtgcaaggtctccaacaaagccctcccagcccccatcgagaaaaccatctc
caaagccaaagggcagccccgagaaccacaggtgtacaccctgcccccatcccgggatgagctgaccaagaaccaggtca
gcctgacctgcctggtcaaaggcttctatccaagcgacatcgccgtggagtgggagagcaatgggcagccggagaacaacta
caagaccacgcctcccgtgctggactccgacggctccttcttcctctacagcaagctcaccgtggacaagagcaggtggcagc
aggggaacgtcttctcatgctccgtgatgcatgaggctctgcacaaccactacacgcagaagagcctctccctgtctccgggtaa
atga (SEQ ID NO: 222)
>HER082_Protein_leader-stop
MEAPAQLLFLLLLWLPDTTGQVQLQESGGGVVQPGGSLRLSCAASGFTFSGYGMHWVRQ
APGKGLEWVASVRNDGSNTYYTDSVKDRFTISRDNTKNTLYLQMNSLRAEDTAVYYCAKS
RRVMYGTSYYFDYWGRGTLVTVSSGGGGSGGGGSGGGGSSELTQDPAVSVALGQTVRIT
CQGDSLRSYYASWYQQKPGQAPVLVIYGKNNRPSGIPDRFSGSSSGNTASLTITGAQAED
EADYYCNSRDSSGNHVVFGGGTKLTVLGDVREPKSSDKTHTCPPCPAPELLGGPSVFLFP
PKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCS
VMHEALHNHYTQKSLSLSPGK (SEQ ID NO: 223)
>HER083_CDS
atggaagcaccagcgcagcttctcttcctcctgctactctggctcccagataccaccggtcaggtgcagctgcaggagtcgggcg
caggactggtgaagccttcggggaccctgtccctcacctgcgctgtctctggtggctccatcagcagtggtaactggtggagttgg
gtccgccagcccccagggaaggggctggagtggattggggaaatctctcatagtgggagcaccaactacaacccgtccctca
agagtcgagtcaccatatcagtagacaagtccaagaaccagttctccctgaacctgagttctgtgaccgccgcagacacggcc
gtgtattactgtgcgagagtaaggggtacggtgggggatacacggggacctgactactggggccagggaaccctggtcaccgt
ctcgagtggtggaggcggttcaggcggaggtggcagcggcggtggcggatcgtctgagctgactcaggaccctgctgtgtctgt
ggccttgggacagacagtcaggatcacatgccaaggagacagcctcagaagctattatgcaagctggtaccagcagaagcc
aggacaggcccctgtacttgtcatctatggtaaaaacaaccggccctcagggatcccagaccgattctctggctccagctcagga
aacacagcttccttgaccatcactggggctcaggcggaagatgaggctgactattactgtaactcccgggacagcagtggtaac
catgtggtattcggcggagggaccaagctgaccgtcctaggtgacgtacgcgagcccaaatcttctgacaaaactcacacatgc
ccaccgtgcccagcacctgaactcctgggtggaccgtcagtcttcctcttccccccaaaacccaaggacaccctcatgatctccc
ggacccctgaggtcacatgcgtggtggtggacgtgagccacgaagaccctgaggtcaagttcaactggtacgtggacggcgtg
gaggtgcataatgccaagacaaagccgcgggaggagcagtacaacagcacgtaccgtgtggtcagcgtcctcaccgtcctgc
accaggactggctgaatggcaaggagtacaagtgcaaggtctccaacaaagccctcccagcccccatcgagaaaaccatctc
caaagccaaagggcagccccgagaaccacaggtgtacaccctgcccccatcccgggatgagctgaccaagaaccaggtca
gcctgacctgcctggtcaaaggcttctatccaagcgacatcgccgtggagtgggagagcaatgggcagccggagaacaacta
caagaccacgcctcccgtgctggactccgacggctccttcttcctctacagcaagctcaccgtggacaagagcaggtggcagc
aggggaacgtcttctcatgctccgtgatgcatgaggctctgcacaaccactacacgcagaagagcctctccctgtctccgggtaa
atga (SEQ ID NO: 224)
>HER083_Protein_leader-stop
MEAPAQLLFLLLLWLPDTTGQVQLQESGAGLVKPSGTLSLTCAVSGGSISSGNWWSWVRQ
PPGKGLEWIGEISHSGSTNYNPSLKSRVTISVDKSKNQFSLNLSSVTAADTAVYYCARVRGT
VGDTRGPDYWGQGTLVTVSSGGGGSGGGGSGGGGSSELTQDPAVSVALGQTVRITCQG
DSLRSYYASWYQQKPGQAPVLVIYGKNNRPSGIPDRFSGSSSGNTASLTITGAQAEDEADY
YCNSRDSSGNHVVFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHE
ALHNHYTQKSLSLSPGK (SEQ ID NO: 225)
>HER084_CDS
atggaagcaccagcgcagcttctcttcctcctgctactctggctcccagataccaccggtgaggtgcagctggtgcagtctggggg
aggcctggtcaagcctggggggtccctgagactctcctgtgcagcgtctggattcaccttcagtagctatgggatgcactgggtcc
gccaggctccaggcaaggggctggagtgggtggcaggtattttttatgatggaggtaataaatactatgcagactccgtgaaggg
ccgattcaccatctccagagacaattccaagaacacgctgtatctgcaaatgaacagcctgagagctgaggacacggctgtgta
ttactgtgcgagagataggggctactactacatggacgtctggggcaaagggaccacggtcaccgtctcctcaggtggaggcg
gttcaggcggaggtggctctggcggtggcggatcgcagtctgtgttgacgcagccgccctcagtgtctggggccccaggacaga
gggtcaccatctcctgcactgggagaagctccaacatcggggcgggtcatgatgtacactggtaccagcaacttccaggaaca
gcccccaaactcctcatctatggtgacagcaatcggccctcaggggtccctgaccgattctctggctccaggtctggcacctcagc
ctccctggccatcactgggctccaggctgaagatgaggctgattattactgccagtcctatgacagcagcctgaggggttcggtatt
cggcggagggaccaaggtcaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcat
aatgccaagacaaagccgcgggaggagcagtacaacagcacgtaccgtgtggtcagcgtcctcaccgtcctgcaccaggac
tggctgaatggcaaggagtacaagtgcaaggtctccaacaaagccctcccagcccccatcgagaaaaccatctccaaagcca
aagggcagccccgagaaccacaggtgtacaccctgcccccatcccgggatgagctgaccaagaaccaggtcagcctgacct
gcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 226)
>HER084_Protein_leader-stop
MEAPAQLLFLLLLWLPDTTGEVQLVQSGGGLVKPGGSLRLSCAASGFTFSSYGMHWVRQA
PGKGLEWVAGIFYDGGNKYYADSVKGRFTISRDNSKNTLYLQMNSLRAEDTAVYYCARDR
GYYYMDVWGKGTTVTVSSGGGGSGGGGSGGGGSQSVLTQPPSVSGAPGQRVTISCTGR
SSNIGAGHDVHWYQQLPGTAPKLLIYGDSNRPSGVPDRFSGSRSGTSASLAITGLQAEDEA
DYYCQSYDSSLRGSVFGGGTKVTVLGDVREPKSSDKTHTCPPCPAPELLGGPSVFLFPPK
PKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVM
HEALHNHYTQKSLSLSPGK (SEQ ID NO: 227)
>HER085_CDS
atggaagcaccagcgcagcttctcttcctcctgctactctggctcccagataccaccggtgaggtgcagctgttggagtctggggg
aggcttggtacagcctggggggtccctgagactctcctgtgcagcctctggattcacctttagcagctatgccatgagctgggtccg
ccaggctccagggaaggggctggagtgggtctcagctattagtggtagtggtggtagcacatactacgcagactccgtgaaggg
ccggttcaccatctccagagacaattccaagaacacgctgtatctgcaaatgaacagcctgagagccgaggacacggccgtgt
attactgtgcgagaggcgggagtgggagtgactactggggccaggggacaatggtcaccgtctcgagtggaggcggcggttc
aggcggaggtggctctggcggtggcggaagtgcacttaattttatgctgactcagccccactctgtgtcggggtctccggggaag
acggtaaccatctcctgcacccgcagcagtggctacattgacagcaagtatgtgcagtggtaccagcagcgcccgggcagtgc
ccccaccactgtgatctatgaggataaccgaagaccctctggggtccctgatcggttctctggctccatcgacagctcctccaactc
tgcctccctcaccatctctggactggagactgaggacgaggctgactattactgtcagtcttatgatgacaccaatgtggtgttcggc
ggagggaccaaggtcaccgtcctaggtgacgtacgcgagcccaaatcttctgacaaaactcacacatgcccaccgtgcccag
cacctgaactcctgggtggaccgtcagtcttcctcttccccccaaaacccaaggacaccctcatgatctcccggacccctgaggt
cacatgcgtggtggtggacgtgagccacgaagaccctgaggtcaagttcaactggtacgtggacggcgtggaggtgcataatg
ccaagacaaagccgcgggaggagcagtacaacagcacgtaccgtgtggtcagcgtcctcaccgtcctgcaccaggactggct
gaatggcaaggagtacaagtgcaaggtctccaacaaagccctcccagcccccatcgagaaaaccatctccaaagccaaag
ggcagccccgagaaccacaggtgtacaccctgcccccatcccgggatgagctgaccaagaaccaggtcagcctgacctgcct
ggtcaaaggcttctatccaagcgacatcgccgtggagtgggagagcaatgggcagccggagaacaactacaagaccacgcc
tcccgtgctggactccgacggctccttcttcctctacagcaagctcaccgtggacaagagcaggtggcagcaggggaacgtcttc
tcatgctccgtgatgcatgaggctctgcacaaccactacacgcagaagagcctctccctgtctccgggtaaatga (SEQ ID NO: 228)
>HER085_Protein_leader-stop
MEAPAQLLFLLLLWLPDTTGEVQLLESGGGLVQPGGSLRLSCAASGFTFSSYAMSWVRQA
PGKGLEWVSAISGSGGSTYYADSVKGRFTISRDNSKNTLYLQMNSLRAEDTAVYYCARGG
SGSDYWGQGTMVTVSSGGGGSGGGGSGGGGSALNFMLTQPHSVSGSPGKTVTISCTRS
SGYIDSKYVQWYQQRPGSAPTTVIYEDNRRPSGVPDRFSGSIDSSSNSASLTISGLETEDE
ADYYCQSYDDTNVVFGGGTKVTVLGDVREPKSSDKTHTCPPCPAPELLGGPSVFLFPPKP
KDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVM
HEALHNHYTQKSLSLSPGK (SEQ ID NO: 229)
>HER086_CDS
atggaagcaccagcgcagcttctcttcctcctgctactctggctcccagataccaccggtggggtgcagctggtggagtctgggg
gaggcctggtcaagcctggggggtccctgagactctcctgtgcagcctctggattcaccttcagtagctataacatgaactgggtc
cgccaggctccagggaagggactggagtgggtctcagctattagtggtagtggtggtagcacatactacgcagactccgtgacg
ggccggttcaccatctccagagacaattccaagaacacgctgtatctgcaaatgaacagcctgagagccgaggacacggccg
tatattactgtgcgaaagataccagtggctggtacggggacggtatggacgtctggggccggggaaccctggtcaccgtctcga
gtggtggaggcggttcaggcggaggtggcagcggcggtggcggatcggacatccagatgacccagtctccttccaccctgtctg
catctattggagacagagtcaccatcacctgccgggccagtgagggtatttatcactggttggcctggtatcagcagaagccagg
gaaagcccctaaactcctgatctataaggcctctagtttagccagtggggccccatcaaggttcagcggcagtggatcagggac
agatttcactctcaccatcagcagcctgcagcctgatgattttgcaacttattactgccaacaatatagtaattatccgctcactttcgg
cggagggaccaagctggagatcaaacgtgacgtacgcgagcccaaatcttctgacaaaactcacacatgcccaccgtgccca
gcacctgaactcctgggtggaccgtcagtcttcctcttccccccaaaacccaaggacaccctcatgatctcccggacccctgagg
tcacatgcgtggtggtggacgtgagccacgaagaccctgaggtcaagttcaactggtacgtggacggcgtggaggtgcataatg
ccaagacaaagccgcgggaggagcagtacaacagcacgtaccgtgtggtcagcgtcctcaccgtcctgcaccaggactggct
gaatggcaaggagtacaagtgcaaggtctccaacaaagccctcccagcccccatcgagaaaaccatctccaaagccaaag
ggcagccccgagaaccacaggtgtacaccctgcccccatcccgggatgagctgaccaagaaccaggtcagcctgacctgcct
ggtcaaaggcttctatccaagcgacatcgccgtggagtgggagagcaatgggcagccggagaacaactacaagaccacgcc
tcccgtgctggactccgacggctccttcttcctctacagcaagctcaccgtggacaagagcaggtggcagcaggggaacgtcttc
tcatgctccgtgatgcatgaggctctgcacaaccactacacgcagaagagcctctccctgtctccgggtaaatga (SEQ ID NO: 230)
>HER086_Protein_leader-stop
MEAPAQLLFLLLLWLPDTTGGVQLVESGGGLVKPGGSLRLSCAASGFTFSSYNMNWVRQA
PGKGLEWVSAISGSGGSTYYADSVTGRFTISRDNSKNTLYLQMNSLRAEDTAVYYCAKDTS
GWYGDGMDVWGRGTLVTVSSGGGGSGGGGSGGGGSDIQMTQSPSTLSASIGDRVTITC
RASEGIYHWLAWYQQKPGKAPKLLIYKASSLASGAPSRFSGSGSGTDFTLTISSLQPDDFAT
YYCQQYSNYPLTFGGGTKLEIKRDVREPKSSDKTHTCPPCPAPELLGGPSVFLFPPKPKDT
LMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEA
LHNHYTQKSLSLSPGK (SEQ ID NO: 231)
>HER087_CDS
atggaagcaccagcgcagcttctcttcctcctgctactctggctcccagataccaccggtgaggtgcagctgttggagtctggggg
aggcttggtacagcctggggggtccctgagactctcctgtgcagcctctggattcacctttagcagctatgccatgagctgggtccg
ccaggctccagggaaggggctggagtgggtctcagctattagtggtagtggtggtagcacatactacgcagactccgtgaaggg
ccggttcaccatctccagagacaattccaagaacacgctgtatctgcaaatgaacagcctgagagccgaggacacggccgtgt
attactgtgcgagagtcagcgggagccactttccattctttgactcctggggccaggggacaatggtcaccgtctcgagtggaggc
ggcggttcaggcggaggtggctctggcggtggcggaagtgcacagtctgtgctgactcagccaccctcggtgtcagtggcccca
ggacagacggccagaattacctgtgggggagacaagattggacataaaagtgtgcattggtatcagcagaagccaggccag
gcccctgtgttgctcgtctatgatgataggaagcggccctcagggatccctgagcgattctctggctccaactctgggaacacggc
caccctgaccatcagcagggtcgaggccggggatgaggctgcctatcactgtcaggtgtgggatagaagtagtgacccttatgt
cttcggaactgggaccaaggtcaccgtcctaggtgacgtacgcgagcccaaatcttctgacaaaactcacacatgcccaccgtg
cccagcacctgaactcctgggtggaccgtcagtcttcctcttccccccaaaacccaaggacaccctcatgatctcccggacccct
gaggtcacatgcgtggtggtggacgtgagccacgaagaccctgaggtcaagttcaactggtacgtggacggcgtggaggtgca
taatgccaagacaaagccgcgggaggagcagtacaacagcacgtaccgtgtggtcagcgtcctcaccgtcctgcaccagga
ctggctgaatggcaaggagtacaagtgcaaggtctccaacaaagccctcccagcccccatcgagaaaaccatctccaaagcc
aaagggcagccccgagaaccacaggtgtacaccctgcccccatcccgggatgagctgaccaagaaccaggtcagcctgac
ctgcctggtcaaaggcttctatccaagcgacatcgccgtggagtgggagagcaatgggcagccggagaacaactacaagacc
acgcctcccgtgctggactccgacggctccttcttcctctacagcaagctcaccgtggacaagagcaggtggcagcaggggaa
cgtcttctcatgctccgtgatgcatgaggctctgcacaaccactacacgcagaagagcctctccctgtctccgggtaaatga (SEQ ID NO: 232)
>HER087_Protein_leader-stop
MEAPAQLLFLLLLWLPDTTGEVQLLESGGGLVQPGGSLRLSCAASGFTFSSYAMSWVRQA
PGKGLEWVSAISGSGGSTYYADSVKGRFTISRDNSKNTLYLQMNSLRAEDTAVYYCARVS
GSHFPFFDSWGQGTMVTVSSGGGGSGGGGSGGGGSAQSVLTQPPSVSVAPGQTARITC
GGDKIGHKSVHWYQQKPGQAPVLLVYDDRKRPSGIPERFSGSNSGNTATLTISRVEAGDE
AAYHCQVWDRSSDPYVFGTGTKVTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGK (SEQ ID NO: 233)
>HER_SMIPs_huVk3_Leader_CDS
atggaagcaccagcgcagcttctcttcctcctgctactctggctcccagataccaccggt (SEQ ID NO: 234)
>HER_SMIPs_huVk3_Leader_Protein
MEAPAQLLFLLLLWLPDTTG (SEQ ID NO: 235)
>HER_SMIPs_G4Sx3_Linker_CDS
ggaggcggcggttcaggcggaggtggctctggcggtggcggaagt (SEQ ID NO: 236)
>HER_SMIPs_G4Sx3_Linker_Protein
GGGGSGGGGSGGGGS (SEQ ID NO: 237)
>HER_SMIPs_SCCP_Hinge_CDS
gagcccaaatcttctgacaaaactcacacatgcccaccgtgccca (SEQ ID NO: 238)
>HER_SMIPs_SCCP_Hinge_Protein
EPKSSDKTHTCPPCP (SEQ ID NO: 239)
>HER_SMIP_Fc-Stop_CDS
gacgtacgcgagcccaaatcttctgacaaaactcacacatgcccaccgtgcccagcacctgaactcctgggtggaccgtcagt
cttcctcttccccccaaaacccaaggacaccctcatgatctcccggacccctgaggtcacatgcgtggtggtggacgtgagocac
gaagaccctgaggtcaagttcaactggtacgtggacggcgtggaggtgcataatgccaagacaaagccgcgggaggagcag
tacaacagcacgtaccgtgtggtcagcgtcctcaccgtcctgcaccaggactggctgaatggcaaggagtacaagtgcaaggt
ctccaacaaagccctcccagcccccatcgagaaaaccatctccaaagccaaagggcagccccgagaaccacaggtgtaca
ccctgcccccatcccgggatgagctgaccaagaaccaggtcagcctgacctgcctggtcaaaggcttctatccaagcgacatc
gccgtggagtgggagagcaatgggcagccggagaacaactacaagaccacgcctcccgtgctggactccgacggctccttct
tcctctacagcaagctcaccgtggacaagagcaggtggcagcaggggaacgtcttctcatgctccgtgatgcatgaggctctgc
acaaccactacacgcagaagagcctctccctgtctccgggtaaatga (SEQ ID NO: 240)
>HER_SMIP_Fc_Stop_Protein
DVREPKSSDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK (SEQ ID NO: 241)
Her2_S1R3B1_DP47_3A2
V_{H} with CDR1, CDR2 and CDR3 underlined
EVQLLESGGGLVQPGGSLRLSCAASGFTFSSYAMSWVRQTPGKGLEWVSAISGSGGSTYYAN
SVKGRFTISRDNSKNTLYLQMNSLRAEDTAVYYCARGGGYNPFDSWGQGTMVTVSS (SEQ ID NO: 251)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGAC
TCCAGGGAAGGGGCTGGAGTGGGTCTCAGCTATTAGTGGTAGTGGTGGTAGCACATACT
ACGCAAACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGCGGGG
GGGAGGCTACAACCCTTTTGACTCCTGGGGCCAGGGGACAATGGTCACCGTCTCGAGT (SEQ ID NO: 252)
V_{L} with CDR1, CDR2 and CDR3 underlined
QSALTQPASVSGSPGQSITISCTGTGSDVGGYNYVSWYQQHPGKAPKLMIYEVINRPSGISNR
FSGSKSGNTASLTISGLQAEDEADYYCGSYSSSSTLVFGGGTKLTVL (SEQ ID NO: 253)
CAGTCTGCCCTGACTCAGCCTGCCTCCGTGTCTGGGTCTCCTGGACAGTCGATCACCATCT
CCTGCACTGGAACCGGCAGTGACGTTGGTGGTTATAACTATGTCTCCTGGTACCAACAGC
ACCCAGGCAAAGCCCCCAAACTCATGATTTATGAGGTCATTAATCGGCCCTCAGGGATTT
CTAATCGCTTCTCTGGCTCCAAGTCTGGCAACACGGCCTCCCTGACCATCTCTGGGCTCC
AGGCTGAGGACGAGGCTGATTATTACTGCGGCTCATATTCAAGCAGCAGCACTCTTGTAT
TCGGCGGAGGGACCAAGCTGACCGTCCTA (SEQ ID NO: 254)
Her2_S1R3A1_DP47_11B7
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAGAADYSNYFDFWGQGTMVTVSS (SEQ ID NO: 255)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGGGAGC
GGCGGACTACAGTAATTACTTTGACTTTTGGGGCCAAGGGACAATGGTCACCGTCTCGAGT (SEQ ID NO: 256)
V_{L} with CDR1, CDR2 and CDR3 underlined
QSVLTQPPSVSGAPGQRVTISCTGSSSNIGAGYDVHWYQQLPGAAPKLLIYGNINRPSGVPDR
FSGSKSGTSASLAITGLQAEDEGDYYCQSYDRSLSAKLFGGGTKVTVL (SEQ ID NO: 257)
CAGTCTGTGCTGACTCAGCCACCCTCAGTGTCTGGGGCCCCAGGGCAGAGGGTCACCATC
TCCTGCACTGGGAGCAGCTCCAACATCGGGGCAGGTTATGATGTACACTGGTACCAGCA
ACTTCCAGGAGCAGCCCCCAAACTCCTCATCTATGGGAACATCAATCGGCCCTCAGGGGT
CCCTGACCGATTCTCTGGCTCCAAGTCTGGCACCTCAGCCTCCCTGGCCATCACTGGGCT
CCAGGCTGAGGATGAGGGTGATTATTACTGCCAGTCCTATGACAGAAGCCTGAGTGCTA
AGCTGTTCGGCGGAGGGACCAAGGTCACCGTCCTA (SEQ ID NO: 258)
Her2_S1R3A1_DP47_11D1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNALYLQMNSLRAEDTAVYYCARDLGIDPLWSGYYTPLDYWGRGTM
VTVSS (SEQ ID NO: 259)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACGCGCTG
TATCTGCAAATGAACAGCCTGAGAGCCGAGGACACGGCCGTGTATTACTGTGCGAGAGA
TCTGGGAATAGACCCCCTTTGGAGTGGTTATTACACACCCCTTGACTATTGGGGCCGGGG
GACAATGGTCACCGTCTCGAGT (SEQ ID NO: 260)
V_{L} with CDR1, CDR2 and CDR3 underlined
SSELTQDPALSVALGQTVRITCQGDSLGGFHASWYQEKPGQAPVFVLYGKNNRPSGIPDRFS
GSTSGNTAALTITGAQAEDEADYYCSSRDRSGNHRVFGGGTKLTVL (SEQ ID NO: 261)
TCTTCTGAGCTGACTCAGGACCCTGCTCTGTCGGTGGCCTTGGGACAGACAGTCAGGATC
ACATGTCAAGGGGACAGCCTCGGAGGCTTTCATGCAAGCTGGTACCAGGAGAAGCCAGG
ACAGGCCCCTGTATTTGTCCTCTATGGTAAAAACAACCGGCCCTCAGGGATCCCAGACCG
ATTCTCTGGCTCCACCTCAGGTAACACAGCTGCCCTGACCATCACTGGGGCTCAGGCGGA
AGATGAGGCTGACTATTACTGTAGCTCCCGGGACAGAAGTGGTAACCATCGCGTCTTCGG
CGGAGGGACCAAGCTGACCGTCCTA (SEQ ID NO: 262)
Her2_S1R3A1_DP47_7F3
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARDRALVGATRTFGYWGQGTTVTVSS (SEQ ID NO: 263)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGA
TCGAGCCCTAGTGGGAGCTACTCGAACTTTTGGCTACTGGGGGCAGGGGACCACGGTCA
CCGTCTCGAGT (SEQ ID NO: 264)
V_{L} with CDR1, CDR2 and CDR3 underlined
QAVLTQPSSVSGAPGQRVTISCTGSSSNIGAGYDVHWYQQLPGTAPKLLIYGDTNRPSGVPDR
FSGSKSGTSASLAITGLQAEDEADYYCQSFDSSLSGSVFGGGTKLTVL (SEQ ID NO: 265)
CAGGCTGTGCTGACTCAGCCGTCCTCAGTGTCTGGGGCCCCAGGGCAGAGGGTCACCATC
TCCTGCACTGGGAGCAGCTCCAACATCGGGGCAGGTTATGATGTACACTGGTACCAGCA
GCTTCCAGGAACAGCCCCCAAACTCCTCATCTATGGTGACACCAATCGGCCCTCAGGGGT
CCCTGACCGATTCTCTGGCTCCAAGTCTGGCACCTCAGCCTCCCTGGCCATCACTGGGCT
CCAGGCTGAGGATGAGGCTGATTATTACTGCCAGTCCTTTGACAGCAGCCTCAGTGGTTC
GGTGTTCGGCGGAGGGACCAAGCTGACCGTCCTA (SEQ ID NO: 266)
Her2_S1R2B_DP47_4E3
V_{H} with CDR1, CDR2 and CDR3 underlined
EVLLLESGGGLVQPGGSLRLSCAASGFTFSSYAMSWVRQAPGKGLEWVSAISGSGGSTYYAD
SAKGRFTISRDNSKNTLYLQMNSLRAEDTAVYYCARDRVSNWNYYGQDSYFDYWGQGTM
VTVSS (SEQ ID NO: 267)
GAGGTGCTGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATCAGTGGTAGTGGTGGTAGCACATACT
ACGCAGACTCCGCGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGGGA
CAGGGTCTCTAACTGGAACTACTACGGCCAGGACAGCTACTTTGACTACTGGGGCCAAG
GGACAATGGTCACCGTCTCGAGT (SEQ ID NO: 268)
V_{L} with CDR1, CDR2 and CDR3 underlined
QAVLTQPPSASGTPGQRVTISCSGSSSNIGSNYVYWYQQLPGTAPKVLIYRNNQRPSGVPDRF
SGSKSGTSASLAISGLRSEDEADYYCASWDGSLSGPVFGGGTKLTVL (SEQ ID NO: 269)
CAGGCTGTGCTGACTCAGCCACCCTCAGCGTCTGGGACCCCCGGGCAGAGGGTCACCAT
CTCTTGTTCTGGAAGCAGCTCCAACATCGGAAGTAATTATGTATACTGGTACCAGCAACT
CCCAGGAACGGCCCCCAAAGTCCTCATCTATAGGAATAATCAGCGGCCCTCAGGGGTCC
CTGACCGATTCTCTGGCTCCAAGTCTGGCACCTCAGCGTCCCTGGCCATCAGTGGGCTCC
GGTCCGAGGATGAGGCTGATTATTACTGTGCATCATGGGATGGCAGCCTGAGTGGTCCG
GTGTTCGGCGGAGGGACCAAGCTGACCGTCCTA (SEQ ID NO: 270)
Her2_S1R3C1_DP47_2G2
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARDRVSNWNYYGQDSYFGYWGQGT
MVTVSS (SEQ ID NO: 271)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGGGA
CAGGGTCTCTAACTGGAACTACTACGGCCAGGACAGCTACTTTGGCTACTGGGGCCAGG
GGACAATGGTCACCGTCTCGAGT (SEQ ID NO: 272)
V_{L} with CDR1, CDR2 and CDR3 underlined
SYELTQPPSASGTPGQRVTISCSGSSSNIGSNTVTWYQQLPGTAPQLLFHNNDQRPSGVPDRFS
GSKSGTSGSLAISGLQSEDEADYYCSAWDDGLNAVIFGGGTKLTVL (SEQ ID NO: 273)
TCCTATGAGCTGACTCAGCCACCCTCAGCGTCTGGGACCCCCGGGCAGAGGGTCACCATC
TCTTGTTCTGGAAGCAGCTCCAACATCGGAAGTAATACTGTAACCTGGTACCAGCAGCTC
CCAGGAACGGCCCCCCAACTCCTCTTCCATAATAATGACCAGCGGCCCTCAGGGGTCCCT
GACCGATTCTCTGGCTCCAAGTCTGGCACCTCAGGCTCCCTGGCCATCAGTGGGCTGCAG
TCTGAGGATGAGGCTGATTATTACTGTTCAGCATGGGATGACGGCCTGAATGCTGTAATA
TTCGGCGGAGGGACCAAGCTGACCGTCCTA (SEQ ID NO: 274)
Her2_S1R3A1_DP47_11H6
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARDRVYDFWSGYYTRYNWFDPWGRG
TTVTVSS (SEQ ID NO: 275)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGA
TCGCGTTTACGATTTTTGGAGTGGTTATTATACGAGGTACAACTGGTTCGACCCCTGGGG
GCGAGGGACCACGGTCACCGTCTCGAGT (SEQ ID NO: 276)
V_{L} with CDR1, CDR2 and CDR3 underlined
QAVLTQPSSASGTPGQRVTISCSGSSSNIGSNYVYWYQQLPGTAPKLLIYRNNQRPSGVPDRF
SGSKSGTSASLAISGLRSEDEADYYCAAWDDSLSSPVFGGGTKVTVL (SEQ ID NO: 277)
CAGGCTGTGCTGACTCAGCCGTCCTCAGCGTCTGGGACCCCCGGGCAGAGGGTCACCATC
TCTTGTTCTGGAAGCAGCTCCAACATCGGAAGTAATTATGTATACTGGTACCAGCAGCTC
CCAGGAACGGCCCCCAAACTCCTCATCTATAGGAATAATCAGCGGCCCTCANGGGTCCCT
GACCGATTCTCTGGCTCCAAGTCTGGCACCTCAGCCTCCCTGGCCATCAGTGGGCTCCGG
TCCGAGGATGAGGCTGATTATTACTGTGCAGCATGGGATGACAGCCTGAGTAGTCCGGT
GTTCGGCGGAGGGACCAAGGTCACCGTCCTA (SEQ ID NO: 278)
Her2_S1R3A1_BMV_3B1
V_{H} with CDR1, CDR2 and CDR3 underlined
EVQLVQSGGGLVQPGGSLRLSCAASGFTFSSYPMHWVRQAPGKGLEWVAVVSFDGSKKYS
ADSVKGRFTISRDISKNTLYLQMNSLRAEDTAVYYCAKDRYDSGTFYYGMDVWGRGTLVT
VSS (SEQ ID NO: 279)
GAGGTGCAGCTGGTGCAGTCTGGGGGAGGCTTGGTACAGCCTGGGGGATCCCTGAGACT
CTCCTGTGCAGCCTCTGGATTCACCTTTAGCAGCTATCCCATGCACTGGGTCCGCCAGGC
TCCAGGCAAGGGGCTGGAGTGGGTGGCAGTTGTATCGTTCGATGGATCTAAGAAATACT
CTGCAGACTCCGTGAAGGGCCGATTCACCATCTCCAGAGACATCTCCAAGAACACGCTGT
ATCTGCAAATGAACAGCCTGAGAGCTGAGGACACGGCTGTATATTACTGTGCGAAAGAT
CGCTATGATTCGGGGACTTTCTACTACGGCATGGACGTCTGGGGCCGGGGCACCCTGGTC
ACCGTCTCGAGT (SEQ ID NO: 280)
V_{L} with CDR1, CDR2 and CDR3 underlined
QSALTQPASVSGSRGQSITISCTGTTGDVGGYDYVSWYQQHPGRAPKLLIYGNSNRPSGVPD
RFSASKSGNTASLTISGLQAEDEADYFCSTYAPPGIIMFGGGTKLTVL (SEQ ID NO: 281)
CAGTCTGCCCTGACTCAGCCTGCCTCCGTGTCTGGATCTCGTGGACAGTCGATCACCATC
TCCTGCACTGGAACCACTGGTGACGTTGGTGGTTATGACTATGTCTCCTGGTACCAACAG
CACCCAGGCAGAGCCCCCAAACTCCTCATCTATGGTAACAGCAATCGGCCCTCAGGGGT
CCCTGATCGCTTCTCTGCCTCCAAGTCCGGCAATACGGCCTCCCTGACCATCTCTGGACTC
CAGGCTGAGGATGAGGCTGATTATTTCTGCAGCACATATGCACCCCCCGGTATTATTATG
TTCGGCGGAGGGACCAAGCTGACCGTCCTA (SEQ ID NO: 282)
Her2_S1R3A1_DP47_6B9
V_{H} with CDR1, CDR2 and CDR3 underlined
EVQLLESGGGLVQPGGSLRLSCAASGFTFSSYAISWVRQAPGKGLEWVSAISGSGGSTYYAD
SVKGRFTISRDNSKNTLYLQMNSLRAEDTAVYYCARDSSRVGAYLVFDYWGRGTMVTVSS (SEQ ID NO: 283)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATAAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGA
TTCGAGTAGGGTGGGAGCTTATCTGGTGTTTGACTACTGGGGCCGGGGGACAATGGTCAC
CGTCTCGAGT (SEQ ID NO: 284)
V_{L} with CDR1, CDR2 and CDR3 underlined
QSVLTQPPSVSGAPGQRVTISCTGSSSNIGAGYDVHWYQQLPGTAPKLLIYGNSKRPSGVPDR
FSGSKSGTSASLALTGLQAEDEADYYCQSYDSSLSGYVFGTGTKVTVL (SEQ ID NO: 285)
CAGTCTGTGCTGACGCAGCCGCCCTCAGTGTCTGGGGCCCCAGGGCAGAGGGTCACCAT
CTCCTGCACTGGGAGCAGCTCCAACATCGGGGCAGGTTATGATGTACACTGGTACCAGC
AGCTTCCAGGAACAGCCCCCAAACTCCTCATCTATGGTAACAGCAAACGCCCCTCAGGG
GTCCCTGACCGATTCTCTGGCTCCAAGTCTGGCACCTCAGCCTCCCTGGCCCTCACTGGG
CTCCAGGCTGAGGATGAGGCTGATTATTACTGCCAGTCCTATGACAGCAGCCTGAGTGGT
TATGTCTTCGGAACTGGGACCAAGGTCACCGTCCTA (SEQ ID NO: 286)
Her2_S1R2A_CS_10B8
V_{H} with CDR1, CDR2 and CDR3 underlined
QMQLVQSGAEVKKPGASVKVSCKASGYTFTSYYIHWVRQAPGQGPEWMGIILPSGGSTSYA
QEFQGRLSMTRDTSTSTVYMELSDLRSDDTAIYYCARDYDRSAYLDIWGRGTMVTVSS (SEQ ID NO: 287)
CAGATGCAGCTGGTGCAGTCTGGGGCTGAGGTGAAGAAGCCTGGGGCCTCAGTGAAGGT
TTCCTGCAAGGCATCTGGATACACCTTCACCAGCTACTATATACACTGGGTGCGACAGGC
CCCTGGACAGGGCCCTGAGTGGATGGGAATAATCCTCCCTAGTGGTGGCAGCACCAGCT
ACGCACAGGAGTTCCAGGGCAGACTCTCCATGACCAGGGACACGTCCACGAGCACAGTG
TACATGGAGCTGAGCGACCTGAGATCTGACGACACGGCCATTTATTATTGTGCGAGAGA
CTATGATAGGAGTGCTTATCTTGATATCTGGGGCCGAGGGACAATGGTCACCGTCTCGAGT (SEQ ID NO: 288)
V_{L} with CDR1, CDR2 and CDR3 underlined
QSVLTQPPSASGTPGQRVTISCSGSSSNIGRNTVNWYKQFPGTAPKLLIYSDNKRPSGIPDRFS
GSKSGTSASLAISGLQSGDEADYYCAAWDDSLNGHVVFGGGTKLTVL (SEQ ID NO: 289)
CAGTCTGTGTTGACGCAGCCGCCCTCAGCGTCTGGGACCCCCGGGCAGAGGGTCACCATC
TCTTGTTCTGGAAGCAGCTCCAACATCGGAAGAAATACTGTAAACTGGTACAAGCAGTTC
CCAGGAACGGCCCCCAAACTCCTCATCTATAGTGATAATAAGCGGCCCTCAGGGATCCCT
GACCGATTCTCTGGCTCCAAGTCTGGCACCTCAGCCTCCCTGGCCATCAGTGGGCTCCAG
TCTGGGGATGAGGCTGATTATTACTGTGCCGCATGGGATGACAGCCTGAATGGCCATGTG
GTATTCGGCGGAGGGACCAAGCTGACCGTCCTA (SEQ ID NO: 290)
Her2_S1R3A1_DP47_7A6
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNGLRVEDTAVYYCAKELVSRGSLTFDYWGKGTMVTVSS (SEQ ID NO: 291)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACGGCCTGAGAGTCGAGGACACGGCCGTGTATTACTGTGCGAAAGA
ATTGGTCAGTAGAGGGAGCCTCACCTTTGACTACTGGGGCAAGGGGACAATGGTCACCG
TCTCGAGT (SEQ ID NO: 292)
V_{L} with CDR1, CDR2 and CDR3 underlined
QSVLTQPPSVSGAPGQGVTISCTGSSSNIGADFAVHWYQQLPGTAPKLLINGSSHRPSGVPDR
FSGSKSGPSASLAITGLQADDEADYFCQSYDYRLNALVFGGGTKLTVL (SEQ ID NO: 293)
CAGTCTGTGTTGACGCAGCCGCCCTCAGTGTCTGGGGCCCCAGGGCAGGGGGTCACCATC
TCCTGCACTGGGAGCAGCTCCAACATCGGGGCAGATTTTGCTGTACACTGGTACCAACAA
CTTCCAGGGACAGCCCCCAAACTCCTCATCAATGGTAGCAGCCATCGGCCCTCAGGGGTC
CCTGACCGATTCTCTGGCTCCAAGTCTGGCCCCTCAGCCTCCCTGGCCATCACTGGGCTCC
AAGCCGACGATGAGGCTGATTATTTTTGCCAGTCCTATGACTACAGACTCAATGCTTTAG
TGTTCGGCGGAGGGACCAAGCTGACCGTCCTA (SEQ ID NO: 294)
Her2_S1R3B2_DP47_2G3
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARGHKMGYFDYWGRGTLVTVSS (SEQ ID NO: 295)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGG
TCACAAAATGGGATACTTTGACTACTGGGGCCGGGGCACCCTGGTCACCGTCTCGAGT (SEQ ID NO: 296)
V_{L} with CDR1, CDR2 and CDR3 underlined
SSELTQDPAVSVALGQSVTITCRGASLSNYYASWYQQRPGQAPLLVVSDNNIRPSGIPDRFSG
SRSGTTASLSITGAQAEDEADYYCHSRASSDTHVRVFGGGTKLTVL (SEQ ID NO: 297)
TCTTCTGAGCTGACTCAGGACCCTGCTGTGTCCGTGGCCTTGGGACAGTCAGTCACCATC
ACGTGTCGGGGAGCCAGCCTCAGCAACTATTATGCAAGCTGGTACCAGCAGAGGCCAGG
ACAAGCCCCTCTACTTGTCGTCTCTGATAACAACATCCGGCCCTCAGGGATCCCAGACCG
ATTCTCTGGCTCCAGGTCAGGAACCACAGCTTCCTTGAGCATCACTGGGGCTCAGGCGGA
AGATGAGGCTGACTATTACTGTCACTCCCGTGCCAGCAGTGACACCCATGTCCGGGTGTT
TGGCGGCGGGACCAAGCTGACCGTCCTA (SEQ ID NO: 298)
Her2_S1R2B_CS_6H11
V_{H} with CDR1, CDR2 and CDR3 underlined
EVQLVQSGAEVKQPGESLKISCKGSGYSFSNYWIGWVRQMPGKGLEWMGIIYPDDSDTRYSP
SFQGQVTISADRSISTAYLQWSSLKASDTATYYCARGNVINGNTNAFDIWGRGTTVTVSS (SEQ ID NO: 299)
GAGGTGCAGCTGGTGCAGTCTGGGGCAGAGGTGAAACAGCCCGGGGAGTCTCTGAAGAT
CTCCTGTAAGGGTTCTGGATACAGCTTTAGCAACTACTGGATCGGCTGGGTGCGCCAGAT
GCCCGGGAAAGGCCTGGAGTGGATGGGGATCATCTATCCTGATGACTCTGATACCAGAT
ACAGCCCGTCCTTCCAAGGCCAGGTCACCATCTCAGCCGACAGGTCCATCAGCACCGCCT
ACCTGCAGTGGAGCAGCCTGAAGGCCTCGGACACCGCCACGTATTACTGTGCGAGAGGA
AATGTTATAAATGGAAATACCAATGCTTTTGATATCTGGGGGCGGGGGACCACGGTCAC
CGTCTCGAGT (SEQ ID NO: 300)
V_{L} with CDR1, CDR2 and CDR3 underlined
QAVVIQEPSFSVSPGGTVTLTCGLSSGSVSTGYYPSWYQQTPGQAPRTLIYNTNSRSSGVPDR
FSGSILGNKAALTITGAQADDESDYYCVLYMGSGISVFGGGTKLTVL (SEQ ID NO: 301)
CAGGCTGTGGTGATCCAGGAGCCATCGTTCTCAGTGTCCCCTGGAGGGACAGTCACACTC
ACTTGTGGCTTGAGCTCTGGCTCAGTCTCTACTGGTTACTACCCCAGCTGGTACCAGCAG
ACCCCAGGCCAGGCTCCACGCACGCTCATCTACAACACAAACAGTCGCTCTTCTGGGGTC
CCTGATCGCTTCTCTGGCTCCATCCTTGGGAACAAAGCTGCCCTCACCATCACGGGGGCC
CAGGCAGATGATGAATCTGATTATTACTGTGTGCTGTATATGGGTAGTGGCATTTCGGTA
TTCGGCGGAGGGACCAAGCTGACCGTCCTA (SEQ ID NO: 302)
Her2_S1R3A1_DP47_10G1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAGGRVGSTAAFDTWGQGTMVTVSS (SEQ ID NO: 303)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGGGAGG
TAGAGTGGGATCTACGGCGGCTTTTGATACATGGGGCCAGGGGACAATGGTCACCGTCT
CGAGT (SEQ ID NO: 304)
V_{L} with CDR1, CDR2 and CDR3 underlined
QSVLTQPPSVSGAPGQRVAISCTGSSSNIGAGYDVHWFQQLPGTAPKLLIFGNKNRPSGVPDR
FSASKSGTAASLAITGLQAEDEGDYYCQSYDSSLSGVIFGRGTKLTVL (SEQ ID NO: 305)
CAGTCTGTGTTAACGCAGCCGCCCTCAGTGTCTGGGGCCCCAGGGCAGAGGGTCGCCAT
ATCCTGTACGGGGAGCAGCTCCAATATTGGGGCAGGTTATGATGTACACTGGTTTCAGCA
ACTTCCAGGAACAGCCCCCAAACTCCTCATCTTTGGTAACAAGAATCGGCCCTCAGGGGT
CCCCGACCGATTCTCTGCCTCTAAGTCTGGCACCGCAGCCTCCCTGGCCATCACTGGGCT
CCAGGCTGAGGATGAGGGTGATTATTACTGCCAGTCCTATGACAGCAGCCTGAGTGGTGT
GATCTTCGGCAGAGGGACCAAGCTGACCGTCCTA (SEQ ID NO: 306)
Her2_S1R3A1_DP47_7C1
V_{H} with CDR1, CDR2 and CDR3 underlined
EVQLLESGGGLVQPGGSLRLSCAASGFTFSSYAMSWVRQAPGKGLEWVSAISGSGGSTYSAD
SVKGRFTISRDNSKNTLYLQMNSLRAEDTAVYYCAGGRVGSTAAFDTWGRGTTVTVSS (SEQ ID NO: 307)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
CCGCAGACTCCGTGAAGGGCCGGTTCACCATCTCCAGAGACAACTCCAAGAACACGCTG
TATCTGCAAATGAACAGCCTGAGAGCCGAGGACACGGCCGTGTATTACTGTGCGGGAGG
TAGAGTGGGATCTACGGCGGCTTTTGATACATGGGGGCGAGGGACCACGGTCACCGTCT
CGAGT (SEQ ID NO: 308)
V_{L} with CDR1, CDR2 and CDR3 underlined
QAVLTQPSSVSGAPGQRVTISCTGSSSNIGAGYDVHWYQQLPGTAPKLLIYGNSNRPSGVPDR
FSGSKSGTSASLAITGLQAEDEADYYCQSYDSSLRGYVFGTGTKLTVL (SEQ ID NO: 309)
CAGGCTGTGCTGACTCAGCCGTCCTCAGTGTCTGGGGCCCCAGGGCAGAGGGTCACCATC
TCCTGCACTGGGAGCAGCTCCAACATCGGGGCAGGTTATGATGTACACTGGTACCAGCA
GCTTCCAGGAACAGCCCCCAAACTCCTCATCTATGGTAACAGCAATCGGCCCTCAGGGGT
CCCTGACCGATTCTCTGGCTCCAAGTCTGGCACCTCAGCCTCCCTGGCCATCACTGGGCT
CCAGGCTGAGGATGAGGCTGATTATTACTGCCAGTCCTATGACAGCAGCCTGCGTGGTTA
TGTCTTCGGAACTGGGACCAAGCTGACCGTCCTA (SEQ ID NO: 310)
Her2_S1R2A_DP47_5D6
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARGSRVGTIWGSLDDWGKGTMVTVSS (SEQ ID NO: 311)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGG
CAGTAGAGTGGGGACGATTTGGGGAAGCCTTGACGACTGGGGCAAAGGGACAATGGTCA
CCGTCTCGAGT (SEQ ID NO: 312)
V_{L} with CDR1, CDR2 and CDR3 underlined
ETTLTQSPGTLSLSPGERATLSCRASQSSSSSYLAWYQQKPGQAPRLLIYAASSRATGVPDRFS
GSGSGTDFTLTISRLEPEDFAVYYCQQYGSSRFTFGQGTRLEIKR (SEQ ID NO: 313)
GAAACGACACTCACGCAGTCTCCAGGCACCCTGTCTTTGTCTCCAGGGGAAAGAGCCAC
CCTCTCCTGCAGGGCCAGTCAGAGTAGTAGCAGCAGCTACTTAGCCTGGTACCAGCAGA
AACCTGGCCAGGCTCCCAGGCTCCTCATCTATGCTGCATCCAGCAGGGCCACTGGCGTCC
CAGACAGGTTCAGTGGCAGTGGGTCTGGGACAGACTTCACTCTCACCATCAGCAGACTG
GAGCCTGAAGATTTTGCAGTGTATTACTGTCAGCAGTATGGTAGCTCACGGTTCACCTTC
GGCCAAGGGACACGACTGGAGATTAAACGT (SEQ ID NO: 314)
Her2_S1R3A1_DP47_11F6
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCGRGSRVGTIWGSLDFWGQGTMVTVSS (SEQ ID NO: 315)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGGGAGAGG
CAGCAGAGTGGGGACGATTTGGGGAAGCCTTGACTTTTGGGGCCAAGGGACAATGGTCA
CCGTCTCGAGT (SEQ ID NO: 316)
V_{L} with CDR1, CDR2 and CDR3 underlined
QSVLTQPPSVSAAPGQRVTISCSGKSSNIGGNSVAWYQQLPGTAPKVLIYDNDKRPSGVPDRF
SGSKSGTSATLGITGLQTGDEADYYCGSWDSSLGVGMFGGGTKVTVL (SEQ ID NO: 317)
CAGTCTGTGCTGACGCAGCCGCCCTCAGTGTCTGCGGCCCCGGGACAGAGGGTCACCATC
TCCTGCTCTGGAAAGAGCTCCAACATTGGCGGTAATTCTGTGGCCTGGTACCAGCAACTC
CCGGGAACAGCCCCCAAAGTCCTCATTTATGACAATGATAAGCGACCCTCAGGGGTTCCT
GACCGATTCTCTGGCTCCAAGTCTGGCACGTCAGCCACCCTGGGCATCACCGGACTGCAG
ACTGGGGACGAGGCCGATTATTACTGCGGATCCTGGGATAGCAGCCTGGGTGTTGGGAT
GTTCGGCGGAGGGACCAAGGTCACCGTCCTA (SEQ ID NO: 318)
Her2_S1R3A1_DP47_11D3
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARGYSGSSFDAWGQGTMVTVSS (SEQ ID NO: 319)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGGGG
CTACAGTGGAAGTTCCTTTGACGCCTGGGGCCAAGGGACAATGGTCACCGTCTCGAGT (SEQ ID NO: 320)
V_{L} with CDR1, CDR2 and CDR3 underlined
QSVLTQPPSASGTPGQRVTISCSGSSSNIGSKSVYWYQQLPGAAPKLLIYRNSQRPSGVPDRFS
ASKSGTSASLAISGLRSEDEADYYCAAWDGSLSGHFFGTGTKLTVL (SEQ ID NO: 321)
CAGTCTGTGTTGACGCAGCCGCCATCAGCGTCCGGGACCCCCGGGCAGAGGGTCACCAT
CTCTTGTTCTGGAAGCAGCTCCAATATCGGAAGTAAGTCTGTATACTGGTACCAGCAACT
CCCAGGAGCGGCCCCCAAACTCCTCATCTACAGGAATAGTCAGCGGCCCTCAGGGGTCC
CTGACCGATTCTCTGCCTCCAAGTCTGGCACCTCTGCCTCCCTGGCCATCAGTGGGCTCCG
GTCCGAGGATGAGGCTGACTATTACTGTGCAGCATGGGATGGCAGCCTGAGTGGACATT
TCTTCGGAACTGGGACCAAGCTGACCGTCCTA (SEQ ID NO: 322)
Her2_S1R3A1_CS_8A8
V_{H} with CDR1, CDR2 and CDR3 underlined
EVQLVQSGAEVKKPGESLKISCKGSGYSFTSYWIGWVRQMPGKGLEWMGIIYPGDSDTRYSP
SFQGQVTISADKSISTAYLQWSSLKASDTAMFYCARLNDSSGYTTNFDYWGQGTLVTVSS (SEQ ID NO: 323)
GAGGTGCAGCTGGTGCAGTCTGGGGCAGAGGTGAAAAAGCCCGGGGAGTCTCTGAAGAT
CTCCTGTAAGGGCTCTGGATACAGCTTTACCAGCTATTGGATCGGCTGGGTGCGCCAGAT
GCCCGGGAAAGGCCTGGAGTGGATGGGGATCATCTATCCTGGTGACTCTGATACCAGAT
ACAGCCCGTCCTTCCAAGGCCAGGTCACCATCTCAGCCGACAAGTCCATCAGCACCGCCT
ACCTGCAGTGGAGCAGCCTGAAGGCCTCGGACACCGCCATGTTTTACTGTGCGAGACTCA
ATGATAGTAGTGGTTATACGACTAACTTTGACTACTGGGGCCAAGGCACCCTGGTCACCG
TCTCGAGT (SEQ ID NO: 324)
V_{L} with CDR1, CDR2 and CDR3 underlined
QAVVIQEPSFSVSPGGTVTLTCGLSSGSVSTRYNPSWYQQTPGQAPRTLIYSTNTRSSGVPDRF
SGSILGNKAALTITGAQADDESDYYCALYMGSGIWVFGGGTKLTVL (SEQ ID NO: 325)
CAGGCTGTGGTGATCCAGGAGCCATCGTTCTCAGTGTCCCCTGGAGGGACAGTCACACTC
ACTTGTGGCTTGAGCTCTGGCTCAGTCTCTACTCGTTACAACCCCAGCTGGTACCAGCAG
ACCCCAGGCCAGGCTCCACGCACGCTCATCTACAGTACAAACACTCGTTCTTCTGGGGTC
CCTGACCGCTTCTCTGGCTCCATCCTTGGGAACAAAGCTGCCCTCACCATCACGGGGGCC
CAGGCAGATGATGAATCTGATTATTACTGTGCGCTGTATATGGGTAGTGGCATTTGGGTG
TTCGGCGGAGGGACCAAGCTGACCGTCCTA (SEQ ID NO: 326)
Her2_S1R3A1_BMV_5D10
V_{H} with CDR1, CDR2 and CDR3 underlined
EVQLVESGGGLVQPGGSLRLSCAASGFTFDSYAMSWVRQAPGKGLEWVSIISGRDGYTYYT
DSVKGRFTISRDNSKNTVYLQMNSLRAEDTGVYYCARNGEWPGILDYWGRGTMVTVSS (SEQ ID NO: 327)
GAGGTGCAGCTGGTGGAGTCTGGGGGAGGCTTGGTACAGCCTGGGGGGTCCCTGAGACT
CTCCTGTGCTGCCTCTGGATTCACCTTTGACAGCTATGCCATGAGCTGGGTCCGCCAGGC
TCCAGGGAAGGGGCTGGAGTGGGTCTCAATTATTAGTGGTAGAGATGGTTACACATACT
ACACAGACTCCGTGAAGGGTCGGTTCACCATCTCCAGAGACAATTCCAAGAACACGGTG
TATCTGCAAATGAACAGCCTGAGAGCCGAGGACACGGGTGTGTATTATTGTGCGAGAAA
TGGGGAGTGGCCCGGAATCTTAGACTACTGGGGCAGGGGGACAATGGTCACCGTCTCCT
CA (SEQ ID NO: 328)
V_{L} with CDR1, CDR2 and CDR3 underlined
DIQMTQSPSTLSASIGDRVTITCRASEGIYHWLAWYQQKPGKAPKLLIYKASSLASGAPSRFS
GSGSGTDFTLTISSLQPDDFATYYCQQYSNYPLTFGGGTKLEIKR (SEQ ID NO: 329)
GACATCCAGATGACCCAGTCTCCTTCCACCCTGTCTGCATCTATTGGAGACAGAGTCACC
ATCACCTGCCGGGCCAGTGAGGGTATTTATCACTGGTTGGCCTGGTATCAGCAGAAGCCA
GGGAAAGCCCCTAAACTCCTGATCTATAAGGCCTCTAGTTTAGCCAGTGGGGCCCCATCA
AGGTTCAGCGGCAGTGGATCTGGGACAGATTTCACTCTCACCATCAGCAGCCTGCAGCCT
GATGATTTTGCAACTTATTACTGCCAACAATATAGTAATTATCCGCTCACTTTCGGCGGA
GGGACCAAGCTGGAGATCAAACGT (SEQ ID NO: 330)
Her2_S1R3A1_DP47_11C1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAASNSYYYFDYWGQGTLVTVSS (SEQ ID NO: 331)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGGCGAG
TAATAGTTATTACTACTTTGACTACTGGGGCCAGGGAACCCTGGTCACCGTCTCGAGT (SEQ ID NO: 332)
V_{L} with CDR1, CDR2 and CDR3 underlined
SSELTQDPAVSVALGQTVRITCQGDSLRNFYPSWYQQKPGQAPVLVIYGKNIRPSGIPDRFSG
SGSGSTASLTITGAQAEDEADYYCNSRDSSGKHMGVVFGGGTKLTVL (SEQ ID NO: 333)
TCTTCTGAGCTGACTCAGGACCCTGCTGTGTCTGTGGCCTTGGGACAGACAGTCAGGATC
ACATGCCAAGGAGACAGCCTCAGAAACTTTTATCCAAGTTGGTATCAGCAGAAGCCAGG
ACAGGCCCCTGTTCTTGTCATTTATGGTAAAAATATTCGGCCCTCAGGGATCCCAGACCG
ATTCTCTGGCTCCGGCTCAGGAAGCACAGCTTCCTTGACCATCACTGGGGCTCAGGCGGA
AGATGAGGCTGACTATTACTGTAACTCCCGGGACAGCAGTGGTAAACATATGGGGGTGG
TATTCGGCGGAGGGACCAAGCTGACCGTCCTA (SEQ ID NO: 334)
Her2_S1R3A1_DP47_4E1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDMAVYYCARTPGYSSGWYSVWGRGTLVTVSS (SEQ ID NO: 335)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACATGGCCGTGTATTACTGTGCGAGGAC
TCCCGGGTATAGCAGTGGCTGGTACTCGGTTTGGGGCCGGGGCACCCTGGTCACCGTCTC
GAGT (SEQ ID NO: 336)
V_{L} with CDR1, CDR2 and CDR3 underlined
QSVVTQPPSVSGAPGQRVTISCTGSSSNIGAGYDVHWYQQVPGTAPKLLIYGNNNRPSGVPD
RFSGSKSGTSASLAITGLQPEDEVDYYCQSYDRSLSGYIFGSGTKVTVL (SEQ ID NO: 337)
CAGTCTGTCGTGACGCAGCCGCCCTCAGTGTCTGGGGCCCCAGGGCAGAGGGTCACCAT
CTCCTGTACTGGGAGCAGCTCCAACATCGGGGCAGGGTATGATGTTCACTGGTACCAGCA
GGTTCCAGGAACAGCCCCCAAACTCCTCATCTATGGTAACAACAATCGGCCCTCGGGGGT
CCCTGACCGATTCTCTGGCTCCAAGTCTGGCACCTCAGCCTCCCTGGCCATCACTGGGCT
CCAGCCTGAGGATGAAGTTGATTATTACTGCCAGTCCTATGACCGCAGCCTGAGTGGTTA
TATCTTCGGAAGTGGGACCAAGGTCACCGTCCTA (SEQ ID NO: 338)
Her2_S1R3A1_DP47_10E1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARVGSFGDYKDKSGYGFYFDYWGQG
TLVTVSS (SEQ ID NO: 339)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGT
TGGGTCGTTTGGTGATTACAAAGATAAAAGTGGTTACGGCTTCTACTTTGACTACTGGGG
CCAAGGCACCCTGGTCACCGTCTCGAGT (SEQ ID NO: 340)
V_{L} with CDR1, CDR2 and CDR3 underlined
QSVLTQPPSASGTLGQTVFISCSGSSSNIGSNSVSWYQQLPGTAPKFLIYHNNQRPSGVPERFS
GSKSGTSASLAIRGLQSEDEADYYCASWEDSLNGWVFGGGTKLTVL (SEQ ID NO: 341)
CAGTCTGTGCTGACTCAGCCACCCTCGGCGTCTGGGACCCTCGGGCAGACGGTCTTCATC
TCTTGTTCTGGAAGCAGTTCCAACATCGGAAGTAATTCTGTGAGTTGGTACCAGCAGCTC
CCAGGAACGGCCCCCAAATTTCTCATTTATCATAATAATCAGCGGCCCTCAGGGGTCCCT
GAGCGATTCTCTGGCTCCAAGTCTGGCACCTCAGCCTCCCTGGCCATCCGTGGGCTCCAG
TCTGAGGATGAGGCTGATTACTACTGTGCATCTTGGGAGGACAGCCTGAATGGTTGGGTG
TTCGGCGGGGGGACCAAGCTGACCGTCCTA (SEQ ID NO: 342)
Her2_S1R3A1_CS_11C3
V_{H} with CDR1, CDR2 and CDR3 underlined
QVQLVQSGAEVKKPGASVKVSCKASGYTFTSYGISWVRQAPGQGLEWMGWISAYNGNTNY
AQKLQGRVTMTFTTDTSTSTAYMELRSLRSDDTAVYYCARVGSGYCSGGSCYVGWFDPWGRG
TMVTVSS (SEQ ID NO: 343)
CAGGTCCAGCTGGTGCAGTCTGGGGCTGAGGTGAAGAAGCCTGGGGCCTCAGTGAAGGT
CTCCTGCAAGGCTTCTGGTTACACCTTTACCAGCTATGGTATCAGCTGGGTGCGACAGGC
CCCTGGACAAGGGCTTGAGTGGATGGGATGGATCAGCGCTTACAATGGTAACACAAACT
ATGCACAGAAGCTCCAGGGCAGAGTCACCATGACCACAGACACATCCACGAGCACAGCC
TACATGGAGCTGAGGAGCCTGAGATCTGACGACACGGCCGTGTATTACTGTGCGAGAGT
GGGGTCGGGATATTGTAGTGGTGGTAGCTGCTACGTGGGCTGGTTCGACCCCTGGGGCCG
GGGGACAATGGTCACCGTCTCGAGT (SEQ ID NO: 344)
V_{L} with CDR1, CDR2 and CDR3 underlined
SSELTQDPAVSVALGQTVKITCQGDSLSAYYATWYQQKPGQAPVLVIYGKNKRPSGIPDRFS
GSKSGNTASLTITGAQAEDEADYYCNSRDSSGNDHYVFGTGTKLTVL (SEQ ID NO: 345)
TCTTCTGAGCTGACTCAGGACCCTGCTGTGTCTGTGGCCTTGGGACAGACAGTCAAGATC
ACATGCCAAGGAGACAGCCTCAGTGCCTATTATGCAACCTGGTACCAGCAGAAGCCAGG
CCAGGCCCCTGTACTTGTCATCTATGGTAAAAACAAGCGGCCGTCCGGGATCCCAGACCG
ATTCTCTGGCTCCAAGTCAGGAAACACAGCTTCCTTGACCATCACGGGGGCTCAGGCGGA
AGATGAGGCTGACTATTACTGTAACTCCCGGGACAGCAGTGGTAATGATCATTATGTCTT
CGGAACTGGGACCAAGCTGACCGTTCTA (SEQ ID NO: 346)
Her2_S1R3A1_CS_13H11
V_{H} with CDR1, CDR2 and CDR3 underlined
EVQLVQSGAEVRKPGASVKVSCRASGYNFKDYYLHWVRQAPGEGLEWMGWINPHAGTTK
YAQNFQHRIIMTRDTTITTAYMELSSLKSDDTAIYFCTRYYFDSSGYFRFDPWGQGTMVTVSS (SEQ ID NO: 347)
GAGGTCCAGCTGGTACAGTCTGGGGCTGAGGTGAGGAAGCCTGGGGCCTCAGTCAAGGT
CTCCTGCAGGGCTTCTGGATACAACTTCAAAGACTACTATTTGCACTGGGTGCGCCAGGC
CCCTGGAGAAGGGCTTGAGTGGATGGGGTGGATCAACCCTCACGCTGGTACCACAAAAT
ATGCACAGAATTTTCAGCACAGGATTATTATGACCAGGGACACGACCATCACCACAGCC
TACATGGAACTGAGCAGTCTGAAATCTGACGACACAGCCATTTATTTCTGTACCAGATAC
TACTTTGACAGTAGTGGTTATTTTAGGTTCGACCCCTGGGGCCAAGGGACAATGGTCACC
GTCTCGAGT (SEQ ID NO: 348)
V_{L} with CDR1, CDR2 and CDR3 underlined
QSVVTQPPSVSGAPGQKVTISCSGSSSNIGNNYVSWYQQLPGTAPKLLIYDNNKRPSGIPDRFS
GSKSGTSATLGITGLQTGDEADYYCGTWDSSLSAGVFGGGTKVTVL (SEQ ID NO: 349)
CAGTCTGTCGTGACGCAGCCGCCCTCAGTGTCTGGGGCCCCAGGACAGAAGGTCACCAT
CTCCTGCTCTGGAAGCAGCTCCAACATTGGGAATAATTATGTATCCTGGTACCAGCAGCT
CCCAGGAACAGCCCCCAAACTCCTCATTTATGACAATAATAAGCGACCCTCAGGGATTCC
TGACCGATTCTCTGGCTCCAAGTCTGGCACGTCAGCCACCCTGGGCATCACCGGACTCCA
GACTGGGGACGAGGCCGATTATTACTGCGGAACATGGGATAGCAGCCTGAGTGCTGGGG
TGTTCGGCGGAGGGACCAAGGTCACCGTCCTA (SEQ ID NO: 350)
Her2_S1R3A1_CS_2D9
V_{H} with CDR1, CDR2 and CDR3 underlined
QMQLVQSGAEVKKPGESLKMSCKGSGYSFTSYWIGWVRQMPGKGLEWMGIIYPGNSDTRY
NPSFEGQVTISADKSINTAFLQWNSLKASDTAIYYCARAPWVGAFDTWGQGTMVTVSS (SEQ ID NO: 351)
CAGATGCAGCTGGTGCAGTCTGGGGCAGAGGTGAAAAAGCCCGGGGAGTCTCTGAAGAT
GTCCTGTAAGGGTTCTGGATACAGCTTTACCAGCTACTGGATCGGCTGGGTGCGCCAGAT
GCCCGGGAAAGGCCTGGAGTGGATGGGGATCATCTATCCTGGTAACTCCGATACCAGAT
ACAACCCGTCCTTCGAAGGCCAGGTCACCATCTCAGCCGACAAGTCCATCAACACCGCCT
TCCTGCAGTGGAACAGCCTGAAGGCCTCGGACACCGCCATATATTATTGTGCGCGGGCTC
CCTGGGTGGGTGCTTTTGATACTTGGGGCCAGGGGACAATGGTCACCGTCTCTTCA (SEQ ID NO: 352)
V_{L} with CDR1, CDR2 and CDR3 underlined
DIVMTQSPSTLSASVGDRVTITCRASQGISSWLAWYQQKPGRAPKVLIYKASTLESGVPSRFS
GSGSGTDFTLTISSLQPEDFATYYCQQSYSTPWTFGQGTKLEIKR (SEQ ID NO: 353)
GACATCGTGATGACCCAGTCTCCTTCCACCCTGTCTGCATCTGTAGGAGACAGAGTCACC
ATCACTTGCCGGGCCAGTCAGGGTATTAGTAGCTGGTTGGCCTGGTATCAGCAGAAACCA
GGGAGAGCCCCTAAGGTCTTGATCTATAAGGCATCTACTTTAGAAAGTGGGGTCCCATCA
AGGTTCAGCGGCAGTGGATCTGGGACAGATTTCACTCTCACCATCAGCAGTCTGCAACCT
GAAGATTTTGCAACTTACTACTGTCAACAGAGTTACAGTACCCCGTGGACGTTCGGCCAA
GGGACCAAGCTGGAGATCAAACGT (SEQ ID NO: 354)
Her2_S1R2A_CS_3D4
V_{H} with CDR1, CDR2 and CDR3 underlined
QMQLVQSGAEVKKPGASVKVSCKSSGYTFKDYYINWVRQAPGQGLEWVGWINPKNGITKY
SQNFQGRVSMTTDTSISTVYMDLRGLTSDDTAVYYCARDANRLRVGWFDPWGQGTLVTVSS (SEQ ID NO: 355)
CAGATGCAGCTGGTGCAGTCTGGGGCTGAGGTGAAGAAGCCAGGGGCCTCAGTGAAAGT
CTCCTGCAAGTCTTCTGGATACACCTTCAAGGACTACTATATCAACTGGGTGCGACAGGC
CCCTGGACAAGGGCTTGAGTGGGTGGGATGGATCAACCCTAAAAATGGTATCACAAAAT
ATTCGCAGAATTTTCAGGGCAGGGTCTCCATGACCACGGATACGTCCATCAGCACAGTCT
ACATGGACCTGAGAGGTCTGACATCTGACGACACGGCCGTTTATTATTGTGCGAGAGAC
GCGAACCGCCTTAGGGTGGGCTGGTTCGACCCCTGGGGCCAAGGAACCCTGGTCACCGT
CTCGAGT (SEQ ID NO: 356)
V_{L} with CDR1, CDR2 and CDR3 underlined
QAVLTQPSSVSGSPGQRVSISCTGSSSNIGAGYDVHWYQQLPGTAPKLLIYGNINRPSGVPDR
FSGSKSGTSASLAITGLQAEDEADYYCQSYDSSLRAAVFGGGTKVTVL (SEQ ID NO: 357)
CAGGCTGTGCTGACTCAGCCGTCCTCAGTGTCTGGGTCCCCAGGGCAGAGGGTCAGCATC
TCCTGCACTGGGAGCAGCTCCAACATCGGGGCAGGTTATGATGTACATTGGTATCAACAA
CTTCCAGGAACAGCCCCCAAACTCCTCATCTACGGTAACATCAATCGGCCCTCAGGGGTC
CCTGACCGATTCTCTGGCTCCAAGTCTGGCACCTCAGCCTCCCTGGCCATCACTGGGCTC
CAGGCTGAAGATGAGGCTGATTATTACTGCCAGTCCTATGACAGCAGCCTGAGGGCTGC
GGTATTCGGCGGAGGGACCAAGGTCACCGTCCTA (SEQ ID NO: 358)
Her2_S1R3A1_DP47_2H6
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKDFWFGLPPSFFDSWGQGTMVTVSS (SEQ ID NO: 359)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAAAGA
TTTCTGGTTTGGACTACCACCTTCCTTCTTTGACTCTTGGGGCCAAGGGACAATGGTCACC
GTCTCGAGT (SEQ ID NO: 360)
V_{L} with CDR1, CDR2 and CDR3 underlined
QSVLTQPPSVSVSPGQKASITCSGERMGDKYAAWYQQKPGQSPILVIYQDTKRPSGIPERFSG
SNSGNTATLTISGTQDMDEADYYCQVWDSSTGVFGGGTKVTVL (SEQ ID NO: 361)
CAGTCTGTGCTGACTCAGCCACCCTCAGTGTCCGTGTCCCCAGGACAGAAGGCCAGCATC
ACCTGCTCTGGAGAAAGAATGGGGGATAAATATGCTGCCTGGTATCAGCAGAAGCCAGG
CCAGTCACCTATACTGGTCATCTATCAAGATACAAAGCGGCCCTCAGGGATCCCTGAGCG
ATTCTCTGGCTCCAACTCTGGGAACACAGCCACGTTGACCATCAGCGGGACCCAGGACAT
GGATGAGGCTGACTATTACTGTCAGGTGTGGGACAGCAGCACTGGGGTATTCGGCGGAG
GGACCAAGGTCACCGTCCTA (SEQ ID NO: 362)
Her2_S1R3A1_DP47_4G1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKDLNPYSVVTLGMDVWGRGTMVTV
SS (SEQ ID NO: 363)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAAAGA
TCTGAACCCTTATTCAGTGGTAACTCTCGGTATGGACGTCTGGGGCAGAGGGACAATGGT
CACCGTCTCGAGT (SEQ ID NO: 364)
V_{L} with CDR1, CDR2 and CDR3 underlined
QSVLTQPPSVSVAPGQAARIPCGGDNIGSKSVHWYQQRPGQAPVLVVFDDSDRPSGIPERFSG
SNSGHTATLTINRVEPGDEAEYYCEVWDGGERHVVFGGGTKLTVL (SEQ ID NO: 365)
CAGTCTGTGCTGACTCAGCCACCCTCGGTGTCAGTGGCCCCAGGACAGGCGGCCAGGATT
CCCTGTGGGGGAGACAACATTGGAAGTAAGAGTGTTCACTGGTACCAGCAGAGGCCAGG
CCAGGCCCCTGTCCTGGTCGTCTTTGATGATAGTGACCGGCCCTCAGGGATCCCTGAGCG
ATTCTCTGGCTCCAATTCTGGGCACACGGCCACCCTGACCATCAACAGGGTCGAACCCGG
GGATGAGGCCGAGTATTATTGTGAGGTGTGGGATGGTGGCGAGAGACATGTGGTATTCG
GCGGAGGGACCAAGCTGACCGTCCTA (SEQ ID NO: 366)
Her2_S1R2A_DP47_3C1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARDLTGPNHWFFDLWGRGTTVTVSS (SEQ ID NO: 367)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCCAGAGA
TTTAACTGGCCCAAACCACTGGTTCTTCGATCTCTGGGGGCGGGGGACCACGGTCACCGT
CTCGAGT (SEQ ID NO: 368)
V_{L} with CDR1, CDR2 and CDR3 underlined
QSVLTQPPSVSVSPGQTASISCSGHRLGDKYVSWYRQRPGQSPVLVIYQDEKRYSGISERFSGS
NSGNVATLTITGTQAMDEADYHC QAWDSTTVVFGGGTKLTVL (SEQ ID NO: 369)
CAGTCTGTGCTGACTCAGCCACCCTCCGTGTCCGTGTCCCCAGGACAGACAGCCAGCATC
TCCTGCTCTGGACATAGATTGGGCGATAAGTATGTTAGTTGGTATCGGCAGAGGCCGGGC
CAGTCCCCTGTGCTGGTCATCTATCAAGATGAGAAGAGGTACTCAGGGATCTCTGAGCGA
TTCTCTGGCTCCAACTCTGGGAACGTAGCCACTCTGACCATCACCGGGACCCAGGCTATG
GATGAGGCTGACTATCACTGTCAGGCGTGGGACAGCACCACTGTGGTGTTCGGCGGAGG
GACCAAGCTGACCGTCCTA (SEQ ID NO: 370)
Her2_S1R3A1_DP47_7B2
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARDRRPRDDAFDMWGRGTTVTVSS (SEQ ID NO: 371)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGA
TAGACGGCCGAGGGATGATGCTTTTGATATGTGGGGGAGAGGGACCACGGTCACCGTCT
CGAGT (SEQ ID NO: 372)
V_{L} with CDR1, CDR2 and CDR3 underlined
DIQMTQSPSSVSASVGDRVSITCRASQGIGSWLFWYQQKPGKAPILLMSAVSGLQSGVPSRFS
GSGSGTDFTLTISSVQPEDFATYYCQQAHSFPITFGQGTRLEIKR (SEQ ID NO: 373)
GACATCCAGATGACCCAGTCTCCCTCTTCTGTGTCTGCTTCTGTTGGAGACAGAGTCAGC
ATCACTTGTCGGGCGAGTCAGGGAATTGGCAGCTGGTTATTCTGGTATCAGCAGAAACCA
GGGAAAGCCCCTATCCTCCTGATGTCCGCTGTGTCCGGTTTGCAAAGTGGAGTCCCATCA
CGATTCAGCGGCAGCGGATCTGGGACAGATTTCACTCTCACGATCAGCAGCGTACAGCCT
GAGGATTTTGCAACTTACTATTGTCAACAGGCTCACAGTTTCCCTATCACCTTCGGCCAA
GGGACACGACTGGAGATTAAACGT (SEQ ID NO: 374)
Her2_S1R3B2_DP47_4E2
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARDRVSNWNYYGQDSYFDYWGQGT
MVTVSS (SEQ ID NO: 375)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGGGA
CAGGGTCTCTAACTGGAACTACTACGGCCAGGACAGCTACTTTGACTACTGGGGCCAGG
GGACAATGGTCACCGTCTCGAGT (SEQ ID NO: 376)
V_{L} with CDR1, CDR2 and CDR3 underlined
QSVLTQPPSASGAPGQRVTISCSGTNSNIGSNNVNWYQQLPGKAPRLLIYNNNQRPSGVPDRF
SGSKSGTSASLAISGLQSELEADYYCSAWDDSLHGPVFGGGTKLTVL (SEQ ID NO: 377)
CAGTCTGTGCTGACTCAGCCACCCTCCGCGTCTGGGGCCCCCGGGCAGAGGGTCACCATT
TCTTGTTCTGGGACCAACTCCAACATCGGAAGTAATAATGTAAACTGGTATCAGCAACTC
CCAGGAAAGGCCCCCAGACTCCTCATCTACAATAATAATCAGAGGCCCTCAGGGGTCCC
TGACCGATTCTCTGGCTCCAAGTCTGGCACCTCAGCCTCCCTGGCCATCAGTGGGCTCCA
GTCTGAGCTTGAGGCTGATTATTATTGTTCAGCATGGGATGACAGCCTGCATGGTCCGGT
GTTCGGCGGAGGGACCAAGCTGACCGTCCTA (SEQ ID NO: 378)
Her2_S1R3A1_CS_16C2
V_{H} with CDR1, CDR2 and CDR3 underlined
EVQLVESGGGLAQPGGSLRLSCAASGLTFTTYAMSWVRQAPGKGLEWVSSISGSGHSTYYA
DSVKGRFTISRDISKNTLYLQMNSLRAEDTAVYYCAKDSSAFGFVHGAFDIWGQGTLVTVSS (SEQ ID NO: 379)
GAGGTGCAGCTGGTGGAGTCTGGGGGAGGCTTGGCACAGCCTGGGGGGTCCCTGAGACT
CTCCTGTGCAGCCTCTGGATTAACCTTTACCACCTATGCCATGAGCTGGGTCCGCCAGGC
TCCAGGGAAGGGGCTGGAGTGGGTCTCAAGTATTAGTGGAAGTGGTCATAGCACATATT
ACGCAGACTCCGTGAAGGGCCGCTTCACCATCTCCAGAGACATTTCCAAGAACACGTTGT
ATCTGCAAATGAACAGCCTCAGAGCCGAGGACACGGCCGTCTATTACTGTGCGAAAGAT
TCGTCGGCTTTTGGGTTTGTACACGGTGCTTTTGATATCTGGGGCCAGGGAACCCTGGTC
ACCGTCTCGAGT (SEQ ID NO: 380)
V_{L} with CDR1, CDR2 and CDR3 underlined
SSELTQDPAASVALGQTVSITCQGDSLRNYWASWYQQKPGQAPVLVIYGKNTRPSGIPDRFS
GSTSGNTASLTITGAQAEDEADYYCNSRDSGHRLLFGGGTKLTVL (SEQ ID NO: 381)
TCTTCTGAGCTGACTCAGGACCCTGCTGCGTCTGTGGCCTTGGGACAGACAGTCAGCATC
ACATGCCAAGGAGACAGCCTCAGAAACTATTGGGCTAGCTGGTACCAGCAGAAGCCAGG
ACAGGCCCCTGTACTTGTCATCTATGGTAAAAATACCCGGCCCTCAGGGATCCCAGACCG
ATTCTCTGGCTCCACCTCAGGAAACACAGCTTCCTTGACCATCACTGGGGCTCAGGCGGA
GGATGAGGCTGACTATTACTGCAACTCCCGGGACAGTGGTCACCGTCTTCTTTTCGGCGG
AGGGACCAAGCTGACCGTCCTA (SEQ ID NO: 382)
Her2_S1R3A1_CS_11E5
V_{H} with CDR1, CDR2 and CDR3 underlined
EVQLVQSGAEVKRPGESLKISCRASGYIFTNNWVAWVRQQPGKGLEWMGIIYPGDSDTRYSP
SFQGQVTFSADTSINTAYLQWNSLKASDTATYFCAREAYNSYEYYGMDVWGRGTTVTVSS (SEQ ID NO: 383)
GAGGTGCAGCTGGTGCAGTCTGGGGCAGAAGTCAAGAGGCCCGGAGAGTCTCTGAAGAT
CTCCTGTAGGGCCTCTGGATACATCTTTACGAACAATTGGGTCGCCTGGGTGCGCCAGCA
GCCCGGGAAAGGCCTGGAGTGGATGGGGATCATCTATCCTGGTGACTCTGACACCAGAT
ACAGCCCGTCCTTCCAAGGCCAGGTCACTTTCTCGGCCGACACGTCCATCAACACCGCCT
ACCTACAGTGGAATAGCCTGAAGGCCTCGGACACCGCCACTTACTTCTGTGCGCGAGAG
GCCTACAACTCATACGAATATTACGGTATGGACGTCTGGGGGCGAGGGACCACGGTCAC
CGTCTCGAGT (SEQ ID NO: 384)
V_{L} with CDR1, CDR2 and CDR3 underlined
QTVVIQEPSFSVSPGGTVTLTCGLSSGSVSTNYYPSWYQQTPGQAPRTLIYNTNTRSSGVPDRF
SGSILGNKAALTITGAQADDESDYYCVLYMGSGISVFGGGTKVTVL (SEQ ID NO: 385)
CAGACTGTGGTGATCCAGGAGCCATCGTTCTCAGTGTCCCCTGGAGGGACAGTCACACTC
ACTTGTGGCTTGAGCTCTGGCTCAGTCTCTACTAATTACTACCCCAGCTGGTACCAGCAG
ACCCCAGGCCAGGCTCCACGCACGCTCATCTACAACACAAACACTCGCTCTTCTGGGGTC
CCTGATCGCTTCTCTGGCTCCATCCTTGGGAACAAAGCTGCCCTCACCATCACGGGGGCC
CAGGCAGATGATGAATCTGATTATTACTGTGTGCTGTATATGGGTAGTGGCATTTCGGTG
TTCGGCGGAGGGACCAAGGTCACCGTCCTA (SEQ ID NO: 386)
Her2_S1R3A1_CS_16D7
V_{H} with CDR1, CDR2 and CDR3 underlined
EVQLVESGAEVKKPGESLKISCKASGYIFTNNWIAWVRQQPGKGLEWMGIIYPGDSDTRYSP
SFQGRVTFSADTSINTAYLQWSSLKASDTATYYCAREAYNSYEYYGMDVWGQGTMVTVSS (SEQ ID NO: 387)
GAGGTGCAGCTGGTGGAGTCCGGAGCAGAAGTCAAAAAGCCCGGAGAGTCTCTGAAGAT
CTCCTGTAAGGCTTCTGGATACATCTTTACGAACAATTGGATCGCCTGGGTGCGGCAGCA
GCCCGGGAAAGGCCTGGAGTGGATGGGAATCATCTATCCTGGTGACTCTGACACCAGAT
ACAGCCCGTCCTTCCAGGGCCGGGTCACTTTCTCAGCCGACACGTCCATCAACACCGCCT
ACCTCCAGTGGAGTAGCCTGAAGGCCTCGGACACCGCCACTTACTACTGTGCGAGAGAG
GCCTACAACTCATACGAGTACTACGGTATGGACGTCTGGGGCCAAGGGACAATGGTCAC
CGTCTCGAGT (SEQ ID NO: 388)
V_{L} with CDR1, CDR2 and CDR3 underlined
QTVVLQEPAFSVSPGGTVTLTCGLSSGSVSTSYYPSWYQQTPGQPPRTLIYNTNTRSSGVSDR
FSGSILGNKAALTITGAQAEDESDYYCVLYMGSGISVFGGGTKLTVL (SEQ ID NO: 389)
CAGACTGTGGTGCTCCAGGAGCCAGCGTTCTCAGTGTCCCCTGGAGGGACAGTCACACTC
ACCTGTGGCTTGAGCTCTGGCTCAGTCTCTACTAGTTACTACCCCAGTTGGTACCAGCAG
ACCCCAGGCCAGCCTCCACGCACGCTCATCTACAACACAAACACCCGCTCTTCTGGGGTC
TCTGATCGCTTCTCTGGCTCCATCCTTGGGAACAAAGCTGCCCTCACCATCACGGGGGCC
CAGGCCGAAGATGAATCTGATTATTACTGTGTTCTGTATATGGGTAGTGGCATTTCGGTA
TTCGGCGGGGGGACCAAGCTGACCGTCCTA (SEQ ID NO: 390)
Her2_S1R2A_CS_10B10
V_{H} with CDR1, CDR2 and CDR3 underlined
EVQLVQSGAEVKKPGASVRVSCKGSGNTFTGHYIHWVRQAPGQGLEWLGWIDPNTGDIQYS
ENFKGSVTLTRDPSINSVFMDLIRLTSDDTAMYYCAREGAGLANYYYYGLDVWGRGTMVT
VSS (SEQ ID NO: 391)
GAAGTGCAGCTGGTGCAGTCTGGGGCTGAGGTGAAGAAGCCTGGGGCCTCAGTGAGGGT
CTCCTGCAAGGGTTCTGGAAACACCTTCACCGGCCACTACATCCACTGGGTGCGACAGGC
CCCTGGACAAGGACTTGAGTGGCTGGGATGGATCGACCCTAACACTGGTGACATACAGT
ATTCAGAAAACTTTAAGGGCTCGGTCACCTTGACCAGGGACCCATCCATCAACTCAGTCT
TCATGGACCTGATCAGGCTGACATCTGACGACACGGCCATGTATTACTGTGCGAGAGAA
GGTGCCGGGCTCGCCAACTACTATTACTACGGTCTGGACGTCTGGGGCCGAGGGACAAT
GGTCACCGTCTCGAGT (SEQ ID NO: 392)
V_{L} with CDR1, CDR2 and CDR3 underlined
QTVVLQEPSFSVSPGGTVTLTCGLNFGSVSTAYYPSWYQQTPGQAPRTLIYGTNIRSSGVPDR
FSGSIVGNKAALTITGAQTEDESDYYCALYMGSGMLFGGGTKVTVL (SEQ ID NO: 393)
CAGACTGTGGTGCTCCAGGAGCCTTCGTTCTCAGTGTCCCCTGGGGGGACAGTCACACTC
ACTTGTGGCTTGAACTTTGGCTCAGTCTCTACTGCTTACTACCCCAGTTGGTACCAGCAGA
CCCCAGGCCAAGCTCCACGCACGCTCATCTACGGCACAAATATTCGTTCCTCTGGGGTCC
CGGATCGCTTCTCTGGCTCCATCGTAGGGAACAAAGCTGCCCTCACCATCACGGGGGCCC
AGACAGAAGATGAGTCTGATTATTATTGTGCGCTGTATATGGGTAGTGGCATGCTCTTCG
GCGGCGGGACCAAGGTCACCGTCCTA (SEQ ID NO: 394)
Her2_S1R3A1_CS_15C2
V_{H} with CDR1, CDR2 and CDR3 underlined
QVQLQESGPGLVKPAGTLSLTCAVSGDSISSNHWWNWVRQSPGKGLEWIGEIFHSDIRILNPS
LKRRVSMSVDRSKDQFSLQLTSVTAADTAVYYCARGFHGDSGRGLDTWGRGTLVTVSS (SEQ ID NO: 395)
CAGGTGCAGCTGCAGGAGTCCGGCCCAGGACTCGTGAAGCCTGCGGGGACTCTGTCCCT
CACCTGCGCTGTCTCCGGTGACTCCATCAGCAGCAATCACTGGTGGAATTGGGTCCGCCA
GTCCCCAGGGAAGGGACTGGAATGGATTGGTGAAATCTTTCATAGTGACATTCGCATCCT
CAACCCGTCCCTCAAGAGGCGCGTCTCCATGTCAGTCGACAGGTCCAAGGACCAATTCTC
CCTGCAACTGACCTCTGTGACCGCCGCGGACACGGCCGTGTATTACTGTGCGAGAGGTTT
CCATGGTGACTCCGGCAGAGGACTTGACACCTGGGGCAGAGGAACCCTGGTCACCGTCT
CGAGT (SEQ ID NO: 396)
V_{L} with CDR1, CDR2 and CDR3 underlined
SSELTQDPAVSVALGQTVRVTCQGDGLRSYYASWYQQKPGQAPVLVMYGNNNRPSGIPDRF
SGSSSGNTASLTITGAQAEDEAVYYCNSRDSGANHLEVFGGGTKVTVL (SEQ ID NO: 397)
TCTTCTGAGCTGACTCAGGACCCTGCTGTGTCTGTGGCCTTGGGACAGACAGTCAGGGTC
ACATGCCAAGGAGACGGCCTCAGAAGTTATTATGCAAGCTGGTACCAGCAGAAGCCAGG
GCAGGCCCCTGTCCTTGTCATGTATGGGAACAACAACCGGCCCTCAGGGATCCCAGACC
GATTCTCTGGCTCCAGCTCGGGAAACACAGCTTCCTTGACCATCACTGGGGCTCAGGCGG
AAGATGAGGCTGTCTATTATTGTAATTCGCGGGACAGCGGTGCTAACCATCTGGAGGTTT
TCGGCGGAGGGACCAAGGTCACCGTCCTA (SEQ ID NO: 398)
Her2_S1R3A1_CS_9C1
V_{H} with CDR1, CDR2 and CDR3 underlined
QVQLQESGPGLVKPSETLSLTCTVSGYSISSGYYWGWIRQPPGRGLEWIGTIYHSGSTYYNPS
LKSRLTISVDTSENQFSLKLSSVTAADTAVYYCARGIAGRTHYDYWGQGTMVTVSS (SEQ ID NO: 399)
CAGGTGCAGCTGCAGGAGTCGGGCCCAGGACTGGTGAAGCCTTCGGAGACCCTGTCCCT
CACCTGCACTGTCTCTGGTTACTCCATTAGCAGTGGTTACTACTGGGGCTGGATCCGGCA
GCCCCCAGGGAGGGGGCTGGAGTGGATTGGGACTATCTATCATAGTGGGAGCACCTACT
ACAACCCGTCCCTCAAGAGTCGACTCACCATATCAGTAGACACGTCCGAGAACCAATTCT
CCCTGAAGCTGAGTTCTGTGACCGCCGCAGACACGGCCGTGTATTACTGTGCGAGAGGG
ATAGCAGGTCGGACCCATTATGACTACTGGGGCCAGGGGACAATGGTCACCGTCTCGAGT (SEQ ID NO: 400)
V_{L} with CDR1, CDR2 and CDR3 underlined
QAVLTQPSSVSGAPGQRVTISCSGSSSNIGAGYDVHWYQQLPGAAPKLLIYSNNHRPSGVPDR
FSGSKSGTSASLAITGLQTEDEADYYCQSYDRSLSGRVFGGGTKLTVL (SEQ ID NO: 401)
CAGGCTGTGCTGACTCAGCCGTCCTCAGTGTCTGGGGCCCCAGGGCAGAGGGTAACCAT
CTCCTGCAGTGGGAGCAGCTCCAACATCGGGGCAGGTTATGATGTACACTGGTACCAGC
AGCTCCCAGGAGCAGCCCCCAAACTCCTCATCTATAGTAACAATCATCGGCCCTCAGGGG
TCCCTGACCGATTCTCTGGCTCCAAGTCTGGCACCTCAGCCTCCCTGGCCATCACTGGGCT
CCAGACTGAGGATGAGGCTGATTATTACTGCCAGTCCTATGACAGAAGCCTGAGCGGTA
GGGTGTTCGGCGGAGGGACCAAGCTGACCGTCCTA (SEQ ID NO: 402)
Her2_S1R2A_CS_5A1
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAQGTHSSGWSFGYWGQGTLVTVSS (SEQ ID NO: 403)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGCAGGG
TACTCATAGCAGTGGCTGGTCCTTTGGGTACTGGGGCCAGGGCACCCTGGTCACCGTCTC
GAGT (SEQ ID NO: 404)
V_{L} with CDR1, CDR2 and CDR3 underlined
LPVLTQPPSASGTPGQRVTISCSGSSSNIGSKTVNWYQQLPGTTPKLLIYRNNQRPSGVPDRFS
GSKSGTSASLAISGLQSEDEADYYCAAWDDSLNGLIFGGGTKVTVL (SEQ ID NO: 405)
CTGCCTGTGCTGACTCAGCCCCCCTCAGCGTCTGGGACCCCCGGGCAGAGGGTCACCATC
TCTTGTTCTGGAAGCAGCTCCAACATCGGAAGTAAAACTGTAAACTGGTACCAGCAGCTC
CCAGGAACGACCCCCAAACTCCTCATCTATAGGAATAATCAGCGGCCCTCAGGGGTCCCT
GACCGATTCTCTGGCTCCAAGTCTGGCACCTCAGCCTCCCTGGCCATCAGTGGGCTCCAG
TCTGAGGATGAGGCTGATTATTATTGTGCAGCATGGGATGACAGCCTGAATGGTCTGATA
TTCGGCGGAGGGACCAAGGTCACCGTCCTA (SEQ ID NO: 406)
Her2_S1R2A_CS_8C8
V_{H} with CDR1, CDR2 and CDR3 underlined
QVQLVQSGAEVKKPGESLKISCKTSGYSFTSYWIGWVRQMPGKGLEWMGIIYPGDSDTRYSP
SFQGQVTISADKSISTAYLQWSSLKASDTAMYYCARHDLPHQEYQDNGMDVWGKGTLVTV
SS (SEQ ID NO: 407)
CAGGTCCAGCTGGTACAGTCTGGAGCAGAGGTTAAAAAGCCCGGGGAGTCTCTGAAGAT
CTCCTGTAAGACTTCTGGATACAGCTTTACCAGCTATTGGATCGGCTGGGTGCGCCAGAT
GCCCGGGAAAGGCCTGGAGTGGATGGGGATCATCTATCCTGGTGACTCTGATACCAGAT
ACAGCCCGTCTTTTCAAGGCCAGGTCACCATCTCAGCCGACAAGTCCATCAGCACCGCCT
ACCTGCAGTGGAGCAGCCTGAAGGCCTCGGACACCGCCATGTATTACTGTGCGAGACAT
GACCTCCCCCATCAGGAGTATCAGGACAACGGTATGGACGTCTGGGGCAAAGGAACCCT
GGTCACCGTCTCGAGT (SEQ ID NO: 408)
V_{L} with CDR1, CDR2 and CDR3 underlined
QTVVIQEPSFSVSPGETVTLTCALSSGSVSSSYYPSWYQQTPGQAPRALIYNTNTRSSGVPDRF
SGSILGNKAALTITGAQADDESNYYCALYLGSGIWVFGGGTKLTVL (SEQ ID NO: 409)
CAGACTGTGGTGATCCAGGAGCCATCGTTCTCAGTGTCCCCTGGAGAGACAGTCACACTC
ACTTGTGCCTTGAGCTCTGGCTCAGTCTCTAGTAGTTACTACCCCAGCTGGTACCAGCAG
ACCCCAGGCCAGGCTCCACGCGCGCTCATCTACAACACAAACACTCGCTCTTCTGGGGTC
CCTGATCGCTTCTCTGGCTCCATCCTTGGGAACAAAGCTGCCCTCACCATCACGGGGGCC
CAGGCAGATGATGAATCTAACTATTACTGTGCGCTGTATCTGGGTAGTGGCATTTGGGTG
TTCGGCGGAGGGACCAAGCTGACCGTCCTA (SEQ ID NO: 410)
Her2_S1R3A1_CS_13H5
V_{H} with CDR1, CDR2 and CDR3 underlined
EVQLVQSGAEVKKPGESLKISCKGSGYSFPSYWIGWVRQMPGKGLEWMGIIYPGDSETRYSP
SFQGQVTISADKSISTAYLQWSSLKASDTAMYYCVRHLKPVAGPAWHDYGMDVWGQGTLV
TVSS (SEQ ID NO: 411)
GAGGTCCAGCTGGTGCAGTCTGGAGCAGAGGTGAAAAAGCCCGGGGAGTCTCTGAAGAT
CTCCTGTAAGGGTTCTGGATACAGCTTTCCCAGCTACTGGATCGGCTGGGTGCGCCAGAT
GCCCGGGAAAGGCCTGGAGTGGATGGGGATCATCTATCCTGGTGACTCTGAAACCAGAT
ACAGCCCGTCCTTCCAAGGCCAGGTCACCATCTCAGCCGACAAGTCCATCAGCACCGCCT
ACCTGCAGTGGAGCAGCCTGAAGGCCTCGGACACCGCCATGTATTACTGTGTGAGACAT
CTAAAACCAGTGGCTGGTCCCGCTTGGCACGACTACGGTATGGACGTCTGGGGCCAGGG
CACCCTGGTCACCGTCTCGAGT (SEQ ID NO: 412)
V_{L} with CDR1, CDR2 and CDR3 underlined
QAVVLQEPSISVSPGGTVTLTCGLTSDSVSTTYYPSWYQQTPGQTPRTLSYSTNTRSSGVPDR
FSGSILGNKAALTIAGAQADDEADYYCALYMGSGIWVFGGGTQLTVL (SEQ ID NO: 413)
CAGGCTGTGGTGCTCCAGGAGCCATCGATCTCAGTGTCCCCTGGAGGGACAGTCACACTC
ACTTGTGGCTTAACCTCTGACTCAGTCTCGACTACTTACTACCCCAGCTGGTACCAGCAG
ACCCCAGGCCAGACTCCACGCACACTCAGCTACAGCACAAATACTCGCTCTTCTGGGGTC
CCTGATCGCTTCTCTGGCTCCATCCTTGGGAACAAAGCTGCCCTCACCATCGCGGGGGCC
CAGGCAGATGATGAAGCTGATTATTACTGTGCCCTATATATGGGCAGTGGCATTTGGGTG
TTCGGCGGAGGGACCCAGCTCACCGTTTTA (SEQ ID NO: 414)
Her2_S1R2B_CS_5E9
V_{H} with CDR1, CDR2 and CDR3 underlined
QVQLVQSGAEVKKPGESLKISCKGSGYSFANYGIGWVRQMPGKGLEWMGVIYPGDSDIRYS
PSFQGQVIFSADRSISTAYLQWSSLKASDTAMYYCARHLSWLVGGNYGMDVWGKGTMVTV
SS (SEQ ID NO: 415)
CAGGTGCAGCTGGTGCAGTCTGGAGCAGAGGTGAAAAAGCCCGGGGAGTCTCTGAAGAT
CTCCTGTAAGGGTTCTGGATACAGTTTTGCCAACTACGGGATAGGCTGGGTGCGCCAGAT
GCCCGGGAAAGGCCTGGAGTGGATGGGGGTCATCTATCCTGGTGACTCTGATATCAGAT
ACAGCCCGTCCTTCCAAGGCCAGGTCATCTTCTCAGCCGACAGGTCCATCAGCACCGCCT
ACCTGCAGTGGAGCAGCCTGAAGGCCTCGGACACCGCCATGTATTATTGTGCGAGACAT
CTCTCGTGGCTGGTCGGGGGGAACTACGGTATGGACGTCTGGGGCAAAGGGACAATGGT
CACCGTCTCGAGT (SEQ ID NO: 416)
V_{L} with CDR1, CDR2 and CDR3 underlined
QTVVIQEPSFSVSPGGTVTLTCGLTSGSVSTSHYPSWYQQTPGQAPRTLIYSTNTRSSGVPGRF
SGSILGNKAALTITGAQADDESDYYCVLYMGGGISVFGGGTKVTVL (SEQ ID NO: 417)
CAGACTGTGGTGATCCAGGAGCCATCGTTCTCAGTGTCCCCTGGAGGGACAGTCACACTC
ACTTGTGGCTTGACCTCTGGCTCAGTCTCTACTAGTCACTACCCCAGCTGGTACCAGCAG
ACCCCAGGCCAGGCTCCACGCACGCTCATCTACAGCACAAACACTCGCTCTTCTGGGGTC
CCTGGTCGCTTCTCTGGCTCCATCCTTGGGAACAAAGCTGCCCTCACCATCACGGGGGCC
CAGGCAGACGATGAATCTGATTATTATTGTGTGCTGTATATGGGTGGTGGCATTTCGGTG
TTCGGCGGAGGGACCAAGGTCACCGTCCTA (SEQ ID NO: 418)
Her2_S1R3A1_CS_8F9
V_{H} with CDR1, CDR2 and CDR3 underlined
QVQLVQSGAEVKKPGESLKISCKGSGYSFTSQWIAWVRQMPGKGLEWMGIIYPGDSDTRYSP
SFQGQVTISADKSINTAYLQWSSLKASDTAMYYCARHSGSSGDYYHYYGMDVWGQGTMVT
VSS (SEQ ID NO: 419)
CAGGTGCAGCTGGTGCAATCTGGGGCAGAGGTGAAAAAGCCCGGGGAGTCTCTGAAGAT
CTCCTGTAAGGGTTCTGGATACAGCTTTACCAGCCAGTGGATCGCCTGGGTGCGCCAGAT
GCCCGGGAAAGGCCTGGAGTGGATGGGGATCATCTATCCTGGTGACTCTGATACGAGAT
ACAGCCCGTCCTTCCAAGGCCAGGTCACCATCTCAGCCGACAAGTCCATCAACACCGCCT
ACCTGCAGTGGAGCAGCCTGAAGGCCTCGGACACCGCCATGTATTACTGTGCGAGACAT
TCGGGGAGCTCTGGAGATTACTACCACTACTACGGTATGGACGTCTGGGGCCAAGGGAC
AATGGTCACCGTCTCGAGT (SEQ ID NO: 420)
V_{L} with CDR1, CDR2 and CDR3 underlined
QTVVIQEPSFSVSPGGTVTLTCGLSSGSVSTSYYPSWYQQTPGQAPRTLIYSTNTRSSGVPDRF
SGSILGNKAALTITGAQADDESDYYCVLYMGSGISVFGGGTKLTVL (SEQ ID NO: 421)
CAGACTGTGGTGATCCAGGAGCCATCGTTCTCAGTGTCCCCTGGAGGGACAGTCACACTC
ACTTGTGGCTTGAGCTCTGGCTCAGTCTCTACTAGTTACTACCCCAGCTGGTACCAGCAG
ACCCCAGGCCAGGCTCCACGCACGCTCATCTACAGCACAAACACTCGCTCTTCTGGGGTC
CCTGATCGCTTCTCTGGCTCCATCCTTGGGAACAAAGCTGCCCTCACCATCACGGGGGCC
CAGGCAGATGATGAATCTGATTATTACTGTGTGCTGTATATGGGGAGTGGCATTTCGGTG
TTCGGCGGAGGGACCAAGCTGACCGTCCTA (SEQ ID NO: 422)
Her2_S1R3A1_CS_14B5
V_{H} with CDR1, CDR2 and CDR3 underlined
EVQLVQSGAEMKKPGESLKISCKTSGYSFTGSWIAWVRQMPGKGLEWMGIIYPGDSDTRYSP
SFQGQVTISADKSISTAYLQWSSLKASDTAMYYCARIYSDSGYNWFDSWGRGTLVTVSS (SEQ ID NO: 423)
GAGGTGCAGCTGGTGCAGTCTGGGGCAGAGATGAAAAAGCCCGGGGAGTCTCTGAAGAT
ATCCTGCAAGACTTCTGGATACAGCTTTACCGGCTCCTGGATCGCCTGGGTGCGCCAGAT
GCCCGGGAAAGGCCTGGAGTGGATGGGGATCATCTATCCTGGTGACTCTGACACCAGAT
ACAGCCCGTCCTTCCAAGGCCAGGTCACCATCTCAGCCGACAAGTCCATCAGCACCGCCT
ACCTGCAGTGGAGCAGCCTGAAGGCCTCGGACACCGCCATGTATTACTGTGCGAGGATTT
ATAGTGACTCGGGTTACAATTGGTTCGACTCTTGGGGCAGGGGAACCCTGGTCACCGTCT
CGAGT (SEQ ID NO: 424)
V_{L} with CDR1, CDR2 and CDR3 underlined
QTVVIQEPSFSVSPGGTVTLTCGLSSGSVSNSHYPSWYQQTPGQAPRTLIYSTNTRSSGVPDRF
SGSILGNKAALTITGAQADDESDFYCLLYLGSGISVFGGGTKLTVL (SEQ ID NO: 425)
CAGACTGTGGTGATCCAGGAGCCATCGTTCTCAGTGTCCCCTGGAGGGACAGTCACACTC
ACTTGTGGCTTGAGCTCTGGCTCAGTCTCTAATAGTCACTACCCCAGCTGGTATCAGCAG
ACCCCAGGCCAGGCTCCACGAACGCTCATCTACAGCACAAACACTCGCTCTTCTGGGGTC
CCTGATCGCTTCTCTGGCTCCATCCTTGGGAACAAAGCTGCCCTCACCATCACGGGGGCC
CAGGCGGATGATGAATCTGATTTTTACTGTCTGCTATATCTGGGTAGTGGCATTTCGGTAT
TCGGCGGAGGGACCAAGCTGACCGTCCTA (SEQ ID NO: 426)
Her2_S1R2A_CS_9E10
V_{H} with CDR1, CDR2 and CDR3 underlined
EVQLVQSGAEVKKPGESLKISCQGSGYTFASQWIAWVRQMPGQGLEWMGTIWPGDSNPTYS
PSFQGQVTISADKSISTAYLQWSSLKASDTAIYYCARLYNNYPYFYGMDVWGQGTMVTVSS (SEQ ID NO: 427)
GAGGTGCAGCTGGTGCAGTCTGGAGCAGAGGTGAAGAAGCCCGGGGAGTCTCTGAAGAT
CTCCTGTCAGGGTTCTGGATACACCTTTGCCAGCCAATGGATCGCCTGGGTGCGCCAGAT
GCCCGGGCAAGGCCTGGAGTGGATGGGGACCATCTGGCCTGGTGACTCTAATCCCACAT
ATAGCCCGTCCTTCCAAGGCCAGGTCACCATCTCAGCCGACAAGTCCATCAGCACCGCCT
ACCTGCAGTGGAGCAGCCTGAAGGCCTCGGACACCGCCATCTATTACTGTGCGAGGCTCT
ACAATAACTATCCCTACTTCTACGGTATGGACGTCTGGGGCCAGGGGACAATGGTCACCG
TCTCGAGT (SEQ ID NO: 428)
V_{L} with CDR1, CDR2 and CDR3 underlined
QTVVLQEPSFSVSPGGTVTLTCGLRSGSVSTTYYPSWYQQTPGQAPRTLIYSTNTRSSGVPDR
FSGSIVGNKAALTITGAQADDESDYYCALYLGSGTWVFGGGTKLTVL (SEQ ID NO: 429)
CAGACTGTGGTGCTCCAGGAGCCATCGTTCTCAGTGTCCCCTGGAGGGACAGTCACACTC
ACTTGTGGCTTGAGGTCTGGCTCAGTCTCTACTACTTACTACCCCAGCTGGTACCAGCAG
ACCCCAGGCCAGGCTCCACGCACGCTCATCTACAGCACAAACACTCGCTCTTCTGGGGTC
CCTGATCGCTTCTCTGGCTCCATCGTCGGGAACAAAGCTGCCCTCACCATCACGGGGGCC
CAGGCAGATGATGAATCTGATTATTACTGTGCGCTATACCTGGGTAGTGGCACTTGGGTG
TTCGGCGGAGGGACCAAGCTGACCGTCCTA (SEQ ID NO: 430)
Her2_S1R3A1_CS_7A10
V_{H} with CDR1, CDR2 and CDR3 underlined
EVQLVQSGAEVKKSGESLKISCKGSGYSFTSNWIGWVRQMPGKGLEWMGIIYPGDSDTRYSP
SFQGQVTISADKSVSTAYLQWSSLKASDTAMYYCARMLTDCSSTSCYSAGMDVWGKGTLV
TVSS (SEQ ID NO: 431)
GAGGTCCAGCTGGTGCAGTCTGGGGCAGAGGTGAAAAAGTCCGGGGAGTCTCTGAAGAT
CTCCTGTAAGGGTTCTGGATACAGCTTTACCAGTAATTGGATCGGCTGGGTGCGCCAGAT
GCCCGGGAAAGGCCTGGAGTGGATGGGGATCATCTATCCTGGTGACTCTGATACCAGAT
ACAGCCCGTCCTTCCAAGGCCAGGTCACCATCTCAGCCGACAAGTCCGTCAGCACCGCCT
ACCTGCAGTGGAGCAGCCTGAAGGCCTCGGACACCGCCATGTATTATTGTGCGAGAATG
CTGACGGACTGTAGTAGTACCAGCTGCTATTCAGCCGGTATGGACGTCTGGGGCAAAGG
CACCCTGGTCACCGTCTCGAGT (SEQ ID NO: 432)
V_{L} with CDR1, CDR2 and CDR3 underlined
QAVVIQEPSFSVSPGGTVTLTCGLSSGSVSPSYYPSWYQQTPGQAPRTLIYSTNTRSSGVPDRF
SGSILGNKAALTITGAQADDESDYYCVLYMGSGSWVFGGGTKLTVL (SEQ ID NO: 433)
CAGGCTGTGGTGATCCAGGAGCCATCGTTCTCAGTGTCCCCTGGAGGGACAGTCACACTC
ACTTGTGGCTTGAGTTCTGGCTCAGTCTCTCCTAGTTACTACCCCAGCTGGTACCAGCAG
ACCCCAGGCCAGGCTCCACGCACACTCATCTACAGCACAAACACTCGCTCTTCTGGGGTC
CCTGATCGCTTCTCTGGCTCCATCCTTGGGAACAAAGCTGCCCTCACCATCACGGGGGCC
CAGGCAGATGATGAATCTGATTATTACTGTGTGCTGTATATGGGTAGTGGCTCTTGGGTG
TTCGGCGGAGGGACCAAGCTGACCGTCCTA (SEQ ID NO: 434)
Her2_S1R3A1_BMV_6H7
V_{H} with CDR1, CDR2 and CDR3 underlined
QVQLVESGGGVVQPGRSLRLSCAASGFTFSSYAMNWVRQAPGKGLEWVATISYDGSNKYY
ADSVKGRFTISRDNSKNTLYLQMNSLRAEDTAVYYCARPAPYSSSGAFDIWGQGTLVTVSS (SEQ ID NO: 435)
CAGGTGCAGCTGGTGGAGTCTGGGGGAGGCGTGGTCCAGCCTGGGAGGTCCCTGAGACT
CTCCTGTGCAGCCTCTGGATTCACCTTCAGTAGCTATGCTATGAACTGGGTCCGCCAGGC
TCCAGGCAAGGGGCTGGAGTGGGTGGCAACTATATCATATGATGGAAGCAATAAATACT
ACGCAGACTCCGTGAAGGGCCGATTCACCATCTCCAGAGACAATTCCAAGAACACGCTG
TATCTGCAAATGAACAGCCTGAGAGCCGAGGACACGGCTGTGTATTACTGTGCGAGACC
GGCCCCGTATAGCAGCTCCGGCGCTTTTGATATCTGGGGCCAAGGCACCCTGGTCACCGT
CTCTTCA (SEQ ID NO: 436)
V_{L} with CDR1, CDR2 and CDR3 underlined
DIQMTQSPSTLSASIGDRVTITCRASEGIYHWLAWYQQKPGKAPKLLTYKASSLASGAPSRFS
GSGSGTDFTLTISSLQPDDFATYYCQQYSNYPLT FGGGTKLEIKR (SEQ ID NO: 437)
GACATCCAGATGACCCAGTCTCCTTCCACCCTGTCTGCATCTATTGGAGACAGAGTCACC
ATCACCTGCCGGGCCAGTGAGGGTATTTATCACTGGTTGGCCTGGTATCAGCAGAAGCCA
GGGAAAGCCCCTAAACTCCTGACCTATAAGGCCTCTAGTTTAGCCAGTGGGGCCCCATCA
AGGTTCAGCGGCAGTGGATCTGGGACAGATTTCACTCTCACCATCAGCAGCCTGCAGCCT
GATGATTTTGCAACTTATTACTGCCAACAATATAGTAATTATCCGCTCACTTTCGGCGGA
GGGACCAAGCTGGAGATCAAACGT (SEQ ID NO: 438)
Her2_S1R3A1_CS_12A11
V_{H} with CDR1, CDR2 and CDR3 underlined
QVQLVQSGGEVKKPGESLKISCKVSGDKFANYWIAWVRQVPGRGLEWMGIIYPSDSDVRYS
PSFQGQVTMSADKSTSTAYLQLSSLKASDTAMYYCARQVGGLVTTDTDSYFYGMDVWGQG
TLVTVSS (SEQ ID NO: 439)
CAGGTCCAGCTGGTGCAGTCTGGAGGAGAGGTGAAAAAGCCCGGGGAGTCTCTGAAGAT
CTCCTGTAAGGTTTCTGGAGACAAGTTTGCCAACTACTGGATCGCCTGGGTGCGCCAGGT
GCCCGGGAGAGGCCTGGAGTGGATGGGAATCATCTATCCTAGTGACTCTGATGTCAGAT
ATAGTCCGTCCTTCCAAGGCCAAGTCACCATGTCAGCCGACAAGTCCACCAGCACCGCCT
ACTTGCAGTTGAGCAGCCTGAAGGCCTCGGACACCGCCATGTATTACTGTGCAAGACAG
GTGGGTGGACTGGTTACTACAGACACTGACTCCTACTTCTACGGCATGGACGTCTGGGGC
CAAGGAACCCTGGTCACCGTCTCGAGT (SEQ ID NO: 440)
V_{L} with CDR1, CDR2 and CDR3 underlined
QAVVIQEPSFSVSPGGTVTLTCGLSSGPVSTSYYPSWFQQTPGQAPRTLIYSTNTRSSGVPDRF
SGSILGNKAALTITGAQADDESDYYCVLYVGSGISLFGGGTKVTVL (SEQ ID NO: 441)
CAGGCTGTGGTGATCCAGGAGCCATCGTTCTCAGTGTCCCCTGGAGGGACAGTCACACTC
ACTTGTGGCTTGAGCTCTGGCCCAGTCTCTACTAGTTACTACCCCAGCTGGTTCCAACAG
ACCCCAGGCCAGGCTCCACGCACGCTCATCTACAGCACAAACACTCGCTCTTCTGGGGTC
CCTGATCGCTTCTCTGGCTCCATCCTTGGGAACAAAGCTGCCCTCACCATCACGGGGGCC
CAGGCAGATGATGAATCTGATTATTACTGTGTGTTGTATGTGGGTAGTGGCATTTCGCTA
TTCGGCGGGGGGACCAAGGTCACCGTCCTA (SEQ ID NO: 442)
Her2_S1R3A1_CS_13D12
V_{H} with CDR1, CDR2 and CDR3 underlined
QVQLVQSGGEVKKPGESLKISCKVSGDSFTNYWIAWVRQMPGRGLEWMGIIYPSDSDVRYS
PSFQGQVTMSADKSISTAYLQLSSLKASDTAMYYCARQVGGLVTTDTDSYFYGMDVWGRG
TLVTVSS (SEQ ID NO: 443)
CAGGTCCAGCTGGTGCAGTCTGGAGGAGAGGTGAAAAAGCCCGGGGAGTCTCTGAAGAT
CTCCTGTAAGGTTTCTGGAGACAGCTTTACCAACTACTGGATCGCCTGGGTGCGCCAGAT
GCCCGGGAGAGGCCTGGAGTGGATGGGAATCATCTATCCTAGTGACTCTGATGTCAGAT
ATAGTCCGTCCTTCCAAGGCCAGGTCACCATGTCAGCCGACAAGTCCATCAGCACCGCCT
ACCTGCAGTTGAGCAGCCTGAAGGCCTCGGACACCGCCATGTATTACTGTGCAAGACAG
GTGGGTGGACTGGTTACTACAGACACTGACTCCTACTTCTACGGCATGGACGTCTGGGGC
AGAGGCACCCTGGTCACCGTCTCGAGT (SEQ ID NO: 444)
V_{L} with CDR1, CDR2 and CDR3 underlined
QTVVIQEPSFSVSPGGTVTLTCALNSGSVSTNYYPSWYQQTPGQAPRTLIHSTNTRSSGVPDRF
SGSILGNNAALTITGAQAEDESDYYCALYMGSGISIFGGGTKLTVL (SEQ ID NO: 445)
CAGACTGTGGTGATCCAGGAGCCATCGTTCTCAGTGTCCCCTGGAGGGACAGTCACACTC
ACTTGTGCCTTGAACTCCGGCTCAGTCTCTACTAATTACTACCCCAGCTGGTACCAGCAG
ACCCCAGGCCAGGCTCCACGCACGCTCATCCACAGCACAAACACTCGCTCTTCTGGGGTC
CCTGATCGCTTCTCTGGCTCCATCCTTGGGAACAATGCTGCCCTCACCATCACGGGGGCC
CAGGCAGAGGATGAATCTGATTATTACTGTGCGCTATATATGGGTAGTGGCATTTCGATA
TTCGGCGGAGGGACCAAGCTGACCGTCCTA (SEQ ID NO: 446)
Her2_S1R3A1_CS_7A8
V_{H} with CDR1, CDR2 and CDR3 underlined
QVQLQESGPGLVEPSETLSLTCSVSGGSISSSSSSWGWIRQPPGKGLEWIGSIYYSGETYYNPS
LKRRVTISTDTSKNQLSLELASVTAADTAVYYCARQVTSYGSDYFDYWGKGTLVTVSS (SEQ ID NO: 447)
CAGGTGCAGCTGCAGGAGTCGGGCCCAGGACTGGTGGAGCCTTCGGAGACCCTGTCCCT
CACCTGCAGTGTCTCTGGCGGCTCCATCAGCAGCAGTAGTTCCTCTTGGGGCTGGATCCG
CCAGCCCCCAGGGAAGGGGCTGGAGTGGATTGGGAGTATCTATTACAGTGGAGAAACCT
ATTATAATCCGTCCCTCAAGAGGCGTGTCACCATATCCACAGACACGTCCAAGAACCAGT
TGTCCCTGGAGCTGGCCTCTGTGACCGCCGCAGACACGGCTGTATATTACTGTGCGAGGC
AAGTCACCAGTTATGGTTCTGACTACTTTGACTACTGGGGCAAAGGAACCCTGGTCACCG
TCTCGAGT (SEQ ID NO: 448)
V_{L} with CDR1, CDR2 and CDR3 underlined
QAVVIQEPSFSVSPGGTVTLTCGLSSGSVSSNYYPSWYQQTPGQTPRTLIYNTNTRSSGVPDRF
SGSILGNKAALTITGAQADDESDYYCVLYMGSGIRVFGGGTKVTVL (SEQ ID NO: 449)
CAGGCTGTGGTGATCCAGGAGCCATCGTTCTCAGTGTCCCCTGGAGGGACAGTCACACTT
ACTTGTGGCTTGAGCTCTGGCTCAGTCTCTTCTAATTACTACCCCAGCTGGTACCAGCAG
ACCCCAGGCCAGACTCCACGCACGCTCATCTACAACACAAACACTCGCTCTTCTGGGGTC
CCTGATCGCTTCTCTGGCTCCATCCTTGGGAACAAAGCTGCCCTCACCATCACGGGGGCC
CAGGCAGATGATGAATCTGATTATTACTGTGTGTTGTATATGGGTAGTGGCATTCGCGTG
TTCGGCGGAGGGACCAAGGTCACCGTCCTA (SEQ ID NO: 450)
Her2_S1R2A_CS_2C9
V_{H} with CDR1, CDR2 and CDR3 underlined
EVQLVQSGAEVRKPGASVKVSCRSSGYTFTTYYLHWLRQAPGQGLEWMGVINPSGGATAY
AQSFQGRVTMTRDTATSTVYLDLSSLRTEDTAVYYCARSTPAEQLVPGFWGKGTMVTVSS (SEQ ID NO: 451)
GAAGTGCAGCTGGTGCAGTCTGGGGCTGAGGTGAGGAAGCCTGGGGCCTCAGTGAAGGT
TTCCTGCAGGTCATCTGGATACACCTTCACCACCTACTATTTGCACTGGCTACGACAGGC
CCCTGGACAAGGGCTTGAGTGGATGGGAGTAATTAACCCTAGCGGCGGTGCCACAGCCT
ACGCGCAGAGTTTCCAGGGCAGAGTCACCATGACCAGGGACACGGCTACGAGCACAGTC
TATTTGGATCTGAGCAGCCTGAGAACTGAAGACACGGCCGTGTATTACTGTGCGAGATCC
ACCCCGGCGGAGCAGCTCGTCCCGGGCTTCTGGGGCAAAGGGACAATGGTCACCGTCTC
GAGT (SEQ ID NO: 452)
V_{L} with CDR1, CDR2 and CDR3 underlined
EIVMTQSPATLSVSPGDRATLSCRASQSVSTNVAWYQQKPGQPPRLLIYGASTRASGVPARFS
GSGSGTEFTLTISSLQSEDFAVYYCQQYGDWPPITFGQGTRLEIKR (SEQ ID NO: 453)
GAAATTGTGATGACGCAGTCTCCAGCCACCCTGTCTGTGTCTCCAGGGGACAGAGCCACC
CTCTCCTGCAGGGCCAGTCAGAGTGTTAGCACCAACGTAGCCTGGTACCAGCAGAAACC
TGGCCAACCTCCCAGGCTCCTCATCTATGGTGCATCCACCAGGGCCTCTGGTGTCCCAGC
CAGGTTCAGTGGCAGTGGGTCTGGGACAGAGTTCACTCTCACCATCAGCAGTCTGCAGTC
TGAAGATTTTGCAGTTTATTACTGTCAACAGTATGGTGACTGGCCTCCGATCACCTTCGG
CCAAGGGACACGACTGGAGATTAAACGT (SEQ ID NO: 454)
Her2_S1R3A1_CS_12D1
V_{H} with CDR1, CDR2 and CDR3 underlined
EVQLVESGGGLVQPGGSLKLSCAASGLNFDISTVHWVRQASGKGLEWIGRIRSKAYNYATA
YTESLKGRFIISRDESKNTADLQINSLKTEDTATYYCSMTFGDYYYYGMDVWGRGTLVTVSS (SEQ ID NO: 455)
GAGGTGCAGCTGGTGGAGTCCGGGGGAGGCTTGGTCCAGCCGGGGGGGTCCCTAAAACT
TTCCTGTGCAGCCTCTGGGCTCAATTTCGATATCTCTACTGTGCACTGGGTCCGCCAGGCT
TCCGGGAAAGGGCTGGAGTGGATTGGCCGTATTAGAAGCAAAGCTTACAATTATGCGAC
AGCATATACTGAGTCGCTGAAGGGCAGGTTCATCATCTCCAGAGATGAGTCAAAGAATA
CGGCGGATCTGCAAATCAACAGCCTGAAAACCGAGGACACGGCCACATATTACTGTAGT
ATGACCTTCGGTGACTACTACTACTACGGCATGGACGTCTGGGGCCGGGGCACCCTGGTC
ACCGTCTCGAGT (SEQ ID NO: 456)
V_{L} with CDR1, CDR2 and CDR3 underlined
QAVLTQPSSVSGAPGQRVTITCTGSSSNIGAGYDVHWYQQLPGTAPKLLIYSNSYRPSGVSDR
FSGSKSGTSASLVIAGLQAEDEADYYCQSYDSSHWFFGGGTKLTVL (SEQ ID NO: 457)
CAGGCTGTGCTGACTCAGCCGTCCTCAGTGTCTGGGGCCCCAGGGCAGAGGGTCACCATC
ACCTGCACTGGAAGCAGCTCCAACATCGGGGCCGGTTACGATGTTCACTGGTACCAGCA
ACTTCCAGGAACAGCCCCCAAACTCCTCATCTATAGTAATTCTTATCGGCCCTCTGGGGT
CTCTGACCGATTCTCTGGCTCCAAGTCTGGCACCTCAGCCTCCCTGGTCATCGCTGGACTC
CAGGCTGAGGATGAGGCTGATTATTACTGTCAGTCCTATGACAGCAGTCATTGGTTTTTC
GGCGGAGGGACCAAGCTGACCGTCCTA (SEQ ID NO: 458)
Her2_S1R2A_CS_7D4
V_{H} with CDR1, CDR2 and CDR3 underlined
QVQLQQSGAEVKKPGASVKVSCKVSGYTPPDLSIHWVRQAPGEGLEWMGRFDFEDGETINA
QKFQGRVTMTADTSTNTGYMEVSSLRFEDTAVYYCATTLRFSGYYYGMDFWGRGTLVTVSS (SEQ ID NO: 459)
CAGGTACAGCTGCAGCAGTCAGGGGCTGAGGTGAAGAAGCCTGGGGCCTCAGTGAAGGT
CTCCTGCAAGGTTTCCGGATACACCCCCCCTGATTTATCCATCCACTGGGTGCGACAGGC
TCCTGGAGAAGGGCTTGAGTGGATGGGACGTTTTGATTTTGAAGATGGTGAAACAATCA
ACGCACAGAAGTTCCAGGGCAGAGTCACCATGACCGCGGACACATCCACAAACACAGGC
TACATGGAGGTGAGCAGCCTGAGATTTGAGGACACGGCCGTGTATTACTGTGCAACAAC
ACTTCGATTTTCTGGTTACTACTACGGTATGGACTTCTGGGGCCGAGGAACCCTGGTCAC
CGTCTCGAGT (SEQ ID NO: 460)
V_{L} with CDR1, CDR2 and CDR3 underlined
QAVVIQEPSLSVSPGGTVTLTCALSSGSVSTGYYPSWYQQTPGQAPRTLIYNTDTRSSGVPGR
FSGSILGDKAALTITGAQADDESDYYCVLYMGSGIWVFGGGTKVTVL (SEQ ID NO: 461)
CAGGCTGTGGTGATCCAGGAGCCATCGCTCTCAGTGTCCCCTGGAGGGACAGTCACACTC
ACTTGTGCCTTGAGCTCTGGCTCAGTCTCCACTGGTTACTACCCCAGCTGGTACCAGCAG
ACCCCAGGCCAGGCTCCACGCACGCTCATTTACAACACAGACACTCGCTCTTCTGGGGTC
CCTGGTCGCTTCTCTGGCTCCATCCTTGGGGACAAAGCTGCCCTCACCATCACGGGGGCC
CAGGCAGATGATGAATCTGATTATTACTGTGTGCTGTATATGGGTAGTGGCATTTGGGTG
TTCGGCGGAGGGACCAAGGTCACCGTCCTA (SEQ ID NO: 462)
Her2_S1R3A1_CS_15B8
V_{H} with CDR1, CDR2 and CDR3 underlined
EVQLVQSGAEVKKPGESLKISCKGSGYNFNTHWIGWVRQMPGKGLEWMGLIYPDDSDTRFS
PSFEGQVTLSADRSISTAYLQWTSLKASDTAMYYCARYKKSSGYYTGYGMDVWGRGTMVT
VSS (SEQ ID NO: 463)
GAAGTGCAGCTGGTGCAGTCTGGAGCAGAGGTGAAAAAGCCCGGGGAGTCTCTGAAGAT
CTCCTGTAAGGGTTCTGGATACAACTTTAACACTCACTGGATCGGGTGGGTGCGCCAGAT
GCCCGGGAAAGGCCTGGAGTGGATGGGGCTCATCTACCCTGATGACTCTGACACCCGAT
TCAGCCCGTCCTTCGAAGGCCAGGTCACCCTCTCAGCCGACAGGTCCATCAGTACCGCCT
ACCTGCAGTGGACCAGCCTGAAGGCCTCGGACACCGCCATGTATTACTGTGCGAGATAC
AAAAAAAGTAGTGGTTATTACACAGGATATGGTATGGACGTCTGGGGCCGAGGGACAAT
GGTCACCGTCTCGAGT (SEQ ID NO: 464)
V_{L} with CDR1, CDR2 and CDR3 underlined
QTVVIQEPSFSVSPGGTVTLTCGLSSGSVSTSYYPTWYQQTPGQAPRTLIYSTNSRFSGVPDRF
SGSILGSKAALTITGAQADDESDYYCVLYMGSGISVFGGGTKVTVL (SEQ ID NO: 465)
CAGACTGTGGTGATCCAGGAGCCATCGTTCTCAGTGTCCCCTGGAGGGACAGTCACACTC
ACTTGTGGCTTGAGCTCTGGCTCAGTCTCTACTAGTTACTACCCCACCTGGTACCAGCAG
ACCCCAGGCCAGGCTCCACGCACGCTCATCTATAGCACAAACAGTCGCTTTTCTGGGGTC
CCTGATCGCTTCTCTGGCTCCATCCTTGGGAGCAAAGCTGCCCTCACCATCACGGGGGCC
CAGGCAGATGATGAATCTGATTATTACTGTGTGCTATATATGGGTAGTGGCATTTCGGTG
TTCGGCGGAGGGACCAAGGTCACCGTCCTA (SEQ ID NO: 466)
Her2_S6R3_DP47_1A10
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCATSLGYGDFDYWGRGTTVTVSS (SEQ ID NO: 467)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCAACTTC
GTTGGGTTACGGTGACTTTGACTACTGGGGGCGAGGGACCACGGTTACCGTCTCGAGT (SEQ ID NO: 468)
V_{L} with CDR1, CDR2 and CDR3 underlined
QAVLTQPSSASGAPGHRVIISCSGSSSNIGSYYVSWYQQLPGAAPKLLIYRNDERPSGVPARFS
GSTSGTSASLAISGLHSEDEADYYCAAWDDSLNGPVFGGGTKVTVL (SEQ ID NO: 469)
CAGGCTGTGCTGACTCAGCCGTCCTCAGCGTCTGGGGCCCCCGGGCACAGGGTCATCATC
TCTTGTTCTGGAAGCAGCTCCAACATCGGAAGTTATTATGTAAGCTGGTACCAGCAGCTC
CCAGGAGCGGCCCCCAAACTCCTCATCTATCGTAATGATGAGCGGCCCTCAGGGGTCCCT
GCCCGATTCTCTGGCTCCACGTCTGGCACCTCAGCCTCCCTGGCCATCAGTGGGCTCCAC
TCTGAGGATGAGGCTGATTATTATTGTGCAGCATGGGATGACAGCCTGAATGGTCCGGTT
TTCGGCGGAGGGACCAAGGTCACCGTCCTA (SEQ ID NO: 470)
Her2_S6R2_DP47_1E11
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAVHGYGDSVDDALDVWGRGTLVTVSS (SEQ ID NO: 471)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGGTCCA
TGGCTACGGAGACTCCGTGGATGATGCTCTTGATGTCTGGGGCCGAGGAACCCTGGTCAC
CGTCTCGAGT (SEQ ID NO: 472)
V_{L} with CDR1, CDR2 and CDR3 underlined
QSVLTQPPSASGTPGQTISISCSGSNSNIGTYSVSWYQQLPRAAPRLLVYANDRRPSGVPDRFS
GSKSGTSASLAISGLQSEDEADYYCAVWDDRLNGFVFGTGTKLTVL (SEQ ID NO: 473)
CAGTCTGTGCTGACGCAGCCGCCCTCAGCGTCTGGGACCCCCGGGCAGACGATCTCCATC
TCTTGTTCTGGAAGCAACTCCAACATCGGAACTTATAGTGTTAGCTGGTACCAGCAGCTC
CCACGAGCGGCCCCCAGACTCCTCGTCTATGCTAATGATCGCCGGCCCTCAGGGGTCCCT
GACCGATTCTCTGGCTCCAAGTCTGGCACCTCAGCCTCCCTGGCCATCAGTGGGCTCCAG
TCTGAGGATGAGGCTGATTATTACTGTGCAGTATGGGATGACAGGTTGAATGGTTTTGTC
TTCGGAACTGGGACCAAGCTGACCGTCCTA (SEQ ID NO: 474)
Her2_S5R2_DP47_1H11
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARDDDFWSGYPFLYYYYGMDVWGRG
TMVTVSS (SEQ ID NO: 475)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGA
TGACGATTTTTGGAGTGGTTATCCATTCCTCTACTACTACTACGGTATGGACGTCTGGGGC
CGAGGGACAATGGTCACCGTCTCGAGT (SEQ ID NO: 476)
V_{L} with CDR1, CDR2 and CDR3 underlined
QSVVTQPPSASGTPGQRVTISCSGTSSNIGSNAVNWYQQLPGTAPKLLIYNNNQRPSGVPDRF
SGSKSGTSASLAISGLQSEDEADYYCAAWDDSLNVYVVFGGGTKLTVL (SEQ ID NO: 477)
CAGTCTGTCGTGACGCAGCCGCCCTCAGCGTCTGGGACCCCCGGGCAGAGGGTCACCAT
CTCTTGTTCTGGAACTAGTTCCAACATCGGAAGTAATGCTGTAAACTGGTACCAGCAACT
CCCAGGAACGGCCCCCAAACTCCTCATCTATAATAATAATCAGCGGCCCTCAGGGGTCCC
TGACCGATTCTCTGGCTCCAAGTCTGGCACCTCAGCCTCCCTGGCCATCAGTGGACTCCA
GTCTGAGGATGAGGCTGATTATTACTGTGCAGCATGGGATGACAGCCTGAATGTTTATGT
GGTATTCGGCGGAGGGACCAAGCTGACCGTCCTA (SEQ ID NO: 478)
Her2_S6R3_CS_1G5
V_{H} with CDR1, CDR2 and CDR3 underlined
QVQLQESGPGLVKPSETLSLTCTVSGGSITSDLSYWGWLRQPPGKGLEWIASGGDGESTYYN
PSLNGRVTFSVDTPKNQFSLRLSSVTAADTAVYYCARHPLYYCSGGRCYSGNFDFWGQGTL
VTVSS (SEQ ID NO: 479)
CAGGTGCAGCTGCAGGAGTCGGGCCCAGGACTGGTGAAGCCTTCGGAGACCCTGTCCCT
CACCTGCACTGTCTCTGGTGGCTCCATCACCAGTGATCTTTCCTACTGGGGCTGGCTCCGC
CAGCCCCCCGGGAAGGGTCTGGAGTGGATTGCGAGTGGTGGTGACGGTGAGAGCACCTA
CTACAACCCGTCCCTCAACGGTCGAGTCACCTTTTCCGTGGACACGCCCAAGAACCAATT
CTCCCTGAGGCTGAGCTCTGTGACCGCCGCAGACACGGCTGTATATTACTGTGCGAGACA
CCCACTCTACTATTGTAGTGGTGGTCGCTGCTACTCCGGGAACTTTGACTTTTGGGGCCA
GGGAACCCTGGTCACCGTCTCGAGT (SEQ ID NO: 480)
V_{L} with CDR1, CDR2 and CDR3 underlined
QAVLTQPSSASGTPGQRVTISCSGTTPNIGSNFVYWYQQLPGTAPKLLIYRNEQRPSGVPVRFS
GSKSGTSASLAISDLRSEDEADYYCAAWDDSLSGVVFGGGTKLTVL (SEQ ID NO: 481)
CAGGCTGTGCTGACTCAGCCGTCCTCAGCGTCTGGGACCCCCGGTCAGAGGGTCACCATT
TCTTGTTCTGGAACGACCCCCAATATTGGAAGTAATTTTGTCTACTGGTATCAACAACTCC
CAGGGACGGCCCCCAAACTCCTCATCTACAGGAATGAGCAGCGCCCTTCAGGGGTCCCT
GTCCGATTCTCTGGCTCCAAGTCTGGCACATCAGCCTCCCTGGCCATCAGTGACCTCCGG
TCCGAGGATGAGGCTGACTATTACTGTGCAGCGTGGGATGACAGCCTGAGTGGTGTGGT
CTTCGGCGGGGGGACCAAGCTGACCGTCCTA (SEQ ID NO: 482)
Her2_S6R2_DP47_1H11
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKYGGYDADAFDVWGRGTMVTVSS (SEQ ID NO: 483)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAAGTA
TGGTGGCTACGACGCTGATGCCTTTGATGTCTGGGGCCGAGGGACAATGGTCACCGTCTC
GAGT (SEQ ID NO: 484)
V_{L} with CDR1, CDR2 and CDR3 underlined
QSVVTQPPSVSAAPGQKVTISCSGSSSNIGDYYVSWYQQLPGTAPTLLIYDNDKRPSEVPDRF
SGSKSGTSATLGITGLQTGDEADYYCTSWDSSLSAGVFGGGTKVTVL (SEQ ID NO: 485)
CAGTCTGTCGTGACGCAGCCGCCCTCAGTGTCTGCGGCCCCAGGACAGAAGGTCACCATC
TCCTGCTCTGGAAGTAGCTCCAACATTGGAGATTATTATGTATCCTGGTACCAGCAACTC
CCAGGAACGGCCCCCACACTCCTCATTTATGACAATGATAAGCGACCCTCAGAAGTTCCT
GACCGATTCTCTGGCTCCAAGTCTGGCACGTCGGCCACCCTCGGCATCACCGGACTCCAG
ACTGGGGACGAGGCCGATTATTACTGCACTTCATGGGATAGCAGCCTGAGTGCTGGGGT
GTTCGGCGGAGGGACCAAGGTCACCGTCCTA (SEQ ID NO: 486)
Her2_S5R3_DP47_1A10
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKWGWDYYDTTGHDAFDFWGRGTM
VTVSS (SEQ ID NO: 487)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACTTACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCTAAATG
GGGCTGGGATTACTATGACACAACTGGTCATGATGCCTTTGATTTCTGGGGCCGGGGGAC
AATGGTCACCGTCTCGAGT (SEQ ID NO: 488)
V_{L} with CDR1, CDR2 and CDR3 underlined
QAVLTQPSSVSGAPGQRVTISCTGSSSNIGADYYVNWYQQLPGKAPEIVIFNDDNRPSGVPNR
FSGSKSGTSASLAITGLQAEDEADYYCQSYDSVLSAYVFGTGTKVTVL (SEQ ID NO: 489)
CAGGCTGTGCTGACTCAGCCGTCCTCAGTGTCTGGGGCCCCAGGGCAGAGGGTCACCATC
TCCTGCACTGGGAGCAGCTCCAACATCGGGGCAGATTATTATGTAAATTGGTATCAGCAA
CTTCCAGGAAAAGCCCCCGAAATCGTAATTTTTAATGATGACAATCGGCCCTCAGGGGTC
CCTAACCGATTCTCTGGCTCCAAGTCTGGCACCTCAGCCTCCCTGGCCATCACTGGGCTC
CAGGCTGAAGATGAGGCTGATTATTATTGCCAGTCTTATGACAGTGTCCTGAGTGCTTAT
GTCTTCGGAACTGGGACCAAGGTCACCGTCCTA (SEQ ID NO: 490)
Her2_S5R2_DP47_1D11
V_{H} with CDR1, CDR2 and CDR3 underlined
EVQLLESGGGLVQPGGSLRLSCAASGFTFSSYTMSWVRQAPGKGLEWVSAISGSGGSTYYAD
SVKGRFTISRDNSKNTLYLQMNSLRAEDTAVYYCARNSSNWSGAFDIWGRGTTVTVSS (SEQ ID NO: 491)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATACCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCAAGGAA
TAGCAGCAATTGGAGTGGTGCTTTTGATATCTGGGGGCGGGGGACCACGGTCACCGTCTC
GAGT (SEQ ID NO: 492)
V_{L} with CDR1, CDR2 and CDR3 underlined
QSVLTQPPSVSGAPGQRVTISCIGTHSNIGAGYAVNWYQQLPGTAPKLLIYGNNNRPSGVPDR
FSGSKSGTSASLAINGLQADDESDYYCQSYDASLRVLFGGGTKLTVL (SEQ ID NO: 493)
CAGTCTGTGCTGACGCAGCCGCCCTCAGTGTCTGGGGCCCCAGGGCAGAGGGTCACCAT
CTCCTGCATTGGAACCCACTCAAACATCGGGGCAGGTTACGCTGTGAACTGGTACCAGCA
GCTTCCAGGAACAGCCCCCAAACTCCTCATCTATGGTAATAACAATCGGCCCTCAGGGGT
CCCTGACCGATTCTCTGGCTCCAAGTCTGGCACCTCAGCCTCCCTGGCCATCAATGGGCT
CCAGGCTGACGATGAGTCTGATTATTATTGCCAGTCCTATGACGCCAGTCTGAGAGTTTT
ATTCGGCGGAGGGACCAAGCTGACCGTCCTA (SEQ ID NO: 494)
Her2_S5R2_CS_1A8
V_{H} with CDR1, CDR2 and CDR3 underlined
EVQLVQSGAEVKKPGESLKISCKGSGYSFSNYWIGWVRQMPGKGLEWMGIIYPGDSDTRYSP
SFQGQVTISADKSISTAYLQWSSLKASDSAMYYCARHDSTMGYDAFHMWGQGTLVTVSS (SEQ ID NO: 495)
GAGGTGCAGCTGGTGCAGTCTGGGGCAGAGGTGAAAAAGCCCGGGGAGTCTCTGAAGAT
CTCCTGTAAGGGTTCTGGGTACAGCTTTAGTAACTACTGGATCGGCTGGGTGCGCCAGAT
GCCCGGGAAAGGCCTGGAGTGGATGGGGATCATCTATCCTGGTGACTCTGATACCAGAT
ACAGCCCGTCCTTCCAAGGCCAGGTCACCATCTCAGCCGACAAGTCCATCAGTACCGCCT
ACCTGCAGTGGAGCAGCCTGAAGGCCTCGGACAGTGCCATGTATTACTGTGCGAGACAT
GATTCGACTATGGGATATGATGCTTTTCATATGTGGGGCCAAGGAACCCTGGTCACCGTC
TCGAGT (SEQ ID NO: 496)
V_{L} with CDR1, CDR2 and CDR3 underlined
QAVLTQPSSVSGAPGQRVTISCTGSSSNIGAGYDVHWYQQLPGTAPKLLIYGNSNRPSGVPDR
FSGSKSGTSASLAITGLQAEDEADYYCQSYDSSLSGPVVFGGGTKLTVL (SEQ ID NO: 497)
CAGGCTGTGCTGACTCAGCCGTCCTCAGTGTCTGGGGCCCCAGGGCAGAGGGTCACCATC
TCCTGCACTGGGAGCAGCTCCAACATCGGGGCAGGTTATGATGTACACTGGTACCAGCA
GCTTCCAGGAACAGCCCCCAAACTCCTCATCTATGGTAACAGCAATCGGCCCTCAGGGGT
CCCTGACCGATTCTCTGGCTCCAAGTCTGGCACCTCAGCCTCCCTGGCCATCACTGGGCT
CCAGGCTGAGGATGAGGCTGATTATTACTGCCAGTCCTATGACAGCAGCCTGAGTGGCCC
TGTGGTATTCGGCGGAGGGACCAAGCTGACCGTCCTA (SEQ ID NO: 498)
Her2_S6R3_CS_1B7
V_{H} with CDR1, CDR2 and CDR3 underlined
QVQLVQSGAEIKKPGESLKISCEGSGYRFTSHWIGWVRQMPGKGLEWMGIIYPGDSDTRYSP
SFQGQVTISADKSISTAYLQWSSLKASDTAMYYCARHSATHDAFDIWGRGTLVTVSS (SEQ ID NO: 499)
CAGGTGCAGCTGGTGCAGTCTGGGGCAGAAATAAAAAAGCCGGGGGAGTCTCTGAAGAT
CTCCTGTGAGGGTTCTGGATACAGGTTTACCAGCCACTGGATCGGCTGGGTGCGCCAGAT
GCCCGGGAAAGGCCTGGAGTGGATGGGGATCATCTATCCTGGTGACTCTGATACCAGAT
ACAGCCCGTCCTTCCAAGGCCAGGTCACCATCTCAGCCGACAAGTCCATCAGCACCGCCT
ACCTGCAGTGGAGCAGCCTGAAGGCCTCGGACACCGCCATGTATTACTGTGCGAGACAT
AGTGCGACGCATGATGCTTTTGATATCTGGGGCCGGGGCACCCTGGTCACCGTCTCGAGT (SEQ ID NO: 500)
V_{L} with CDR1, CDR2 and CDR3 underlined
QSVLTQPPSVSGAPGQRVTISCSGSSSNIGTGYDVHWYQQLPGTAPKLLIYSFNKRPSGVPDRF
SASKSGTSASLVITGLQAEDEADYYCQSYDNLSGPHVVFGTGTKLTVL (SEQ ID NO: 501)
CAGTCTGTGTTGACGCAGCCGCCCTCAGTGTCTGGGGCCCCAGGGCAGAGGGTCACCATC
TCCTGTAGTGGGAGCAGCTCCAACATCGGGACAGGTTACGATGTTCACTGGTACCAGCA
ACTTCCAGGAACAGCCCCCAAACTCCTCATCTATAGTTTCAATAAGCGGCCCTCAGGGGT
CCCTGACCGGTTCTCTGCCTCCAAGTCTGGCACCTCAGCCTCCCTGGTCATCACTGGGCTC
CAGGCTGAGGATGAGGCTGATTATTACTGCCAGTCCTATGACAATTTGAGTGGTCCCCAT
GTGGTTTTCGGCACAGGGACCAAGCTGACCGTCCTA (SEQ ID NO: 502)
Her2_S6R2_CS_1E5
V_{H} with CDR1, CDR2 and CDR3 underlined
EVQLVETGAEVKKPGASMKVSCKASGYSFTDYYMHWVRQAPGQGLEWMGWINPNSGDTN
YAQKFQGRVTMTRDTSITTAYMELSRLRSDDTAVYYCATERYNSGWEWGRGTLVTVSS (SEQ ID NO: 503)
GAGGTGCAGCTGGTGGAGACTGGGGCTGAGGTGAAGAAGCCTGGGGCCTCAATGAAGGT
CTCCTGCAAGGCTTCTGGATACAGCTTCACCGACTACTATATGCACTGGGTGCGACAGGC
CCCTGGACAAGGGCTTGAGTGGATGGGATGGATCAACCCTAATAGTGGTGACACAAACT
ATGCACAGAAGTTTCAGGGCAGGGTCACCATGACCAGGGACACGTCCATCACCACAGCC
TACATGGAGCTGAGCAGGCTGAGATCTGACGACACGGCCGTATATTACTGTGCGACAGA
GAGGTATAACAGTGGCTGGGAATGGGGCCGGGGCACCCTGGTCACCGTCTCGAGT (SEQ ID NO: 504)
V_{L} with CDR1, CDR2 and CDR3 underlined
QSALTQPASVSGSPGQSITISCTGTSSDVGAYNYVSWYQQHPGKAPKLMIYDVTTRPSGVSNR
FSGSKSGNTASLTISGLQAEDEADYYCTSYTRSSTVVFGGGTKLTVL (SEQ ID NO: 505)
CAGTCTGCCCTGACTCAGCCTGCCTCCGTGTCTGGGTCTCCTGGACAGTCGATCACCATCT
CCTGCACTGGAACCAGCAGTGACGTTGGTGCTTATAACTATGTCTCCTGGTACCAACAAC
ACCCAGGCAAAGCCCCCAAACTCATGATTTATGATGTCACTACTCGGCCCTCAGGGGTTT
CTAATCGCTTCTCTGGCTCCAAGTCTGGCAACACGGCCTCCCTGACCATCTCTGGGCTCC
AGGCTGAGGACGAGGCTGATTATTACTGCACCTCATATACTCGCAGCAGCACTGTGGTCT
TCGGCGGAGGGACCAAGCTGACCGTCCTA (SEQ ID NO: 506)
Her2_S6R3_BMV_1C2
V_{H} with CDR1, CDR2 and CDR3 underlined
KVQLVQSGSELKKPGASVKVSCQASGYTITNHSMNWVRQAPGQGLEWMGWINTNTGNPTY
AQGFTGRFVFSLDTSANTATLQITNVQAEDTAVYYCAREGSIDVSGTPYYYGMDAWGQGTT
VTVSS (SEQ ID NO: 507)
AAGGTGCAGCTGGTGCAGTCTGGGTCTGAGTTGAAGAAGCCTGGGGCCTCAGTGAAGGT
TTCCTGCCAGGCTTCTGGATACACCATCACTAACCATAGCATGAATTGGGTGCGACAGGC
CCCTGGGCAAGGGCTTGAGTGGATGGGATGGATCAACACCAACACTGGGAACCCTACGT
ATGCCCAGGGCTTCACAGGACGGTTTGTCTTCTCCTTGGACACCTCTGCCAACACGGCAA
CTTTGCAGATCACCAACGTGCAGGCTGAGGACACAGCCGTCTACTACTGTGCGAGAGAG
GGGAGTATAGACGTGTCTGGAACGCCCTACTACTACGGAATGGACGCCTGGGGGCAAGG
GACCACGGTCACCGTCTCCTCA (SEQ ID NO: 508)
V_{L} with CDR1, CDR2 and CDR3 underlined
QSVLTQPASVSGSPGQSITISCTGTSSDVGGYNYVSWYQQHPGKAPKLMIYEGSERPSGVPNR
FSGSKSGNTASLTISGLQAEDEADYYCSSYTTRSTRVFGGGTKLTIL (SEQ ID NO: 509)
CAGTCTGTGCTGACTCAGCCTGCCTCCGTGTCTGGGTCTCCTGGACAGTCGATCACCATCT
CCTGCACTGGAACCAGCAGTGACGTTGGTGGTTATAACTATGTCTCCTGGTACCAACAAC
ACCCAGGCAAAGCCCCCAAACTCATGATTTATGAGGGCAGTGAGCGGCCCTCAGGGGTT
CCTAATCGCTTCTCTGGCTCCAAGTCTGGCAACACGGCCTCCCTGACAATCTCTGGGCTC
CAGGCTGAGGACGAGGCTGATTATTACTGCAGCTCATATACAACCAGGAGCACTCGAGT
TTTCGGCGGAGGGACCAAGCTGACCATCCTA (SEQ ID NO: 510)
Her2_S5R2_DP47_1B10
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARVGAGENYYHYYIMDVWGRGTLVT
VSS (SEQ ID NO: 511)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGT
GGGGGCCGGGGAGAACTACTACCACTACTACATCATGGACGTCTGGGGCCGGGGCACCC
TGGTCACCGTCTCGAGT (SEQ ID NO: 512)
V_{L} with CDR1, CDR2 and CDR3 underlined
SSELTQDPAVSVALGQRVRVTCQGDSLRGYYASWYQQKPGQAPVLVIYGENNRPSGIPDRFS
GSSSGNTASLTIIGAQAEDEADYYCNSRHSSGNYLVFGGGTKLTVL (SEQ ID NO: 513)
TCTTCTGAGCTGACTCAGGACCCTGCTGTGTCTGTGGCCTTGGGACAGAGAGTCAGGGTC
ACATGCCAAGGAGACAGCCTCAGAGGCTATTATGCAAGCTGGTACCAGCAGAAGCCAGG
ACAGGCCCCTGTTCTTGTCATTTATGGTGAAAACAACCGGCCCTCAGGGATCCCAGACCG
ATTCTCTGGCTCCAGCTCAGGAAACACAGCTTCCTTGACCATCATTGGGGCTCAGGCGGA
AGATGAGGCTGACTATTACTGTAACTCCCGGCACAGCAGTGGTAATTATCTGGTGTTCGG
CGGAGGGACCAAGCTGACCGTCCTA (SEQ ID NO: 514)
Her2_S6R3_DP47_1C12
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARGFGDYWGRGTMVTVSS (SEQ ID NO: 515)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAGG
CTTTGGTGACTACTGGGGCCGGGGGACAATGGTCACCGTCTCGAGT (SEQ ID NO: 516)
V_{L} with CDR1, CDR2 and CDR3 underlined
SSELTQDPAVSVALGQTVRITCQGDSLRSYYASWYQQKPGQAPVLVIYAKNNRPSGIPDRFS
GSDSGNTASLTITGAQAEDEADYYCLSRDSSGNHLVFGGGTKLTVL (SEQ ID NO: 517)
TCTTCTGAGCTGACTCAGGACCCTGCTGTGTCTGTGGCCTTGGGACAGACAGTCAGGATC
ACATGCCAAGGAGACAGCCTCAGAAGTTATTATGCAAGCTGGTACCAGCAGAAGCCAGG
ACAGGCCCCTGTACTTGTCATCTATGCTAAAAACAACCGACCCTCAGGGATCCCAGACCG
ATTCTCTGGCTCCGACTCAGGAAACACAGCTTCCTTGACCATCACTGGGGCTCAGGCGGA
AGATGAGGCTGACTATTACTGTCTCTCCCGGGACAGCAGTGGTAACCATCTGGTATTCGG
CGGAGGGACCAAGCTGACCGTCCTA (SEQ ID NO: 518)
Her2_S5R2_DP47_1D10
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARTTADAFDIWGRGTMVTVSS (SEQ ID NO: 519)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAAC
TACGGCAGATGCTTTTGATATCTGGGGCAGAGGGACAATGGTCACCGTCTCGAGT (SEQ ID NO: 520)
V_{L} with CDR1, CDR2 and CDR3 underlined
SSELTQDPAVSVALGQTVKITCQGDSLRNYYAGWYQQKPGQAPVLVIYGENKRPSGIPDRFS
GSNSGNTASLTLTGAQAEDEADYYCNSRDSSSNLVVFGGGTKLTVL (SEQ ID NO: 521)
TCTTCTGAGCTGACTCAGGACCCTGCTGTGTCTGTGGCCTTGGGACAGACAGTCAAGATC
ACATGCCAAGGCGACAGCCTCAGAAACTATTATGCAGGCTGGTACCAGCAGAAGCCAGG
ACAGGCCCCTGTACTTGTCATCTATGGTGAAAACAAGCGGCCCTCAGGGATCCCTGACCG
ATTCTCTGGCTCCAACTCAGGAAACACAGCTTCCTTGACCCTCACTGGGGCTCAGGCGGA
AGATGAGGCTGACTATTACTGTAACTCCCGGGACAGCAGTAGTAACCTCGTGGTATTCGG
CGGAGGGACCAAGCTGACCGTCCTA (SEQ ID NO: 522)
Her2_S6R3_DP47_1H9
V_{H} with CDR1, CDR2 and CDR3 underlined
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCARTTADAFDIWGRGTTVTVSS (SEQ ID NO: 523)
GAGGTGCAGCTGTTGGAGTCTGGGGGAGGCTTGGTACAGCCTGGGGGGTCCCTGAGACT
CTCCTGTGCAGCCTCTGGATTCACCTTTAGCAGCTATGCCATGAGCTGGGTCCGCCAGGC
TCCAGGGAAGGGGCTGGAGTGGGTCTCAGCTATTAGTGGTAGTGGTGGTAGCACATACT
ACGCAGACTCCGTGAAGGGCCGGTTCACCATCTCCAGAGACAATTCCAAGAACACGCTG
TATCTGCAAATGAACAGCCTGAGAGCCGAGGACACGGCCGTGTATTACTGTGCGAGAAC
TACGGCAGATGCTTTTGATATCTGGGGGAGGGGGACCACGGTCACCGTCTCGAGT (SEQ ID NO: 524)
V_{L} with CDR1, CDR2 and CDR3 underlined
SSELTQDPAVSVALGQTVSITCQGDSLRNFYASWYLQKPGQAPILVIYGKNKRPSGIPDRVSG
SSSEDTASLTITGAQAEDEADYYCNSRDSSGNVVFGGGTKLTVL (SEQ ID NO: 525)
CTTCTGAGCTGACTCAGGACCCTGCTGTGTCTGTGGCCCTGGGACAGACAGTCAGCATCA
CATGCCAGGGAGACAGCCTCAGAAACTTTTATGCAAGCTGGTACCTGCAGAAGCCAGGA
CAGGCCCCAATACTTGTCATCTATGGTAAAAACAAGCGGCCCTCTGGGATCCCAGACCG
AGTCTCTGGCTCCAGCTCAGAAGACACAGCTTCCTTGACCATCACTGGGGCTCAGGCGGA
AGATGAGGCTGACTATTACTGTAACTCCCGGGACAGCAGTGGTAACGTGGTCTTCGGCG
GGGGGACCAAGCTGACCGTCCTA (SEQ ID NO: 526)
>HerSMIP_leader_CDS
atggaagcaccagcgcagcttctcttcctcctgctactctggctcccagataccaccggt (SEQ ID NO: 527)
>HerSMIP_Protein_leader
MEAPAQLLFLLLLWLPDTTG (SEQ ID NO: 528)
>HerSMIP_G4Sx3_linker_CDS
ggaggcggcggttcaggcggaggtggctctggcggtggcggaagt (SEQ ID NO: 529)
>HerSMIP_Protein_G4Sx3_linker
GGGGSGGGGSGGGGS (SEQ ID NO: 530)
>HerSMIP_SCCP_Hinge_CDS
gagcccaaatcttctgacaaaactcacacatgcccaccgtgccca (SEQ ID NO: 531)
>HerSMIP_Protein_SCCP_Hinge
EPKSSDKTHTCPPCP (SEQ ID NO: 532)
>Her101_CDS
atggaagcaccagcgcagcttctcttcctcctgctactctggctcccagataccaccggtgaagtgcagctggtgcagtctggggctgaggtgagg
aagcctggggcctcagtgaaggtttcctgcaggtcatctggatacaccttcaccacctactatttgcactggctacgacaggcccctggacaagggc
ttgagtggatgggagtaattaaccctagcggcggtgccacagcctacgcgcagagtttccagggcagagtcaccatgaccagggacacggctac
gagcacagtctatttggatctgagcagcctgagaactgaagacacggccgtgtattactgtgcgagatccaccccggcggagcagctcgtcccgg
gcttctggggcaaagggacaatggtcaccgtctcgagtggaggcggcggttcaggcggaggtggctctggcggtggcggaagtgcacttgaaat
tgtgatgacgcagtctccagccaccctgtctgtgtctccaggggacagagccaccctctcctgcagggccagtcagagtgttagcaccaacgtagc
ctggtaccagcagaaacctggccaacctcccaggctcctcatctatggtgcatccaccagggcctctggtgtcccagccaggttcagtggcagtgg
gtctgggacagagttcactctcaccatcagcagtctgcagtctgaagattttgcagtttattactgtcaacagtatggtgactggcctccgatcaccttc
ggccaagggacacgactggagattaaacgtgacgtacgcgagcccaaatcttctgacaaaactcacacatgcccaccgtgcccagcacctgaact
cctgggtggaccgtcagtcttcctcttccccccaaaacccaaggacaccctcatgatctcccggacccctgaggtcacatgcgtggtggtggacgt
gagccacgaagaccctgaggtcaagttcaactggtacgtggacggcgtggaggtgcataatgccaagacaaagccgcgggaggagcagtaca
acagcacgtaccgtgtggtcagcgtcctcaccgtcctgcaccaggactggctgaatggcaaggagtacaagtgcaaggtctccaacaaagccctc
ccagcccccatcgagaaaaccatctccaaagccaaagggcagccccgagaaccacaggtgtacaccctgcccccatcccgggatgagctgacc
aagaaccaggtcagcctgacctgcctggtcaaaggcttctatccaagcgacatcgccgtggagtgggagagcaatgggcagccggagaacaact
acaagaccacgcctcccgtgctggactccgacggctccttcttcctctacagcaagctcaccgtggacaagagcaggtggcagcaggggaacgt
cttctcatgctccgtgatgcatgaggctctgcacaaccactacacgcagaagagcctctccctgtctccgggtaaatga (SEQ ID NO: 533)
>Her101_Protein_leader-stop
MEAPAQLLFLLLLWLPDTTGEVQLVQSGAEVRKPGASVKVSCRSSGYTFTTYYLHWLRQAP
GQGLEWMGVINPSGGATAYAQSFQGRVTMTRDTATSTVYLDLSSLRTEDTAVYYCARSTPA
EQLVPGFWGKGTMVTVSSGGGGSGGGGSGGGGSALEIVMTQSPATLSVSPGDRATLSCRAS
QSVSTNVAWYQQKPGQPPRLLIYGASTRASGVPARFSGSGSGTEFTLTISSLQSEDFAVYYCQ
QYGDWPPITFGQGTRLEIKRDVREPKSSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 534)
>Her102_CDS
atggaagcaccagcgcagcttctcttcctcctgctactctggctcccagataccaccggtcagatgcagctggtgcagtctggggctgaggtgaag
aagccaggggcctcagtgaaagtctcctgcaagtcttctggatacaccttcaaggactactatatcaactgggtgcgacaggcccctggacaaggg
cttgagtgggtgggatggatcaaccctaaaaatggtatcacaaaatattcgcagaattttcagggcagggtctccatgaccacggatacgtccatca
gcacagtctacatggacctgagaggtctgacatctgacgacacggccgtttattattgtgcgagagacgcgaaccgccttagggtgggctggttcg
acccctggggccaaggaaccctggtcaccgtctcgagtggaggcggcggttcaggcggaggtggctctggcggtggcggaagtgcacaggct
gtgctgactcagccgtcctcagtgtctgggtccccagggcagagggtcagcatctcctgcactgggagcagctccaacatcggggcaggttatgat
gtacattggtatcaacaacttccaggaacagcccccaaactcctcatctacggtaacatcaatcggccctcaggggtccctgaccgattctctggctc
caagtctggcacctcagcctccctggccatcactgggctccaggctgaagatgaggctgattattactgccagtcctatgacagcagcctgagggct
gcggtattcggcggagggaccaaggtcaccgtcctaggtgacgtacgcgagcccaaatcttctgacaaaactcacacatgcccaccgtgcccag
cacctgaactcctgggtggaccgtcagtcttcctcttccccccaaaacccaaggacaccctcatgatctcccggacccctgaggtcacatgcgtggt
ggtggacgtgagccacgaagaccctgaggtcaagttcaactggtacgtggacggcgtggaggtgcataatgccaagacaaagccgcgggagg
agcagtacaacagcacgtaccgtgtggtcagcgtcctcaccgtcctgcaccaggactggctgaatggcaaggagtacaagtgcaaggtctccaac
aaagccctcccagcccccatcgagaaaaccatctccaaagccaaagggcagccccgagaaccacaggtgtacaccctgcccccatcccgggat
gagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagcagggg
aacgtcttctcatgctccgtgatgcatgaggctctgcacaaccactacacgcagaagagcctctccctgtctccgggtaaatga (SEQ ID NO: 535)
>Her102_Protein_leader-stop
MEAPAQLLFLLLLWLPDTTGQMQLVQSGAEVKKPGASVKVSCKSSGYTFKDYYINWVRQA
PGQGLEWVGWINPKNGITKYSQNFQGRVSMTTDTSISTVYMDLRGLTSDDTAVYYCARDAN
RLRVGWFDPWGQGTLVTVSSGGGGSGGGGSGGGGSAQAVLTQPSSVSGSPGQRVSISCTGSS
SNIGAGYDVHWYQQLPGTAPKLLIYGNINRPSGVPDRFSGSKSGTSASLAITGLQAEDEADYY
CQSYDSSLRAAVFGGGTKVTVLGDVREPKSSDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK. (SEQ ID NO: 536)
>Her103_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgcagggtactcatagcagtggctggtcctttgggt
actggggccagggcaccctggtcaccgtctcgagtggaggcggcggttcaggcggaggtggctctggcggtggcggaagtgcactgcctgtgc
tgactcagcccccctcagcgtctgggacccccgggcagagggtcaccatctcttgttctggaagcagctccaacatcggaagtaaaactgtaaact
ggtaccagcagctcccaggaacgacccccaaactcctcatctataggaataatcagcggccctcaggggtccctgaccgattctctggctccaagt
ctggcacctcagcctccctggccatcagtgggctccagtctgaggatgaggctgattattattgtgcagcatgggatgacagcctgaatggtctgata
ttcggcggagggaccaaggtcaccgtcctaggtgacgtacgcgagcccaaatcttctgacaaaactcacacatgcccaccgtgcccagcacctga
actcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 537)
>Her103_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAQGTHS
SGWSFGYWGQGTLVTVSSGGGGSGGGGSGGGGSALPVLTQPPSASGTPGQRVTISCSGSSSNI
GSKTVNWYQQLPGTTPKLLIYRNNQRPSGVPDRFSGSKSGTSASLAISGLQSEDEADYYCAA
WDDSLNGLIFGGGTKVTVLGDVREPKSSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQ
KSLSLSPGK. (SEQ ID NO: 538)
>Her104_CDS
atggaagcaccagcgcagcttctcttcctcctgctactctggctcccagataccaccggtcaggtacagctgcagcagtcaggggctgaggtgaag
aagcctggggcctcagtgaaggtctcctgcaaggtttccggatacaccccccctgatttatccatccactgggtgcgacaggctcctggagaaggg
cttgagtggatgggacgttttgattttgaagatggtgaaacaatcaacgcacagaagttccagggcagagtcaccatgaccgcggacacatccaca
aacacaggctacatggaggtgagcagcctgagatttgaggacacggccgtgtattactgtgcaacaacacttcgattttctggttactactacggtat
ggacttctggggccgaggaaccctggtcaccgtctcgagtggaggcggcggttcaggcggaggtggctctggcggtggcggaagtgcacagg
ctgtggtgatccaggagccatcgctctcagtgtcccctggagggacagtcacactcacttgtgccttgagctctggctcagtctccactggttactacc
ccagctggtaccagcagaccccaggccaggctccacgcacgctcatttacaacacagacactcgctcttctggggtccctggtcgcttctctggctc
catccttggggacaaagctgccctcaccatcacgggggcccaggcagatgatgaatctgattattactgtgtgctgtatatgggtagtggcatttggg
tgttcggcggagggaccaaggtcaccgtcctaggtgacgtacgcgagcccaaatcttctgacaaaactcacacatgcccaccgtgcccagcacct
gaactcctgggtggaccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcgggaggagca
gtacaacagcacgtaccgtgtggtcagcgtcctcaccgtcctgcaccaggactggctgaatggcaaggagtacaagtgcaaggtctccaacaaag
ccctcccagcccccatcgagaaaaccatctccaaagccaaagggcagccccgagaaccacaggtgtacaccctgcccccatcccgggatgagc
tgaccaagaaccaggtcagcctgacctgcctggtcaaaggcttctatccaagcgacatcgccgtggagtgggagagcaatgggcagccggaga
acaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 539)
>Her104_Protein_leader-stop
MEAPAQLLFLLLLWLPDTTGQVQLQQSGAEVKKPGASVKVSCKVSGYTPPDLSIHWVRQAP
GEGLEWMGRFDFEDGETINAQKFQGRVTMTADTSTNTGYMEVSSLRFEDTAVYYCATTLRF
SGYYYGMDFWGRGTLVTVSSGGGGSGGGGSGGGGSAQAVVIQEPSLSVSPGGTVTLTCALS
SGSVSTGYYPSWYQQTPGQAPRTLIYNTDTRSSGVPGRFSGSILGDKAALTITGAQADDESDY
YCVLYMGSGIWVFGGGTKVTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 540)
>Her105_CDS
atggaagcaccagcgcagcttctcttcctcctgctactctggctcccagataccaccggtcaggtccagctggtacagtctggagcagaggttaaaa
agcccggggagtctctgaagatctcctgtaagacttctggatacagctttaccagctattggatcggctgggtgcgccagatgcccgggaaaggcc
tggagtggatggggatcatctatcctggtgactctgataccagatacagcccgtcttttcaaggccaggtcaccatctcagccgacaagtccatcagc
accgcctacctgcagtggagcagcctgaaggcctcggacaccgccatgtattactgtgcgagacatgacctcccccatcaggagtatcaggacaa
cggtatggacgtctggggcaaaggaaccctggtcaccgtctcgagtggaggcggcggttcaggcggaggtggctctggcggtggcggaagtgc
acagactgtggtgatccaggagccatcgttctcagtgtcccctggagagacagtcacactcacttgtgccttgagctctggctcagtctctagtagtta
ctaccccagctggtaccagcagaccccaggccaggctccacgcgcgctcatctacaacacaaacactcgctcttctggggtccctgatcgcttctct
ggctccatccttgggaacaaagctgccctcaccatcacgggggcccaggcagatgatgaatctaactattactgtgcgctgtatctgggtagtggca
tttgggtgttcggcggagggaccaagctgaccgtcctaggtgacgtacgcgagcccaaatcttctgacaaaactcacacatgcccaccgtgccca
gcacctgaactcctgggtggaccgtcagtcttcctcttccccccaaaacccaaggacaccctcatgatctcccggacccctgaggtcacatgcgtg
gtggtggacgtgagccacgaagaccctgaggtcaagttcaactggtacgtggacggcgtggaggtgcataatgccaagacaaagccgcgggag
gagcagtacaacagcacgtaccgtgtggtcagcgtcctcaccgtcctgcaccaggactggctgaatggcaaggagtacaagtgcaaggtctccaa
caaagccctcccagcccccatcgagaaaaccatctccaaagccaaagggcagccccgagaaccacaggtgtacaccctgcccccatcccggga
tgagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagcagggg
aacgtcttctcatgctccgtgatgcatgaggctctgcacaaccactacacgcagaagagcctctccctgtctccgggtaaatga (SEQ ID NO: 541)
>Her105_Protein_leader-stop
MEAPAQLLFLLLLWLPDTTGQVQLVQSGAEVKKPGESLKISCKTSGYSFTSYWIGWVRQMP
GKGLEWMGIIYPGDSDTRYSPSFQGQVTISADKSISTAYLQWSSLKASDTAMYYCARHDLPH
QEYQDNGMDVWGKGTLVTVSSGGGGSGGGGSGGGGSAQTVVIQEPSFSVSPGETVTLTCAL
SSGSVSSSYYPSWYQQTPGQAPRALIYNTNTRSSGVPDRFSGSILGNKAALTITGAQADDESN
YYCALYLGSGIWVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 542)
>Her106_CDS
atggaagcaccagcgcagcttctcttcctcctgctactctggctcccagataccaccggtgaggtgcagctggtgcagtctggagcagaggtgaag
aagcccggggagtctctgaagatctcctgtcagggttctggatacacctttgccagccaatggatcgcctgggtgcgccagatgcccgggcaagg
cctggagtggatggggaccatctggcctggtgactctaatcccacatatagcccgtccttccaaggccaggtcaccatctcagccgacaagtccatc
agcaccgcctacctgcagtggagcagcctgaaggcctcggacaccgccatctattactgtgcgaggctctacaataactatccctacttctacggtat
ggacgtctggggccaggggacaatggtcaccgtctcgagtggaggcggcggttcaggcggaggtggctctggcggtggcggaagtgcacaga
ctgtggtgctccaggagccatcgttctcagtgtcccctggagggacagtcacactcacttgtggcttgaggtctggctcagtctctactacttactacc
ccagctggtaccagcagaccccaggccaggctccacgcacgctcatctacagcacaaacactcgctcttctggggtccctgatcgcttctctggct
ccatcgtcgggaacaaagctgccctcaccatcacgggggcccaggcagatgatgaatctgattattactgtgcgctatacctgggtagtggcacttg
ggtgttcggcggagggaccaagctgaccgtcctaggtgacgtacgcgagcccaaatcttctgacaaaactcacacatgcccaccgtgcccagca
cctgaactcctgggtggaccgtcagtcttcctcttccccccaaaacccaaggacaccctcatgatctcccggacccctgaggtcacatgcgtggtgg
tggacgtgagccacgaagaccctgaggtcaagttcaactggtacgtggacggcgtggaggtgcataatgccaagacaaagccgcgggaggagc
agtacaacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccgggatgag
ctgaccaagaaccaggtcagcctgacctgcctggtcaaaggcttctatccaagcgacatcgccgtggagtgggagagcaatgggcagccggag
aacaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 543)
>Her106_Protein_leader-stop
MEAPAQLLFLLLLWLPDTTGEVQLVQSGAEVKKPGESLKISCQGSGYTFASQWIAWVRQMP
GQGLEWMGTIWPGDSNPTYSPSFQGQVTISADKSISTAYLQWSSLKASDTAIYYCARLYNNY
PYFYGMDVWGQGTMVTVSSGGGGSGGGGSGGGGSAQTVVLQEPSFSVSPGGTVTLTCGLR
SGSVSTTYYPSWYQQTPGQAPRTLIYSTNTRSSGVPDRFSGSIVGNKAALTITGAQADDESDY
YCALYLGSGTWVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 544)
>Her107_CDS
atggaagcaccagcgcagcttctcttcctcctgctactctggctcccagataccaccggtcagatgcagctggtgcagtctggggctgaggtgaag
aagcctggggcctcagtgaaggtttcctgcaaggcatctggatacaccttcaccagctactatatacactgggtgcgacaggcccctggacagggc
cctgagtggatgggaataatcctccctagtggtggcagcaccagctacgcacaggagttccagggcagactctccatgaccagggacacgtccac
gagcacagtgtacatggagctgagcgacctgagatctgacgacacggccatttattattgtgcgagagactatgataggagtgcttatcttgatatctg
gggccgagggacaatggtcaccgtctcgagtggaggcggcggttcaggcggaggtggctctggcggtggcggaagtgcacagtctgtgttgac
gcagccgccctcagcgtctgggacccccgggcagagggtcaccatctcttgttctggaagcagctccaacatcggaagaaatactgtaaactggt
acaagcagttcccaggaacggcccccaaactcctcatctatagtgataataagcggccctcagggatccctgaccgattctctggctccaagtctgg
cacctcagcctccctggccatcagtgggctccagtctggggatgaggctgattattactgtgccgcatgggatgacagcctgaatggccatgtggta
ttcggcggagggaccaagctgaccgtcctaggtgacgtacgcgagcccaaatcttctgacaaaactcacacatgcccaccgtgcccagcacctga
actcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 545)
>Her107_Protein_leader-stop
MEAPAQLLFLLLLWLPDTTGQMQLVQSGAEVKKPGASVKVSCKASGYTFTSYYIHWVRQAP
GQGPEWMGIILPSGGSTSYAQEFQGRLSMTRDTSTSTVYMELSDLRSDDTAIYYCARDYDRS
AYLDIWGRGTMVTVSSGGGGSGGGGSGGGGSAQSVLTQPPSASGTPGQRVTISCSGSSSNIG
RNTVNWYKQFPGTAPKLLIYSDNKRPSGIPDRFSGSKSGTSASLAISGLQSGDEADYYCAAW
DDSLNGHVVFGGGTKLTVLGDVREPKSSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQ
KSLSLSPGK. (SEQ ID NO: 546)
>Her108_CDS
atggaagcaccagcgcagcttctcttcctcctgctactctggctcccagataccaccggtgaagtgcagctggtgcagtctggggctgaggtgaag
aagcctggggcctcagtgagggtctcctgcaagggttctggaaacaccttcaccggccactacatccactgggtgcgacaggcccctggacaag
gacttgagtggctgggatggatcgaccctaacactggtgacatacagtattcagaaaactttaagggctcggtcaccttgaccagggacccatccat
caactcagtcttcatggacctgatcaggctgacatctgacgacacggccatgtattactgtgcgagagaaggtgccgggctcgccaactactattact
acggtctggacgtctggggccgagggacaatggtcaccgtctcgagtggaggcggcggttcaggcggaggtggctctggcggtggcggaagt
gcacagactgtggtgctccaggagccttcgttctcagtgtcccctggggggacagtcacactcacttgtggcttgaactttggctcagtctctactgct
tactaccccagttggtaccagcagaccccaggccaagctccacgcacgctcatctacggcacaaatattcgttcctctggggtcccggatcgcttct
ctggctccatcgtagggaacaaagctgccctcaccatcacgggggcccagacagaagatgagtctgattattattgtgcgctgtatatgggtagtgg
catgctcttcggcggcgggaccaaggtcaccgtcctaggtgacgtacgcgagcccaaatcttctgacaaaactcacacatgcccaccgtgcccag
cacctgaactcctgggtggaccgtcagtcttcctcttccccccaaaacccaaggacaccctcatgatctcccggacccctgaggtcacatgcgtggt
ggtggacgtgagccacgaagaccctgaggtcaagttcaactggtacgtggacggcgtggaggtgcataatgccaagacaaagccgcgggagg
agcagtacaacagcacgtaccgtgtggtcagcgtcctcaccgtcctgcaccaggactggctgaatggcaaggagtacaagtgcaaggtctccaac
aaagccctcccagcccccatcgagaaaaccatctccaaagccaaagggcagccccgagaaccacaggtgtacaccctgcccccatcccgggat
gagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagcagggg
aacgtcttctcatgctccgtgatgcatgaggctctgcacaaccactacacgcagaagagcctctccctgtctccgggtaaatga (SEQ ID NO: 547)
>Her108_Protein_leader-stop
MEAPAQLLFLLLLWLPDTTGEVQLVQSGAEVKKPGASVRVSCKGSGNTFTGHYIHWVRQAP
GQGLEWLGWIDPNTGDIQYSENFKGSVTLTRDPSINSVFMDLIRLTSDDTAMYYCAREGAGL
ANYYYYGLDVWGRGTMVTVSSGGGGSGGGGSGGGGSAQTVVLQEPSFSVSPGGTVTLTCG
LNFGSVSTAYYPSWYQQTPGQAPRTLIYGTNIRSSGVPDRFSGSIVGNKAALTITGAQTEDES
DYYCALYMGSGMLFGGGTKVTVLGDVREPKSSDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 548)
>Her109_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgccagagatttaactggcccaaaccactggttcttcga
tctctgggggcgggggaccacggtcaccgtctcgagtggaggcggcggttcaggcggaggtggctctggcggtggcggaagtgcacagtctgt
gctgactcagccaccctccgtgtccgtgtccccaggacagacagccagcatctcctgctctggacatagattgggcgataagtatgttagttggtatc
ggcagaggccgggccagtcccctgtgctggtcatctatcaagatgagaagaggtactcagggatctctgagcgattctctggctccaactctggga
acgtagccactctgaccatcaccgggacccaggctatggatgaggctgactatcactgtcaggcgtgggacagcaccactgtggtgttcggcgga
gggaccaagctgaccgtcctaggtgacgtacgcgagcccaaatcttctgacaaaactcacacatgcccaccgtgcccagcacctgaactcctggg
tggaccgtcagtcttcctcttccccccaaaacccaaggacaccctcatgatctcccggacccctgaggtcacatgcgtggtggtggacgtgagcca
cgaagaccctgaggtcaagttcaactggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgcaaggtctccaacaaagccctcccagccc
ccatcgagaaaaccatctccaaagccaaagggcagccccgagaaccacaggtgtacaccctgcccccatcccgggatgagctgaccaagaacc
aggtcagcctgacctgcctggtcaaaggcttctatccaagcgacatcgccgtggagtgggagagcaatgggcagccggagaacaactacaagac
cacgcctcccgtgctggactccgacggctccttcttcctctacagcaagctcaccgtggacaagagcaggtggcagcaggggaacgtcttctcatg
ctccgtgatgcatgaggctctgcacaaccactacacgcagaagagcctctccctgtctccgggtaaatga (SEQ ID NO: 549)
>Her109_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARDLTG
PNHWFFDLWGRGTTVTVSSGGGGSGGGGSGGGGSAQSVLTQPPSVSVSPGQTASISCSGHRL
GDKYVSWYRQRPGQSPVLVIYQDEKRYSGISERFSGSNSGNVATLTITGTQAMDEADYHCQ
AWDSTTVVFGGGTKLTVLGDVREPKSSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 550)
>Her110_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagaggcagtagagtggggacgatttggggaag
ccttgacgactggggcaaagggacaatggtcaccgtctcgagtggaggcggcggttcaggcggaggtggctctggcggtggcggaagtgcact
tgaaacgacactcacgcagtctccaggcaccctgtctttgtctccaggggaaagagccaccctctcctgcagggccagtcagagtagtagcagca
gctacttagcctggtaccagcagaaacctggccaggctcccaggctcctcatctatgctgcatccagcagggccactggcgtcccagacaggttca
gtggcagtgggtctgggacagacttcactctcaccatcagcagactggagcctgaagattttgcagtgtattactgtcagcagtatggtagctcacgg
ttcaccttcggccaagggacacgactggagattaaacgtgacgtacgcgagcccaaatcttctgacaaaactcacacatgcccaccgtgcccagc
acctgaactcctgggtggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaagccgcgggagga
gcagtacaacagcacgtaccgtgtggtcagcgtcctcaccgtcctgcaccaggactggctgaatggcaaggagtacaagtgcaaggtctccaaca
aagccctcccagcccccatcgagaaaaccatctccaaagccaaagggcagccccgagaaccacaggtgtacaccctgcccccatcccgggatg
agctgaccaagaaccaggtcagcctgacctgcctggtcaaaggcttctatccaagcgacatcgccgtggagtgggagagcaatgggcagccgg
agaacaactacaagaccacgcctcccgtgctggactccgacggctccttcttcctctacagcaagctcaccgtggacaagagcaggtggcagcagggg
aacgtcttctcatgctccgtgatgcatgaggctctgcacaaccactacacgcagaagagcctctccctgtctccgggtaaatga (SEQ ID NO: 551)
>Her110_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARGSRV
GTIWGSLDDWGKGTMVTVSSGGGGSGGGGSGGGGSALETTLTQSPGTLSLSPGERATLSCR
ASQSSSSSYLAWYQQKPGQAPRLLIYAASSRATGVPDRFSGSGSGTDFTLTISRLEPEDFAVY
YCQQYGSSRFTFGQGTRLEIKRDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 552)
>Her111_CDS
atggaagcaccagcgcagcttctcttcctcctgctactctggctcccagataccaccggtcaggtgcagctggtgcagtctggagcagaggtgaaa
aagcccggggagtctctgaagatctcctgtaagggttctggatacagttttgccaactacgggataggctgggtgcgccagatgcccgggaaagg
cctggagtggatgggggtcatctatcctggtgactctgatatcagatacagcccgtccttccaaggccaggtcatcttctcagccgacaggtccatca
gcaccgcctacctgcagtggagcagcctgaaggcctcggacaccgccatgtattattgtgcgagacatctctcgtggctggtcggggggaactac
ggtatggacgtctggggcaaagggacaatggtcaccgtctcgagtggaggcggcggttcaggcggaggtggctctggcggtggcggaagtgca
cagactgtggtgatccaggagccatcgttctcagtgtcccctggagggacagtcacactcacttgtggcttgacctctggctcagtctctactagtcac
taccccagctggtaccagcagaccccaggccaggctccacgcacgctcatctacagcacaaacactcgctcttctggggtccctggtcgcttctct
ggctccatccttgggaacaaagctgccctcaccatcacgggggcccaggcagacgatgaatctgattattattgtgtgctgtatatgggtggtggcat
ttcggtgttcggcggagggaccaaggtcaccgtcctaggtgacgtacgcgagcccaaatcttctgacaaaactcacacatgcccaccgtgcccag
cacctgaactcctgggtggaccgtcagtcttcctcttccccccaaaacccaaggacaccctcatgatctcccggacccctgaggtcacatgcgtggt
ggtggacgtgagccacgaagaccctgaggtcaagttcaactggtacgtggacggcgtggaggtgcataatgccaagacaaagccgcgggagg
agcagtacaacagcacgtaccgtgtggtcagcgtcctcaccgtcctgcaccaggactggctgaatggcaaggagtacaagtgcaaggtctccaac
aaagccctcccagcccccatcgagaaaaccatctccaaagccaaagggcagccccgagaaccacaggtgtacaccctgcccccatcccgggat
gagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagcagggg
aacgtcttctcatgctccgtgatgcatgaggctctgcacaaccactacacgcagaagagcctctccctgtctccgggtaaatga (SEQ ID NO: 553)
>Her111_Protein_leader-stop
MEAPAQLLFLLLLWLPDTTGQVQLVQSGAEVKKPGESLKISCKGSGYSFANYGIGWVRQMP
GKGLEWMGVIYPGDSDIRYSPSFQGQVIFSADRSISTAYLQWSSLKASDTAMYYCARHLSWL
VGGNYGMDVWGKGTMVTVSSGGGGSGGGGSGGGGSAQTVVIQEPSFSVSPGGTVTLTCGL
TSGSVSTSHYPSWYQQTPGQAPRTLIYSTNTRSSGVPGRFSGSILGNKAALTITGAQADDESD
YYCVLYMGGGISVFGGGTKVTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 554)
>Her112_CDS
atggaagcaccagcgcagcttctcttcctcctgctactctggctcccagataccaccggtgaggtgcagctggtgcagtctggggcagaggtgaaa
cagcccggggagtctctgaagatctcctgtaagggttctggatacagctttagcaactactggatcggctgggtgcgccagatgcccgggaaagg
cctggagtggatggggatcatctatcctgatgactctgataccagatacagcccgtccttccaaggccaggtcaccatctcagccgacaggtccatc
agcaccgcctacctgcagtggagcagcctgaaggcctcggacaccgccacgtattactgtgcgagaggaaatgttataaatggaaataccaatgct
tttgatatctgggggcgggggaccacggtcaccgtctcgagtggaggcggcggttcaggcggaggtggctctggcggtggcggaagtgcacag
gctgtggtgatccaggagccatcgttctcagtgtcccctggagggacagtcacactcacttgtggcttgagctctggctcagtctctactggttactac
cccagctggtaccagcagaccccaggccaggctccacgcacgctcatctacaacacaaacagtcgctcttctggggtccctgatcgcttctctggc
tccatccttgggaacaaagctgccctcaccatcacgggggcccaggcagatgatgaatctgattattactgtgtgctgtatatgggtagtggcatttcg
gtattcggcggagggaccaagctgaccgtcctaggtgacgtacgcgagcccaaatcttctgacaaaactcacacatgcccaccgtgcccagcacc
tgaactcctgggtggaccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcgggaggagca
gtacaacagcacgtaccgtgtggtcagcgtcctcaccgtcctgcaccaggactggctgaatggcaaggagtacaagtgcaaggtctccaacaaag
ccctcccagcccccatcgagaaaaccatctccaaagccaaagggcagccccgagaaccacaggtgtacaccctgcccccatcccgggatgagc
tgaccaagaaccaggtcagcctgacctgcctggtcaaaggcttctatccaagcgacatcgccgtggagtgggagagcaatgggcagccggaga
acaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 555)
>Her112_Protein_leader-stop
MEAPAQLLFLLLLWLPDTTGEVQLVQSGAEVKQPGESLKISCKGSGYSFSNYWIGWVRQMP
GKGLEWMGIIYPDDSDTRYSPSFQGQVTISADRSISTAYLQWSSLKASDTATYYCARGNVING
NTNAFDIWGRGTTVTVSSGGGGSGGGGSGGGGSAQAVVIQEPSFSVSPGGTVTLTCGLSSGS
VSTGYYPSWYQQTPGQAPRTLIYNTNSRSSGVPDRFSGSILGNKAALTITGAQADDESDYYC
VLYMGSGISVFGGGTKLTVLGDVREPKSSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQ
KSLSLSPGK. (SEQ ID NO: 556)
>Her113_CDS
atggaagcaccagcgcagcttctcttcctcctgctactctggctcccagataccaccggtgaggtgctgctgttggagtctgggggaggcttggtac
agcctggggggtccctgagactctcctgtgcagcctctggattcacctttagcagctatgccatgagctgggtccgccaggctccagggaagggg
ctggagtgggtctcagctatcagtggtagtggtggtagcacatactacgcagactccgcgaagggccggttcaccatctccagagacaattccaag
aacacgctgtatctgcaaatgaacagcctgagagccgaggacacggccgtgtattactgtgcgagggacagggtctctaactggaactactacgg
ccaggacagctactttgactactggggccaagggacaatggtcaccgtctcgagtggaggcggcggttcaggcggaggtggctctggcggtggc
ggaagtgcacaggctgtgctgactcagccaccctcagcgtctgggacccccgggcagagggtcaccatctcttgttctggaagcagctccaacat
cggaagtaattatgtatactggtaccagcaactcccaggaacggcccccaaagtcctcatctataggaataatcagcggccctcaggggtccctga
ccgattctctggctccaagtctggcacctcagcgtccctggccatcagtgggctccggtccgaggatgaggctgattattactgtgcatcatgggatg
gcagcctgagtggtccggtgttcggcggagggaccaagctgaccgtcctaggtgacgtacgcgagcccaaatcttctgacaaaactcacacatgc
ccaccgtgcccagcacctgaactcctgggtggaccgtcagtcttcctcttccccccaaaacccaaggacaccctcatgatctcccggacccctgag
gtcacatgcgtggtggtggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatggcaaggagtacaagt
gcaaggtctccaacaaagccctcccagcccccatcgagaaaaccatctccaaagccaaagggcagccccgagaaccacaggtgtacaccctgc
ccccatcccgggatgagctgaccaagaaccaggtcagcctgacctgcctggtcaaaggcttctatccaagcgacatcgccgtggagtgggagag
caatgggcagccggagaacaactacaagaccacgcctcccgtgctggactccgacggctccttcttcctctacagcaagctcaccgtggacaaga
gcaggtggcagcaggggaacgtcttctcatgctccgtgatgcatgaggctctgcacaaccactacacgcagaagagcctctccctgtctccgggta
aatga (SEQ ID NO: 557)
>Her113_Protein_leader-stop
MEAPAQLLFLLLLWLPDTTGEVLLLESGGGLVQPGGSLRLSCAASGFTFSSYAMSWVRQAPG
KGLEWVSAISGSGGSTYYADSAKGRFTISRDNSKNTLYLQMNSLRAEDTAVYYCARDRVSN
WNYYGQDSYFDYWGQGTMVTVSSGGGGSGGGGSGGGGSAQAVLTQPPSASGTPGQRVTIS
CSGSSSNIGSNYVYWYQQLPGTAPKVLIYRNNQRPSGVPDRFSGSKSGTSASLAISGLRSEDE
ADYYCASWDGSLSGPVFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK. (SEQ ID NO: 558)
>Her114_CDS
atggaagcaccagcgcagcttctcttcctcctgctactctggctcccagataccaccggtgaggtgcagctggtgcagtctgggggaggcttggta
cagcctgggggatccctgagactctcctgtgcagcctctggattcacctttagcagctatcccatgcactgggtccgccaggctccaggcaagggg
ctggagtgggtggcagttgtatcgttcgatggatctaagaaatactctgcagactccgtgaagggccgattcaccatctccagagacatctccaaga
acacgctgtatctgcaaatgaacagcctgagagctgaggacacggctgtatattactgtgcgaaagatcgctatgattcggggactttctactacggc
atggacgtctggggccggggcaccctggtcaccgtctcgagtggtggaggcggttcaggcggaggtggcagcggcggtggcggatcgcagtct
gccctgactcagcctgcctccgtgtctggatctcgtggacagtcgatcaccatctcctgcactggaaccactggtgacgttggtggttatgactatgtc
tcctggtaccaacagcacccaggcagagcccccaaactcctcatctatggtaacagcaatcggccctcaggggtccctgatcgcttctctgcctcca
agtccggcaatacggcctccctgaccatctctggactccaggctgaggatgaggctgattatttctgcagcacatatgcaccccccggtattattatgt
tcggcggagggaccaagctgaccgtcctaggtgacgtacgcgagcccaaatcttctgacaaaactcacacatgcccaccgtgcccagcacctga
actcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 559)
>Her114_Protein_leader-stop
MEAPAQLLFLLLLWLPDTTGEVQLVQSGGGLVQPGGSLRLSCAASGFTFSSYPMHWVRQAP
GKGLEWVAVVSFDGSKKYSADSVKGRFTISRDISKNTLYLQMNSLRAEDTAVYYCAKDRYD
SGTFYYGMDVWGRGTLVTVSSGGGGSGGGGSGGGGSQSALTQPASVSGSRGQSITISCTGTT
GDVGGYDYVSWYQQHPGRAPKLLIYGNSNRPSGVPDRFSASKSGNTASLTISGLQAEDEADY
FCSTYAPPGIIMFGGGTKLTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 560)
>Her115_CDS
atggaagcaccagcgcagcttctcttcctcctgctactctggctcccagataccaccggtgaggtgcagctggtggagtctgggggaggcttggta
cagcctggggggtccctgagactctcctgtgctgcctctggattcacctttgacagctatgccatgagctgggtccgccaggctccagggaagggg
ctggagtgggtctcaattattagtggtagagatggttacacatactacacagactccgtgaagggtcggttcaccatctccagagacaattccaagaa
cacggtgtatctgcaaatgaacagcctgagagccgaggacacgggtgtgtattattgtgcgagaaatggggagtggcccggaatcttagactactg
gggcagggggacaatggtcaccgtctcctcaggtggaggcggttcaggcggaggtggcagcggcggtggcggatcggacatccagatgaccc
agtctccttccaccctgtctgcatctattggagacagagtcaccatcacctgccgggccagtgagggtatttatcactggttggcctggtatcagcaga
agccagggaaagcccctaaactcctgatctataaggcctctagtttagccagtggggccccatcaaggttcagcggcagtggatctgggacagatt
tcactctcaccatcagcagcctgcagcctgatgattttgcaacttattactgccaacaatatagtaattatccgctcactttcggcggagggaccaagct
ggagatcaaacgtgacgtacgcgagcccaaatcttctgacaaaactcacacatgcccaccgtgcccagcacctgaactcctgggtggaccgtcag
tcttcctcttccccccaaaacccaaggacaccctcatgatctcccggacccctgaggtcacatgcgtggtggtggacgtgagccacgaagaccctg
aggtcaagttcaactggtacgtggacggcgtggaggtgcataatgccaagacaaagccgcgggaggagcagtacaacagcacgtaccgtgtgg
tcagcgtcctcaccgtcctgcaccaggactggctgaatggcaaggagtacaagtgcaaggtctccaacaaagccctcccagcccccatcgagaa
aaccatctccaaagccaaagggcagccccgagaaccacaggtgtacaccctgcccccatcccgggatgagctgaccaagaaccaggtcagcct
gacctgcctggtcaaaggcttctatccaagcgacatcgccgtggagtgggagagcaatgggcagccggagaacaactacaagaccacgcctcc
cgtgctggactccgacggctccttcttcctctacagcaagctcaccgtggacaagagcaggtggcagcaggggaacgtcttctcatgctccgtgat
gcatgaggctctgcacaaccactacacgcagaagagcctctccctgtctccgggtaaatga (SEQ ID NO: 561)
>Her115_Protein_leader-stop
MEAPAQLLFLLLLWLPDTTGEVQLVESGGGLVQPGGSLRLSCAASGFTFDSYAMSWVRQAP
GKGLEWVSIISGRDGYTYYTDSVKGRFTISRDNSKNTVYLQMNSLRAEDTGVYYCARNGEW
PGILDYWGRGTMVTVSSGGGGSGGGGSGGGGSDIQMTQSPSTLSASIGDRVTITCRASEGIYH
WLAWYQQKPGKAPKLLIYKASSLASGAPSRFSGSGSGTDFTLTISSLQPDDFATYYCQQYSN
YPLTFGGGTKLEIKRDVREPKSSDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLS
PGK. (SEQ ID NO: 562)
>Her116_CDS
atggaagcaccagcgcagcttctcttcctcctgctactctggctcccagataccaccggtcaggtgcagctggtggagtctgggggaggcgtggtc
cagcctgggaggtccctgagactctcctgtgcagcctctggattcaccttcagtagctatgctatgaactgggtccgccaggctccaggcaagggg
ctggagtgggtggcaactatatcatatgatggaagcaataaatactacgcagactccgtgaagggccgattcaccatctccagagacaattccaaga
acacgctgtatctgcaaatgaacagcctgagagccgaggacacggctgtgtattactgtgcgagaccggccccgtatagcagctccggcgcttttg
atatctggggccaaggcaccctggtcaccgtctcttcaggtggaggcggttcaggcggaggtggcagcggcggtggcggatcggacatccagat
gacccagtctccttccaccctgtctgcatctattggagacagagtcaccatcacctgccgggccagtgagggtatttatcactggttggcctggtatca
gcagaagccagggaaagcccctaaactcctgacctataaggcctctagtttagccagtggggccccatcaaggttcagcggcagtggatctggga
cagatttcactctcaccatcagcagcctgcagcctgatgattttgcaacttattactgccaacaatatagtaattatccgctcactttcggcggagggac
caagctggagatcaaacgtgacgtacgcgagcccaaatcttctgacaaaactcacacatgcccaccgtgcccagcacctgaactcctgggtggac
cgtcagtcttcctcttccccccaaaacccaaggacaccctcatgatctcccggacccctgaggtcacatgcgtggtggtggacgtgagccacgaag
accctgaggtcaagttcaactggtacgtggacggcgtggaggtgcataatgccaagacaaagccgcgggaggagcagtacaacagcacgtacc
gtgtggtcagcgtcctcaccgtcctgcaccaggactggctgaatggcaaggagtacaagtgcaaggtctccaacaaagccctcccagcccccatc
gagaaaaccatctccaaagccaaagggcagccccgagaaccacaggtgtacaccctgcccccatcccgggatgagctgaccaagaaccaggtc
agcctgacctgcctggtcaaaggcttctatccaagcgacatcgccgtggagtgggagagcaatgggcagccggagaacaactacaagaccacg
cctcccgtgctggactccgacggctccttcttcctctacagcaagctcaccgtggacaagagcaggtggcagcaggggaacgtcttctcatgctcc
gtgatgcatgaggctctgcacaaccactacacgcagaagagcctctccctgtctccgggtaaatga (SEQ ID NO: 563)
>Her116_Protein_leader-stop
MEAPAQLLFLLLLWLPDTTGQVQLVESGGGVVQPGRSLRLSCAASGFTFSSYAMNWVRQAP
GKGLEWVATISYDGSNKYYADSVKGRFTISRDNSKNTLYLQMNSLRAEDTAVYYCARPAPY
SSSGAFDIWGQGTLVTVSSGGGGSGGGGSGGGGSDIQMTQSPSTLSASIGDRVTITCRASEGIY
HWLAWYQQKPGKAPKLLTYKASSLASGAPSRFSGSGSGTDFTLTISSLQPDDFATYYCQQYS
NYPLTFGGGTKLEIKRDVREPKSSDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSL
SPGK. (SEQ ID NO: 564)
>Her117_CDS
atggaagcaccagcgcagcttctcttcctcctgctactctggctcccagataccaccggtcagatgcagctggtgcagtctggggcagaggtgaaa
aagcccggggagtctctgaagatgtcctgtaagggttctggatacagctttaccagctactggatcggctgggtgcgccagatgcccgggaaagg
cctggagtggatggggatcatctatcctggtaactccgataccagatacaacccgtccttcgaaggccaggtcaccatctcagccgacaagtccatc
aacaccgccttcctgcagtggaacagcctgaaggcctcggacaccgccatatattattgtgcgcgggctccctgggtgggtgcttttgatacttggg
gccaggggacaatggtcaccgtctcttcaggtggaggcggttcaggcggaggtggcagcggcggtggcggatcggacatcgtgatgacccagt
ctccttccaccctgtctgcatctgtaggagacagagtcaccatcacttgccgggccagtcagggtattagtagctggttggcctggtatcagcagaaa
ccagggagagcccctaaggtcttgatctataaggcatctactttagaaagtggggtcccatcaaggttcagcggcagtggatctgggacagatttca
ctctcaccatcagcagtctgcaacctgaagattttgcaacttactactgtcaacagagttacagtaccccgtggacgttcggccaagggaccaagctg
gagatcaaacgtgacgtacgcgagcccaaatcttctgacaaaactcacacatgcccaccgtgcccagcacctgaactcctgggtggaccgtcagt
cttcctcttccccccaaaacccaaggacaccctcatgatctcccggacccctgaggtcacatgcgtggtggtggacgtgagccacgaagaccctga
ggtcaagttcaactggtacgtggacggcgtggaggtgcataatgccaagacaaagccgcgggaggagcagtacaacagcacgtaccgtgtggt
cagcgtcctcaccgtcctgcaccaggactggctgaatggcaaggagtacaagtgcaaggtctccaacaaagccctcccagcccccatcgagaaa
accatctccaaagccaaagggcagccccgagaaccacaggtgtacaccctgcccccatcccgggatgagctgaccaagaaccaggtcagcctg
acctgcctggtcaaaggcttctatccaagcgacatcgccgtggagtgggagagcaatgggcagccggagaacaactacaagaccacgcctccc
gtgctggactccgacggctccttcttcctctacagcaagctcaccgtggacaagagcaggtggcagcaggggaacgtcttctcatgctccgtgatg
catgaggctctgcacaaccactacacgcagaagagcctctccctgtctccgggtaaatga (SEQ ID NO: 565)
>Her117_Protein_leader-stop
MEAPAQLLFLLLLWLPDTTGQMQLVQSGAEVKKPGESLKMSCKGSGYSFTSYWIGWVRQM
PGKGLEWMGIIYPGNSDTRYNPSFEGQVTISADKSINTAFLQWNSLKASDTAIYYCARAPWV
GAFDTWGQGTMVTVSSGGGGSGGGGSGGGGSDIVMTQSPSTLSASVGDRVTITCRASQGISS
WLAWYQQKPGRAPKVLIYKASTLESGVPSRFSGSGSGTDFTLTISSLQPEDFATYYCQQSYST
PWTFGQGTKLEIKRDVREPKSSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSP
GK. (SEQ ID NO: 566)
>Her118_CDS
atggaagcaccagcgcagcttctcttcctcctgctactctggctcccagataccaccggtcaggtgcagctgcaggagtcgggcccaggactggtg
gagccttcggagaccctgtccctcacctgcagtgtctctggcggctccatcagcagcagtagttcctcttggggctggatccgccagcccccaggg
aaggggctggagtggattgggagtatctattacagtggagaaacctattataatccgtccctcaagaggcgtgtcaccatatccacagacacgtcca
agaaccagttgtccctggagctggcctctgtgaccgccgcagacacggctgtatattactgtgcgaggcaagtcaccagttatggttctgactacttt
gactactggggcaaaggaaccctggtcaccgtctcgagtggaggcggcggttcaggcggaggtggctctggcggtggcggaagtgcacaggct
gtggtgatccaggagccatcgttctcagtgtcccctggagggacagtcacacttacttgtggcttgagctctggctcagtctcttctaattactacccca
gctggtaccagcagaccccaggccagactccacgcacgctcatctacaacacaaacactcgctcttctggggtccctgatcgcttctctggctccat
ccttgggaacaaagctgccctcaccatcacgggggcccaggcagatgatgaatctgattattactgtgtgttgtatatgggtagtggcattcgcgtgtt
cggcggagggaccaaggtcaccgtcctaggtgacgtacgcgagcccaaatcttctgacaaaactcacacatgcccaccgtgcccagcacctgaa
ctcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 567)
>Her118_Protein_leader-stop
MEAPAQLLFLLLLWLPDTTGQVQLQESGPGLVEPSETLSLTCSVSGGSISSSSSSWGWIRQPPG
KGLEWIGSIYYSGETYYNPSLKRRVTISTDTSKNQLSLELASVTAADTAVYYCARQVTSYGSD
YFDYWGKGTLVTVSSGGGGSGGGGSGGGGSAQAVVIQEPSFSVSPGGTVTLTCGLSSGSVSS
NYYPSWYQQTPGQTPRTLIYNTNTRSSGVPDRFSGSILGNKAALTITGAQADDESDYYCVLY
MGSGIRVFGGGTKVTVLGDVREPKSSDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSL
SLSPGK. (SEQ ID NO: 568)
>Her119_CDS
atggaagcaccagcgcagcttctcttcctcctgctactctggctcccagataccaccggtgaggtccagctggtgcagtctggggcagaggtgaaa
aagtccggggagtctctgaagatctcctgtaagggttctggatacagctttaccagtaattggatcggctgggtgcgccagatgcccgggaaaggc
ctggagtggatggggatcatctatcctggtgactctgataccagatacagcccgtccttccaaggccaggtcaccatctcagccgacaagtccgtca
gcaccgcctacctgcagtggagcagcctgaaggcctcggacaccgccatgtattattgtgcgagaatgctgacggactgtagtagtaccagctgct
attcagccggtatggacgtctggggcaaaggcaccctggtcaccgtctcgagtggaggcggcggttcaggcggaggtggctctggcggtggcg
gaagtgcacaggctgtggtgatccaggagccatcgttctcagtgtcccctggagggacagtcacactcacttgtggcttgagttctggctcagtctct
cctagttactaccccagctggtaccagcagaccccaggccaggctccacgcacactcatctacagcacaaacactcgctcttctggggtccctgat
cgcttctctggctccatccttgggaacaaagctgccctcaccatcacgggggcccaggcagatgatgaatctgattattactgtgtgctgtatatgggt
agtggctcttgggtgttcggcggagggaccaagctgaccgtcctaggtgacgtacgcgagcccaaatcttctgacaaaactcacacatgcccacc
gtgcccagcacctgaactcctgggtggaccgtcagtcttcctcttccccccaaaacccaaggacaccctcatgatctcccggacccctgaggtcac
atgcgtggtggtggacgtgagccacgaagaccctgaggtcaagttcaactggtacgtggacggcgtggaggtgcataatgccaagacaaagccg
cgggaggagcagtacaacagcacgtaccgtgtggtcagcgtcctcaccgtcctgcaccaggactggctgaatggcaaggagtacaagtgcaag
gtctccaacaaagccctcccagcccccatcgagaaaaccatctccaaagccaaagggcagccccgagaaccacaggtgtacaccctgcccccat
cccgggatgagctgaccaagaaccaggtcagcctgacctgcctggtcaaaggcttctatccaagcgacatcgccgtggagtgggagagcaatgg
gcagccggagaacaactacaagaccacgcctcccgtgctggactccgacggctccttcttcctctacagcaagctcaccgtggacaagagcaggt
ggcagcaggggaacgtcttctcatgctccgtgatgcatgaggctctgcacaaccactacacgcagaagagcctctccctgtctccgggtaaatga
(SEQ ID NO: 569)
>Her119_Protein_leader-stop
MEAPAQLLFLLLLWLPDTTGEVQLVQSGAEVKKSGESLKISCKGSGYSFTSNWIGWVRQMP
GKGLEWMGIIYPGDSDTRYSPSFQGQVTISADKSVSTAYLQWSSLKASDTAMYYCARMLTD
CSSTSCYSAGMDVWGKGTLVTVSSGGGGSGGGGSGGGGSAQAVVIQEPSFSVSPGGTVTLT
CGLSSGSVSPSYYPSWYQQTPGQAPRTLIYSTNTRSSGVPDRFSGSILGNKAALTITGAQADDE
SDYYCVLYMGSGSWVFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK. (SEQ ID NO: 570)
>Her120_CDS
atggaagcaccagcgcagcttctcttcctcctgctactctggctcccagataccaccggtgaggtgcagctggtgcagtctggggcagaggtgaaa
aagcccggggagtctctgaagatctcctgtaagggctctggatacagctttaccagctattggatcggctgggtgcgccagatgcccgggaaagg
cctggagtggatggggatcatctatcctggtgactctgataccagatacagcccgtccttccaaggccaggtcaccatctcagccgacaagtccatc
agcaccgcctacctgcagtggagcagcctgaaggcctcggacaccgccatgttttactgtgcgagactcaatgatagtagtggttatacgactaact
ttgactactggggccaaggcaccctggtcaccgtctcgagtggaggcggcggttcaggcggaggtggctctggcggtggcggaagtgcacagg
ctgtggtgatccaggagccatcgttctcagtgtcccctggagggacagtcacactcacttgtggcttgagctctggctcagtctctactcgttacaacc
ccagctggtaccagcagaccccaggccaggctccacgcacgctcatctacagtacaaacactcgttcttctggggtccctgaccgcttctctggctc
catccttgggaacaaagctgccctcaccatcacgggggcccaggcagatgatgaatctgattattactgtgcgctgtatatgggtagtggcatttggg
tgttcggcggagggaccaagctgaccgtcctaggtgacgtacgcgagcccaaatcttctgacaaaactcacacatgcccaccgtgcccagcacct
gaactcctgggtggaccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcgggaggagca
gtacaacagcacgtaccgtgtggtcagcgtcctcaccgtcctgcaccaggactggctgaatggcaaggagtacaagtgcaaggtctccaacaaag
ccctcccagcccccatcgagaaaaccatctccaaagccaaagggcagccccgagaaccacaggtgtacaccctgcccccatcccgggatgagc
tgaccaagaaccaggtcagcctgacctgcctggtcaaaggcttctatccaagcgacatcgccgtggagtgggagagcaatgggcagccggaga
acaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 571)
>Her120_Protein_leader-stop
MEAPAQLLFLLLLWLPDTTGEVQLVQSGAEVKKPGESLKISCKGSGYSFTSYWIGWVRQMP
GKGLEWMGIIYPGDSDTRYSPSFQGQVTISADKSISTAYLQWSSLKASDTAMFYCARLNDSS
GYTTNFDYWGQGTLVTVSSGGGGSGGGGSGGGGSAQAVVIQEPSFSVSPGGTVTLTCGLSSG
SVSTRYNPSWYQQTPGQAPRTLIYSTNTRSSGVPDRFSGSILGNKAALTITGAQADDESDYYC
ALYMGSGIWVFGGGTKLTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 572)
>Her121_CDS
atggaagcaccagcgcagcttctcttcctcctgctactctggctcccagataccaccggtcaggtgcagctggtgcaatctggggcagaggtgaaa
aagcccggggagtctctgaagatctcctgtaagggttctggatacagctttaccagccagtggatcgcctgggtgcgccagatgcccgggaaagg
cctggagtggatggggatcatctatcctggtgactctgatacgagatacagcccgtccttccaaggccaggtcaccatctcagccgacaagtccatc
aacaccgcctacctgcagtggagcagcctgaaggcctcggacaccgccatgtattactgtgcgagacattcggggagctctggagattactacca
ctactacggtatggacgtctggggccaagggacaatggtcaccgtctcgagtggaggcggcggttcaggcggaggtggctctggcggtggcgg
aagtgcacagactgtggtgatccaggagccatcgttctcagtgtcccctggagggacagtcacactcacttgtggcttgagctctggctcagtctcta
ctagttactaccccagctggtaccagcagaccccaggccaggctccacgcacgctcatctacagcacaaacactcgctcttctggggtccctgatc
gcttctctggctccatccttgggaacaaagctgccctcaccatcacgggggcccaggcagatgatggatctgattattactgtgtgctgtatatgggg
agtggcatttcggtgttcggcggagggaccaagctgaccgtcctaggtgacgtacgcgagcccaaatcttctgacaaaactcacacatgcccacc
gtgcccagcacctgaactcctgggtggaccgtcagtcttcctcttccccccaaaacccaaggacaccctcatgatctcccggacccctgaggtcac
atgcgtggtggtggacgtgagccacgaagaccctgaggtcaagttcaactggtacgtggacggcgtggaggtgcataatgccaagacaaagccg
cgggaggagcagtacaacagcacgtaccgtgtggtcagcgtcctcaccgtcctgcaccaggactggctgaatggcaaggagtacaagtgcaag
gtctccaacaaagccctcccagcccccatcgagaaaaccatctccaaagccaaagggcagccccgagaaccacaggtgtacaccctgcccccat
cccgggatgagctgaccaagaaccaggtcagcctgacctgcctggtcaaaggcttctatccaagcgacatcgccgtggagtgggagagcaatgg
gcagccggagaacaactacaagaccacgcctcccgtgctggactccgacggctccttcttcctctacagcaagctcaccgtggacaagagcaggt
ggcagcaggggaacgtcttctcatgctccgtgatgcatgaggctctgcacaaccactacacgcagaagagcctctccctgtctcc
gggtaaatga (SEQ ID NO: 573)
>Her121_Protein_leader-stop
MEAPAQLLFLLLLWLPDTTGQVQLVQSGAEVKKPGESLKISCKGSGYSFTSQWIAWVRQMP
GKGLEWMGIIYPGDSDTRYSPSFQGQVTISADKSINTAYLQWSSLKASDTAMYYCARHSGSS
GDYYHYYGMDVWGQGTMVTVSSGGGGSGGGGSGGGGSAQTVVIQEPSFSVSPGGTVTLTC
GLSSGSVSTSYYPSWYQQTPGQAPRTLIYSTNTRSSGVPDRFSGSILGNKAALTITGAQADDG
SDYYCVLYMGSGISVFGGGTKLTVLGDVREPKSSDKTHTCPPCPAPELLGGPSVFLFPPKPKD
TLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALH
NHYTQKSLSLSPGK. (SEQ ID NO: 574)
>Her122_CDS
atggaagcaccagcgcagcttctcttcctcctgctactctggctcccagataccaccggtcaggtgcagctgcaggagtcgggcccaggactggtg
aagccttcggagaccctgtccctcacctgcactgtctctggttactccattagcagtggttactactggggctggatccggcagcccccagggaggg
ggctggagtggattgggactatctatcatagtgggagcacctactacaacccgtccctcaagagtcgactcaccatatcagtagacacgtccgaga
accaattctccctgaagctgagttctgtgaccgccgcagacacggccgtgtattactgtgcgagagggatagcaggtcggacccattatgactactg
gggccaggggacaatggtcaccgtctcgagtggaggcggcggttcaggcggaggtggctctggcggtggcggaagtgcacaggctgtgctga
ctcagccgtcctcagtgtctggggccccagggcagagggtaaccatctcctgcagtgggagcagctccaacatcggggcaggttatgatgtacac
tggtaccagcagctcccaggagcagcccccaaactcctcatctatagtaacaatcatcggccctcaggggtccctgaccgattctctggctccaagt
ctggcacctcagcctccctggccatcactgggctccagactgaggatgaggctgattattactgccagtcctatgacagaagcctgagcggtaggg
tgttcggcggagggaccaagctgaccgtcctaggtgacgtacgcgagcccaaatcttctgacaaaactcacacatgcccaccgtgcccagcacct
gaactcctgggtggaccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcgggaggagca
gtacaacagcacgtaccgtgtggtcagcgtcctcaccgtcctgcaccaggactggctgaatggcaaggagtacaagtgcaaggtctccaacaaag
ccctcccagcccccatcgagaaaaccatctccaaagccaaagggcagccccgagaaccacaggtgtacaccctgcccccatcccgggatgagc
tgaccaagaaccaggtcagcctgacctgcctggtcaaaggcttctatccaagcgacatcgccgtggagtgggagagcaatgggcagccggaga
acaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 575)
>Her122_Protein_leader-stop
MEAPAQLLFLLLLWLPDTTGQVQLQESGPGLVKPSETLSLTCTVSGYSISSGYYWGWIRQPPG
RGLEWIGTIYHSGSTYYNPSLKSRLTISVDTSENQFSLKLSSVTAADTAVYYCARGIAGRTHY
DYWGQGTMVTVSSGGGGSGGGGSGGGGSAQAVLTQPSSVSGAPGQRVTISCSGSSSNIGAG
YDVHWYQQLPGAAPKLLIYSNNHRPSGVPDRFSGSKSGTSASLAITGLQTEDEADYYCQSYD
RSLSGRVFGGGTKLTVLGDVREPKSSDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSL
SLSPGK. (SEQ ID NO: 576)
>Her123_CDS
atggaagcaccagcgcagcttctcttcctcctgctactctggctcccagataccaccggtcaggtccagctggtgcagtctggggctgaggtgaag
aagcctggggcctcagtgaaggtctcctgcaaggcttctggttacacctttaccagctatggtatcagctgggtgcgacaggcccctggacaaggg
cttgagtggatgggatggatcagcgcttacaatggtaacacaaactatgcacagaagctccagggcagagtcaccatgaccacagacacatccac
gagcacagcctacatggagctgaggagcctgagatctgacgacacggccgtgtattactgtgcgagagtggggtcgggatattgtagtggtggta
gctgctacgtgggctggttcgacccctggggccgggggacaatggtcaccgtctcgagtggaggcggcggttcaggcggaggtggctctggcg
gtggcggaagtgcactttcttctgagctgactcaggaccctgctgtgtctgtggccttgggacagacagtcaagatcacatgccaaggagacagcct
cagtgcctattatgcaacctggtaccagcagaagccaggccaggcccctgtacttgtcatctatggtaaaaacaagcggccgtccgggatcccaga
ccgattctctggctccaagtcaggaaacacagcttccttgaccatcacgggggctcaggcggaagatgaggctgactattactgtaactcccggga
cagcagtggtaatgatcattatgtcttcggaactgggaccaagctgaccgttctaggtgacgtacgcgagcccaaatcttctgacaaaactcacacat
gcccaccgtgcccagcacctgaactcctgggtggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcataatgccaagac
aaagccgcgggaggagcagtacaacagcacgtaccgtgtggtcagcgtcctcaccgtcctgcaccaggactggctgaatggcaaggagtacaa
gtgcaaggtctccaacaaagccctcccagcccccatcgagaaaaccatctccaaagccaaagggcagccccgagaaccacaggtgtacaccct
gcccccatcccgggatgagctgaccaagaaccaggtcagcctgacctgcctggtcaaaggcttctatccaagcgacatcgccgtggagtgggag
agcaatgggcagccggagaacaactacaagaccacgcctcccgtgctggactccgacggctccttcttcctctacagcaagctcaccgtggacaa
gagcaggtggcagcaggggaacgtcttctcatgctccgtgatgcatgaggctctgcacaaccactacacgcagaagagcctctccctgtctccgg
gtaaatga (SEQ ID NO: 577)
>Her123_Protein_leader-stop
MEAPAQLLFLLLLWLPDTTGQVQLVQSGAEVKKPGASVKVSCKASGYTFTSYGISWVRQAP
GQGLEWMGWISAYNGNTNYAQKLQGRVTMTTDTSTSTAYMELRSLRSDDTAVYYCARVG
SGYCSGGSCYVGWFDPWGRGTMVTVSSGGGGSGGGGSGGGGSALSSELTQDPAVSVALGQ
TVKITCQGDSLSAYYATWYQQKPGQAPVLVIYGKNKRPSGIPDRFSGSKSGNTASLTITGAQA
EDEADYYCNSRDSSGNDHYVFGTGTKLTVLGDVREPKSSDKTHTCPPCPAPELLGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVM
HEALHNHYTQKSLSLSPGK. (SEQ ID NO: 578)
>Her124_CDS
atggaagcaccagcgcagcttctcttcctcctgctactctggctcccagataccaccggtgaggtgcagctggtgcagtctggggcagaagtcaag
aggcccggagagtctctgaagatctcctgtagggcctctggatacatctttacgaacaattgggtcgcctgggtgcgccagcagcccgggaaagg
cctggagtggatggggatcatctatcctggtgactctgacaccagatacagcccgtccttccaaggccaggtcactttctcggccgacacgtccatc
aacaccgcctacctacagtggaatagcctgaaggcctcggacaccgccacttacttctgtgcgcgagaggcctacaactcatacgaatattacggt
atggacgtctgggggcgagggaccacggtcaccgtctcgagtggaggcggcggttcaggcggaggtggctctggcggtggcggaagtgcaca
gactgtggtgatccaggagccatcgttctcagtgtcccctggagggacagtcacactcacttgtggcttgagctctggctcagtctctactaattacta
ccccagctggtaccagcagaccccaggccaggctccacgcacgctcatctacaacacaaacactcgctcttctggggtccctgatcgcttctctgg
ctccatccttgggaacaaagctgccctcaccatcacgggggcccaggcagatgatgaatctgattattactgtgtgctgtatatgggtagtggcatttc
ggtgttcggcggagggaccaaggtcaccgtcctaggtgacgtacgcgagcccaaatcttctgacaaaactcacacatgcccaccgtgcccagca
cctgaactcctgggtggaccgtcagtcttcctcttccccccaaaacccaaggacaccctcatgatctcccggacccctgaggtcacatgcgtggtgg
tggacgtgagccacgaagaccctgaggtcaagttcaactggtacgtggacggcgtggaggtgcataatgccaagacaaagccgcgggaggagc
agtacaacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccgggatgag
ctgaccaagaaccaggtcagcctgacctgcctggtcaaaggcttctatccaagcgacatcgccgtggagtgggagagcaatgggcagccggag
aacaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 579)
>Her124_Protein_leader-stop
MEAPAQLLFLLLLWLPDTTGEVQLVQSGAEVKRPGESLKISCRASGYIFTNNWVAWVRQQP
GKGLEWMGIIYPGDSDTRYSPSFQGQVTFSADTSINTAYLQWNSLKASDTATYFCAREAYNS
YEYYGMDVWGRGTTVTVSSGGGGSGGGGSGGGGSAQTVVIQEPSFSVSPGGTVTLTCGLSS
GSVSTNYYPSWYQQTPGQAPRTLIYNTNTRSSGVPDRFSGSILGNKAALTITGAQADDESDYY
CVLYMGSGISVFGGGTKVTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYT
QKSLSLSPGK. (SEQ ID NO: 580)
>Her125_CDS
atggaagcaccagcgcagcttctcttcctcctgctactctggctcccagataccaccggtcaggtccagctggtgcagtctggaggagaggtgaaa
aagcccggggagtctctgaagatctcctgtaaggtttctggagacaagtttgccaactactggatcgcctgggtgcgccaggtgcccgggagagg
cctggagtggatgggaatcatctatcctagtgactctgatgtcagatatagtccgtccttccaaggccaagtcaccatgtcagccgacaagtccacca
gcaccgcctacttgcagttgagcagcctgaaggcctcggacaccgccatgtattactgtgcaagacaggtgggtggactggttactacagacactg
actcctacttctacggcatggacgtctggggccaaggaaccctggtcaccgtctcgagtggaggcggcggttcaggcggaggtggctctggcggt
ggcggaagtgcacaggctgtggtgatccaggagccatcgttctcagtgtcccctggagggacagtcacactcacttgtggcttgagctctggccca
gtctctactagttactaccccagctggttccaacagaccccaggccaggctccacgcacgctcatctacagcacaaacactcgctcttctggggtcc
ctgatcgcttctctggctccatccttgggaacaaagctgccctcaccatcacgggggcccaggcagatgatgaatctgattattactgtgtgttgtatgt
gggtagtggcatttcgctattcggcggggggaccaaggtcaccgtcctaggtgacgtacgcgagcccaaatcttctgacaaaactcacacatgccc
accgtgcccagcacctgaactcctgggtggaccgtcagtcttcctcttccccccaaaacccaaggacaccctcatgatctcccggacccctgaggt
cacatgcgtggtggtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaaggagtacaagtgc
aaggtctccaacaaagccctcccagcccccatcgagaaaaccatctccaaagccaaagggcagccccgagaaccacaggtgtacaccctgccc
ccatcccgggatgagctgaccaagaaccaggtcagcctgacctgcctggtcaaaggcttctatccaagcgacatcgccgtggagtgggagagca
atgggcagccggagaacaactacaagaccacgcctcccgtgctggactccgacggctccttcttcctctacagcaagctcaccgtggacaagagc
aggtggcagcaggggaacgtcttctcatgctccgtgatgcatgaggctctgcacaaccactacacgcagaagagcctctccctgtctccgggtaaa
tga (SEQ ID NO: 581)
>Her125_Protein_leader-stop
MEAPAQLLFLLLLWLPDTTGQVQLVQSGGEVKKPGESLKISCKVSGDKFANYWIAWVRQVP
GRGLEWMGIIYPSDSDVRYSPSFQGQVTMSADKSTSTAYLQLSSLKASDTAMYYCARQVGG
LVTTDTDSYFYGMDVWGQGTLVTVSSGGGGSGGGGSGGGGSAQAVVIQEPSFSVSPGGTVT
LTCGLSSGPVSTSYYPSWFQQTPGQAPRTLIYSTNTRSSGVPDRFSGSILGNIKAALTITGAQAD
DESDYYCVLYVGSGISLFGGGTKVTVLGDVREPKSSDKTHTCPPCPAPELLGGPSVFLFPPKP
KDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK. (SEQ ID NO: 582)
>Her126_CDS
atggaagcaccagcgcagcttctcttcctcctgctactctggctcccagataccaccggtgaggtgcagctggtggagtccgggggaggcttggtc
cagccgggggggtccctaaaactttcctgtgcagcctctgggctcaatttcgatatctctactgtgcactgggtccgccaggcttccgggaaagggc
tggagtggattggccgtattagaagcaaagcttacaattatgcgacagcatatactgagtcgctgaagggcaggttcatcatctccagagatgagtca
aagaatacggcggatctgcaaatcaacagcctgaaaaccgaggacacggccacatattactgtagtatgaccttcggtgactactactactacggc
atggacgtctggggccggggcaccctggtcaccgtctcgagtggaggcggcggttcaggcggaggtggctctggcggtggcggaagtgcaca
ggctgtgctgactcagccgtcctcagtgtctggggccccagggcagagggtcaccatcacctgcactggaagcagctccaacatcggggccggt
tacgatgttcactggtaccagcaacttccaggaacagccccaaactcctcatctatagtaattcttatcggccctctggggtctctgaccgattctctg
gctccaagtctggcacctcagcctccctggtcatcgctggactccaggctgaggatgaggctgattattactgtcagtcctatgacagcagtcattgg
tttttcggcggagggaccaagctgaccgtcctaggtgacgtacgcgagcccaaatcttctgacaaaactcacacatgcccaccgtgcccagcacct
gaactcctgggtggaccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcgggaggagca
gtacaacagcacgtaccgtgtggtcagcgtcctcaccgtcctgcaccaggactggctgaatggcaaggagtacaagtgcaaggtctccaacaaag
ccctcccagcccccatcgagaaaaccatctccaaagccaaagggcagccccgagaaccacaggtgtacaccctgcccccatcccgggatgagc
tgaccaagaaccaggtcagcctgacctgcctggtcaaaggcttctatccaagcgacatcgccgtggagtgggagagcaatgggcagccggaga
acaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 583)
>Her126_Protein_leader-stop
MEAPAQLLFLLLLWLPDTTGEVQLVESGGGLVQPGGSLKLSCAASGLNFDISTVHWVRQAS
GKGLEWIGRIRSKAYNYATAYTESLKGRFIISRDESKNTADLQINSLKTEDTATYYCSMTFGD
YYYYGMDVWGRGTLVTVSSGGGGSGGGGSGGGGSAQAVLTQPSSVSGAPGQRVTITCTGSS
SNIGAGYDVHWYQQLPGTAPKLLIYSNSYRPSGVSDRFSGSKSGTSASLVIAGLQAEDEADY
YCQSYDSSHWFFGGGTKLTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYT
QKSLSLSPGK. (SEQ ID NO: 584)
>Her127_CDS
atggaagcaccagcgcagcttctcttcctcctgctactctggctcccagataccaccggtcaggtccagctggtgcagtctggaggagaggtgaaa
aagcccggggagtctctgaagatctcctgtaaggtttctggagacagctttaccaactactggatcgcctgggtgcgccagatgcccgggagaggc
ctggagtggatgggaatcatctatcctagtgactctgatgtcagatatagtccgtccttccaaggccaggtcaccatgtcagccgacaagtccatcag
caccgcctacctgcagttgagcagcctgaaggcctcggacaccgccatgtattactgtgcaagacaggtgggtggactggttactacagacactga
ctcctacttctacggcatggacgtctggggcagaggcaccctggtcaccgtctcgagtggaggcggcggttcaggcggaggtggctctggcggt
ggcggaagtgcacagactgtggtgatccaggagccatcgttctcagtgtcccctggagggacagtcacactcacttgtgccttgaactccggctca
gtctctactaattactaccccagctggtaccagcagaccccaggccaggctccacgcacgctcatccacagcacaaacactcgctcttctggggtc
cctgatcgcttctctggctccatccttgggaacaatgctgccctcaccatcacgggggcccaggcagaggatgaatctgattattactgtgcgctatat
atgggtagtggcatttcgatattcggcggagggaccaagctgaccgtcctaggtgacgtacgcgagcccaaatcttctgacaaaactcacacatgc
ccaccgtgcccagcacctgaactcctgggtggaccgtcagtcttcctcttccccccaaaacccaaggacaccctcatgatctcccggacccctgag
gtcacatgcgtggtggtggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatggcaaggagtacaagt
gcaaggtctccaacaaagccctcccagcccccatcgagaaaaccatctccaaagccaaagggcagccccgagaaccacaggtgtacaccctgc
ccccatcccgggatgagctgaccaagaaccaggtcagcctgacctgcctggtcaaaggcttctatccaagcgacatcgccgtggagtgggagag
caatgggcagccggagaacaactacaagaccacgcctcccgtgctggactccgacggctccttcttcctctacagcaagctcaccgtggacaaga
gcaggtggcagcaggggaacgtcttctcatgctccgtgatgcatgaggctctgcacaaccactacacgcagaagagcctctccctgtctccgggta
aatga (SEQ ID NO: 585)
>Her127_Protein_leader-stop
MEAPAQLLFLLLLWLPDTTGQVQLVQSGGEVKKPGESLKISCKVSGDSFTNYWIAWVRQMP
GRGLEWMGIIYPSDSDVRYSPSFQGQVTMSADKSISTAYLQLSSLKASDTAMYYCARQVGGL
VTTDTDSYFYGMDVWGRGTLVTVSSGGGGSGGGGSGGGGSAQTVVIQEPSFSVSPGGTVTL
TCALNSGSVSTNYYPSWYQQTPGQAPRTLIHSTNTRSSGVPDRFSGSILGNNAALTITGAQAE
DESDYYCALYMGSGISIFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK. (SEQ ID NO: 586)
>Her128_CDS
atggaagcaccagcgcagcttctcttcctcctgctactctggctcccagataccaccggtgaggtccagctggtgcagtctggagcagaggtgaaa
aagcccggggagtctctgaagatctcctgtaagggttctggatacagctttcccagctactggatcggctgggtgcgccagatgcccgggaaagg
cctggagtggatggggatcatctatcctggtgactctgaaaccagatacagcccgtccttccaaggccaggtcaccatctcagccgacaagtccatc
agcaccgcctacctgcagtggagcagcctgaaggcctcggacaccgccatgtattactgtgtgagacatctaaaaccagtggctggtcccgcttgg
cacgactacggtatggacgtctggggccagggcaccctggtcaccgtctcgagtggaggcggcggttcaggcggaggtggctctggcggtggc
ggaagtgcacaggctgtggtgctccaggagccatcgatctcagtgtcccctggagggacagtcacactcacttgtggcttaacctctgactcagtct
cgactacttactaccccagctggtaccagcagaccccaggccagactccacgcacactcagctacagcacaaatactcgctcttctggggtccctg
atcgcttctctggctccatccttgggaacaaagctgccctcaccatcgcgggggcccaggcagatgatgaagctgattattactgtgccctatatatg
ggcagtggcatttgggtgttcggcggagggacccagctcaccgttttaagtgacgtacgcgagcccaaatcttctgacaaaactcacacatgccca
ccgtgcccagcacctgaactcctgggtggaccgtcagtcttcctcttccccccaaaacccaaggacaccctcatgatctcccggacccctgaggtc
acatgcgtggtggtggacgtgagccacgaagaccctgaggtcaagttcaactggtacgtggacggcgtggaggtgcataatgccaagacaaagc
cgcgggaggagcagtacaacagcacgtaccgtgtggtcagcgtcctcaccgtcctgcaccaggactggctgaatggcaaggagtacaagtgca
aggtctccaacaaagccctcccagcccccatcgagaaaaccatctccaaagccaaagggcagccccgagaaccacaggtgtacaccctgcccc
catcccgggatgagctgaccaagaaccaggtcagcctgacctgcctggtcaaaggcttctatccaagcgacatcgccgtggagtgggagagcaa
tgggcagccggagaacaactacaagaccacgcctcccgtgctggactccgacggctccttcttcctctacagcaagctcaccgtggacaagagca
ggtggcagcaggggaacgtcttctcatgctccgtgatgcatgaggctctgcacaaccactacacgcagaagagcctctccctgtctccgggtaaat
ga (SEQ ID NO: 587)
>Her128_Protein_leader-stop
MEAPAQLLFLLLLWLPDTTGEVQLVQSGAEVKKPGESLKISCKGSGYSFPSYWIGWVRQMP
GKGLEWMGIIYPGDSETRYSPSFQGQVTISADKSISTAYLQWSSLKASDTAMYYCVRHLKPV
AGPAWHDYGMDVWGQGTLVTVSSGGGGSGGGGSGGGGSAQAVVLQEPSISVSPGGTVTLT
CGLTSDSVSTTYYPSWYQQTPGQTPRTLSYSTNTRSSGVPDRFSGSILGNKAALTIAGAQADD
EADYYCALYMGSGIWVFGGGTQLTVLSDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK. (SEQ ID NO: 588)
>Her129_CDS
atggaagcaccagcgcagcttctcttcctcctgctactctggctcccagataccaccggtgaggtccagctggtacagtctggggctgaggtgagg
aagcctggggcctcagtcaaggtctcctgcagggcttctggatacaacttcaaagactactatttgcactgggtgcgccaggcccctggagaaggg
cttgagtggatggggtggatcaaccctcacgctggtaccacaaaatatgcacagaattttcagcacaggattattatgaccagggacacgaccatca
ccacagcctacatggaactgagcagtctgaaatctgacgacacagccatttatttctgtaccagatactactttgacagtagtggttattttaggttcgac
ccctggggccaagggacaatggtcaccgtctcgagtggaggcggcggttcaggcggaggtggctctggcggtggcggaagtgcacagtctgtc
gtgacgcagccgccctcagtgtctggggccccaggacagaaggtcaccatctcctgctctggaagcagctccaacattgggaataattatgtatcct
ggtaccagcagctcccaggaacagcccccaaactcctcatttatgacaataataagcgaccctcagggattcctgaccgattctctggctccaagtct
ggcacgtcagccaccctgggcatcaccggactccagactggggacgaggccgattattactgcggaacatgggatagcagcctgagtgctggg
gtgttcggcggagggaccaaggtcaccgtcctaggtgacgtacgcgagcccaaatcttctgacaaaactcacacatgcccaccgtgcccagcacc
tgaactcctgggtggaccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcgggaggagca
gtacaacagcacgtaccgtgtggtcagcgtcctcaccgtcctgcaccaggactggctgaatggcaaggagtacaagtgcaaggtctccaacaaag
ccctcccagcccccatcgagaaaaccatctccaaagccaaagggcagccccgagaaccacaggtgtacaccctgcccccatcccgggatgagc
tgaccaagaaccaggtcagcctgacctgcctggtcaaaggcttctatccaagcgacatcgccgtggagtgggagagcaatgggcagccggaga
acaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 589)
>Her129_Protein_leader-stop
MEAPAQLLFLLLLWLPDTTGEVQLVQSGAEVRKPGASVKVSCRASGYNFKDYYLHWVRQA
PGEGLEWMGWINPHAGTTKYAQNFQHRIIMTRDTTITTAYMELSSLKSDDTAIYFCTRYYFD
SSGYFRFDPWGQGTMVTVSSGGGGSGGGGSGGGGSAQSVVTQPPSVSGAPGQKVTISCSGSS
SNIGNNYVSWYQQLPGTAPKLLIYDNNKRPSGIPDRFSGSKSGTSATLGITGLQTGDEADYYC
GTWDSSLSAGVFGGGTKVTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYT
QKSLSLSPGK. (SEQ ID NO: 590)
>Her130_CDS
atggaagcaccagcgcagcttctcttcctcctgctactctggctcccagataccaccggtgaggtgcagctggtgcagtctggggcagagatgaaa
aagcccggggagtctctgaagatatcctgcaagacttctggatacagctttaccggctcctggatcgcctgggtgcgccagatgcccgggaaagg
cctggagtggatggggatcatctatcctggtgactctgacaccagatacagcccgtccttccaaggccaggtcaccatctcagccgacaagtccatc
agcaccgcctacctgcagtggagcagcctgaaggcctcggacaccgccatgtattactgtgcgaggatttatagtgactcgggttacaattggttcg
actcttggggcaggggaaccctggtcaccgtctcgagtggaggcggcggttcaggcggaggtggctctggcggtggcggaagtgcacagactg
tggtgatccaggagccatcgttctcagtgtcccctggagggacagtcacactcacttgtggcttgagctctggctcagtctctaatagtcactacccca
gctggtatcagcagaccccaggccaggctccacgaacgctcatctacagcacaaacactcgctcttctggggtccctgatcgcttctctggctccat
ccttgggaacaaagctgccctcaccatcacgggggcccaggcggatgatgaatctgatttttactgtctgctatatctgggtagtggcatttcggtatt
cggcggagggaccaagctgaccgtcctaggtgacgtacgcgagcccaaatcttctgacaaaactcacacatgcccaccgtgcccagcacctgaa
ctcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 591)
>Her130_Protein_leader-stop
MEAPAQLLFLLLLWLPDTTGEVQLVQSGAEMKKPGESLKISCKTSGYSFTGSWIAWVRQMP
GKGLEWMGIIYPGDSDTRYSPSFQGQVTISADKSISTAYLQWSSLKASDTAMYYCARIYSDSG
YNWFDSWGRGTLVTVSSGGGGSGGGGSGGGGSAQTVVIQEPSFSVSPGGTVTLTCGLSSGSV
SNSHYPSWYQQTPGQAPRTLIYSTNTRSSGVPDRFSGSILGNKAALTITGAQADDESDFYCLL
YLGSGISVFGGGTKLTVLGDVREPKSSDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 592)
>Her131_CDS
atggaagcaccagcgcagcttctcttcctcctgctactctggctcccagataccaccggtgaagtgcagctggtgcagtctggagcagaggtgaaa
aagcccggggagtctctgaagatctcctgtaagggttctggatacaactttaacactcactggatcgggtgggtgcgccagatgcccgggaaaggc
ctggagtggatggggctcatctaccctgatgactctgacacccgattcagcccgtccttcgaaggccaggtcaccctctcagccgacaggtccatc
agtaccgcctacctgcagtggaccagcctgaaggcctcggacaccgccatgtattactgtgcgagatacaaaaaaagtagtggttattacacagga
tatggtatggacgtctggggccgagggacaatggtcaccgtctcgagtggaggcggcggttcaggcggaggtggctctggcggtggcggaagt
gcacagactgtggtgatccaggagccatcgttctcagtgtcccctggagggacagtcacactcacttgtggcttgagctctggctcagtctctactag
ttactaccccacctggtaccagcagaccccaggccaggctccacgcacgctcatctatagcacaaacagtcgcttttctggggtccctgatcgcttct
ctggctccatccttgggagcaaagctgccctcaccatcacgggggcccaggcagatgatgaatctgattattactgtgtgctatatatgggtagtggc
atttcggtgttcggcggagggaccaagctgaccgtcctaggtgacgtacgcgagcccaaatcttctgacaaaactcacacatgcccaccgtgccca
gcacctgaactcctgggtggaccgtcagtcttcctcttccccccaaaacccaaggacaccctcatgatctcccggacccctgaggtcacatgcgtg
gtggtggacgtgagccacgaagaccctgaggtcaagttcaactggtacgtggacggcgtggaggtgcataatgccaagacaaagccgcgggag
gagcagtacaacagcacgtaccgtgtggtcagcgtcctcaccgtcctgcaccaggactggctgaatggcaaggagtacaagtgcaaggtctccaa
caaagccctcccagcccccatcgagaaaaccatctccaaagccaaagggcagccccgagaaccacaggtgtacaccctgcccccatcccggga
tgagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtctccg
ggtaaatga (SEQ ID NO: 593)
>Her131_Protein_leader-stop
MEAPAQLLFLLLLWLPDTTGEVQLVQSGAEVKKPGESLKISCKGSGYNFNTHWIGWVRQMP
GKGLEWMGLIYPDDSDTRFSPSFEGQVTLSADRSISTAYLQWTSLKASDTAMYYCARYKKSS
GYYTGYGMDVWGRGTMVTVSSGGGGSGGGGSGGGGSAQTVVIQEPSFSVSPGGTVTLTCG
LSSGSVSTSYYPTWYQQTPGQAPRTLIYSTNSRFSGVPDRFSGSILGSKAALTITGAQADDESD
YYCVLYMGSGISVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 594)
>Her132_CDS
atggaagcaccagcgcagcttctcttcctcctgctactctggctcccagataccaccggtcaggtgcagctgcaggagtccggcccaggactcgtg
aagcctgcggggactctgtccctcacctgcgctgtctccggtgactccatcagcagcaatcactggtggaattgggtccgccagtccccagggaag
ggactggaatggattggtgaaatctttcatagtgacattcgcatcctcaacccgtccctcaagaggcgcgtctccatgtcagtcgacaggtccaagg
accaattctccctgcaactgacctctgtgaccgccgcggacacggccgtgtattactgtgcgagaggtttccatggtgactccggcagaggacttga
cacctggggcagaggaaccctggtcaccgtctcgagtggaggcggcggttcaggcggaggtggctctggcggtggcggaagtgcactttcttct
gagctgactcaggaccctgctgtgtctgtggccttgggacagacagtcagggtcacatgccaaggagacggcctcagaagttattatgcaagctgg
taccagcagaagccagggcaggcccctgtccttgtcatgtatgggaacaacaaccggccctcagggatcccagaccgattctctggctccagctc
gggaaacacagcttccttgaccatcactggggctcaggcggaagatgaggctgtctattattgtaattcgcgggacagcggtgctaaccatctgga
ggttttcggcggagggaccaaggtcaccgtcctaggtgacgtacgcgagcccaaatcttctgacaaaactcacacatgcccaccgtgcccagcac
ctgaactcctgggtggaccgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaaagccgcgggaggagc
agtacaacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccgggatgag
ctgaccaagaaccaggtcagcctgacctgcctggtcaaaggcttctatccaagcgacatcgccgtggagtgggagagcaatgggcagccggag
aacaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 595)
>Her132_Protein_leader-stop
MEAPAQLLFLLLLWLPDTTGQVQLQESGPGLVKPAGTLSLTCAVSGDSISSNHWWNWVRQS
PGKGLEWIGEIFHSDIRILNPSLKRRVSMSVDRSKDQFSLQLTSVTAADTAVYYCARGFHGDS
GRGLDTWGRGTLVTVSSGGGGSGGGGSGGGGSALSSELTQDPAVSVALGQTVRVTCQGDG
LRSYYASWYQQKPGQAPVLVMYGNNNRPSGIPDRFSGSSSGNTASLTITGAQAEDEAVYYC
NSRDSGANHLEVFGGGTKVTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 596)
>Her133_CDS
atggaagcaccagcgcagcttctcttcctcctgctactctggctcccagataccaccggtgaggtgcagctggtggagtctgggggaggcttggca
cagcctggggggtccctgagactctcctgtgcagcctctggattaacctttaccacctatgccatgagctgggtccgccaggctccagggaagggg
ctggagtgggtctcaagtattagtggaagtggtcatagcacatattacgcagactccgtgaagggccgcttcaccatctccagagacatttccaaga
acacgttgtatctgcaaatgaacagcctcagagccgaggacacggccgtctattactgtgcgaaagattcgtcggcttttgggtttgtacacggtgctt
ttgatatctggggccagggaaccctggtcaccgtctcgagtggaggcggcggttcaggcggaggtggctctggcggtggcggaagtgcactttct
tctgagctgactcaggaccctgctgcgtctgtggccttgggacagacagtcagcatcacatgccaaggagacagcctcagaaactattgggctagc
tggtaccagcagaagccaggacaggcccctgtacttgtcatctatggtaaaaatacccggccctcagggatcccagaccgattctctggctccacct
caggaaacacagcttccttgaccatcactggggctcaggcggaggatgaggctgactattactgcaactcccgggacagtggtcaccgtcttctttt
cggcggagggaccaagctgaccgtcctaggtgacgtacgcgagcccaaatcttctgacaaaactcacacatgcccaccgtgcccagcacctgaa
ctcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 597)
>Her133_Protein_leader-stop
MEAPAQLLFLLLLWLPDTTGEVQLVESGGGLAQPGGSLRLSCAASGLTFTTYAMSWVRQAP
GKGLEWVSSISGSGHSTYYADSVKGRFTISRDISKNTLYLQMNSLRAEDTAVYYCAKDSSAF
GFVHGAFDIWGQGTLVTVSSGGGGSGGGGSGGGGSALSSELTQDPAASVALGQTVSITCQG
DSLRNYWASWYQQKPGQAPVLVIYGKNTRPSGIPDRFSGSTSGNTASLTITGAQAEDEADYY
CNSRDSGHRLLFGGGTKLTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 598)
>Her134_CDS
atggaagcaccagcgcagcttctcttcctcctgctactctggctcccagataccaccggtgaggtgcagctggtggagtccggagcagaagtcaaa
aagcccggagagtctctgaagatctcctgtaaggcttctggatacatctttacgaacaattggatcgcctgggtgcggcagcagcccgggaaaggc
ctggagtggatgggaatcatctatcctggtgactctgacaccagatacagcccgtccttccagggccgggtcactttctcagccgacacgtccatca
acaccgcctacctccagtggagtagcctgaaggcctcggacaccgccacttactactgtgcgagagaggcctacaactcatacgagtactacggt
atggacgtctggggccaagggacaatggtcaccgtctcgagtggaggcggcggttcaggcggaggtggctctggcggtggcggaagtgcaca
gactgtggtgctccaggagccagcgttctcagtgtcccctggagggacagtcacactcacctgtggcttgagctctggctcagtctctactagttact
accccagttggtaccagcagaccccaggccagcctccacgcacgctcatctacaacacaaacacccgctcttctggggtctctgatcgcttctctgg
ctccatccttgggaacaaagctgccctcaccatcacgggggcccaggccgaagatgaatctgattattactgtgttctgtatatgggtagtggcatttc
ggtattcggcggggggaccaagctgaccgtcctaggtgacgtacgcgagcccaaatcttctgacaaaactcacacatgcccaccgtgcccagca
cctgaactcctgggtggaccgtcagtcttcctcttccccccaaaacccaaggacaccctcatgatctcccggacccctgaggtcacatgcgtggtgg
tggacgtgagccacgaagaccctgaggtcaagttcaactggtacgtggacggcgtggaggtgcataatgccaagacaaagccgcgggaggagc
agtacaacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccgggatgag
ctgaccaagaaccaggtcagcctgacctgcctggtcaaaggcttctatccaagcgacatcgccgtggagtgggagagcaatgggcagccggag
aacaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 599)
>Her134_Protein_leader-stop
MEAPAQLLFLLLLWLPDTTGEVQLVESGAEVKKPGESLKISCKASGYIFTNNWIAWVRQQPG
KGLEWMGIIYPGDSDTRYSPSFQGRVTFSADTSINTAYLQWSSLKASDTATYYCAREAYNSY
EYYGMDVWGQGTMVTVSSGGGGSGGGGSGGGGSAQTVVLQEPAFSVSPGGTVTLTCGLSS
GSVSTSYYPSWYQQTPGQPPRTLIYNTNTRSSGVSDRFSGSILGNKAALTITGAQAEDESDYY
CVLYMGSGISVFGGGTKLTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 600)
>Her135_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgaaagatttctggtttggactaccaccttccttctttg
actcttggggccaagggacaatggtcaccgtctcgagtggaggcggcggttcaggcggaggtggctctggcggtggcggaagtgcacagtctgt
gctgactcagccaccctcagtgtccgtgtccccaggacagaaggccagcatcacctgctctggagaaagaatgggggataaatatgctgcctggt
atcagcagaagccaggccagtcacctatactggtcatctatcaagatacaaagcggccctcagggatccctgagcgattctctggctccaactctgg
gaacacagccacgttgaccatcagcgggacccaggacatggatgaggctgactattactgtcaggtgtgggacagcagcactggggtattcggc
ggagggaccaaggtcaccgtcctaggtgacgtacgcgagcccaaatcttctgacaaaactcacacatgcccaccgtgcccagcacctgaactcct
gggtggaccgtcagtcttcctcttccccccaaaacccaaggacaccctcatgatctcccggacccctgaggtcacatgcgtggtggtggacgtgag
ccacgaagaccctgaggtcaagttcaactggtacgtggacggcgtggaggtgcataatgccaagacaaagccgcgggaggagcagtacaacag
cacgtaccgtgtggtcagcgtcctcaccgtcctgcaccaggactggctgaatggcaaggagtacaagtgcaaggtctccaacaaagccctcccag
cccccatcgagaaaaccatctccaaagccaaagggcagccccgagaaccacaggtgtacaccctgcccccatcccgggatgagctgaccaaga
accaggtcagcctgacctgcctggtcaaaggcttctatccaagcgacatcgccgtggagtgggagagcaatgggcagccggagaacaactacaa
gaccacgcctcccgtgctggactccgacggctccttcttcctctacagcaagctcaccgtggacaagagcaggtggcagcaggggaacgtcttct
catgctccgtgatgcatgaggctctgcacaaccactacacgcagaagagcctctccctgtctccgggtaaatga (SEQ ID NO: 601)
>Her135_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAKDFWF
GLPPSFFDSWGQGTMVTVSSGGGGSGGGGSGGGGSAQSVLTQPPSVSVSPGQKASITCSGER
MGDKYAAWYQQKPGQSPILVIYQDTKRPSGIPERFSGSNSGNTATLTISGTQDMDEADYYCQ
VWDSSTGVFGGGTKVTVLGDVREPKSSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 602)
>Her136_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacatggccgtgtattactgtgcgaggactcccgggtatagcagtggctggtactcgg
tttggggccggggcaccctggtcaccgtctcgagtggaggcggcggttcaggcggaggtggctctggcggtggcggaagtgcacagtctgtcgt
gacgcagccgccctcagtgtctggggccccagggcagagggtcaccatctcctgtactgggagcagctccaacatcggggcagggtatgatgtt
cactggtaccagcaggttccaggaacagcccccaaactcctcatctatggtaacaacaatcggccctcgggggtccctgaccgattctctggctcc
aagtctggcacctcagcctccctggccatcactgggctccagcctgaggatgaagttgattattactgccagtcctatgaccgcagcctgagtggtta
tatcttcggaagtgggaccaaggtcaccgtcctaggtgacgtacgcgagcccaaatcttctgacaaaactcacacatgcccaccgtgcccagcacc
tgaactcctgggtggaccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcgggaggagca
gtacaacagcacgtaccgtgtggtcagcgtcctcaccgtcctgcaccaggactggctgaatggcaaggagtacaagtgcaaggtctccaacaaag
ccctcccagcccccatcgagaaaaccatctccaaagccaaagggcagccccgagaaccacaggtgtacaccctgcccccatcccgggatgagc
tgaccaagaaccaggtcagcctgacctgcctggtcaaaggcttctatccaagcgacatcgccgtggagtgggagagcaatgggcagccggaga
acaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 603)
>Her136_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDMAVYYCARTPGY
SSGWYSVWGRGTLVTVSSGGGGSGGGGSGGGGSAQSVVTQPPSVSGAPGQRVTISCTGSSSN
IGAGYDVHWYQQVPGTAPKLLIYGNNNRPSGVPDRFSGSKSGTSASLAITGLQPEDEVDYYC
QSYDRSLSGYIFGSGTKVTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 604)
>Her137_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgaaagatctgaacccttattcagtggtaactctcgg
tatggacgtctggggcagagggacaatggtcaccgtctcgagtggaggcggcggttcaggcggaggtggctctggcggtggcggaagtgcaca
gtctgtgctgactcagccaccctcggtgtcagtggccccaggacaggcggccaggattccctgtgggggagacaacattggaagtaagagtgttc
actggtaccagcagaggccaggccaggcccctgtcctggtcgtctttgatgatagtgaccggccctcagggatccctgagcgattctctggctcca
attctgggcacacggccaccctgaccatcaacagggtcgaacccggggatgaggccgagtattattgtgaggtgtgggatggtggcgagagaca
tgtggtattcggcggagggaccaagctgaccgtcctaggtgacgtacgcgagcccaaatcttctgacaaaactcacacatgcccaccgtgcccag
cacctgaactcctgggtggaccgtcagtcttcctcttccccccaaaacccaaggacaccctcatgatctcccggacccctgaggtcacatgcgtggt
ggtggacgtgagccacgaagaccctgaggtcaagttcaactggtacgtggacggcgtggaggtgcataatgccaagacaaagccgcgggagg
agcagtacaacagcacgtaccgtgtggtcagcgtcctcaccgtcctgcaccaggactggctgaatggcaaggagtacaagtgcaaggtctccaac
aaagccctcccagcccccatcgagaaaaccatctccaaagccaaagggcagccccgagaaccacaggtgtacaccctgcccccatcccgggat
gagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtctcc
gggtaaatga (SEQ ID NO: 605)
>Her137_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAKDLNP
YSVVTLGMDVWGRGTMVTVSSGGGGSGGGGSGGGGSAQSVLTQPPSVSVAPGQAARIPCG
GDNIGSKSVHWYQQRPGQAPVLVVFDDSDRPSGIPERFSGSNSGHTATLTINRVEPGDEAEYY
CEVWDGGERHVVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 606)
>Her138_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccataagctgggtccgccaggctccagggaaggggc
tggagtgggtctcagctattagtggtagtggtggtagcacatactacgcagactccgtgaagggccggttcaccatctccagagacaattccaagaa
cacgctgtatctgcaaatgaacagcctgagagccgaggacacggccgtgtattactgtgcgagagattcgagtagggtgggagcttatctggtgttt
gactactggggccgggggacaatggtcaccgtctcgagtggaggcggcggttcaggcggaggtggctctggcggtggcggaagtgcacagtct
gtgctgacgcagccgccctcagtgtctggggccccagggcagagggtcaccatctcctgcactgggagcagctccaacatcggggcaggttatg
atgtacactggtaccagcagcttccaggaacagcccccaaactcctcatctatggtaacagcaaacgcccctcaggggtccctgaccgattctctgg
ctccaagtctggcacctcagcctccctggccctcactgggctccaggctgaggatgaggctgattattactgccagtcctatgacagcagcctgagt
ggttatgtcttcggaactgggaccaaggtcaccgtcctaggtgacgtacgcgagcccaaatcttctgacaaaactcacacatgcccaccgtgccca
gcacctgaactcctgggtggaccgtcagtcttcctcttccccccaaaacccaaggacaccctcatgatctcccggacccctgaggtcacatgcgtg
gtggtggacgtgagccacgaagaccctgaggtcaagttcaactggtacgtggacggcgtggaggtgcataatgccaagacaaagccgcgggag
gagcagtacaacagcacgtaccgtgtggtcagcgtcctcaccgtcctgcaccaggactggctgaatggcaaggagtacaagtgcaaggtctccaa
caaagccctcccagcccccatcgagaaaaccatctccaaagccaaagggcagccccgagaaccacaggtgtacaccctgcccccatcccggga
tgagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtctcc
gggtaaatga (SEQ ID NO: 607)
>Her138_Protein_leader-stop
MEAPAQLLFLLLLWLPDTTGEVQLLESGGGLVQPGGSLRLSCAASGFTFSSYAISWVRQAPG
KGLEWVSAISGSGGSTYYADSVKGRFTISRDNSKNTLYLQMNSLRAEDTAVYYCARDSSRV
GAYLVFDYWGRGTMVTVSSGGGGSGGGGSGGGGSAQSVLTQPPSVSGAPGQRVTISCTGSS
SNIGAGYDVHWYQQLPGTAPKLLIYGNSKRPSGVPDRFSGSKSGTSASLALTGLQAEDEADY
YCQSYDSSLSGYVFGTGTKVTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 608)
>Her139_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacggcctgagagtcgaggacacggccgtgtattactgtgcgaaagaattggtcagtagagggagcctcacctttg
actactggggcaaggggacaatggtcaccgtctcgagtggaggcggcggttcaggcggaggtggctctggcggtggcggaagtgcacagtctg
tgttgacgcagccgccctcagtgtctggggccccagggcagggggtcaccatctcctgcactgggagcagctccaacatcggggcagattttgct
gtacactggtaccaacaacttccagggacagcccccaaactcctcatcaatggtagcagccatcggccctcaggggtccctgaccgattctctggc
tccaagtctggcccctcagcctccctggccatcactgggctccaagccgacgatgaggctgattatttttgccagtcctatgactacagactcaatgct
ttagtgttcggcggagggaccaagctgaccgtcctaggtgacgtacgcgagcccaaatcttctgacaaaactcacacatgcccaccgtgcccagc
acctgaactcctgggtggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaagccgcgggagga
gcagtacaacagcacgtaccgtgtggtcagcgtcctcaccgtcctgcaccaggactggctgaatggcaaggagtacaagtgcaaggtctccaaca
aagccctcccagcccccatcgagaaaaccatctccaaagccaaagggcagccccgagaaccacaggtgtacaccctgcccccatcccgggatg
agctgaccaagaaccaggtcagcctgacctgcctggtcaaaggcttctatccaagcgacatcgccgtggagtgggagagcaatgggcagccgg
agaacaactacaagaccacgcctcccgtgctggactccgacggctccttcttcctctacagcaagctcaccgtggacaagagcaggtggcagcag
gggaacgtcttctcatgctccgtgatgcatgaggctctgcacaaccactacacgcagaagagcctctccctgtctcc
gggtaaatga (SEQ ID NO: 609)
>Her139_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNGLRVEDTAVYYCAKELVS
RGSLTFDYWGKGTMVTVSSGGGGSGGGGSGGGGSAQSVLTQPPSVSGAPGQGVTISCTGSSS
NIGADFAVHWYQQLPGTAPKLLINGSSHRPSGVPDRFSGSKSGPSASLAITGLQADDEADYFC
QSYDYRLNALVFGGGTKLTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYT
QKSLSLSPGK. (SEQ ID NO: 610)
>Her140_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagagatagacggccgagggatgatgcttttgata
tgtgggggagagggaccacggtcaccgtctcgagtggaggcggcggttcaggcggaggtggctctggcggtggcggaagtgcacttaacatcc
agatgacccagtctccctcttctgtgtctgcttctgttggagacagagtcagcatcacttgtcgggcgagtcagggaattggcagctggttattctggta
tcagcagaaaccagggaaagcccctatcctcctgatgtccgctgtgtccggtttgcaaagtggagtcccatcacgattcagcggcagcggatctgg
gacagatttcactctcacgatcagcagcgtacagcctgaggattttgcaacttactattgtcaacaggctcacagtttccctatcaccttcggccaagg
gacacgactggagattaaacgtgacgtacgcgagcccaaatcttctgacaaaactcacacatgcccaccgtgcccagcacctgaactcctgggtg
gaccgtcagtcttcctcttccccccaaaacccaaggacaccctcatgatctcccggacccctgaggtcacatgcgtggtggtggacgtgagccacg
aagaccctgaggtcaagttcaactggtacgtggacggcgtggaggtgcataatgccaagacaaagccgcgggaggagcagtacaacagcacgt
accgtgtggtcagcgtcctcaccgtcctgcaccaggactggctgaatggcaaggagtacaagtgcaaggtctccaacaaagccctcccagccccc
atcgagaaaaccatctccaaagccaaagggcagccccgagaaccacaggtgtacaccctgcccccatcccgggatgagctgaccaagaaccag
gtcagcctgacctgcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaacgtcttctcatgct
ccgtgatgcatgaggctctgcacaaccactacacgcagaagagcctctccctgtctccgggtaaatga (SEQ ID NO: 611)
>Her140_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARDRRP
RDDAFDMWGRGTTVTVSSGGGGSGGGGSGGGGSALNIQMTQSPSSVSASVGDRVSITCRAS
QGIGSWLFWYQQKPGKAPILLMSAVSGLQSGVPSRFSGSGSGTDFTLTISSVQPEDFATYYCQ
QAHSFPITFGQGTRLEIKRDVREPKSSDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSL
SLSPGK. (SEQ ID NO: 612)
>Her141_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactccgcagactccgtgaagggccggttcaccatctccagagacaactccaag
aacacgctgtatctgcaaatgaacagcctgagagccgaggacacggccgtgtattactgtgcgggaggtagagtgggatctacggcggcttttgat
acatgggggcgagggaccacggtcaccgtctcgagtggaggcggcggttcaggcggaggtggctctggcggtggcggaagtgcacaggctgt
gctgactcagccgtcctcagtgtctggggccccagggcagagggtcaccatctcctgcactgggagcagctccaacatcggggcaggttatgatg
tacactggtaccagcagcttccaggaacagcccccaaactcctcatctatggtaacagcaatcggccctcaggggtccctgaccgattctctggctc
caagtctggcacctcagcctccctggccatcactgggctccaggctgaggatgaggctgattattactgccagtcctatgacagcagcctgcgtggt
tatgtcttcggaactgggaccaagctgaccgtcctaggtgacgtacgcgagcccaaatcttctgacaaaactcacacatgcccaccgtgcccagca
cctgaactcctgggtggaccgtcagtcttcctcttccccccaaaacccaaggacaccctcatgatctcccggacccctgaggtcacatgcgtggtgg
tggacgtgagccacgaagaccctgaggtcaagttcaactggtacgtggacggcgtggaggtgcataatgccaagacaaagccgcgggaggagc
agtacaacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccgggatgag
ctgaccaagaaccaggtcagcctgacctgcctggtcaaaggcttctatccaagcgacatcgccgtggagtgggagagcaatgggcagccggag
aacaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 613)
>Her141_Protein_leader-stop
MEAPAQLLFLLLLWLPDTTGEVQLLESGGGLVQPGGSLRLSCAASGFTFSSYAMSWVRQAP
GKGLEWVSAISGSGGSTYSADSVKGRFTISRDNSKNTLYLQMNSLRAEDTAVYYCAGGRVG
STAAFDTWGRGTTVTVSSGGGGSGGGGSGGGGSAQAVLTQPSSVSGAPGQRVTISCTGSSSN
IGAGYDVHWYQQLPGTAPKLLIYGNSNRPSGVPDRFSGSKSGTSASLAITGLQAEDEADYYC
QSYDSSLRGYVFGTGTKLTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 614)
>Her142_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagagatcgagccctagtgggagctactcgaactt
ttggctactgggggcaggggaccacggtcaccgtctcgagtggaggcggcggttcaggcggaggtggctctggcggtggcggaagtgcacag
gctgtgctgactcagccgtcctcagtgtctggggccccagggcagagggtcaccatctcctgcactgggagcagctccaacatcggggcaggtta
tgatgtacactggtaccagcagcttccaggaacagcccccaaactcctcatctatggtgacaccaatcggccctcaggggtccctgaccgattctct
ggctccaagtctggcacctcagcctccctggccatcactgggctccaggctgaggatgaggctgattattactgccagtcctttgacagcagcctca
gtggttcggtgttcggcggagggaccaagctgaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctgaggtcacatgc
gtggtggtggacgtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtacaagtgcaaggtct
ccaacaaagccctcccagcccccatcgagaaaaccatctccaaagccaaagggcagccccgagaaccacaggtgtacaccctgcccccatccc
gggatgagctgaccaagaaccaggtcagcctgacctgcctggtcaaaggcttctatccaagcgacatcgccgtggagtgggagagcaatgggca
gccggagaacaactacaagaccacgcctcccgtgctggactccgacggctccttcttcctctacagcaagctcaccgtggacaagagcaggtggc
agcaggggaacgtcttctcatgctccgtgatgcatgaggctctgcacaaccactacacgcagaagagcctctccctgtctcc
gggtaaatga (SEQ ID NO: 615)
>Her142_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARDRAL
VGATRTFGYWGQGTTVTVSSGGGGSGGGGSGGGGSAQAVLTQPSSVSGAPGQRVTISCTGS
SSNIGAGYDVHWYQQLPGTAPKLLIYGDTNRPSGVPDRFSGSKSGTSASLAITGLQAEDEAD
YYCQSFDSSLSGSVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 616)
>Her143_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagagttgggtcgtttggtgattacaaagataaaag
tggttacggcttctactttgactactggggccaaggcaccctggtcaccgtctcgagtggaggcggcggttcaggcggaggtggctctggcggtg
gcggaagtgcacagtctgtgctgactcagccaccctcggcgtctgggaccctcgggcagacggtcttcatctcttgttctggaagcagttccaacat
cggaagtaattctgtgagttggtaccagcagctcccaggaacggcccccaaatttctcatttatcataataatcagcggccctcaggggtccctgagc
gattctctggctccaagtctggcacctcagcctccctggccatccgtgggctccagtctgaggatgaggctgattactactgtgcatcttgggaggac
agcctgaatggttgggtgttcggcggggggaccaagctgaccgtcctaggtgacgtacgcgagcccaaatcttctgacaaaactcacacatgccc
accgtgcccagcacctgaactcctgggtggaccgtcagtcttcctcttccccccaaaacccaaggacaccctcatgatctcccggacccctgaggt
cacatgcgtggtggtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaaggagtacaagtgc
aaggtctccaacaaagccctcccagcccccatcgagaaaaccatctccaaagccaaagggcagccccgagaaccacaggtgtacaccctgccc
ccatcccgggatgagctgaccaagaaccaggtcagcctgacctgcctggtcaaaggcttctatccaagcgacatcgccgtggagtgggagagca
atgggcagccggagaacaactacaagaccacgcctcccgtgctggactccgacggctccttcttcctctacagcaagctcaccgtggacaagagc
aggtggcagcaggggaacgtcttctcatgctccgtgatgcatgaggctctgcacaaccactacacgcagaagagcctctccctgtctccgggtaaa
tga (SEQ ID NO: 617)
>Her143_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARVGSF
GDYKDKSGYGFYFDYWGQGTLVTVSSGGGGSGGGGSGGGGSAQSVLTQPPSASGTLGQTV
FISCSGSSSNIGSNSVSWYQQLPGTAPKFLIYHNNQRPSGVPERFSGSKSGTSASLAIRGLQSED
EADYYCASWEDSLNGWVFGGGTKLTVLGDVREPKSSDKTHTCPPCPAPELLGGPSVFLFPPK
PKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHE
ALHNHYTQKSLSLSPGK. (SEQ ID NO: 618)
>Her144_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgggaggtagagtgggatctacggcggcttttgata
catggggccaggggacaatggtcaccgtctcgagtggaggcggcggttcaggcggaggtggctctggcggtggcggaagtgcacagtctgtgt
taacgcagccgccctcagtgtctggggccccagggcagagggtcgccatatcctgtacggggagcagctccaatattggggcaggttatgatgta
cactggtttcagcaacttccaggaacagcccccaaactcctcatctttggtaacaagaatcggccctcaggggtccccgaccgattctctgcctctaa
gtctggcaccgcagcctccctggccatcactgggctccaggctgaggatgagggtgattattactgccagtcctatgacagcagcctgagtggtgt
gatcttcggcagagggaccaagctgaccgtcctaggtgacgtacgcgagcccaaatcttctgacaaaactcacacatgcccaccgtgcccagcac
ctgaactcctgggtggaccgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaaagccgcgggaggagc
agtacaacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccgggatgag
ctgaccaagaaccaggtcagcctgacctgcctggtcaaaggcttctatccaagcgacatcgccgtggagtgggagagcaatgggcagccggag
aacaactacaagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaatga (SEQ ID NO: 619)
>Her144_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAGGRVG
STAAFDTWGQGTMVTVSSGGGGSGGGGSGGGGSAQSVLTQPPSVSGAPGQRVAISCTGSSS
NIGAGYDVHWFQQLPGTAPKLLIFGNKNRPSGVPDRFSASKSGTAASLAITGLQAEDEGDYY
CQSYDSSLSGVIFGRGTKLTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYT
QKSLSLSPGK. (SEQ ID NO: 620)
>Her145_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgggagcggcggactacagtaattactttgacttttg
gggccaagggacaatggtcaccgtctcgagtggaggcggcggttcaggcggaggtggctctggcggtggcggaagtgcacagtctgtgctgac
tcagccaccctcagtgtctggggccccagggcagagggtcaccatctcctgcactgggagcagctccaacatcggggcaggttatgatgtacact
ggtaccagcaacttccaggagcagcccccaaactcctcatctatgggaacatcaatcggccctcaggggtccctgaccgattctctggctccaagt
ctggcacctcagcctccctggccatcactgggctccaggctgaggatgagggtgattattactgccagtcctatgacagaagcctgagtgctaagct
gttcggcggagggaccaaggtcaccgtcctaggtgacgtacgcgagcccaaatcttctgacaaaactcacacatgcccaccgtgcccagcacctg
aactcctgggtggaccgtcagtcttcctcttccccccaaaacccaaggacaccctcatgatctcccggacccctgaggtcacatgcgtggtggtgga
cgtgagccacgaagaccctgaggtcaagttcaactggtacgtggacggcgtggaggtgcataatgccaagacaaagccgcgggaggagcagta
caacagcacgtaccgtgtggtcagcgtcctcaccgtcctgcaccaggactggctgaatggcaaggagtacaagtgcaaggtctccaacaaagcc
ctcccagcccccatcgagaaaaccatctccaaagccaaagggcagccccgagaaccacaggtgtacaccctgcccccatcccgggatgagctg
accaagaaccaggtcagcctgacctgcctggtcaaaggcttctatccaagcgacatcgccgtggagtgggagagcaatgggcagccggagaac
aactacaagaccacgcctcccgtgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtaaatga (SEQ ID NO: 621)
>Her145_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAGAADY
SNYFDFWGQGTMVTVSSGGGGSGGGGSGGGGSAQSVLTQPPSVSGAPGQRVTISCTGSSSNI
GAGYDVHWYQQLPGAAPKLLIYGNINRPSGVPDRFSGSKSGTSASLAITGLQAEDEGDYYCQ
SYDRSLSAKLFGGGTKVTVLGDVREPKSSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQ
KSLSLSPGK. (SEQ ID NO: 622)
>Her146_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcggcgagtaatagttattactactttgactactggggc
cagggaaccctggtcaccgtctcgagtggaggcggcggttcaggcggaggtggctctggcggtggcggaagtgcactttcttctgagctgactca
ggaccctgctgtgtctgtggccttgggacagacagtcaggatcacatgccaaggagacagcctcagaaacttttatccaagttggtatcagcagaa
gccaggacaggcccctgttcttgtcatttatggtaaaaatattcggccctcagggatcccagaccgattctctggctccggctcaggaagcacagctt
ccttgaccatcactggggctcaggcggaagatgaggctgactattactgtaactcccgggacagcagtggtaaacatatgggggtggtattcggcg
gagggaccaagctgaccgtcctaggtgacgtacgcgagcccaaatcttctgacaaaactcacacatgcccaccgtgcccagcacctgaactcctg
ggtggaccgtcagtcttcctcttccccccaaaacccaaggacaccctcatgatctcccggacccctgaggtcacatgcgtggtggtggacgtgagc
cacgaagaccctgaggtcaagttcaactggtacgtggacggcgtggaggtgcataatgccaagacaaagccgcgggaggagcagtacaacagc
acgtaccgtgtggtcagcgtcctcaccgtcctgcaccaggactggctgaatggcaaggagtacaagtgcaaggtctccaacaaagccctcccagc
ccccatcgagaaaaccatctccaaagccaaagggcagccccgagaaccacaggtgtacaccctgcccccatcccgggatgagctgaccaagaa
ccaggtcagcctgacctgcctggtcaaaggcttctatccaagcgacatcgccgtggagtgggagagcaatgggcagccggagaacaactacaag
accacgcctcccgtgctggactccgacggctccttcttcctctacagcaagctcaccgtggacaagagcaggtggcagcaggggaacgtcttctca
tgctccgtgatgcatgaggctctgcacaaccactacacgcagaagagcctctccctgtctccgggtaaatga (SEQ ID NO: 623)
>Her146_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAASNSY
YYFDYWGQGTLVTVSSGGGGSGGGGSGGGGSALSSELTQDPAVSVALGQTVRITCQGDSLR
NFYPSWYQQKPGQAPVLVIYGKNIRPSGIPDRFSGSGSGSTASLTITGAQAEDEADYYCNSRD
SSGKHMGVVFGGGTKLTVLGDVREPKSSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQ
KSLSLSPGK. (SEQ ID NO: 624)
>Her147_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acgcgctgtatctgcaaatgaacagcctgagagccgaggacacggccgtgtattactgtgcgagagatctgggaatagaccccctttggagtggtt
attacacaccccttgactattggggccgggggacaatggtcaccgtctcgagtggaggcggcggttcaggcggaggtggctctggcggtggcgg
aagtgcactttcttctgagctgactcaggaccctgctctgtcggtggccttgggacagacagtcaggatcacatgtcaaggggacagcctcggagg
ctttcatgcaagctggtaccaggagaagccaggacaggcccctgtatttgtcctctatggtaaaaacaaccggccctcagggatcccagaccgattc
tctggctccacctcaggtaacacagctgccctgaccatcactggggctcaggcggaagatgaggctgactattactgtagctcccgggacagaagt
ggtaaccatcgcgtcttcggcggagggaccaagctgaccgtcctaggtgacgtacgcgagcccaaatcttctgacaaaactcacacatgcccacc
gtgcccagcacctgaactcctgggtggaccgtcagtcttcctcttccccccaaaacccaaggacaccctcatgatctcccggacccctgaggtcac
atgcgtggtggtggacgtgagccacgaagaccctgaggtcaagttcaactggtacgtggacggcgtggaggtgcataatgccaagacaaagccg
cgggaggagcagtacaacagcacgtaccgtgtggtcagcgtcctcaccgtcctgcaccaggactggctgaatggcaaggagtacaagtgcaag
gtctccaacaaagccctcccagcccccatcgagaaaaccatctccaaagccaaagggcagccccgagaaccacaggtgtacaccctgcccccat
cccgggatgagctgaccaagaaccaggtcagcctgacctgcctggtcaaaggcttctatccaagcgacatcgccgtggagtgggagagcaatgg
gcagccggagaacaactacaagaccacgcctcccgtgctggactccgacggctccttcttcctctacagcaagctcaccgtggacaagagcaggt
ggcagcaggggaacgtcttctcatgctccgtgatgcatgaggctctgcacaaccactacacgcagaagagcctctccctgtctcc
gggtaaatga (SEQ ID NO: 625)
>Her147_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNALYLQMNSLRAEDTAVYYCARDLGI
DPLWSGYYTPLDYWGRGTMVTVSSGGGGSGGGGSGGGGSALSSELTQDPALSVALGQTVRI
TCQGDSLGGFHASWYQEKPGQAPVFVLYGKNNRPSGIPDRFSGSTSGNTAALTITGAQAEDE
ADYYCSSRDRSGNHRVFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK. (SEQ ID NO: 626)
>Her148_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgaggggctacagtggaagttcctttgacgcctggg
gccaagggacaatggtcaccgtctcgagtggaggcggcggttcaggcggaggtggctctggcggtggcggaagtgcacagtctgtgttgacgc
agccgccatcagcgtccgggacccccgggcagagggtcaccatctcttgttctggaagcagctccaatatcggaagtaagtctgtatactggtacc
agcaactcccaggagcggcccccaaactcctcatctacaggaatagtcagcggccctcaggggtccctgaccgattctctgcctccaagtctggca
cctctgcctccctggccatcagtgggctccggtccgaggatgaggctgactattactgtgcagcatgggatggcagcctgagtggacatttcttcgg
aactgggaccaagctgaccgtcctaggtgacgtacgcgagcccaaatcttctgacaaaactcacacatgcccaccgtgcccagcacctgaactcct
gggtggaccgtcagtcttcctcttccccccaaaacccaaggacaccctcatgatctcccggacccctgaggtcacatgcgtggtggtggacgtgag
ccacgaagaccctgaggtcaagttcaactggtacgtggacggcgtggaggtgcataatgccaagacaaagccgcgggaggagcagtacaacag
cacgtaccgtgtggtcagcgtcctcaccgtcctgcaccaggactggctgaatggcaaggagtacaagtgcaaggtctccaacaaagccctcccag
cccccatcgagaaaaccatctccaaagccaaagggcagccccgagaaccacaggtgtacaccctgcccccatcccgggatgagctgaccaaga
accaggtcagcctgacctgcctggtcaaaggcttctatccaagcgacatcgccgtggagtgggagagcaatgggcagccggagaacaactacaa
gaccacgcctcccgtgctggactccgacggctccttcttcctctacagcaagctcaccgtggacaagagcaggtggcagcaggggaacgtcttct
catgctccgtgatgcatgaggctctgcacaaccactacacgcagaagagcctctccctgtctccgggtaaatga (SEQ ID NO: 627)
>Her148_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARGYSG
SSFDAWGQGTMVTVSSGGGGSGGGGSGGGGSAQSVLTQPPSASGTPGQRVTISCSGSSSNIGS
KSVYWYQQLPGAAPKLLIYRNSQRPSGVPDRFSASKSGTSASLAISGLRSEDEADYYCAAWD
GSLSGHFFGTGTKLTVLGDVREPKSSDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSL
SLSPGK. (SEQ ID NO: 628)
>Her149_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgggagaggcagcagagtggggacgatttggggaag
ccttgacttttggggccaagggacaatggtcaccgtctcgagtggaggcggcggttcaggcggaggtggctctggcggtggcggaagtgcacag
tctgtgctgacgcagccgccctcagtgtctgcggccccgggacagagggtcaccatctcctgctctggaaagagctccaacattggcggtaattct
gtggcctggtaccagcaactcccgggaacagcccccaaagtcctcatttatgacaatgataagcgaccctcaggggttcctgaccgattctctggct
ccaagtctggcacgtcagccaccctgggcatcaccggactgcagactggggacgaggccgattattactgcggatcctgggatagcagcctggg
tgttgggatgttcggcggagggaccaaggtcaccgtcctaggtgacgtacgcgagcccaaatcttctgacaaaactcacacatgcccaccgtgcc
cagcacctgaactcctgggtggaccgtcagtcttcctcttccccccaaaacccaaggacaccctcatgatctcccggacccctgaggtcacatgcgt
ggtggtggacgtgagccacgaagaccctgaggtcaagttcaactggtacgtggacggcgtggaggtgcataatgccaagacaaagccgcggga
ggagcagtacaacagcacgtaccgtgtggtcagcgtcctcaccgtcctgcaccaggactggctgaatggcaaggagtacaagtgcaaggtctcc
aacaaagccctcccagcccccatcgagaaaaccatctccaaagccaaagggcagccccgagaaccacaggtgtacaccctgcccccatcccgg
gatgagctgaccaagaaccaggtcagcctgacctgcctggtcaaaggcttctatccaagcgacatcgccgtggagtgggagagcaatgggcagc
cggagaacaactacaagaccacgcctcccgtgctggactccgacggctccttcttcctctacagcaagctcaccgtggacaagagcaggtggcag
caggggaacgtcttctcatgctccgtgatgcatgaggctctgcacaaccactacacgcagaagagcctctccctgtctcc
gggtaaatga (SEQ ID NO: 629)
>Her149_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCGRGSRV
GTIWGSLDFWGQGTMVTVSSGGGGSGGGGSGGGGSAQSVLTQPPSVSAAPGQRVTISCSGK
SSNIGGNSVAWYQQLPGTAPKVLIYDNDKRPSGVPDRFSGSKSGTSATLGITGLQTGDEADY
YCGSWDSSLGVGMFGGGTKVTVLGDVREPKSSDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 630)
>Her150_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagagatcgcgtttacgatttttggagtggttattata
cgaggtacaactggttcgacccctgggggcgagggaccacggtcaccgtctcgagtggaggcggcggttcaggcggaggtggctctggcggt
ggcggaagtgcacaggctgtgctgactcagccgtcctcagcgtctgggacccccgggcagagggtcaccatctcttgttctggaagcagctccaa
catcggaagtaattatgtatactggtaccagcagctcccaggaacggcccccaaactcctcatctataggaataatcagcggccctcaggggtccct
gaccgattctctggctccaagtctggcacctcagcctccctggccatcagtgggctccggtccgaggatgaggctgattattactgtgcagcatggg
atgacagcctgagtagtccggtgttcggcggagggaccaaggtcaccgtcctaggtgacgtacgcgagcccaaatcttctgacaaaactcacaca
tgcccaccgtgcccagcacctgaactcctgggtggaccgtcagtcttcctcttccccccaaaacccaaggacaccctcatgatctcccggacccct
gaggtcacatgcgtggtggtggacgtgagccacgaagaccctgaggtcaagttcaactggtacgtggacggcgtggaggtgcataatgccaaga
caaagccgcgggaggagcagtacaacagcacgtaccgtgtggtcagcgtcctcaccgtcctgcaccaggactggctgaatggcaaggagtaca
agtgcaaggtctccaacaaagccctcccagcccccatcgagaaaaccatctccaaagccaaagggcagccccgagaaccacaggtgtacaccc
tgcccccatcccgggatgagctgaccaagaaccaggtcagcctgacctgcctggtcaaaggcttctatccaagcgacatcgccgtggagtggga
gagcaatgggcagccggagaacaactacaagaccacgcctcccgtgctggactccgacggctccttcttcctctacagcaagctcaccgtggaca
agagcaggtggcagcaggggaacgtcttctcatgctccgtgatgcatgaggctctgcacaaccactacacgcagaagagcctctccctgtctccg
ggtaaatga (SEQ ID NO: 631)
>Her150_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARDRVY
DFWSGYYTRYNWFDPWGRGTTVTVSSGGGGSGGGGSGGGGSAQAVLTQPSSASGTPGQRV
TISCSGSSSNIGSNYVYWYQQLPGTAPKLLIYRNNQRPSGVPDRFSGSKSGTSASLAISGLRSE
DEADYYCAAWDDSLSSPVFGGGTKVTVLGDVREPKSSDKTHTCPPCPAPELLGGPSVFLFPP
KPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMH
EALHNHYTQKSLSLSPGK. (SEQ ID NO: 632)
>Her151_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccagactccagggaaggggc
tggagtgggtctcagctattagtggtagtggtggtagcacatactacgcaaactccgtgaagggccggttcaccatctccagagacaattccaagaa
cacgctgtatctgcaaatgaacagcctgagagccgaggacacggccgtgtattactgtgcgcgggggggaggctacaacccttttgactcctggg
gccaggggacaatggtcaccgtctcgagtggaggcggcggttcaggcggaggtggctctggcggtggcggaagtgcacagtctgccctgactc
agcctgcctccgtgtctgggtctcctggacagtcgatcaccatctcctgcactggaaccggcagtgacgttggtggttataactatgtctcctggtacc
aacagcacccaggcaaagcccccaaactcatgatttatgaggtcattaatcggccctcagggatttctaatcgcttctctggctccaagtctggcaac
acggcctccctgaccatctctgggctccaggctgaggacgaggctgattattactgcggctcatattcaagcagcagcactcttgtattcggcggag
ggaccaagctgaccgtcctaggtgacgtacgcgagcccaaatcttctgacaaaactcacacatgcccaccgtgcccagcacctgaactcctgggt
ggaccgtcagtcttcctcttccccccaaaacccaaggacaccctcatgatctcccggacccctgaggtcacatgcgtggtggtggacgtgagccac
gaagaccctgaggtcaagttcaactggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgcaaggtctccaacaaagccctcccagccc
ccatcgagaaaaccatctccaaagccaaagggcagccccgagaaccacaggtgtacaccctgcccccatcccgggatgagctgaccaagaacc
aggtcagcctgacctgcctggtcaaaggcttctatccaagcgacatcgccgtggagtgggagagcaatgggcagccggagaacaactacaagac
cacgcctcccgtgctggactccgacggctccttcttcctctacagcaagctcaccgtggacaagagcaggtggcagcaggggaacgtcttctcatg
ctccgtgatgcatgaggctctgcacaaccactacacgcagaagagcctctccctgtctccgggtaaatga (SEQ ID NO: 633)
>Her151_Protein_leader-stop
MEAPAQLLFLLLLWLPDTTGEVQLLESGGGLVQPGGSLRLSCAASGFTFSSYAMSWVRQTPG
KGLEWVSAISGSGGSTYYANSVKGRFTISRDNSKNTLYLQMNSLRAEDTAVYYCARGGGYN
PFDSWGQGTMVTVSSGGGGSGGGGSGGGGSAQSALTQPASVSGSPGQSITISCTGTGSDVGG
YNYVSWYQQHPGKAPKLMIYEVINRPSGISNRFSGSKSGNTASLTISGLQAEDEADYYCGSYS
SSSTLVFGGGTKLTVLGDVREPKSSDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLS
LSPGK. (SEQ ID NO: 634)
>Her152_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagaggtcacaaaatgggatactttgactactggg
gccggggcaccctggtcaccgtctcgagtggaggcggcggttcaggcggaggtggctctggcggtggcggaagtgcactttcttctgagctgact
caggaccctgctgtgtccgtggccttgggacagtcagtcaccatcacgtgtcggggagccagcctcagcaactattatgcaagctggtaccagcag
aggccaggacaagcccctctacttgtcgtctctgataacaacatccggccctcagggatcccagaccgattctctggctccaggtcaggaaccaca
gcttccttgagcatcactggggctcaggcggaagatgaggctgactattactgtcactcccgtgccagcagtgacacccatgtccgggtgtttggcg
gcgggaccaagctgaccgtcctaggtgacgtacgcgagcccaaatcttctgacaaaactcacacatgcccaccgtgcccagcacctgaactcctg
ggtggaccgtcagtcttcctcttccccccaaaacccaaggacaccctcatgatctcccggacccctgaggtcacatgcgtggtggtggacgtgagc
cacgaagaccctgaggtcaagttcaactggtacgtggacggcgtggaggtgcataatgccaagacaaagccgcgggaggagcagtacaacagc
acgtaccgtgtggtcagcgtcctcaccgtcctgcaccaggactggctgaatggcaaggagtacaagtgcaaggtctccaacaaagccctcccagc
ccccatcgagaaaaccatctccaaagccaaagggcagccccgagaaccacaggtgtacaccctgcccccatcccgggatgagctgaccaagaa
ccaggtcagcctgacctgcctggtcaaaggcttctatccaagcgacatcgccgtggagtgggagagcaatgggcagccggagaacaactacaag
accacgcctcccgtgctggactccgacggctccttcttcctctacagcaagctcaccgtggacaagagcaggtggcagcaggggaacgtcttctca
tgctccgtgatgcatgaggctctgcacaaccactacacgcagaagagcctctccctgtctccgggtaaatga (SEQ ID NO: 635)
>Her152_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARGHKM
GYFDYWGRGTLVTVSSGGGGSGGGGSGGGGSALSSELTQDPAVSVALGQSVTITCRGASLS
NYYASWYQQRPGQAPLLVVSDNNIRPSGIPDRFSGSRSGTTASLSITGAQAEDEADYYCHSRA
SSDTHVRVFGGGTKLTVLGDVREPKSSDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 636)
>Her153_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagggacagggtctctaactggaactactacggc
caggacagctactttgactactggggccaggggacaatggtcaccgtctcgagtggaggcggcggttcaggcggaggtggctctggcggtggc
ggaagtgcacagtctgtgctgactcagccaccctccgcgtctggggcccccgggcagagggtcaccatttcttgttctgggaccaactccaacatc
ggaagtaataatgtaaactggtatcagcaactcccaggaaaggcccccagactcctcatctacaataataatcagaggccctcaggggtccctgac
cgattctctggctccaagtctggcacctcagcctccctggccatcagtgggctccagtctgagcttgaggctgattattattgttcagcatgggatgac
agcctgcatggtccggtgttcggcggagggaccaagctgaccgtcctaggtgacgtacgcgagcccaaatcttctgacaaaactcacacatgccc
accgtgcccagcacctgaactcctgggtggaccgtcagtcttcctcttccccccaaaacccaaggacaccctcatgatctcccggacccctgaggt
cacatgcgtggtggtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaaggagtacaagtgc
aaggtctccaacaaagccctcccagcccccatcgagaaaaccatctccaaagccaaagggcagccccgagaaccacaggtgtacaccctgccc
ccatcccgggatgagctgaccaagaaccaggtcagcctgacctgcctggtcaaaggcttctatccaagcgacatcgccgtggagtgggagagca
atgggcagccggagaacaactacaagaccacgcctcccgtgctggactccgacggctccttcttcctctacagcaagctcaccgtggacaagagc
aggtggcagcaggggaacgtcttctcatgctccgtgatgcatgaggctctgcacaaccactacacgcagaagagcctctccctgtctccgggtaaa
tga (SEQ ID NO: 637)
>Her153_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARDRVS
NWNYYGQDSYFDYWGQGTMVTVSSGGGGSGGGGSGGGGSAQSVLTQPPSASGAPGQRVTI
SCSGTNSNIGSNNVNWYQQLPGKAPRLLIYNNNQRPSGVPDRFSGSKSGTSASLAISGLQSEL
EADYYCSAWDDSLHGPVFGGGTKLTVLGDVREPKSSDKTHTCPPCPAPELLGGPSVFLFPPK
PKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHE
ALHNHYTQKSLSLSPGK. (SEQ ID NO: 638)
>Her154_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagggacagggtctctaactggaactactacggc
caggacagctactttggctactggggccaggggacaatggtcaccgtctcgagtggaggcggcggttcaggcggaggtggctctggcggtggc
ggaagtgcactttcctatgagctgactcagccaccctcagcgtctgggacccccgggcagagggtcaccatctcttgttctggaagcagctccaac
atcggaagtaatactgtaacctggtaccagcagctcccaggaacggccccccaactcctcttccataataatgaccagcggccctcaggggtccct
gaccgattctctggctccaagtctggcacctcaggctccctggccatcagtgggctgcagtctgaggatgaggctgattattactgttcagcatggga
tgacggcctgaatgctgtaatattcggcggagggaccaagctgaccgtcctaggtgacgtacgcgagcccaaatcttctgacaaaactcacacatg
cccaccgtgcccagcacctgaactcctgggtggaccgtcagtcttcctcttccccccaaaacccaaggacaccctcatgatctcccggacccctga
ggtcacatgcgtggtggtggacgtgagccacgaagaccctgaggtcaagttcaactggtacgtggacggcgtggaggtgcataatgccaagaca
aagccgcgggaggagcagtacaacagcacgtaccgtgtggtcagcgtcctcaccgtcctgcaccaggactggctgaatggcaaggagtacaag
tgcaaggtctccaacaaagccctcccagcccccatcgagaaaaccatctccaaagccaaagggcagccccgagaaccacaggtgtacaccctg
cccccatcccgggatgagctgaccaagaaccaggtcagcctgacctgcctggtcaaaggcttctatccaagcgacatcgccgtggagtgggaga
gcaatgggcagccggagaacaactacaagaccacgcctcccgtgctggactccgacggctccttcttcctctacagcaagctcaccgtggacaag
agcaggtggcagcaggggaacgtcttctcatgctccgtgatgcatgaggctctgcacaaccactacacgcagaagagcctctccctgtctccgggt
aaatga (SEQ ID NO: 639)
>Her154_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARDRVS
NWNYYGQDSYFGYWGQGTMVTVSSGGGGSGGGGSGGGGSALSYELTQPPSASGTPGQRVT
ISCSGSSSNIGSNTVTWYQQLPGTAPQLLFHNNDQRPSGVPDRFSGSKSGTSGSLAISGLQSED
EADYYCSAWDDGLNAVIFGGGTKLTVLGDVREPKSSDKTHTCPPCPAPELLGGPSVFLFPPKP
KDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK. (SEQ ID NO: 640)
>Her156_CDS
atggaagcaccagcgcagcttctcttcctcctgctactctggctcccagataccaccggtgaggtgcagctggtgcagtctggggcagaggtgaaa
aagcccggggagtctctgaagatctcctgtaagggttctgggtacagctttagtaactactggatcggctgggtgcgccagatgcccgggaaaggc
ctggagtggatggggatcatctatcctggtgactctgataccagatacagcccgtccttccaaggccaggtcaccatctcagccgacaagtccatca
gtaccgcctacctgcagtggagcagcctgaaggcctcggacagtgccatgtattactgtgcgagacatgattcgactatgggatatgatgcttttcat
atgtggggccaaggaaccctggtcaccgtctcgagtggaggcggcggttcaggcggaggtggctctggcggtggcggaagtgcacaggctgtg
ctgactcagccgtcctcagtgtctggggccccagggcagagggtcaccatctcctgcactgggagcagctccaacatcggggcaggttatgatgt
acactggtaccagcagcttccaggaacagcccccaaactcctcatctatggtaacagcaatcggccctcaggggtccctgaccgattctctggctcc
aagtctggcacctcagcctccctggccatcactgggctccaggctgaggatgaggctgattattactgccagtcctatgacagcagcctgagtggc
cctgtggtattcggcggagggaccaagctgaccgtcctaggtgacgtacgcgagcccaaatcttctgacaaaactcacacatgcccaccgtgccc
agcacctgaactcctgggtggaccgtcagtcttcctcttccccccaaaacccaaggacaccctcatgatctcccggacccctgaggtcacatgcgtg
gtggtggacgtgagccacgaagaccctgaggtcaagttcaactggtacgtggacggcgtggaggtgcataatgccaagacaaagccgcgggag
gagcagtacaacagcacgtaccgtgtggtcagcgtcctcaccgtcctgcaccaggactggctgaatggcaaggagtacaagtgcaaggtctccaa
caaagccctcccagcccccatcgagaaaaccatctccaaagccaaagggcagccccgagaaccacaggtgtacaccctgcccccatcccggga
tgagctgaccaagaaccaggtcagcctgacctgcctggtcaaaggcttctatccaagcgacatcgccgtggagtgggagagcaatgggcagccg
gagaacaactacaagaccacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtc
tccgggtaaatga (SEQ ID NO: 641)
>Her156_Protein_leader-stop
MEAPAQLLFLLLLWLPDTTGEVQLVQSGAEVKKPGESLKISCKGSGYSFSNYWIGWVRQMP
GKGLEWMGIIYPGDSDTRYSPSFQGQVTISADKSISTAYLQWSSLKASDSAMYYCARHDSTM
GYDAFHMWGQGTLVTVSSGGGGSGGGGSGGGGSAQAVLTQPSSVSGAPGQRVTISCTGSSS
NIGAGYDVHWYQQLPGTAPKLLIYGNSNRPSGVPDRFSGSKSGTSASLAITGLQAEDEADYY
CQSYDSSLSGPVVFGGGTKLTVLGDVREPKSSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 642)
>Her157_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagataattccaaga
acacgctgtatctgcaaatgaacagcctgagagccgaggacacggccgtgtattactgtgcgagagtgggggccggggagaactactaccacta
ctacatcatggacgtctggggccggggcaccctggtcaccgtctcgagtggaggcggcggttcaggcggaggtggctctggcggtggcggaag
tgcactttcttctgagctgactcaggaccctgctgtgtctgtggccttgggacagagagtcagggtcacatgccaaggagacagcctcagaggctat
tatgcaagctggtaccagcagaagccaggacaggcccctgttcttgtcatctatggtgaaaacaaccggccctcagggatcccagaccgattctct
ggctccagctcaggaaacacagcttccttgaccatcactggggctcaggcggaagatgaggctgactattactgtaactcccggcacagcagtggt
aattatctggtgttcggcggagggaccaagctgaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctgaggtcacatgc
gtggtggtggacgtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtacaagtgcaaggtct
ccaacaaagccctcccagcccccatcgagaaaaccatctccaaagccaaagggcagccccgagaaccacaggtgtacaccctgcccccatccc
gggatgagctgaccaagaaccaggtcagcctgacctgcctggtcaaaggcttctatccaagcgacatcgccgtggagtgggagagcaatgggca
gccggagaacaactacaagaccacgcctcccgtgctggactccgacggctccttcttcctctacagcaagctcaccgtggacaagagcaggtggc
agcaggggaacgtcttctcatgctccgtgatgcatgaggctctgcacaaccactacacgcagaagagcctctccctgtctc
cgggtaaatga (SEQ ID NO: 643)
>Her157_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARVGAG
ENYYHYYIMDVWGRGTLVTVSSGGGGSGGGGSGGGGSALSSELTQDPAVSVALGQRVRVT
CQGDSLRGYYASWYQQKPGQAPVLVIYGENNRPSGIPDRFSGSSSGNTASLTITGAQAEDEA
DYYCNSRHSSGNYLVFGGGTKLTVLGDVREPKSSDKTHTCPPCPAPELLGGPSVFLFPPKPKD
TLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALH
NHYTQKSLSLSPGK. (SEQ ID NO: 644)
>Her158_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagaactacggcagatgcttttgatatctggggca
gagggacaatggtcaccgtctcgagtggaggcggcggttcaggcggaggtggctctggcggtggcggaagtgcactttcttctgagctgactcag
gaccctgctgtgtctgtggccttgggacagacagtcaagatcacatgccaaggcgacagcctcagaaactattatgcaggctggtaccagcagaa
gccaggacaggcccctgtacttgtcatctatggtgaaaacaagcggccctcagggatccctgaccgattctctggctccaactcaggaaacacagc
ttccttgaccctcactggggctcaggcggaagatgaggctgactattactgtaactcccgggacagcagtagtaacctcgtggtattcggcggagg
gaccaagctgaccgtcctaggtgacgtacgcgagcccaaatcttctgacaaaactcacacatgcccaccgtgcccagcacctgaactcctgggtg
gaccgtcagtcttcctcttccccccaaaacccaaggacaccctcatgatctcccggacccctgaggtcacatgcgtggtggtggacgtgagccacg
aagaccctgaggtcaagttcaactggtacgtggacggcgtggaggtgcataatgccaagacaaagccgcgggaggagcagtacaacagcacgt
accgtgtggtcagcgtcctcaccgtcctgcaccaggactggctgaatggcaaggagtacaagtgcaaggtctccaacaaagccctcccagccccc
atcgagaaaaccatctccaaagccaaagggcagccccgagaaccacaggtgtacaccctgcccccatcccgggatgagctgaccaagaaccag
gtcagcctgacctgcctggtcaaaggcttctatccaagcgacatcgccgtggagtgggagagcaatgggcagccggagaacaactacaagacca
cgcctcccgtgctggactccgacggctccttcttcctctacagcaagctcaccgtggacaagagcaggtggcagcaggggaacgtcttctcatgct
ccgtgatgcatgaggctctgcacaaccactacacgcagaagagcctctccctgtctccgggtaaatga (SEQ ID NO: 645)
>Her158_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARTTAD
AFDIWGRGTMVTVSSGGGGSGGGGSGGGGSALSSELTQDPAVSVALGQTVKITCQGDSLRN
YYAGWYQQKPGQAPVLVIYGENKRPSGIPDRFSGSNSGNTASLTLTGAQAEDEADYYCNSR
DSSSNLVVFGGGTKLTVLGDVREPKSSDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 646)
>Her159_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctataccatgagctgggtccgccaggctccagggaaggggc
tggagtgggtctcagctattagtggtagtggtggtagcacatactacgcagactccgtgaagggccggttcaccatctccagagacaattccaagaa
cacgctgtatctgcaaatgaacagcctgagagccgaggacacggccgtgtattactgtgcaaggaatagcagcaattggagtggtgcttttgatatc
tgggggcgggggaccacggtcaccgtctcgagtggaggcggcggttcaggcggaggtggctctggcggtggcggaagtgcacagtctgtgct
gacgcagccgccctcagtgtctggggccccagggcagagggtcaccatctcctgcattggaacccactcaaacatcggggcaggttacgctgtg
aactggtaccagcagcttccaggaacagcccccaaactcctcatctatggtaataacaatcggccctcaggggtccctgaccgattctctggctcca
agtctggcacctcagcctccctggccatcaatgggctccaggctgacgatgagtctgattattattgccagtcctatgacgccagtctgagagttttatt
cggcggagggaccaagctgaccgtcctaggtgacgtacgcgagcccaaatcttctgacaaaactcacacatgcccaccgtgcccagcacctgaa
ctcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 647)
>Her159_Protein_leader-stop
MEAPAQLLFLLLLWLPDTTGEVQLLESGGGLVQPGGSLRLSCAASGFTFSSYTMSWVRQAPG
KGLEWVSAISGSGGSTYYADSVKGRFTISRDNSKNTLYLQMNSLRAEDTAVYYCARNSSNW
SGAFDIWGRGTTVTVSSGGGGSGGGGSGGGGSAQSVLTQPPSVSGAPGQRVTISCIGTHSNIG
AGYAVNWYQQLPGTAPKLLIYGNNNRPSGVPDRFSGSKSGTSASLAINGLQADDESDYYCQS
YDASLRVLFGGGTKLTVLGDVREPKSSDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 648)
>Her160_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagagatgacgatttttggagtggttatccattcctc
tactactactacggtatggacgtctggggccgagggacaatggtcaccgtctcgagtggaggcggcggttcaggcggaggtggctctggcggtg
gcggaagtgcacagtctgtcgtgacgcagccgccctcagcgtctgggacccccgggcagagggtcaccatctcttgttctggaactagttccaaca
tcggaagtaatgctgtaaactggtaccagcaactcccaggaacggcccccaaactcctcatctataataataatcagcggccctcaggggtccctga
ccgattctctggctccaagtctggcacctcagcctccctggccatcagtggactccagtctgaggatgaggctgattattactgtgcagcatgggatg
acagcctgaatgtttatgtggtattcggcggagggaccaagctgaccgtcctaggtgacgtacgcgagcccaaatcttctgacaaaactcacacatg
cccaccgtgcccagcacctgaactcctgggtggaccgtcagtcttcctcttccccccaaaacccaaggacaccctcatgatctcccggacccctga
ggtcacatgcgtggtggtggacgtgagccacgaagaccctgaggtcaagttcaactggtacgtggacggcgtggaggtgcataatgccaagaca
aagccgcgggaggagcagtacaacagcacgtaccgtgtggtcagcgtcctcaccgtcctgcaccaggactggctgaatggcaaggagtacaag
tgcaaggtctccaacaaagccctcccagcccccatcgagaaaaccatctccaaagccaaagggcagccccgagaaccacaggtgtacaccctg
cccccatcccgggatgagctgaccaagaaccaggtcagcctgacctgcctggtcaaaggcttctatccaagcgacatcgccgtggagtgggaga
gcaatgggcagccggagaacaactacaagaccacgcctcccgtgctggactccgacggctccttcttcctctacagcaagctcaccgtggacaag
agcaggtggcagcaggggaacgtcttctcatgctccgtgatgcatgaggctctgcacaaccactacacgcagaagagcctctccctgtctccgggt
aaatga (SEQ ID NO: 649)
>Her160_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARDDDF
WSGYPFLYYYYGMDVWGRGTMVTVSSGGGGSGGGGSGGGGSAQSVVTQPPSASGTPGQR
VTISCSGTSSNIGSNAVNWYQQLPGTAPKLLIYNNNQRPSGVPDRFSGSKSGTSASLAISGLQS
EDEADYYCAAWDDSLNVYVVFGGGTKLTVLGDVREPKSSDKTHTCPPCPAPELLGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK. (SEQ ID NO: 650)
>Her161_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacttactacgcagactccgtgaagggccggttcaccatctccagagacaattccaaga
acacgctgtatctgcaaatgaacagcctgagagccgaggacacggccgtgtattactgtgctaaatggggctgggattactatgacacaactggtc
atgatgcctttgatttctggggccgggggacaatggtcaccgtctcgagtggaggcggcggttcaggcggaggtggctctggcggtggcggaagt
gcacaggctgtgctgactcagccgtcctcagtgtctggggccccagggcagagggtcaccatctcctgcactgggagcagctccaacatcgggg
cagattattatgtaaattggtatcagcaacttccaggaaaagcccccgaaatcgtaatttttaatgatgacaatcggccctcaggggtccctaaccgatt
ctctggctccaagtctggcacctcagcctccctggccatcactgggctccaggctgaagatgaggctgattattattgccagtcttatgacagtgtcct
gagtgcttatgtcttcggaactgggaccaaggtcaccgtcctaggtgacgtacgcgagcccaaatcttctgacaaaactcacacatgcccaccgtgc
ccagcacctgaactcctgggtggaccgtcagtcttcctcttccccccaaaacccaaggacaccctcatgatctcccggacccctgaggtcacatgc
gtggtggtggacgtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtacaagtgcaaggtct
ccaacaaagccctcccagcccccatcgagaaaaccatctccaaagccaaagggcagccccgagaaccacaggtgtacaccctgcccccatccc
gggatgagctgaccaagaaccaggtcagcctgacctgcctggtcaaaggcttctatccaagcgacatcgccgtggagtgggagagcaatgggca
gccggagaacaactacaagaccacgcctcccgtgctggactccgacggctccttcttcctctacagcaagctcaccgtggacaagagcaggtggc
agcaggggaacgtcttctcatgctccgtgatgcatgaggctctgcacaaccactacacgcagaagagcctctccctgtctccgggta
aatga (SEQ ID NO: 651)
>Her161_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAKWGW
DYYDTTGHDAFDFWGRGTMVTVSSGGGGSGGGGSGGGGSAQAVLTQPSSVSGAPGQRVTIS
CTGSSSNIGADYYVNWYQQLPGKAPEIVIFNDDNRPSGVPNRFSGSKSGTSASLAITGLQAED
EADYYCQSYDSVLSAYVFGTGTKVTVLGDVREPKSSDKTHTCPPCPAPELLGGPSVFLFPPKP
KDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK. (SEQ ID NO: 652)
>Her162_CDS
atggaagcaccagcgcagcttctcttcctcctgctactctggctcccagataccaccggtgaggtgcagctggtggagactggggctgaggtgaag
aagcctggggcctcaatgaaggtctcctgcaaggcttctggatacagcttcaccgactactatatgcactgggtgcgacaggcccctggacaaggg
cttgagtggatgggatggatcaaccctaatagtggtgacacaaactatgcacagaagtttcagggcagggtcaccatgaccagggacacgtccatc
accacagcctacatggagctgagcaggctgagatctgacgacacggccgtatattactgtgcgacagagaggtataacagtggctgggaatggg
gccggggcaccctggtcaccgtctcgagtggaggcggcggttcaggcggaggtggctctggcggtggcggaagtgcacagtctgccctgactc
agcctgcctccgtgtctgggtctcctggacagtcgatcaccgtctcctgcactggaaccagcagtgacgttggtgcttataactatgtctcctggtacc
aacaacacccaggcaaagcccccaaactcatgatttatgatgtcactactcggccctcaggggtttctaatcgcttctctggctccaagtctggcaac
acggcctccctgaccatctctgggctccaggctgaggacgaggctgattattactgcacctcatatactcgcagcagcactgtggtcttcggcggag
ggaccaagctgaccgtcctaggtgacgtacgcgagcccaaatcttctgacaaaactcacacatgcccaccgtgcccagcacctgaactcctgggt
ggaccgtcagtcttcctcttccccccaaaacccaaggacaccctcatgatctcccggacccctgaggtcacatgcgtggtggtggacgtgagccac
gaagaccctgaggtcaagttcaactggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgcaaggtctccaacaaagccctcccagccc
ccatcgagaaaaccatctccaaagccaaagggcagccccgagaaccacaggtgtacaccctgcccccatcccgggatgagctgaccaagaacc
aggtcagcctgacctgcctggtcaaaggcttctatccaagcgacatcgccgtggagtgggagagcaatgggcagccggagaacaactacaagac
cacgcctcccgtgctggactccgacggctccttcttcctctacagcaagctcaccgtggacaagagcaggtggcagcaggggaacgtcttctcatg
ctccgtgatgcatgaggctctgcacaaccactacacgcagaagagcctctccctgtctccgggtaaatga (SEQ ID NO: 653)
>Her162_Protein_leader-stop
MEAPAQLLFLLLLWLPDTTGEVQLVETGAEVKKPGASMKVSCKASGYSFTDYYMHWVRQA
PGQGLEWMGWINPNSGDTNYAQKFQGRVTMTRDTSITTAYMELSRLRSDDTAVYYCATER
YNSGWEWGRGTLVTVSSGGGGSGGGGSGGGGSAQSALTQPASVSGSPGQSITVSCTGTSSD
VGAYNYVSWYQQHPGKAPKLMIYDVTTRPSGVSNRFSGSKSGNTASLTISGLQAEDEADYY
CTSYTRSSTVVFGGGTKLTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 654)
>Her163_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcggtccatggctacggagactccgtggatgatgctc
ttgatgtctggggccgaggaaccctggtcaccgtctcgagtggaggcggcggttcaggcggaggtggctctggcggtggcggaagtgcacagtc
tgtgctgacgcagccgccctcagcgtctgggacccccgggcagacgatctccatctcttgttctggaagcaactccaacatcggaacttatagtgtta
gctggtaccagcagctcccacgagcggcccccagactcctcgtctatgctaatgatcgccggccctcaggggtccctgaccgattctctggctcca
agtctggcacctcagcctccctggccatcagtgggctccagtctgaggatgaggctgattattactgtgcagtatgggatgacaggttgaatggttttg
tcttcggaactgggaccaagctgaccgtcctaggtgacgtacgcgagcccaaatcttctgacaaaactcacacatgcccaccgtgcccagcacctg
aactcctgggtggaccgtcagtcttcctcttccccccaaaacccaaggacaccctcatgatctcccggacccctgaggtcacatgcgtggtggtgga
cgtgagccacgaagaccctgaggtcaagttcaactggtacgtggacggcgtggaggtgcataatgccaagacaaagccgcgggaggagcagta
caacagcacgtaccgtgtggtcagcgtcctcaccgtcctgcaccaggactggctgaatggcaaggagtacaagtgcaaggtctccaacaaagcc
ctcccagcccccatcgagaaaaccatctccaaagccaaagggcagccccgagaaccacaggtgtacaccctgcccccatcccgggatgagctg
accaagaaccaggtcagcctgacctgcctggtcaaaggcttctatccaagcgacatcgccgtggagtgggagagcaatgggcagccggagaac
aactacaagaccacgcctcccgtgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtaaatga (SEQ ID NO: 655)
>Her163_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAVHGYG
DSVDDALDVWGRGTLVTVSSGGGGSGGGGSGGGGSAQSVLTQPPSASGTPGQTISISCSGSN
SNIGTYSVSWYQQLPRAAPRLLVYANDRRPSGVPDRFSGSKSGTSASLAISGLQSEDEADYYC
AVWDDRLNGFVFGTGTKLTVLGDVREPKSSDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYT
QKSLSLSPGK. (SEQ ID NO: 656)
>Her164_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgaagtatggtggctacgacgctgatgcctttgatgt
ctggggccgagggacaatggtcaccgtctcgagtggaggcggcggttcaggcggaggtggctctggcggtggcggaagtgcacagtctgtcgt
gacgcagccgccctcagtgtctgcggccccaggacagaaggtcaccatctcctgctctggaagtagctccaacattggagattattatgtatcctgg
taccagcaactcccaggaacggcccccacactcctcatttatgacaatgataagcgaccctcagaagttcctgaccgattctctggctccaagtctgg
cacgtcggccaccctcggcatcaccggactccagactggggacgaggccgattattactgcacttcatgggatagcagcctgagtgctggggtgtt
cggcggagggaccaaggtcaccgtcctaggtgacgtacgcgagcccaaatcttctgacaaaactcacacatgcccaccgtgcccagcacctgaa
ctcctgggtggaccgtcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgcccccatcccgggatgagctgac
caagaaccaggtcagcctgacctgcctggtcaaaggcttctatccaagcgacatcgccgtggagtgggagagcaatgggcagccggagaacaa
ctacaagaccacgcctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga (SEQ ID NO: 657)
>Her164_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCAKYGGY
DADAFDVWGRGTMVTVSSGGGGSGGGGSGGGGSAQSVVTQPPSVSAAPGQKVTISCSGSSS
NIGDYYVSWYQQLPGTAPTLLIYDNDKRPSEVPDRFSGSKSGTSATLGITGLQTGDEADYYCT
SWDSSLSAGVFGGGTKVTVLGDVREPKSSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYT
QKSLSLSPGK. (SEQ ID NO: 658)
>Her165_CDS
atggaagcaccagcgcagcttctcttcctcctgctactctggctcccagataccaccggtaaggtgcagctggtgcagtctgggtctgagttgaaga
agcctggggcctcagtgaaggtttcctgccaggcttctggatacaccatcactaaccatagcatgaattgggtgcgacaggcccctgggcaaggg
cttgagtggatgggatggatcaacaccaacactgggaaccctacgtatgcccagggcttcacaggacggtttgtcttctccttggacacctctgcca
acacggcaactttgcagatcaccaacgtgcaggctgaggacacagccgtctactactgtgcgagagaggggagtatagacgtgtctggaacgcc
ctactactacggaatggacgcctgggggcaagggaccacggtcaccgtctcctcaggtggaggcggttcaggcggaggtggcagcggcggtg
gcggatcgcagtctgtgctgactcagcctgcctccgtgtctgggtctcctggacagtcgatcaccatctcctgcactggaaccagcagtgacgttgg
tggttataactatgtctcctggtaccaacaacacccaggcaaagcccccaaactcatgatttatgagggcagtgagcggccctcaggggttcctaat
cgcttctctggctccaagtctggcaacacggcctccctgacaatctctgggctccaggctgaggacgaggctgattattactgcagctcatatacaac
caggagcactcgagttttcggcggagggaccaagctgaccatcctaggtgacgtacgcgagcccaaatcttctgacaaaactcacacatgcccac
cgtgcccagcacctgaactcctgggtggaccgtcagtcttcctcttccccccaaaacccaaggacaccctcatgatctcccggacccctgaggtca
catgcgtggtggtggacgtgagccacgaagaccctgaggtcaagttcaactggtacgtggacggcgtggaggtgcataatgccaagacaaagcc
gcgggaggagcagtacaacagcacgtaccgtgtggtcagcgtcctcaccgtcctgcaccaggactggctgaatggcaaggagtacaagtgcaa
ggtctccaacaaagccctcccagcccccatcgagaaaaccatctccaaagccaaagggcagccccgagaaccacaggtgtacaccctgccccc
atcccgggatgagctgaccaagaaccaggtcagcctgacctgcctggtcaaaggcttctatccaagcgacatcgccgtggagtgggagagcaat
gggcagccggagaacaactacaagaccacgcctcccgtgctggactccgacggctccttcttcctctacagcaagctcaccgtggacaagagca
ggtggcagcaggggaacgtcttctcatgctccgtgatgcatgaggctctgcacaaccactacacgcagaagagcctctccctgtctccgggtaaat
ga (SEQ ID NO: 659)
>Her165_Protein_leader-stop
MEAPAQLLFLLLLWLPDTTGKVQLVQSGSELKKPGASVKVSCQASGYTITNHSMNWVRQAP
GQGLEWMGWINTNTGNPTYAQGFTGRFVFSLDTSANTATLQITNVQAEDTAVYYCAREGSI
DVSGTPYYYGMDAWGQGTTVTVSSGGGGSGGGGSGGGGSQSVLTQPASVSGSPGQSITISCT
GTSSDVGGYNYVSWYQQHPGKAPKLMIYEGSERPSGVPNRFSGSKSGNTASLTISGLQAEDE
ADYYCSSYTTRSTRVFGGGTKLTILGDVREPKSSDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHN
HYTQKSLSLSPGK. (SEQ ID NO: 660)
>Her166_CDS
atggaagcaccagcgcagcttctcttcctcctgctactctggctcccagataccaccggtcaggtgcagctggtgcagtctggggcagaaataaaa
aagccgggggagtctctgaagatctcctgtgagggttctggatacaggtttaccagccactggatcggctgggtgcgccagatgcccgggaaagg
cctggagtggatggggatcatctatcctggtgactctgataccagatacagcccgtccttccaaggccaggtcaccatctcagccgacaagtccatc
agcaccgcctacctgcagtggagcagcctgaaggcctcggacaccgccatgtattactgtgcgagacatagtgcgacgcatgatgcttttgatatct
ggggccggggcaccctggtcaccgtctcgagtggaggcggcggttcaggcggaggtggctctggcggtggcggaagtgcacagtctgtgttga
cgcagccgccctcagtgtctggggccccagggcagagggtcaccatctcctgtagtgggagcagctccaacatcgggacaggttacgatgttcac
tggtaccagcaacttccaggaacagcccccaaactcctcatctatagtttcaataagcggccctcaggggtccctgaccggttctctgcctccaagtc
tggcacctcagcctccctggtcatcactgggctccaggctgaggatgaggctgattattactgccagtcctatgacaatttgagtggtccccatgtggt
tttcggcacagggaccaagctgaccgtcctaggtgacgtacgcgagcccaaatcttctgacaaaactcacacatgcccaccgtgcccagcacctg
aactcctgggtggaccgtcagtcttcctcttccccccaaaacccaaggacaccctcatgatctcccggacccctgaggtcacatgcgtggtggtgga
cgtgagccacgaagaccctgaggtcaagttcaactggtacgtggacggcgtggaggtgcataatgccaagacaaagccgcgggaggagcagta
caacagcacgtaccgtgtggtcagcgtcctcaccgtcctgcaccaggactggctgaatggcaaggagtacaagtgcaaggtctccaacaaagcc
ctcccagcccccatcgagaaaaccatctccaaagccaaagggcagccccgagaaccacaggtgtacaccctgcccccatcccgggatgagctg
accaagaaccaggtcagcctgacctgcctggtcaaaggcttctatccaagcgacatcgccgtggagtgggagagcaatgggcagccggagaac
aactacaagaccacgcctcccgtgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtaaatga (SEQ ID NO: 661)
>Her166_Protein_leader-stop
MEAPAQLLFLLLLWLPDTTGQVQLVQSGAEIKKPGESLKISCEGSGYRFTSHWIGWVRQMPG
KGLEWMGIIYPGDSDTRYSPSFQGQVTISADKSISTAYLQWSSLKASDTAMYYCARHSATHD
AFDIWGRGTLVTVSSGGGGSGGGGSGGGGSAQSVLTQPPSVSGAPGQRVTISCSGSSSNIGTG
YDVHWYQQLPGTAPKLLIYSFNKRPSGVPDRFSASKSGTSASLVITGLQAEDEADYYCQSYD
NLSGPHVVFGTGTKLTVLGDVREPKSSDKTHTCPPCPAPELLGGPSVFLFPPKPKDTLMISRTP
EVTCVVVDVSHFDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKS
LSLSPGK. (SEQ ID NO: 662)
>Her167_CDS
atggaagcaccagcgcagcttctcttcctcctgctactctggctcccagataccaccggtcaggtgcagctgcaggagtcgggcccaggactggtg
aagccttcggagaccctgtccctcacctgcactgtctctggtggctccatcaccagtgatctttcctactggggctggctccgccagccccccggga
agggtctggagtggattgcgagtggtggtgacggtgagagcacctactacaacccgtccctcaacggtcgagtcaccttttccgtggacacgccca
agaaccaattctccctgaggctgagctctgtgaccgccgcagacacggctgtatattactgtgcgagacacccactctactattgtagtggtggtcgc
tgctactccgggaactttgacttttggggccagggaaccctggtcaccgtctcgagtggaggcggcggttcaggcggaggtggctctggcggtgg
cggaagtgcacaggctgtgctgactcagccgtcctcagcgtctgggacccccggtcagagggtcaccatttcttgttctggaacgacccccaatatt
ggaagtaattttgtctactggtatcaacaactcccagggacggcccccaaactcctcatctacaggaatgagcagcgcccttcaggggtccctgtcc
gattctctggctccaagtctggcacatcagcctccctggccatcagtgacctccggtccgaggatgaggctgactattactgtgcagcgtgggatga
cagcctgagtggtgtggtcttcggcggggggaccaagctgaccgtcctaggtgacgtacgcgagcccaaatcttctgacaaaactcacacatgcc
caccgtgcccagcacctgaactcctgggtggaccgtcagtcttcctcttccccccaaaacccaaggacaccctcatgatctcccggacccctgagg
tcacatgcgtggtggtggacgtgagccacgaagaccctgaggtcaagttcaactggtacgtggacggcgtggaggtgcataatgccaagacaaa
gccgcgggaggagcagtacaacagcacgtaccgtgtggtcagcgtcctcaccgtcctgcaccaggactggctgaatggcaaggagtacaagtg
caaggtctccaacaaagccctcccagcccccatcgagaaaaccatctccaaagccaaagggcagccccgagaaccacaggtgtacaccctgcc
cccatcccgggatgagctgaccaagaaccaggtcagcctgacctgcctggtcaaaggcttctatccaagcgacatcgccgtggagtgggagagc
aatgggcagccggagaacaactacaagaccacgcctcccgtgctggactccgacggctccttcttcctctacagcaagctcaccgtggacaagag
caggtggcagcaggggaacgtcttctcatgctccgtgatgcatgaggctctgcacaaccactacacgcagaagagcctctccctgtctccgggtaa
atga (SEQ ID NO: 663)
>Her167_Protein_leader-stop
MEAPAQLLFLLLLWLPDTTGQVQLQESGPGLVKPSETLSLTCTVSGGSITSDLSYWGWLRQP
PGKGLEWIASGGDGESTYYNPSLNGRVTFSVDTPKNQFSLRLSSVTAADTAVYYCARHPLYY
CSGGRCYSGNFDFWGQGTLVTVSSGGGGSGGGGSGGGGSAQAVLTQPSSASGTPGQRVTIS
CSGTTPNIGSNFVYWYQQLPGTAPKLLIYRNEQRPSGVPVRFSGSKSGTSASLAISDLRSEDEA
DYYCAAWDDSLSGVVFGGGTKLTVLGDVREPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK. (SEQ ID NO: 664)
>Her168_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaggggccggttcaccatctccagagacaattccaaga
acacgctgtatctgcaaatgaacagcctgagagccgaggacacggccgtgtattactgtgcaacttcgttgggttacggtgactttgactactgggg
gcgagggaccacggttaccgtctcgagtggaggcggcggttcaggcggaggtggctctggcggtggcggaagtgcacaggctgtgctgactca
gccgtcctcagcgtctggggcccccgggcacagggtcatcatctcttgttctggaagcagctccaacatcggaagttattatgtaagctggtaccag
cagctcccaggagcggcccccaaactcctcatctatcgtaatgatgagcggccctcaggggtccctgcccgattctctggctccacgtctggcacct
cagcctccctggccatcagtgggctccactctgaggatgaggctgattattattgtgcagcatgggatgacagcctgaatggtccggttttcggcgg
agggaccaaggtcaccgtcctaggtgacgtacgcgagcccaaatcttctgacaaaactcacacatgcccaccgtgcccagcacctgaactcctgg
gtggaccgtcagtcttcctcttccccccaaaacccaaggacaccctcatgatctcccggacccctgaggtcacatgcgtggtggtggacgtgagcc
acgaagaccctgaggtcaagttcaactggtacgtggacggcgtggaggtgcataatgccaagacaaagccgcgggaggagcagtacaacagca
cgtaccgtgtggtcagcgtcctcaccgtcctgcaccaggactggctgaatggcaaggagtacaagtgcaaggtctccaacaaagccctcccagcc
cccatcgagaaaaccatctccaaagccaaagggcagccccgagaaccacaggtgtacaccctgcccccatcccgggatgagctgaccaagaac
caggtcagcctgacctgcctggtcaaaggcttctatccaagcgacatcgccgtggagtgggagagcaatgggcagccggagaacaactacaaga
ccacgcctcccgtgctggactccgacggctccttcttcctctacagcaagctcaccgtggacaagagcaggtggcagcaggggaacgtcttctcat
gctccgtgatgcatgaggctctgcacaaccactacacgcagaagagcctctccctgtctccgggtaaatga (SEQ ID NO: 665)
>Her168_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVRGRFTISRDNSKNTLYLQMNSLRAEDTAVYYCATSLGY
GDFDYWGRGTTVTVSSGGGGSGGGGSGGGGSAQAVLTQPSSASGAPGHRVIISCSGSSSNIGS
YYVSWYQQLPGAAPKLLIYRNDERPSGVPARFSGSTSGTSASLAISGLHSEDEADYYCAAWD
DSLNGPVFGGGTKVTVLGDVREPKSSDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSL
SLSPGK. (SEQ ID NO: 666)
>Her169_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagaggctttggtgactactggggccgggggaca
atggtcaccgtctcgagtggaggcggcggttcaggcggaggtggctctggcggtggcggaagtgcactttcttctgagctgactcaggaccctgct
gtgtctgtggccttgggacagacagtcaggatcacatgccaaggagacagcctcagaagttattatgcaagctggtaccagcagaagccaggaca
ggcccctgtacttgtcatctatgctaaaaacaaccgaccctcagggatcccagaccgattctctggctccgactcaggaaacacagcttccttgacca
tcactggggctcaggcggaagatgaggctgactattactgtctctcccgggacagcagtggtaaccatctggtattcggcggagggaccaagctg
accgtcctaggtgacgtacgcgagcccaaatcttctgacaaaactcacacatgcccaccgtgcccagcacctgaactcctgggtggaccgtcagtc
ttcctcttccccccaaaacccaaggacaccctcatgatctcccggacccctgaggtcacatgcgtggtggtggacgtgagccacgaagaccctga
ggtcaagttcaactggtacgtggacggcgtggaggtgcataatgccaagacaaagccgcgggaggagcagtacaacagcacgtaccgtgtggt
cagcgtcctcaccgtcctgcaccaggactggctgaatggcaaggagtacaagtgcaaggtctccaacaaagccctcccagcccccatcgagaaa
accatctccaaagccaaagggcagccccgagaaccacaggtgtacaccctgcccccatcccgggatgagctgaccaagaaccaggtcagcctg
acctgcctggtcaaaggcttctatccaagcgacatcgccgtggagtgggagagcaatgggcagccggagaacaactacaagaccacgcctccc
gtgctggactccgacggctccttcttcctctacagcaagctcaccgtggacaagagcaggtggcagcaggggaacgtcttctcatgctccgtgatg
catgaggctctgcacaaccactacacgcagaagagcctctccccgtctccgggtaaatga (SEQ ID NO: 667)
>Her169_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARGFGD
YWGRGTMVTVSSGGGGSGGGGSGGGGSALSSELTQDPAVSVALGQTVRITCQGDSLRSYYA
SWYQQKPGQAPVLVIYAKNNRPSGIPDRFSGSDSGNTASLTITGAQAEDEADYYCLSRDSSG
NHLVFGGGTKLTVLGDVREPKSSDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSPSP
GK. (SEQ ID NO: 668)
>Her170_CDS
atggaagcaccagcgcagcttctcttcctcctgctactctggctcccagataccaccggtgaggtgcagctgttggagtctgggggaggcttggtac
agcctggggggtccctgagactctcctgtgcagcctctggattcacctttagcagctatgccatgagctgggtccgccaggctccagggaagggg
ctggagtgggtctcagctattagtggtagtggtggtagcacatactacgcagactccgtgaagggccggttcaccatctccagagacaattccaaga
acacgctgtatctgcaaatgaacagcctgagagccgaggacacggccgtgtattactgtgcgagaactacggcagatgcttttgatatctggggga
gggggaccacggtcaccgtctcgagtggaggcggcggttcaggcggaggtggctctggcggtggcggaagtgcactttcttctgagctgactca
ggaccctgctgtgtctgtggccctgggacagacagtcagcatcacatgccagggagacagcctcagaaacttttatgcaagctggtacctgcagaa
gccaggacaggccccaatacttgtcatctatggtaaaaacaagcggccctctgggatcccagaccgagtctctggctccagctcagaagacacag
cttccttgaccatcactggggctcaggcggaagatgaggctgactattactgtaactcccgggacagcagtggtaacgtggtcttcggcgggggga
ccaagctgaccgtcctaggtgacgtacgcgagcccaaatcttctgacaaaactcacacatgcccaccgtgcccagcacctgaactcctgggtgga
ccgtcagtcttcctcttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtacaacagcacgtac
cgtgtggtcagcgtcctcaccgtcctgcaccaggactggctgaatggcaaggagtacaagtgcaaggtctccaacaaagccctcccagcccccat
cgagaaaaccatctccaaagccaaagggcagccccgagaaccacaggtgtacaccctgcccccatcccgggatgagctgaccaagaaccaggt
cagcctgacctgcctggtcaaaggcttctatccaagcgacatcgccgtggagtgggagagcaatgggcagccggagaacaactacaagaccac
gcctcccgtgctggactccgacggctccttcttcctctacagcaagctcaccgtggacaagagcaggtggcagcaggggaacgtcttctcatgctc
cgtgatgcatgaggctctgcacaaccactacacgcagaagagcctctccccgtctccgggtaaatga (SEQ ID NO: 669)
>Her170_Protein_leader-stop
MEAPAQLLFLLLLWLPDTTGEVQLLESGGGLVQPGGSLRLSCAASGFTFSSYAMSWVRQAP
GKGLEWVSAISGSGGSTYYADSVKGRFTISRDNSKNTLYLQMNSLRAEDTAVYYCARTTAD
AFDIWGRGTTVTVSSGGGGSGGGGSGGGGSALSSELTQDPAVSVALGQTVSITCQGDSLRNF
YASWYLQKPGQAPILVIYGKNKRPSGIPDRVSGSSSEDTASLTITGAQAEDEADYYCNSRDSS
GNVVFGGGTKLTVLGDVREPKSSDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSPS
PGK. (SEQ ID NO: 670)
> Stumpy Peptide: Her2 N-terminal membrane proximal region of p95 (amino acid 645-656 of
HER2) fused with EKK sequence
EQRASPLTSIIS-EKK (SEQ ID NO: 671)
> Scrambled Peptide: Scrambled sequence, used as a negative control/de-selection protein
PEISLSQRITAS-EKK (SEQ ID NO: 672)

1. A binding protein that specifically binds ErbB2, wherein the binding protein is an ErbB2 agonist.

2. The binding protein of claim 1 which reduces cellular proliferation in an ErbB2-expressing cancer cell.

3. The binding protein of claim 1 which increases apoptosis in an ErbB2-expressing tumor.

4. The binding protein of claim 1 which reduces the growth of an ErbB2-expressing tumor.

5. The binding protein of claim 2 wherein the ErbB2-expressing cancer cell is a breast cancer cell.

6. The binding protein of claim 2 wherein the ErbB2 expressing cancer cell is from a cell line selected from the group consisting of: SKBR3, BT474, MDA-MB-453 and MDA-MB-361.

7. A binding protein that specifically binds ErbB2, wherein the binding protein preferentially binds an ErbB2 extracellular domain (ECD) homo-dimer over ErbB2 ECD monomer and shed ErbB2 ECD.

8. The binding protein of claim 1, wherein the binding protein preferentially binds an ErbB2 extracellular domain (ECD) homo-dimer over ErbB2 ECD monomer and shed ErbB2 ECD.

9. The binding protein of claim 2, that possesses one or more or the following properties:
(a) increases ErbB2 phosphorylation in a breast cancer cell;
(b) increases the phosphorylation of one or more of AKT, MAPK, MEK, ERK1 and ERK2;
(c) binds ErbB2 ECD in a location selected from the group consisting of: the L1/CR1 domain, the L2 domain, the CR2 domain;
(d) internalizes in an ErbB2-expressing cell;
(e) decreases ErbB2 ECD shedding compared to cells not treated with the binding protein;
(f) decreases the amount of cell surface ErbB2 compared to cells not treated with the binding protein;
(g) binds macaque ErbB2;
(h) binds mouse ErbB2;
(i) has Fc dependent cellular cytotoxicity activity with ErbB2-expressing cells;
(i) is stable in plasma for at least 96 hours;
(k) has antiproliferative activity that is reversible by inhibiting MEK-mediated phosphorylation, ERK2-mediated phosphorylation, or both; or
(l) enhances the cytotoxic effect of a chemotherapeutic.

10. The binding protein of claim 1 which is an antibody, an antigen-binding fragment of an antibody or a small modular immunopharmaceutical (SMIP).

11. The binding protein of claim 10 which is an antigen-binding fragment of an antibody, wherein the antigen-binding fragment is selected from the group consisting of: a Fab fragment, an F(ab′)2 fragment, an scFv, a dAb, and Fv fragment and a VHH.

12. The binding protein of claim 1, which is human antibody or an antigen-binding fragment thereof.

13. The binding protein of claim 1, wherein the ErbB2 is human ErbB2 (SEQ ID NO: 246).

14. A binding protein that specifically binds ErbB2, wherein the binding protein comprises:
(a) a VH domain or an antigen-binding portion thereof comprising the CDR1, CDR2 and CDR3 amino acid sequences set forth in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65, 67, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, 415, 419, 423, 427, 431, 435, 439, 443, 447, 451, 455, 459, 463, 467, 471, 475, 479, 483, 487, 491, 495, 499, 503, 507, 511, 515, 519 and 523; or
(b) a VL domain or an antigen-binding portion thereof comprising the CDR1, CDR2 and CDR3 amino acid sequences set forth in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94, 95, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 401, 405, 409, 413, 417, 421, 425, 429, 433, 437, 441, 445, 449, 453, 457, 461, 465, 469, 473, 477, 481, 485, 489, 493, 497, 501, 505, 509, 513, 517, 521 and 525; or
(c) a VH domain or an antigen-binding portion thereof of (a) and a VL domain or an antigen-binding portion thereof of (b).

15. The binding protein of claim 14, comprising the VH CDR1, CDR2 and CDR3 amino acid sequences and the VL CDR1, CDR2 and CDR3 sequences of any one of: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H11, S6R3_CS_{—}1G5, S6R2_DP47_{—}1H1, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9.

16. A binding protein that specifically binds ErbB2, wherein the binding protein comprises:
(a) a VH domain having an amino acid sequence that is at least 90% identical to any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65, 67, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, 415, 419, 423, 427, 431, 435, 439, 443, 447, 451, 455, 459, 463, 467, 471, 475, 479, 483, 487, 491, 495, 499, 503, 507, 511, 515, 519 and 523; or
(b) a VL domain having an amino acid sequence that is at least 90% identical to any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94, 95, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 401, 405, 409, 413, 417, 421, 425, 429, 433, 437, 441, 445, 449, 453, 457, 461, 465, 469, 473, 477, 481, 485, 489, 493, 497, 501, 505, 509, 513, 517, 521 and 525; or
(c) a VH domain of (a) and a VL domain of (b); or
(d) VH domain and VL domain amino acid sequence that are at least 90% identical to the VH and VL, amino acid sequences, respectively, in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12D1, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H11, S6R3_CS_{—}1G5, S6R2_DP47_{—}1H11, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9.

17. The binding protein of claim 16, wherein the binding protein comprises:
(a) a VH domain having an amino acid sequence that is at least 95% identical to any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65, 67, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, 415, 419, 423, 427, 431, 435, 439, 443, 447, 451, 455, 459, 463, 467, 471, 475, 479, 483, 487, 491, 495, 499, 503, 507, 511, 515, 519 and 523; or
(b) a VL domain having an amino acid sequence that is at least 95% identical to any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94, 95, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 401, 405, 409, 413, 417, 421, 425, 429, 433, 437, 441, 445, 449, 453, 457, 461, 465, 469, 473, 477, 481, 485, 489, 493, 497, 501, 505, 509, 513, 517, 521 and 525; or
(c) a VH domain of (a) and a VL domain of (b); or
(d) VH and VL amino acid sequences that are at least 95% identical to the VH and VL amino acid sequences, respectively, in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}10B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}1C1, S1R3A1_DP47_{—}4G1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H11, S6R3_CS_{—}1G5, S6R2_DP47_{—}1H11, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9.

18. The binding protein of claim 16, wherein the binding protein comprises:
(a) a VH domain having the amino acid sequence of any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65 or 67, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, 415, 419, 423, 427, 431, 435, 439, 443, 447, 451, 455, 459, 463, 467, 471, 475, 479, 483, 487, 491, 495, 499, 503, 507, 511, 515, 519 and 523; or
(b) a VL domain having the amino acid sequence of any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94, 95, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 401, 405, 409, 413, 417, 421, 425, 429, 433, 437, 441, 445, 449, 453, 457, 461, 465, 469, 473, 477, 481, 485, 489, 493, 497, 501, 505, 509, 513, 517, 521 and 525; or
(c) a VH domain of (a) and a VL domain of (b); or
(d) VH and VL amino acid sequences of the VH and VL amino acid sequences, respectively, in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_IC₁₂, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, S1R3B1_BMV_{—}1A1, S1R3B1_DP47_{—}3A2, S1R3A1_DP47_{—}11B7, S1R3A1_DP47_{—}11D1, S1R3A1_DP47_{—}7F3, S1R2B_DP47_{—}4E3, S1R3C1_DP47_{—}2G2, S1R3A1_DP47_{—}11H6, S1R3A1_BMV_{—}3B1, S1R3A1_DP47_{—}6B9, S1R2A_CS_{—}110B8, S1R3A1_DP47_{—}7A6, S1R3B2_DP47_{—}2G3, S1R2B_CS_{—}6H11, S1R3A1_DP47_{—}10G1, S1R3A1_DP47_{—}7C1, S1R2A_DP47_{—}5D6, S1R3A1_DP47_{—}11F6, S1R3A1_DP47_{—}11D3, S1R3A1_CS_{—}8A8, S1R3A1_BMV_{—}5D10, S1R3A1_DP47_{—}11C1, S1R3A1_DP47_{—}4E1, S1R3A1_DP47_{—}10E1, S1R3A1_CS_{—}11C3, S1R3A1_CS_{—}13H11, S1R3A1_CS_{—}2D9, S1R2A_CS_{—}3D4, S1R3A1_DP47_{—}2H6, S1R3A1_DP47_{—}4G1, S1R2A_DP47_{—}3C1, S1R3A1_DP47_{—}7B2, S1R3B2_DP47_{—}4E2, S1R3A1_CS_{—}16C2, S1R3A1_CS_{—}11E5, S1R3A1_CS_{—}16D7, S1R2A_CS_{—}10B10, S1R3A1_CS_{—}15C2, S1R3A1_CS_{—}9C1, S1R2A_CS_{—}5A1, S1R2A_CS_{—}8C8, S1R3A1_CS_{—}13H5, S1R2B_CS_{—}5E9, S1R3A1_CS_{—}8F9, S1R3A1_CS_{—}14B5, S1R2A_CS_{—}9E10, S1R3A1_CS_{—}7A10, S1R3A1_BMV_{—}6H7, S1R3A1_CS_{—}12A11, S1R3A1_CS_{—}13D12, S1R3A1_CS_{—}7A8, S1R2A_CS_{—}2C9, S1R3A1_CS_{—}12D1, S1R2A_CS_{—}7D4, S1R3A1_CS_{—}15B8, S6R3_DP47_{—}1A10, S6R2_DP47_{—}1E11, S5R2_DP47_{—}1H11, S6R3_CS_{—}1G5, S6R2_DP47_{—}1H11, S5R3_DP47_{—}1A10, S5R2_DP47_{—}1D11, S5R2_CS_{—}1A8, S6R3_CS_{—}1B7, S6R2_CS_{—}1E5, S6R3_BMV_{—}1C2, S5R2_DP47_{—}1B10, S6R3_DP47_{—}1C12, S5R2_DP47_{—}1D10, and S6R3_DP47_{—}1H9.

19. The binding protein of claim 14 or 16, which is a SMIP.

20. A SMIP comprising an amino acid sequence that is at least 90% identical to the amino acid sequence of any one of SEQ ID NOS: 159, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668 and 670, excluding the leader sequence.

21. The SMIP of claim 20, comprising an amino acid sequence that is at least 95% identical to the amino acid sequence of any one of SEQ ID NOS: 159, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668 and 670, excluding the leader sequence.

22. The SMIP of claim 20, comprising the amino acid sequence of any one of SEQ ID NOS: 159, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668 and 670, excluding the leader sequence.

23. The SMIP according to claim 20, that specifically binds a polypeptide selected from the group consisting of:
(a) a polypeptide comprising the amino acid sequence as set forth in SEQ ID NO. 242;
(b) a polypeptide comprising the amino acid sequence as set forth in SEQ ID NO. 243;
(c) a polypeptide comprising the amino acid sequence as set forth in SEQ ID NO. 244;
(d) a polypeptide comprising the amino acid sequence as set forth in SEQ ID NO. 245; and
(e) a polypeptide comprising the amino acid sequence as set forth in SEQ ID NO. 671.

24. The SMIP according to claim 20, that specifically binds ERBB2 in a region selected from the group consisting of:
(a) a region of the extracellular domain comprising the amino acid sequence ASPLTS;
(b) a region of the extracellular domain comprising the amino acid sequence EQRASPLTS; and
(c) a region of the extracellular domain comprising the amino acid sequence EQRASPLTSIIS.

25. A nucleic acid molecule encoding the SMIP of claim 20.

26. A nucleic acid molecule that encodes a binding protein that specifically binds ErbB2, wherein the nucleic acid molecule comprises a nucleotide sequence selected from:
(a) the nucleotide sequence of any one of SEQ ID NOS: 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156; 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520 or 524; or
(b) the nucleotide sequence of any one of SEQ ID NOS: 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157; 254, 258, 262, 266, 270, 274, 278, 282, 286, 290, 294, 298, 302, 306, 310, 314, 318, 322, 326, 330, 334, 338, 342, 346, 350, 354, 358, 362, 366, 370, 374, 378, 382, 386, 390, 394, 398, 402, 406, 410, 414, 418, 422, 426, 430, 434, 438, 442, 446, 450, 454, 458, 462, 466, 470, 474, 478, 482, 486, 490, 494, 498, 502, 506, 510, 514, 518, 522, or 526; or
(c) both the nucleotide sequence of (a) and the nucleotide sequence of (b).

27. The nucleic acid molecule of claim 26, comprising the nucleotide sequence of any one of SEQ ID NOS: 158, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230,r 232, 533, 535, 537, 539, 541, 543, 545, 547, 549, 551, 553, 555, 557, 559, 561, 563, 565, 657, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667 and 669.

28. A composition comprising SMIP of claim 20.

29. The composition of claim 28, further comprising an additional therapeutic or diagnostic agent.

30. A composition comprising two or more SMIPS of claim 20.

31. The composition of claim 30, wherein the two or more SMIPS do not cross-block each other for binding to ErbB2.

32. The composition of claim 29 that comprises an additional therapeutic agent, wherein the therapeutic agent is a chemotherapeutic or anti-inflammatory agent.

33. A host cell comprising a nucleic acid molecule of claim 26.

34. The host cell of claim 33 selected from the group consisting of an HEK cell, an NSO cell and a CHO cell.

35. A method for producing a binding molecule that specifically binds ErbB2, or a SMIP that specifically binds ErbB2, comprising the step of culturing the host cell of claim 33 under conditions the permit protein expression.

36. A method for reducing ErbB2-mediated proliferation of a cancer cell comprising the step of administering to a subject or mammal in need thereof an effective amount of the composition of claim 28.

37. A method for reducing tumor growth of an ErbB2-expressing tumor, comprising administering to a subject or mammal in need thereof an effective amount of the composition of claim 28.

38. A method for increasing apoptosis in an ErbB2-expressing tumor, comprising administering to a subject or mammal in need thereof an effective amount of the composition of claim 28.

39. The method of claim 36 or 37, further comprising administering a chemotherapeutic agent.

40. The method of claim 39, wherein the chemotherapeutic agent is selected from the group consisting of: Taxol, Doxorubicin, Gemcitabine and Cisplatin.

41. A method for reducing ErbB2 ectodomain shedding in an ErbB2-expressing cell comprising the step of contacting the cell with a SMIP of claim 20.

42. A method for reducing the amount of cell surface ErbB2 in a cell comprising the step of contacting the cell with a SMIP of claim 20.

43. The binding protein of claim 1, which is detectably labeled.

44. A method for detecting an ErbB2 expressing tumor in a subject, comprising administering the binding protein of claim 43.

45. A method for detecting ErbB2 in a sample from a subject comprising the step of contacting the sample with a binding protein of claim 14 or 16, or a SMIP of claim 20 under conditions that permit binding and detecting binding, wherein binding indicates the presence of ErbB2.

46. A method of treating cancer characterized by ErbB2 expression comprising administering to a mammal or subject in need thereof an effective amount of a SMIP of claim 20.
